(19) United States
(12) Patent Application Publication
(10) Pub. No.: US 2023/0172059 A1
(43) Pub. Date: Jun. 1, 2023

(54) LIGHT EMITTING ELEMENT AND AMINE COMPOUND FOR THE SAME

(71) Applicant: Samsung Display Co., Ltd., Yongin-si (KR)

(72) Inventor: Takuya UNO, Yokohama (JP)

(21) Appl. No.: 17/963,871

(22) Filed: Oct. 11, 2022

(30) Foreign Application Priority Data
Dec. 1, 2021 (KR) 10-2021-0169949

(51) Int. Cl. H01L 51/00; C07D 307/91; C07D 333/76

(52) U.S. Cl. CPC H01L 51/0061; H01L 51/006; C07D 307/91; C07D 333/76; H01L 51/5056

(57) ABSTRACT

A light emitting element and an amine compound for the light emitting element are provided. The light emitting element of an embodiment includes a first electrode, a second electrode disposed on the first electrode and at least one functional layer disposed between the first electrode and the second electrode, wherein the functional layer includes the amine compound represented by a specific chemical structure, thereby improving the emission efficiency and element life of the light emitting element.

### CROSS-REFERENCE TO RELATED APPLICATIONS

This application claims priority to and the benefit of Korean Patent Application No. 10-2021-0169949, filed on Dec. 1, 2021, the entire content of which is hereby incorporated by reference.

### BACKGROUND

### 1. Field

The present disclosure herein relates to an amine compound and a light emitting element including the same, and particularly, to a light emitting element including a novel amine compound in a hole transport region.

### 2. Description of the Related Art

Recently, the development of an organic electroluminescence display device as an image display device is being actively conducted. The organic electroluminescence display device is a so-called self-luminescent type or kind light emitting element in which holes and electrons injected from a first electrode and a second electrode recombine in an emission layer such that a light emitting material in the emission layer emits light to achieve display (e.g., to display an image).

In the application of a light emitting element to a display device, there is a desire for a light emitting element having low driving voltage, high luminous efficiency, and/or a long service life (e.g., long lifespan), and the development on materials for a light emitting element capable of stably attaining such characteristics is being continuously pursued (e.g., required).

In some embodiments, in order to accomplish a light emitting element with high efficiency, development on materials for a hole transport region capable of suppressing the diffusion of the exciton energy of an emission layer is being pursued.

### SUMMARY

Aspects according to embodiments of the present disclosure are directed toward a light emitting element showing excellent or suitable emission efficiency and long-life characteristics.

Aspects according to embodiments of the present disclosure are directed toward an amine compound which is a material for a light emitting element having high efficiency and long-life characteristics.

According to an embodiment of the present disclosure, an amine compound is represented by Formula 1.

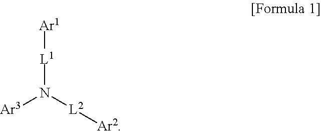

In Formula 1, L¹ and L² may each independently be a direct linkage, a substituted or unsubstituted arylene group having 6 to 30 ring-forming carbon atoms, or a substituted or unsubstituted heteroarylene group having 5 to 30 ring-forming carbon atoms, Ar¹ and Ar² may each independently be a substituted or unsubstituted aryl group having 6 to 30 ring-forming carbon atoms, or a substituted or unsubstituted heteroaryl group having 5 to 30 ring-forming carbon atoms, when Ar¹ or Ar² is a substituted or unsubstituted fluorenyl group, any one selected from among two benzene rings forming the 3-ring fluorenyl group is bonded with L¹ or L², or bonded with the nitrogen atom of Formula 1, L¹-Ar¹ and L²-Ar² do not include a carbazole group or a silyl group (e.g., do not include any carbazole group and do not include any silyl group), and Ar³ is different from Ar¹ and Ar², and is represented by Formula 2.

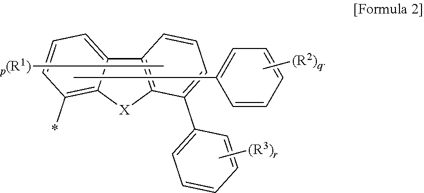

In Formula 2, X is O or S, R¹ is a hydrogen atom, a deuterium atom, a halogen atom, or a substituted or unsubstituted alkyl group having 1 to 20 carbon atoms, R² and R³ may each independently be a hydrogen atom, a deuterium atom, a halogen atom, a substituted or unsubstituted alkyl group having 1 to 20 carbon atoms, or a substituted or unsubstituted aryl group having 6 to 30 ring-forming carbon atoms, or combined with an adjacent group to form a hydrocarbon ring, “p”, “q” and “r” may each independently be an integer of 0 to 5, and *- is a position combined with the nitrogen atom of Formula 1.

In an embodiment, Formula 2 may be represented by any one selected from among Formula 2-1 to Formula 2-4.

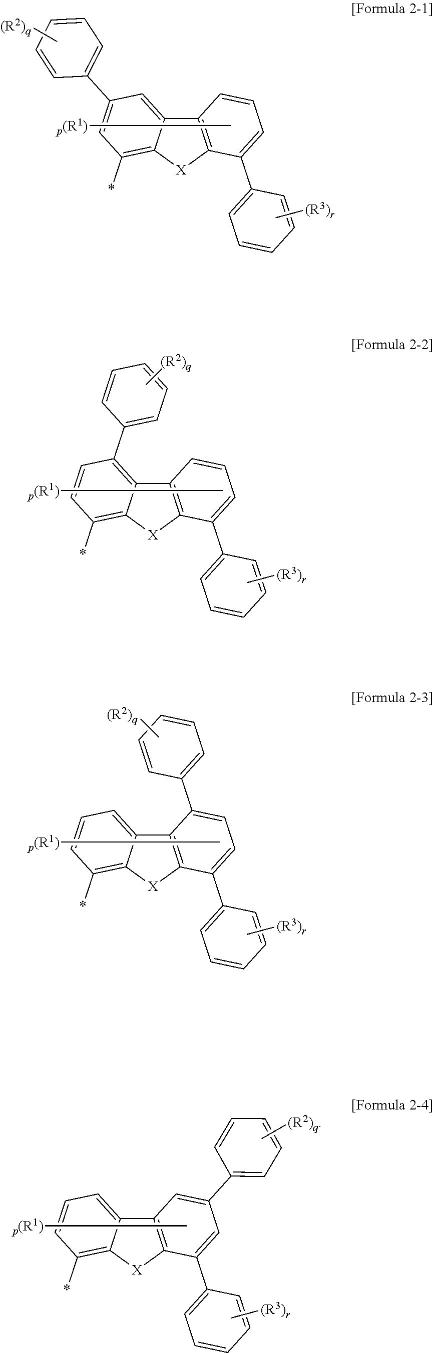

In Formula 2-1 to Formula 2-4, X, R¹, R², R³, “p”, “q” and “r” may each independently be the same as defined in Formula 2.

In an embodiment, the amine compound may be represented by Formula 3.

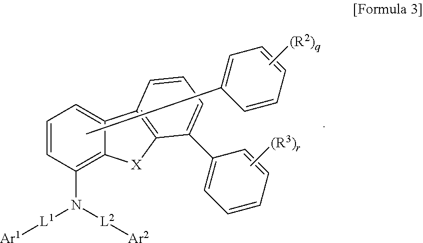

In Formula 3, X, L¹, L², Ar¹, Ar², R², R³, “q” and “r” may each independently be the same as defined in Formula 1 and Formula 2.

In an embodiment, Formula 3 may be represented by any one selected from among Formula 3-1 to Formula 3-4.

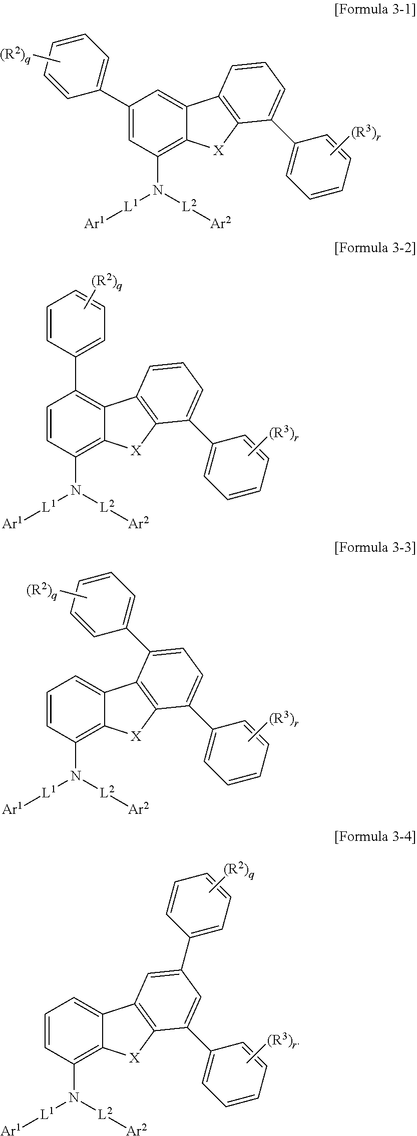

In Formula 3-1 to Formula 3-4, X, L¹, L², Ar¹, Ar², R², R³, “q” and “r” may each independently be the same as defined in Formula 1 and Formula 2.

In an embodiment, the amine compound may be represented by Formula 4.

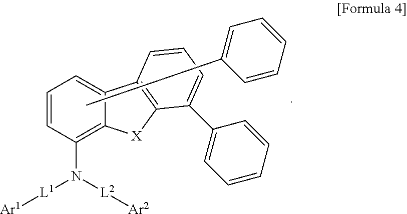

In Formula 4, X, L¹, L², Ar¹, and Ar² may each independently be the same as defined in Formula 1.

According to an embodiment of the present disclosure, a light emitting element includes: a first electrode; a second electrode on the first electrode; and at least one functional layer between the first electrode and the second electrode, and including the amine compound of an embodiment.

In an embodiment, the at least one functional layer may include an emission layer, a hole transport region between the first electrode and the emission layer, and an electron transport region between the emission layer and the second electrode, and the hole transport region may include the amine compound.

In an embodiment, the hole transport region may include a hole injection layer, and a hole transport layer on the hole injection layer, and the hole transport layer may include the amine compound.

In an embodiment, the hole transport region may include a hole injection layer, a hole transport layer, and an electron blocking layer, stacked in stated order, and the electron blocking layer may include the amine compound.

### BRIEF DESCRIPTION OF THE DRAWINGS

The accompanying drawings are included to provide a further understanding of the present disclosure and are incorporated in and constitute a part of this specification. The drawings illustrate embodiments of the present disclosure and, together with the description, serve to explain principles of the present disclosure. In the drawings:

FIG. 1 is a plan view showing a display apparatus according to an embodiment;

FIG. 2 is a cross-sectional view showing a part corresponding to the line I-I′ in FIG. 1;

FIG. 3 is a cross-sectional view schematically showing a light emitting element of an embodiment;

FIG. 4 is a cross-sectional view schematically showing a light emitting element of an embodiment;

FIG. 5 is a cross-sectional view schematically showing a light emitting element of an embodiment;

FIG. 6 is a cross-sectional view schematically showing a light emitting element of an embodiment;

FIG. 7 is a cross-sectional view showing a display apparatus according to an embodiment;

FIG. 8 is a cross-sectional view showing a display apparatus according to an embodiment;

FIG. 9 is a cross-sectional view showing a display apparatus according to an embodiment; and

FIG. 10 is a cross-sectional view showing a display apparatus according to an embodiment.

### DETAILED DESCRIPTION

The present disclosure may have one or more suitable modifications and may be embodied in different forms, and example embodiments will be explained in more detail with reference to the accompany drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure.

In the description, it will be understood that when an element (or region, layer, part, etc.) is referred to as being “on”, “connected to” or “coupled to” another element, it can be directly on, connected or coupled to the other element or a third intervening element may be present.

Like reference numerals refer to like elements throughout, and duplicative descriptions thereof may not be provided. In some embodiments, in the drawings, the thickness, the ratio, and the dimensions of constituent elements may be exaggerated for effective explanation of technical contents. The term “and/or” includes one or more combinations which may be defined by relevant elements.

It will be understood that, although the terms first, second, etc. may be used herein to describe various elements, these elements should not be limited by these terms. These terms are only used to distinguish one element from another element. Thus, a first element could be termed a second element without departing from the teachings of the present disclosure. Similarly, a second element could be termed a first element. As used herein, the singular forms are intended to include the plural forms as well, unless the context clearly indicates otherwise.

In some embodiments, the terms “below”, “beneath”, “on” and “above” are used for explaining the relation of elements shown in the drawings. The terms are relative concept and are explained based on the direction shown in the drawing.

In the description, it will be further understood that the terms “comprises” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

Unless otherwise defined, all terms (including technical and scientific terms) used herein have the same meaning as commonly understood by one of ordinary skill in the art to which this present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the description, when a layer, a film, a region, a plate, etc. is referred to as being “on” or “above” another part, it can be “directly on” the other part, or intervening layer(s) may also be present. In contrast, when a layer, a film, a region, a plate, etc. is referred to as being “under” or “below” another part, it can be “directly under” the other part, or intervening layer(s) may also be present. Also, when an element is referred to as being disposed “on” another element, it can be disposed under the other element.

In the description, the term “substituted or unsubstituted” corresponds to an unsubstituted group or a group substituted with at least one substituent selected from the group consisting of a deuterium atom, a halogen atom, a cyano group, a nitro group, an amine group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the example substituents may be substituted or unsubstituted. For example, a biphenyl group may be interpreted as an aryl group or a phenyl group substituted with a phenyl group.

In the description, the term “forming a ring via the combination with an adjacent group” may refer to forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In addition, the ring formed via the combination with an adjacent group may be combined with another ring to form a spiro structure.

In the description, the term “adjacent group” may refer to a substituent substituted for an atom which is directly linked to (e.g., combined with) an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

In the description, a halogen atom may be a fluorine atom, a chlorine atom, a bromine atom, or an iodine atom.

In the description, an alkyl group may be a linear, branched, or cyclic alkyl group. The number of carbon atoms in the alkyl group may be 1 to 60, 1 to 50, 1 to 30, 1 to 20, 1 to 10, or 1 to 6. Non-limiting examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc.

In the description, the term “hydrocarbon ring group” refers to an optional functional group or substituent derived from an aliphatic hydrocarbon ring or a fused ring of an aliphatic hydrocarbon ring group and an aromatic hydrocarbon ring group. The number of the ring-forming carbon atoms in the hydrocarbon ring group may be 5 to 60, 5 to 30, or 6 to 30.

In the description, an aryl group refers to an optional functional group or substituent derived from an aromatic hydrocarbon ring. The aryl group may be a monocyclic aryl group or a polycyclic aryl group. The number of carbon atoms for forming rings in the aryl group may be 6 to 30, 6 to 20, or 6 to 15. Non-limiting examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc.

In the description, a heterocyclic group refers to an optional functional group or substituent derived from a ring including one or more selected from among B, O, N, P, Se, Si, and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

When the heterocyclic group includes two or more heteroatoms, the two or more heteroatoms may be the same or different. The heterocyclic group may be a monocyclic heterocyclic group or a polycyclic heterocyclic group, and has the concept including a heteroaryl group. The number of carbon atoms for forming rings of the heterocyclic group may be 2 to 60, 2 to 30, 2 to 20, and 2 to 10.

In the description, an aliphatic heterocyclic group may include one or more selected from among B, O, N, P, Se, Si, and S as heteroatoms. The number of ring-forming carbon atoms of the aliphatic heterocyclic group may be 2 to 30, 2 to 20, or 2 to 10. Non-limiting examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc.

In the description, a heteroaryl group may include one or more selected from among B, O, N, P, Se, Si, and S as heteroatoms. When the heteroaryl group includes two or more heteroatoms, two or more heteroatoms may be the same or different. The heteroaryl group may be a monocyclic heterocyclic group or polycyclic heterocyclic group. The number of carbon atoms for forming rings of the heteroaryl group may be 2 to 30, 2 to 20, or 2 to 10. Non-limiting examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, benzoxazole, benzimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc.

In the description, the same explanation on the above-described aryl group may be applied to an arylene group except that the arylene group is a divalent group. The same explanation on the above-described heteroaryl group may be applied to a heteroarylene group except that the heteroarylene group is a divalent group.

In the description, a fluorenyl group may be substituted, and two substituents may be combined with each other to form a spiro structure. Examples of a substituted fluorenyl group are as follows, but the present disclosure is not limited thereto.

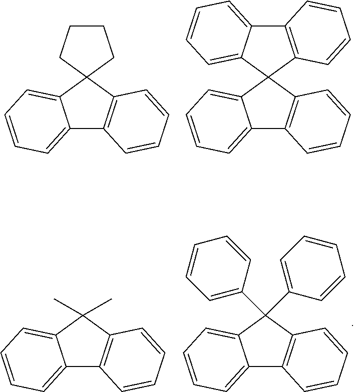

In the description, a silyl group includes an alkyl silyl group and an aryl silyl group. Non-limiting examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc.

In the description, a thio group may include an alkyl thio group and an aryl thio group. The thio group may refer to the above-defined alkyl group or aryl group combined with a sulfur atom. Non-limiting examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc.

In the description, an oxy group may refer to the above-defined alkyl group or aryl group which is combined with an oxygen atom. The oxy group may include an alkoxy group and an aryl oxy group. The alkoxy group may be a linear, branched or cyclic chain. The number of carbon atoms of the alkoxy group is not specifically limited but may be, for example, 1 to 60, 1 to 20 or 1 to 10. The number of ring-forming carbon atoms of the aryl oxy group is not specifically limited, but may be, for example, 6 to 60, 6 to 30, or 6 to 20. Non-limiting examples of the oxy group may include methoxy, ethoxy, n-propoxy, isopropoxy, butoxy, pentyloxy, hexyloxy, octyloxy, nonyloxy, decyloxy, benzyloxy, etc. However, the present disclosure is not limited thereto.

In the description, a boron group may refer to the above-defined alkyl group or aryl group, combined with a boron atom. The boron group includes an alkyl boron group and an aryl boron group. Non-limiting examples of the boron group include a dimethylboron group, a diethylboron group, a t-butylmethylboron group, a diphenylboron group, a phenylboron group, etc.

In the description, the number of carbon atoms in an amine group is not specifically limited, but may be 1 to 30. The amine group may include an alkyl amine group and an aryl amine group. Non-limiting examples of the amine group include a methylamine group, a dimethylamine group, a phenylamine group, a diphenylamine group, a naphthylamine group, a 9-methyl-anthracenylamine group, a triphenylamine group, etc.

In the description, alkyl groups in an alkylthio group, alkylsulfoxy group, alkylaryl group, alkylamino group, alkylboron group, alkyl silyl group, and alkyl amine group may be the same as the examples of the above-described alkyl group.

In the description, aryl groups in an aryloxy group, arylthio group, arylsulfoxy group, aryl amino group, arylboron group, and aryl silyl group may be the same as the examples of the above-described aryl group.

In the description, a direct linkage may refer to a single bond. In some embodiments, in the description, “-*” refers to a position to be connected.

Hereinafter, the light emitting element of an embodiment will be explained referring to the drawings.

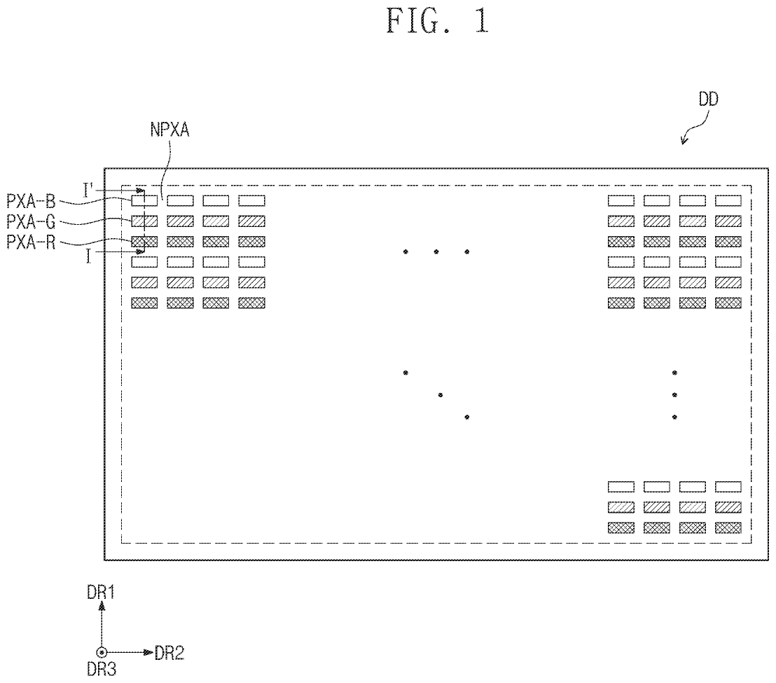

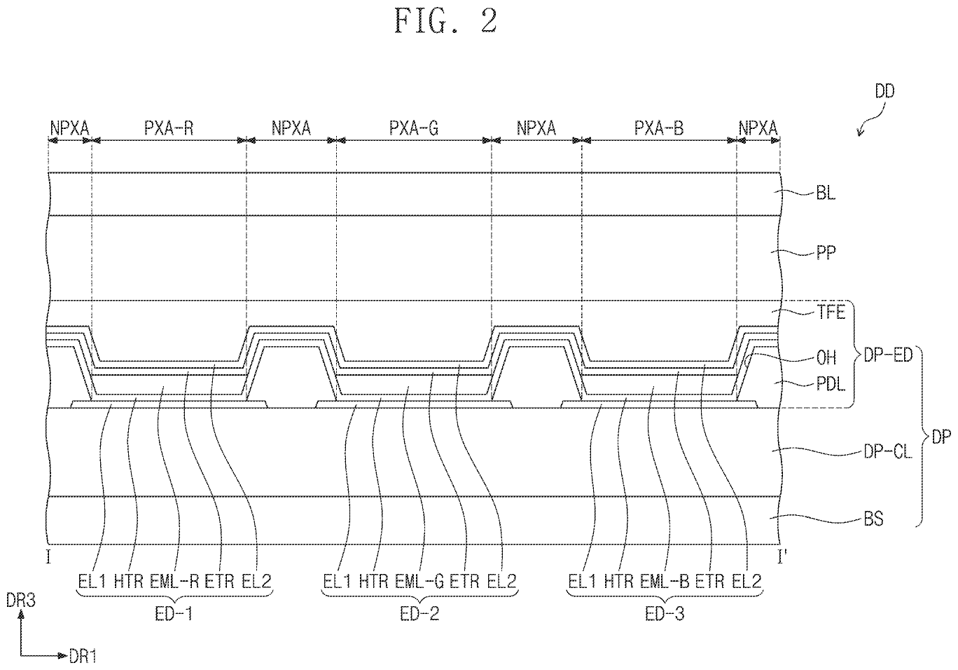

FIG. 1 is a plan view showing an embodiment of a display apparatus DD. FIG. 2 is a cross-sectional view of a display apparatus DD of an embodiment. FIG. 2 is a cross-sectional view showing a part corresponding to the line I-I′ of FIG. 1.

The display apparatus DD may include a display panel DP and an optical layer PP disposed on the display panel DP. The display panel DP may include light emitting elements ED-1, ED-2 and ED-3. The display apparatus DD may include multiple light emitting elements ED-1, ED-2 and ED-3. The optical layer PP may be disposed on the display panel DP and control reflection of external light by the display panel DP. The optical layer PP may include, for example, a polarization layer or a color filter layer. In some embodiments, different from the drawings, the optical layer PP may not be provided in the display apparatus DD.

On the optical layer PP, a base substrate BL may be disposed. The base substrate BL may be a member providing a base surface where the optical layer PP is disposed. The base substrate BL may be a glass substrate, a metal substrate, a plastic substrate, etc. However, the present disclosure is not limited thereto, and the base substrate BL may be an inorganic layer, an organic layer, or a composite material layer. In some embodiments, different from the drawings, the base substrate BL may not be provided.

The display apparatus DD according to an embodiment may further include a plugging layer. The plugging layer may be disposed between a display element layer DP-ED and a base substrate BL. The plugging layer may be an organic layer. The plugging layer may include at least one selected from among an acrylic resin, a silicon-based resin and an epoxy-based resin.

The display panel DP may include a base layer BS, a circuit layer DP-CL provided on the base layer BS and a display element layer DP-ED. The display element layer DP-ED may include a pixel definition layer PDL, light emitting elements ED-1, ED-2 and ED-3 disposed in the pixel definition layer PDL, and an encapsulating layer TFE disposed on the light emitting elements ED-1, ED-2 and ED-3.

The base layer BS may be a member providing a base surface where the display element layer DP-ED is disposed. The base layer BS may be a glass substrate, a metal substrate, a plastic substrate, etc. However, the present disclosure is not limited thereto, and the base layer BS may be an inorganic layer, an organic layer or a composite material layer.

In an embodiment, the circuit layer DP-CL is disposed on the base layer BS, and the circuit layer DP-CL may include multiple transistors. Each of the transistors may include a control electrode, an input electrode, and an output electrode. For example, the circuit layer DP-CL may include switching transistors and driving transistors for driving the light emitting elements ED-1, ED-2 and ED-3 of the display element layer DP-ED.

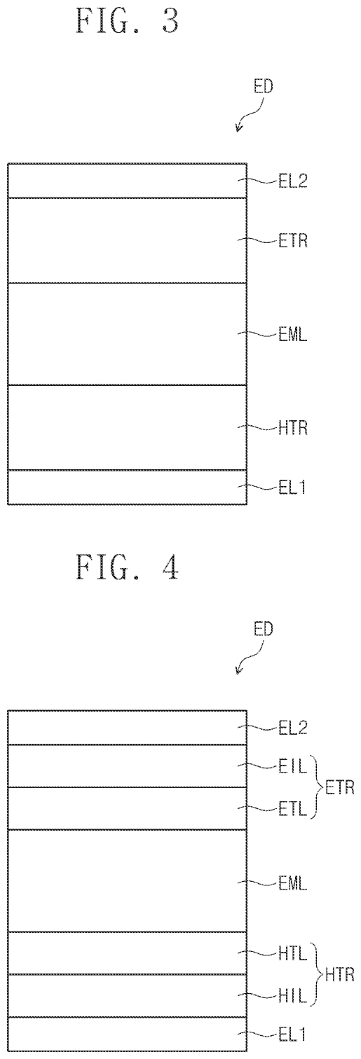

The light emitting elements ED-1, ED-2 and ED-3 may each have the structures of the light emitting elements ED of embodiments according to FIG. 3 to FIG. 6, which will be explained in more detail later. The light emitting elements ED-1, ED-2 and ED-3 may each include a first electrode EL1, a hole transport region HTR, emission layers EML-R, EML-G and EML-B, an electron transport region ETR and a second electrode EL2.

FIG. 2 shows an embodiment where the emission layers EML-R, EML-G and EML-B of light emitting elements ED-1, ED-2 and ED-3 are disposed in opening portions OH defined in a pixel definition layer PDL, and a hole transport region HTR, an electron transport region ETR and a second electrode EL2 are provided as common layers in all light emitting elements ED-1, ED-2 and ED-3. However, the present disclosure is not limited thereto. Different from FIG. 2, in an embodiment, the hole transport region HTR and the electron transport region ETR may be patterned and provided in the opening portions OH defined in the pixel definition layer PDL. For example, in an embodiment, the hole transport region HTR, the emission layers EML-R, EML-G and EML-B, and the electron transport region ETR of the light emitting elements ED-1, ED-2 and ED-3 may be patterned and provided by an ink jet printing method.

An encapsulating layer TFE may cover the light emitting elements ED-1, ED-2 and ED-3. The encapsulating layer TFE may encapsulate the display element layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to an embodiment may include at least one inorganic layer (hereinafter, encapsulating inorganic layer). In some embodiments, the encapsulating layer TFE according to an embodiment may include at least one organic layer (hereinafter, encapsulating organic layer) and at least one encapsulating inorganic layer.

The encapsulating inorganic layer protects the display element layer DP-ED from moisture/oxygen, and the encapsulating organic layer protects the display element layer DP-ED from foreign materials such as dust particles. The encapsulating inorganic layer may include silicon nitride, silicon oxy nitride, silicon oxide, titanium oxide, and/or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be disposed on the second electrode EL2 and may be disposed while filling the opening portion OH.

Referring to FIG. 1 and FIG. 2, the display apparatus DD may include a non-luminous area NPXA and luminous areas PXA-R, PXA-G and PXA-B. The luminous areas PXA-R, PXA-G and PXA-B may be areas emitting light produced from the light emitting elements ED-1, ED-2 and ED-3, respectively. The luminous areas PXA-R, PXA-G and PXA-B may be separated from each other on a plane.

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In some embodiments, in the disclosure, each of the luminous areas PXA-R, PXA-G and PXA-B may correspond to a pixel. The pixel definition layer PDL may divide the light emitting elements ED-1, ED-2 and ED-3. The emission layers EML-R, EML-G and EML-B of the light emitting elements ED-1, ED-2 and ED-3 may be disposed and divided in the opening portions OH defined in the pixel definition layer PDL.

The luminous areas PXA-R, PXA-G and PXA-B may be divided into multiple groups according to the color of light produced from the light emitting elements ED-1, ED-2 and ED-3. In the display apparatus DD of an embodiment, shown in FIG. 1 and FIG. 2, three luminous areas PXA-R, PXA-G and PXA-B respectively emitting red light, green light and blue light are illustrated as an embodiment. For example, the display apparatus DD of an embodiment may include a red luminous area PXA-R, a green luminous area PXA-G and a blue luminous area PXA-B, which are separated from each other.

In the display apparatus DD according to an embodiment, multiple light emitting elements ED-1, ED-2 and ED-3 may be to emit light having different wavelength regions. For example, in an embodiment, the display apparatus DD may include a first light emitting element ED-1 emitting red light, a second light emitting element ED-2 emitting green light, and a third light emitting element ED-3 emitting blue light. For example, each of the red luminous area PXA-R, the green luminous area PXA-G, and the blue luminous area PXA-B of the display apparatus DD may respectively correspond to the first light emitting element ED-1, the second light emitting element ED-2, and the third light emitting element ED-3.

However, the present disclosure is not limited thereto, and the first to third light emitting elements ED-1, ED-2 and ED-3 may be to emit light in substantially the same wavelength region, or at least one thereof may be to emit light in a different wavelength region. For example, all the first to third light emitting elements ED-1, ED-2 and ED-3 may be to emit blue light.

The luminous areas PXA-R, PXA-G and PXA-B in the display apparatus DD according to an embodiment may be arranged in a stripe shape. Referring to FIG. 1, multiple red luminous areas PXA-R may be arranged with each other along a second direction axis DR2, multiple green luminous areas PXA-G may be arranged with each other along the second direction axis DR2, and multiple blue luminous areas PXA-B may be arranged with each other along the second direction axis DR2. In some embodiments, the red luminous area PXA-R, the green luminous area PXA-G and the blue luminous area PXA-B may be arranged by turns (with each other) along a first directional axis DR1.

In FIG. 1 and FIG. 2, the areas of the luminous areas PXA-R, PXA-G and PXA-B are shown as being similar, but the present disclosure is not limited thereto. The areas of the luminous areas PXA-R, PXA-G and PXA-B may be different from each other according to the wavelength region of light emitted. In some embodiments, the areas of the luminous areas PXA-R, PXA-G and PXA-B may refer to areas in a plan view (e.g., on a plane defined by the first directional axis DR1 and the second directional axis DR2).

In some embodiments, the arrangement type or kind of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in one or more suitable combinations according to the properties of display quality required for the display apparatus DD. For example, the arrangement pattern of the luminous areas PXA-R, PXA-G and PXA-B may be a PENTILE™ arrangement, or a Diamond Pixel™ arrangement. PENTILE® and Diamond Pixel™ are both trademarks of Samsung Display Co., Ltd.

In some embodiments, the areas of the luminous areas PXA-R, PXA-G and PXA-B may be different from each other. For example, in an embodiment, the area of the green luminous area PXA-G may be smaller than the area of the blue luminous area PXA-B, but the present disclosure is not limited thereto.

Hereinafter, FIG. 3 to FIG. 6 are cross-sectional views schematically showing light emitting elements according to embodiments. The light emitting element ED according to an embodiment may include a first electrode EL1, a second electrode EL2 oppositely disposed to the first electrode EL1, and at least one functional layer disposed between the first electrode EL1 and the second electrode EL2. The light emitting element ED of an embodiment may include an amine compound of an embodiment, which will be explained in more detail later, in the at least one functional layer.

The light emitting element ED may include a hole transport region HTR, an emission layer EML, an electron transport region ETR, and/or the like, stacked in the stated order, as the at least one functional layer. Referring to FIG. 3, the light emitting element ED of an embodiment may include a first electrode EL1, a hole transport region HTR, an emission layer EML, an electron transport region ETR, and a second electrode EL2, stacked in the stated order.

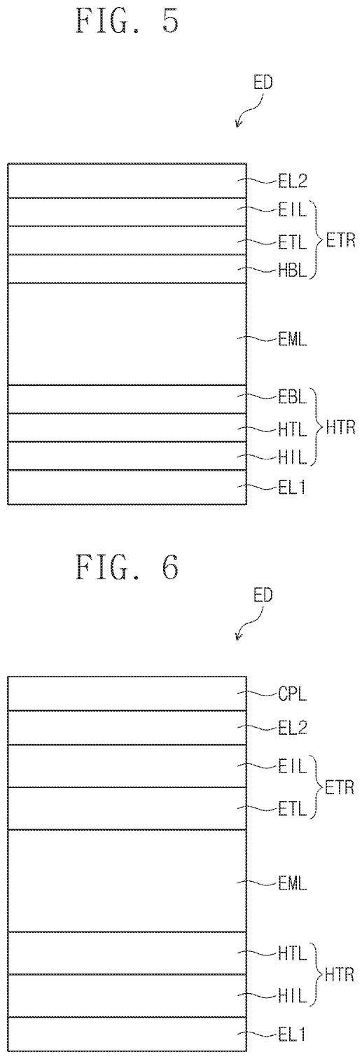

When compared with FIG. 3, FIG. 4 shows the cross-sectional view of a light emitting element ED of an embodiment, wherein a hole transport region HTR includes a hole injection layer HIL and a hole transport layer HTL, and an electron transport region ETR includes an electron injection layer EIL and an electron transport layer ETL. In addition, when compared with FIG. 3, FIG. 5 shows the cross-sectional view of a light emitting element ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared with FIG. 4, FIG. 6 shows the cross-sectional view of a light emitting element ED of an embodiment, including a capping layer CPL disposed on the second electrode EL2.

The light emitting element ED of an embodiment may include an amine compound of an embodiment, which will be explained in more detail later, in a hole transport region HTR. The light emitting element ED of an embodiment may include an amine compound of an embodiment in at least one selected from among the hole injection layer HIL, hole transport layer HTL, and electron blocking layer EBL of the hole transport region HTR. For example, the light emitting element ED of an embodiment may include an amine compound of an embodiment in the hole transport layer HTL or the electron blocking layer EBL of the hole transport region HTR.

In the light emitting element ED according to an embodiment, the first electrode EL1 has conductivity (e.g., is a conductor). The first electrode EL1 may be formed utilizing a metal material, a metal alloy or a conductive compound. The first electrode EL1 may be an anode or a cathode. However, the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn and Zn, compounds of two or more selected therefrom, mixtures of two or more selected therefrom, and/or oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, one or more compounds thereof, or one or more mixtures thereof (for example, a mixture of Ag and Mg). In some embodiments, the first electrode EL1 may have a structure including multiple layers including a reflective layer or a transflective layer formed utilizing the above materials, and a transmissive conductive layer formed utilizing ITO, IZO, ZnO, and/or ITZO. For example, the first electrode EL1 may have a three-layer structure of ITO/Ag/ITO. However, the present disclosure is not limited thereto. The first electrode EL1 may include the above-described metal materials, combinations of two or more metal materials selected from the above-described metal materials, or oxides of the above-described metal materials. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may have a single layer formed utilizing a single material, a single layer formed utilizing multiple different materials, or a multilayer structure including multiple layers formed utilizing multiple different materials.

The hole transport region HTR may include at least one of a hole injection layer HIL, a hole transport layer HTL, or an electron blocking layer EBL. In some embodiments, the hole transport region HTR may have a stacked structure of a hole injection layer HIL and a hole transport layer HTL, or a stacked structure of a hole injection layer HIL, a hole transport layer HTL, and an electron blocking layer EBL.

In some embodiments, the hole transport region HTR may have the structure of a single layer of a hole injection layer HIL or a hole transport layer HTL, and may have a structure of a single layer formed utilizing a hole injection material and a hole transport material. In an embodiment, the hole transport region HTR may have a structure of a single layer formed utilizing multiple different materials, or a structure stacked from the first electrode EL1 of hole injection layer HIL/hole transport layer HTL, hole injection layer HIL/hole transport layer HTL/buffer layer, hole injection layer HIL/hole transport layer HTL/electron blocking layer EBL, hole injection layer HIL/buffer layer, or hole transport layer HTL/buffer layer, but the present disclosure is not limited thereto.

The thickness of the hole transport region HTR may be, for example, about 50 Å to about 15,000 Å. The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of an embodiment may include the amine compound of an embodiment in the hole transport region HTR. In the light emitting element ED of an embodiment, the amine compound of an embodiment may be included in at least one of a hole injection layer HIL, a hole transport layer HTL, or an electron blocking layer EBL. For example, in the light emitting element ED of an embodiment, a hole transport layer HTL or an electron blocking layer EBL may include the amine compound of an embodiment, represented by Formula 1.

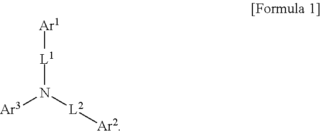

In Formula 1, L¹ and L² may each independently be a direct linkage, a substituted or unsubstituted arylene group having 6 to 30 ring-forming carbon atoms, or a substituted or unsubstituted heteroarylene group having 5 to 30 ring-forming carbon atoms. In some embodiments, L¹ and L² may each independently be a direct linkage, or a substituted or unsubstituted arylene group having 6 to 20 ring-forming carbon atoms. For example, L¹ and L² may each independently be a substituted or unsubstituted phenylene group, a substituted or unsubstituted biphenylene group, a substituted or unsubstituted terphenylene group, or a substituted or unsubstituted naphthylene group. However, the present disclosure is not limited thereto.

In Formula 1, Ar¹ and Ar² may each independently be a substituted or unsubstituted aryl group having 6 to 30 ring-forming carbon atoms, or a substituted or unsubstituted heteroaryl group having 5 to 30 ring-forming carbon atoms. For example, Ar¹ and Ar² may each independently be a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, or a substituted or unsubstituted fluorenyl group. In some embodiments, Ar¹ and Ar² may each independently be a ring group having a substituted or unsubstituted dibenzoheterole skeleton such as a substituted or unsubstituted dibenzofuran group, a substituted or unsubstituted dibenzothiophene group, a substituted or unsubstituted naphthobenzofuran group, and a substituted or unsubstituted benzonaphthothiophene group. However, the present disclosure is not limited thereto.

In some embodiments, when Ar¹ or Ar² is a substituent having a fluorene skeleton, and when the nitrogen atom, L¹, or L² of Formula 1 is combined at position 9 of the fluorene

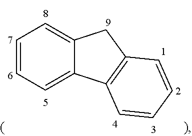 [SMILES: c1ccc2c(c1)Cc1ccccc1-2]

element properties may be deteriorated. Accordingly, when Ar¹ or Ar² in Formula 1 is a substituent having a fluorene skeleton, the combination position of the substituent having the fluorene skeleton may be limited. For example, in the amine compound of an embodiment, represented by Formula 1, when Ar¹ or Ar² is a substituted or unsubstituted fluorenyl group, any one selected from among two benzene rings forming the 3-ring fluorenyl group may be bonded (e.g., directly) with L¹ or L², or may be bonded (e.g., directly) with the nitrogen atom of Formula 1.

In Formula 1, L¹-Ar¹ and L²-Ar² may not include (e.g., may exclude) a carbazole group (e.g., L¹-Ar¹ and L²-Ar² may not include any carbazole group) so as to achieve good or suitable carrier balance in a molecule. In some embodiments, L¹-Ar¹ and L²-Ar² may not include (e.g., may exclude) a silyl group (e.g., L¹-Ar¹ and L²-Ar² may not include any silyl group) which reduces intermolecular interaction due to its large steric volume.

In Formula 1, Ar³ may be different from Ar¹ and Ar². Ar³ may be represented by Formula 2.

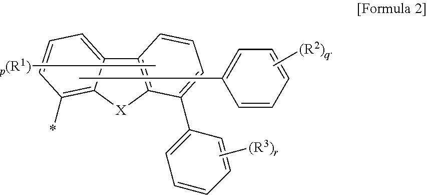

In Formula 2, X may be O or S. For example, Formula 2 may include a dibenzofuran skeleton or a dibenzothiophene skeleton as a basic skeleton. In some embodiments, in Formula 2, *- is a position combined with the nitrogen atom of Formula 1.

In Formula 2, R¹ may be a hydrogen atom, a deuterium atom, a halogen atom, or a substituted or unsubstituted alkyl group having 1 to 20 carbon atoms. For example, R¹ may be a hydrogen atom, but the present disclosure is not limited thereto.

In Formula 2, R² and R³ may each independently be a hydrogen atom, a deuterium atom, a halogen atom, a substituted or unsubstituted alkyl group having 1 to 20 carbon atoms, or a substituted or unsubstituted aryl group having 6 to 30 ring-forming carbon atoms, or combined with an adjacent group to form a hydrocarbon ring. In some embodiments, R² and R³ may each independently be a hydrogen atom, a deuterium atom, a substituted or unsubstituted aryl group having 6 to 15 ring-forming carbon atoms, or combined with an adjacent group to form an aromatic hydrocarbon ring. For example, each of R² and R³ may be a hydrogen atom, a deuterium atom, or a substituted or unsubstituted phenyl group, or R² and/or R³ may be combined with a phenyl group substituent to form a substituted or unsubstituted naphthyl group. However, the present disclosure is not limited thereto.

In Formula 2, “p”, “q” and “r” may each independently be an integer of 0 to 5. A case where “p” is 0 may be the same as a case where “p” is 1 and R¹ is a hydrogen atom. A case where “q” is 0 may be the same as a case where “q” is 1 and R² is a hydrogen atom, and a case where “r” is 0 may be the same as a case where “r” is 1 and R³ is a hydrogen atom.

In the amine compound of an embodiment, represented by Formula 1, at least one selected from among L¹, L², and Ar¹ to Ar³ may be a substituent including a deuterium atom. For example, the amine compound of an embodiment may include at least one deuterium atom as a substituent.

In an embodiment, Formula 2 may be represented by any one selected from among Formula 2-1 to Formula 2-4. Each of Formula 2-1 to Formula 2-4 corresponds to Formula 2 where the combination position of substituted or unsubstituted phenyl group with R² is embodied.

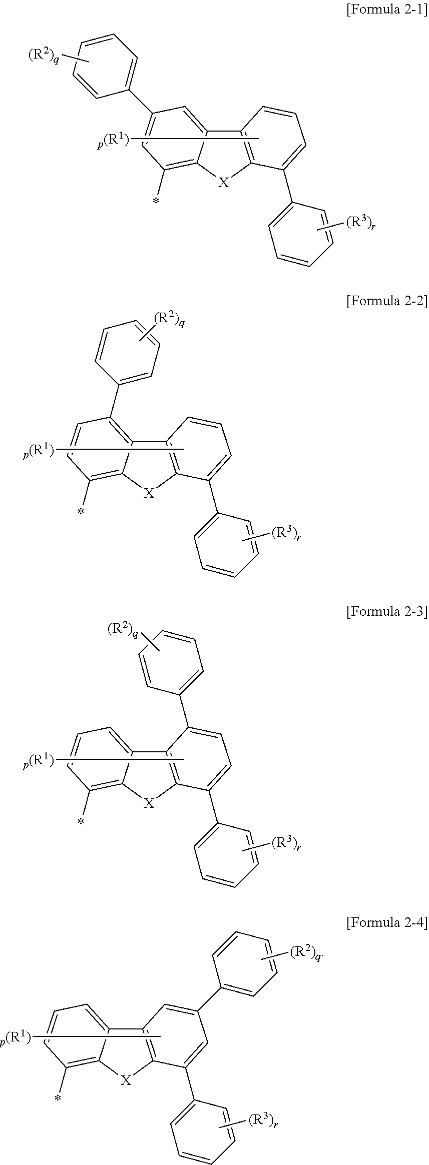

In Formula 2-1 to Formula 2-4, the same explanation on X, R¹, R², R³, “p” “q” and “r” explained in Formula 2 may be applied. Meanwhile, in Formula 2-1 to Formula 2-4, *- is a position connected with the nitrogen atom in Formula 1.

In an embodiment, Formula 1 may be represented by Formula 3. Formula 3 is an embodiment of Formula 1 combined with Formula 2 in which R¹ is a hydrogen atom.

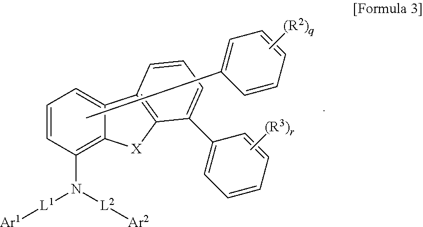

In Formula 3, the same explanation on X, L¹, L², Ar¹, Ar², R², R³, “q” and “r” explained in Formula 1 and Formula 2 may be applied.

In an embodiment, the amine compound represented by Formula 3 may be represented by any one selected from among Formula 3-1 to Formula 3-4.

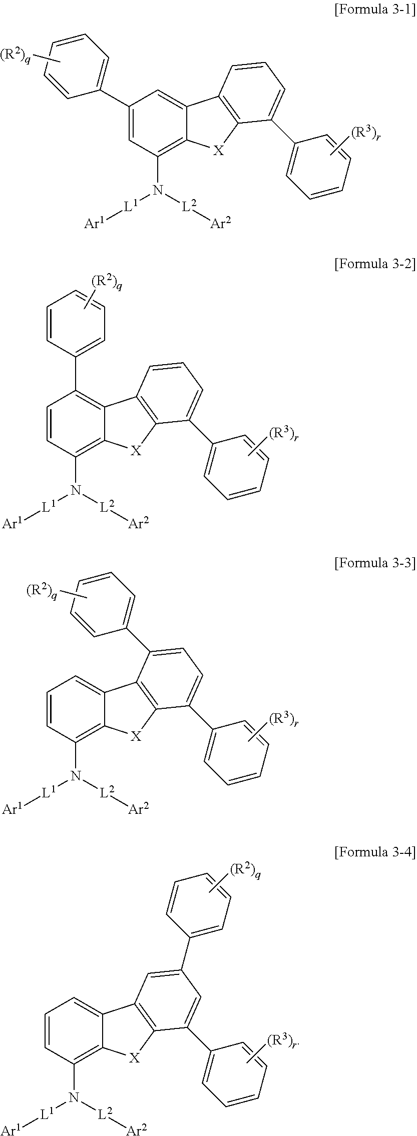

In Formula 3-1 to Formula 3-4, the same explanation on X, L¹, L², Ar¹, Ar², R², R³, “q” and “r” explained in Formula 1 and Formula 2 may be applied.

The amine compound of an embodiment may be represented by Formula 4. Formula 4 is an embodiment of the amine compound of Formula 1 combined with Formula 2 where R¹, R² and R³ are each a hydrogen atom. In addition, Formula 4 may be an embodiment of Formula 3 where R¹, R² and R³ are each a hydrogen atom.

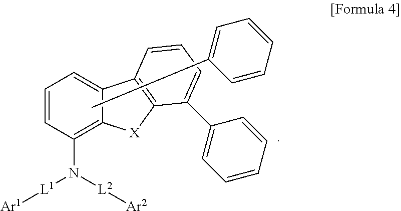

In Formula 4, the same explanation on X, L¹, L², Ar¹, and Ar² explained in Formula 1 may be applied.

The amine compound of an embodiment, represented by Formula 1 may be represented by any one selected from among the compounds in Compound Group 1. The hole transport region HTR of the light emitting element ED of an embodiment may include at least one selected from among the amine compounds disclosed in Compound Group 1. In Compound Group 1, D is a deuterium atom.

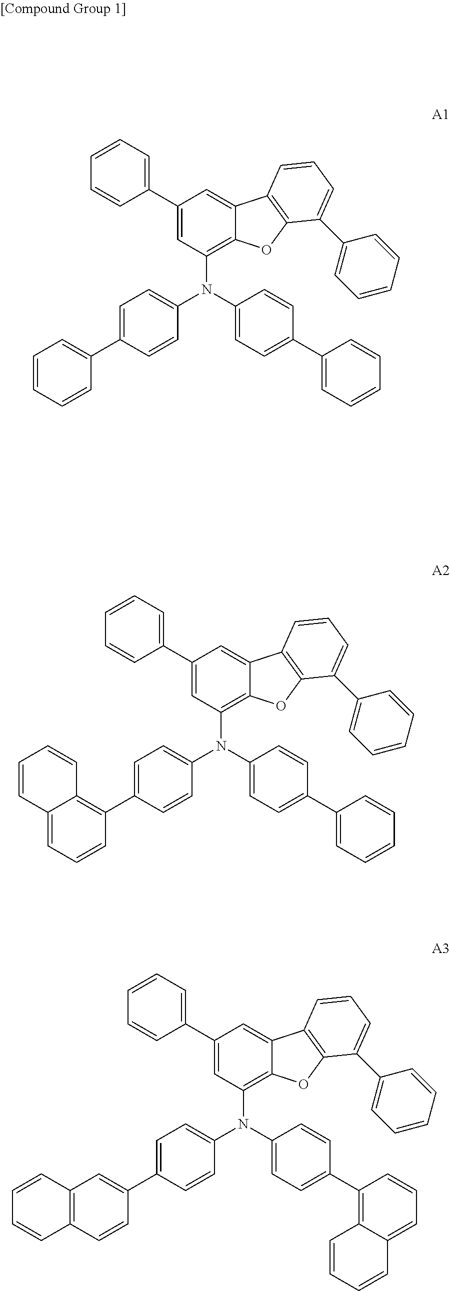 [SMILES: c1ccc(-c2ccc(N(c3ccc(-c4ccccc4)cc3)c3cc(-c4ccccc4)cc4c3oc3c(-c5ccccc5)cccc34)cc2)cc1 ; c1ccc(-c2ccc(N(c3ccc(-c4cccc5ccccc45)cc3)c3cc(-c4ccccc4)cc4c3oc3c(-c5ccccc5)cccc34)cc2)cc1 ; c1ccc(-c2cc(N(c3ccc(-c4ccc5ccccc5c4)cc3)c3ccc(-c4cccc5ccccc45)cc3)c3oc4c(-c5ccccc5)cccc4c3c2)cc1]

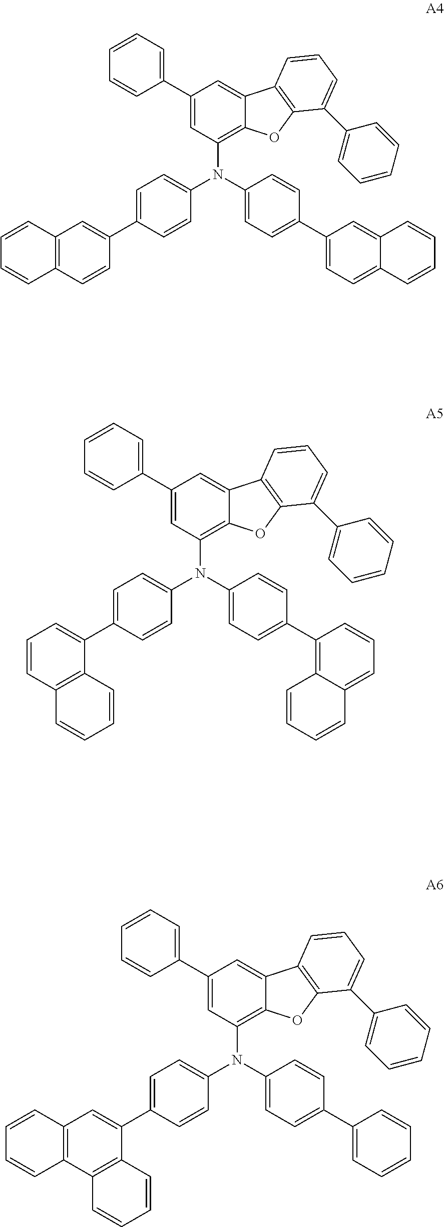 [SMILES: c1ccc(-c2cc(N(c3ccc(-c4ccc5ccccc5c4)cc3)c3ccc(-c4ccc5ccccc5c4)cc3)c3oc4c(-c5ccccc5)cccc4c3c2)cc1 ; c1ccc(-c2cc(N(c3ccc(-c4cccc5ccccc45)cc3)c3ccc(-c4cccc5ccccc45)cc3)c3oc4c(-c5ccccc5)cccc4c3c2)cc1 ; c1ccc(-c2ccc(N(c3ccc(-c4cc5ccccc5c5ccccc45)cc3)c3cc(-c4ccccc4)cc4c3oc3c(-c5ccccc5)cccc34)cc2)cc1]

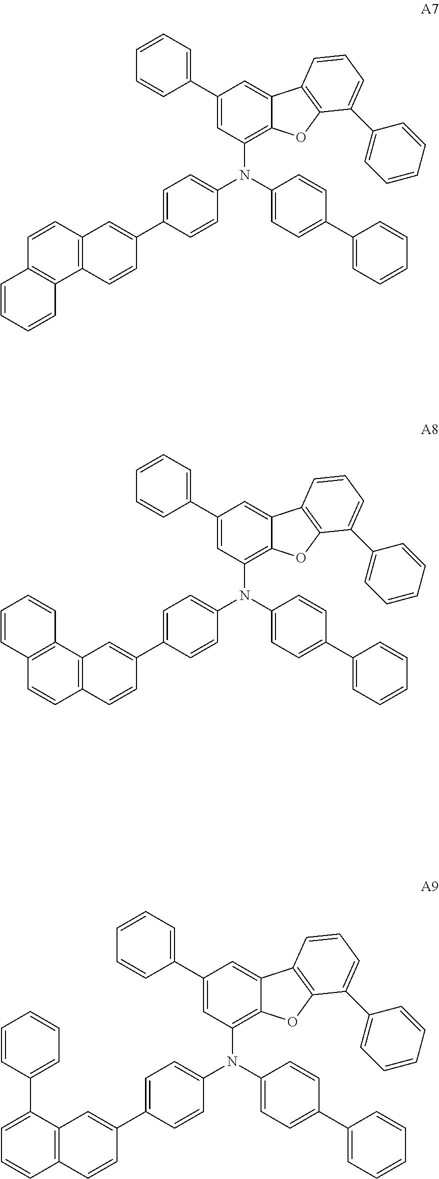 [SMILES: c1ccc(-c2ccc(N(c3ccc(-c4ccc5c(ccc6ccccc65)c4)cc3)c3cc(-c4ccccc4)cc4c3oc3c(-c5ccccc5)cccc34)cc2)cc1 ; c1ccc(-c2ccc(N(c3ccc(-c4ccc5ccc6ccccc6c5c4)cc3)c3cc(-c4ccccc4)cc4c3oc3c(-c5ccccc5)cccc34)cc2)cc1 ; c1ccc(-c2ccc(N(c3ccc(-c4ccc5cccc(-c6ccccc6)c5c4)cc3)c3cc(-c4ccccc4)cc4c3oc3c(-c5ccccc5)cccc34)cc2)cc1]

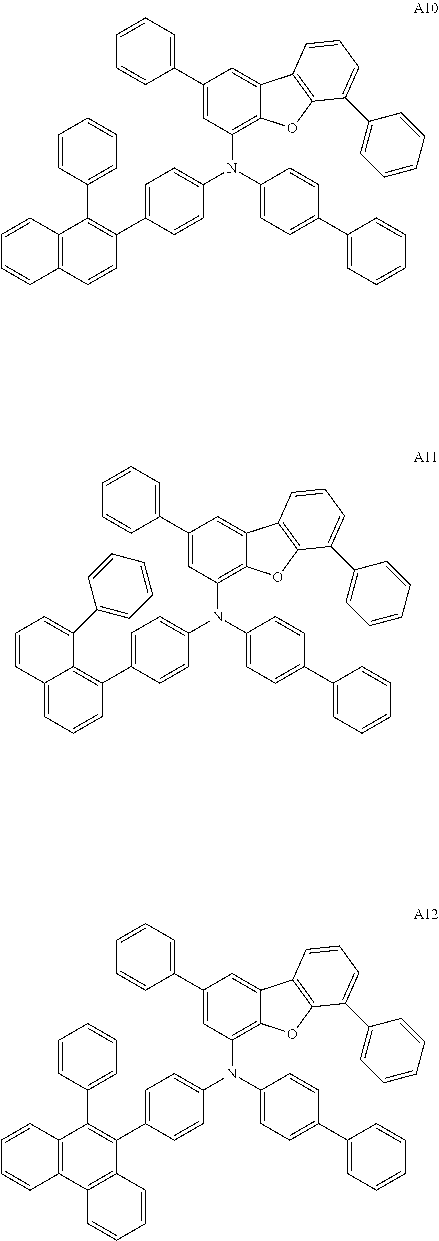 [SMILES: c1ccc(-c2ccc(N(c3ccc(-c4ccc5ccccc5c4-c4ccccc4)cc3)c3cc(-c4ccccc4)cc4c3oc3c(-c5ccccc5)cccc34)cc2)cc1 ; c1ccc(-c2ccc(N(c3ccc(-c4cccc5cccc(-c6ccccc6)c45)cc3)c3cc(-c4ccccc4)cc4c3oc3c(-c5ccccc5)cccc34)cc2)cc1 ; c1ccc(-c2ccc(N(c3ccc(-c4c(-c5ccccc5)c5ccccc5c5ccccc45)cc3)c3cc(-c4ccccc4)cc4c3oc3c(-c5ccccc5)cccc34)cc2)cc1]

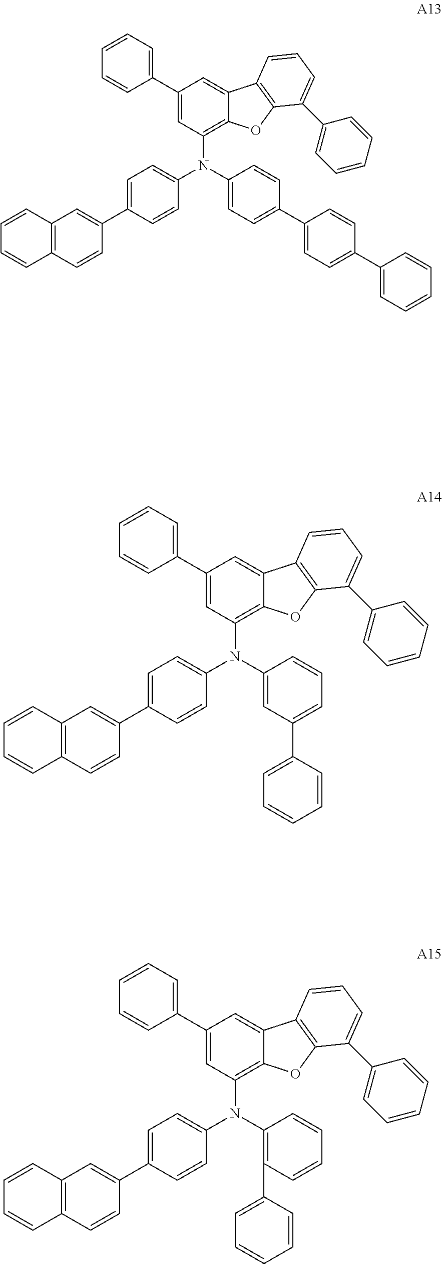 [SMILES: c1ccc(-c2ccc(-c3ccc(N(c4ccc(-c5ccc6ccccc6c5)cc4)c4cc(-c5ccccc5)cc5c4oc4c(-c6ccccc6)cccc45)cc3)cc2)cc1 ; c1ccc(-c2cccc(N(c3ccc(-c4ccc5ccccc5c4)cc3)c3cc(-c4ccccc4)cc4c3oc3c(-c5ccccc5)cccc34)c2)cc1 ; c1ccc(-c2cc(N(c3ccc(-c4ccc5ccccc5c4)cc3)c3ccccc3-c3ccccc3)c3oc4c(-c5ccccc5)cccc4c3c2)cc1]

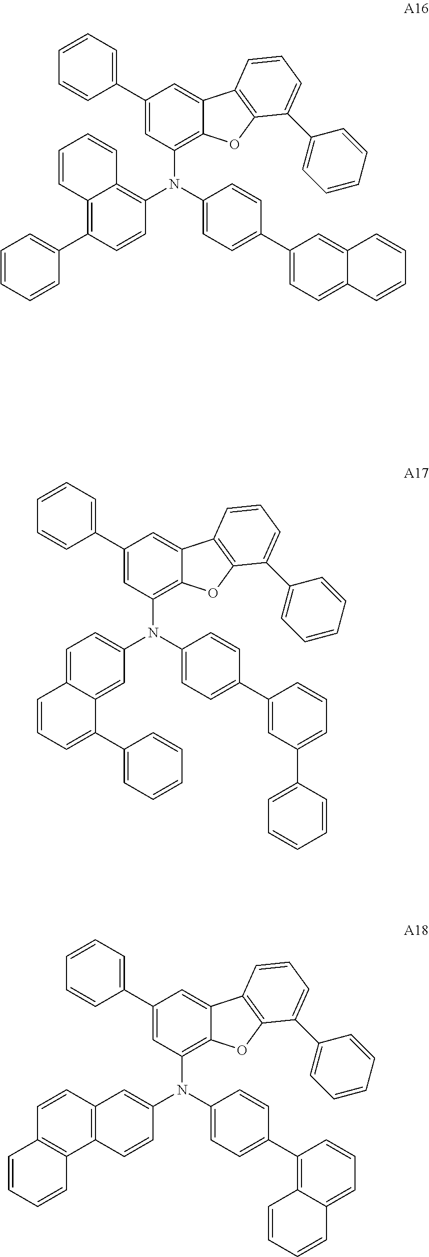 [SMILES: c1ccc(-c2cc(N(c3ccc(-c4ccc5ccccc5c4)cc3)c3ccc(-c4ccccc4)c4ccccc34)c3oc4c(-c5ccccc5)cccc4c3c2)cc1 ; c1ccc(-c2cccc(-c3ccc(N(c4ccc5cccc(-c6ccccc6)c5c4)c4cc(-c5ccccc5)cc5c4oc4c(-c6ccccc6)cccc45)cc3)c2)cc1 ; c1ccc(-c2cc(N(c3ccc(-c4cccc5ccccc45)cc3)c3ccc4c(ccc5ccccc54)c3)c3oc4c(-c5ccccc5)cccc4c3c2)cc1]

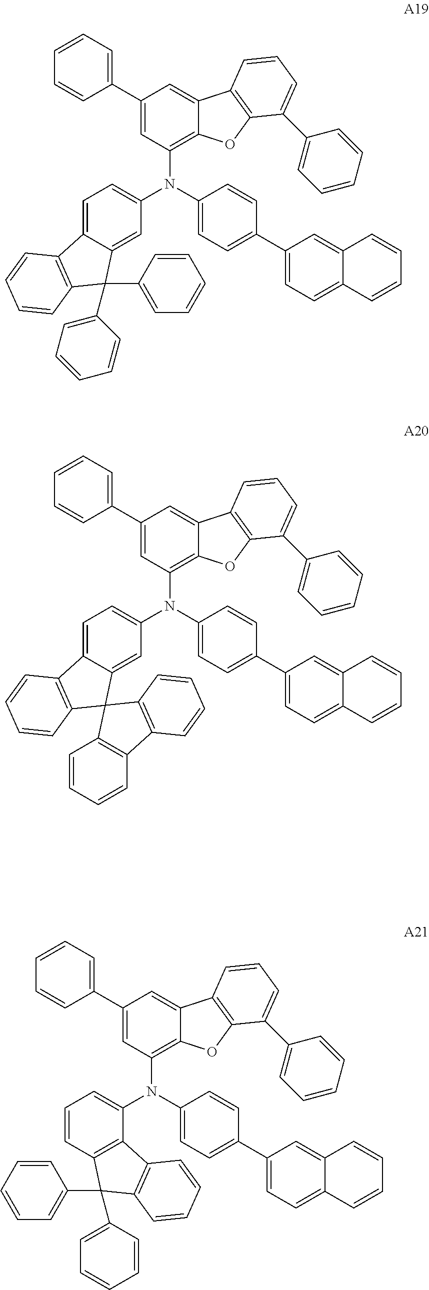 [SMILES: c1ccc(-c2cc(N(c3ccc(-c4ccc5ccccc5c4)cc3)c3ccc4c(c3)C(c3ccccc3)(c3ccccc3)c3ccccc3-4)c3oc4c(-c5ccccc5)cccc4c3c2)cc1 ; c1ccc(-c2cc(N(c3ccc(-c4ccc5ccccc5c4)cc3)c3ccc4c(c3)C3(c5ccccc5-c5ccccc53)c3ccccc3-4)c3oc4c(-c5ccccc5)cccc4c3c2)cc1 ; c1ccc(-c2cc(N(c3ccc(-c4ccc5ccccc5c4)cc3)c3cccc4c3-c3ccccc3C4(c3ccccc3)c3ccccc3)c3oc4c(-c5ccccc5)cccc4c3c2)cc1]

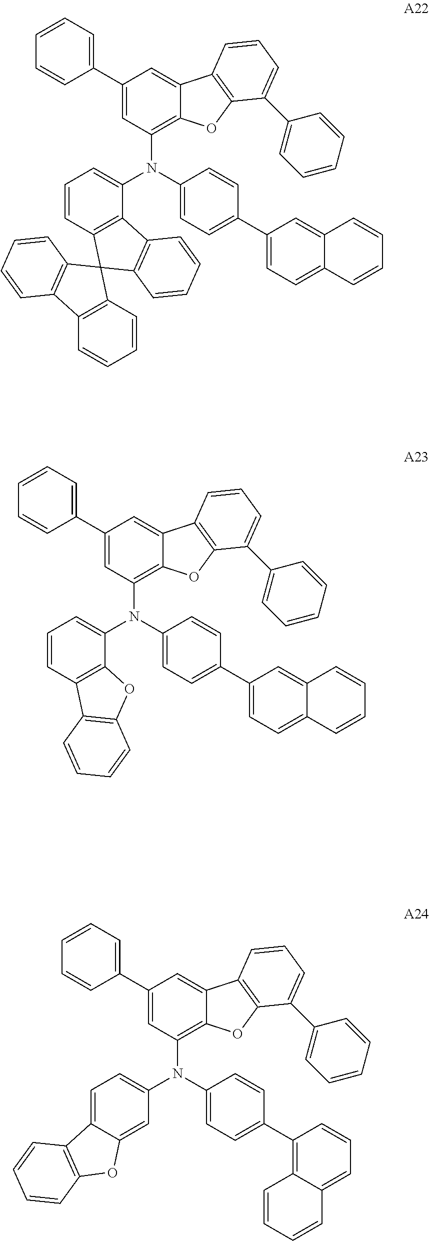 [SMILES: c1ccc(-c2cc(N(c3ccc(-c4ccc5ccccc5c4)cc3)c3cccc4c3-c3ccccc3C43c4ccccc4-c4ccccc43)c3oc4c(-c5ccccc5)cccc4c3c2)cc1 ; c1ccc(-c2cc(N(c3ccc(-c4ccc5ccccc5c4)cc3)c3cccc4c3oc3ccccc34)c3oc4c(-c5ccccc5)cccc4c3c2)cc1 ; c1ccc(-c2cc(N(c3ccc(-c4cccc5ccccc45)cc3)c3ccc4c(c3)oc3ccccc34)c3oc4c(-c5ccccc5)cccc4c3c2)cc1]

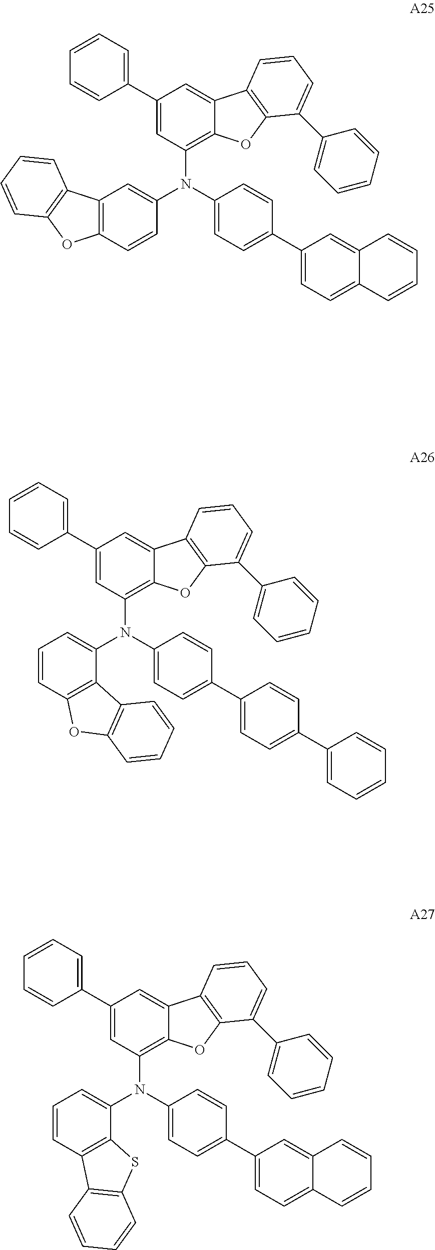 [SMILES: c1ccc(-c2cc(N(c3ccc(-c4ccc5ccccc5c4)cc3)c3ccc4oc5ccccc5c4c3)c3oc4c(-c5ccccc5)cccc4c3c2)cc1 ; c1ccc(-c2ccc(-c3ccc(N(c4cc(-c5ccccc5)cc5c4oc4c(-c6ccccc6)cccc45)c4cccc5oc6ccccc6c45)cc3)cc2)cc1 ; c1ccc(-c2cc(N(c3ccc(-c4ccc5ccccc5c4)cc3)c3cccc4c3sc3ccccc34)c3oc4c(-c5ccccc5)cccc4c3c2)cc1]

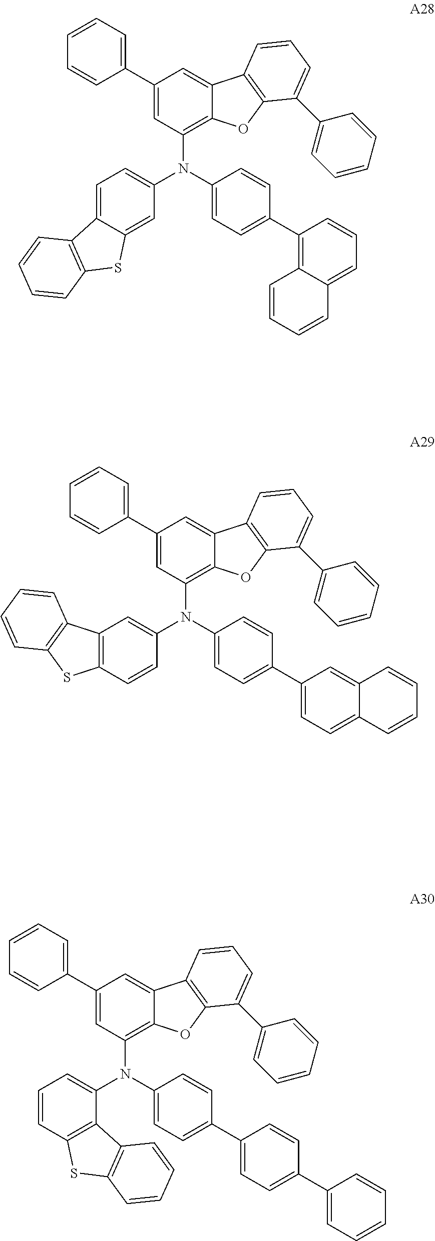 [SMILES: c1ccc(-c2cc(N(c3ccc(-c4cccc5ccccc45)cc3)c3ccc4c(c3)sc3ccccc34)c3oc4c(-c5ccccc5)cccc4c3c2)cc1 ; c1ccc(-c2cc(N(c3ccc(-c4ccc5ccccc5c4)cc3)c3ccc4sc5ccccc5c4c3)c3oc4c(-c5ccccc5)cccc4c3c2)cc1 ; c1ccc(-c2ccc(-c3ccc(N(c4cc(-c5ccccc5)cc5c4oc4c(-c6ccccc6)cccc45)c4cccc5sc6ccccc6c45)cc3)cc2)cc1]

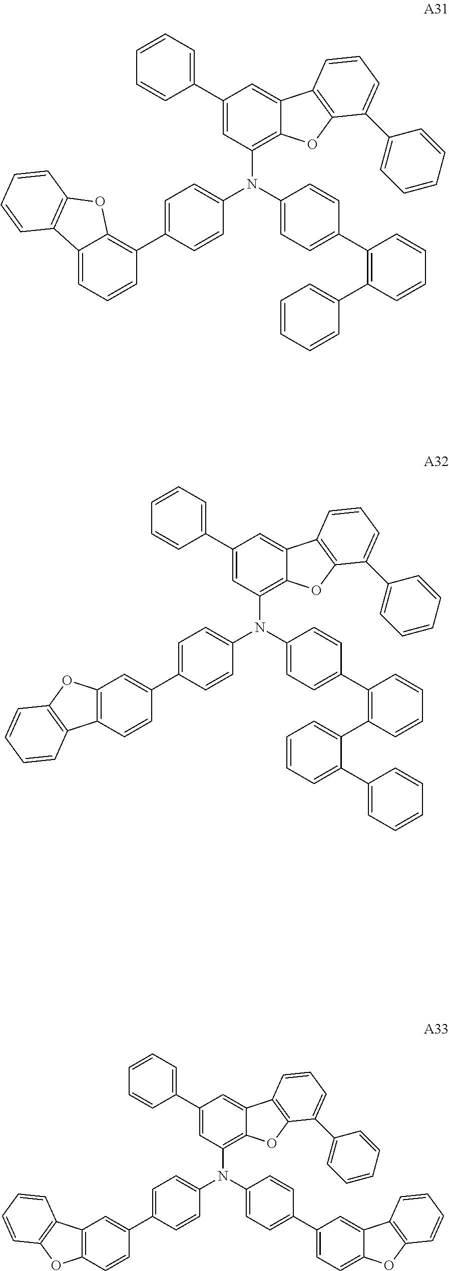 [SMILES: c1ccc(-c2cc(N(c3ccc(-c4ccccc4-c4ccccc4)cc3)c3ccc(-c4cccc5c4oc4ccccc45)cc3)c3oc4c(-c5ccccc5)cccc4c3c2)cc1 ; c1ccc(-c2cc(N(c3ccc(-c4ccc5c(c4)oc4ccccc45)cc3)c3ccc(-c4ccccc4-c4ccccc4-c4ccccc4)cc3)c3oc4c(-c5ccccc5)cccc4c3c2)cc1 ; c1ccc(-c2cc(N(c3ccc(-c4ccc5oc6ccccc6c5c4)cc3)c3ccc(-c4ccc5oc6ccccc6c5c4)cc3)c3oc4c(-c5ccccc5)cccc4c3c2)cc1]

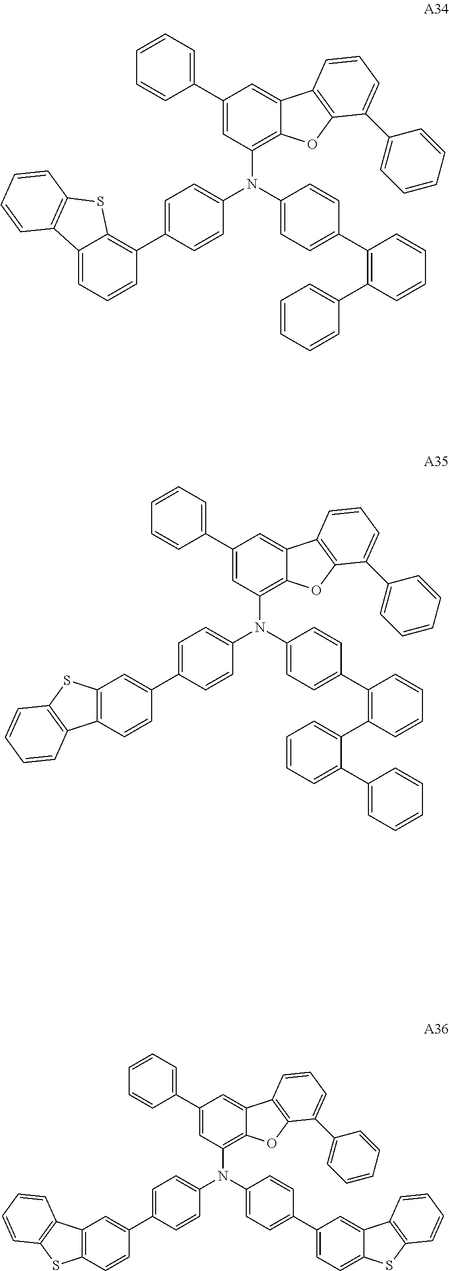 [SMILES: c1ccc(-c2cc(N(c3ccc(-c4ccccc4-c4ccccc4)cc3)c3ccc(-c4cccc5c4sc4ccccc45)cc3)c3oc4c(-c5ccccc5)cccc4c3c2)cc1 ; c1ccc(-c2cc(N(c3ccc(-c4ccc5c(c4)sc4ccccc45)cc3)c3ccc(-c4ccccc4-c4ccccc4-c4ccccc4)cc3)c3oc4c(-c5ccccc5)cccc4c3c2)cc1 ; c1ccc(-c2cc(N(c3ccc(-c4ccc5sc6ccccc6c5c4)cc3)c3ccc(-c4ccc5sc6ccccc6c5c4)cc3)c3oc4c(-c5ccccc5)cccc4c3c2)cc1]

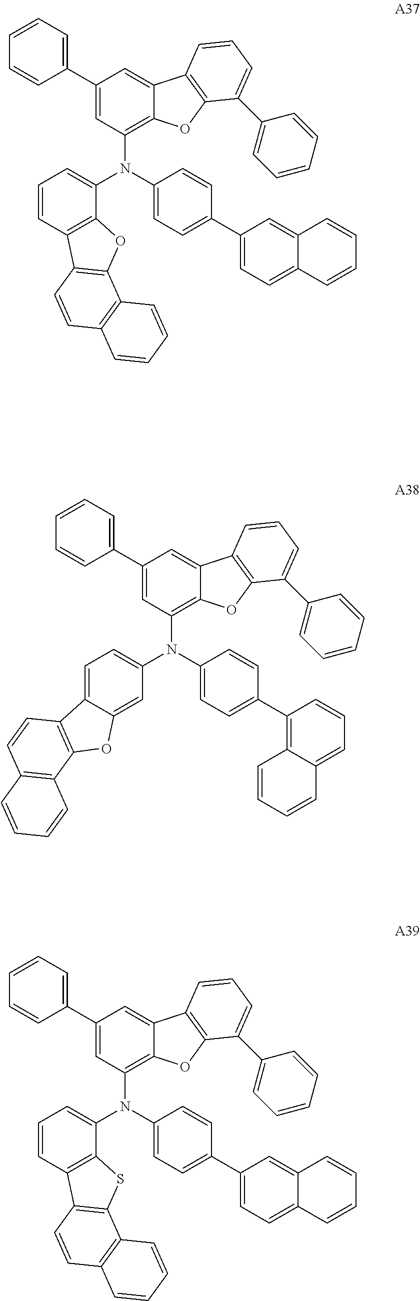 [SMILES: c1ccc(-c2cc(N(c3ccc(-c4ccc5ccccc5c4)cc3)c3cccc4c3oc3c5ccccc5ccc43)c3oc4c(-c5ccccc5)cccc4c3c2)cc1 ; c1ccc(-c2cc(N(c3ccc(-c4cccc5ccccc45)cc3)c3ccc4c(c3)oc3c5ccccc5ccc43)c3oc4c(-c5ccccc5)cccc4c3c2)cc1 ; c1ccc(-c2cc(N(c3ccc(-c4ccc5ccccc5c4)cc3)c3cccc4c3sc3c5ccccc5ccc43)c3oc4c(-c5ccccc5)cccc4c3c2)cc1]

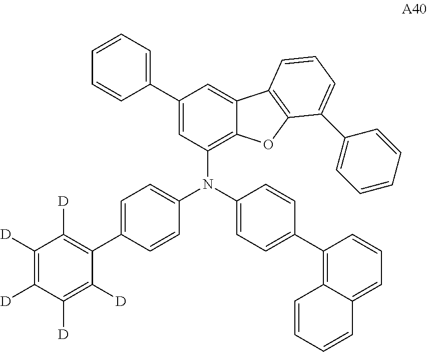 [SMILES: [2H]c1c([2H])c([2H])c(-c2ccc(N(c3ccc(-c4cccc5ccccc45)cc3)c3cc(-c4ccccc4)cc4c3oc3c(-c5ccccc5)cccc34)cc2)c([2H])c1[2H]]

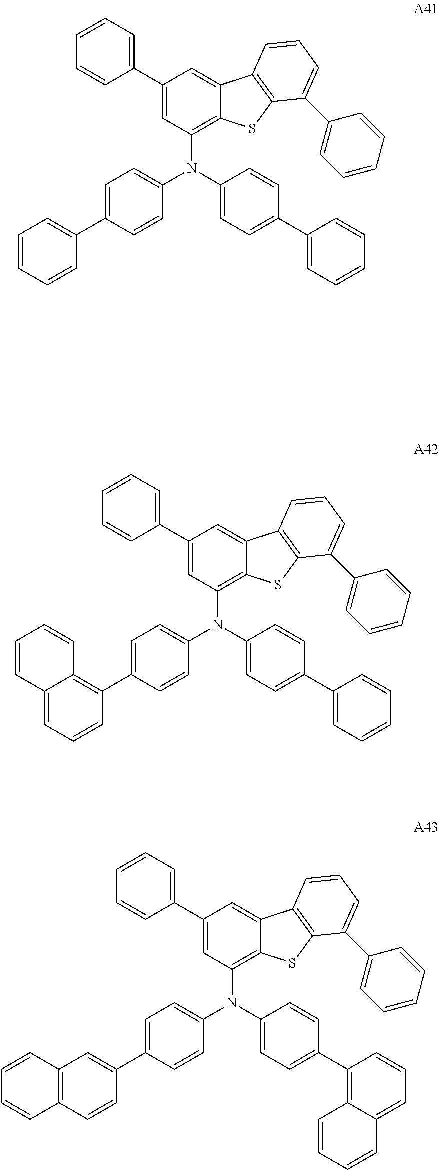 [SMILES: c1ccc(-c2ccc(N(c3ccc(-c4ccccc4)cc3)c3cc(-c4ccccc4)cc4c3sc3c(-c5ccccc5)cccc34)cc2)cc1 ; c1ccc(-c2ccc(N(c3ccc(-c4cccc5ccccc45)cc3)c3cc(-c4ccccc4)cc4c3sc3c(-c5ccccc5)cccc34)cc2)cc1 ; c1ccc(-c2cc(N(c3ccc(-c4ccc5ccccc5c4)cc3)c3ccc(-c4cccc5ccccc45)cc3)c3sc4c(-c5ccccc5)cccc4c3c2)cc1]

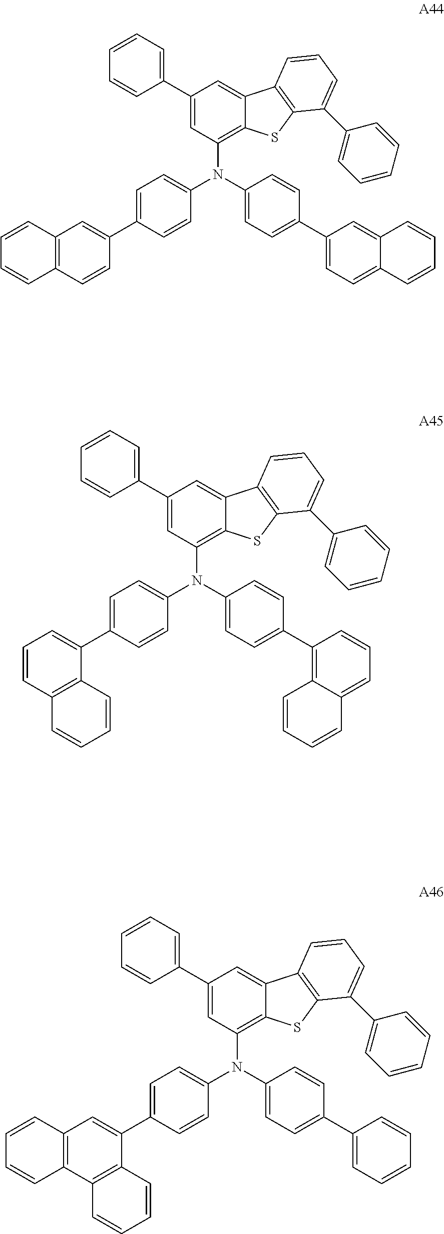 [SMILES: c1ccc(-c2cc(N(c3ccc(-c4ccc5ccccc5c4)cc3)c3ccc(-c4ccc5ccccc5c4)cc3)c3sc4c(-c5ccccc5)cccc4c3c2)cc1 ; c1ccc(-c2cc(N(c3ccc(-c4cccc5ccccc45)cc3)c3ccc(-c4cccc5ccccc45)cc3)c3sc4c(-c5ccccc5)cccc4c3c2)cc1 ; c1ccc(-c2ccc(N(c3ccc(-c4cc5ccccc5c5ccccc45)cc3)c3cc(-c4ccccc4)cc4c3sc3c(-c5ccccc5)cccc34)cc2)cc1]

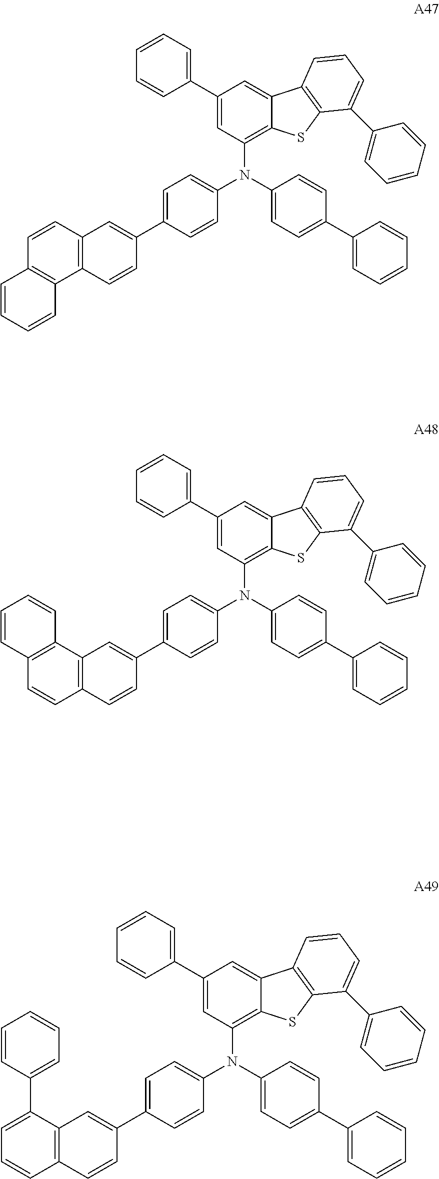 [SMILES: c1ccc(-c2ccc(N(c3ccc(-c4ccc5c(ccc6ccccc65)c4)cc3)c3cc(-c4ccccc4)cc4c3sc3c(-c5ccccc5)cccc34)cc2)cc1 ; c1ccc(-c2ccc(N(c3ccc(-c4ccc5ccc6ccccc6c5c4)cc3)c3cc(-c4ccccc4)cc4c3sc3c(-c5ccccc5)cccc34)cc2)cc1 ; c1ccc(-c2ccc(N(c3ccc(-c4ccc5cccc(-c6ccccc6)c5c4)cc3)c3cc(-c4ccccc4)cc4c3sc3c(-c5ccccc5)cccc34)cc2)cc1]

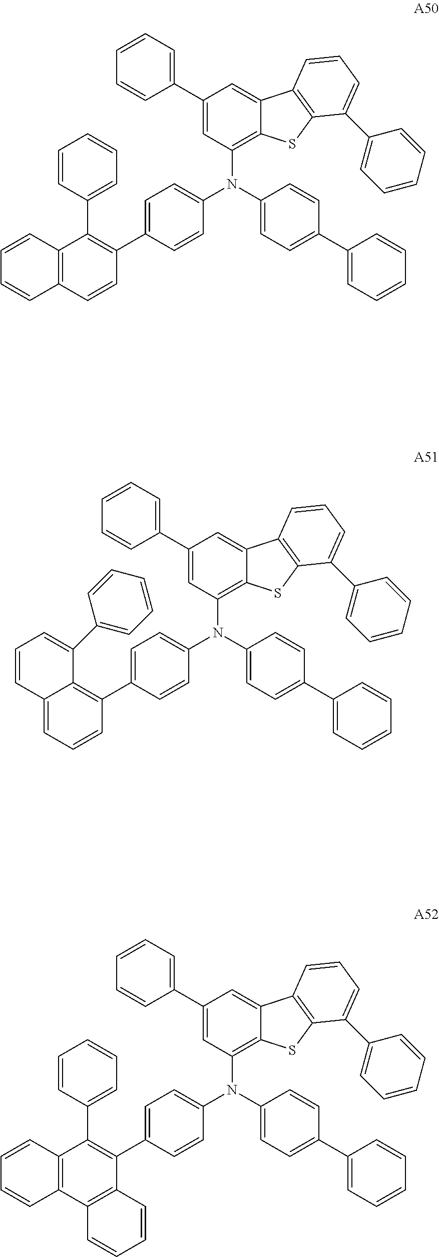 [SMILES: c1ccc(-c2ccc(N(c3ccc(-c4ccc5ccccc5c4-c4ccccc4)cc3)c3cc(-c4ccccc4)cc4c3sc3c(-c5ccccc5)cccc34)cc2)cc1 ; c1ccc(-c2ccc(N(c3ccc(-c4cccc5cccc(-c6ccccc6)c45)cc3)c3cc(-c4ccccc4)cc4c3sc3c(-c5ccccc5)cccc34)cc2)cc1 ; c1ccc(-c2ccc(N(c3ccc(-c4c(-c5ccccc5)c5ccccc5c5ccccc45)cc3)c3cc(-c4ccccc4)cc4c3sc3c(-c5ccccc5)cccc34)cc2)cc1]

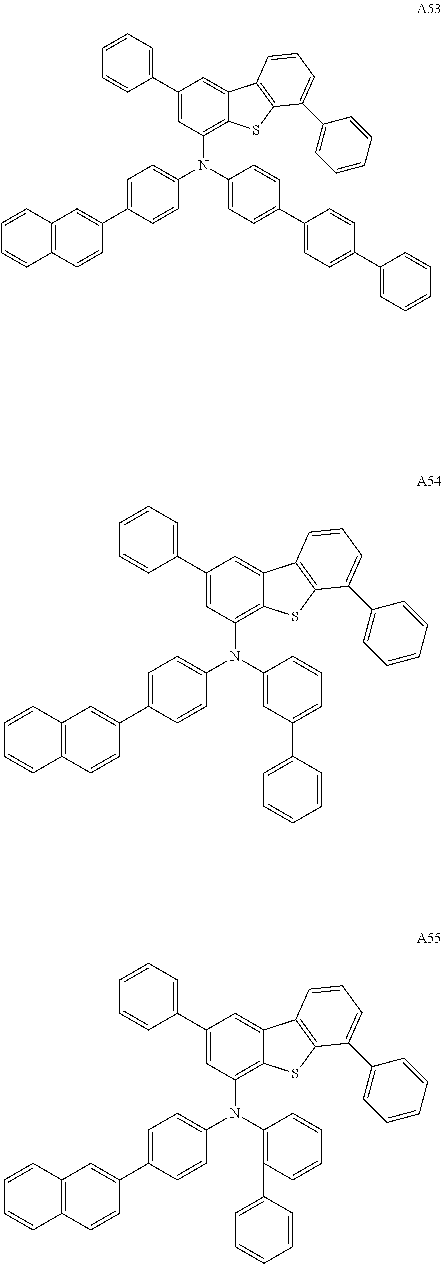 [SMILES: c1ccc(-c2ccc(-c3ccc(N(c4ccc(-c5ccc6ccccc6c5)cc4)c4cc(-c5ccccc5)cc5c4sc4c(-c6ccccc6)cccc45)cc3)cc2)cc1 ; c1ccc(-c2cccc(N(c3ccc(-c4ccc5ccccc5c4)cc3)c3cc(-c4ccccc4)cc4c3sc3c(-c5ccccc5)cccc34)c2)cc1 ; c1ccc(-c2cc(N(c3ccc(-c4ccc5ccccc5c4)cc3)c3ccccc3-c3ccccc3)c3sc4c(-c5ccccc5)cccc4c3c2)cc1]

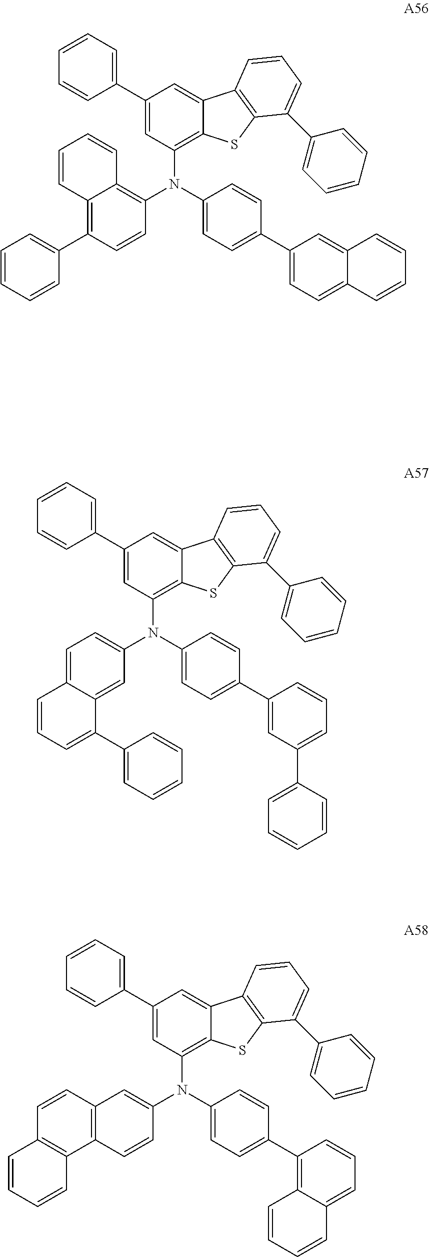 [SMILES: c1ccc(-c2cc(N(c3ccc(-c4ccc5ccccc5c4)cc3)c3ccc(-c4ccccc4)c4ccccc34)c3sc4c(-c5ccccc5)cccc4c3c2)cc1 ; c1ccc(-c2cccc(-c3ccc(N(c4ccc5cccc(-c6ccccc6)c5c4)c4cc(-c5ccccc5)cc5c4sc4c(-c6ccccc6)cccc45)cc3)c2)cc1 ; c1ccc(-c2cc(N(c3ccc(-c4cccc5ccccc45)cc3)c3ccc4c(ccc5ccccc54)c3)c3sc4c(-c5ccccc5)cccc4c3c2)cc1]

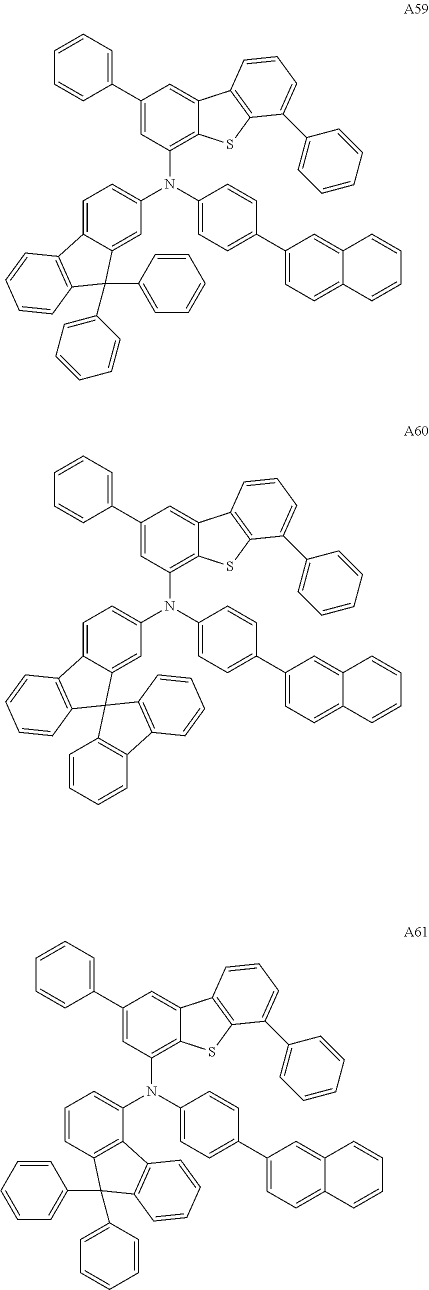 [SMILES: c1ccc(-c2cc(N(c3ccc(-c4ccc5ccccc5c4)cc3)c3ccc4c(c3)C(c3ccccc3)(c3ccccc3)c3ccccc3-4)c3sc4c(-c5ccccc5)cccc4c3c2)cc1 ; c1ccc(-c2cc(N(c3ccc(-c4ccc5ccccc5c4)cc3)c3ccc4c(c3)C3(c5ccccc5-c5ccccc53)c3ccccc3-4)c3sc4c(-c5ccccc5)cccc4c3c2)cc1 ; c1ccc(-c2cc(N(c3ccc(-c4ccc5ccccc5c4)cc3)c3cccc4c3-c3ccccc3C4(c3ccccc3)c3ccccc3)c3sc4c(-c5ccccc5)cccc4c3c2)cc1]

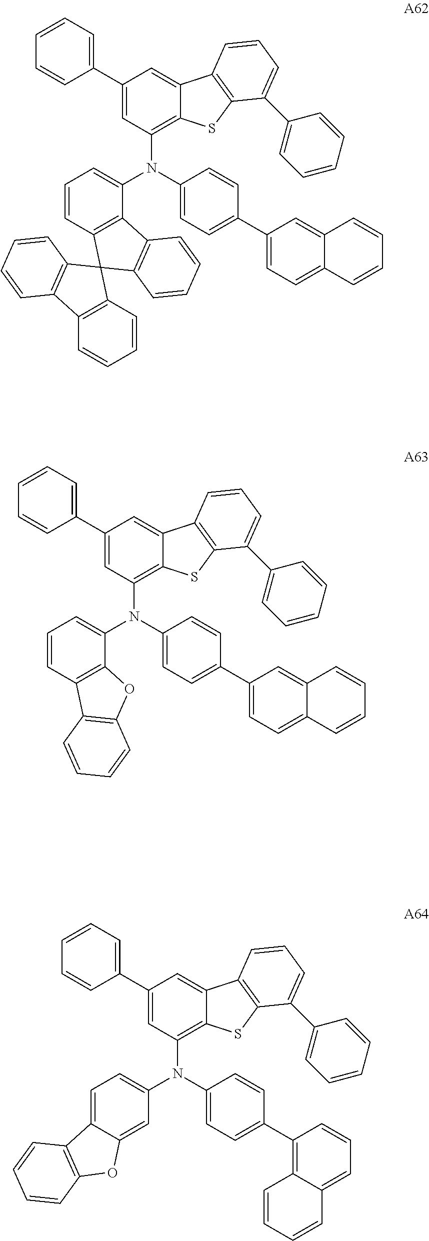 [SMILES: c1ccc(-c2cc(N(c3ccc(-c4ccc5ccccc5c4)cc3)c3cccc4c3-c3ccccc3C43c4ccccc4-c4ccccc43)c3sc4c(-c5ccccc5)cccc4c3c2)cc1 ; c1ccc(-c2cc(N(c3ccc(-c4ccc5ccccc5c4)cc3)c3cccc4c3oc3ccccc34)c3sc4c(-c5ccccc5)cccc4c3c2)cc1 ; c1ccc(-c2cc(N(c3ccc(-c4cccc5ccccc45)cc3)c3ccc4c(c3)oc3ccccc34)c3sc4c(-c5ccccc5)cccc4c3c2)cc1]

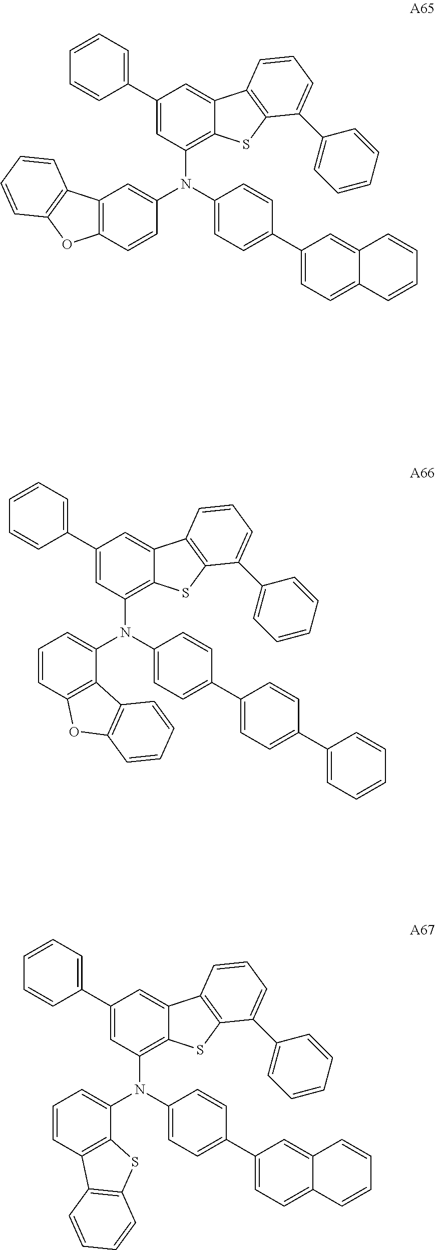 [SMILES: c1ccc(-c2cc(N(c3ccc(-c4ccc5ccccc5c4)cc3)c3ccc4oc5ccccc5c4c3)c3sc4c(-c5ccccc5)cccc4c3c2)cc1 ; c1ccc(-c2ccc(-c3ccc(N(c4cc(-c5ccccc5)cc5c4sc4c(-c6ccccc6)cccc45)c4cccc5oc6ccccc6c45)cc3)cc2)cc1 ; c1ccc(-c2cc(N(c3ccc(-c4ccc5ccccc5c4)cc3)c3cccc4c3sc3ccccc34)c3sc4c(-c5ccccc5)cccc4c3c2)cc1]

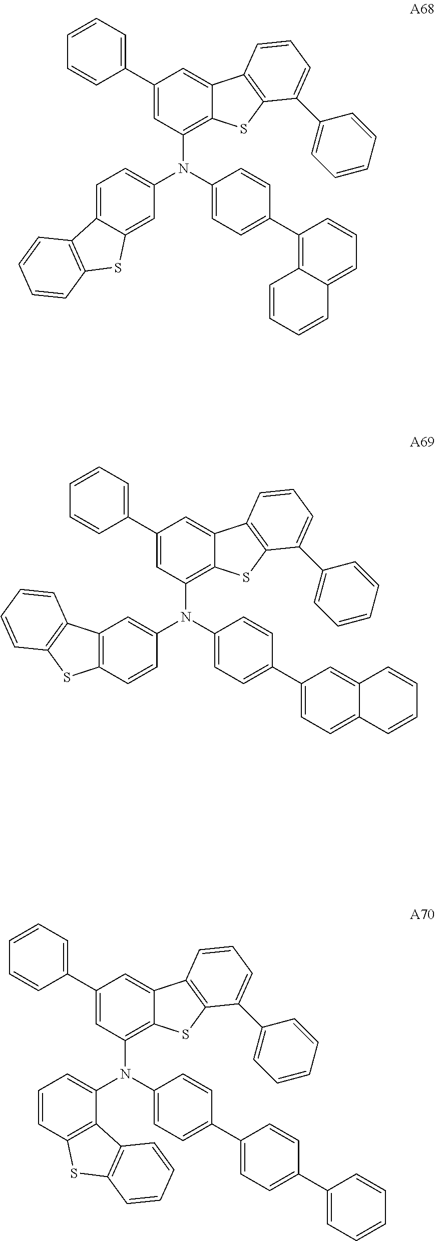 [SMILES: c1ccc(-c2cc(N(c3ccc(-c4cccc5ccccc45)cc3)c3ccc4c(c3)sc3ccccc34)c3sc4c(-c5ccccc5)cccc4c3c2)cc1 ; c1ccc(-c2cc(N(c3ccc(-c4ccc5ccccc5c4)cc3)c3ccc4sc5ccccc5c4c3)c3sc4c(-c5ccccc5)cccc4c3c2)cc1 ; c1ccc(-c2ccc(-c3ccc(N(c4cc(-c5ccccc5)cc5c4sc4c(-c6ccccc6)cccc45)c4cccc5sc6ccccc6c45)cc3)cc2)cc1]

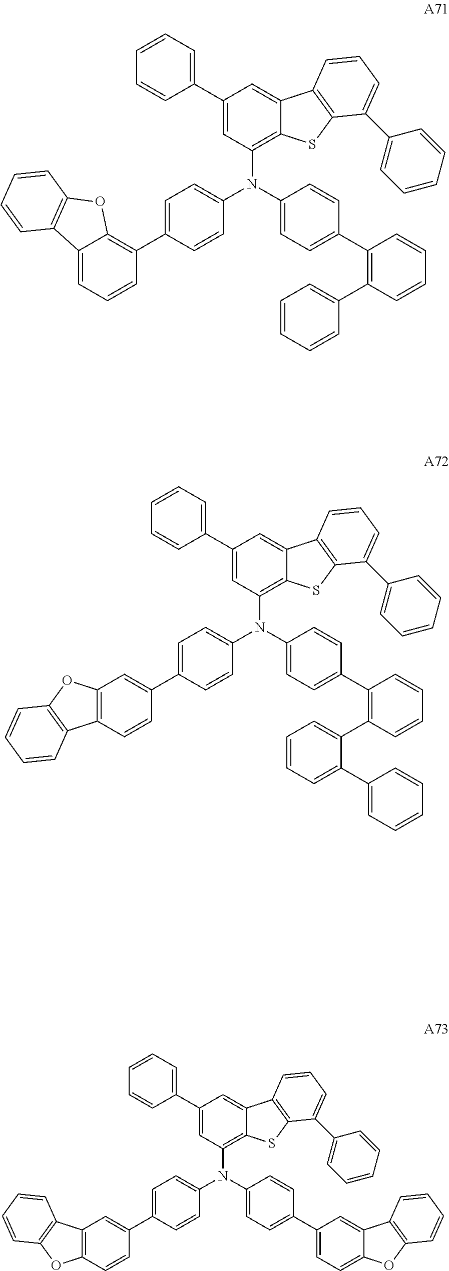 [SMILES: c1ccc(-c2cc(N(c3ccc(-c4ccccc4-c4ccccc4)cc3)c3ccc(-c4cccc5c4oc4ccccc45)cc3)c3sc4c(-c5ccccc5)cccc4c3c2)cc1 ; c1ccc(-c2cc(N(c3ccc(-c4ccc5c(c4)oc4ccccc45)cc3)c3ccc(-c4ccccc4-c4ccccc4-c4ccccc4)cc3)c3sc4c(-c5ccccc5)cccc4c3c2)cc1 ; c1ccc(-c2cc(N(c3ccc(-c4ccc5oc6ccccc6c5c4)cc3)c3ccc(-c4ccc5oc6ccccc6c5c4)cc3)c3sc4c(-c5ccccc5)cccc4c3c2)cc1]

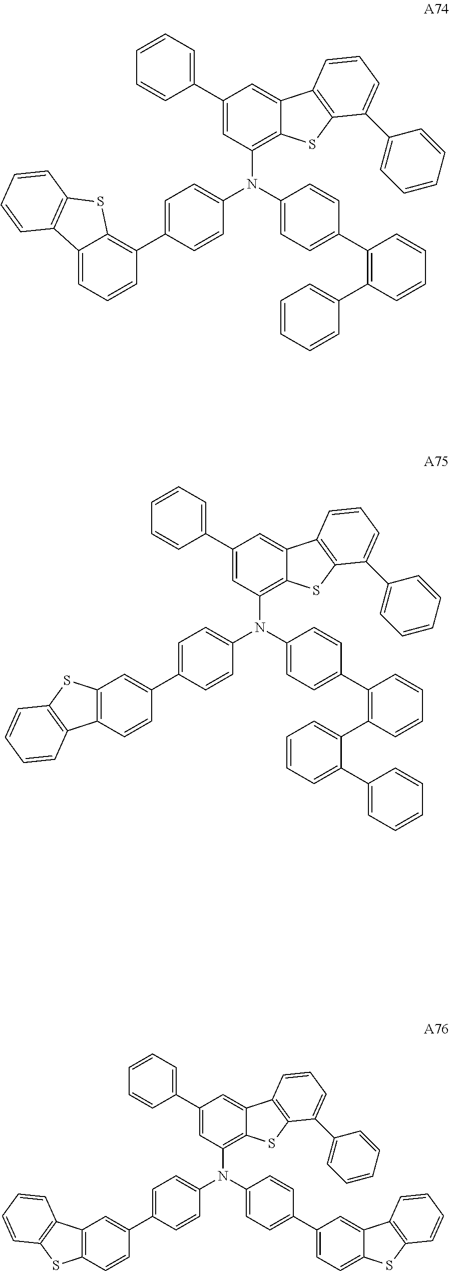 [SMILES: c1ccc(-c2cc(N(c3ccc(-c4ccccc4-c4ccccc4)cc3)c3ccc(-c4cccc5c4sc4ccccc45)cc3)c3sc4c(-c5ccccc5)cccc4c3c2)cc1 ; c1ccc(-c2cc(N(c3ccc(-c4ccc5c(c4)sc4ccccc45)cc3)c3ccc(-c4ccccc4-c4ccccc4-c4ccccc4)cc3)c3sc4c(-c5ccccc5)cccc4c3c2)cc1 ; c1ccc(-c2cc(N(c3ccc(-c4ccc5sc6ccccc6c5c4)cc3)c3ccc(-c4ccc5sc6ccccc6c5c4)cc3)c3sc4c(-c5ccccc5)cccc4c3c2)cc1]

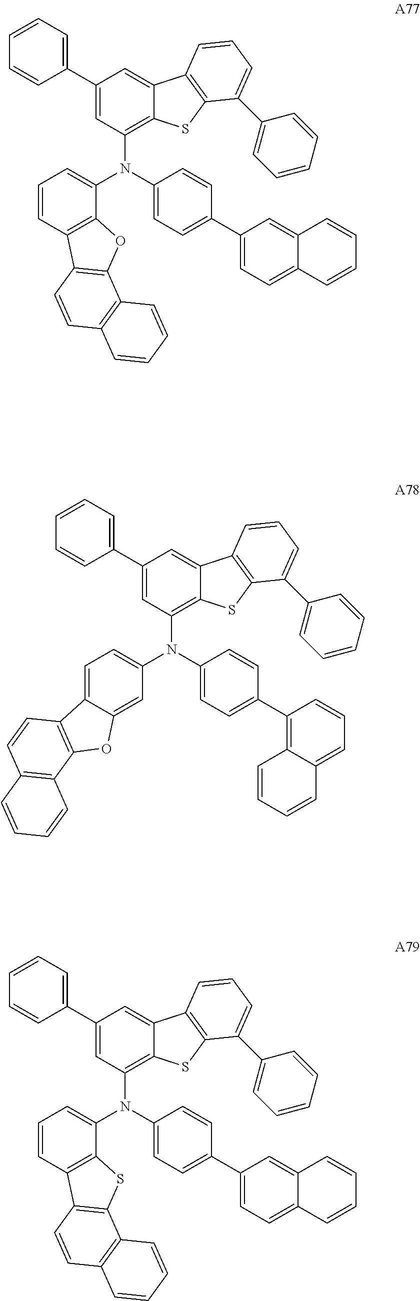 [SMILES: c1ccc(-c2cc(N(c3ccc(-c4ccc5ccccc5c4)cc3)c3cccc4c3oc3c5ccccc5ccc43)c3sc4c(-c5ccccc5)cccc4c3c2)cc1 ; c1ccc(-c2cc(N(c3ccc(-c4cccc5ccccc45)cc3)c3ccc4c(c3)oc3c5ccccc5ccc43)c3sc4c(-c5ccccc5)cccc4c3c2)cc1 ; c1ccc(-c2cc(N(c3ccc(-c4ccc5ccccc5c4)cc3)c3cccc4c3sc3c5ccccc5ccc43)c3sc4c(-c5ccccc5)cccc4c3c2)cc1]

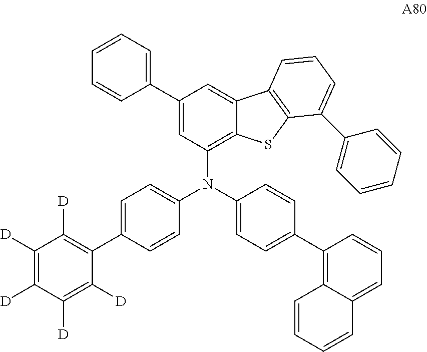 [SMILES: [2H]c1c([2H])c([2H])c(-c2ccc(N(c3ccc(-c4cccc5ccccc45)cc3)c3cc(-c4ccccc4)cc4c3sc3c(-c5ccccc5)cccc34)cc2)c([2H])c1[2H]]

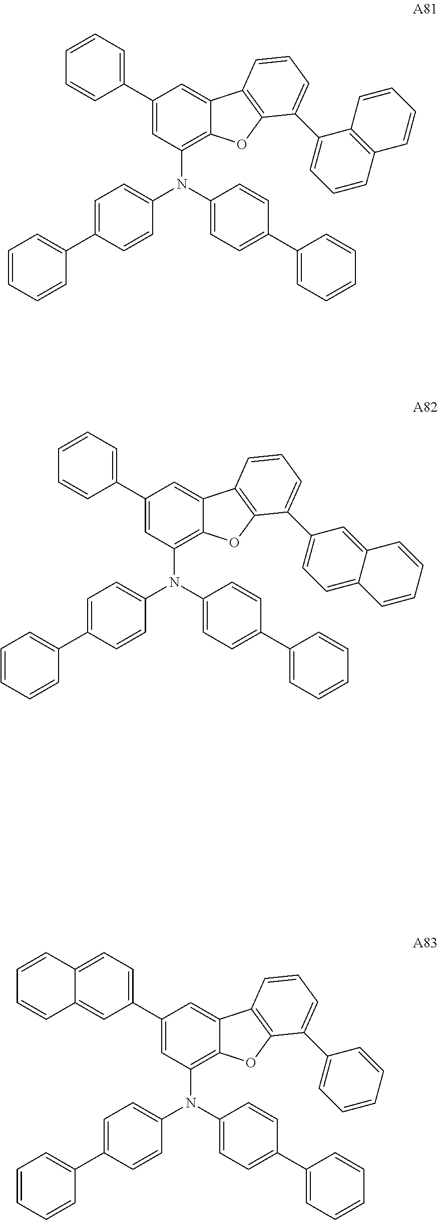 [SMILES: c1ccc(-c2ccc(N(c3ccc(-c4ccccc4)cc3)c3cc(-c4ccccc4)cc4c3oc3c(-c5cccc6ccccc56)cccc34)cc2)cc1 ; c1ccc(-c2ccc(N(c3ccc(-c4ccccc4)cc3)c3cc(-c4ccccc4)cc4c3oc3c(-c5ccc6ccccc6c5)cccc34)cc2)cc1 ; c1ccc(-c2ccc(N(c3ccc(-c4ccccc4)cc3)c3cc(-c4ccc5ccccc5c4)cc4c3oc3c(-c5ccccc5)cccc34)cc2)cc1]

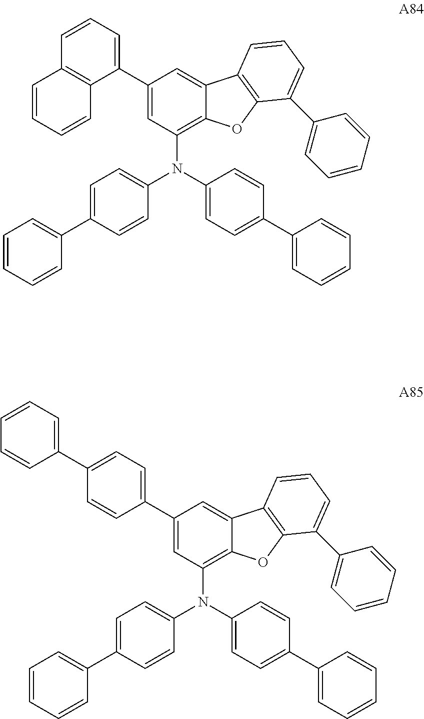 [SMILES: c1ccc(-c2ccc(N(c3ccc(-c4ccccc4)cc3)c3cc(-c4cccc5ccccc45)cc4c3oc3c(-c5ccccc5)cccc34)cc2)cc1 ; c1ccc(-c2ccc(-c3cc(N(c4ccc(-c5ccccc5)cc4)c4ccc(-c5ccccc5)cc4)c4oc5c(-c6ccccc6)cccc5c4c3)cc2)cc1]

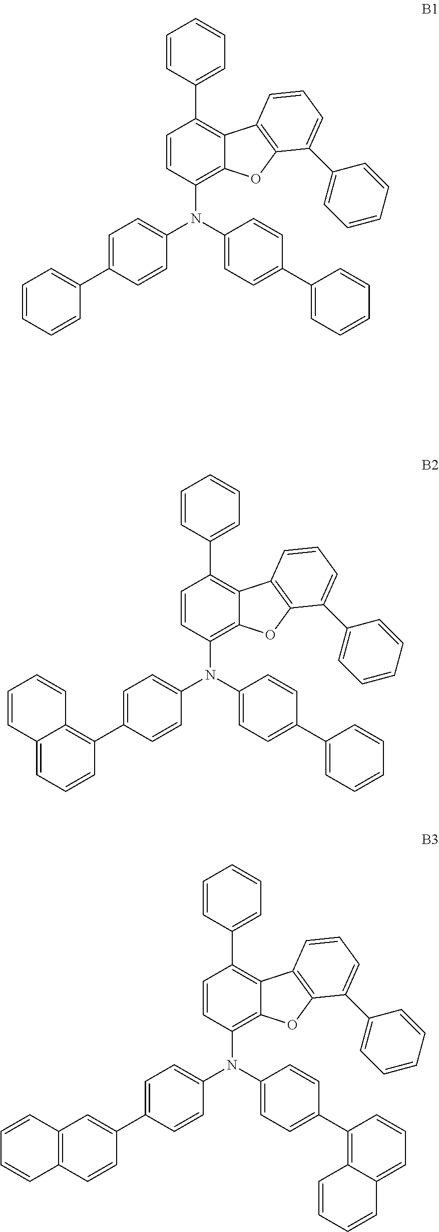 [SMILES: c1ccc(-c2ccc(N(c3ccc(-c4ccccc4)cc3)c3ccc(-c4ccccc4)c4c3oc3c(-c5ccccc5)cccc34)cc2)cc1 ; c1ccc(-c2ccc(N(c3ccc(-c4cccc5ccccc45)cc3)c3ccc(-c4ccccc4)c4c3oc3c(-c5ccccc5)cccc34)cc2)cc1 ; c1ccc(-c2cccc3c2oc2c(N(c4ccc(-c5ccc6ccccc6c5)cc4)c4ccc(-c5cccc6ccccc56)cc4)ccc(-c4ccccc4)c23)cc1]

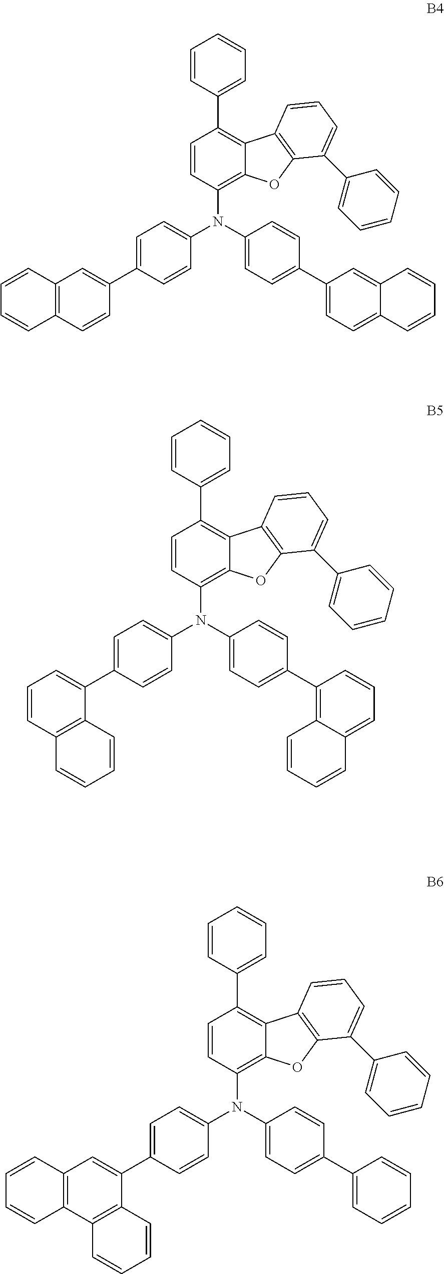 [SMILES: c1ccc(-c2cccc3c2oc2c(N(c4ccc(-c5ccc6ccccc6c5)cc4)c4ccc(-c5ccc6ccccc6c5)cc4)ccc(-c4ccccc4)c23)cc1 ; c1ccc(-c2cccc3c2oc2c(N(c4ccc(-c5cccc6ccccc56)cc4)c4ccc(-c5cccc6ccccc56)cc4)ccc(-c4ccccc4)c23)cc1 ; c1ccc(-c2ccc(N(c3ccc(-c4cc5ccccc5c5ccccc45)cc3)c3ccc(-c4ccccc4)c4c3oc3c(-c5ccccc5)cccc34)cc2)cc1]

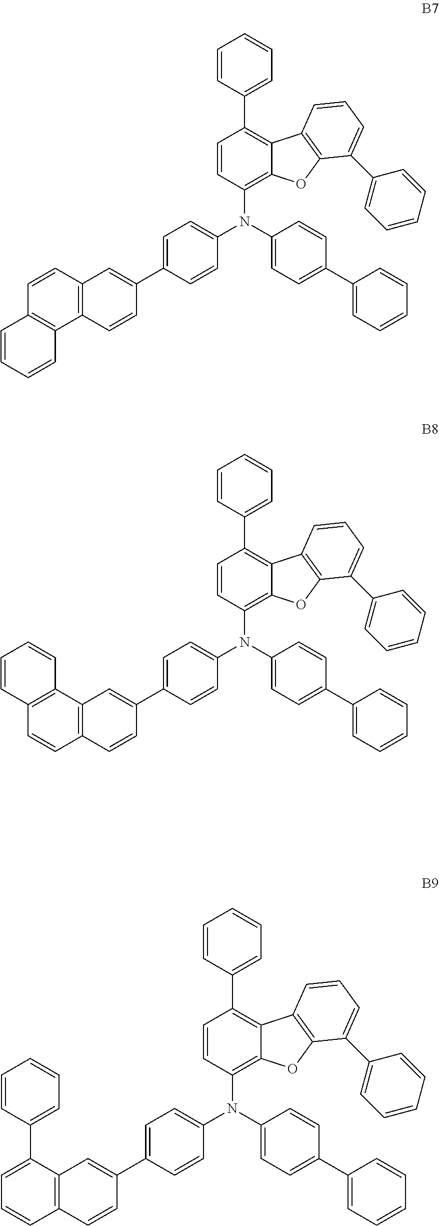 [SMILES: c1ccc(-c2ccc(N(c3ccc(-c4ccc5c(ccc6ccccc65)c4)cc3)c3ccc(-c4ccccc4)c4c3oc3c(-c5ccccc5)cccc34)cc2)cc1 ; c1ccc(-c2ccc(N(c3ccc(-c4ccc5ccc6ccccc6c5c4)cc3)c3ccc(-c4ccccc4)c4c3oc3c(-c5ccccc5)cccc34)cc2)cc1 ; c1ccc(-c2ccc(N(c3ccc(-c4ccc5cccc(-c6ccccc6)c5c4)cc3)c3ccc(-c4ccccc4)c4c3oc3c(-c5ccccc5)cccc34)cc2)cc1]

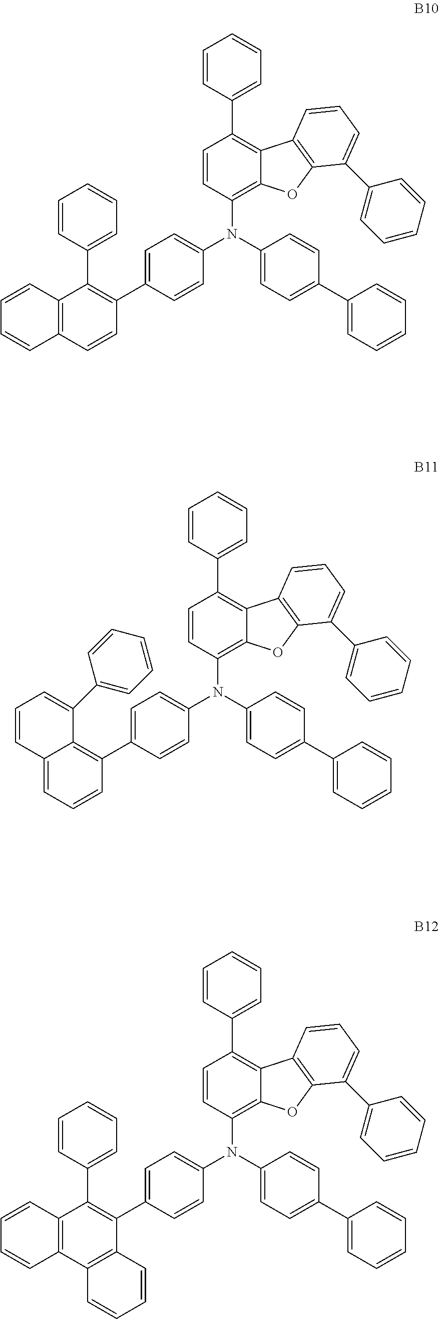 [SMILES: c1ccc(-c2ccc(N(c3ccc(-c4ccc5ccccc5c4-c4ccccc4)cc3)c3ccc(-c4ccccc4)c4c3oc3c(-c5ccccc5)cccc34)cc2)cc1 ; c1ccc(-c2ccc(N(c3ccc(-c4cccc5cccc(-c6ccccc6)c45)cc3)c3ccc(-c4ccccc4)c4c3oc3c(-c5ccccc5)cccc34)cc2)cc1 ; c1ccc(-c2ccc(N(c3ccc(-c4c(-c5ccccc5)c5ccccc5c5ccccc45)cc3)c3ccc(-c4ccccc4)c4c3oc3c(-c5ccccc5)cccc34)cc2)cc1]

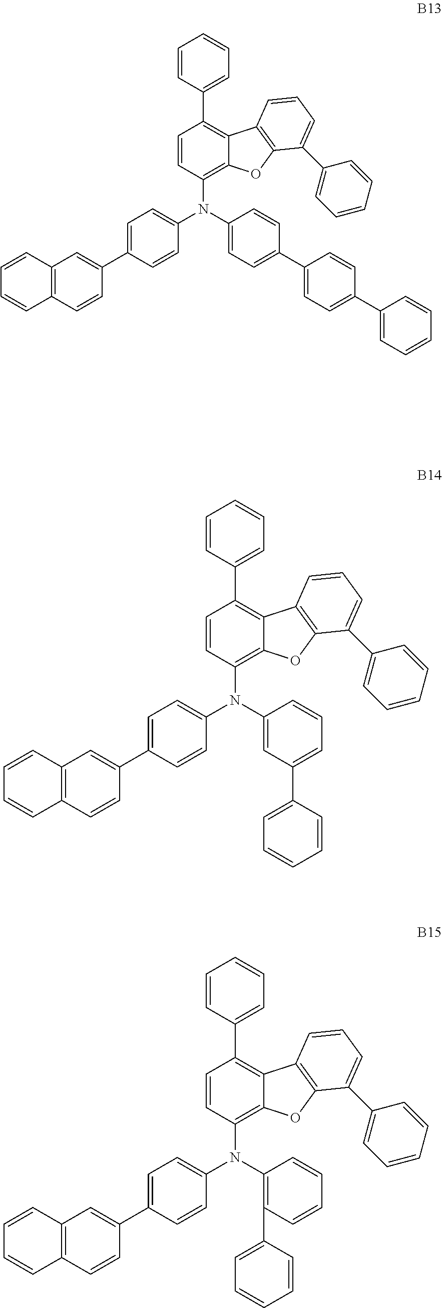 [SMILES: c1ccc(-c2ccc(-c3ccc(N(c4ccc(-c5ccc6ccccc6c5)cc4)c4ccc(-c5ccccc5)c5c4oc4c(-c6ccccc6)cccc45)cc3)cc2)cc1 ; c1ccc(-c2cccc(N(c3ccc(-c4ccc5ccccc5c4)cc3)c3ccc(-c4ccccc4)c4c3oc3c(-c5ccccc5)cccc34)c2)cc1 ; c1ccc(-c2ccccc2N(c2ccc(-c3ccc4ccccc4c3)cc2)c2ccc(-c3ccccc3)c3c2oc2c(-c4ccccc4)cccc23)cc1]

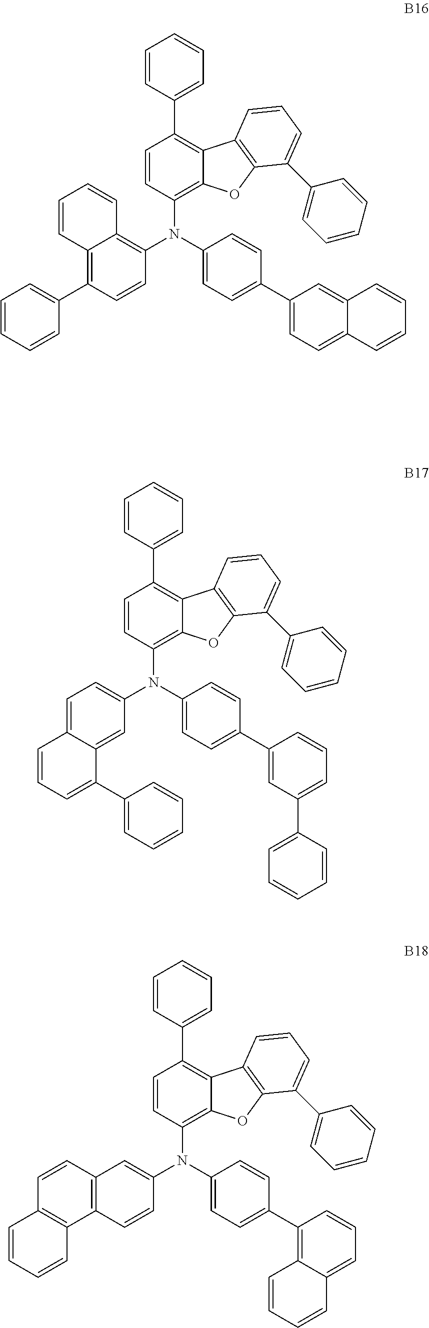 [SMILES: c1ccc(-c2ccc(N(c3ccc(-c4ccc5ccccc5c4)cc3)c3ccc(-c4ccccc4)c4c3oc3c(-c5ccccc5)cccc34)c3ccccc23)cc1 ; c1ccc(-c2cccc(-c3ccc(N(c4ccc5cccc(-c6ccccc6)c5c4)c4ccc(-c5ccccc5)c5c4oc4c(-c6ccccc6)cccc45)cc3)c2)cc1 ; c1ccc(-c2cccc3c2oc2c(N(c4ccc(-c5cccc6ccccc56)cc4)c4ccc5c(ccc6ccccc65)c4)ccc(-c4ccccc4)c23)cc1]

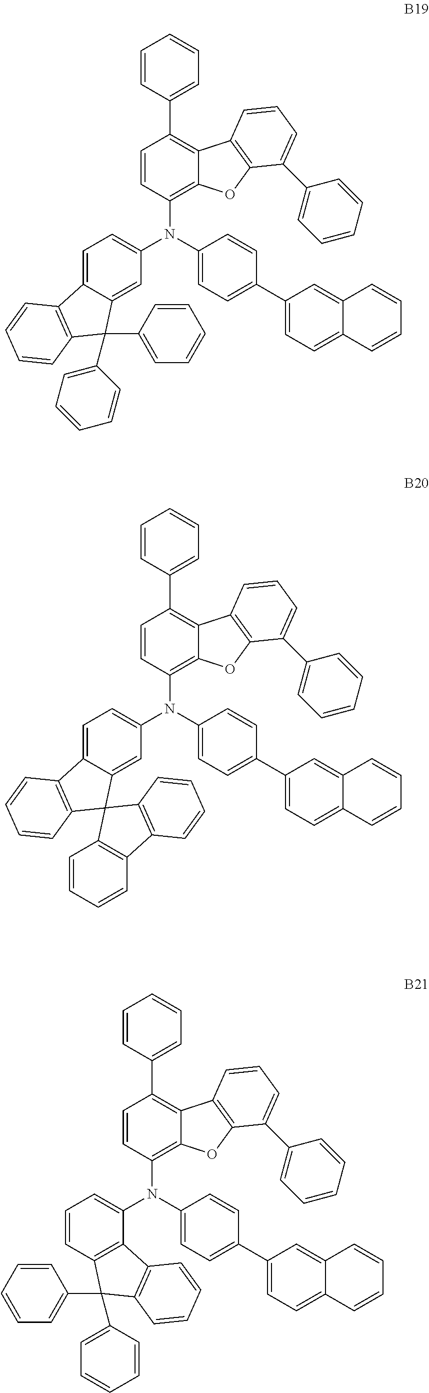 [SMILES: c1ccc(-c2cccc3c2oc2c(N(c4ccc(-c5ccc6ccccc6c5)cc4)c4ccc5c(c4)C(c4ccccc4)(c4ccccc4)c4ccccc4-5)ccc(-c4ccccc4)c23)cc1 ; c1ccc(-c2cccc3c2oc2c(N(c4ccc(-c5ccc6ccccc6c5)cc4)c4ccc5c(c4)C4(c6ccccc6-c6ccccc64)c4ccccc4-5)ccc(-c4ccccc4)c23)cc1 ; c1ccc(-c2cccc3c2oc2c(N(c4ccc(-c5ccc6ccccc6c5)cc4)c4cccc5c4-c4ccccc4C5(c4ccccc4)c4ccccc4)ccc(-c4ccccc4)c23)cc1]

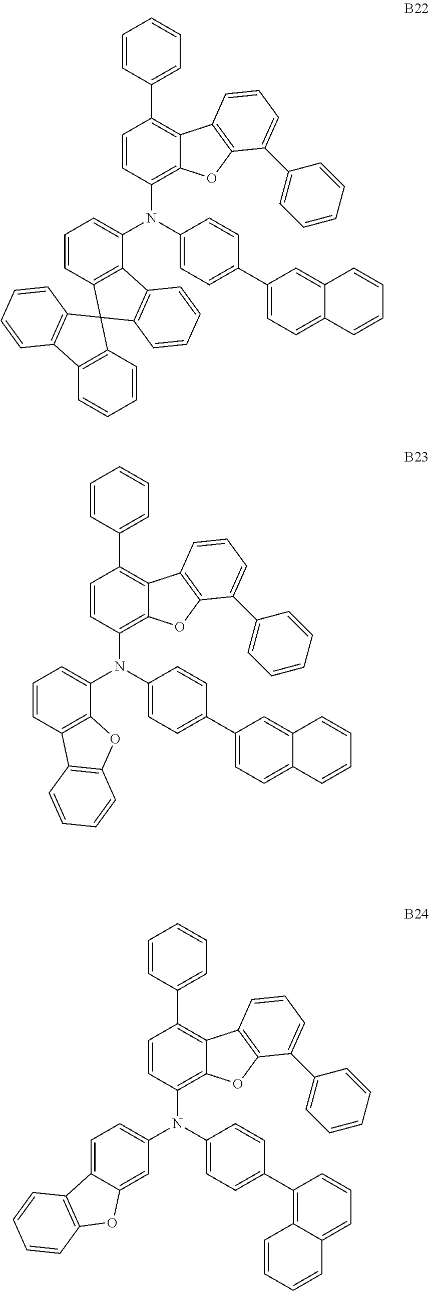 [SMILES: c1ccc(-c2cccc3c2oc2c(N(c4ccc(-c5ccc6ccccc6c5)cc4)c4cccc5c4-c4ccccc4C54c5ccccc5-c5ccccc54)ccc(-c4ccccc4)c23)cc1 ; c1ccc(-c2cccc3c2oc2c(N(c4ccc(-c5ccc6ccccc6c5)cc4)c4cccc5c4oc4ccccc45)ccc(-c4ccccc4)c23)cc1 ; c1ccc(-c2cccc3c2oc2c(N(c4ccc(-c5cccc6ccccc56)cc4)c4ccc5c(c4)oc4ccccc45)ccc(-c4ccccc4)c23)cc1]

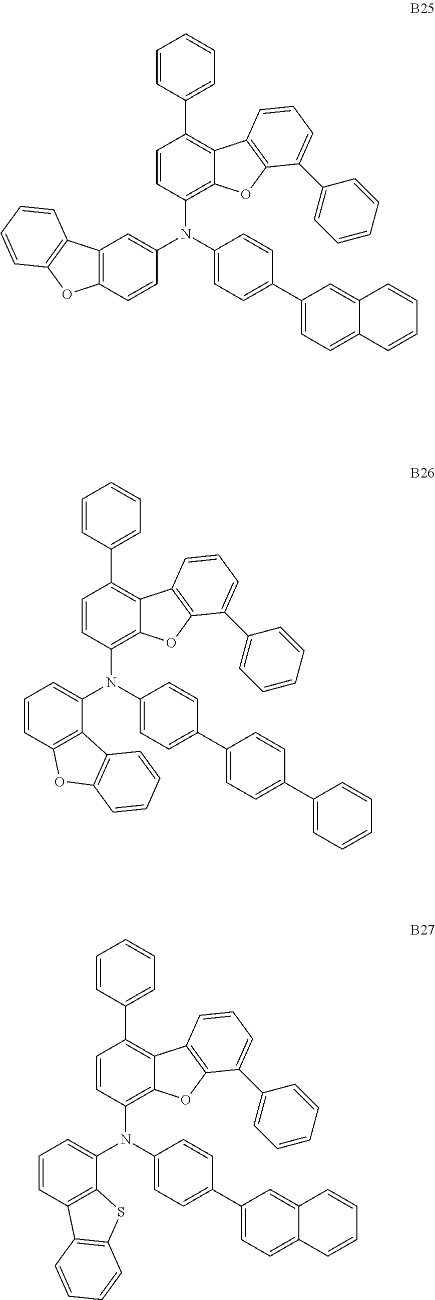 [SMILES: c1ccc(-c2cccc3c2oc2c(N(c4ccc(-c5ccc6ccccc6c5)cc4)c4ccc5oc6ccccc6c5c4)ccc(-c4ccccc4)c23)cc1 ; c1ccc(-c2ccc(-c3ccc(N(c4ccc(-c5ccccc5)c5c4oc4c(-c6ccccc6)cccc45)c4cccc5oc6ccccc6c45)cc3)cc2)cc1 ; c1ccc(-c2cccc3c2oc2c(N(c4ccc(-c5ccc6ccccc6c5)cc4)c4cccc5c4sc4ccccc45)ccc(-c4ccccc4)c23)cc1]

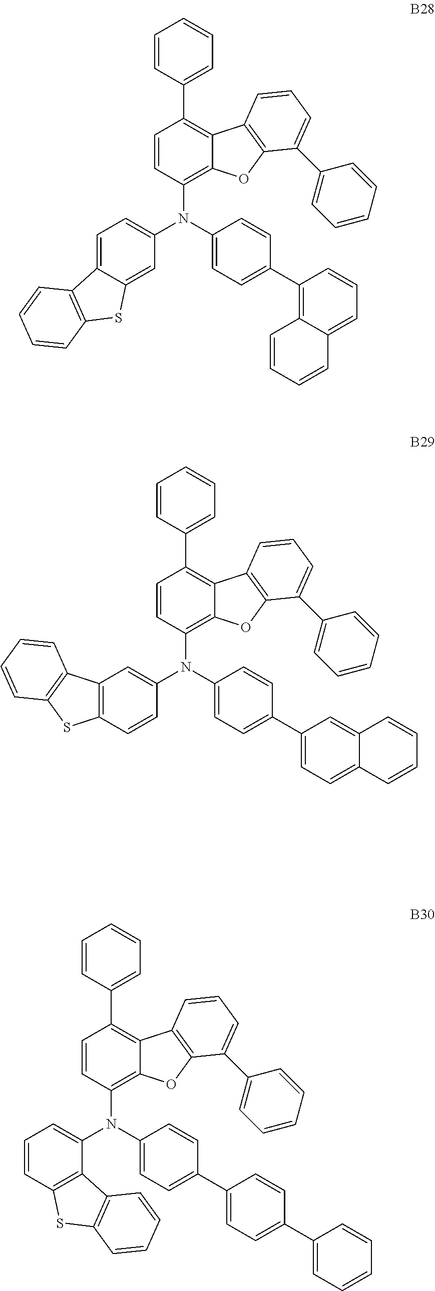 [SMILES: c1ccc(-c2cccc3c2oc2c(N(c4ccc(-c5cccc6ccccc56)cc4)c4ccc5c(c4)sc4ccccc45)ccc(-c4ccccc4)c23)cc1 ; c1ccc(-c2cccc3c2oc2c(N(c4ccc(-c5ccc6ccccc6c5)cc4)c4ccc5sc6ccccc6c5c4)ccc(-c4ccccc4)c23)cc1 ; c1ccc(-c2ccc(-c3ccc(N(c4ccc(-c5ccccc5)c5c4oc4c(-c6ccccc6)cccc45)c4cccc5sc6ccccc6c45)cc3)cc2)cc1]

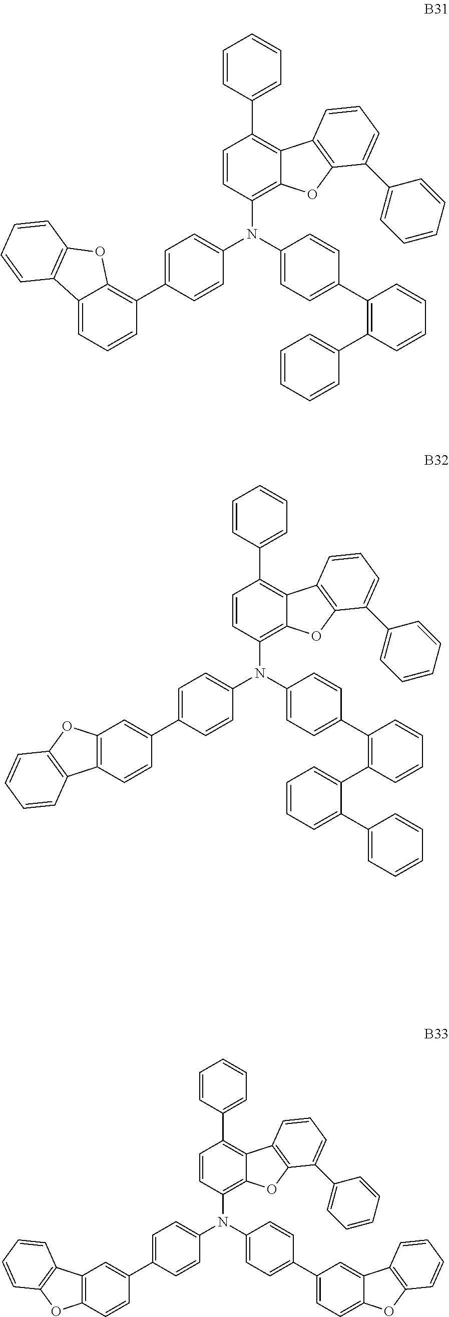 [SMILES: c1ccc(-c2ccccc2-c2ccc(N(c3ccc(-c4cccc5c4oc4ccccc45)cc3)c3ccc(-c4ccccc4)c4c3oc3c(-c5ccccc5)cccc34)cc2)cc1 ; c1ccc(-c2ccccc2-c2ccccc2-c2ccc(N(c3ccc(-c4ccc5c(c4)oc4ccccc45)cc3)c3ccc(-c4ccccc4)c4c3oc3c(-c5ccccc5)cccc34)cc2)cc1 ; c1ccc(-c2cccc3c2oc2c(N(c4ccc(-c5ccc6oc7ccccc7c6c5)cc4)c4ccc(-c5ccc6oc7ccccc7c6c5)cc4)ccc(-c4ccccc4)c23)cc1]

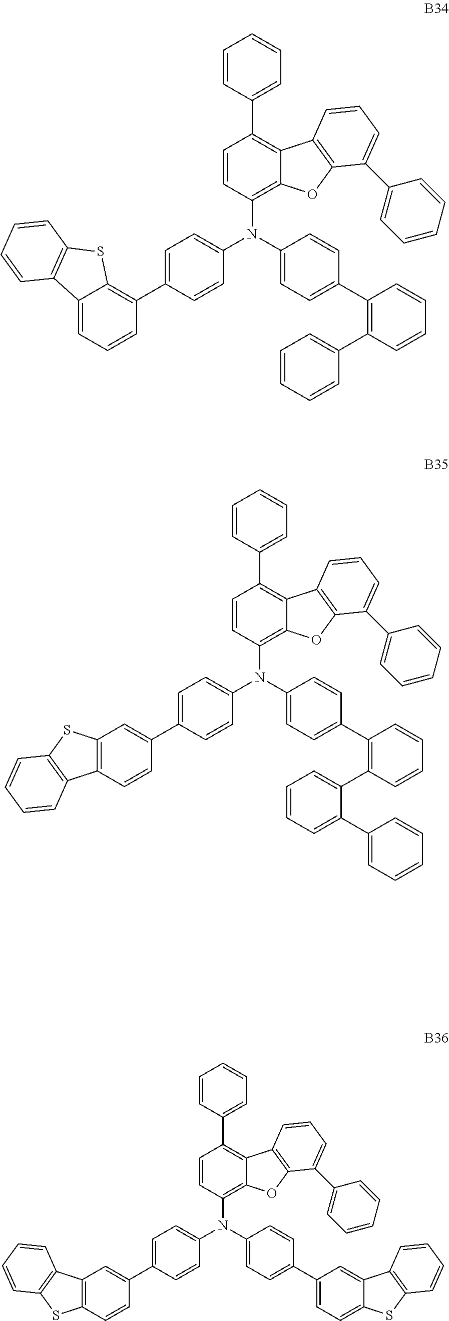 [SMILES: c1ccc(-c2ccccc2-c2ccc(N(c3ccc(-c4cccc5c4sc4ccccc45)cc3)c3ccc(-c4ccccc4)c4c3oc3c(-c5ccccc5)cccc34)cc2)cc1 ; c1ccc(-c2ccccc2-c2ccccc2-c2ccc(N(c3ccc(-c4ccc5c(c4)sc4ccccc45)cc3)c3ccc(-c4ccccc4)c4c3oc3c(-c5ccccc5)cccc34)cc2)cc1 ; c1ccc(-c2cccc3c2oc2c(N(c4ccc(-c5ccc6sc7ccccc7c6c5)cc4)c4ccc(-c5ccc6sc7ccccc7c6c5)cc4)ccc(-c4ccccc4)c23)cc1]

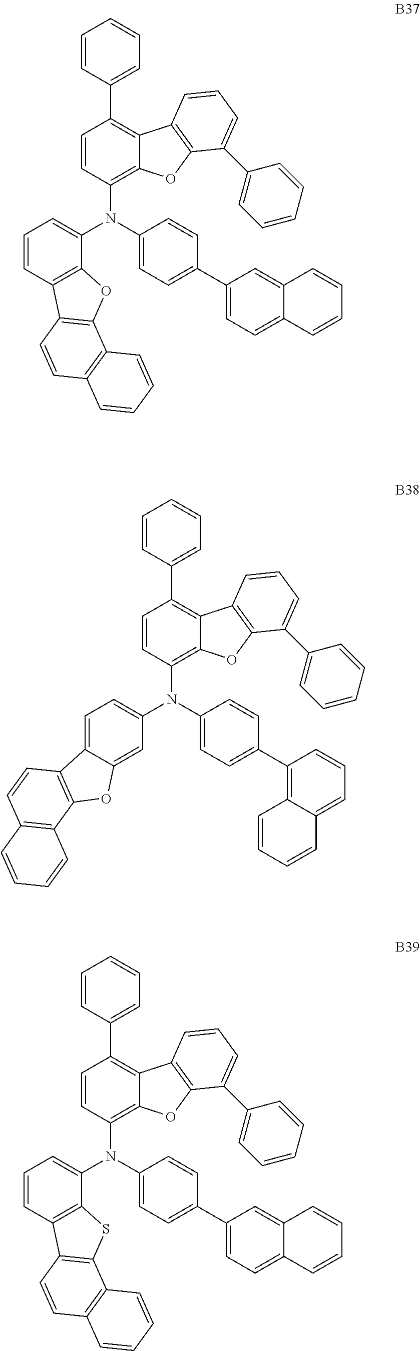 [SMILES: c1ccc(-c2cccc3c2oc2c(N(c4ccc(-c5ccc6ccccc6c5)cc4)c4cccc5c4oc4c6ccccc6ccc54)ccc(-c4ccccc4)c23)cc1 ; c1ccc(-c2cccc3c2oc2c(N(c4ccc(-c5cccc6ccccc56)cc4)c4ccc5c(c4)oc4c6ccccc6ccc54)ccc(-c4ccccc4)c23)cc1 ; c1ccc(-c2cccc3c2oc2c(N(c4ccc(-c5ccc6ccccc6c5)cc4)c4cccc5c4sc4c6ccccc6ccc54)ccc(-c4ccccc4)c23)cc1]

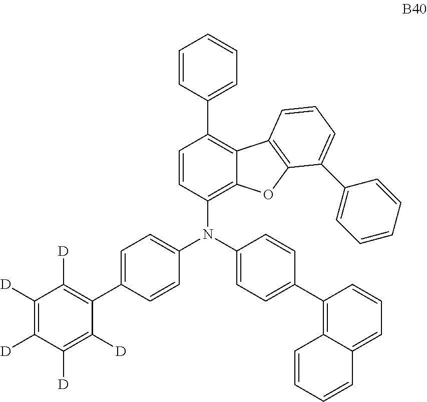 [SMILES: [2H]c1c([2H])c([2H])c(-c2ccc(N(c3ccc(-c4cccc5ccccc45)cc3)c3ccc(-c4ccccc4)c4c3oc3c(-c5ccccc5)cccc34)cc2)c([2H])c1[2H]]

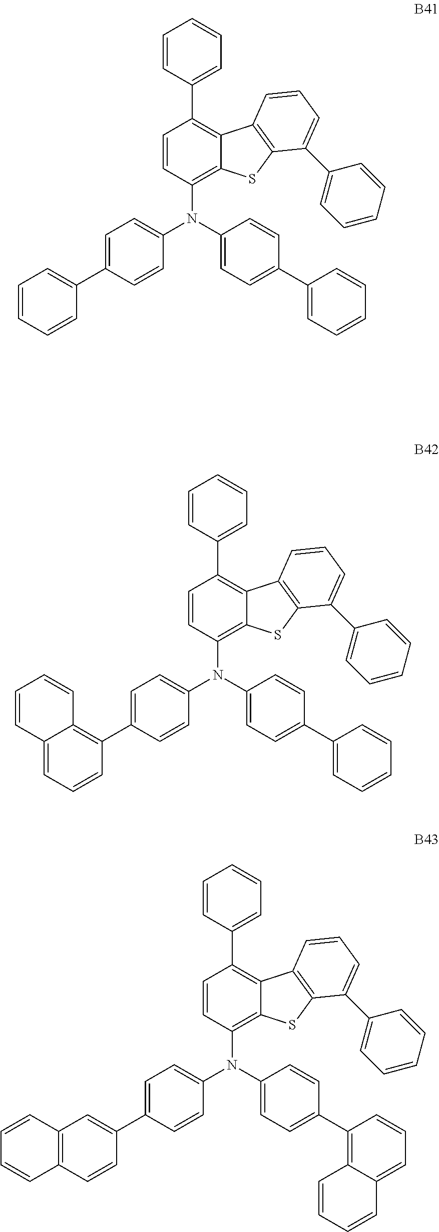 [SMILES: c1ccc(-c2ccc(N(c3ccc(-c4ccccc4)cc3)c3ccc(-c4ccccc4)c4c3sc3c(-c5ccccc5)cccc34)cc2)cc1 ; c1ccc(-c2ccc(N(c3ccc(-c4cccc5ccccc45)cc3)c3ccc(-c4ccccc4)c4c3sc3c(-c5ccccc5)cccc34)cc2)cc1 ; c1ccc(-c2cccc3c2sc2c(N(c4ccc(-c5ccc6ccccc6c5)cc4)c4ccc(-c5cccc6ccccc56)cc4)ccc(-c4ccccc4)c23)cc1]

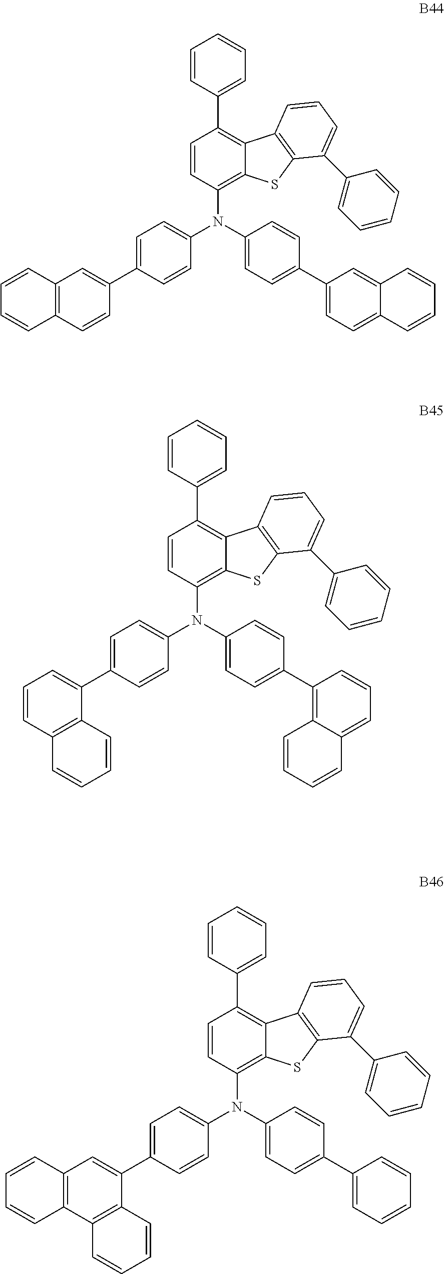 [SMILES: c1ccc(-c2cccc3c2sc2c(N(c4ccc(-c5ccc6ccccc6c5)cc4)c4ccc(-c5ccc6ccccc6c5)cc4)ccc(-c4ccccc4)c23)cc1 ; c1ccc(-c2cccc3c2sc2c(N(c4ccc(-c5cccc6ccccc56)cc4)c4ccc(-c5cccc6ccccc56)cc4)ccc(-c4ccccc4)c23)cc1 ; c1ccc(-c2ccc(N(c3ccc(-c4cc5ccccc5c5ccccc45)cc3)c3ccc(-c4ccccc4)c4c3sc3c(-c5ccccc5)cccc34)cc2)cc1]

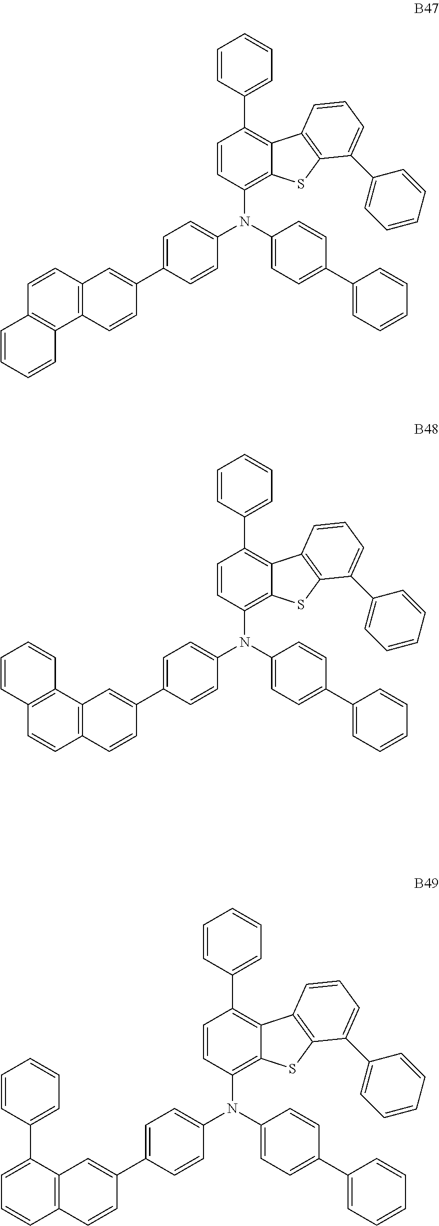 [SMILES: c1ccc(-c2ccc(N(c3ccc(-c4ccc5c(ccc6ccccc65)c4)cc3)c3ccc(-c4ccccc4)c4c3sc3c(-c5ccccc5)cccc34)cc2)cc1 ; c1ccc(-c2ccc(N(c3ccc(-c4ccc5ccc6ccccc6c5c4)cc3)c3ccc(-c4ccccc4)c4c3sc3c(-c5ccccc5)cccc34)cc2)cc1 ; c1ccc(-c2ccc(N(c3ccc(-c4ccc5cccc(-c6ccccc6)c5c4)cc3)c3ccc(-c4ccccc4)c4c3sc3c(-c5ccccc5)cccc34)cc2)cc1]

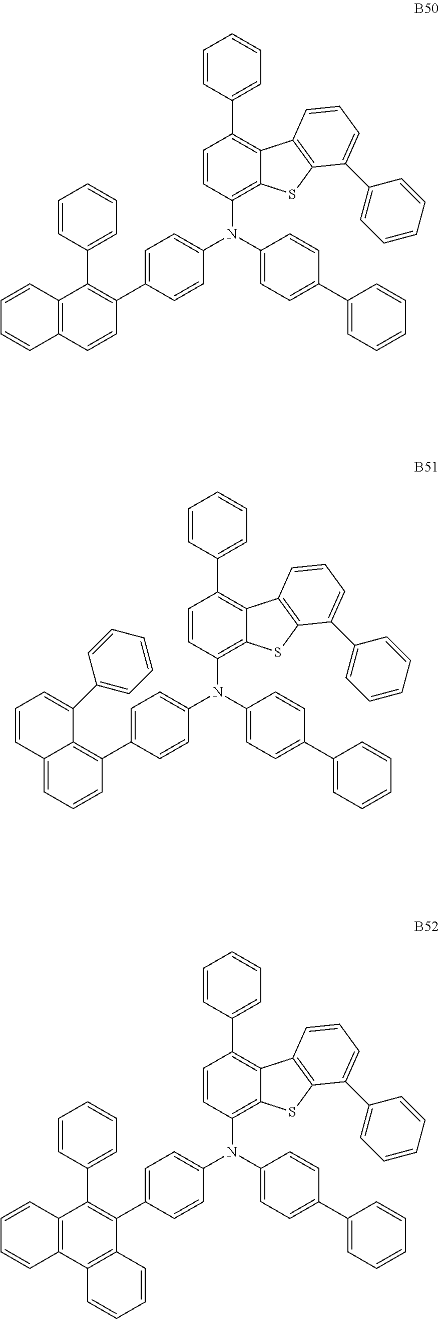 [SMILES: c1ccc(-c2ccc(N(c3ccc(-c4ccc5ccccc5c4-c4ccccc4)cc3)c3ccc(-c4ccccc4)c4c3sc3c(-c5ccccc5)cccc34)cc2)cc1 ; c1ccc(-c2ccc(N(c3ccc(-c4cccc5cccc(-c6ccccc6)c45)cc3)c3ccc(-c4ccccc4)c4c3sc3c(-c5ccccc5)cccc34)cc2)cc1 ; c1ccc(-c2ccc(N(c3ccc(-c4c(-c5ccccc5)c5ccccc5c5ccccc45)cc3)c3ccc(-c4ccccc4)c4c3sc3c(-c5ccccc5)cccc34)cc2)cc1]

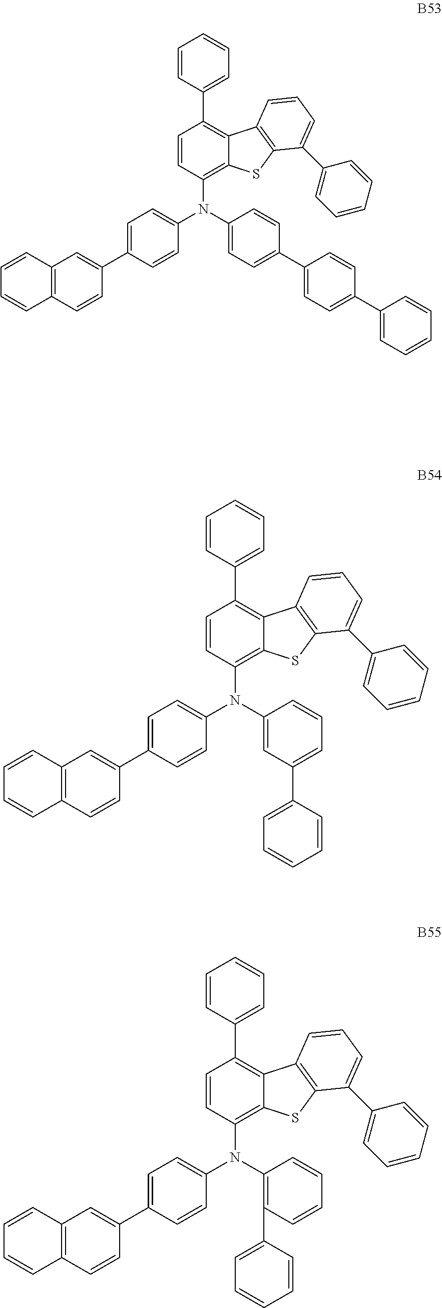 [SMILES: c1ccc(-c2ccc(-c3ccc(N(c4ccc(-c5ccc6ccccc6c5)cc4)c4ccc(-c5ccccc5)c5c4sc4c(-c6ccccc6)cccc45)cc3)cc2)cc1 ; c1ccc(-c2cccc(N(c3ccc(-c4ccc5ccccc5c4)cc3)c3ccc(-c4ccccc4)c4c3sc3c(-c5ccccc5)cccc34)c2)cc1 ; c1ccc(-c2ccccc2N(c2ccc(-c3ccc4ccccc4c3)cc2)c2ccc(-c3ccccc3)c3c2sc2c(-c4ccccc4)cccc23)cc1]

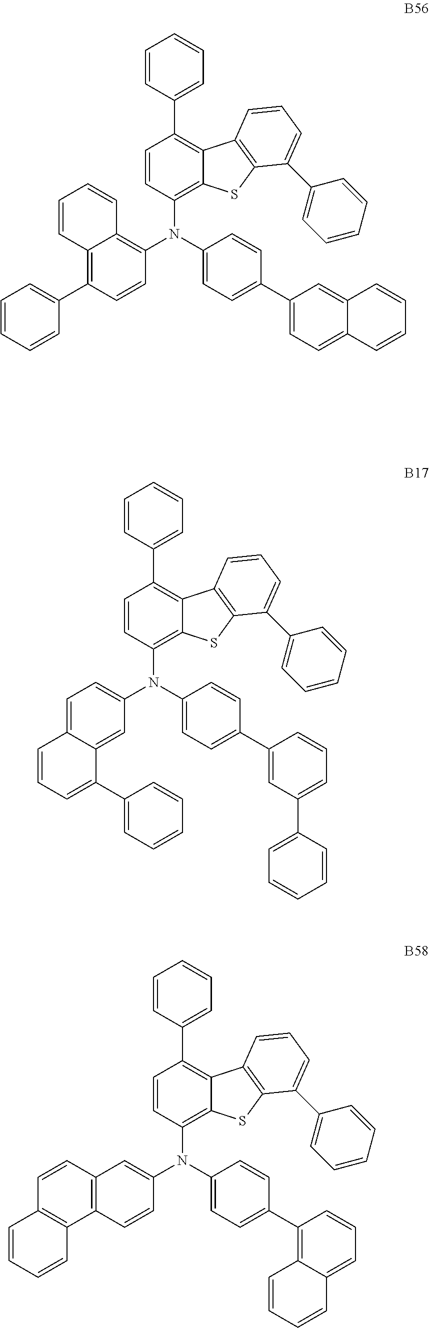 [SMILES: c1ccc(-c2ccc(N(c3ccc(-c4ccc5ccccc5c4)cc3)c3ccc(-c4ccccc4)c4c3sc3c(-c5ccccc5)cccc34)c3ccccc23)cc1 ; c1ccc(-c2cccc(-c3ccc(N(c4ccc5cccc(-c6ccccc6)c5c4)c4ccc(-c5ccccc5)c5c4sc4c(-c6ccccc6)cccc45)cc3)c2)cc1 ; c1ccc(-c2cccc3c2sc2c(N(c4ccc(-c5cccc6ccccc56)cc4)c4ccc5c(ccc6ccccc65)c4)ccc(-c4ccccc4)c23)cc1]

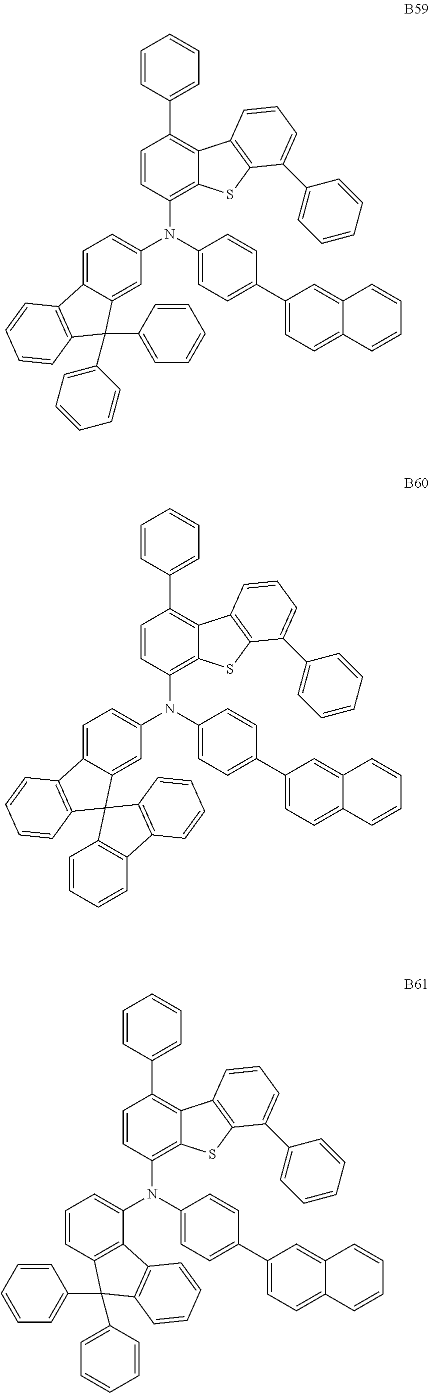 [SMILES: c1ccc(-c2cccc3c2sc2c(N(c4ccc(-c5ccc6ccccc6c5)cc4)c4ccc5c(c4)C(c4ccccc4)(c4ccccc4)c4ccccc4-5)ccc(-c4ccccc4)c23)cc1 ; c1ccc(-c2cccc3c2sc2c(N(c4ccc(-c5ccc6ccccc6c5)cc4)c4ccc5c(c4)C4(c6ccccc6-c6ccccc64)c4ccccc4-5)ccc(-c4ccccc4)c23)cc1 ; c1ccc(-c2cccc3c2sc2c(N(c4ccc(-c5ccc6ccccc6c5)cc4)c4cccc5c4-c4ccccc4C5(c4ccccc4)c4ccccc4)ccc(-c4ccccc4)c23)cc1]

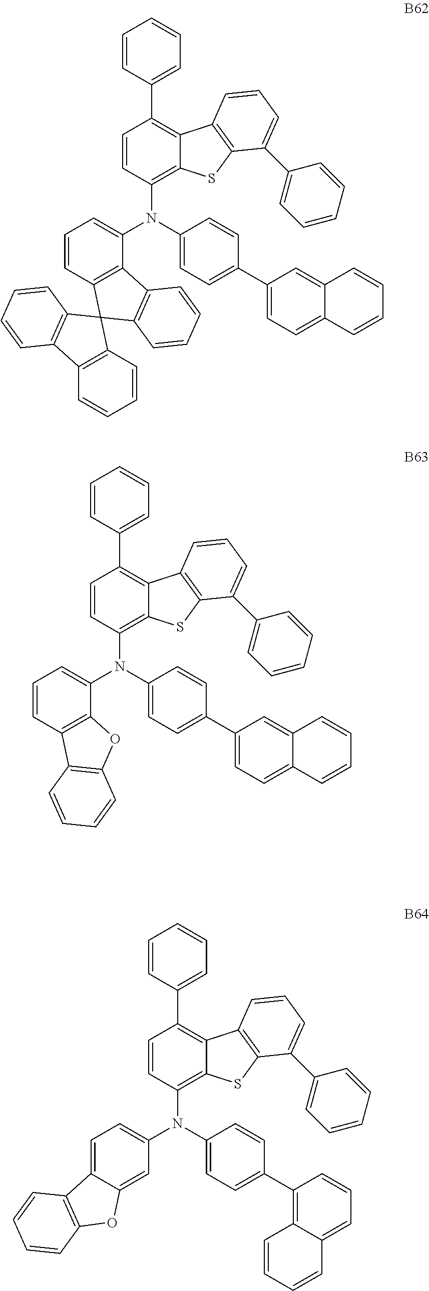 [SMILES: c1ccc(-c2cccc3c2sc2c(N(c4ccc(-c5ccc6ccccc6c5)cc4)c4cccc5c4-c4ccccc4C54c5ccccc5-c5ccccc54)ccc(-c4ccccc4)c23)cc1 ; c1ccc(-c2cccc3c2sc2c(N(c4ccc(-c5ccc6ccccc6c5)cc4)c4cccc5c4oc4ccccc45)ccc(-c4ccccc4)c23)cc1 ; c1ccc(-c2cccc3c2sc2c(N(c4ccc(-c5cccc6ccccc56)cc4)c4ccc5c(c4)oc4ccccc45)ccc(-c4ccccc4)c23)cc1]

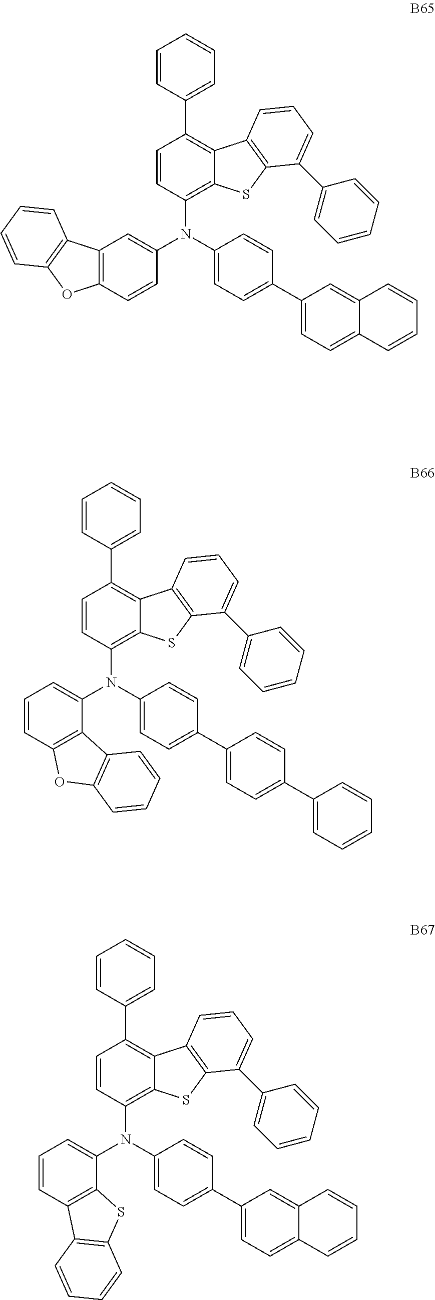 [SMILES: c1ccc(-c2cccc3c2sc2c(N(c4ccc(-c5ccc6ccccc6c5)cc4)c4ccc5oc6ccccc6c5c4)ccc(-c4ccccc4)c23)cc1 ; c1ccc(-c2ccc(-c3ccc(N(c4ccc(-c5ccccc5)c5c4sc4c(-c6ccccc6)cccc45)c4cccc5oc6ccccc6c45)cc3)cc2)cc1 ; c1ccc(-c2cccc3c2sc2c(N(c4ccc(-c5ccc6ccccc6c5)cc4)c4cccc5c4sc4ccccc45)ccc(-c4ccccc4)c23)cc1]

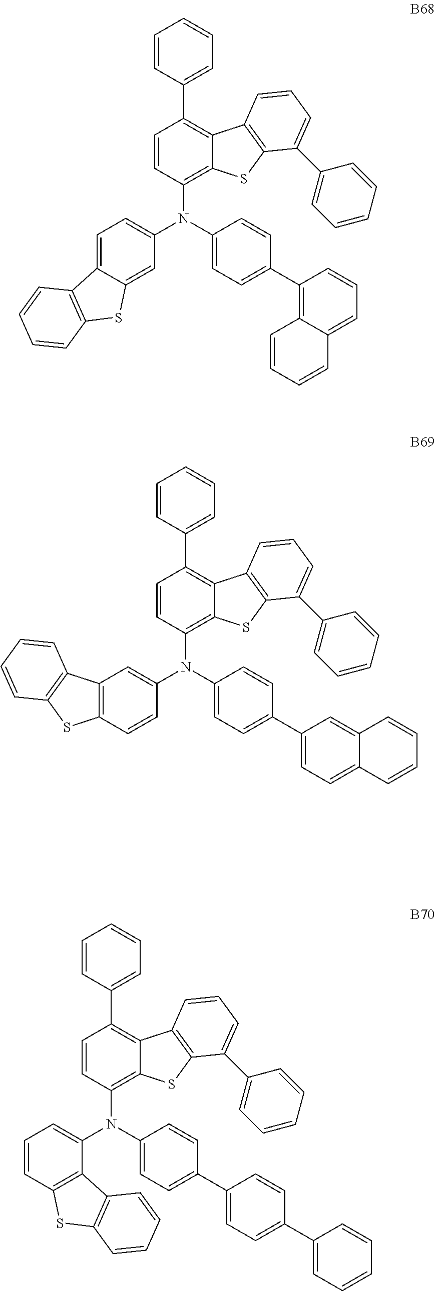 [SMILES: c1ccc(-c2cccc3c2sc2c(N(c4ccc(-c5cccc6ccccc56)cc4)c4ccc5c(c4)sc4ccccc45)ccc(-c4ccccc4)c23)cc1 ; c1ccc(-c2cccc3c2sc2c(N(c4ccc(-c5ccc6ccccc6c5)cc4)c4ccc5sc6ccccc6c5c4)ccc(-c4ccccc4)c23)cc1 ; c1ccc(-c2ccc(-c3ccc(N(c4ccc(-c5ccccc5)c5c4sc4c(-c6ccccc6)cccc45)c4cccc5sc6ccccc6c45)cc3)cc2)cc1]

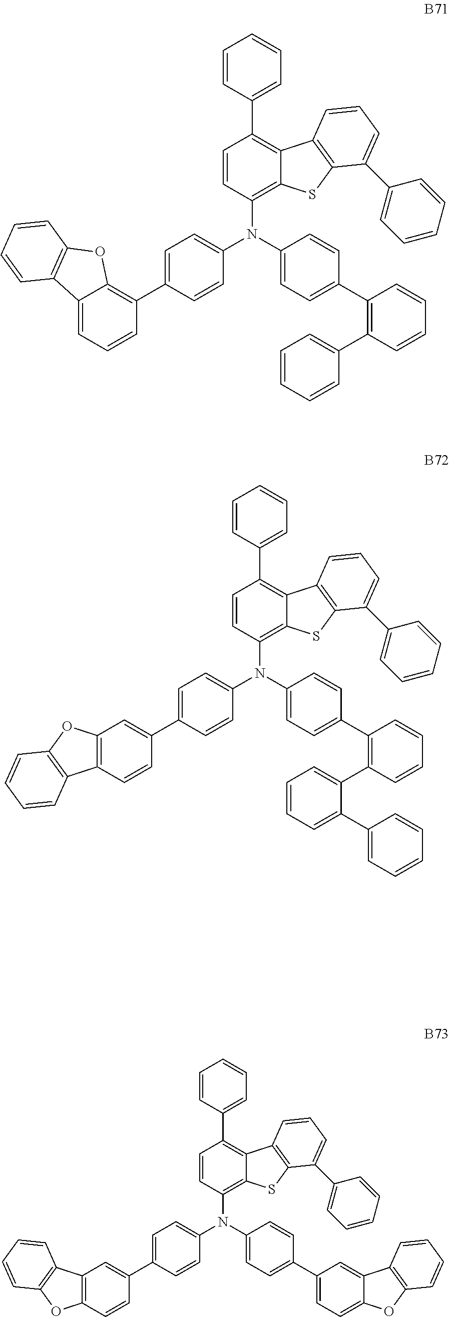 [SMILES: c1ccc(-c2ccccc2-c2ccc(N(c3ccc(-c4cccc5c4oc4ccccc45)cc3)c3ccc(-c4ccccc4)c4c3sc3c(-c5ccccc5)cccc34)cc2)cc1 ; c1ccc(-c2ccccc2-c2ccccc2-c2ccc(N(c3ccc(-c4ccc5c(c4)oc4ccccc45)cc3)c3ccc(-c4ccccc4)c4c3sc3c(-c5ccccc5)cccc34)cc2)cc1 ; c1ccc(-c2cccc3c2sc2c(N(c4ccc(-c5ccc6oc7ccccc7c6c5)cc4)c4ccc(-c5ccc6oc7ccccc7c6c5)cc4)ccc(-c4ccccc4)c23)cc1]

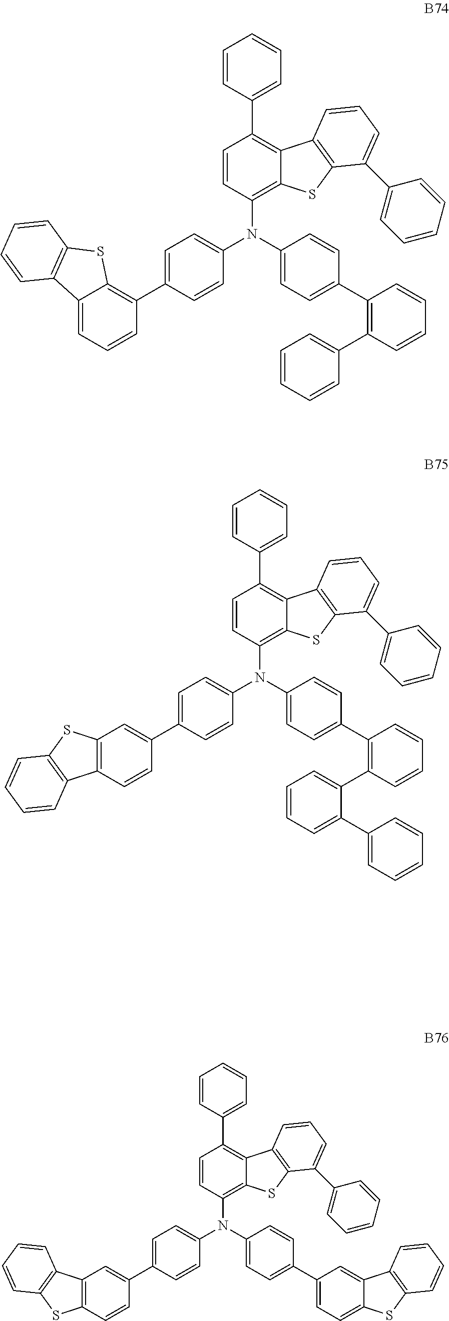 [SMILES: c1ccc(-c2ccccc2-c2ccc(N(c3ccc(-c4cccc5c4sc4ccccc45)cc3)c3ccc(-c4ccccc4)c4c3sc3c(-c5ccccc5)cccc34)cc2)cc1 ; c1ccc(-c2ccccc2-c2ccccc2-c2ccc(N(c3ccc(-c4ccc5c(c4)sc4ccccc45)cc3)c3ccc(-c4ccccc4)c4c3sc3c(-c5ccccc5)cccc34)cc2)cc1 ; c1ccc(-c2cccc3c2sc2c(N(c4ccc(-c5ccc6sc7ccccc7c6c5)cc4)c4ccc(-c5ccc6sc7ccccc7c6c5)cc4)ccc(-c4ccccc4)c23)cc1]

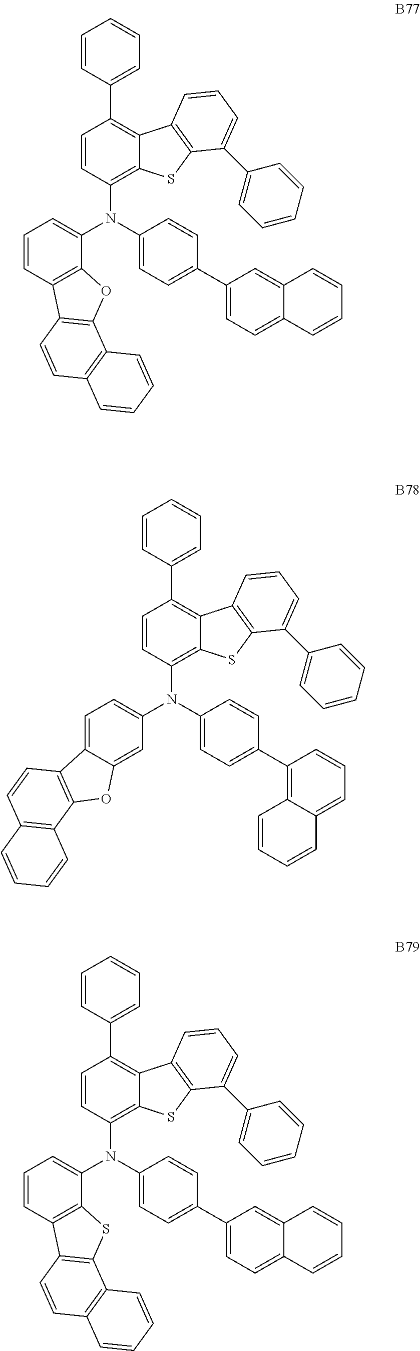 [SMILES: c1ccc(-c2cccc3c2sc2c(N(c4ccc(-c5ccc6ccccc6c5)cc4)c4cccc5c4oc4c6ccccc6ccc54)ccc(-c4ccccc4)c23)cc1 ; c1ccc(-c2cccc3c2sc2c(N(c4ccc(-c5cccc6ccccc56)cc4)c4ccc5c(c4)oc4c6ccccc6ccc54)ccc(-c4ccccc4)c23)cc1 ; c1ccc(-c2cccc3c2sc2c(N(c4ccc(-c5ccc6ccccc6c5)cc4)c4cccc5c4sc4c6ccccc6ccc54)ccc(-c4ccccc4)c23)cc1]

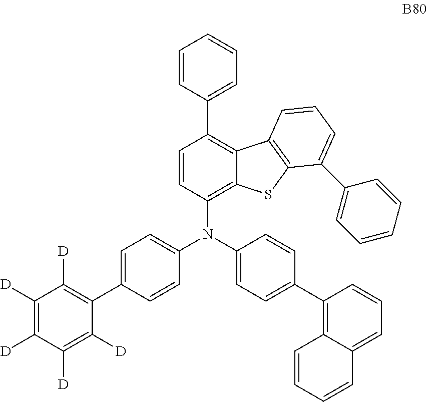 [SMILES: [2H]c1c([2H])c([2H])c(-c2ccc(N(c3ccc(-c4cccc5ccccc45)cc3)c3ccc(-c4ccccc4)c4c3sc3c(-c5ccccc5)cccc34)cc2)c([2H])c1[2H]]

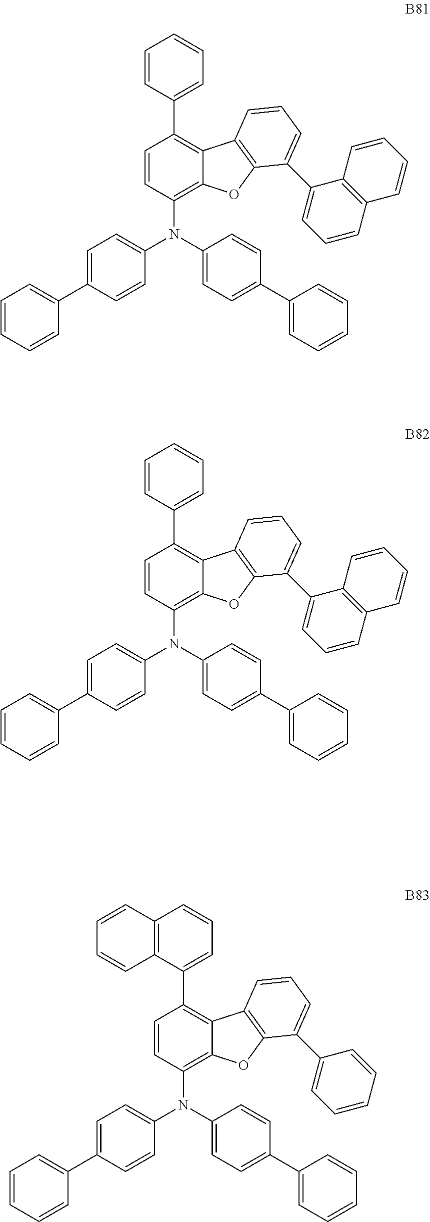 [SMILES: c1ccc(-c2ccc(N(c3ccc(-c4ccccc4)cc3)c3ccc(-c4ccccc4)c4c3oc3c(-c5cccc6ccccc56)cccc34)cc2)cc1 ; c1ccc(-c2ccc(N(c3ccc(-c4ccccc4)cc3)c3ccc(-c4ccccc4)c4c3oc3c(-c5cccc6ccccc56)cccc34)cc2)cc1 ; c1ccc(-c2ccc(N(c3ccc(-c4ccccc4)cc3)c3ccc(-c4cccc5ccccc45)c4c3oc3c(-c5ccccc5)cccc34)cc2)cc1]

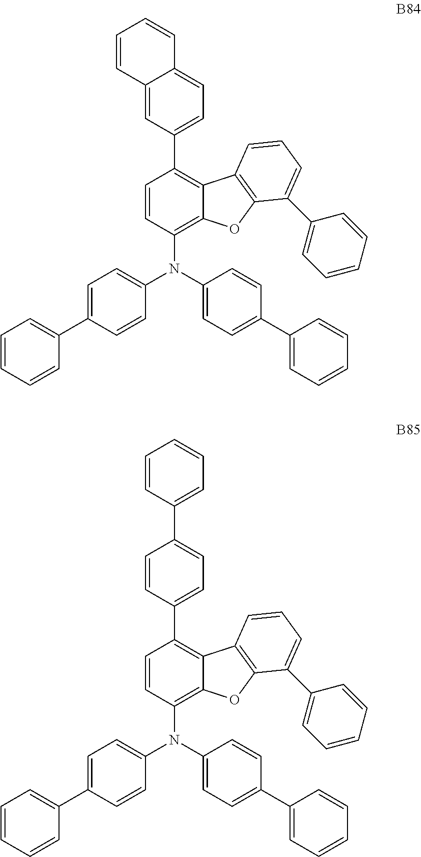 [SMILES: c1ccc(-c2ccc(N(c3ccc(-c4ccccc4)cc3)c3ccc(-c4ccc5ccccc5c4)c4c3oc3c(-c5ccccc5)cccc34)cc2)cc1 ; c1ccc(-c2ccc(-c3ccc(N(c4ccc(-c5ccccc5)cc4)c4ccc(-c5ccccc5)cc4)c4oc5c(-c6ccccc6)cccc5c34)cc2)cc1]

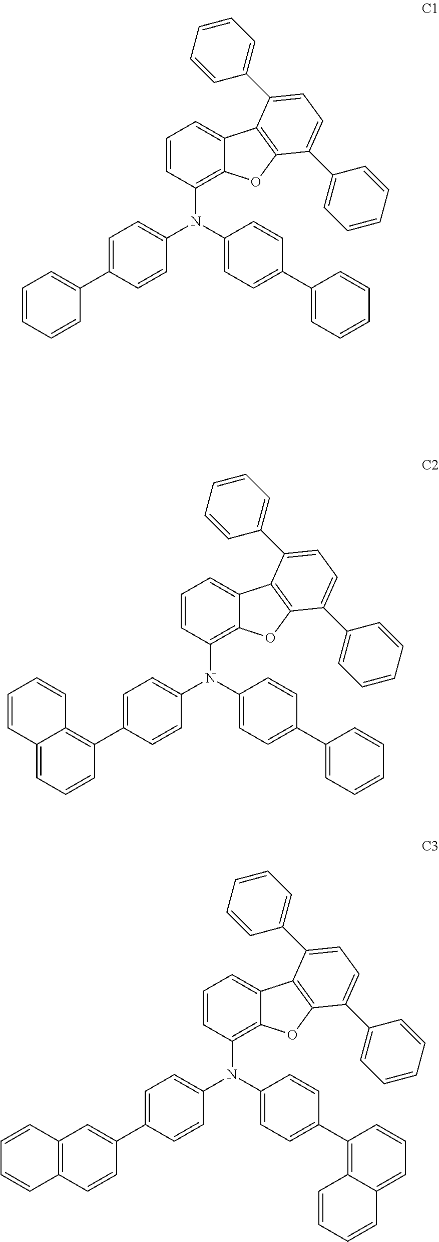 [SMILES: c1ccc(-c2ccc(N(c3ccc(-c4ccccc4)cc3)c3cccc4c3oc3c(-c5ccccc5)ccc(-c5ccccc5)c34)cc2)cc1 ; c1ccc(-c2ccc(N(c3ccc(-c4cccc5ccccc45)cc3)c3cccc4c3oc3c(-c5ccccc5)ccc(-c5ccccc5)c34)cc2)cc1 ; c1ccc(-c2ccc(-c3ccccc3)c3c2oc2c(N(c4ccc(-c5ccc6ccccc6c5)cc4)c4ccc(-c5cccc6ccccc56)cc4)cccc23)cc1]

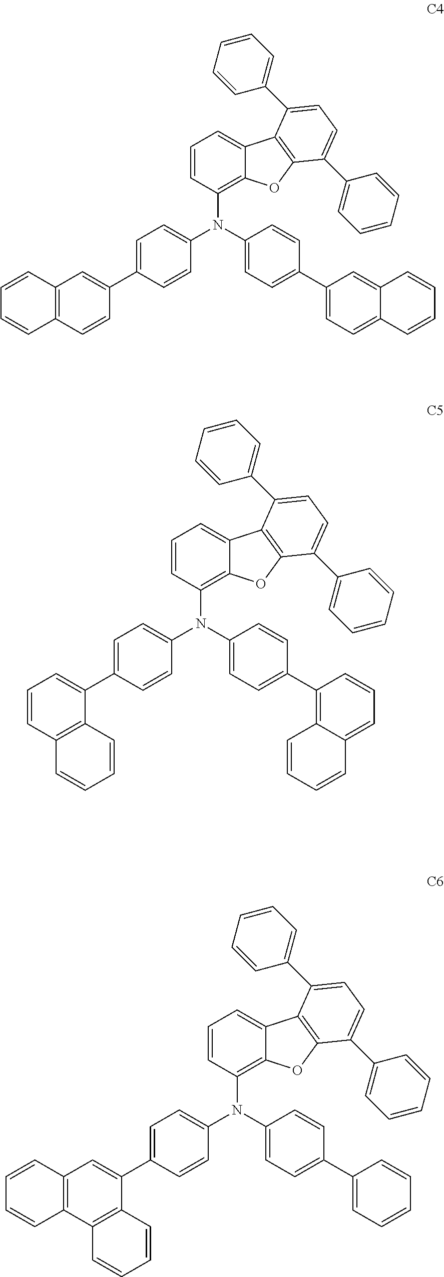 [SMILES: c1ccc(-c2ccc(-c3ccccc3)c3c2oc2c(N(c4ccc(-c5ccc6ccccc6c5)cc4)c4ccc(-c5ccc6ccccc6c5)cc4)cccc23)cc1 ; c1ccc(-c2ccc(-c3ccccc3)c3c2oc2c(N(c4ccc(-c5cccc6ccccc56)cc4)c4ccc(-c5cccc6ccccc56)cc4)cccc23)cc1 ; c1ccc(-c2ccc(N(c3ccc(-c4cc5ccccc5c5ccccc45)cc3)c3cccc4c3oc3c(-c5ccccc5)ccc(-c5ccccc5)c34)cc2)cc1]

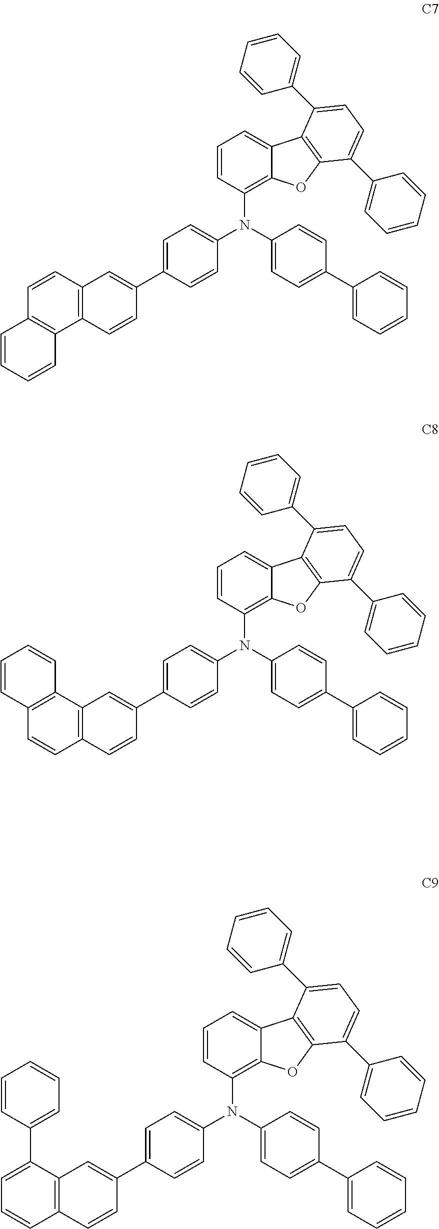 [SMILES: c1ccc(-c2ccc(N(c3ccc(-c4ccc5c(ccc6ccccc65)c4)cc3)c3cccc4c3oc3c(-c5ccccc5)ccc(-c5ccccc5)c34)cc2)cc1 ; c1ccc(-c2ccc(N(c3ccc(-c4ccc5ccc6ccccc6c5c4)cc3)c3cccc4c3oc3c(-c5ccccc5)ccc(-c5ccccc5)c34)cc2)cc1 ; c1ccc(-c2ccc(N(c3ccc(-c4ccc5cccc(-c6ccccc6)c5c4)cc3)c3cccc4c3oc3c(-c5ccccc5)ccc(-c5ccccc5)c34)cc2)cc1]

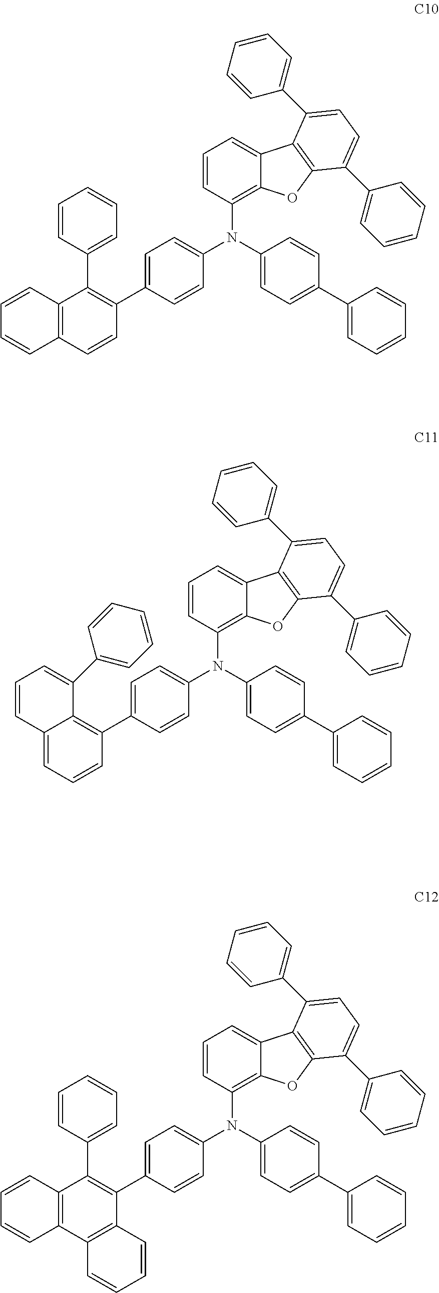 [SMILES: c1ccc(-c2ccc(N(c3ccc(-c4ccc5ccccc5c4-c4ccccc4)cc3)c3cccc4c3oc3c(-c5ccccc5)ccc(-c5ccccc5)c34)cc2)cc1 ; c1ccc(-c2ccc(N(c3ccc(-c4cccc5cccc(-c6ccccc6)c45)cc3)c3cccc4c3oc3c(-c5ccccc5)ccc(-c5ccccc5)c34)cc2)cc1 ; c1ccc(-c2ccc(N(c3ccc(-c4c(-c5ccccc5)c5ccccc5c5ccccc45)cc3)c3cccc4c3oc3c(-c5ccccc5)ccc(-c5ccccc5)c34)cc2)cc1]

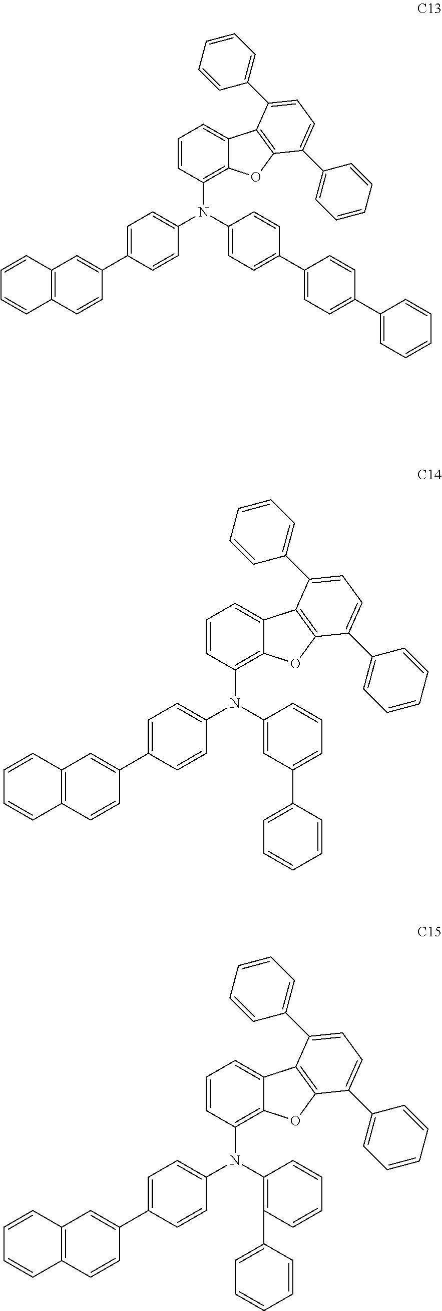 [SMILES: c1ccc(-c2ccc(-c3ccc(N(c4ccc(-c5ccc6ccccc6c5)cc4)c4cccc5c4oc4c(-c6ccccc6)ccc(-c6ccccc6)c45)cc3)cc2)cc1 ; c1ccc(-c2cccc(N(c3ccc(-c4ccc5ccccc5c4)cc3)c3cccc4c3oc3c(-c5ccccc5)ccc(-c5ccccc5)c34)c2)cc1 ; c1ccc(-c2ccccc2N(c2ccc(-c3ccc4ccccc4c3)cc2)c2cccc3c2oc2c(-c4ccccc4)ccc(-c4ccccc4)c23)cc1]

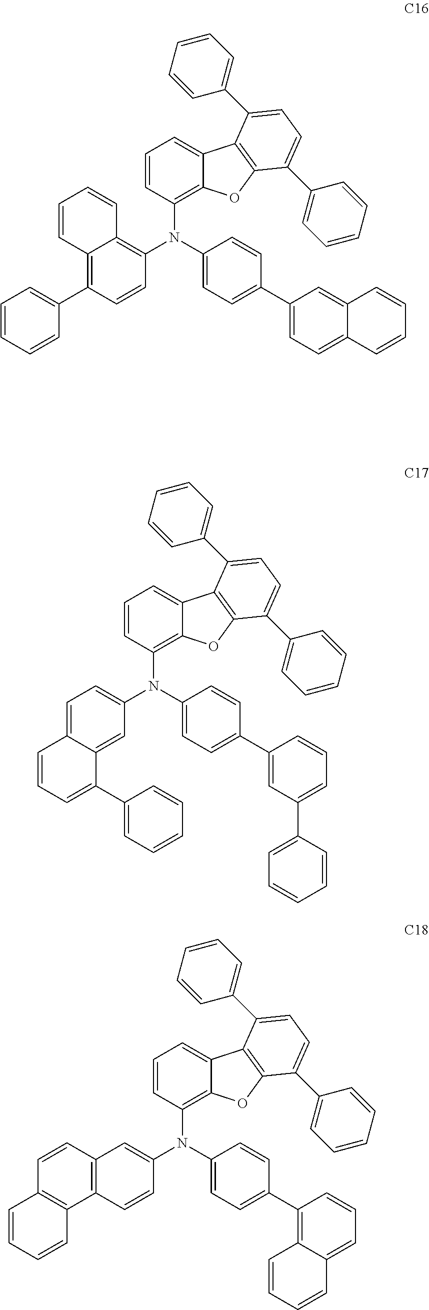 [SMILES: c1ccc(-c2ccc(N(c3ccc(-c4ccc5ccccc5c4)cc3)c3cccc4c3oc3c(-c5ccccc5)ccc(-c5ccccc5)c34)c3ccccc23)cc1 ; c1ccc(-c2cccc(-c3ccc(N(c4ccc5cccc(-c6ccccc6)c5c4)c4cccc5c4oc4c(-c6ccccc6)ccc(-c6ccccc6)c45)cc3)c2)cc1 ; c1ccc(-c2ccc(-c3ccccc3)c3c2oc2c(N(c4ccc(-c5cccc6ccccc56)cc4)c4ccc5c(ccc6ccccc65)c4)cccc23)cc1]

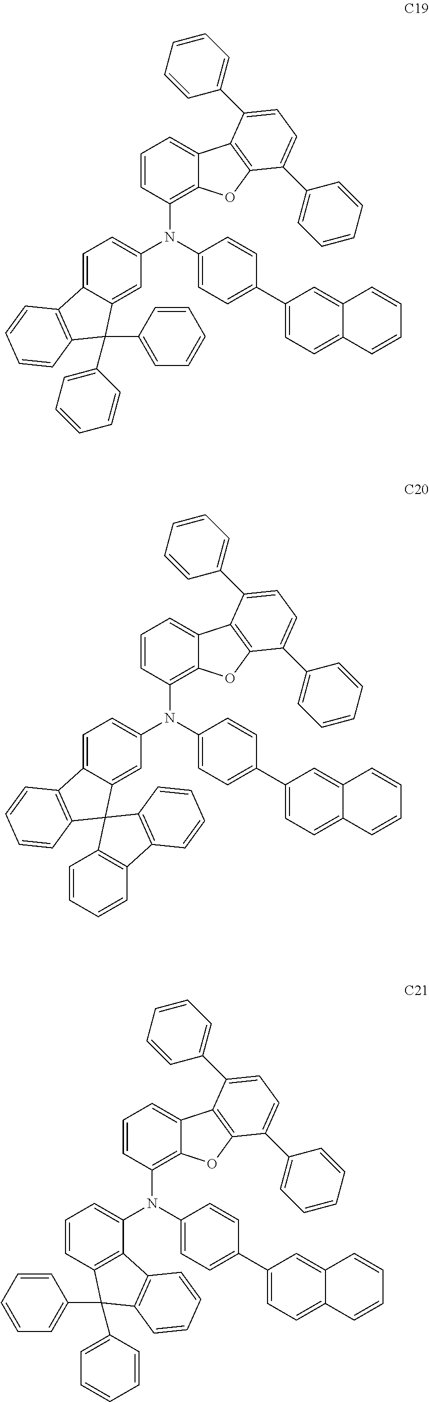 [SMILES: c1ccc(-c2ccc(-c3ccccc3)c3c2oc2c(N(c4ccc(-c5ccc6ccccc6c5)cc4)c4ccc5c(c4)C(c4ccccc4)(c4ccccc4)c4ccccc4-5)cccc23)cc1 ; c1ccc(-c2ccc(-c3ccccc3)c3c2oc2c(N(c4ccc(-c5ccc6ccccc6c5)cc4)c4ccc5c(c4)C4(c6ccccc6-c6ccccc64)c4ccccc4-5)cccc23)cc1 ; c1ccc(-c2ccc(-c3ccccc3)c3c2oc2c(N(c4ccc(-c5ccc6ccccc6c5)cc4)c4cccc5c4-c4ccccc4C5(c4ccccc4)c4ccccc4)cccc23)cc1]

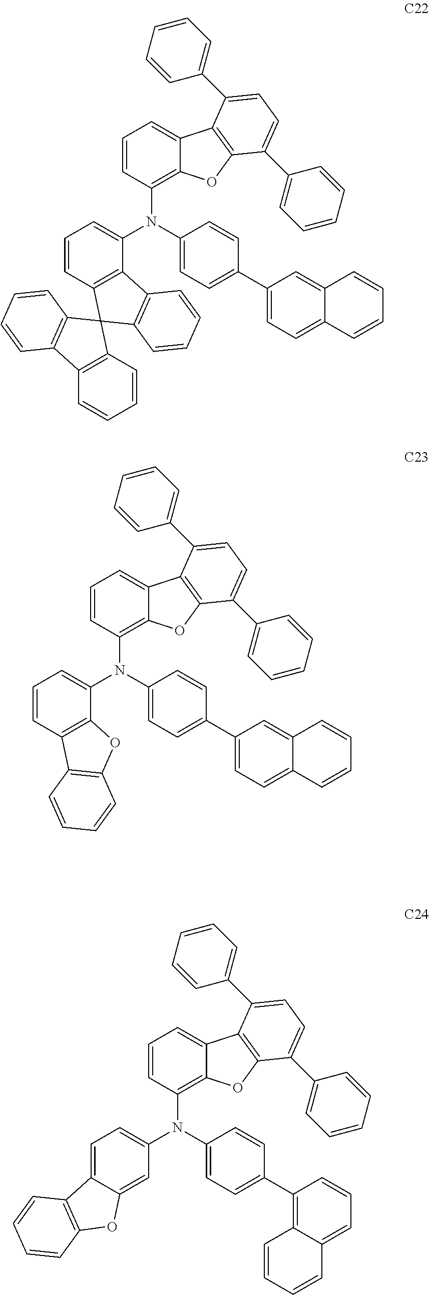 [SMILES: c1ccc(-c2ccc(-c3ccccc3)c3c2oc2c(N(c4ccc(-c5ccc6ccccc6c5)cc4)c4cccc5c4-c4ccccc4C54c5ccccc5-c5ccccc54)cccc23)cc1 ; c1ccc(-c2ccc(-c3ccccc3)c3c2oc2c(N(c4ccc(-c5ccc6ccccc6c5)cc4)c4cccc5c4oc4ccccc45)cccc23)cc1 ; c1ccc(-c2ccc(-c3ccccc3)c3c2oc2c(N(c4ccc(-c5cccc6ccccc56)cc4)c4ccc5c(c4)oc4ccccc45)cccc23)cc1]

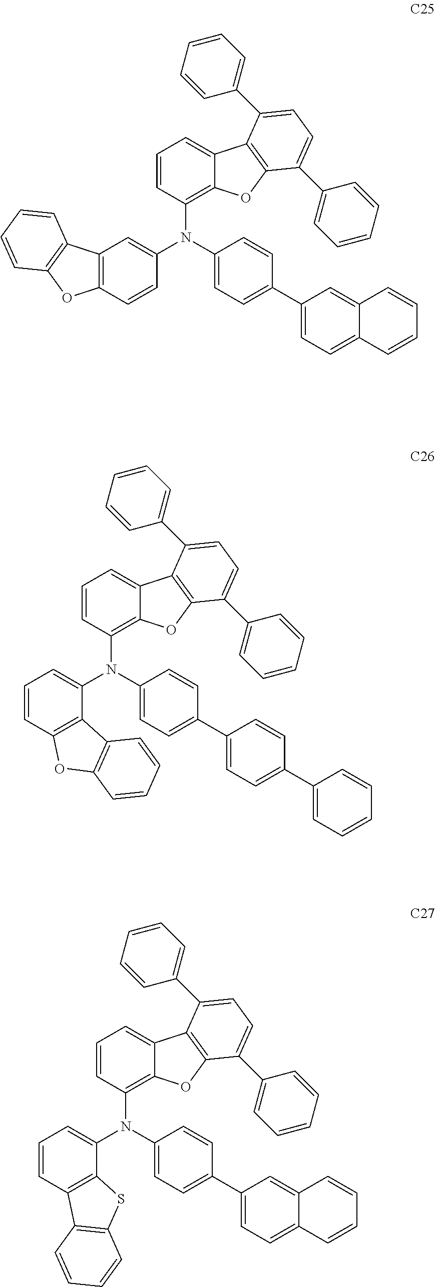 [SMILES: c1ccc(-c2ccc(-c3ccccc3)c3c2oc2c(N(c4ccc(-c5ccc6ccccc6c5)cc4)c4ccc5oc6ccccc6c5c4)cccc23)cc1 ; c1ccc(-c2ccc(-c3ccc(N(c4cccc5c4oc4c(-c6ccccc6)ccc(-c6ccccc6)c45)c4cccc5oc6ccccc6c45)cc3)cc2)cc1 ; c1ccc(-c2ccc(-c3ccccc3)c3c2oc2c(N(c4ccc(-c5ccc6ccccc6c5)cc4)c4cccc5c4sc4ccccc45)cccc23)cc1]

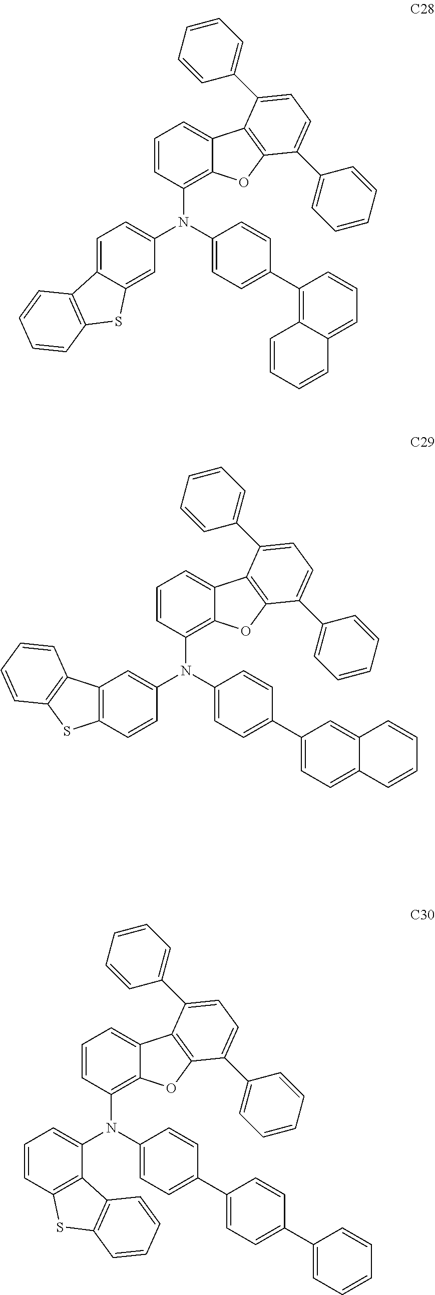 [SMILES: c1ccc(-c2ccc(-c3ccccc3)c3c2oc2c(N(c4ccc(-c5cccc6ccccc56)cc4)c4ccc5c(c4)sc4ccccc45)cccc23)cc1 ; c1ccc(-c2ccc(-c3ccccc3)c3c2oc2c(N(c4ccc(-c5ccc6ccccc6c5)cc4)c4ccc5sc6ccccc6c5c4)cccc23)cc1 ; c1ccc(-c2ccc(-c3ccc(N(c4cccc5c4oc4c(-c6ccccc6)ccc(-c6ccccc6)c45)c4cccc5sc6ccccc6c45)cc3)cc2)cc1]

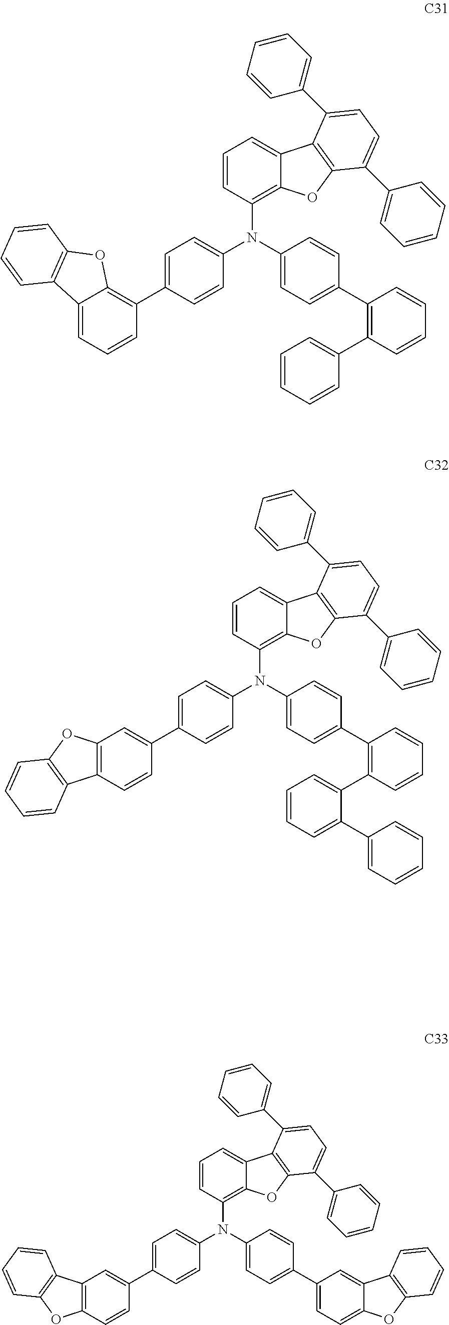 [SMILES: c1ccc(-c2ccccc2-c2ccc(N(c3ccc(-c4cccc5c4oc4ccccc45)cc3)c3cccc4c3oc3c(-c5ccccc5)ccc(-c5ccccc5)c34)cc2)cc1 ; c1ccc(-c2ccccc2-c2ccccc2-c2ccc(N(c3ccc(-c4ccc5c(c4)oc4ccccc45)cc3)c3cccc4c3oc3c(-c5ccccc5)ccc(-c5ccccc5)c34)cc2)cc1 ; c1ccc(-c2ccc(-c3ccccc3)c3c2oc2c(N(c4ccc(-c5ccc6oc7ccccc7c6c5)cc4)c4ccc(-c5ccc6oc7ccccc7c6c5)cc4)cccc23)cc1]

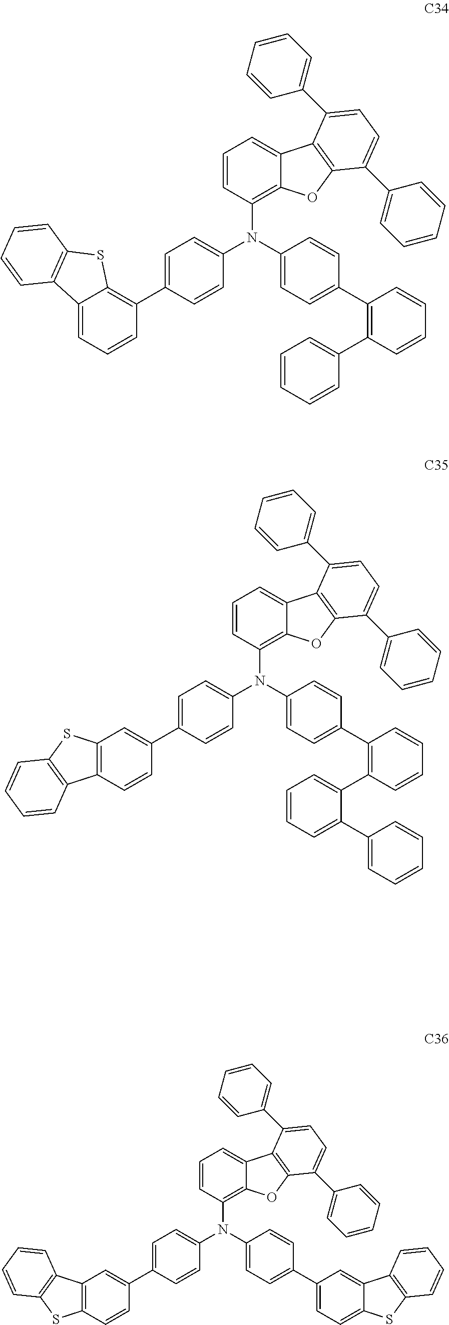 [SMILES: c1ccc(-c2ccccc2-c2ccc(N(c3ccc(-c4cccc5c4sc4ccccc45)cc3)c3cccc4c3oc3c(-c5ccccc5)ccc(-c5ccccc5)c34)cc2)cc1 ; c1ccc(-c2ccccc2-c2ccccc2-c2ccc(N(c3ccc(-c4ccc5c(c4)sc4ccccc45)cc3)c3cccc4c3oc3c(-c5ccccc5)ccc(-c5ccccc5)c34)cc2)cc1 ; c1ccc(-c2ccc(-c3ccccc3)c3c2oc2c(N(c4ccc(-c5ccc6sc7ccccc7c6c5)cc4)c4ccc(-c5ccc6sc7ccccc7c6c5)cc4)cccc23)cc1]

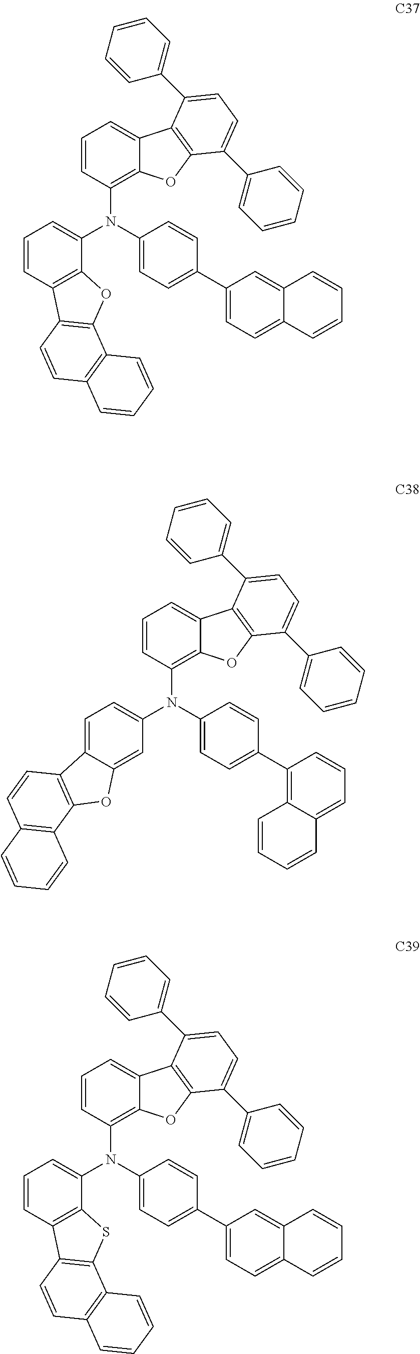 [SMILES: c1ccc(-c2ccc(-c3ccccc3)c3c2oc2c(N(c4ccc(-c5ccc6ccccc6c5)cc4)c4cccc5c4oc4c6ccccc6ccc54)cccc23)cc1 ; c1ccc(-c2ccc(-c3ccccc3)c3c2oc2c(N(c4ccc(-c5cccc6ccccc56)cc4)c4ccc5c(c4)oc4c6ccccc6ccc54)cccc23)cc1 ; c1ccc(-c2ccc(-c3ccccc3)c3c2oc2c(N(c4ccc(-c5ccc6ccccc6c5)cc4)c4cccc5c4sc4c6ccccc6ccc54)cccc23)cc1]

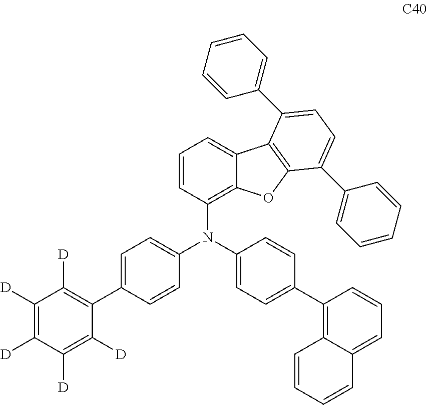 [SMILES: [2H]c1c([2H])c([2H])c(-c2ccc(N(c3ccc(-c4cccc5ccccc45)cc3)c3cccc4c3oc3c(-c5ccccc5)ccc(-c5ccccc5)c34)cc2)c([2H])c1[2H]]

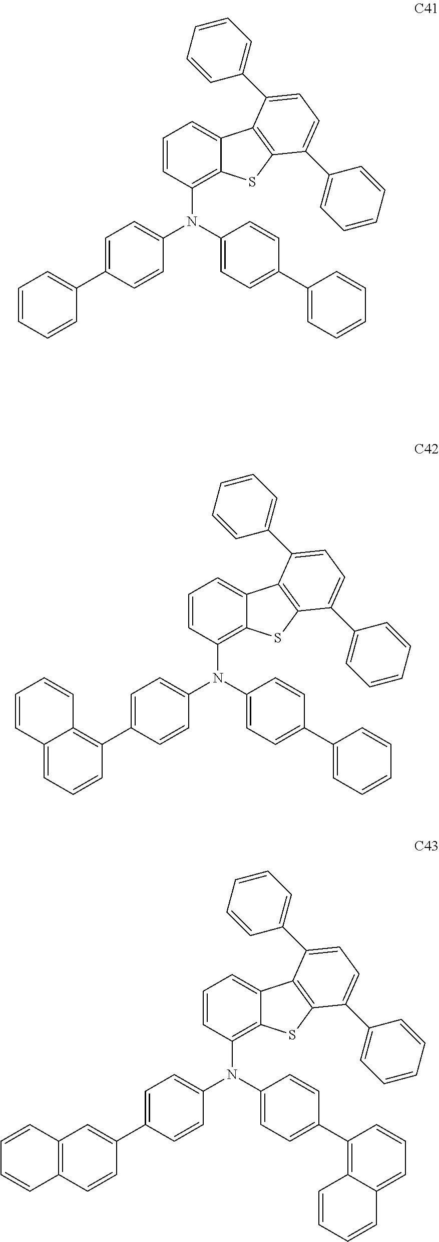 [SMILES: c1ccc(-c2ccc(N(c3ccc(-c4ccccc4)cc3)c3cccc4c3sc3c(-c5ccccc5)ccc(-c5ccccc5)c34)cc2)cc1 ; c1ccc(-c2ccc(N(c3ccc(-c4cccc5ccccc45)cc3)c3cccc4c3sc3c(-c5ccccc5)ccc(-c5ccccc5)c34)cc2)cc1 ; c1ccc(-c2ccc(-c3ccccc3)c3c2sc2c(N(c4ccc(-c5ccc6ccccc6c5)cc4)c4ccc(-c5cccc6ccccc56)cc4)cccc23)cc1]

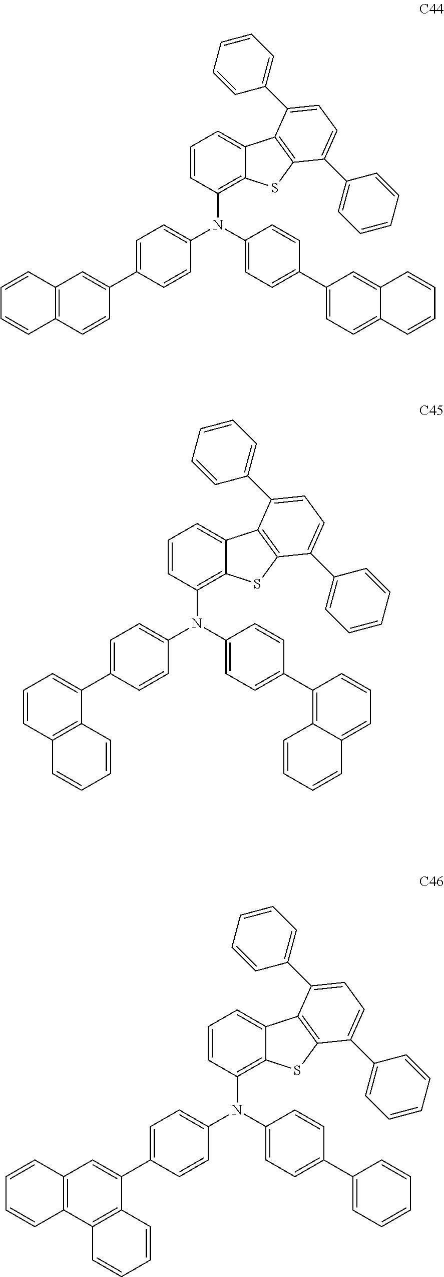 [SMILES: c1ccc(-c2ccc(-c3ccccc3)c3c2sc2c(N(c4ccc(-c5ccc6ccccc6c5)cc4)c4ccc(-c5ccc6ccccc6c5)cc4)cccc23)cc1 ; c1ccc(-c2ccc(-c3ccccc3)c3c2sc2c(N(c4ccc(-c5cccc6ccccc56)cc4)c4ccc(-c5cccc6ccccc56)cc4)cccc23)cc1 ; c1ccc(-c2ccc(N(c3ccc(-c4cc5ccccc5c5ccccc45)cc3)c3cccc4c3sc3c(-c5ccccc5)ccc(-c5ccccc5)c34)cc2)cc1]

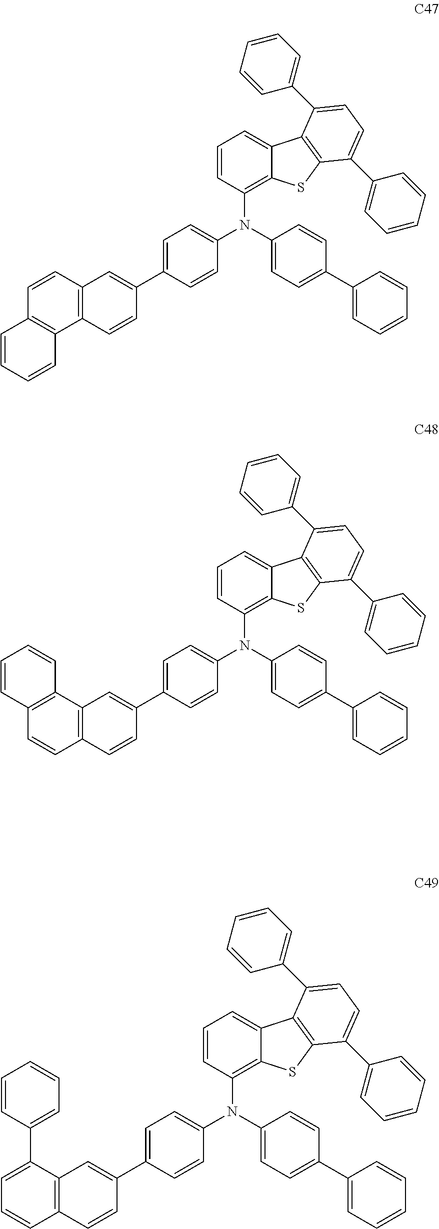 [SMILES: c1ccc(-c2ccc(N(c3ccc(-c4ccc5c(ccc6ccccc65)c4)cc3)c3cccc4c3sc3c(-c5ccccc5)ccc(-c5ccccc5)c34)cc2)cc1 ; c1ccc(-c2ccc(N(c3ccc(-c4ccc5ccc6ccccc6c5c4)cc3)c3cccc4c3sc3c(-c5ccccc5)ccc(-c5ccccc5)c34)cc2)cc1 ; c1ccc(-c2ccc(N(c3ccc(-c4ccc5cccc(-c6ccccc6)c5c4)cc3)c3cccc4c3sc3c(-c5ccccc5)ccc(-c5ccccc5)c34)cc2)cc1]

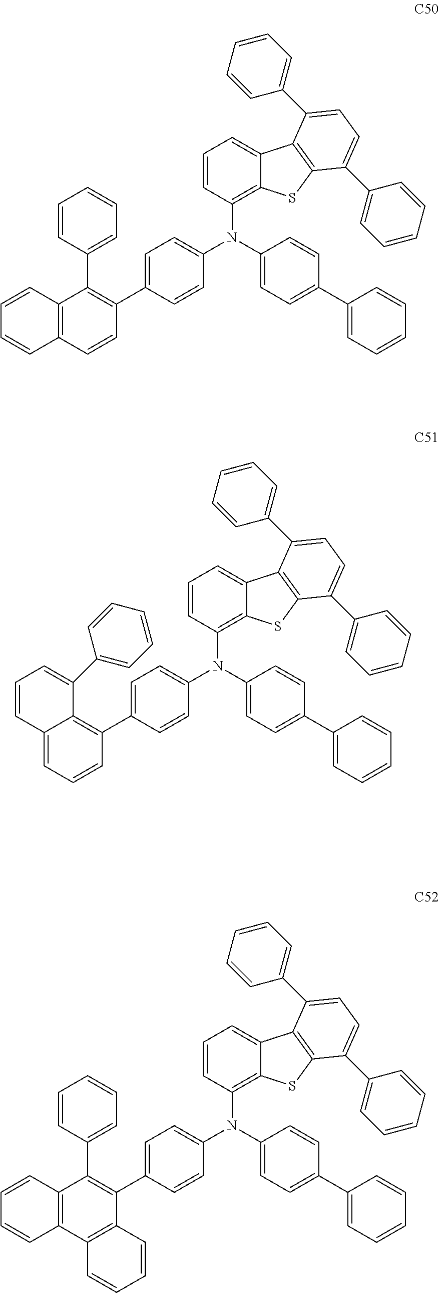 [SMILES: c1ccc(-c2ccc(N(c3ccc(-c4ccc5ccccc5c4-c4ccccc4)cc3)c3cccc4c3sc3c(-c5ccccc5)ccc(-c5ccccc5)c34)cc2)cc1 ; c1ccc(-c2ccc(N(c3ccc(-c4cccc5cccc(-c6ccccc6)c45)cc3)c3cccc4c3sc3c(-c5ccccc5)ccc(-c5ccccc5)c34)cc2)cc1 ; c1ccc(-c2ccc(N(c3ccc(-c4c(-c5ccccc5)c5ccccc5c5ccccc45)cc3)c3cccc4c3sc3c(-c5ccccc5)ccc(-c5ccccc5)c34)cc2)cc1]

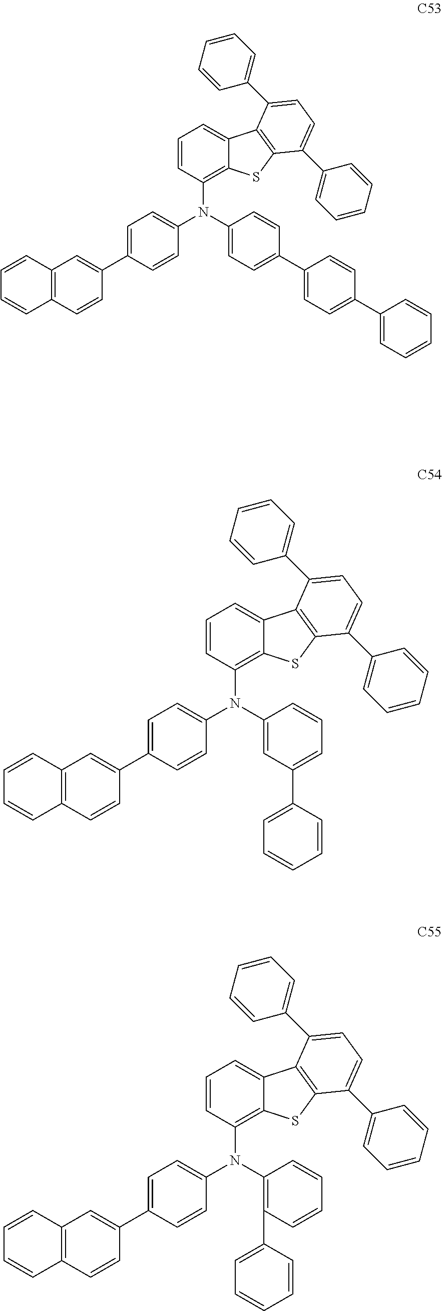 [SMILES: c1ccc(-c2ccc(-c3ccc(N(c4ccc(-c5ccc6ccccc6c5)cc4)c4cccc5c4sc4c(-c6ccccc6)ccc(-c6ccccc6)c45)cc3)cc2)cc1 ; c1ccc(-c2cccc(N(c3ccc(-c4ccc5ccccc5c4)cc3)c3cccc4c3sc3c(-c5ccccc5)ccc(-c5ccccc5)c34)c2)cc1 ; c1ccc(-c2ccccc2N(c2ccc(-c3ccc4ccccc4c3)cc2)c2cccc3c2sc2c(-c4ccccc4)ccc(-c4ccccc4)c23)cc1]

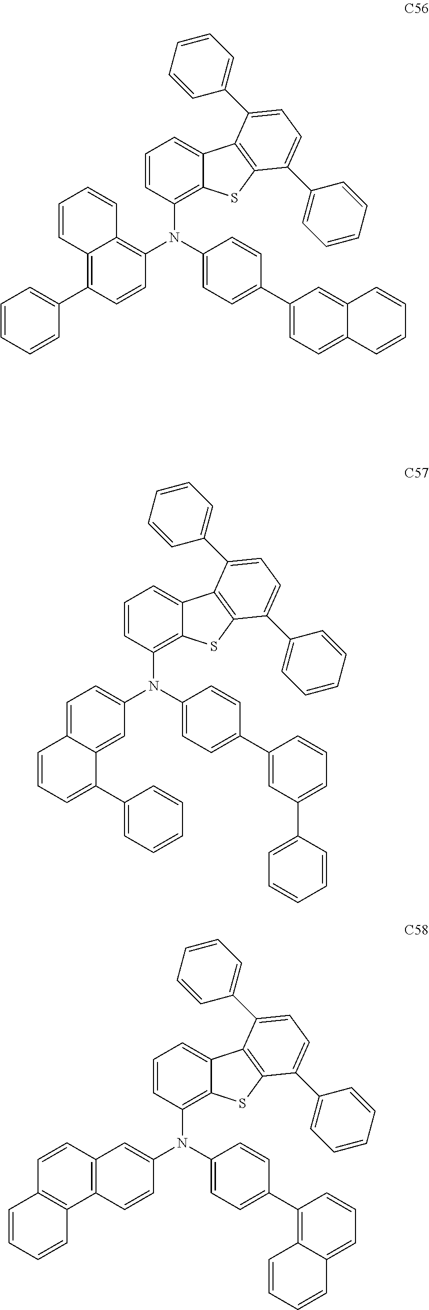 [SMILES: c1ccc(-c2ccc(N(c3ccc(-c4ccc5ccccc5c4)cc3)c3cccc4c3sc3c(-c5ccccc5)ccc(-c5ccccc5)c34)c3ccccc23)cc1 ; c1ccc(-c2cccc(-c3ccc(N(c4ccc5cccc(-c6ccccc6)c5c4)c4cccc5c4sc4c(-c6ccccc6)ccc(-c6ccccc6)c45)cc3)c2)cc1 ; c1ccc(-c2ccc(-c3ccccc3)c3c2sc2c(N(c4ccc(-c5cccc6ccccc56)cc4)c4ccc5c(ccc6ccccc65)c4)cccc23)cc1]

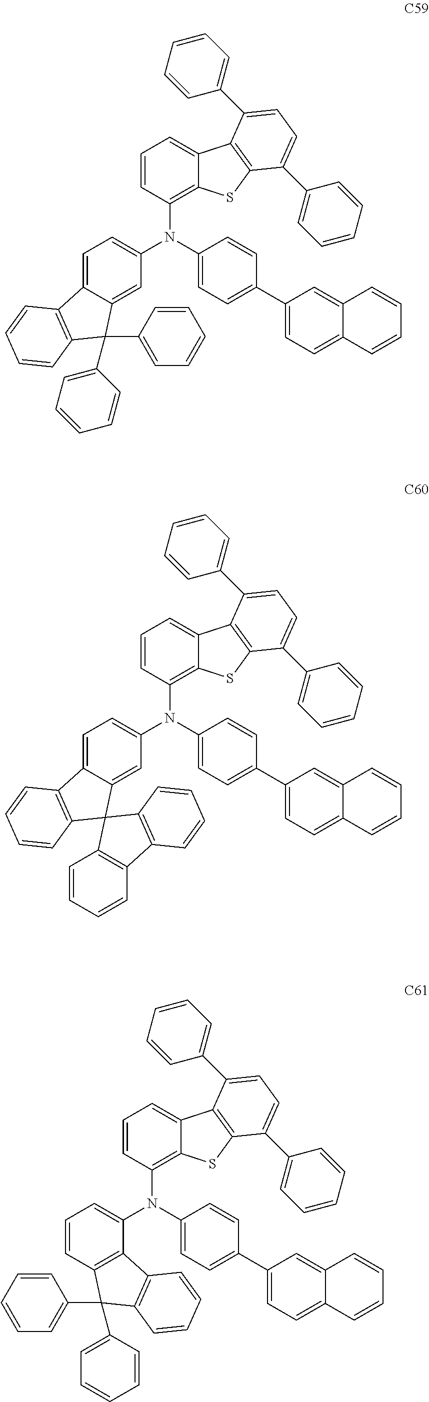 [SMILES: c1ccc(-c2ccc(-c3ccccc3)c3c2sc2c(N(c4ccc(-c5ccc6ccccc6c5)cc4)c4ccc5c(c4)C(c4ccccc4)(c4ccccc4)c4ccccc4-5)cccc23)cc1 ; c1ccc(-c2ccc(-c3ccccc3)c3c2sc2c(N(c4ccc(-c5ccc6ccccc6c5)cc4)c4ccc5c(c4)C4(c6ccccc6-c6ccccc64)c4ccccc4-5)cccc23)cc1 ; c1ccc(-c2ccc(-c3ccccc3)c3c2sc2c(N(c4ccc(-c5ccc6ccccc6c5)cc4)c4cccc5c4-c4ccccc4C5(c4ccccc4)c4ccccc4)cccc23)cc1]

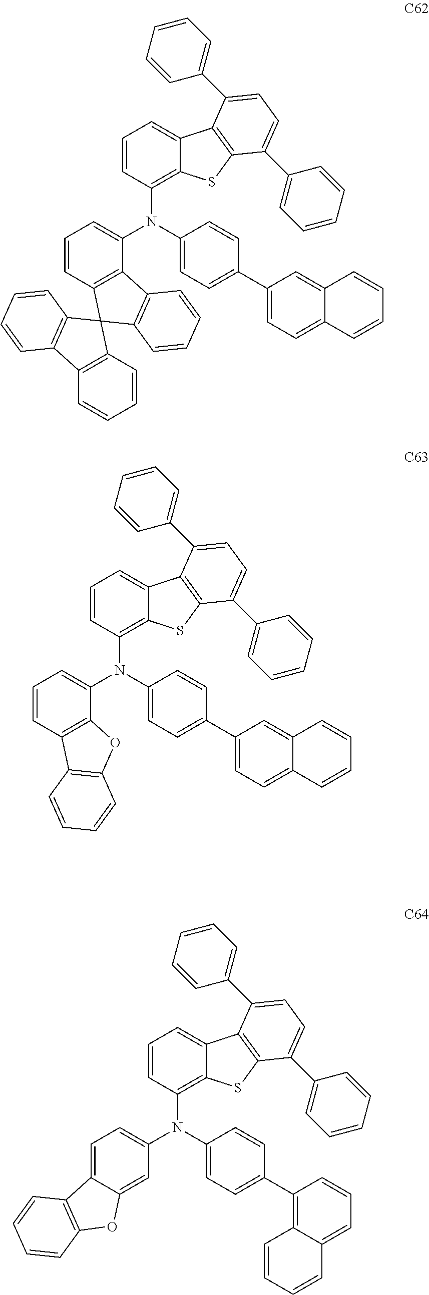 [SMILES: c1ccc(-c2ccc(-c3ccccc3)c3c2sc2c(N(c4ccc(-c5ccc6ccccc6c5)cc4)c4cccc5c4-c4ccccc4C54c5ccccc5-c5ccccc54)cccc23)cc1 ; c1ccc(-c2ccc(-c3ccccc3)c3c2sc2c(N(c4ccc(-c5ccc6ccccc6c5)cc4)c4cccc5c4oc4ccccc45)cccc23)cc1 ; c1ccc(-c2ccc(-c3ccccc3)c3c2sc2c(N(c4ccc(-c5cccc6ccccc56)cc4)c4ccc5c(c4)oc4ccccc45)cccc23)cc1]

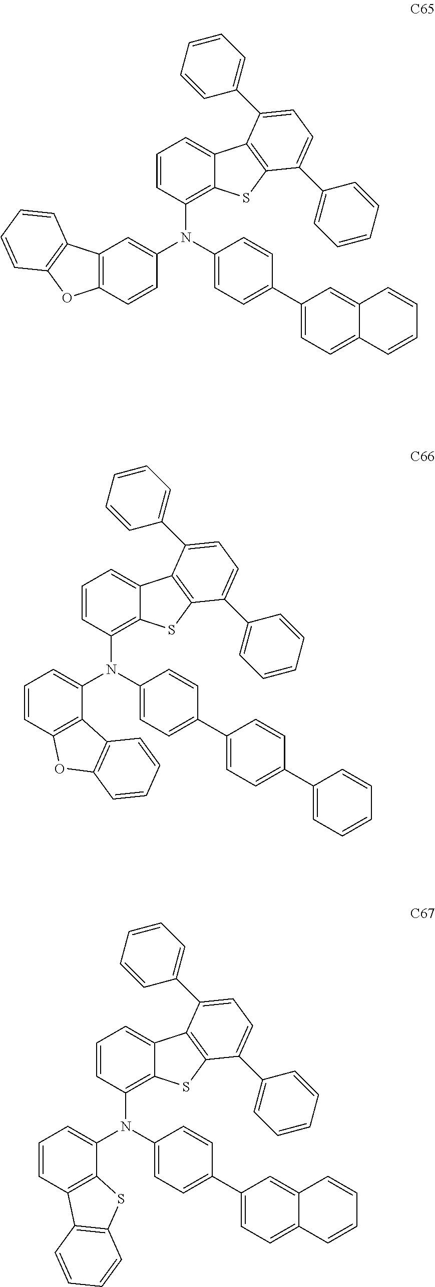 [SMILES: c1ccc(-c2ccc(-c3ccccc3)c3c2sc2c(N(c4ccc(-c5ccc6ccccc6c5)cc4)c4ccc5oc6ccccc6c5c4)cccc23)cc1 ; c1ccc(-c2ccc(-c3ccc(N(c4cccc5c4sc4c(-c6ccccc6)ccc(-c6ccccc6)c45)c4cccc5oc6ccccc6c45)cc3)cc2)cc1 ; c1ccc(-c2ccc(-c3ccccc3)c3c2sc2c(N(c4ccc(-c5ccc6ccccc6c5)cc4)c4cccc5c4sc4ccccc45)cccc23)cc1]

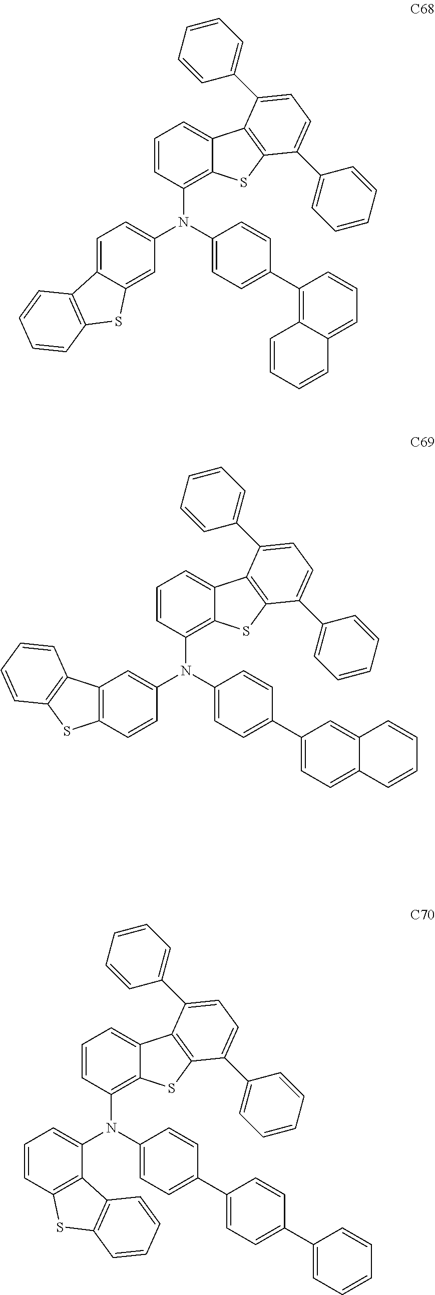 [SMILES: c1ccc(-c2ccc(-c3ccccc3)c3c2sc2c(N(c4ccc(-c5cccc6ccccc56)cc4)c4ccc5c(c4)sc4ccccc45)cccc23)cc1 ; c1ccc(-c2ccc(-c3ccccc3)c3c2sc2c(N(c4ccc(-c5ccc6ccccc6c5)cc4)c4ccc5sc6ccccc6c5c4)cccc23)cc1 ; c1ccc(-c2ccc(-c3ccc(N(c4cccc5c4sc4c(-c6ccccc6)ccc(-c6ccccc6)c45)c4cccc5sc6ccccc6c45)cc3)cc2)cc1]

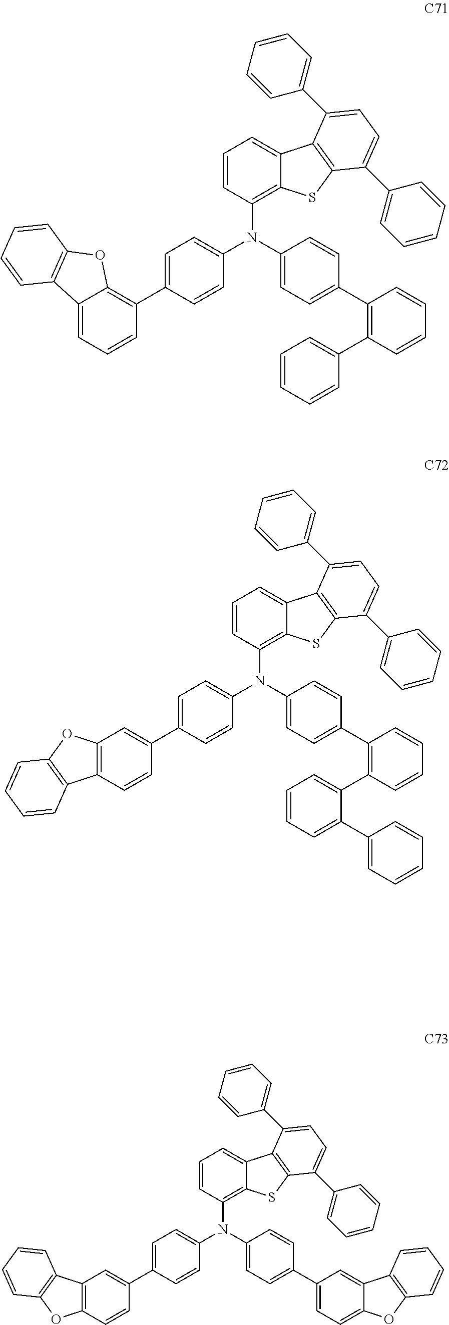 [SMILES: c1ccc(-c2ccccc2-c2ccc(N(c3ccc(-c4cccc5c4oc4ccccc45)cc3)c3cccc4c3sc3c(-c5ccccc5)ccc(-c5ccccc5)c34)cc2)cc1 ; c1ccc(-c2ccccc2-c2ccccc2-c2ccc(N(c3ccc(-c4ccc5c(c4)oc4ccccc45)cc3)c3cccc4c3sc3c(-c5ccccc5)ccc(-c5ccccc5)c34)cc2)cc1 ; c1ccc(-c2ccc(-c3ccccc3)c3c2sc2c(N(c4ccc(-c5ccc6oc7ccccc7c6c5)cc4)c4ccc(-c5ccc6oc7ccccc7c6c5)cc4)cccc23)cc1]

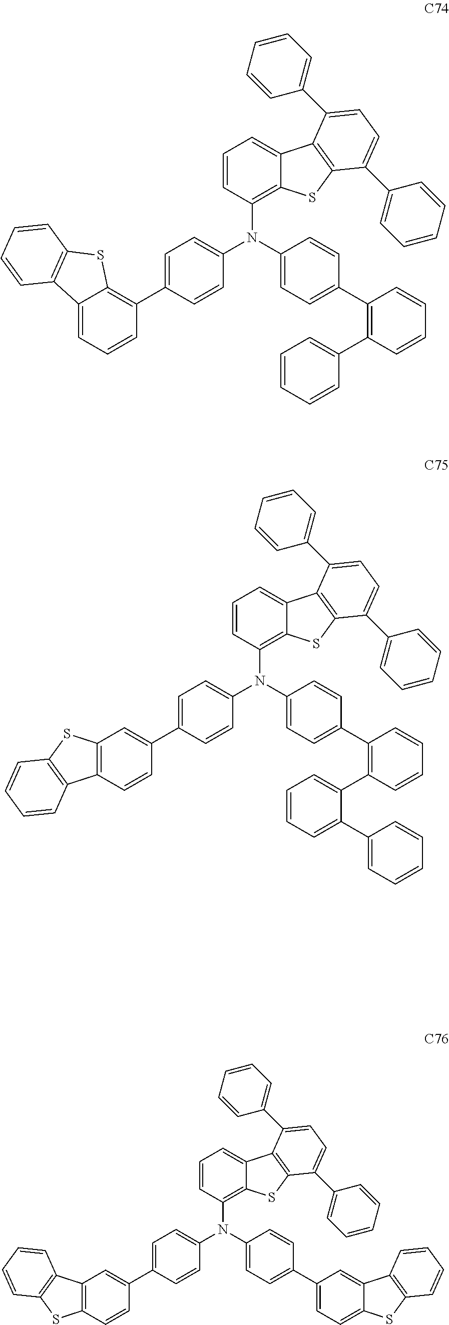 [SMILES: c1ccc(-c2ccccc2-c2ccc(N(c3ccc(-c4cccc5c4sc4ccccc45)cc3)c3cccc4c3sc3c(-c5ccccc5)ccc(-c5ccccc5)c34)cc2)cc1 ; c1ccc(-c2ccccc2-c2ccccc2-c2ccc(N(c3ccc(-c4ccc5c(c4)sc4ccccc45)cc3)c3cccc4c3sc3c(-c5ccccc5)ccc(-c5ccccc5)c34)cc2)cc1 ; c1ccc(-c2ccc(-c3ccccc3)c3c2sc2c(N(c4ccc(-c5ccc6sc7ccccc7c6c5)cc4)c4ccc(-c5ccc6sc7ccccc7c6c5)cc4)cccc23)cc1]

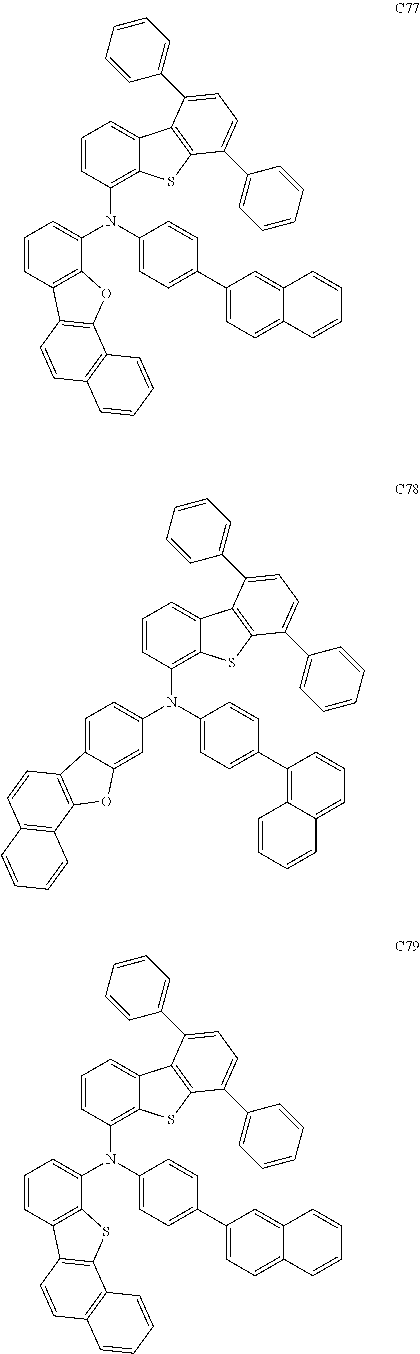 [SMILES: c1ccc(-c2ccc(-c3ccccc3)c3c2sc2c(N(c4ccc(-c5ccc6ccccc6c5)cc4)c4cccc5c4oc4c6ccccc6ccc54)cccc23)cc1 ; c1ccc(-c2ccc(-c3ccccc3)c3c2sc2c(N(c4ccc(-c5cccc6ccccc56)cc4)c4ccc5c(c4)oc4c6ccccc6ccc54)cccc23)cc1 ; c1ccc(-c2ccc(-c3ccccc3)c3c2sc2c(N(c4ccc(-c5ccc6ccccc6c5)cc4)c4cccc5c4sc4c6ccccc6ccc54)cccc23)cc1]

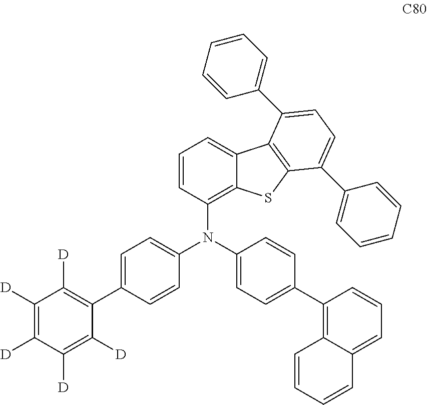 [SMILES: [2H]c1c([2H])c([2H])c(-c2ccc(N(c3ccc(-c4cccc5ccccc45)cc3)c3cccc4c3sc3c(-c5ccccc5)ccc(-c5ccccc5)c34)cc2)c([2H])c1[2H]]

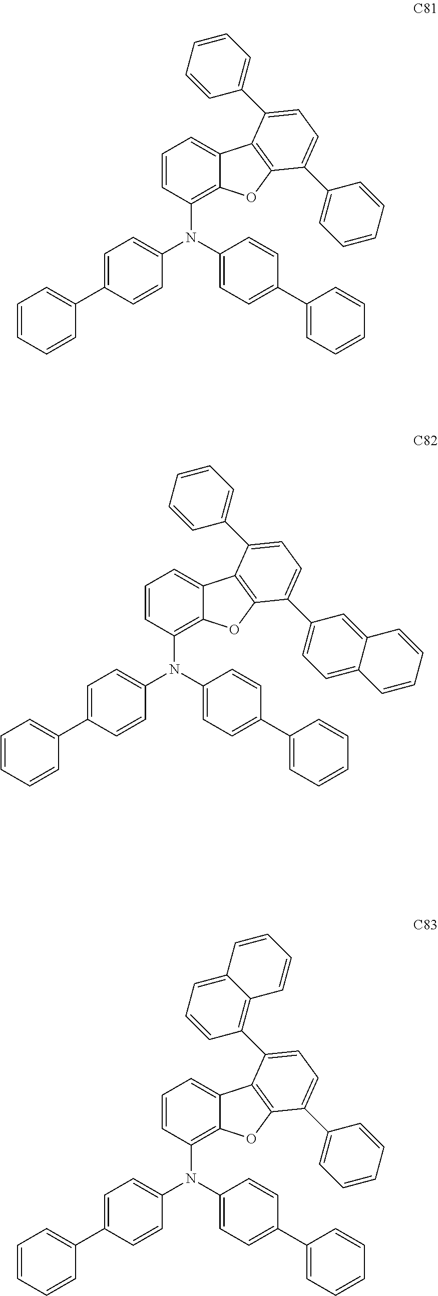 [SMILES: c1ccc(-c2ccc(N(c3ccc(-c4ccccc4)cc3)c3cccc4c3oc3c(-c5ccccc5)ccc(-c5ccccc5)c34)cc2)cc1 ; c1ccc(-c2ccc(N(c3ccc(-c4ccccc4)cc3)c3cccc4c3oc3c(-c5ccc6ccccc6c5)ccc(-c5ccccc5)c34)cc2)cc1 ; c1ccc(-c2ccc(N(c3ccc(-c4ccccc4)cc3)c3cccc4c3oc3c(-c5ccccc5)ccc(-c5cccc6ccccc56)c34)cc2)cc1]

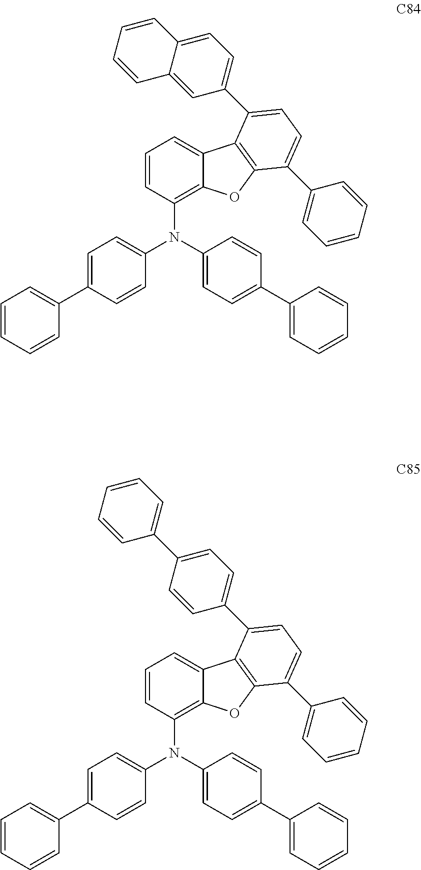 [SMILES: c1ccc(-c2ccc(N(c3ccc(-c4ccccc4)cc3)c3cccc4c3oc3c(-c5ccccc5)ccc(-c5ccc6ccccc6c5)c34)cc2)cc1 ; c1ccc(-c2ccc(-c3ccc(-c4ccccc4)c4oc5c(N(c6ccc(-c7ccccc7)cc6)c6ccc(-c7ccccc7)cc6)cccc5c34)cc2)cc1]

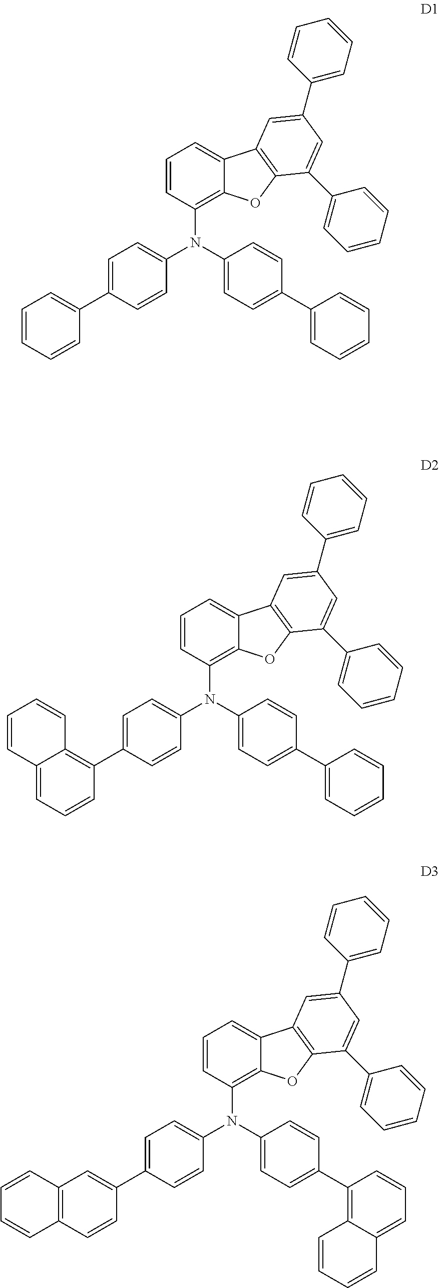 [SMILES: c1ccc(-c2ccc(N(c3ccc(-c4ccccc4)cc3)c3cccc4c3oc3c(-c5ccccc5)cc(-c5ccccc5)cc34)cc2)cc1 ; c1ccc(-c2ccc(N(c3ccc(-c4cccc5ccccc45)cc3)c3cccc4c3oc3c(-c5ccccc5)cc(-c5ccccc5)cc34)cc2)cc1 ; c1ccc(-c2cc(-c3ccccc3)c3oc4c(N(c5ccc(-c6ccc7ccccc7c6)cc5)c5ccc(-c6cccc7ccccc67)cc5)cccc4c3c2)cc1]

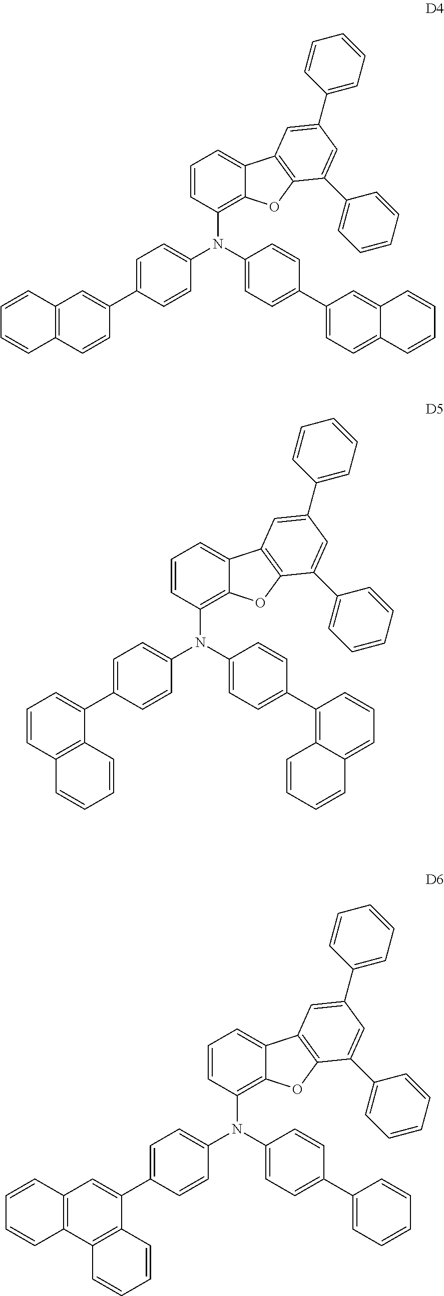 [SMILES: c1ccc(-c2cc(-c3ccccc3)c3oc4c(N(c5ccc(-c6ccc7ccccc7c6)cc5)c5ccc(-c6ccc7ccccc7c6)cc5)cccc4c3c2)cc1 ; c1ccc(-c2cc(-c3ccccc3)c3oc4c(N(c5ccc(-c6cccc7ccccc67)cc5)c5ccc(-c6cccc7ccccc67)cc5)cccc4c3c2)cc1 ; c1ccc(-c2ccc(N(c3ccc(-c4cc5ccccc5c5ccccc45)cc3)c3cccc4c3oc3c(-c5ccccc5)cc(-c5ccccc5)cc34)cc2)cc1]

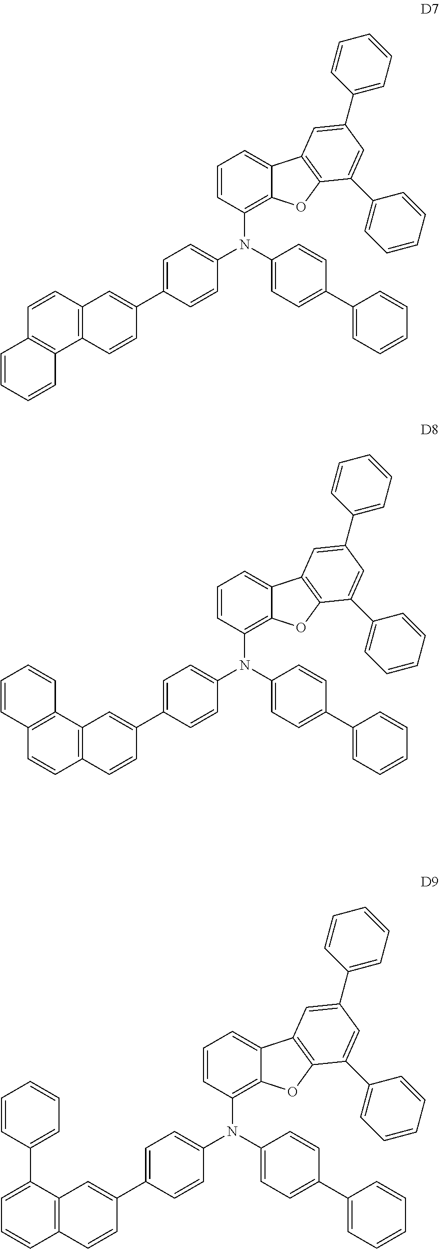 [SMILES: c1ccc(-c2ccc(N(c3ccc(-c4ccc5c(ccc6ccccc65)c4)cc3)c3cccc4c3oc3c(-c5ccccc5)cc(-c5ccccc5)cc34)cc2)cc1 ; c1ccc(-c2ccc(N(c3ccc(-c4ccc5ccc6ccccc6c5c4)cc3)c3cccc4c3oc3c(-c5ccccc5)cc(-c5ccccc5)cc34)cc2)cc1 ; c1ccc(-c2ccc(N(c3ccc(-c4ccc5cccc(-c6ccccc6)c5c4)cc3)c3cccc4c3oc3c(-c5ccccc5)cc(-c5ccccc5)cc34)cc2)cc1]

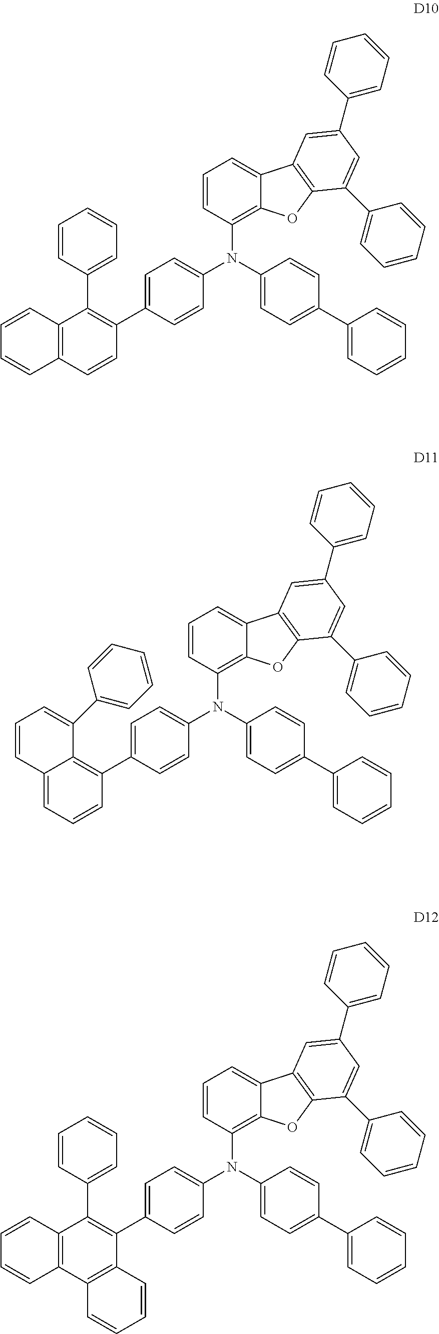 [SMILES: c1ccc(-c2ccc(N(c3ccc(-c4ccc5ccccc5c4-c4ccccc4)cc3)c3cccc4c3oc3c(-c5ccccc5)cc(-c5ccccc5)cc34)cc2)cc1 ; c1ccc(-c2ccc(N(c3ccc(-c4cccc5cccc(-c6ccccc6)c45)cc3)c3cccc4c3oc3c(-c5ccccc5)cc(-c5ccccc5)cc34)cc2)cc1 ; c1ccc(-c2ccc(N(c3ccc(-c4c(-c5ccccc5)c5ccccc5c5ccccc45)cc3)c3cccc4c3oc3c(-c5ccccc5)cc(-c5ccccc5)cc34)cc2)cc1]

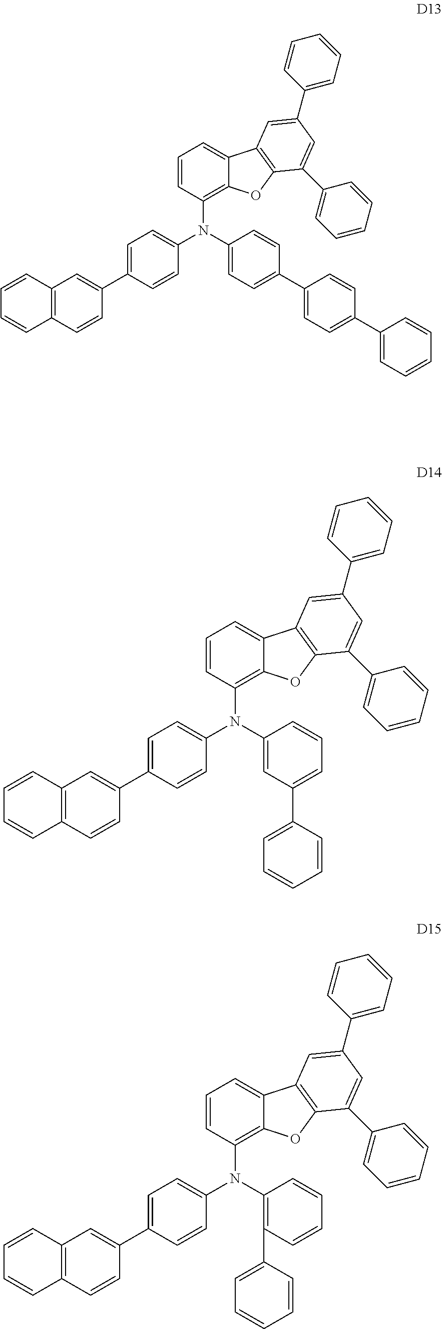 [SMILES: c1ccc(-c2ccc(-c3ccc(N(c4ccc(-c5ccc6ccccc6c5)cc4)c4cccc5c4oc4c(-c6ccccc6)cc(-c6ccccc6)cc45)cc3)cc2)cc1 ; c1ccc(-c2cccc(N(c3ccc(-c4ccc5ccccc5c4)cc3)c3cccc4c3oc3c(-c5ccccc5)cc(-c5ccccc5)cc34)c2)cc1 ; c1ccc(-c2cc(-c3ccccc3)c3oc4c(N(c5ccc(-c6ccc7ccccc7c6)cc5)c5ccccc5-c5ccccc5)cccc4c3c2)cc1]

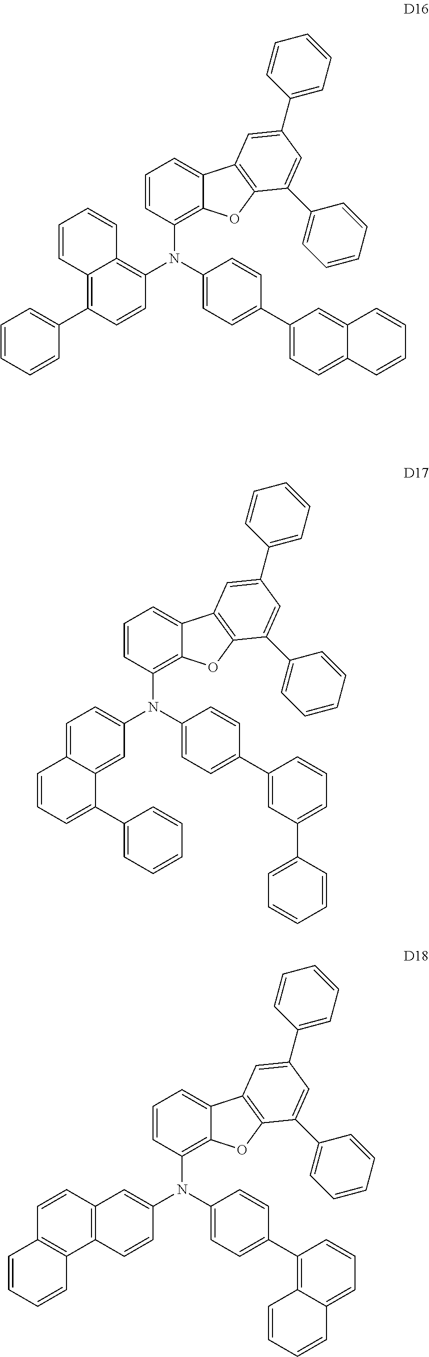

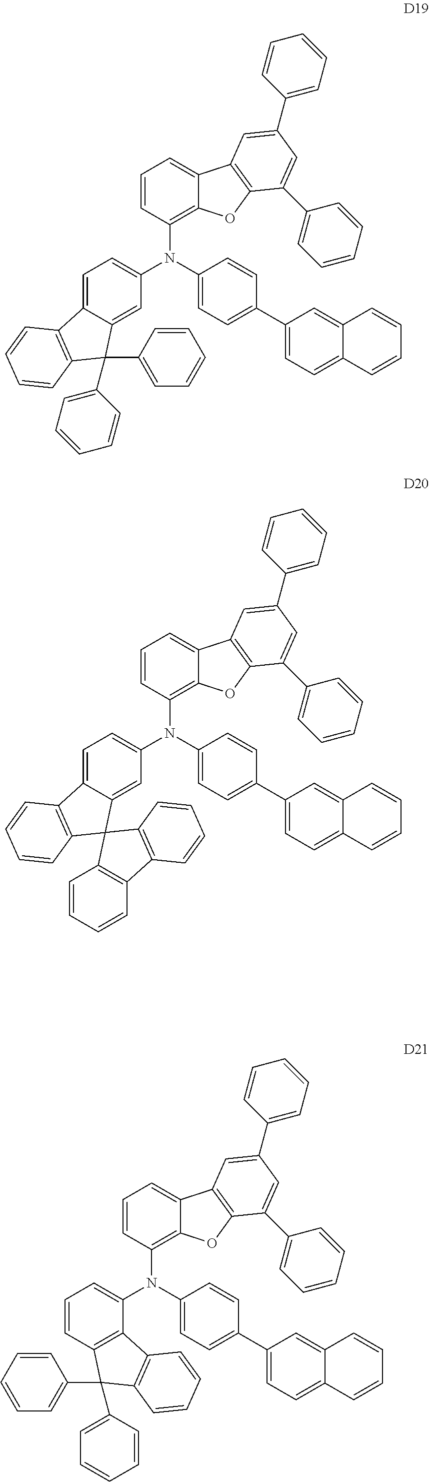 [SMILES: c1ccc(-c2cc(-c3ccccc3)c3oc4c(N(c5ccc(-c6ccc7ccccc7c6)cc5)c5ccc6c(c5)C(c5ccccc5)(c5ccccc5)c5ccccc5-6)cccc4c3c2)cc1 ; c1ccc(-c2cc(-c3ccccc3)c3oc4c(N(c5ccc(-c6ccc7ccccc7c6)cc5)c5ccc6c(c5)C5(c7ccccc7-c7ccccc75)c5ccccc5-6)cccc4c3c2)cc1 ; c1ccc(-c2cc(-c3ccccc3)c3oc4c(N(c5ccc(-c6ccc7ccccc7c6)cc5)c5cccc6c5-c5ccccc5C6(c5ccccc5)c5ccccc5)cccc4c3c2)cc1]

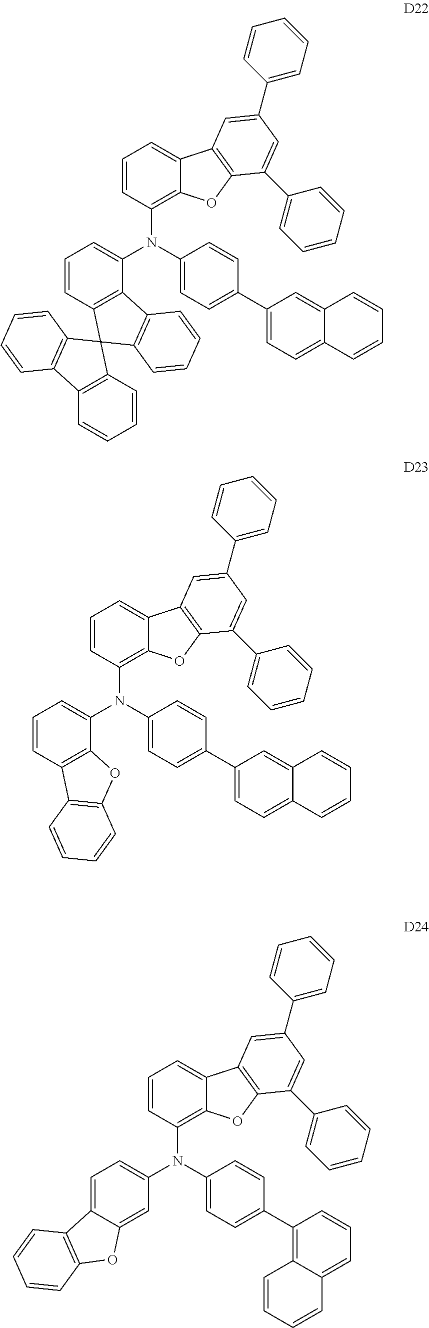 [SMILES: c1ccc(-c2cc(-c3ccccc3)c3oc4c(N(c5ccc(-c6ccc7ccccc7c6)cc5)c5cccc6c5-c5ccccc5C65c6ccccc6-c6ccccc65)cccc4c3c2)cc1 ; c1ccc(-c2cc(-c3ccccc3)c3oc4c(N(c5ccc(-c6ccc7ccccc7c6)cc5)c5cccc6c5oc5ccccc56)cccc4c3c2)cc1 ; c1ccc(-c2cc(-c3ccccc3)c3oc4c(N(c5ccc(-c6cccc7ccccc67)cc5)c5ccc6c(c5)oc5ccccc56)cccc4c3c2)cc1]

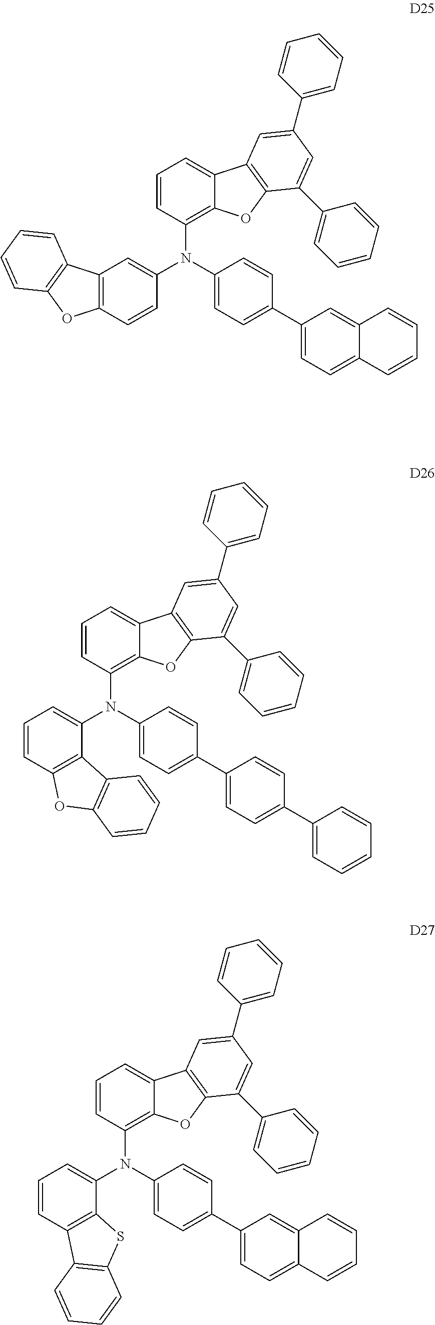 [SMILES: c1ccc(-c2cc(-c3ccccc3)c3oc4c(N(c5ccc(-c6ccc7ccccc7c6)cc5)c5ccc6oc7ccccc7c6c5)cccc4c3c2)cc1 ; c1ccc(-c2ccc(-c3ccc(N(c4cccc5c4oc4c(-c6ccccc6)cc(-c6ccccc6)cc45)c4cccc5oc6ccccc6c45)cc3)cc2)cc1 ; c1ccc(-c2cc(-c3ccccc3)c3oc4c(N(c5ccc(-c6ccc7ccccc7c6)cc5)c5cccc6c5sc5ccccc56)cccc4c3c2)cc1]

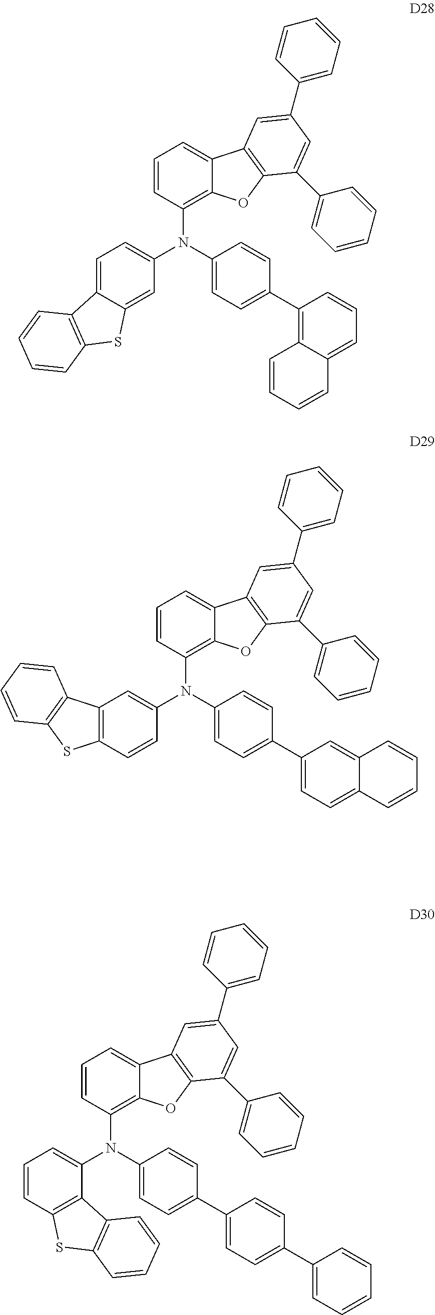 [SMILES: c1ccc(-c2cc(-c3ccccc3)c3oc4c(N(c5ccc(-c6cccc7ccccc67)cc5)c5ccc6c(c5)sc5ccccc56)cccc4c3c2)cc1 ; c1ccc(-c2cc(-c3ccccc3)c3oc4c(N(c5ccc(-c6ccc7ccccc7c6)cc5)c5ccc6sc7ccccc7c6c5)cccc4c3c2)cc1 ; c1ccc(-c2ccc(-c3ccc(N(c4cccc5c4oc4c(-c6ccccc6)cc(-c6ccccc6)cc45)c4cccc5sc6ccccc6c45)cc3)cc2)cc1]

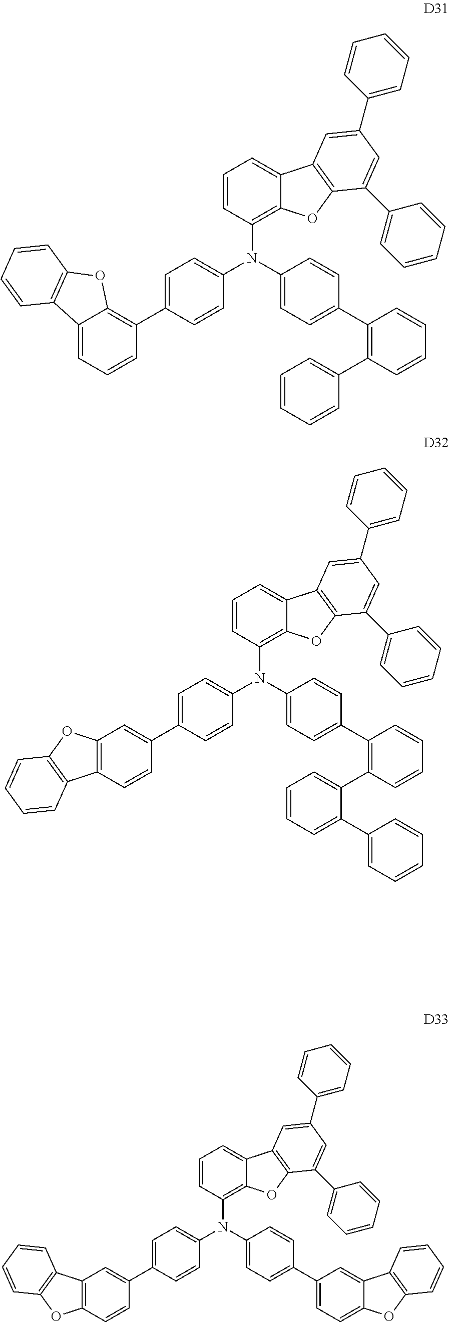 [SMILES: c1ccc(-c2cc(-c3ccccc3)c3oc4c(N(c5ccc(-c6ccccc6-c6ccccc6)cc5)c5ccc(-c6cccc7c6oc6ccccc67)cc5)cccc4c3c2)cc1 ; c1ccc(-c2cc(-c3ccccc3)c3oc4c(N(c5ccc(-c6ccc7c(c6)oc6ccccc67)cc5)c5ccc(-c6ccccc6-c6ccccc6-c6ccccc6)cc5)cccc4c3c2)cc1 ; c1ccc(-c2cc(-c3ccccc3)c3oc4c(N(c5ccc(-c6ccc7oc8ccccc8c7c6)cc5)c5ccc(-c6ccc7oc8ccccc8c7c6)cc5)cccc4c3c2)cc1]

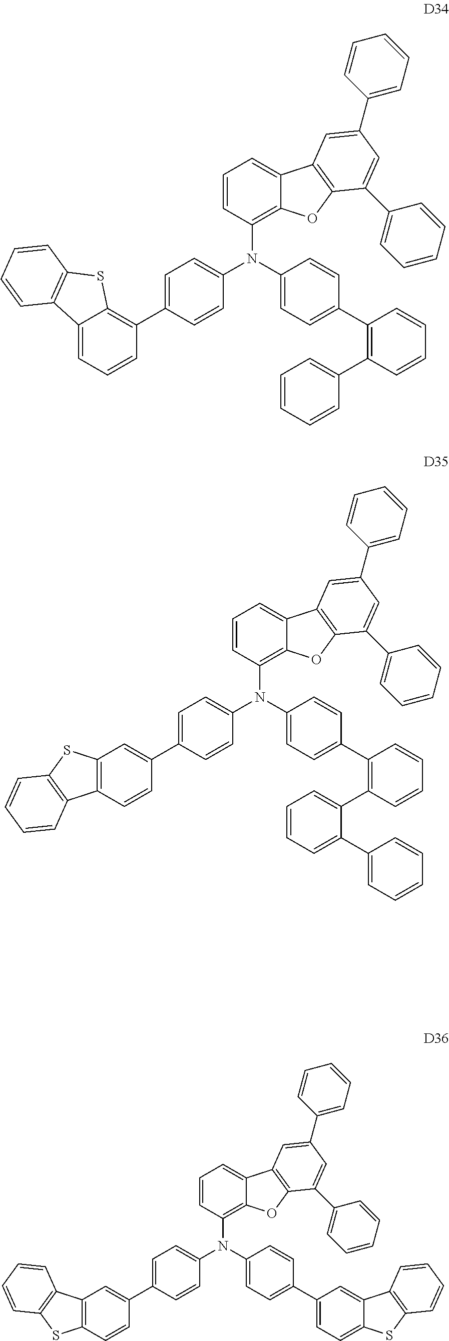 [SMILES: c1ccc(-c2cc(-c3ccccc3)c3oc4c(N(c5ccc(-c6ccccc6-c6ccccc6)cc5)c5ccc(-c6cccc7c6sc6ccccc67)cc5)cccc4c3c2)cc1 ; c1ccc(-c2cc(-c3ccccc3)c3oc4c(N(c5ccc(-c6ccc7c(c6)sc6ccccc67)cc5)c5ccc(-c6ccccc6-c6ccccc6-c6ccccc6)cc5)cccc4c3c2)cc1 ; c1ccc(-c2cc(-c3ccccc3)c3oc4c(N(c5ccc(-c6ccc7sc8ccccc8c7c6)cc5)c5ccc(-c6ccc7sc8ccccc8c7c6)cc5)cccc4c3c2)cc1]

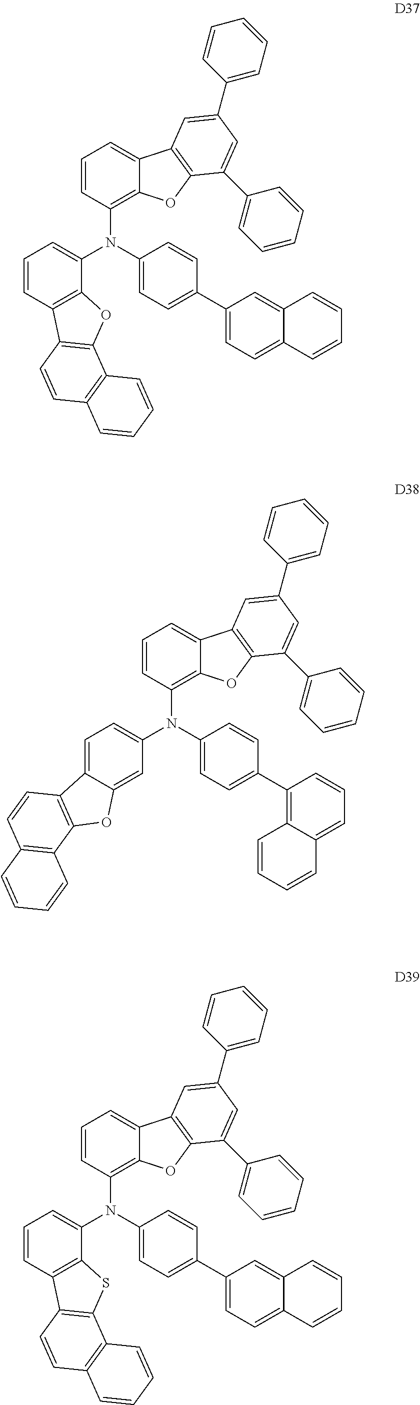 [SMILES: c1ccc(-c2cc(-c3ccccc3)c3oc4c(N(c5ccc(-c6ccc7ccccc7c6)cc5)c5cccc6c5oc5c7ccccc7ccc65)cccc4c3c2)cc1 ; c1ccc(-c2cc(-c3ccccc3)c3oc4c(N(c5ccc(-c6cccc7ccccc67)cc5)c5ccc6c(c5)oc5c7ccccc7ccc65)cccc4c3c2)cc1 ; c1ccc(-c2cc(-c3ccccc3)c3oc4c(N(c5ccc(-c6ccc7ccccc7c6)cc5)c5cccc6c5sc5c7ccccc7ccc65)cccc4c3c2)cc1]

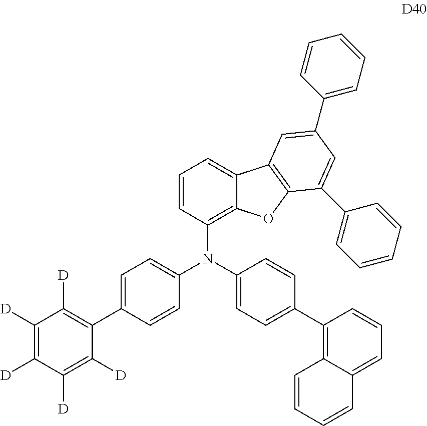 [SMILES: [2H]c1c([2H])c([2H])c(-c2ccc(N(c3ccc(-c4cccc5ccccc45)cc3)c3cccc4c3oc3c(-c5ccccc5)cc(-c5ccccc5)cc34)cc2)c([2H])c1[2H]]

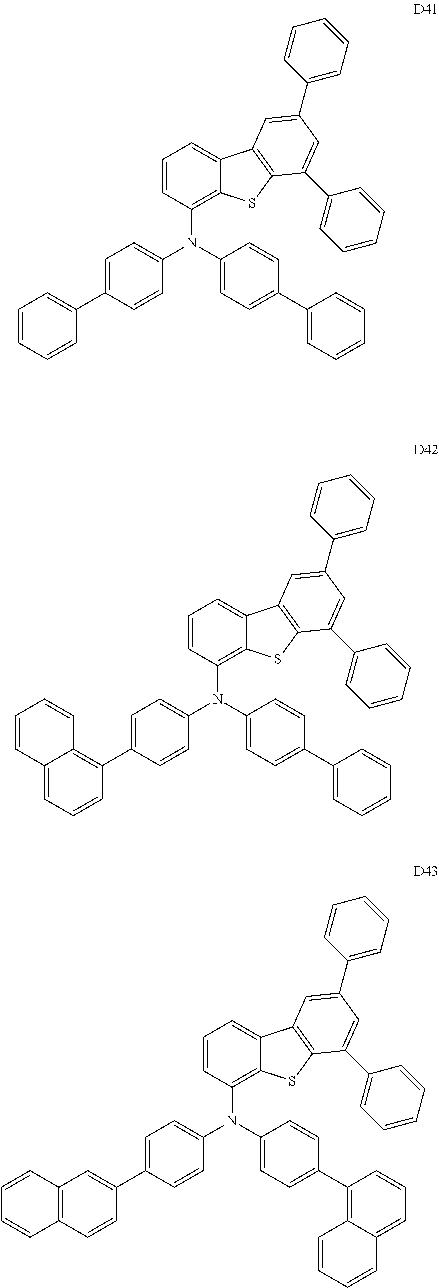 [SMILES: c1ccc(-c2ccc(N(c3ccc(-c4ccccc4)cc3)c3cccc4c3sc3c(-c5ccccc5)cc(-c5ccccc5)cc34)cc2)cc1 ; c1ccc(-c2ccc(N(c3ccc(-c4cccc5ccccc45)cc3)c3cccc4c3sc3c(-c5ccccc5)cc(-c5ccccc5)cc34)cc2)cc1 ; c1ccc(-c2cc(-c3ccccc3)c3sc4c(N(c5ccc(-c6ccc7ccccc7c6)cc5)c5ccc(-c6cccc7ccccc67)cc5)cccc4c3c2)cc1]

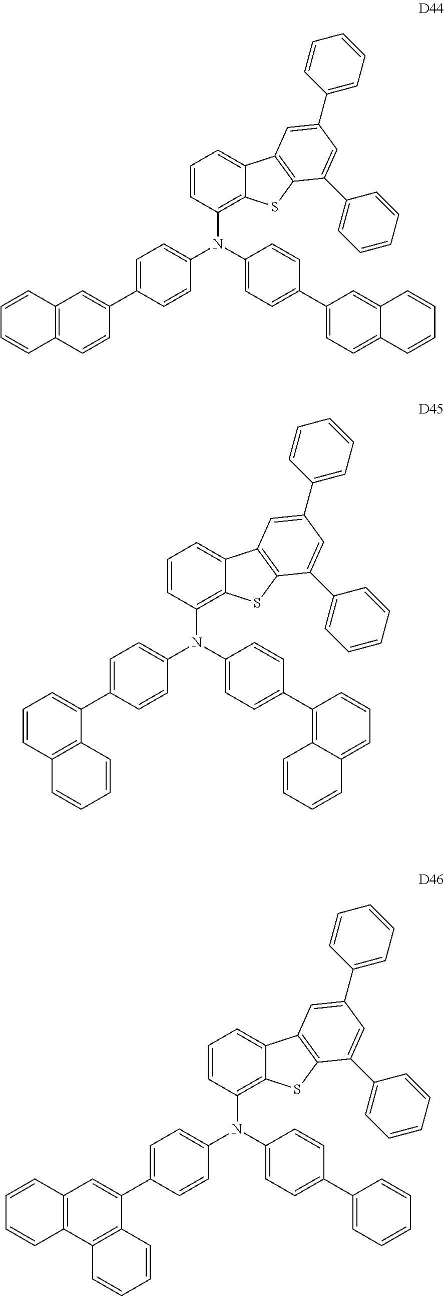 [SMILES: c1ccc(-c2cc(-c3ccccc3)c3sc4c(N(c5ccc(-c6ccc7ccccc7c6)cc5)c5ccc(-c6ccc7ccccc7c6)cc5)cccc4c3c2)cc1 ; c1ccc(-c2cc(-c3ccccc3)c3sc4c(N(c5ccc(-c6cccc7ccccc67)cc5)c5ccc(-c6cccc7ccccc67)cc5)cccc4c3c2)cc1 ; c1ccc(-c2ccc(N(c3ccc(-c4cc5ccccc5c5ccccc45)cc3)c3cccc4c3sc3c(-c5ccccc5)cc(-c5ccccc5)cc34)cc2)cc1]

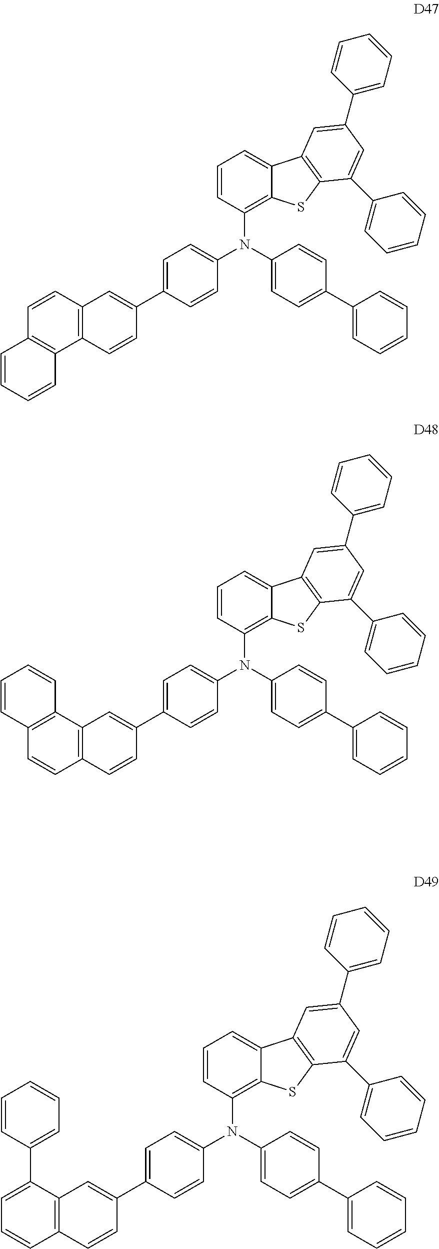 [SMILES: c1ccc(-c2ccc(N(c3ccc(-c4ccc5c(ccc6ccccc65)c4)cc3)c3cccc4c3sc3c(-c5ccccc5)cc(-c5ccccc5)cc34)cc2)cc1 ; c1ccc(-c2ccc(N(c3ccc(-c4ccc5ccc6ccccc6c5c4)cc3)c3cccc4c3sc3c(-c5ccccc5)cc(-c5ccccc5)cc34)cc2)cc1 ; c1ccc(-c2ccc(N(c3ccc(-c4ccc5cccc(-c6ccccc6)c5c4)cc3)c3cccc4c3sc3c(-c5ccccc5)cc(-c5ccccc5)cc34)cc2)cc1]

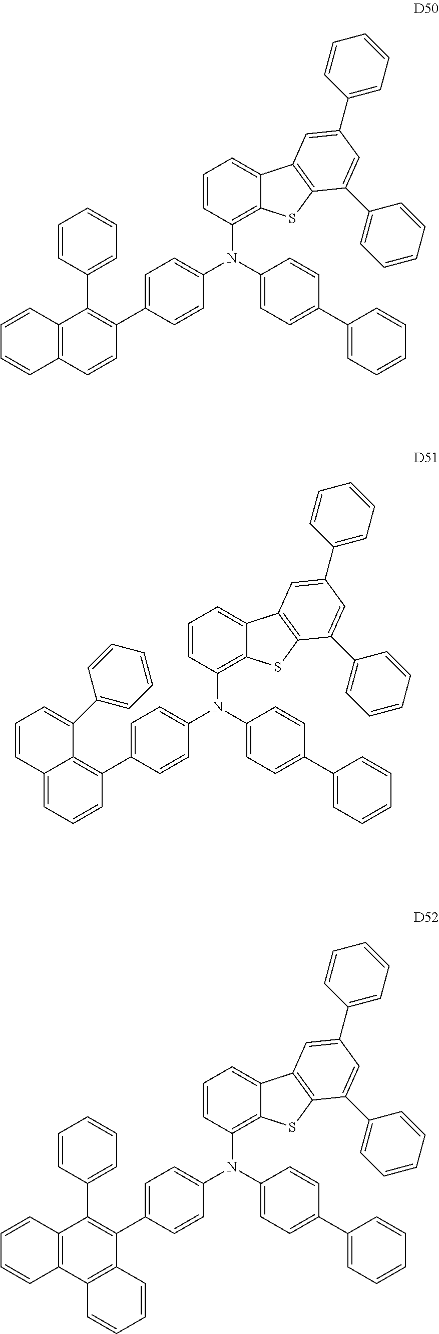 [SMILES: c1ccc(-c2ccc(N(c3ccc(-c4ccc5ccccc5c4-c4ccccc4)cc3)c3cccc4c3sc3c(-c5ccccc5)cc(-c5ccccc5)cc34)cc2)cc1 ; c1ccc(-c2ccc(N(c3ccc(-c4cccc5cccc(-c6ccccc6)c45)cc3)c3cccc4c3sc3c(-c5ccccc5)cc(-c5ccccc5)cc34)cc2)cc1 ; c1ccc(-c2ccc(N(c3ccc(-c4c(-c5ccccc5)c5ccccc5c5ccccc45)cc3)c3cccc4c3sc3c(-c5ccccc5)cc(-c5ccccc5)cc34)cc2)cc1]

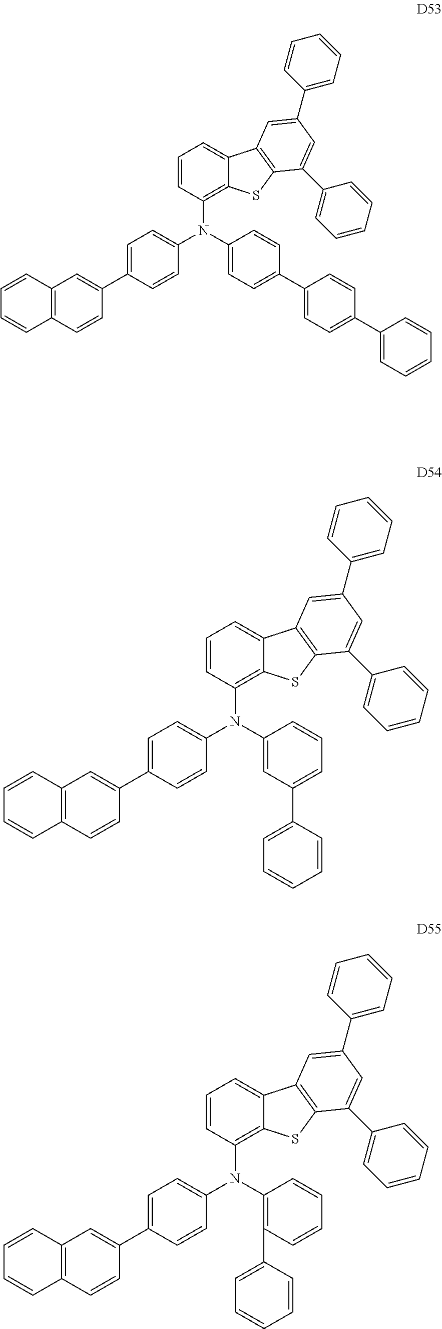 [SMILES: c1ccc(-c2ccc(-c3ccc(N(c4ccc(-c5ccc6ccccc6c5)cc4)c4cccc5c4sc4c(-c6ccccc6)cc(-c6ccccc6)cc45)cc3)cc2)cc1 ; c1ccc(-c2cccc(N(c3ccc(-c4ccc5ccccc5c4)cc3)c3cccc4c3sc3c(-c5ccccc5)cc(-c5ccccc5)cc34)c2)cc1 ; c1ccc(-c2cc(-c3ccccc3)c3sc4c(N(c5ccc(-c6ccc7ccccc7c6)cc5)c5ccccc5-c5ccccc5)cccc4c3c2)cc1]

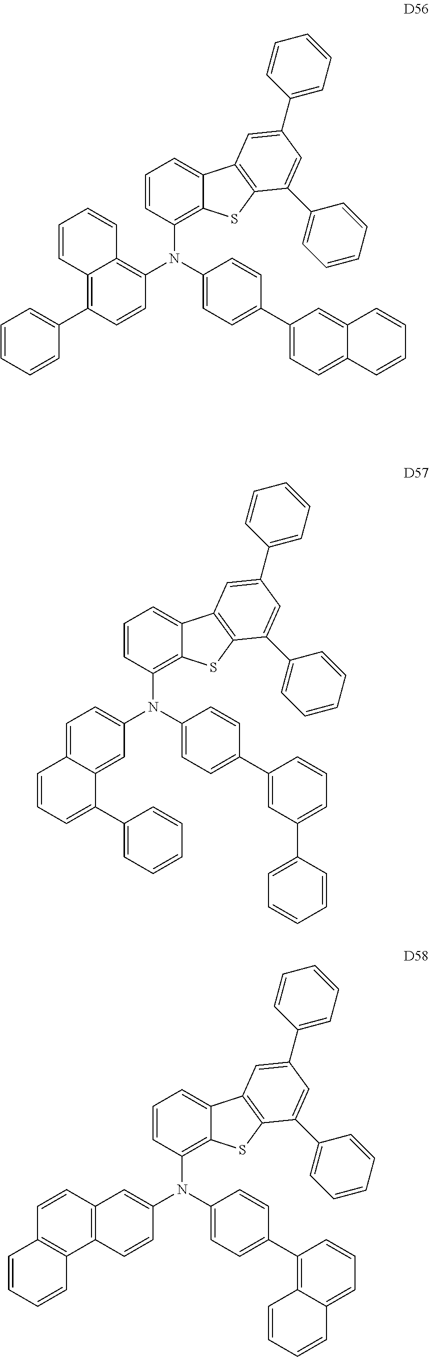

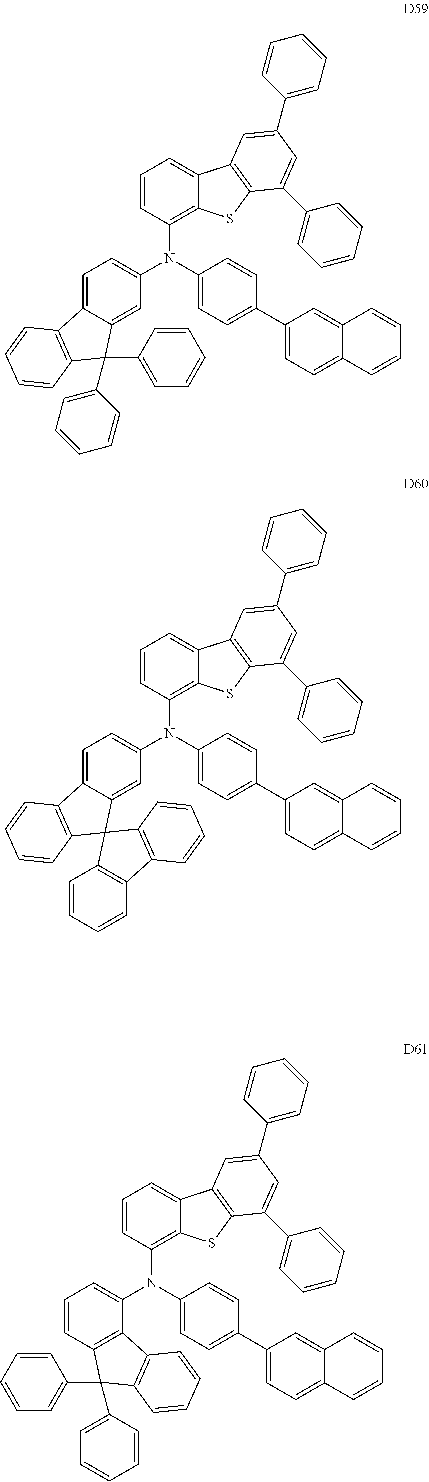 [SMILES: c1ccc(-c2cc(-c3ccccc3)c3sc4c(N(c5ccc(-c6ccc7ccccc7c6)cc5)c5ccc6c(c5)C(c5ccccc5)(c5ccccc5)c5ccccc5-6)cccc4c3c2)cc1 ; c1ccc(-c2cc(-c3ccccc3)c3sc4c(N(c5ccc(-c6ccc7ccccc7c6)cc5)c5ccc6c(c5)C5(c7ccccc7-c7ccccc75)c5ccccc5-6)cccc4c3c2)cc1 ; c1ccc(-c2cc(-c3ccccc3)c3sc4c(N(c5ccc(-c6ccc7ccccc7c6)cc5)c5cccc6c5-c5ccccc5C6(c5ccccc5)c5ccccc5)cccc4c3c2)cc1]

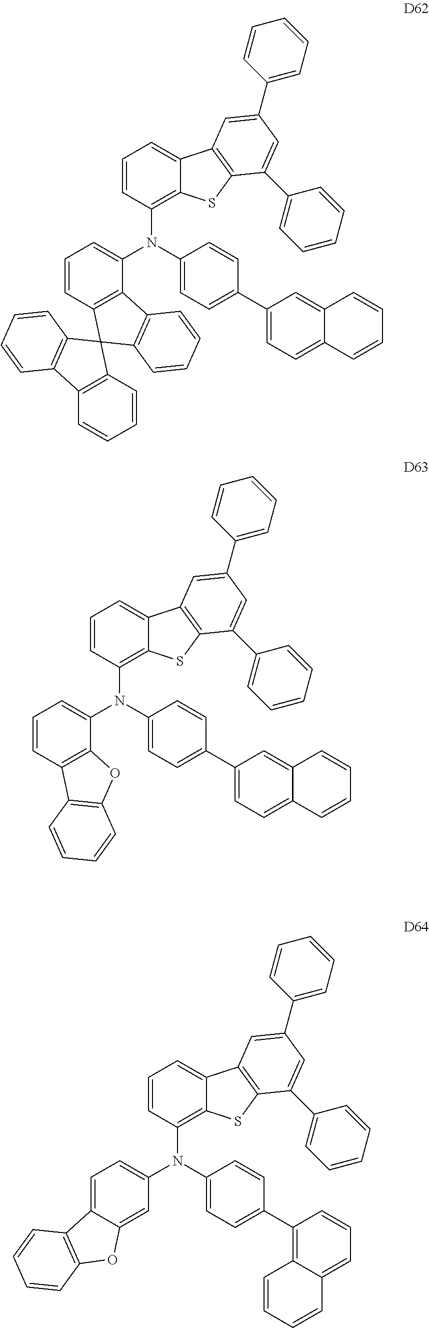 [SMILES: c1ccc(-c2cc(-c3ccccc3)c3sc4c(N(c5ccc(-c6ccc7ccccc7c6)cc5)c5cccc6c5-c5ccccc5C65c6ccccc6-c6ccccc65)cccc4c3c2)cc1 ; c1ccc(-c2cc(-c3ccccc3)c3sc4c(N(c5ccc(-c6ccc7ccccc7c6)cc5)c5cccc6c5oc5ccccc56)cccc4c3c2)cc1 ; c1ccc(-c2cc(-c3ccccc3)c3sc4c(N(c5ccc(-c6cccc7ccccc67)cc5)c5ccc6c(c5)oc5ccccc56)cccc4c3c2)cc1]

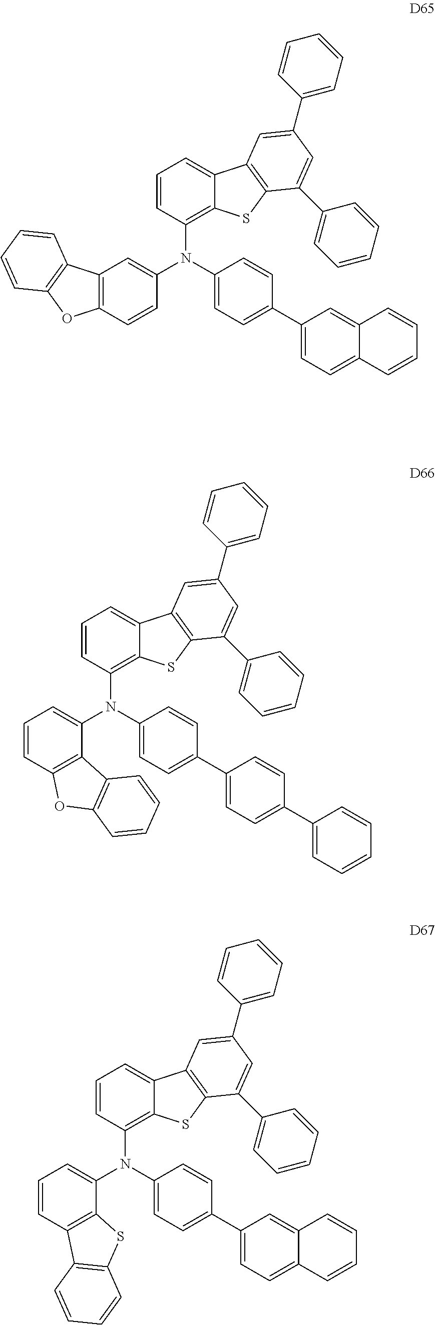 [SMILES: c1ccc(-c2cc(-c3ccccc3)c3sc4c(N(c5ccc(-c6ccc7ccccc7c6)cc5)c5ccc6oc7ccccc7c6c5)cccc4c3c2)cc1 ; c1ccc(-c2ccc(-c3ccc(N(c4cccc5c4sc4c(-c6ccccc6)cc(-c6ccccc6)cc45)c4cccc5oc6ccccc6c45)cc3)cc2)cc1 ; c1ccc(-c2cc(-c3ccccc3)c3sc4c(N(c5ccc(-c6ccc7ccccc7c6)cc5)c5cccc6c5sc5ccccc56)cccc4c3c2)cc1]

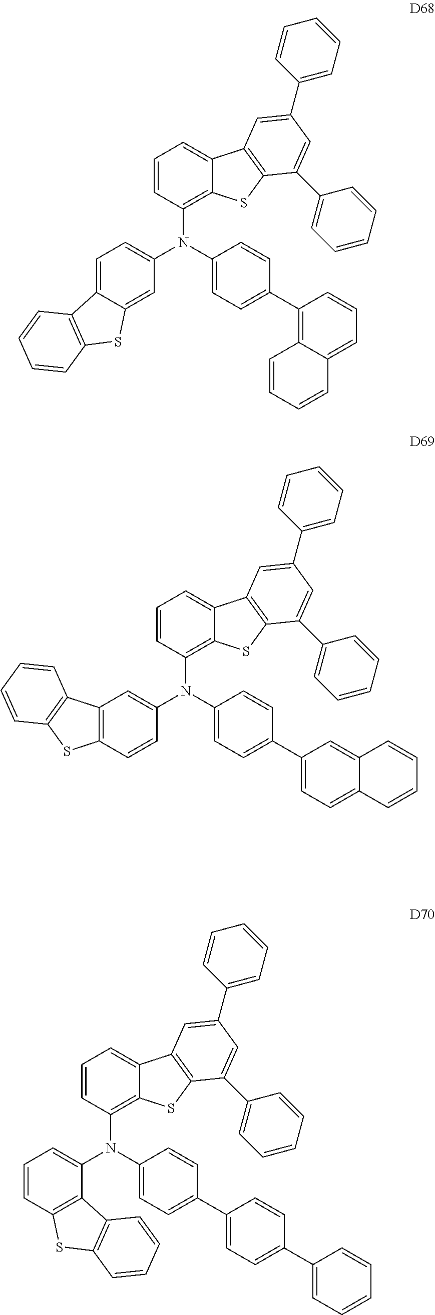 [SMILES: c1ccc(-c2cc(-c3ccccc3)c3sc4c(N(c5ccc(-c6cccc7ccccc67)cc5)c5ccc6c(c5)sc5ccccc56)cccc4c3c2)cc1 ; c1ccc(-c2cc(-c3ccccc3)c3sc4c(N(c5ccc(-c6ccc7ccccc7c6)cc5)c5ccc6sc7ccccc7c6c5)cccc4c3c2)cc1 ; c1ccc(-c2ccc(-c3ccc(N(c4cccc5c4sc4c(-c6ccccc6)cc(-c6ccccc6)cc45)c4cccc5sc6ccccc6c45)cc3)cc2)cc1]

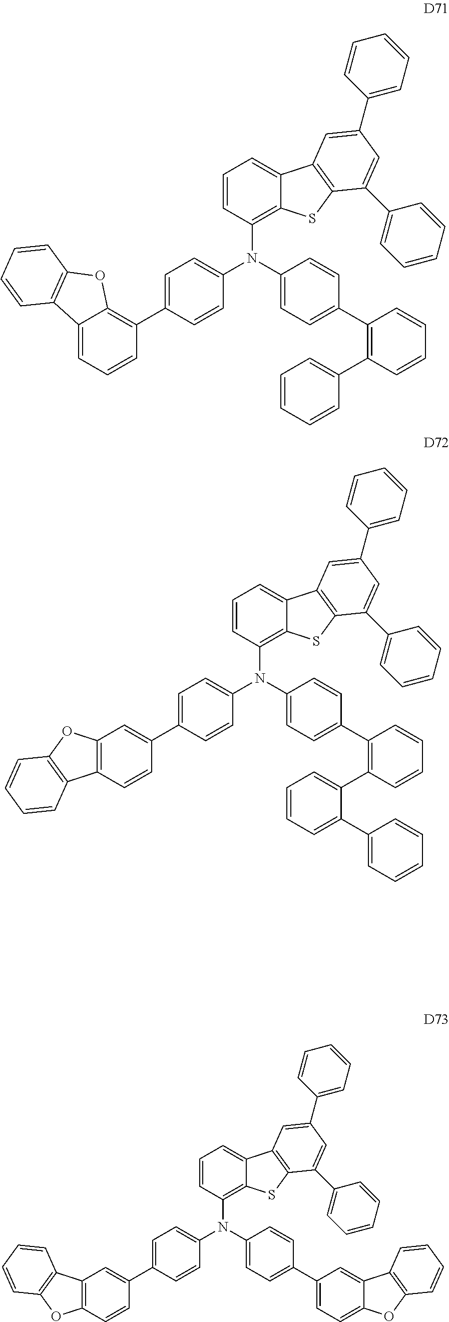 [SMILES: c1ccc(-c2cc(-c3ccccc3)c3sc4c(N(c5ccc(-c6ccccc6-c6ccccc6)cc5)c5ccc(-c6cccc7c6oc6ccccc67)cc5)cccc4c3c2)cc1 ; c1ccc(-c2cc(-c3ccccc3)c3sc4c(N(c5ccc(-c6ccc7c(c6)oc6ccccc67)cc5)c5ccc(-c6ccccc6-c6ccccc6-c6ccccc6)cc5)cccc4c3c2)cc1 ; c1ccc(-c2cc(-c3ccccc3)c3sc4c(N(c5ccc(-c6ccc7oc8ccccc8c7c6)cc5)c5ccc(-c6ccc7oc8ccccc8c7c6)cc5)cccc4c3c2)cc1]

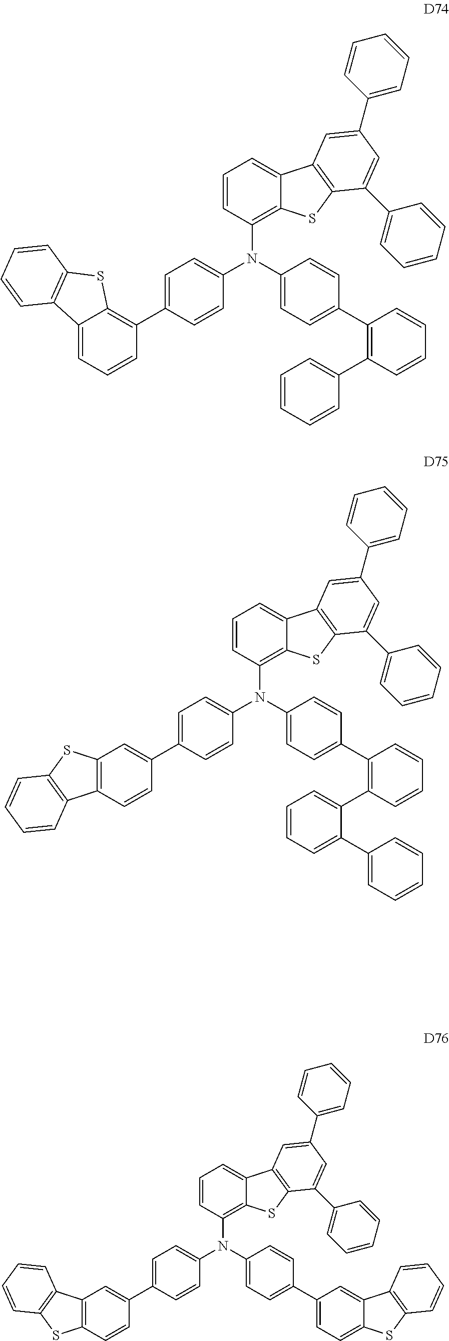 [SMILES: c1ccc(-c2cc(-c3ccccc3)c3sc4c(N(c5ccc(-c6ccccc6-c6ccccc6)cc5)c5ccc(-c6cccc7c6sc6ccccc67)cc5)cccc4c3c2)cc1 ; c1ccc(-c2cc(-c3ccccc3)c3sc4c(N(c5ccc(-c6ccc7c(c6)sc6ccccc67)cc5)c5ccc(-c6ccccc6-c6ccccc6-c6ccccc6)cc5)cccc4c3c2)cc1 ; c1ccc(-c2cc(-c3ccccc3)c3sc4c(N(c5ccc(-c6ccc7sc8ccccc8c7c6)cc5)c5ccc(-c6ccc7sc8ccccc8c7c6)cc5)cccc4c3c2)cc1]

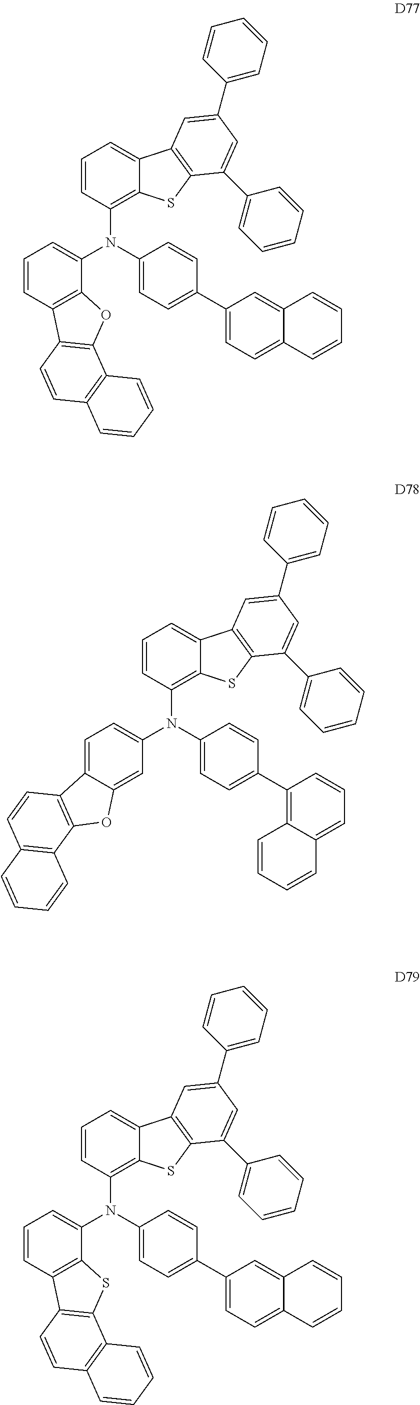 [SMILES: c1ccc(-c2cc(-c3ccccc3)c3sc4c(N(c5ccc(-c6ccc7ccccc7c6)cc5)c5cccc6c5oc5c7ccccc7ccc65)cccc4c3c2)cc1 ; c1ccc(-c2cc(-c3ccccc3)c3sc4c(N(c5ccc(-c6cccc7ccccc67)cc5)c5ccc6c(c5)oc5c7ccccc7ccc65)cccc4c3c2)cc1 ; c1ccc(-c2cc(-c3ccccc3)c3sc4c(N(c5ccc(-c6ccc7ccccc7c6)cc5)c5cccc6c5sc5c7ccccc7ccc65)cccc4c3c2)cc1]

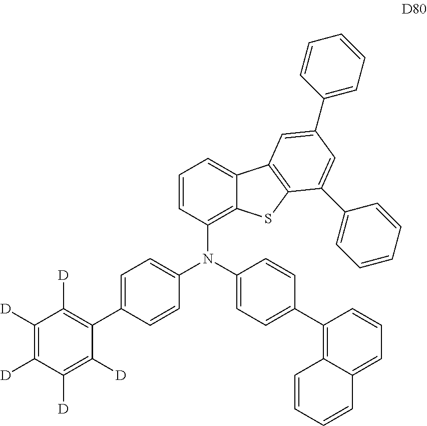 [SMILES: [2H]c1c([2H])c([2H])c(-c2ccc(N(c3ccc(-c4cccc5ccccc45)cc3)c3cccc4c3sc3c(-c5ccccc5)cc(-c5ccccc5)cc34)cc2)c([2H])c1[2H]]

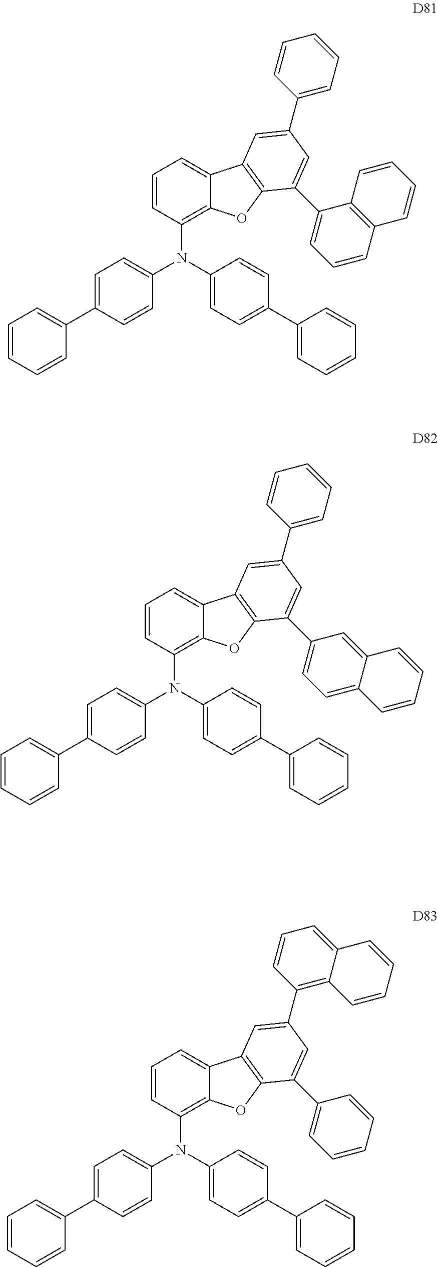 [SMILES: c1ccc(-c2ccc(N(c3ccc(-c4ccccc4)cc3)c3cccc4c3oc3c(-c5cccc6ccccc56)cc(-c5ccccc5)cc34)cc2)cc1 ; c1ccc(-c2ccc(N(c3ccc(-c4ccccc4)cc3)c3cccc4c3oc3c(-c5ccc6ccccc6c5)cc(-c5ccccc5)cc34)cc2)cc1 ; c1ccc(-c2ccc(N(c3ccc(-c4ccccc4)cc3)c3cccc4c3oc3c(-c5ccccc5)cc(-c5cccc6ccccc56)cc34)cc2)cc1]

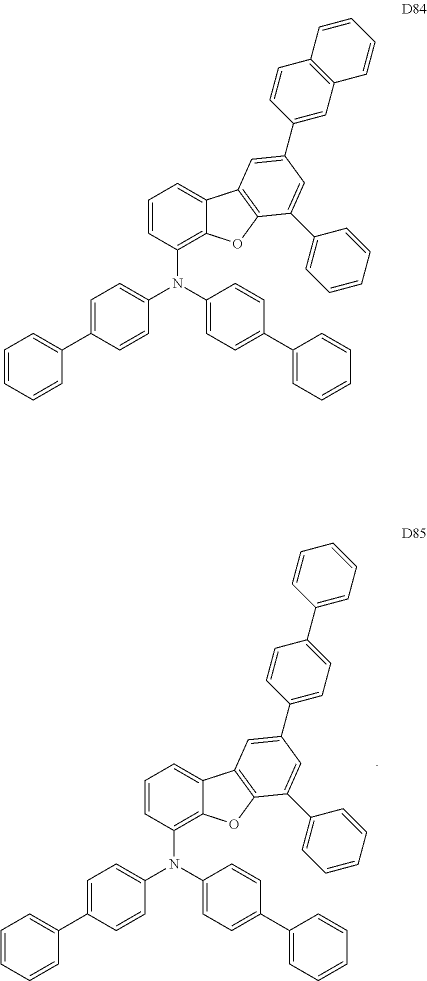 [SMILES: c1ccc(-c2ccc(N(c3ccc(-c4ccccc4)cc3)c3cccc4c3oc3c(-c5ccccc5)cc(-c5ccc6ccccc6c5)cc34)cc2)cc1 ; c1ccc(-c2ccc(-c3cc(-c4ccccc4)c4oc5c(N(c6ccc(-c7ccccc7)cc6)c6ccc(-c7ccccc7)cc6)cccc5c4c3)cc2)cc1]

The amine compound of an embodiment, represented by Formula 1 may include at least one dibenzoheterole skeleton substituted with two aryl groups (for example, phenyl groups). In the amine compound of an embodiment, one aryl group may be substituted at position 6 of a dibenzoheterole skeleton

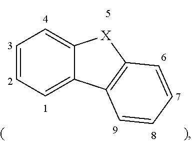

and the remaining aryl group may be substituted at position 1, 2, 8 or 9 of the dibenzoheterole skeleton. Here, X of the dibenzoheterole skeleton may be O or S.

In the amine compound of an embodiment, the aryl group substituted at position 6 of the dibenzoheterole skeleton may be oriented as if to cover (e.g., close to) the heteroatom (O or S) of the dibenzoheterole skeleton, to contribute to the stability of a radical or radical cation state. In addition, in the amine compound of an embodiment, the aryl group substituted at position 1, 2, 8 or 9 of the dibenzoheterole skeleton may be oriented as if to spread out (e.g., to extend the size) of a molecule to increase intermolecular interaction to improve hole transport capacity, and may contribute to the reduction of a driving voltage and the increase of efficiency. Accordingly, when the amine compound of an embodiment is utilized as a material of a light emitting element, the efficiency and life characteristics of the light emitting element may be improved.

In some embodiments, when the light emitting element ED of an embodiment includes multiple hole transport layers, a hole transport layer adjacent to the emission layer selected from among the multiple hole transport layers may include the amine compound of an embodiment.

In some embodiments, the light emitting element ED of an embodiment may further include a material of a hole transport region explained below, in addition to the amine compound of an embodiment in the hole transport region HTR.

The hole transport region HTR may include a compound represented by Formula H-1.

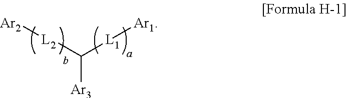

In Formula H-1 above, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. “a” and “b” may each independently be an integer of 0 to 10. In some embodiments, when “a” or “b” is an integer of 2 or more, two or more L₁ and L₂ may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound (e.g., a compound including a single amine group). In some embodiments, the compound represented by Formula H-1 may be a diamine compound in which at least one selected from among Ar₁ to Ar₃ includes an amine group as a substituent. In some embodiments, the compound represented by Formula H-1 may be a carbazole-based compound in which at least one selected from among Ar₁ and Ar₂ includes a substituted or unsubstituted carbazole group, or a fluorene-based compound in which at least one selected from among Ar₁ and Ar₂ includes a substituted or unsubstituted fluorene group.

The compound represented by Formula H-1 may be represented by any one selected from among the compounds in Compound Group H. However, the compounds listed in Compound Group H are only examples, and the compound represented by Formula H-1 is not limited to the compounds represented in Compound Group H.

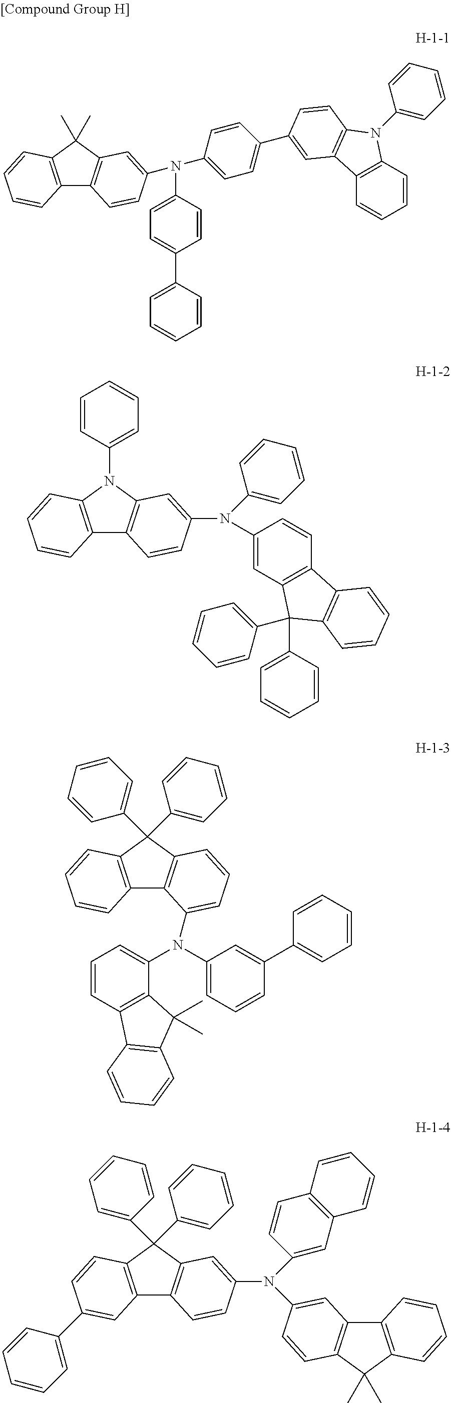 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

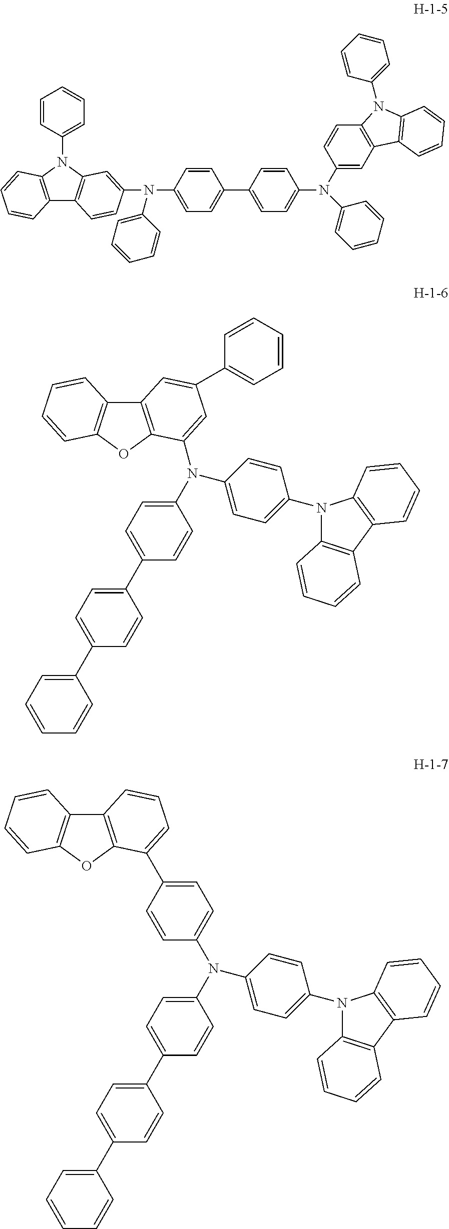 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

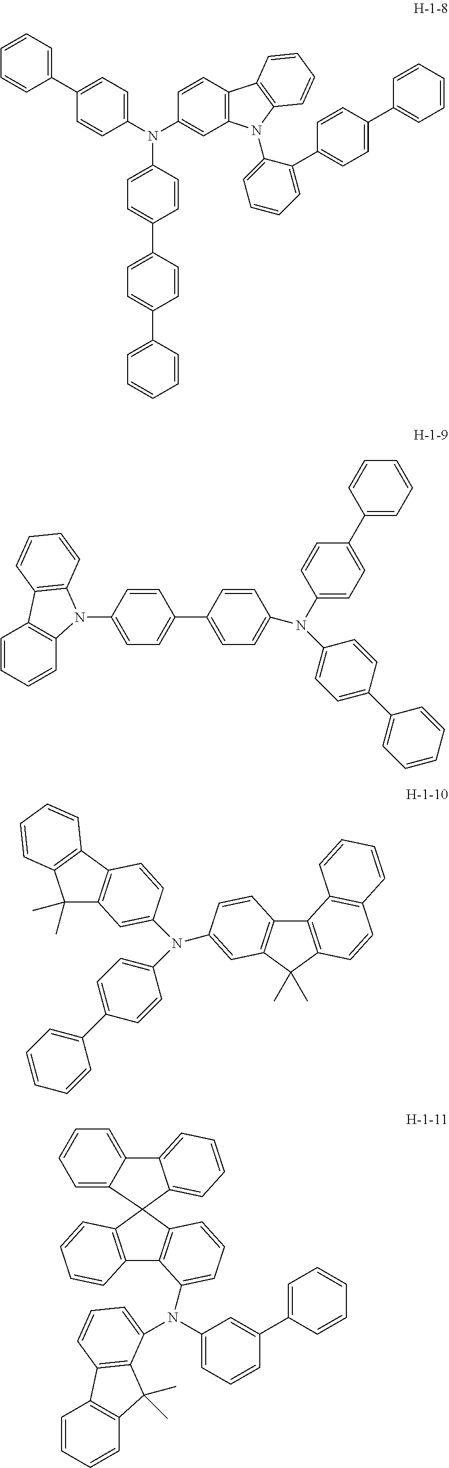

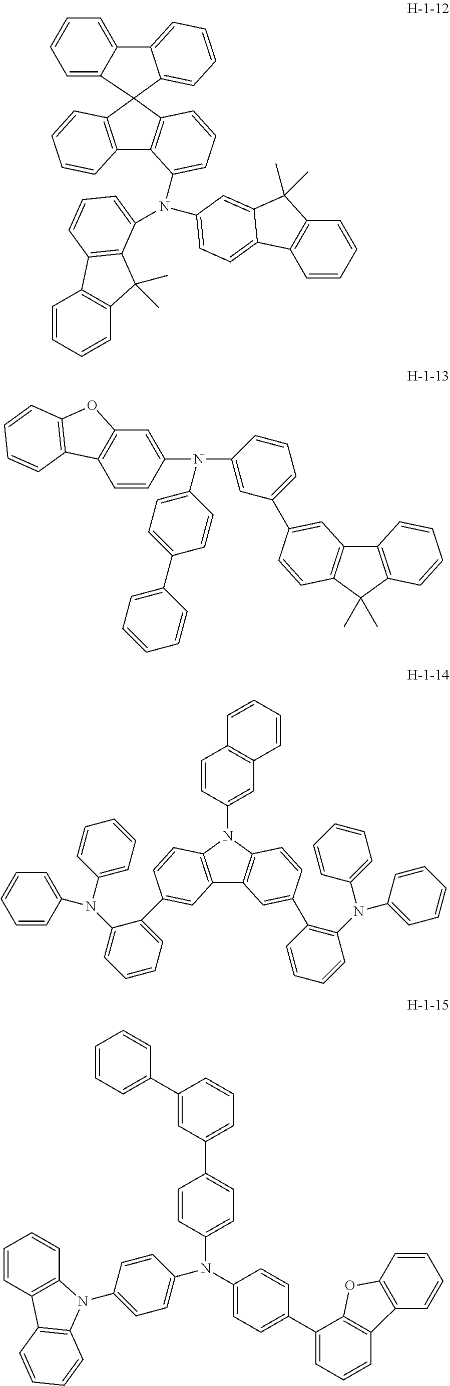 [SMILES: CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

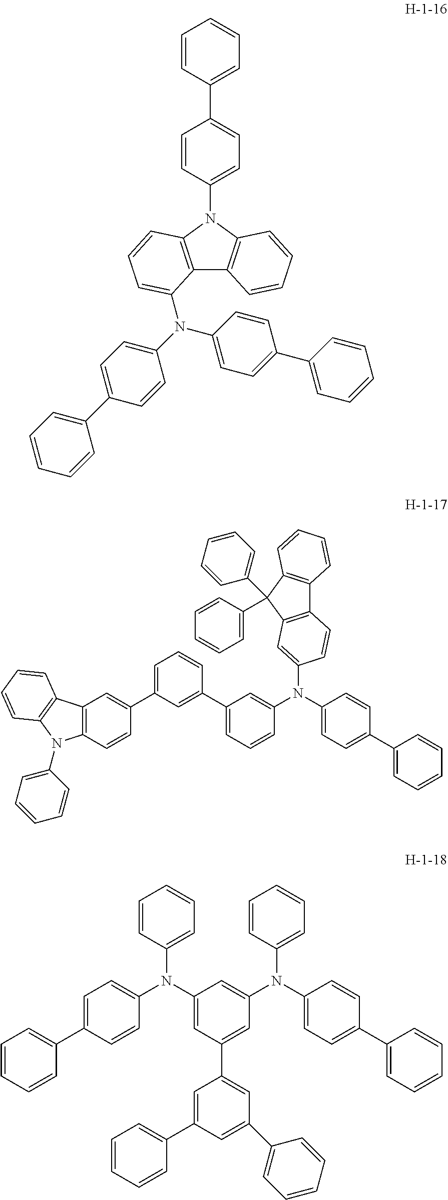 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

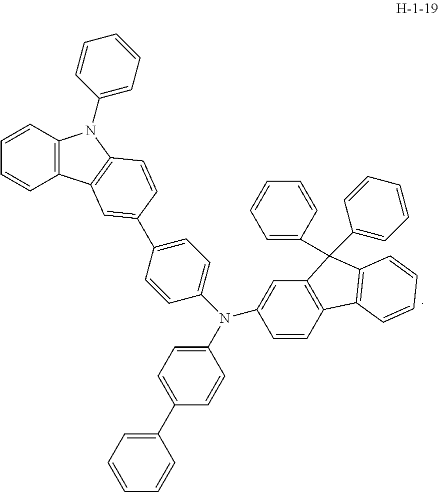 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], and dipyrazino[2,3-f:2′,3′-h] quinoxaline-2,3,6,7,10,11-hexacarbonitrile (HAT-CN).

The hole transport region HTR may include one or more carbazole derivatives such as N-phenyl carbazole and/or polyvinyl carbazole, one or more fluorene-based derivatives, N,N′-bis(3-methylphenyl)-N,N′-diphenyl-[1,1′-biphenyl]-4,4′-diamine (TPD), one or more triphenylamine-based derivatives such as 4,4′,4″-tris(N-carbazolyl)triphenylamine (TCTA), N,N′-di(1-naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above described compounds of the hole transport region in at least one selected from among the hole injection layer HIL, hole transport layer HTL, and electron blocking layer EBL.

The thickness of the hole transport region HTR may be from about 100 Å to about 10,000 Å, for example, from about 100 Å to about 5,000 Å. When the hole transport region HTR includes a hole injection layer HIL, the thickness of the hole injection region HIL may be, for example, from about 30 Å to about 1,000 Å. When the hole transport region HTR includes a hole transport layer HTL, the thickness of the hole transport layer HTL may be from about 30 Å to about 1,000 Å. For example, when the hole transport region HTR includes an electron blocking layer EBL, the thickness of the electron blocking layer EBL may be from about 10 Å to about 1,000 Å. When the thicknesses of the hole transport region HTR, the hole injection layer HIL, the hole transport layer HTL and the electron blocking layer EBL satisfy the above-described ranges, satisfactory hole transport properties may be achieved without a substantial increase of a driving voltage.

The hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may include at least one of a metal halide compound, a quinone derivative, a metal oxide, and a cyano group-containing compound, but the present disclosure is not limited thereto. For example, the p-dopant may include one or more metal halide compounds such as CuI and/or RbI, quinone derivatives such as tetracyanoquinodimethane (TCNQ) and/or 2,3,5,6-tetrafluoro-7,7,8,8-tetracyanoquinodimethane (F4-TCNQ), metal oxides such as tungsten oxide and/or molybdenum oxide, cyano group-containing compounds such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but the present disclosure is not limited thereto.

As described above, the hole transport region HTR may further include at least one selected from among a buffer layer and an electron blocking layer EBL in addition to the hole injection layer HIL and the hole transport layer HTL. The buffer layer may compensate for a resonance distance according to the wavelength of light emitted from an emission layer EML and may thus increase emission efficiency. Materials which may be included in the hole transport region HTR may be utilized as a material to be included in the buffer layer. The electron blocking layer EBL is a layer that serves the role of blocking or substantially blocking the injection of electrons from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed utilizing a single material, a single layer formed utilizing a plurality of different materials, or a multilayer structure having a plurality of layers formed utilizing a plurality of different materials.

In the light emitting element ED of an embodiment, the emission layer EML may be to emit light selected from among red light, green light, blue light, white light and cyan light. The light emitting element ED of an embodiment may include the amine compound of an embodiment in a hole transport region HTR and may show high efficiency and excellent or suitable life characteristics in an emission region which emits the light. However, the present disclosure is not limited thereto.

In the light emitting element ED of an embodiment, the emission layer EML may include one or more anthracene derivatives, pyrene derivatives, fluoranthene derivatives, chrysene derivatives, dihydrobenzanthracene derivatives, and/or triphenylene derivatives. For example, the emission layer EML may include one or more anthracene derivatives and/or pyrene derivatives.

In the light emitting elements ED of embodiments, shown in FIG. 3 to FIG. 6, the emission layer EML may include a host and a dopant, and the emission layer EML may include a compound represented by Formula E-1. The compound represented by Formula E-1 may be utilized as a fluorescence host material.

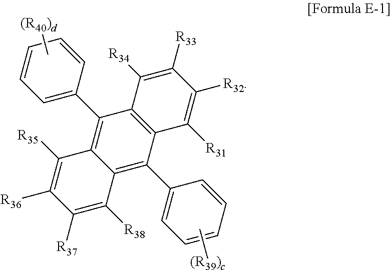

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

Formula E-1 may be represented by any one selected from among Compound E1 to Compound E19.

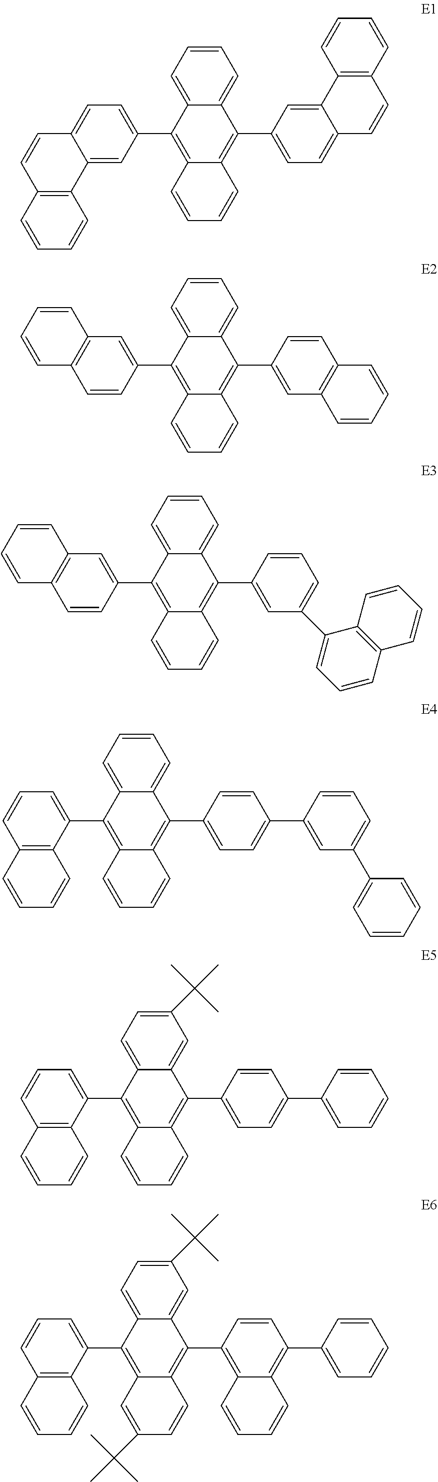

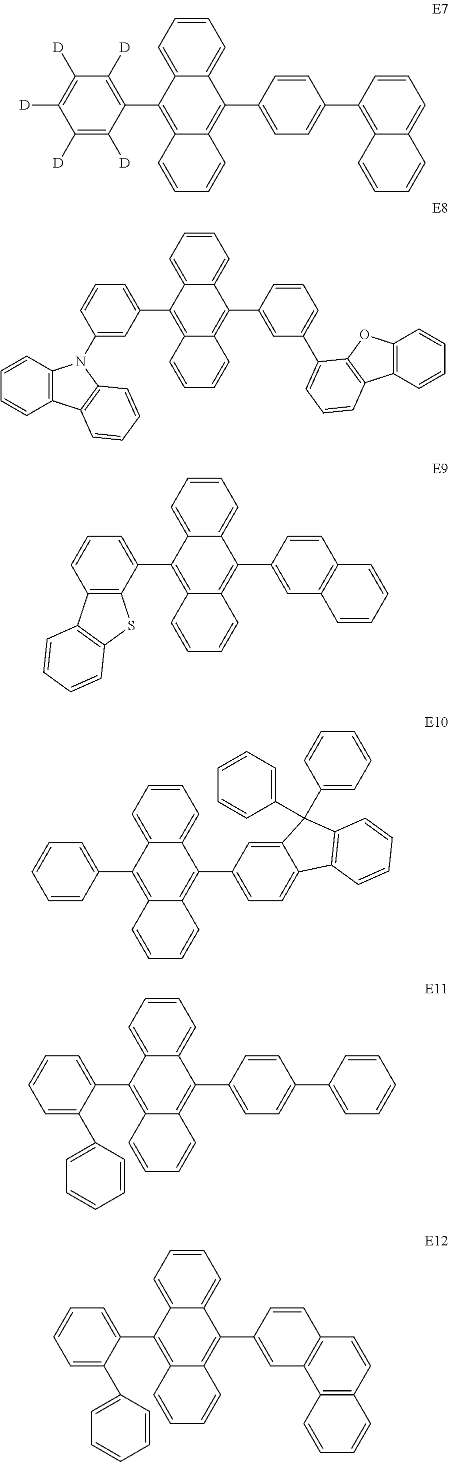

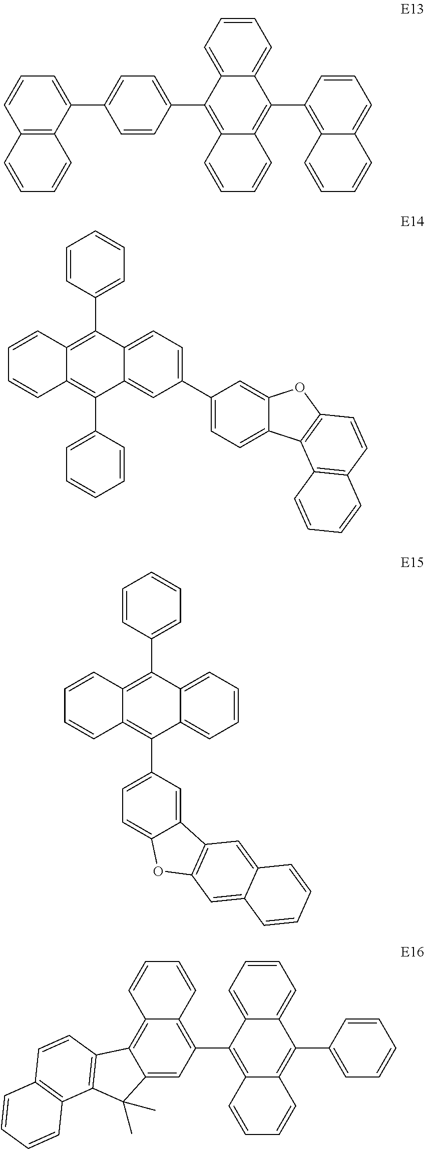

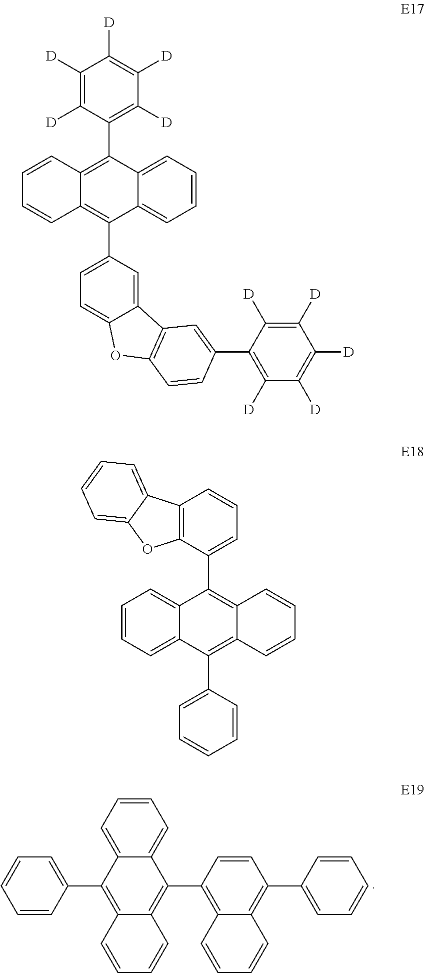

In an embodiment, the emission layer EML may include a compound represented by Formula E-2a or Formula E-2b. The compound represented by Formula E-2a or Formula E-2b may be utilized as a phosphorescence host material.

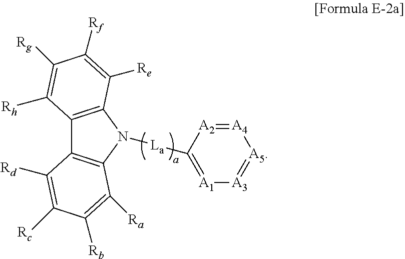

In Formula E-2a, “a” may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when “a” is an integer of 2 or more, a plurality of La's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be combined with an adjacent group to form a ring. Rₐ to Rᵢ may be combined with an adjacent group to form a hydrocarbon ring or a heterocycle including N, O, S, etc. as a ring-forming atom.

In some embodiments, in Formula E-2a, two or three selected from A₁ to A₅ may be N, and the remainder (e.g., the rest) may be CRᵢ.

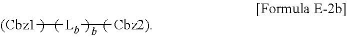

In Formula E-2b, Cbz1 and Cbz2 may each independently be an unsubstituted carbazole group, or a carbazole group substituted with an aryl group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms. “b” is an integer of 0 to 10, and when “b” is an integer of 2 or more, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among the compounds in Compound Group E-2. However, the compounds listed in Compound Group E-2 are only examples, and the compound represented by Formula E-2a or Formula E-2b is not limited to the compounds represented in Compound Group E-2.

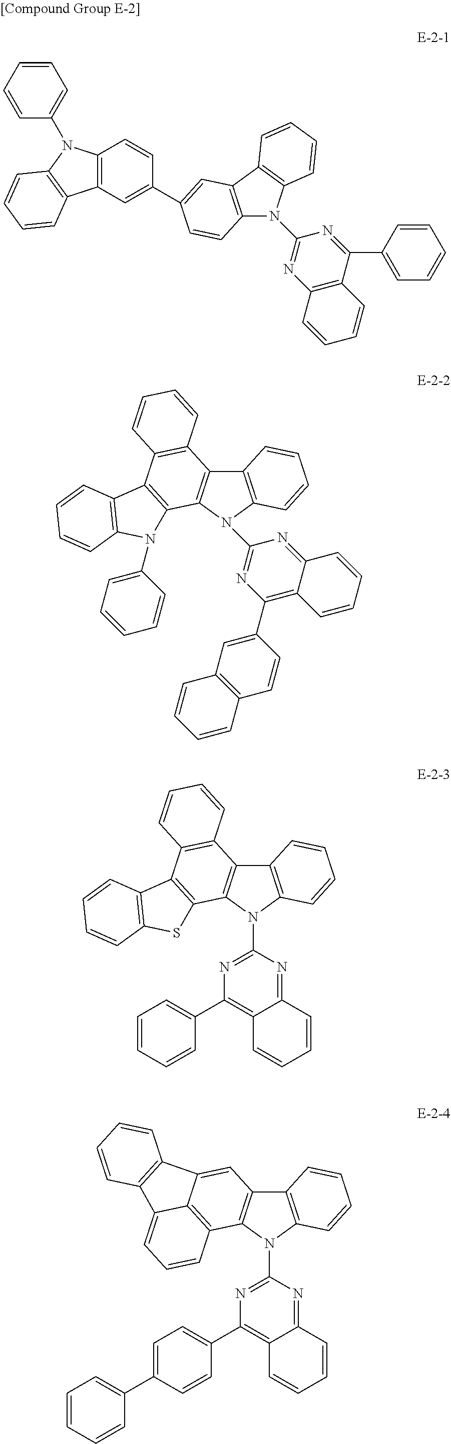

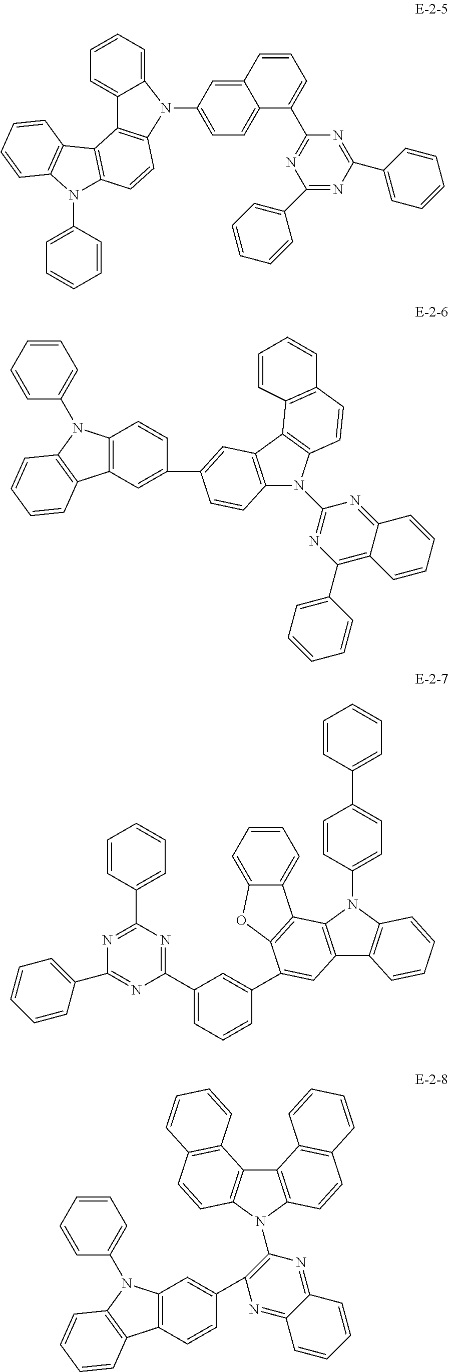 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

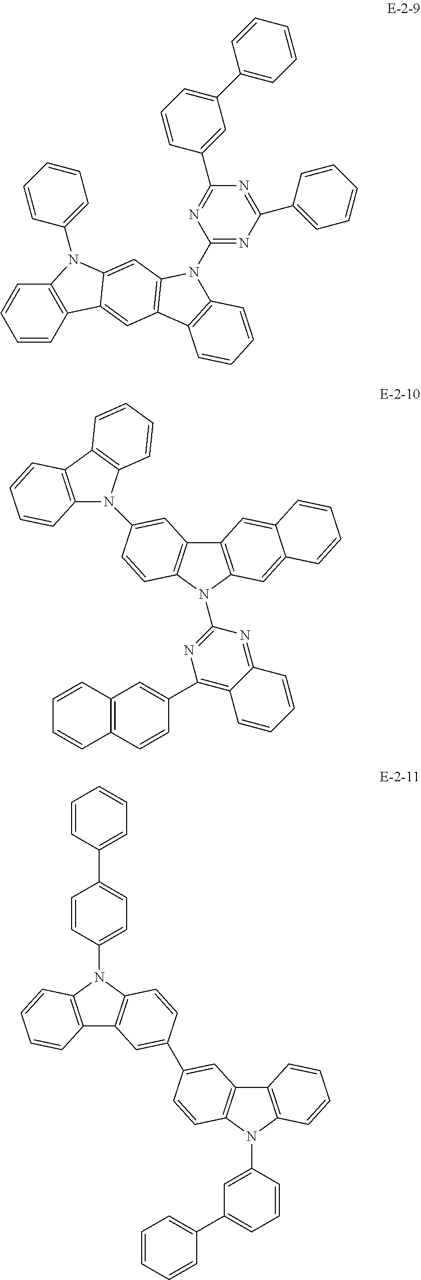

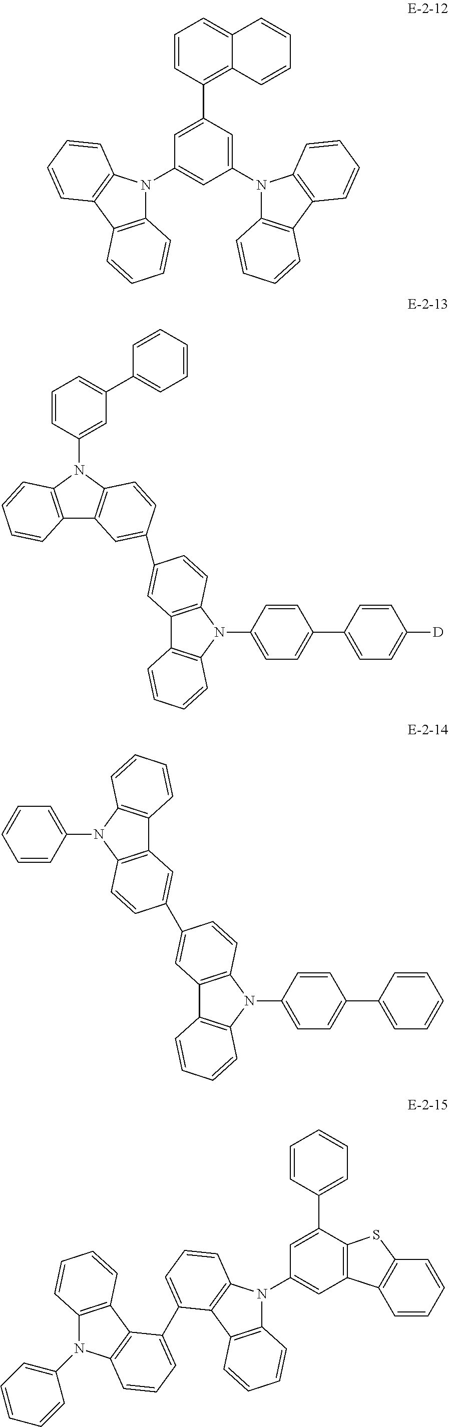 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

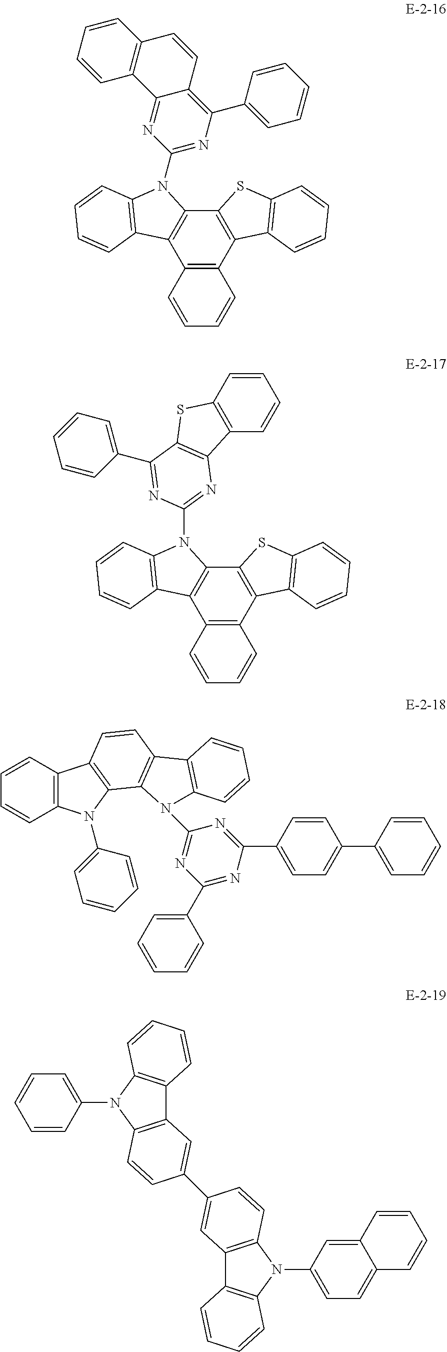 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

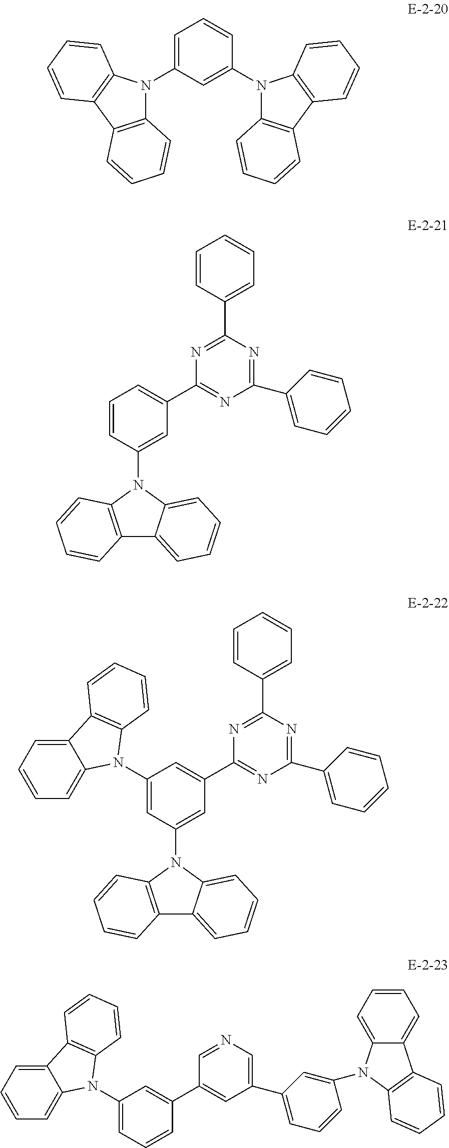 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

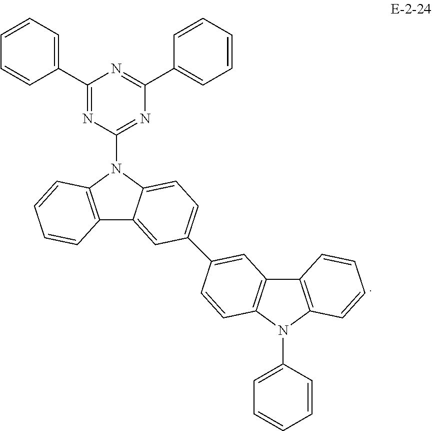 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material in the art suitable as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the present disclosure is not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be utilized as the host material.

The emission layer EML may include a compound represented by Formula M-a or Formula M-b. The compound represented by Formula M-a or Formula M-b may be utilized as a phosphorescence dopant material. In some embodiments, the compound represented by Formula M-a or Formula M-b may be utilized as an auxiliary dopant material.

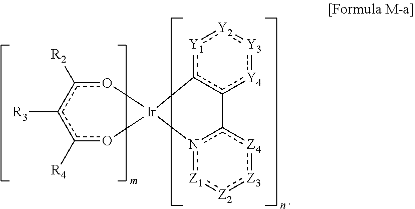

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be combined with an adjacent group to form a ring. In Formula M-a, “m” is 0 or 1, and “n” is 2 or 3. In Formula M-a, when “m” is 0, “n” is 3, and when “m” is 1, “n” is 2.

The compound represented by Formula M-a may be utilized as a phosphorescence dopant.

The compound represented by Formula M-a may be represented by any one selected from among Compounds M-a1 to M-a25. However, Compounds M-a1 to M-a25 are examples, and the compound represented by Formula M-a is not limited to the compounds represented by Compounds M-a1 to M-a25.

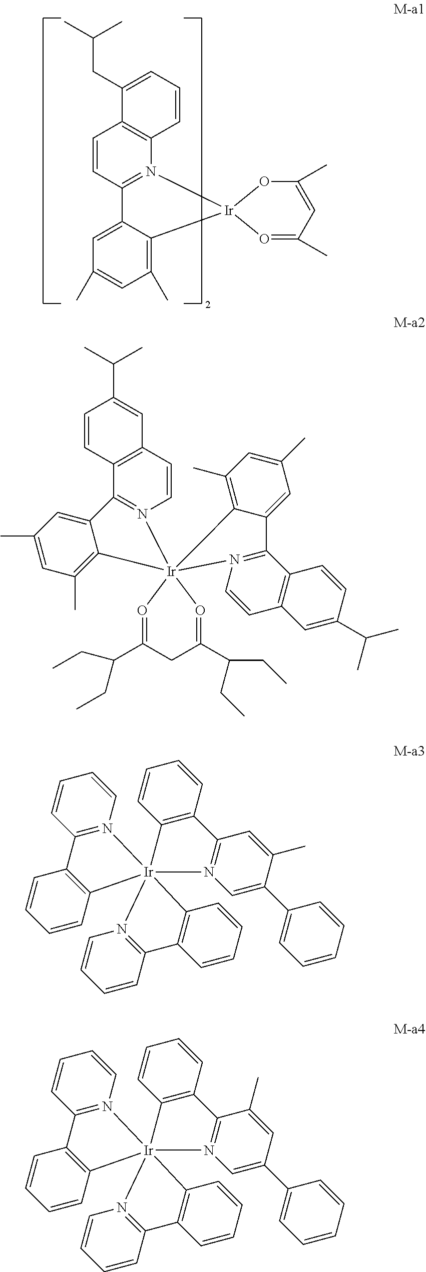

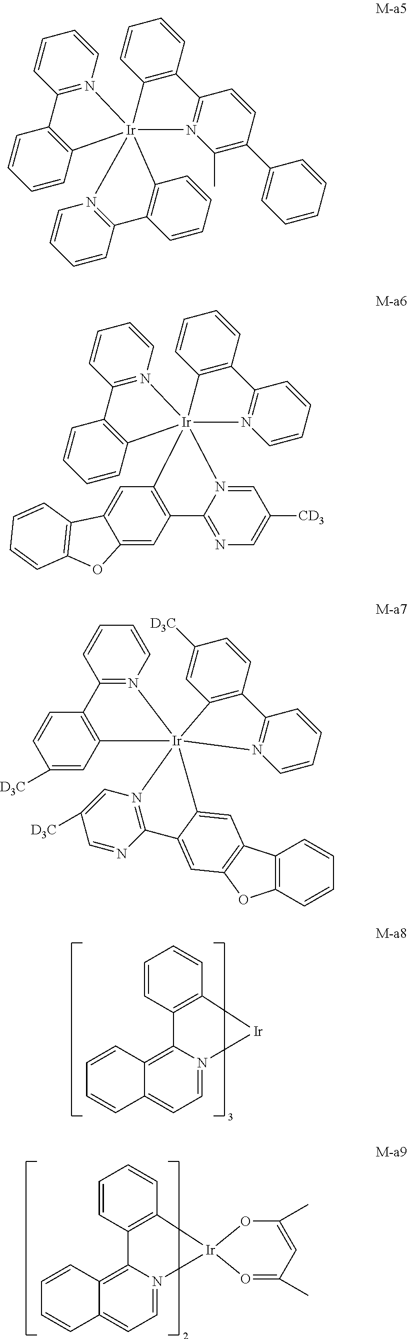

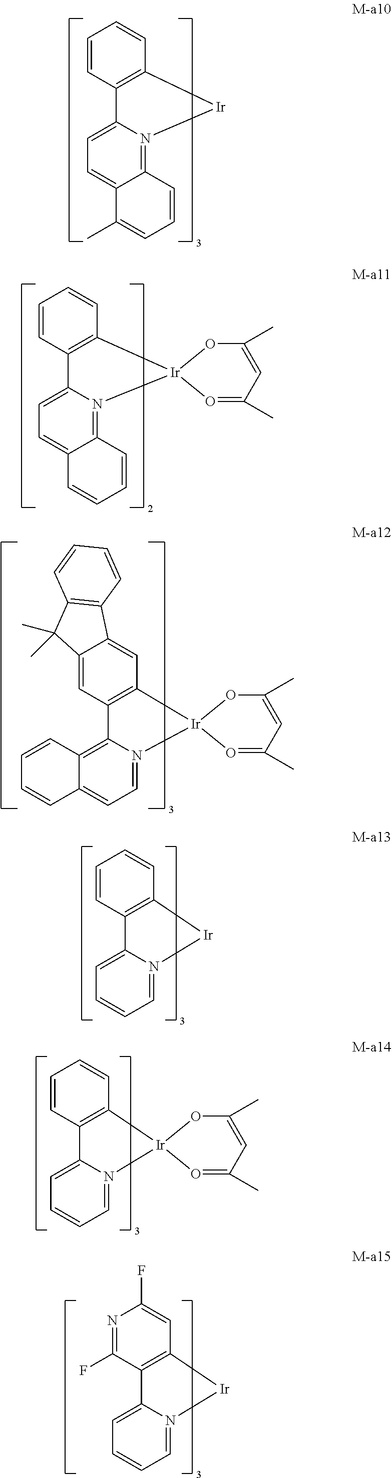

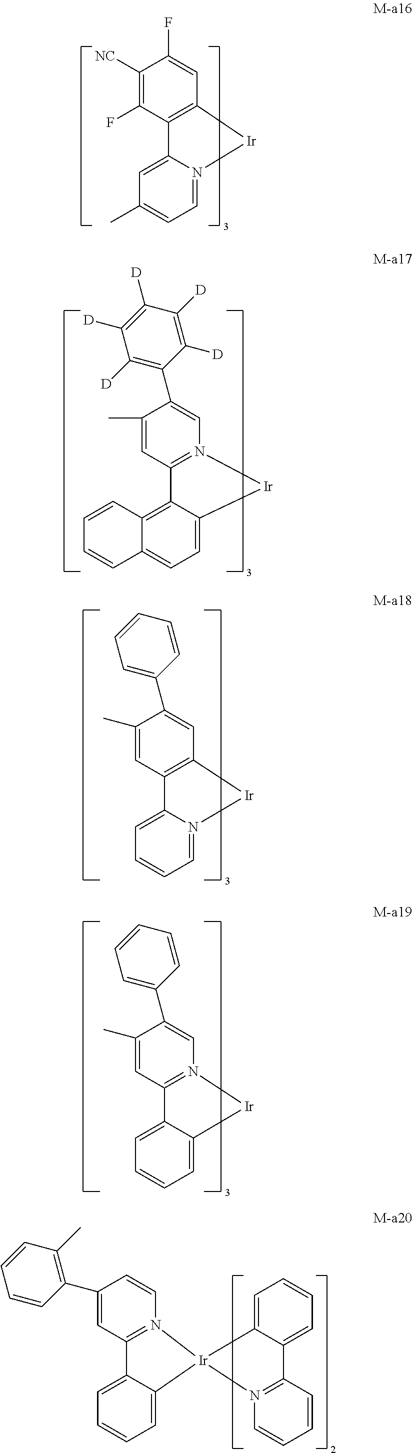

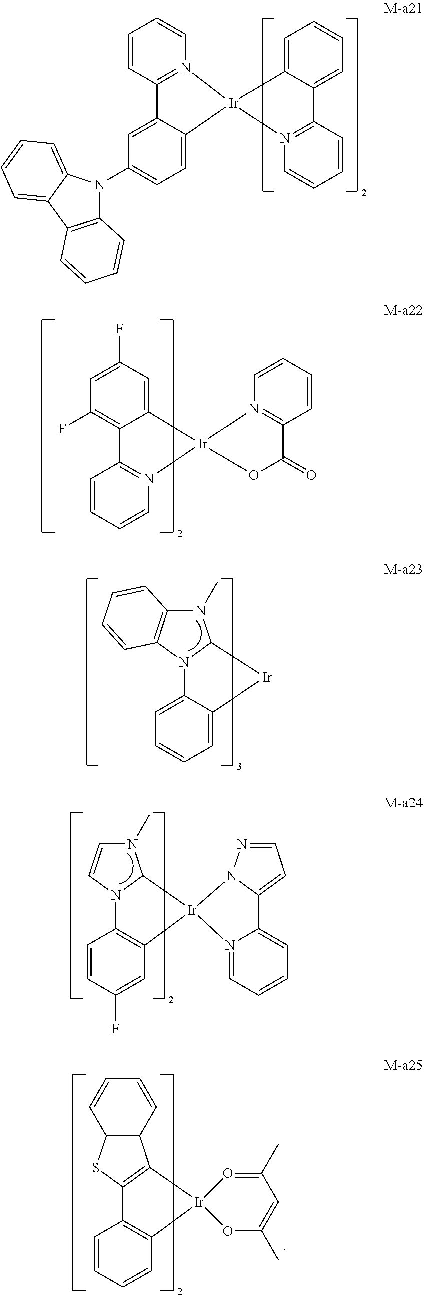

Compound M-a1 and Compound M-a2 may be utilized as red dopant materials, and Compound M-a3 to Compound M-a7 may be utilized as green dopant materials.

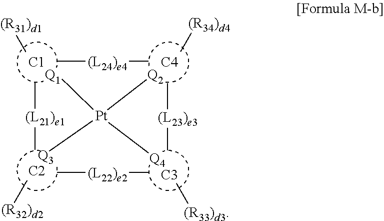

In Formula M-b, Q₁ to Q₄ may each independently be C or N, and C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

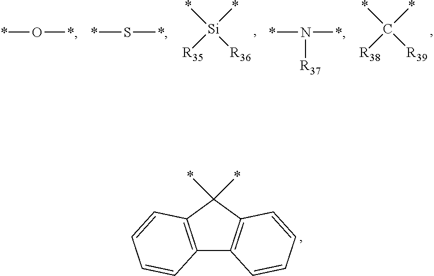

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or combined with an adjacent group to form a ring, and d1 to d4 may each independently be an integer of 0 to 4.

The compound represented by Formula M-b may be utilized as a blue phosphorescence dopant or a green phosphorescence dopant. In some embodiments, the compound represented by Formula M-b may be an auxiliary dopant and may be further included in the emission layer EML.

The compound represented by Formula M-b may be represented by any one selected from among the compounds below. However, the compounds below are examples, and the compound represented by Formula M-b is not limited to the compounds represented below.

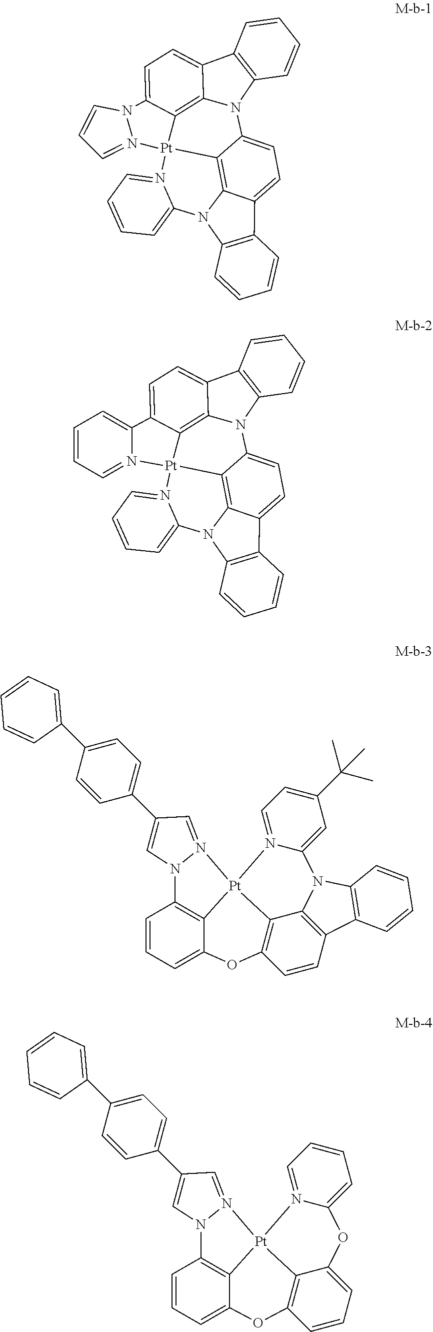

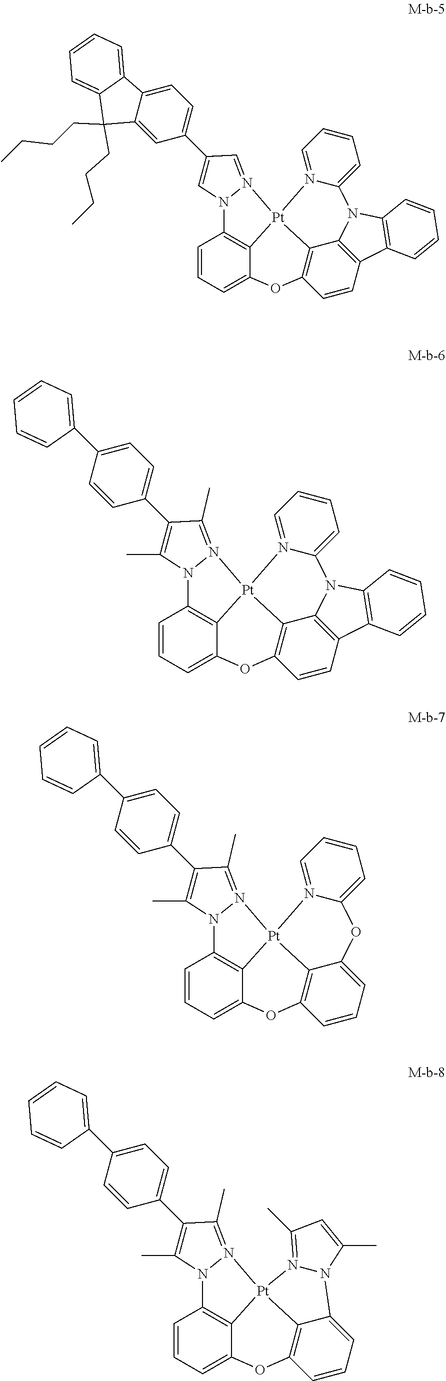

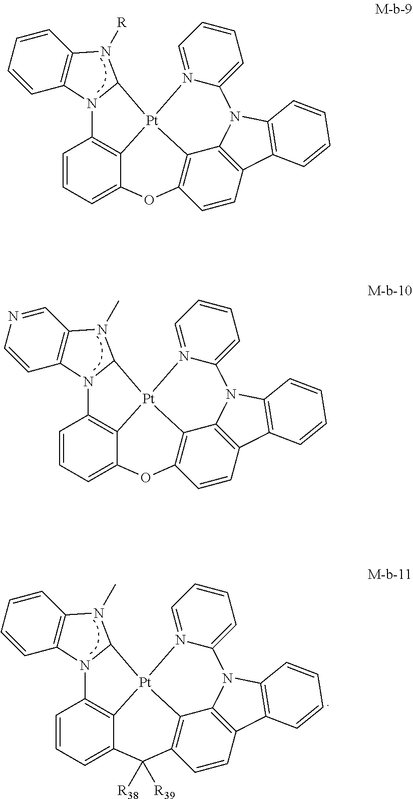

In the compounds above, R, R₃₈, and R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may include a compound represented by any one selected from among Formula F-a to Formula F-c. The compounds represented by Formula F-a to Formula F-c may be utilized as fluorescence dopant materials.

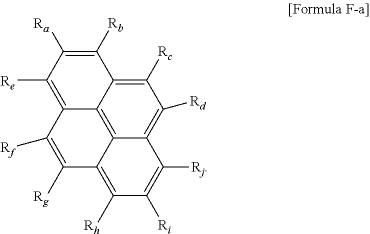

In Formula F-a, two groups selected from Rₐ to R; may each independently be substituted with *—NAr₁Ar₂. The remainder groups not substituted with *—NAr₁Ar₂ selected from among Rₐ to R; may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one selected from among Ar₁ and Ar₂ may be a heteroaryl group including O or S as a ring-forming atom.

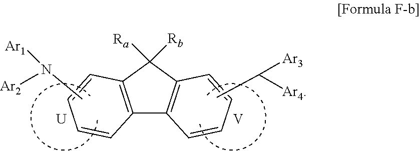

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, and/or may be combined with an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, when the number of U or V is 1, one ring indicated by U or V forms a fused ring at the designated part (e.g., at the part indicated by U or V), and when the number of U or V is 0, a ring indicated by U or V does not exist. For example, when the number of U is 0, and the number of V is 1, or when the number of U is 1, and the number of V is 0, a fused ring having the fluorene core of Formula F-b may be a ring (e.g., cyclic) compound with four rings. In some embodiments, when the number of both (e.g., simultaneously) U and V is 0, the fused ring of Formula F-b may be a ring compound with three rings. In some embodiments, when the number of both (e.g., simultaneously) U and V is 1, a fused ring having the fluorene core of Formula F-b may be a ring compound with five rings.

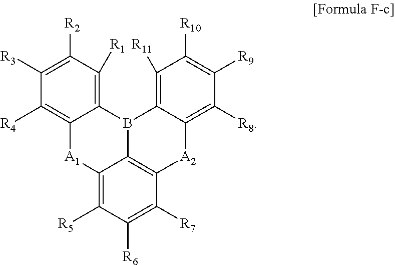

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or combined with an adjacent group to form a ring.

In Formula F-c, A₁ and A₂ may each independently be combined with the substituents of an adjacent ring to form a fused ring. For example, when A₁ and A₂ may each independently be NRₘ, A₁ may be combined with R₄ or R₅ to form a ring. In some embodiments, A₂ may be combined with R₇ or R₈ to form a ring.

In an embodiment, the emission layer EML may include as a suitable dopant material, a styryl derivative (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and/or 4,4′-bis[2-(4-(N,N-diphenylamino)phenyl)vinyl]biphenyl (DPAVBi)), perylene and a derivative thereof (for example, 2,5,8,11-tetra-t-butylperylene (TBP)), pyrene and a derivative thereof (for example, 1,1-dipyrene, 1,4-dipyrenylbenzene, and/or 1,4-bis(N,N-diphenylamino)pyrene), etc.

In an embodiment, when multiple emission layers EML are included, at least one emission layer EML may include a suitable phosphorescence dopant material. For example, the phosphorescence dopant may utilize a metal complex including iridium (Ir), platinum (Pt), osmium (Os), gold (Au), titanium (Ti), zirconium (Zr), hafnium (Hf), europium (Eu), terbium (Tb) and/or thulium (Tm). For example, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir₆), and/or platinum octaethyl porphyrin (PtOEP) may be utilized as the phosphorescence dopant. However, the present disclosure is not limited thereto.

In some embodiments, the emission layer EML may include a hole transport host and an electron transport host. In some embodiments, the emission layer EML may include an auxiliary dopant and a light emitting dopant. In some embodiments, the auxiliary dopant may include a phosphorescence dopant material or a thermally activated delayed fluorescence dopant. For example, in an embodiment, the emission layer EML may include a hole transport host, an electron transport host, an auxiliary dopant, and a light emitting dopant.

In some embodiments, exiplex may be formed by the hole transport host and the electron transport host in the emission layer EML. In this case, the triplet energy of the exiplex formed by the hole transport host and the electron transport host may correspond to T1, which is a gap between the LUMO energy level of the electron transport host and the HOMO energy level of the hole transport host.

In an embodiment, the triplet energy (T1) of the exiplex formed by the hole transport host and the electron transport host may be about 2.4 eV to about 3.0 eV. In some embodiments, the triplet energy of the exiplex may be a value smaller than the energy gap of each host material. Accordingly, the exiplex may have a triplet energy of about 3.0 eV or less, which is the energy gap between the hole transport host and the electron transport host.

In some embodiments, at least one emission layer EML may include a quantum dot material. The core of the quantum dot may be selected from a Group II-VI compound, a Group III-VI compound, a Group I-III-VI compound, a Group III-V compound, a Group III-II-V compound, a Group IV-VI compound, a Group IV compound, a Group IV compound, and combinations thereof.

The Group II-VI compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The Group III-V compound may include a binary compound such as In₂S₃, and/or In₂Se₃, a ternary compound such as InGaS₃, and/or InGaSe₃, or one or more optional combinations thereof.

The Group I-III-VI compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂, and CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In some embodiments, the Group III-V compound may further include a Group II metal. For example, InZnP, etc. may be selected as a Group III-II-V compound.

The Group IV-VI compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The Group IV element may be selected from the group consisting of Si, Ge, and a mixture thereof. The IV group compound may be a binary compound selected from the group consisting of SiC, SiGe, and a mixture thereof.

In this case, the binary compound, the ternary compound and/or the quaternary compound may be present at substantially uniform concentration in a particle or may be present at a partially different concentration distribution state in the same particle. In some embodiments, a core/shell structure in which one quantum dot is around (e.g., wraps) another quantum dot may be utilized. The interface of the core and the shell may have a concentration gradient in which the concentration of an element present in the shell is decreased toward the center of the core.

In some embodiments, the quantum dot may have the above-described core-shell structure including a core including a nanocrystal and a shell around (e.g., wrapping) the core. The shell of the quantum dot may play (e.g., serve) the role of a protection layer for preventing or reducing the chemical deformation of the core to maintain semiconductor properties and/or the role of a charging layer for imparting the quantum dot with electrophoretic properties. The shell may have a single layer or a multilayer structure. Examples of the shell of the quantum dot may include a metal or non-metal oxide, a semiconductor compound, and/or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, CO₃O₄ and/or NiO, or a ternary compound such as MgAl₂O₄, CoFe₂O₄, NiFe₂O₄ and/or CoMn₂O₄, but the present disclosure is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the present disclosure is not limited thereto.

The quantum dot may have a full width of half maximum (FWHM) of emission wavelength spectrum of about 45 nm or less, about 40 nm or less, or, about 30 nm or less. Within these ranges, color purity and/or color reproducibility may be improved. In some embodiments, light emitted via such quantum dot is emitted in all directions, and light view angle properties may be improved.

In some embodiments, the shape of the quantum dot may be generally utilized shapes in the art, without specific limitation. For example, the shape of spherical, pyramidal, multi-arm, or cubic nanoparticle, nanotube, nanowire, nanofiber, nanoplate particle, etc. may be utilized.

The quantum dot may control the color of light emitted according to the particle size, and accordingly, the quantum dot may have one or more suitable emission colors such as blue, red and green.

In the light emitting elements ED of embodiments, as shown in FIG. 3 to FIG. 6, the electron transport region ETR is provided on the emission layer EML. The electron transport region ETR may include at least one of a hole blocking layer HBL, an electron transport layer ETL or an electron injection layer EIL. However, the present disclosure is not limited thereto.

The electron transport region ETR may have a single layer formed utilizing a single material, a single layer formed utilizing multiple different materials, or a multilayer structure having multiple layers formed utilizing multiple different materials.

For example, the electron transport region ETR may have a single layer structure of an electron injection layer EIL or an electron transport layer ETL, or a single layer structure formed utilizing an electron injection material and an electron transport material. Further, the electron transport region ETR may have a single layer structure formed utilizing multiple different materials, or a structure stacked from the emission layer EML of electron transport layer ETL/electron injection layer EIL, hole blocking layer HBL/electron transport layer ETL/electron injection layer EIL, or electron transport layer ETL/buffer layer/electron injection layer EIL, but the present disclosure is not limited thereto. The thickness of the electron transport region ETR may be, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1.

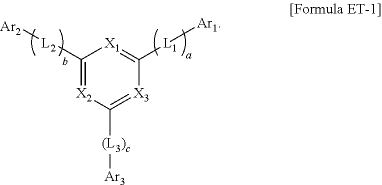

In Formula ET-1, at least one selected from among X₁ to X₃ may be N, and the remainder may be CRₐ. Rₐ may be a hydrogen atom, a deuterium atom, a substituted or unsubstituted alkyl of 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-1, “a” to “c” may each independently be an integer of 0 to 10. In Formula ET-1, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when “a” to “c” are integers of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), and mixtures thereof, but the present disclosure is not limited thereto.

The electron transport region ETR may include at least one selected from among Compounds ET1 to ET36.

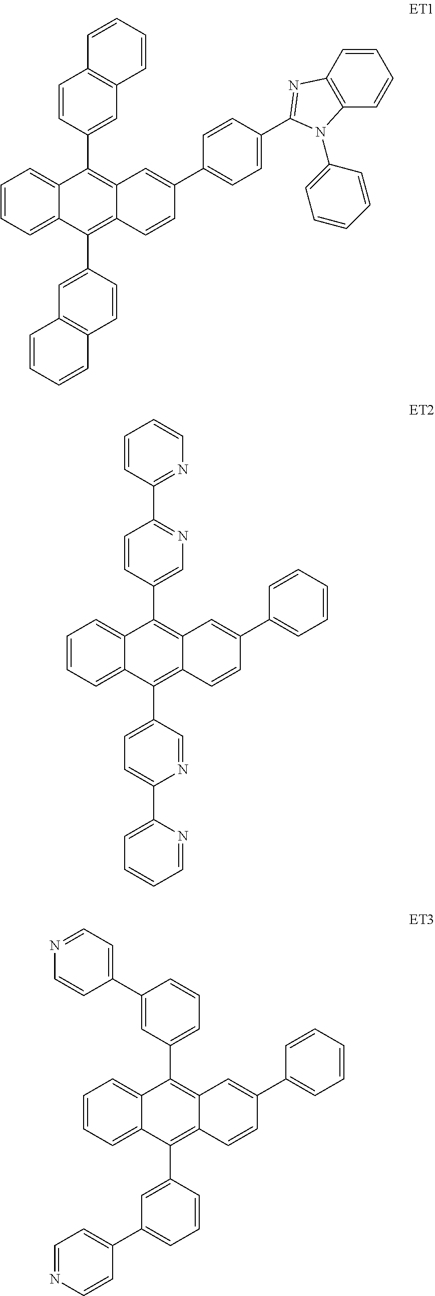 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

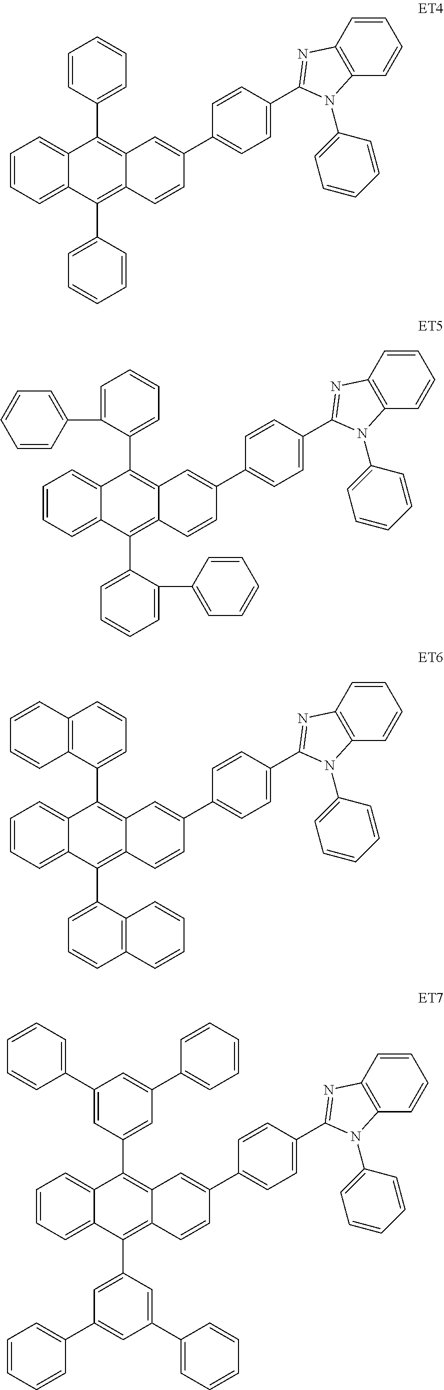 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

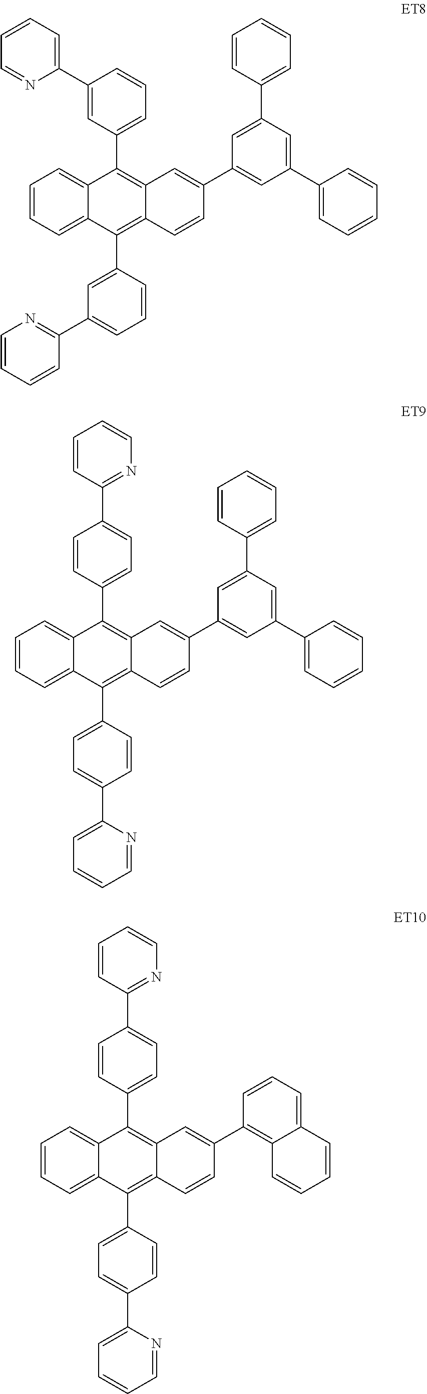 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

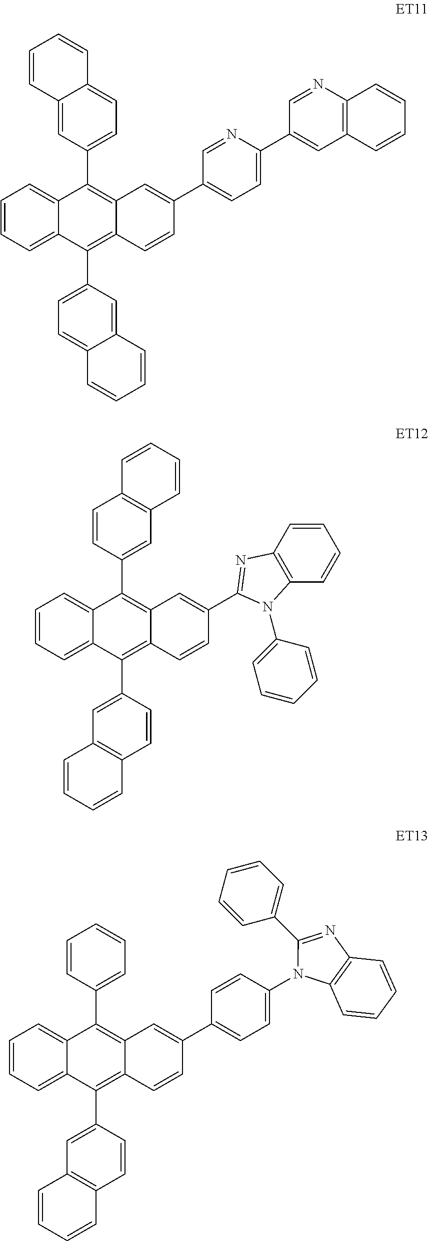 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

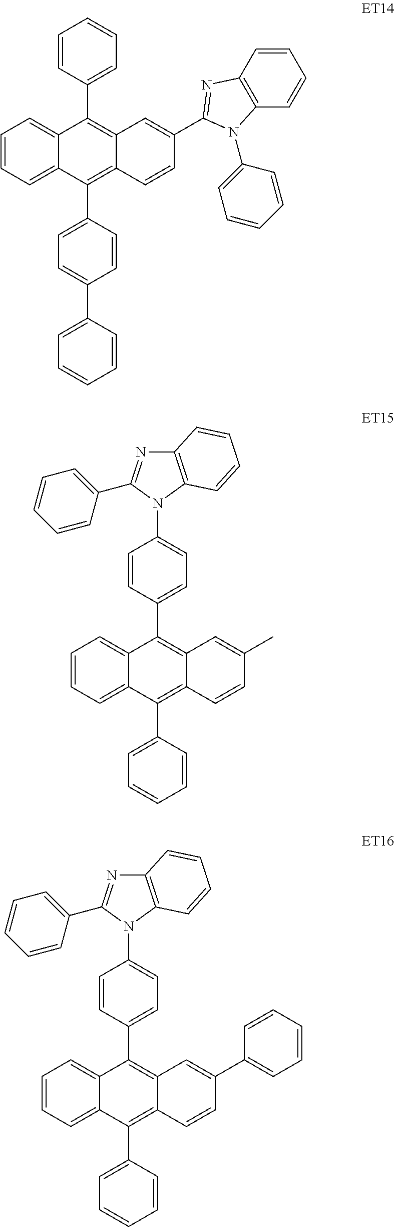 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

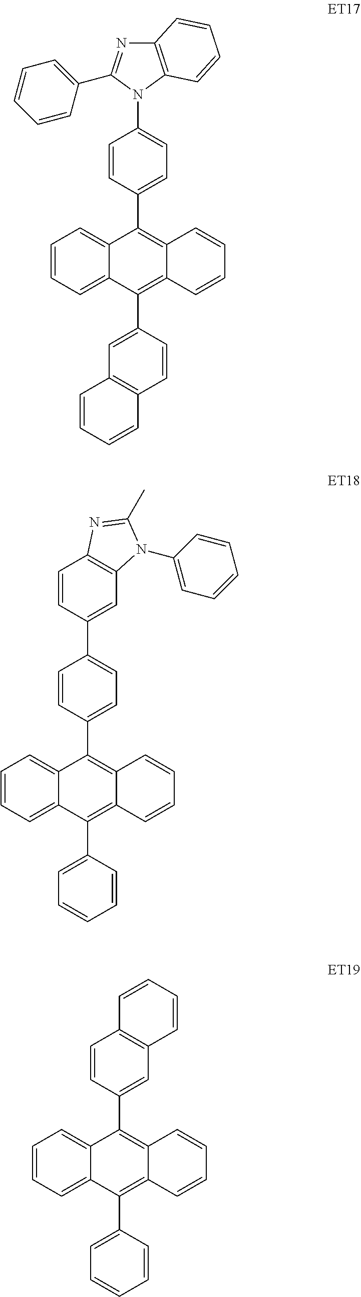

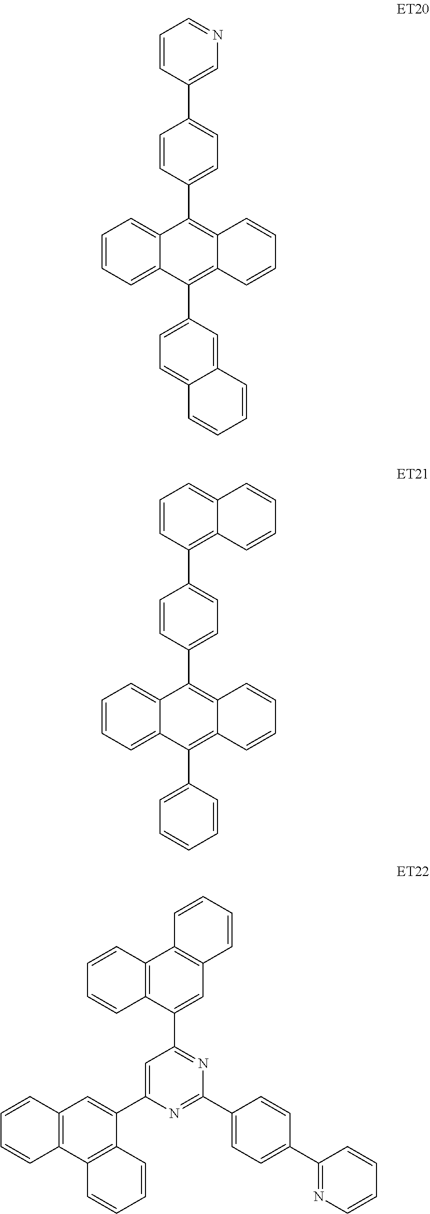

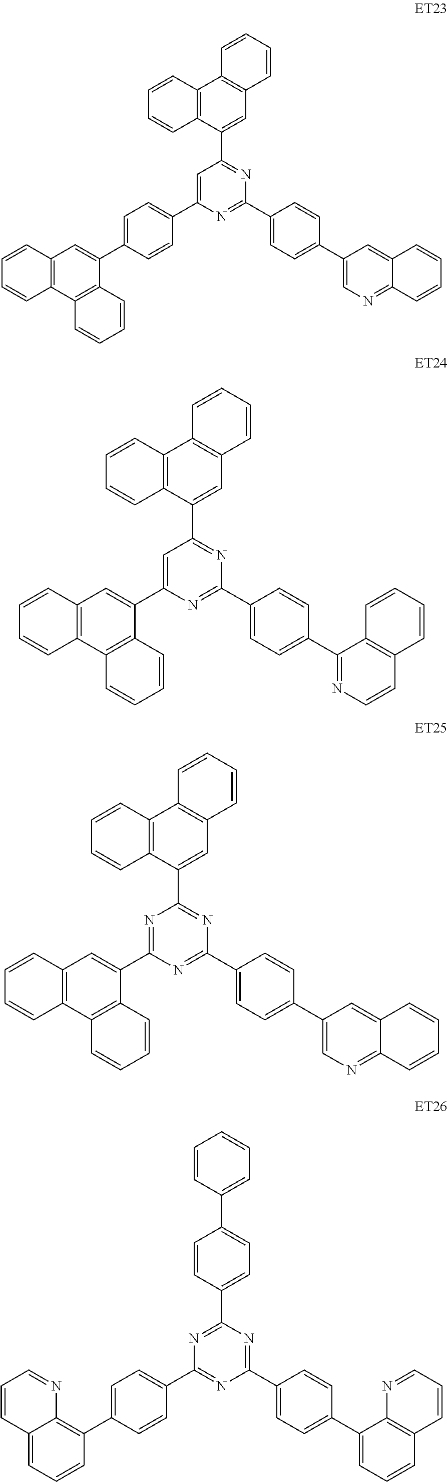 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

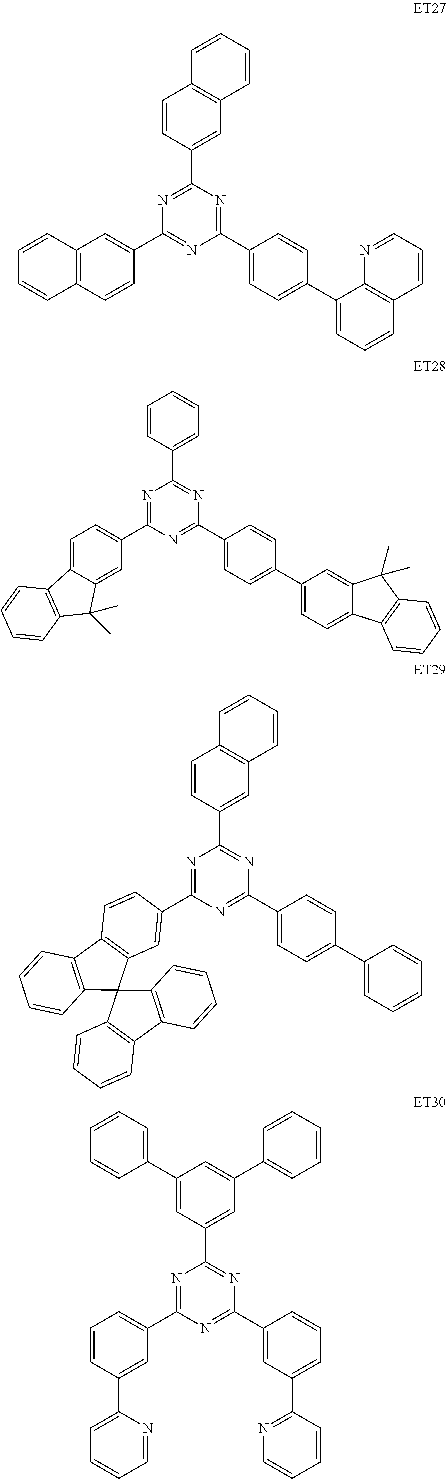

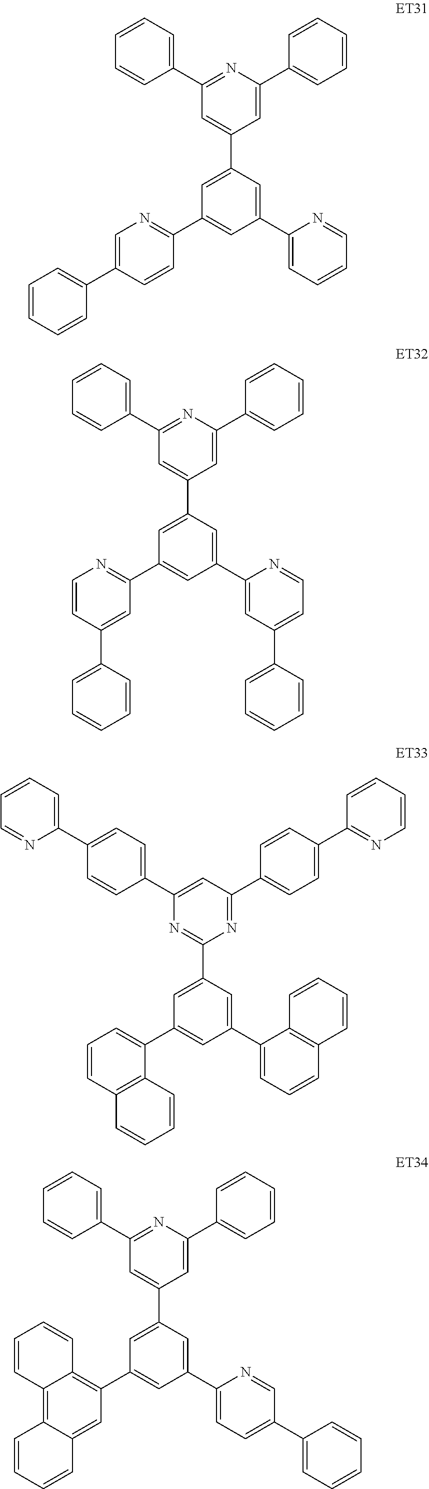 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

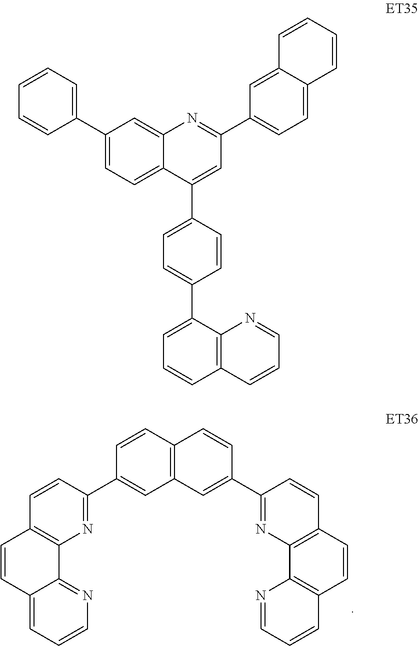

In some embodiments, the electron transport region ETR may include a metal halide such as LiF, NaCl, CsF, RbCl, RbI, CuI and/or KI, a lanthanide metal such as Yb, or a co-depositing material of the metal halide and the lanthanide metal. For example, the electron transport region ETR may include KI:Yb, RbI:Yb, LiF:Yb, etc., as the co-depositing material. In some embodiments, the electron transport region ETR may utilize a metal oxide such as Li₂O and/or BaO, or 8-hydroxy-lithium quinolate (Liq). However, the present disclosure is not limited thereto. The electron transport region ETR also may be formed utilizing a mixture material of an electron transport material and an insulating organo metal salt. The organo metal salt may be a material having an energy band gap of about 4 eV or more. For example, the organo metal salt may include, for example, one or more metal acetates, metal benzoates, metal acetoacetates, metal acetylacetonates, and/or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, the present disclosure is not limited thereto.

The electron transport region ETR may include the compounds of the electron transport region in at least one selected from among an electron injection layer EIL, an electron transport layer ETL, and a hole blocking layer HBL.

When the electron transport region ETR includes the electron transport layer ETL, the thickness of the electron transport layer ETL may be from about 100 Å to about 1,000 Å, for example, from about 150 Å to about 500 Å. When the thickness of the electron transport layer ETL satisfies the above-described ranges, satisfactory electron transport properties may be obtained without substantial increase of a driving voltage. When the electron transport region ETR includes the electron injection layer EIL, the thickness of the electron injection layer EIL may be from about 1 Å to about 100 Å, or from about 3 Å to about 90 Å. When the thickness of the electron injection layer EIL satisfies the above described ranges, satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but the present disclosure is not limited thereto. For example, when the first electrode EL1 is an anode, the second cathode EL2 may be a cathode, and when the first electrode EL1 is a cathode, the second electrode EL2 may be an anode. The second electrode EL2 may include at least one selected from among Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, and/or oxides thereof.

The second electrode EL2 may be a transmissive electrode, a transflective electrode or a reflective electrode. When the second electrode EL2 is the transmissive electrode, the second electrode EL2 may include a transparent metal oxide, for example, ITO, IZO, ZnO, ITZO, etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, W, one or more compounds thereof, or one or more mixtures thereof (for example, AgMg, AgYb, or MgYb). In some embodiments, the second electrode EL2 may have a multilayered structure including a reflective layer or a transflective layer formed utilizing the above-described materials and a transparent conductive layer formed utilizing ITO, IZO, ZnO, ITZO, etc. For example, the second electrode EL2 may include one or more of the aforementioned metal materials, combinations of two or more metal materials selected from the aforementioned metal materials, and/or oxides of the aforementioned metal materials.

In some embodiments, the second electrode EL2 may be connected with an auxiliary electrode. When the second electrode EL2 is connected with the auxiliary electrode, the resistance of the second electrode EL2 may decrease.

In some embodiments, on the second electrode EL2 in the light emitting element ED of an embodiment, a capping layer CPL may be further disposed. The capping layer CPL may include a multilayer or a single layer.

In an embodiment, the capping layer CPL may be an organic layer or an inorganic layer. For example, when the capping layer CPL includes an inorganic material, the inorganic material may include an alkali metal compound such as LiF, an alkaline earth metal compound such as MgF₂, SiON, SiNₓ, SiO_{y}, etc.

For example, when the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc., or may include an epoxy resin, and/or acrylate such as methacrylate. In some embodiments, a capping layer CPL may include at least one selected from among Compounds P1 to P5, but the present disclosure is not limited thereto.

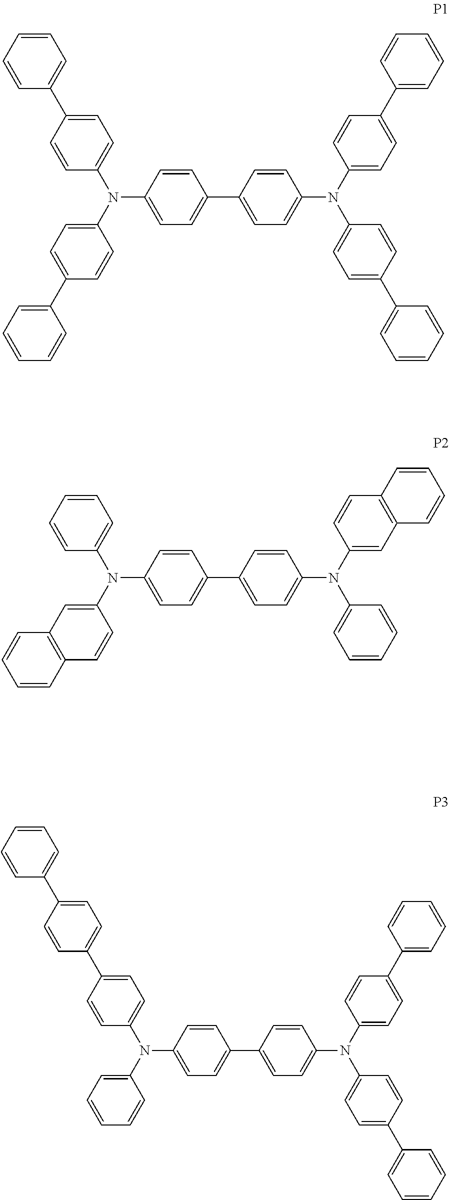 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

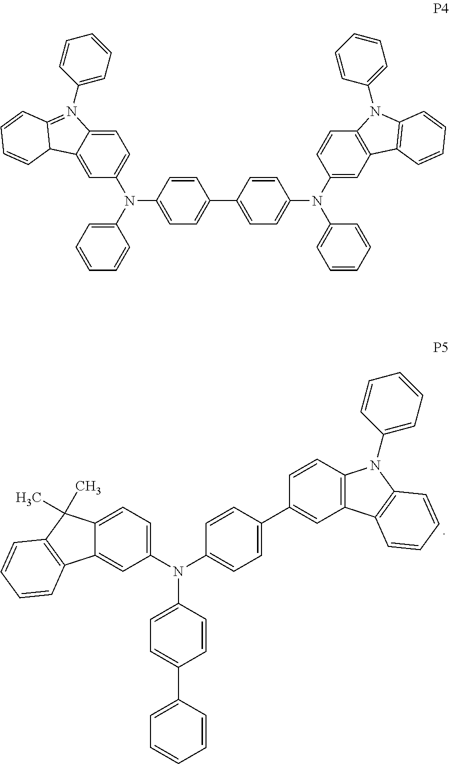

In some embodiments, the refractive index of the capping layer CPL may be about 1.6 or more. For example, the refractive index of the capping layer CPL with respect to light in a wavelength range of about 550 nm to about 660 nm may be about 1.6 or more.

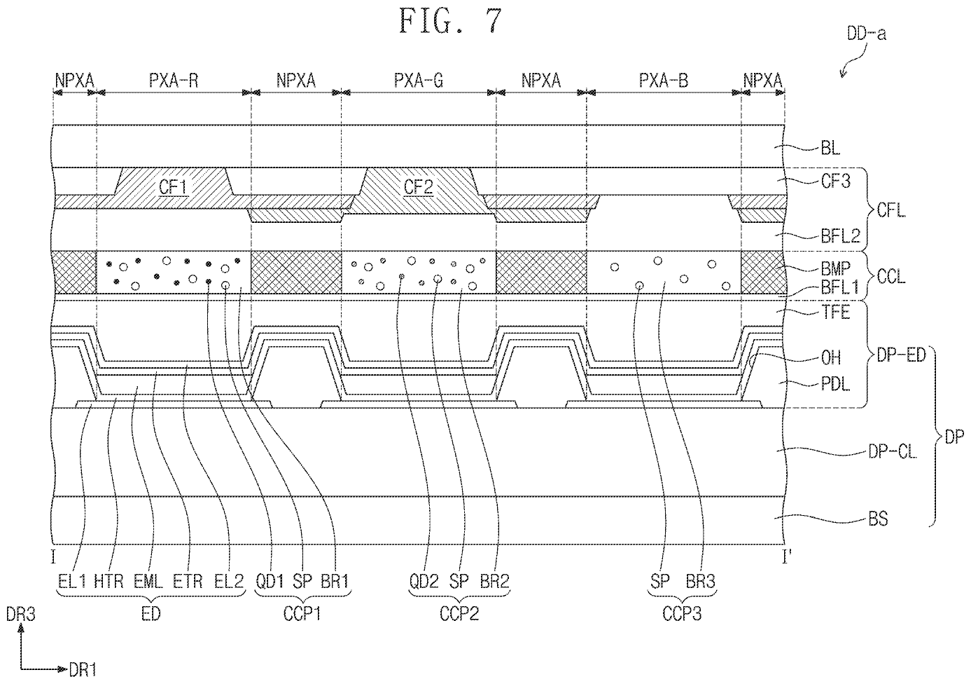

FIG. 7-FIG. 10 are cross-sectional views on display apparatuses according to embodiments, respectively. In the explanation on the display apparatuses of embodiments, referring to FIG. 7-FIG. 10, the overlapping parts with the explanation on FIG. 1-FIG. 6 will not be explained again, and the different features will be explained chiefly.

Referring to FIG. 7, a display apparatus DD-a according to an embodiment may include a display panel DP including a display element layer DP-ED, a light controlling layer CCL disposed on the display panel DP, and a color filter layer CFL.

In an embodiment shown in FIG. 7, the display panel DP includes a base layer BS, a circuit layer DP-CL provided on the base layer BS and a display element layer DP-ED, and the display element layer DP-ED may include a light emitting element ED.

The light emitting element ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In some embodiments, the same structures of the light emitting elements of FIG. 3-FIG. 6 may be applied to the structure of the light emitting element ED shown in FIG. 7.

The hole transport region HTR of the light emitting element ED included in the display apparatus DD-a according to an embodiment may include the amine compound of an embodiment, described above.

Referring to FIG. 7, the emission layer EML may be disposed in an opening portion OH defined in a pixel definition layer PDL. For example, the emission layer EML divided by the pixel definition layer PDL and correspondingly provided to each of luminous areas PXA-R, PXA-G and PXA-B may be to emit light in substantially the same wavelength region. In the display apparatus DD of an embodiment, the emission layer EML may be to emit blue light. In some embodiments, different from the drawings, in an embodiment, the emission layer EML may be provided as a common layer for all luminous areas PXA-R, PXA-G and PXA-B.

The light controlling layer CCL may be disposed on the display panel DP. The light controlling layer CCL may include a light converter. The light converter may be a quantum dot or a phosphor. The light converter may transform the wavelength of light provided and then emit (e.g., emit a different color light). For example, the light controlling layer CCL may be a layer including a quantum dot or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 7, a partition pattern BMP may be disposed between the separated light controlling parts CCP1, CCP2 and CCP3, but the present disclosure is not limited thereto. In FIG. 7, the partition pattern BMP is shown not to be overlapped with the light controlling parts CCP1, CCP2 and CCP3, but in some embodiments, at least a portion of the edge of the light controlling parts CCP1, CCP2 and CCP3 may be overlapped with the partition pattern BMP.

The light controlling layer CCL may include a first light controlling part CCP1 including a first quantum dot QD1 converting a first color light provided from the light emitting element ED into a second color light, a second light controlling part CCP2 including a second quantum dot QD2 converting the first color light into a third color light, and a third light controlling part CCP3 transmitting the first color light.

In an embodiment, the first light controlling part CCP1 may provide red light which is the second color light, and the second light controlling part CCP2 may provide green light which is the third color light. The third color controlling part CCP3 may be to transmit and provide blue light which is the first color light provided from the light emitting element ED. For example, the first quantum dot QD1 may be a red quantum dot, and the second quantum dot QD2 may be a green quantum dot. For the quantum dots QD1 and QD2, the same contents as those described above may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a quantum dot (e.g., not include any quantum dot) but may include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected from among TiO₂, ZnO, Al₂O₃, SiO₂, and hollow silica.

Each of the first light controlling part CCP1, the second light controlling part CCP2, and the third light controlling part CCP3 may include a corresponding one of the base resins BR1, BR2 and BR3, dispersing the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2 and BR3 may be one or more acrylic resins, urethane-based resins, silicone-based resins, epoxy-based resins, etc. The base resins BR1, BR2 and BR3 may be transparent resins. In an embodiment, the first base resin BR1, the second base resin BR2 and the third base resin BR3 may be the same or different from each other.

The light controlling layer CCL may include a barrier layer BFL1. The barrier layer BFL1 may play the role of blocking or reducing the penetration of moisture and/or oxygen (hereinafter, will be referred to as “humidity/oxygen”). The barrier layer BFL1 may be disposed on the light controlling parts CCP1, CCP2 and CCP3 to block or reduce the exposure of the light controlling parts CCP1, CCP2 and CCP3 to humidity/oxygen. In some embodiments, the barrier layer BFL1 may cover the light controlling parts CCP1, CCP2 and CCP3. In some embodiments, the barrier layer BFL2 may be provided between a color filter layer CFL and the light controlling parts CCP1, CCP2 and CCP3.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, and/or a metal thin film for securing light transmittance. In some embodiments, the barrier layers BFL1 and BFL2 may further include an organic layer. The barrier layers BFL1 and BFL2 may be composed of a single layer of multiple layers.

In the display apparatus DD-a of an embodiment, the color filter layer CFL may be disposed on the light controlling layer CCL. For example, the color filter layer CFL may be disposed directly on the light controlling layer CCL. In this case, the barrier layer BFL2 may not be provided.

The color filter layer CFL may include filters CF1, CF2 and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment and/or dye. The first filter CF1 may include a red pigment and/or dye, the second filter CF2 may include a green pigment and/or dye, and the third filter CF3 may include a blue pigment and/or dye. In some embodiments, the present disclosure is not limited thereto, and the third filter CF3 may not include (e.g., may exclude) the pigment or dye. The third filter CF3 may include a polymer photosensitive resin and not include a pigment or dye (e.g., any pigment or dye). The third filter CF3 may be transparent. The third filter CF3 may be formed utilizing a transparent photosensitive resin.

In some embodiments, in an embodiment, the first filter CF1 and the second filter CF2 may be yellow filters. The first filter CF1 and the second filter CF2 may be provided in one body without distinction. Each of the first to third filters CF1, CF2 and CF3 may be disposed corresponding to a corresponding one of a red luminous area PXA-R, green luminous area PXA-G, and blue luminous area PXA-B.

In some embodiments, the color filter layer CFL may include a light blocking part. The color filter layer CFL may include the light blocking part disposed to overlap with the boundaries of the neighboring filters CF1, CF2 and CF3. The light blocking part may be a black matrix. The light blocking part may be formed by including an organic light blocking material and/or an inorganic light blocking material, including a black pigment and/or black dye. The light blocking part may divide the boundaries selected from among adjacent filters CF1, CF2 and CF3. In some embodiments, the light blocking part may be formed as a blue filter.

On the color filter layer CFL, a base substrate BL may be disposed. The base substrate BL may be a member providing a base surface on which the color filter layer CFL, the light controlling layer CCL, etc. are disposed. The base substrate BL may be a glass substrate, a metal substrate, a plastic substrate, etc. However, the present disclosure is not limited thereto, and the base substrate BL may be an inorganic layer, an organic layer or a composite material layer. In some embodiments, different from the drawing, the base substrate BL may not be provided.

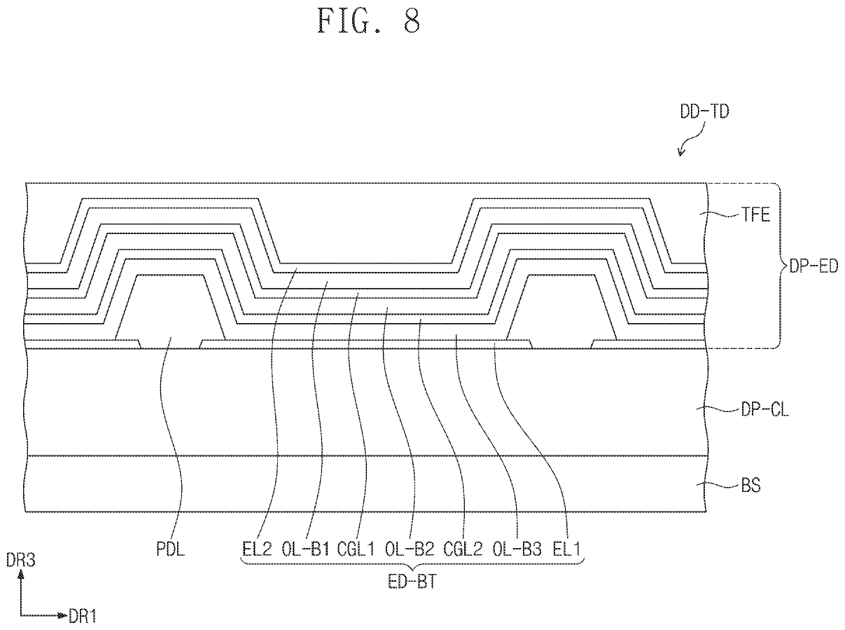

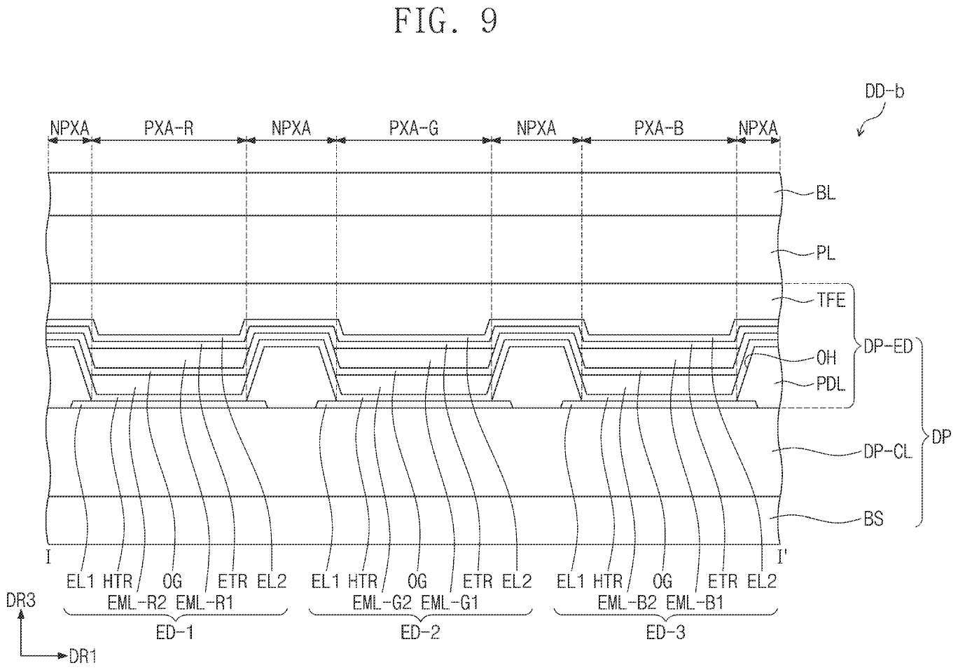

FIG. 8 is a cross-sectional view showing a portion of the display apparatus according to an embodiment. In FIG. 8, the cross-sectional view of a portion corresponding to the display panel DP in FIG. 7 is shown. In a display apparatus DD-TD of an embodiment, the light emitting element ED-BT may include multiple light emitting structures OL-B1, OL-B2 and OL-B3. The light emitting element ED-BT may include oppositely disposed first electrode EL1 and second electrode EL2, and the multiple light emitting structures OL-B1, OL-B2 and OL-B3 stacked in the stated order in a thickness direction and provided between the first electrode EL1 and the second electrode EL2. Each of the light emitting structures OL-B1, OL-B2 and OL-B3 may include an emission layer EML (FIG. 7), a hole transport region HTR and an electron transport region ETR disposed with the emission layer EML (FIG. 7) therebetween.

For example, the light emitting element ED-BT included in the display apparatus DD-TD of an embodiment may be a light emitting element of a tandem structure including multiple emission layers.

In an embodiment shown in FIG. 8, light emitted from the light emitting structures OL-B1, OL-B2 and OL-B3 may be all blue light. However, the present disclosure is not limited thereto, and the wavelength regions of light emitted from the light emitting structures OL-B1, OL-B2 and OL-B3 may be different from each other. For example, the light emitting element ED-BT including the multiple light emitting structures OL-B1, OL-B2 and OL-B3 emitting light in different wavelength regions may be to emit white light.

Between neighboring light emitting structures OL-B1, OL-B2 and OL-B3, charge generating layers CGL1 and CGL2 may be respectively disposed. The charge generating layers CGL1 and CGL2 may include a p-type or kind charge generating layer and/or an n-type or kind charge generating layer.

In at least one selected from among the light emitting structures OL-B1, OL-B2 and OL-B3, included in the display apparatus DD-TD of an embodiment, the amine compound of an embodiment may be included.

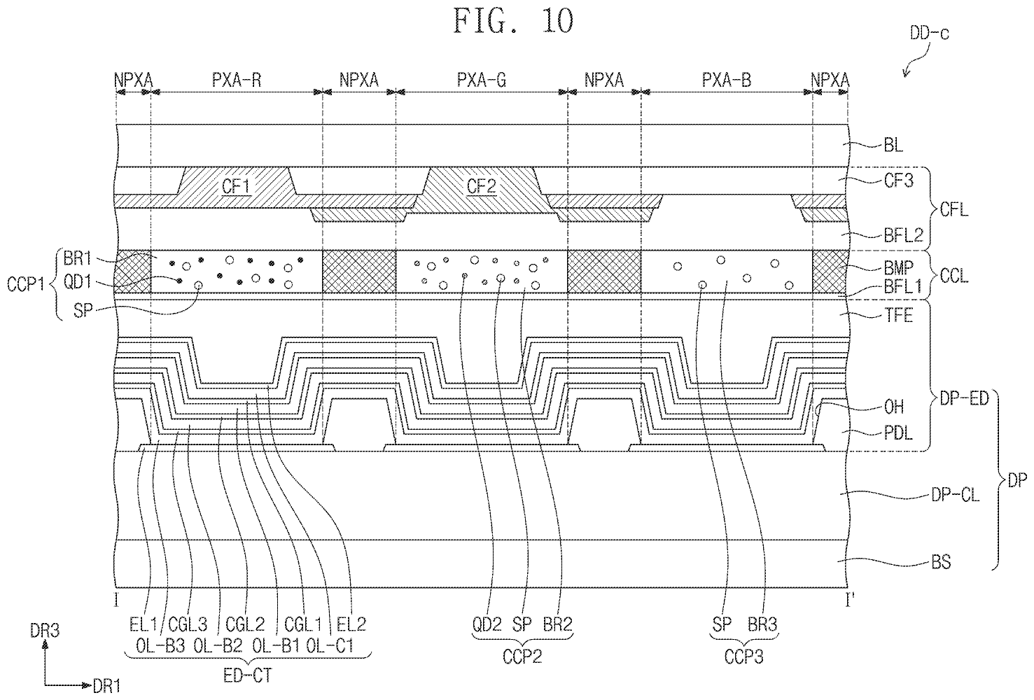

Referring to FIG. 9, a display apparatus DD-b according to an embodiment may include light emitting elements ED-1, ED-2 and ED-3, each formed by stacking two emission layers. Compared to the display apparatus DD of an embodiment, shown in FIG. 2, an embodiment shown in FIG. 10 is different in that the first to third light emitting elements ED-1, ED-2 and ED-3 each include two emission layers stacked in a thickness direction. In the first to third light emitting elements ED-1, ED-2 and ED-3, the two emission layers may be to emit light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and between the first blue emission layer EML-B1 and the second blue emission layer EML-B2, an emission auxiliary part OG may be disposed.

The emission auxiliary part OG may include a single layer or a multilayer. The emission auxiliary part OG may include a charge generating layer. For example, the emission auxiliary part OG may include an electron transport region, a charge generating layer, and a hole transport region stacked in the stated order. The emission auxiliary part OG may be provided as a common layer in all of the first to third light emitting elements ED-1, ED-2 and ED-3. However, the present disclosure is not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be disposed between the electron transport region ETR and the emission auxiliary part OG. The second red emission layer EML-R2, the second green emission layer EML-G2 and the second blue emission layer EML-B2 may be disposed between the emission auxiliary part OG and the hole transport region HTR.

For example, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in the stated order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in the stated order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in the stated order.

In some embodiments, an optical auxiliary layer PL may be disposed on a display element layer DP-ED. The optical auxiliary layer PL may include a polarization layer. The optical auxiliary layer PL may be disposed on a display panel DP and may control reflected light at the display panel DP by external light. Different from the drawings, the optical auxiliary layer PL may not be provided from the display apparatus according to an embodiment.

Different from FIG. 8 and FIG. 9, a display apparatus DD-c in FIG. 10 is shown to include four light emitting structures OL-B1, OL-B2, OL-B3 and OL-C1. A light emitting element ED-CT may include oppositely disposed first electrode EL1 and second electrode EL2, and first to fourth light emitting structures OL-B1, OL-B2, OL-B3 and OL-C1 stacked in the stated order in a thickness direction between the first electrode EL1 and the second electrode EL2. Between adjacent first to fourth light emitting structures OL-B1, OL-B2, OL-B3 and OL-C1, charge generating layers CGL1, CGL2 and CGL3 may be respectively disposed. Selected from among the four light emitting structures, the first to third light emitting structures OL-B1, OL-B2 and OL-B3 may be to emit blue light, and the fourth light emitting structure OL-C1 may be to emit green light. However, the present disclosure is not limited thereto, and the first to fourth light emitting structures OL-B1, OL-B2, OL-B3 and OL-C1 may be to emit light of different wavelengths.

Charge generating layers CGL1, CGL2 and CGL3 disposed among neighboring light emitting structures OL-B1, OL-B2, OL-B3 and OL-C1 may each include a p-type or kind charge generating layer and/or an n-type or kind charge generating layer.

In at least one selected from among the light emitting structures OL-B1, OL-B2, OL-B3 and OL-C1, included in the display apparatus DD-c of an embodiment, the amine compound of an embodiment may be included.

The light emitting element ED according to an embodiment of the present disclosure may include the amine compound of an embodiment in at least one functional layer disposed between a first electrode EL1 and a second electrode EL2 to provide improved emission efficiency and improved life characteristics. The light emitting element ED according to an embodiment may include the amine compound of an embodiment in at least one selected from among a hole transport region HTR, an emission layer EML, and an electron transport region ETR, disposed between a first electrode EL1 and a second electrode EL2, and/or in a capping layer CPL.

For example, the amine compound according to an embodiment may be included in the hole transport region HTR of the light emitting element ED of an embodiment, and the light emitting element of an embodiment may show long-life characteristics.

The amine compound of an embodiment includes a benzocarbazole moiety for improving the stability of a radical or radical cation state, increasing intermolecular π-π interaction and improving hole transport capacity, and may contribute to the decrease of a driving voltage and increase of efficiency of a light emitting element. In some embodiments, the amine compound of an embodiment introduces (e.g., includes) at least one substituent selected from among a naphthyl group, a phenanthryl group, a benzoheterole group and a fluorenyl group, and may improve electron tolerance and exciton tolerance. Accordingly, the efficiency and life of a light emitting element including the amine compound of an embodiment may be improved.

Hereinafter, referring to examples and comparative examples, the amine compound according to an embodiment and the light emitting element according to an embodiment of the present disclosure will be explained in more details. In addition, the examples are described to assist the understanding of the present disclosure, but the scope of the present disclosure is not limited thereto.

### EXAMPLES

### 1. Synthesis of Amine Compounds

First, the synthetic methods of the amine compounds according to embodiments will be explained in more detail illustrating the synthetic methods of Compound A5, Compound A6, Compound B19, Compound B24, Compound C22, Compound C33, Compound D4, Compound D8, Compound D18, and Compound D67. In addition, the synthetic methods of the amine compounds explained hereinafter are embodiments, and the synthetic method of the amine compound according to an embodiment of the present disclosure is not limited to the embodiments below.

### (1) Synthesis of Compound A5

Amine Compound A5 according to an embodiment may be synthesized, for example, by the steps of Reaction 1.

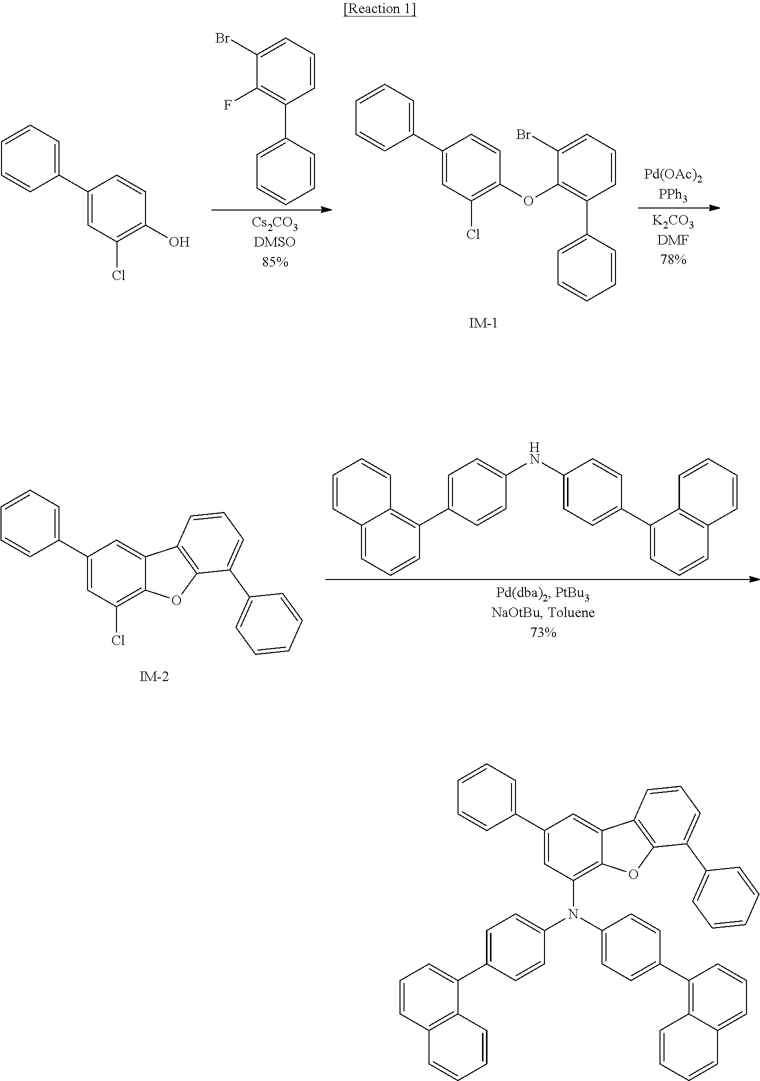

### 1) Synthesis of Intermediate Compound IM-1

To a 500 mL, three-neck flask, 3-chloro-[1,1′-biphenyl]-4-ol (15.00 g, 73.3 mmol), 3-bromo-2-fluoro-1,1′-biphenyl (22.09 g, 1.5 equiv, 88.0 mmol), Cs₂CO₃ (47.76 g, 2.0 equiv, 146.6 mmol) and 147 mL of DMSO were added in the stated order, followed by heating to about 100° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-1 (27.15 g, yield 85%).

By measuring FAB-MS, mass number of m/z=435 was observed as a molecular ion peak, and Intermediate Compound IM-1 was confirmed.

### 2) Synthesis of Intermediate Compound IM-2

Under an argon (Ar) atmosphere, to a 500 mL, three-neck flask, Intermediate Compound IM-1 (25.00 g, 57.4 mmol), Pd(OAc)₂ (0.64 g, 0.05 equiv, 2.9 mmol), K₂CO₃ (11.89 g, 1.5 equiv, 86.1 mmol), PPh₃ (1.50 g, 0.10 equiv, 5.7 mmol) and 286 mL of DMF were added in the stated order, followed by heating to about 110° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-2 (15.88 g, yield 78%).

By measuring FAB-MS, mass number of m/z=354 was observed as a molecular ion peak, and Intermediate Compound IM-2 was confirmed.

### 3) Synthesis of Compound A5

Under an argon (Ar) atmosphere, to a 300 mL, three-neck flask, bis[4-(naphthalen-1-yl)phenyl]amine (10.00 g, 23.7 mmol), Pd(dba)₂ (0.41 g, 0.03 equiv, 0.7 mmol), NaO^{t}Bu (4.56 g, 2.0 equiv, 47.4 mmol), 118 mL of toluene, Intermediate Compound IM-2 (9.26 g, 1.1 equiv, 26.1 mmol) and P^{t}Bu₃ (0.48 g, 0.1 equiv, 2.4 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound A5 (12.81 g, yield 73%) as a solid.

By measuring FAB-MS, mass number of m/z=739 was observed as a molecular ion peak, and Compound A5 was confirmed.

### (2) Synthesis of Compound A6

Amine Compound A6 according to an embodiment may be synthesized, for example, by the steps of Reaction 2.

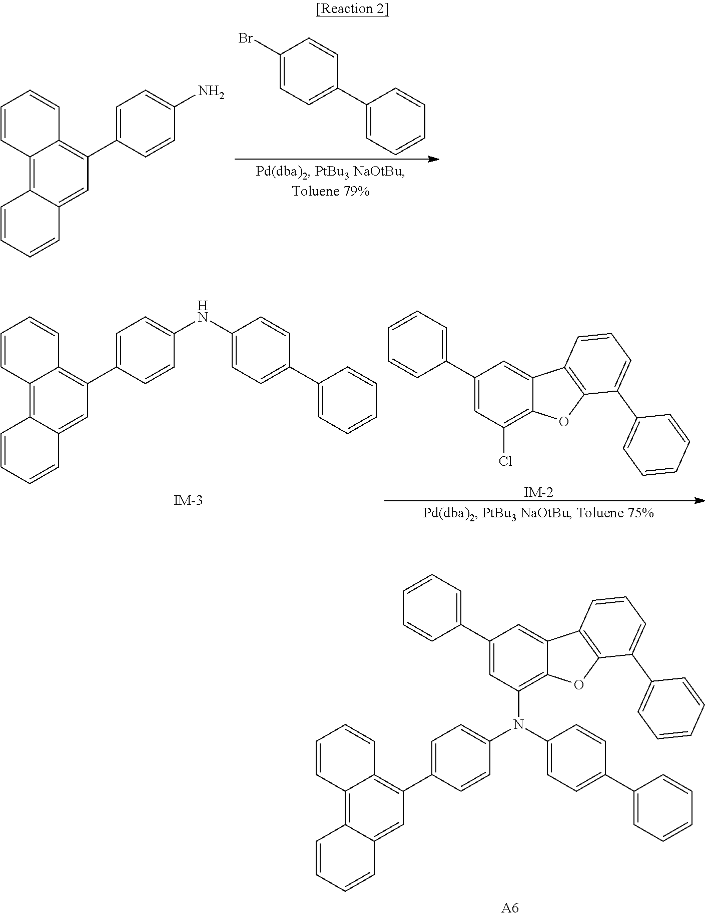

### 1) Synthesis of Intermediate Compound IM-3

Under an argon (Ar) atmosphere, to a 500 mL, three-neck flask, 4-(phenanthren-9-yl)aniline (15.00 g, 55.7 mmol), Pd(dba)₂ (0.96 g, 0.03 equiv, 1.7 mmol), NaO^{t}Bu (5.35 g, 1.0 equiv, 55.7 mmol), 278 mL of toluene, 4-bromobiphenyl (14.28 g, 1.1 equiv, 61.3 mmol) and P^{t}Bu₃ (1.13 g, 0.1 equiv, 5.6 mmol) were added in the stated order, followed by heating, refluxing and stirring. After cooling to room temperature, water was added to the reaction solution, and an organic layer was separately taken. Toluene was added to an aqueous solution, and an organic layer was further extracted. Organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-3 (18.55 g, yield 79%).

By measuring FAB-MS, mass number of m/z=421 was observed as a molecular ion peak, and Intermediate Compound IM-3 was confirmed.

### 2) Synthesis of Compound A6

Under an argon (Ar) atmosphere, to a 300 mL, three-neck flask, Intermediate Compound IM-3 (10.00 g, 23.7 mmol), Pd(dba)₂ (0.41 g, 0.03 equiv, 0.7 mmol), NaOtBu (4.56 g, 2.0 equiv, 47.4 mmol), 118 mL of toluene, IM-2 (11.37 g, 1.1 equiv, 26.1 mmol) and P^{t}Bu₃ (0.48 g, 0.1 equiv, 2.4 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound A6 (13.16 g, yield 75%) as a solid.

By measuring FAB-MS, mass number of m/z=739 was observed as a molecular ion peak, and Compound A6 was confirmed.

### (3) Synthesis of Compound B19

Amine Compound B19 according to an embodiment may be synthesized, for example, by the steps of Reaction 3.

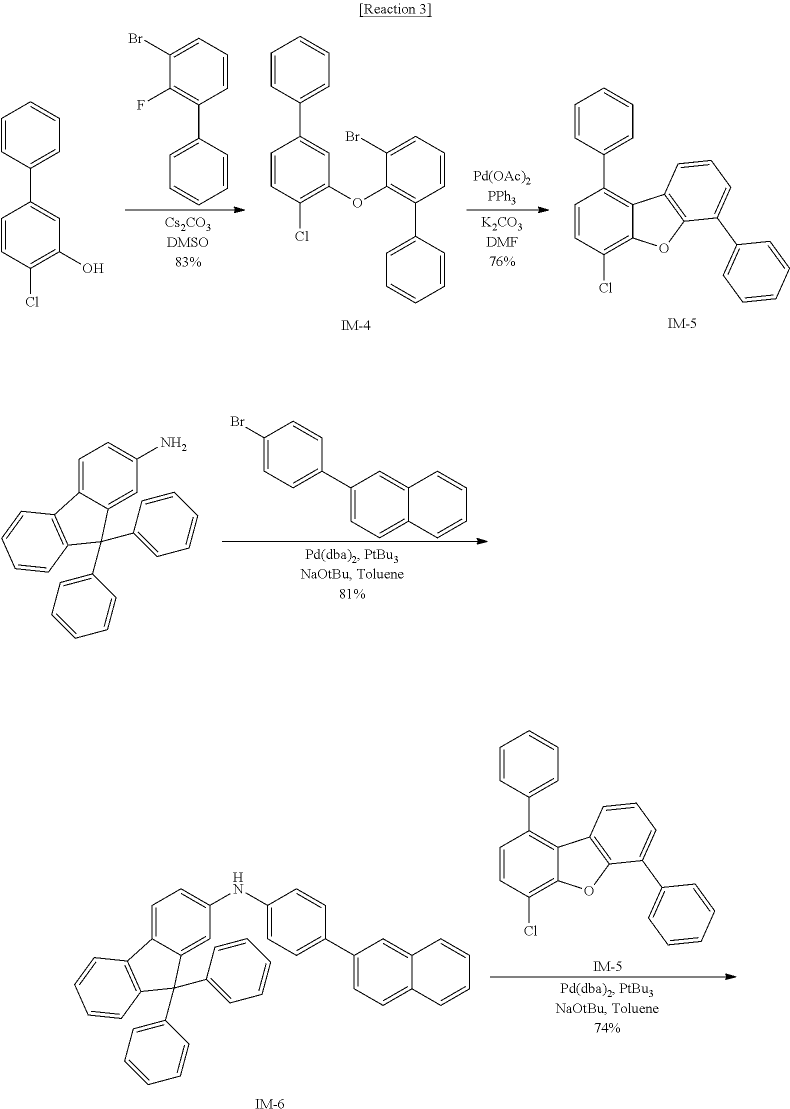

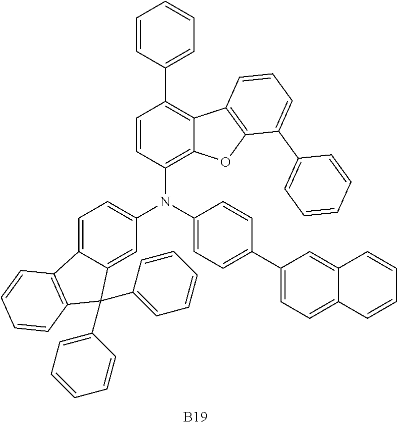

### 1) Synthesis of Intermediate Compound IM-4

Under an argon (Ar) atmosphere, to a 500 mL, three-neck flask, 4-chloro-[1,1′-biphenyl]-3-ol (15.00 g, 73.3 mmol), 3-bromo-2-fluoro-1,1′-biphenyl (22.09 g, 1.5 equiv, 88.0 mmol), Cs₂CO₃ (47.76 g, 2.0 equiv, 146.6 mmol) and 147 mL of DMSO were added in the stated order, followed by heating to about 100° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-4 (26.51 g, yield 83%).

By measuring FAB-MS, mass number of m/z=435 was observed as a molecular ion peak, and Intermediate Compound IM-4 was confirmed.

### 2) Synthesis of Intermediate Compound IM-5

Under an argon (Ar) atmosphere, to a 500 mL, three-neck flask, Intermediate Compound IM-4 (25.00 g, 57.4 mmol), Pd(OAc)₂ (0.64 g, 0.05 equiv, 2.9 mmol), K₂CO₃ (11.89 g, 1.5 equiv, 86.1 mmol), PPh₃ (1.50 g, 0.10 equiv, 5.7 mmol) and 286 mL of DMF were added in the stated order, followed by heating to about 110° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-5 (15.47 g, yield 76%).

By measuring FAB-MS, mass number of m/z=354 was observed as a molecular ion peak, and Intermediate Compound IM-5 was confirmed.

### 3) Synthesis of Intermediate Compound IM-6

Under an argon (Ar) atmosphere, to a 500 mL, three-neck flask, 9,9-diphenyl-9H-fluoren-2-amine (15.00 g, 45.0 mmol), Pd(dba)₂ (0.78 g, 0.03 equiv, 1.3 mmol), NaO^{t}Bu (4.32 g, 1.0 equiv, 45.0 mmol), 225 mL of toluene, 2-(4-bromophenyl)naphthalene (14.01 g, 1.1 equiv, 49.5 mmol) and P^{t}Bu₃ (0.91 g, 0.1 equiv, 4.5 mmol) were added in the stated order, followed by heating, refluxing and stirring. After cooling to room temperature, water was added to the reaction solution, and an organic layer was separately taken. Toluene was added to an aqueous layer, an organic layer was further extracted, and organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-6 (19.52 g, yield 81%).

By measuring FAB-MS, mass number of m/z=535 was observed as a molecular ion peak, and Intermediate Compound IM-6 was confirmed.

### 4) Synthesis of Compound B19

Under an argon (Ar) atmosphere, to a 300 mL, three-neck flask, Intermediate Compound IM-6 (10.00 g, 18.7 mmol), Pd(dba)₂ (0.32 g, 0.03 equiv, 0.6 mmol), NaO^{t}Bu (3.59 g, 2.0 equiv, 37.3 mmol), 93 mL of toluene, Intermediate Compound IM-5 (7.29 g, 1.1 equiv, 20.5 mmol) and P^{t}Bu₃ (0.38 g, 0.1 equiv, 1.9 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound B19 (11.80 g, yield 74%) as a solid.

By measuring FAB-MS, mass number of m/z=854 was observed as a molecular ion peak, and Compound B19 was confirmed.

### (4) Synthesis of Compound B24

Amine Compound B24 according to an embodiment may be synthesized, for example, by the steps of Reaction 4.

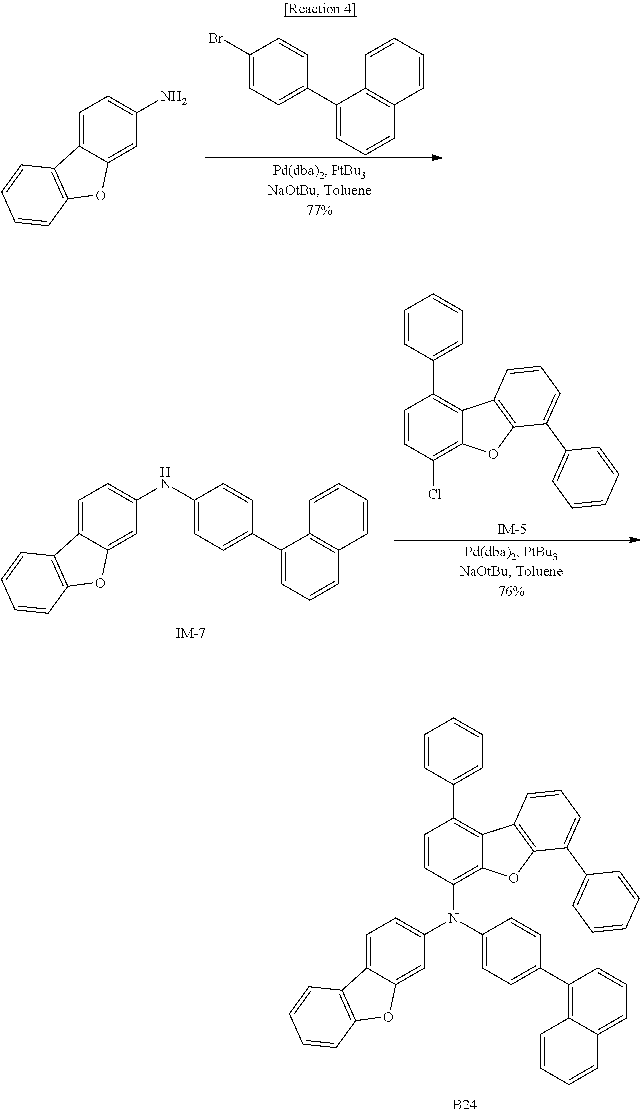

### 1) Synthesis of Intermediate Compound IM-7

Under an argon (Ar) atmosphere, to a 1000 mL, three-neck flask, dibenzofuran-3-amine (15.00 g, 81.9 mmol), Pd(dba)₂ (1.41 g, 0.03 equiv, 2.5 mmol), NaOtBu (7.87 g, 1.0 equiv, 81.9 mmol), 409 mL of toluene, 1-(4-bromophenyl)naphthalene (25.50 g, 1.1 equiv, 90.1 mmol) and P^{t}Bu₃ (1.66 g, 0.1 equiv, 8.2 mmol) were added in the stated order, followed by heating, refluxing and stirring. After cooling to room temperature, water was added to the reaction solution, and an organic layer was separately taken. Toluene was added to an aqueous solution, and an organic layer was further extracted. Organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-7 (24.30 g, yield 77%).

By measuring FAB-MS, mass number of m/z=385 was observed as a molecular ion peak, and Intermediate Compound IM-7 was confirmed.

### 2) Synthesis of Compound B24

Under an argon (Ar) atmosphere, to a 300 mL, three-neck flask, Intermediate Compound IM-7 (10.00 g, 25.9 mmol), Pd(dba)₂ (0.45 g, 0.03 equiv, 0.8 mmol), NaO^{t}Bu (4.99 g, 2.0 equiv, 51.9 mmol), 130 mL of toluene, Intermediate Compound IM-5 (10.13 g, 1.1 equiv, 28.5 mmol) and P^{t}Bu₃ (0.52 g, 0.1 equiv, 2.6 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound B24 (13.88 g, yield 76%) as a solid.

By measuring FAB-MS, mass number of m/z=703 was observed as a molecular ion peak, and Compound B24 was confirmed.

### (5) Synthesis of Compound C22

Amine Compound C22 according to an embodiment may be synthesized, for example, by the steps of Reaction 5.

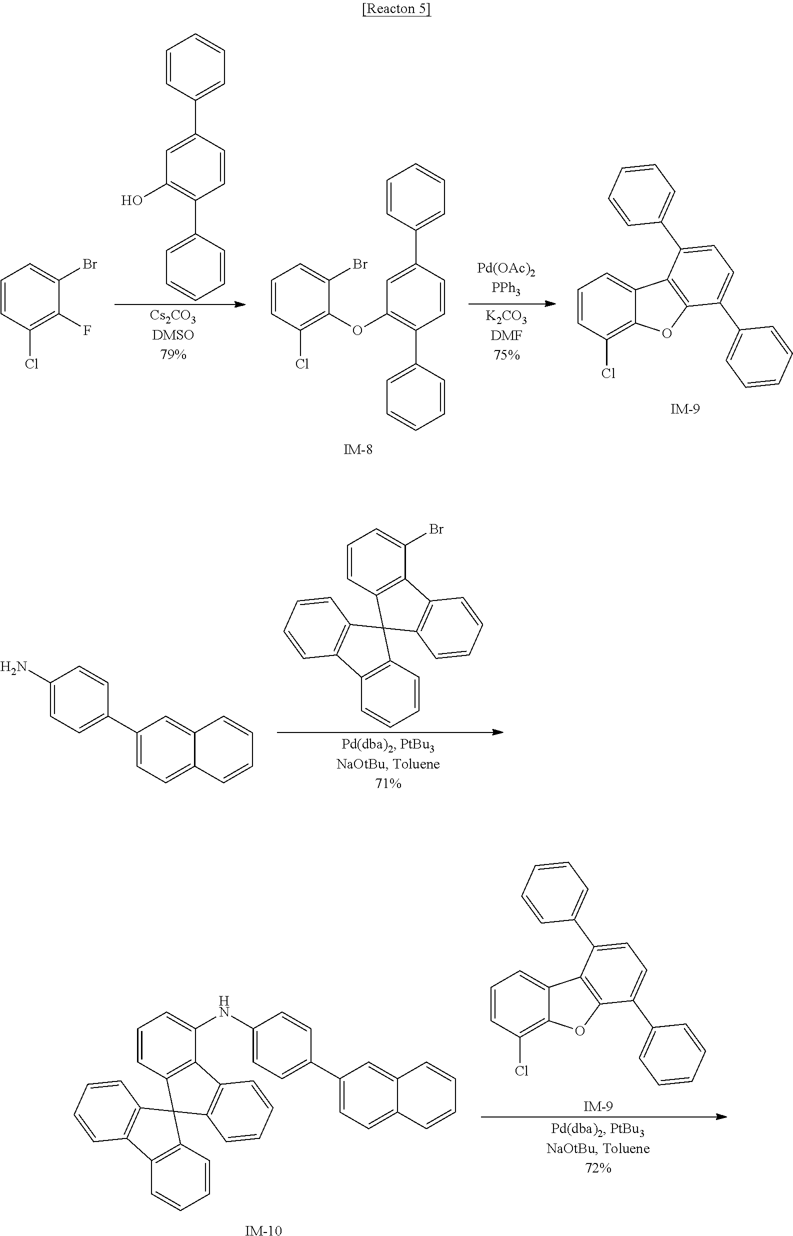

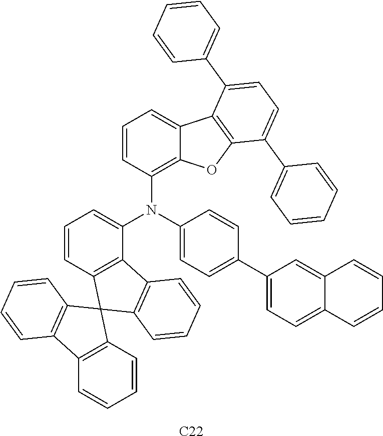 [SMILES: c1ccc(-c2ccc(-c3ccccc3)c3c2oc2c(N(c4ccc(-c5ccc6ccccc6c5)cc4)c4cccc5c4-c4ccccc4C54c5ccccc5-c5ccccc54)cccc23)cc1 ; "C22"]

### 1) Synthesis of Intermediate Compound IM-8

Under an argon (Ar) atmosphere, to a 500 mL, three-neck flask, [1,1′:4′,1″-terphenyl]-2′-ol (20.00 g, 81.2 mmol), 1-bromo-3-chloro-2-fluorobenzene (20.41 g, 1.5 equiv, 97.4 mmol), Cs₂CO₃ (52.91 g, 2.0 equiv, 162.4 mmol) and 162 mL of DMSO were added in the stated order, followed by heating to about 100° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-8 (27.95 g, yield 79%).

By measuring FAB-MS, mass number of m/z=435 was observed as a molecular ion peak, and Intermediate Compound IM-8 was confirmed.

### 2) Synthesis of Intermediate Compound IM-9

Under an argon (Ar) atmosphere, to a 500 mL, three-neck flask, Intermediate Compound IM-8 (25.00 g, 57.4 mmol), Pd(OAc)₂ (0.64 g, 0.05 equiv, 2.9 mmol), K₂CO₃ (11.89 g, 1.5 equiv, 86.1 mmol), PPh₃ (1.50 g, 0.10 equiv, 5.7 mmol) and 286 mL of DMF were added in the stated order, followed by heating to about 110° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-9 (15.27 g, yield 75%).

By measuring FAB-MS, mass number of m/z=354 was observed as a molecular ion peak, and Intermediate Compound IM-9 was confirmed.

### 3) Synthesis of Intermediate Compound IM-10

Under an argon (Ar) atmosphere, to a 1000 mL, three-neck flask, 4-(naphthalen-2-yl)aniline (15.00 g, 68.4 mmol), Pd(dba)₂ (1.18 g, 0.03 equiv, 2.1 mmol), NaO^{t}Bu (6.57 g, 1.0 equiv, 68.4 mmol), 342 mL of toluene, 4-bromo-9,9′-spirobi[fluorene] (29.74 g, 1.1 equiv, 75.2 mmol) and P^{t}Bu₃ (1.38 g, 0.1 equiv, 6.8 mmol) were added in the stated order, followed by heating, refluxing and stirring. After cooling to room temperature, water was added to the reaction solution, and an organic layer was separately taken. Toluene was added to an aqueous layer, an organic layer was further extracted, and organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-10 (25.92 g, yield 71%).

By measuring FAB-MS, mass number of m/z=533 was observed as a molecular ion peak, and Intermediate Compound IM-10 was confirmed.

### 4) Synthesis of Compound C22

Under an argon (Ar) atmosphere, to a 300 mL, three-neck flask, Intermediate Compound IM-10 (10.00 g, 18.7 mmol), Pd(dba)₂ (0.32 g, 0.03 equiv, 0.6 mmol), NaO^{t}Bu (3.60 g, 2.0 equiv, 37.5 mmol), 94 mL of toluene, Intermediate Compound IM-9 (7.31 g, 1.1 equiv, 20.6 mmol) and P^{t}Bu₃ (0.38 g, 0.1 equiv, 1.9 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound C22 (11.50 g, yield 72%) as a solid.

By measuring FAB-MS, mass number of m/z=852 was observed as a molecular ion peak, and Compound C22 was confirmed.

### (6) Synthesis of Compound C33

Amine Compound C33 according to an embodiment may be synthesized, for example, by the steps of Reaction 6.

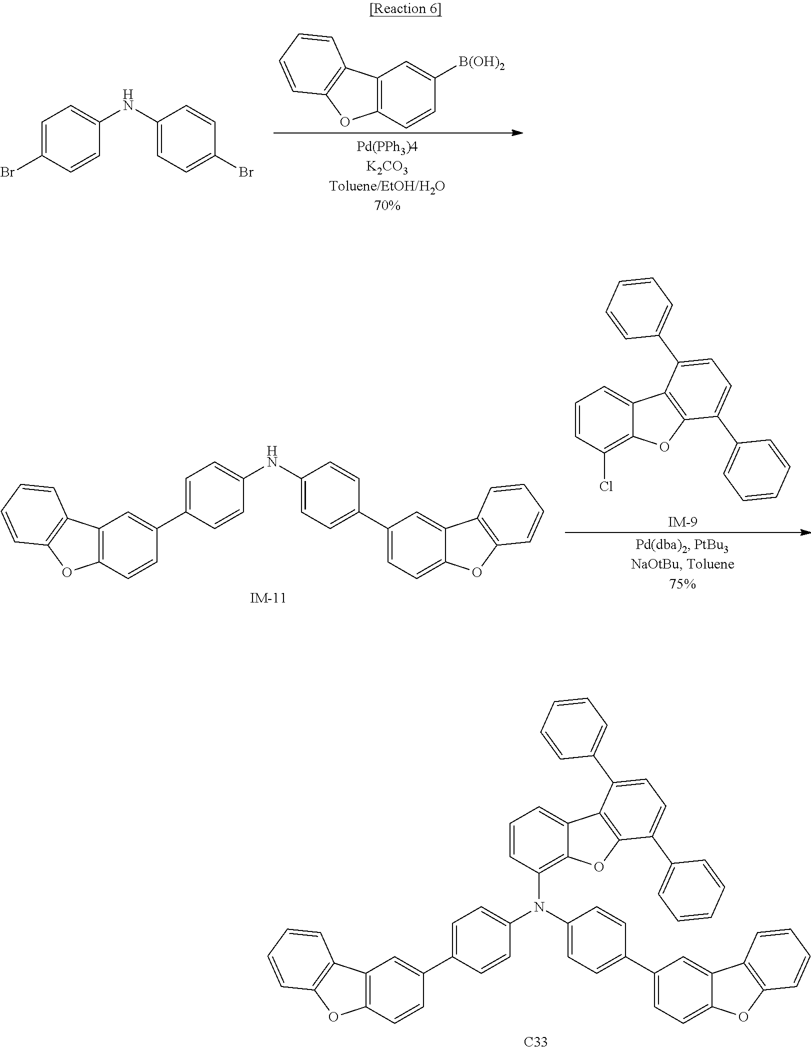

### 1) Synthesis of Intermediate Compound IM-11

Under an argon (Ar) atmosphere, to a 1000 mL, three-neck flask, bis(4-bromophenyl)amine (20.00 g, 61.2 mmol), dibenzofuran-2-ylboronic acid (19.41 g, 2.5 equiv, 152.9 mmol), K₂CO₃ (50.72 g, 6.0 equiv, 367.0 mmol), Pd(PPh₃)₄ (7.07 g, 0.10 eq, 6.1 mmol), and 428 mL of a mixture solution of toluene/EtOH/H₂O (at a volume ratio of 4/2/1) were added in the stated order, followed by heating to about 80° C. and stirring. After cooling to room temperature, the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-11 (21.47 g, yield 70%).

By measuring FAB-MS, mass number of m/z=501 was observed as a molecular ion peak, and Intermediate Compound IM-11 was confirmed.

### 2) Synthesis of Compound C33

Under an argon (Ar) atmosphere, to a 300 mL, three-neck flask, Intermediate Compound IM-11 (10.00 g, 19.9 mmol), Pd(dba)₂ (0.34 g, 0.03 equiv, 0.6 mmol), NaO^{t}Bu (3.83 g, 2.0 equiv, 39.9 mmol), 100 mL of toluene, Intermediate Compound IM-9 (7.78 g, 1.1 equiv, 21.9 mmol) and P^{t}Bu₃ (0.40 g, 0.1 equiv, 2.0 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound C33 (12.26 g, yield 75%) as a solid.

By measuring FAB-MS, mass number of m/z=819 was observed as a molecular ion peak, and Compound C33 was confirmed.

### (7) Synthesis of Compound D4

Amine Compound D4 according to an embodiment may be synthesized, for example, by the steps of Reaction 7.

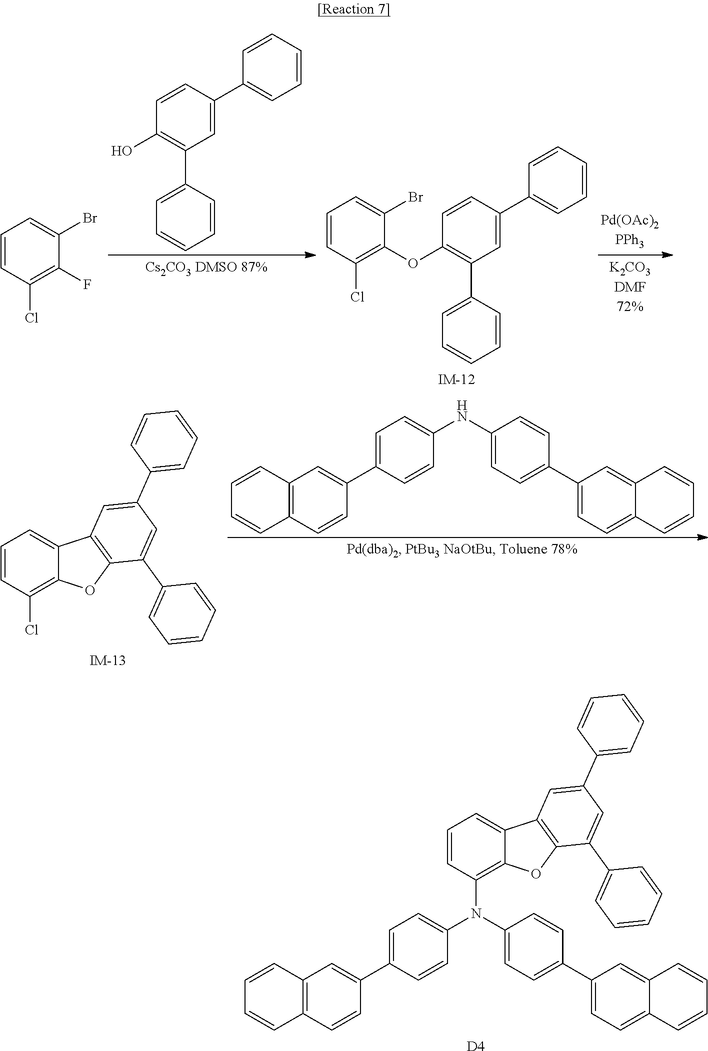

### 1) Synthesis of Intermediate Compound IM-12

Under an argon (Ar) atmosphere, to a 500 mL, three-neck flask, [1,1′:3′,1″-terphenyl]-4′-ol (20.00 g, 81.2 mmol), 1-bromo-3-chloro-2-fluorobenzene (20.41 g, 1.5 equiv, 97.4 mmol), Cs₂CO₃ (52.91 g, 2.0 equiv, 162.4 mmol) and 162 mL of DMSO were added in the stated order, followed by heating to about 100° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-12 (30.78 g, yield 87%).

By measuring FAB-MS, mass number of m/z=435 was observed as a molecular ion peak, and Intermediate Compound IM-12 was confirmed.

### 2) Synthesis of Intermediate Compound IM-13

Under an argon (Ar) atmosphere, to a 500 mL, three-neck flask, Intermediate Compound IM-12 (25.00 g, 57.4 mmol), Pd(OAc)₂ (0.64 g, 0.05 equiv, 2.9 mmol), K₂CO₃ (11.89 g, 1.5 equiv, 86.1 mmol), PPh₃ (1.50 g, 0.10 equiv, 5.7 mmol) and 286 mL of DMF were added in the stated order, followed by heating to about 110° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-13 (14.66 g, yield 72%).

By measuring FAB-MS, mass number of m/z=354 was observed as a molecular ion peak, and Intermediate Compound IM-13 was confirmed.

### 3) Synthesis of Compound D4

Under an argon (Ar) atmosphere, to a 300 mL, three-neck flask, bis[4-(naphthalen-2-yl)phenyl]amine (10.00 g, 23.7 mmol), Pd(dba)₂ (0.41 g, 0.03 equiv, 0.7 mmol), NaO^{t}Bu (4.56 g, 2.0 equiv, 47.4 mmol), 118 mL of toluene, Intermediate Compound IM-13 (9.26 g, 1.1 equiv, 26.1 mmol) and P^{t}Bu₃ (0.48 g, 0.1 equiv, 2.4 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound D4 (13.69 g, yield 78%) as a solid.

By measuring FAB-MS, mass number of m/z=739 was observed as a molecular ion peak, and Compound D4 was confirmed.

### (8) Synthesis of Compound D8

Amine Compound D8 according to an embodiment may be synthesized, for example, by the steps of Reaction 8.

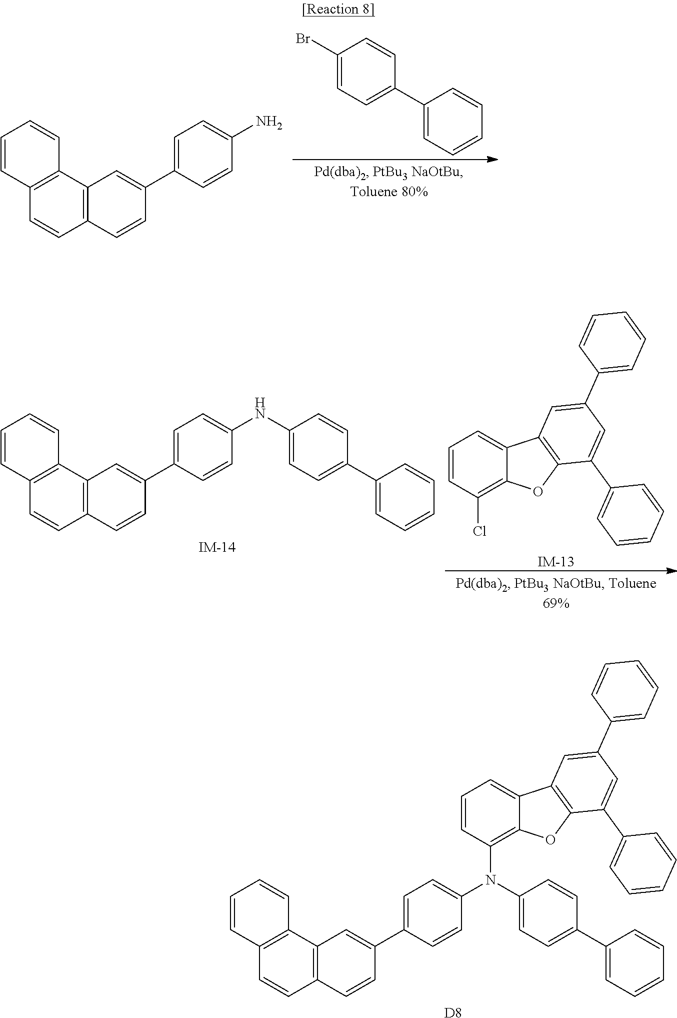

### 1) Synthesis of Intermediate Compound IM-14

Under an argon (Ar) atmosphere, to a 500 mL, three-neck flask, 4-(phenanthren-3-yl)aniline (15.00 g, 55.7 mmol), Pd(dba)₂ (0.96 g, 0.03 equiv, 1.7 mmol), NaO^{t}Bu (5.35 g, 1.0 equiv, 55.7 mmol), 278 mL of toluene, 4-bromobiphenyl (14.28 g, 1.1 equiv, 61.3 mmol) and P^{t}Bu₃ (1.13 g, 0.1 equiv, 5.6 mmol) were added in the stated order, followed by heating, refluxing and stirring. After cooling to room temperature, water was added to the reaction solution, and an organic layer was separately taken. Toluene was added to an aqueous layer, an organic layer was further extracted, and organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-14 (18.78 g, yield 80%).

By measuring FAB-MS, mass number of m/z=421 was observed as a molecular ion peak, and Intermediate Compound IM-14 was confirmed.

### 2) Synthesis of Compound D8

Under an argon (Ar) atmosphere, to a 300 mL, three-neck flask, Intermediate Compound IM-14 (10.00 g, 23.7 mmol), Pd(dba)₂ (0.41 g, 0.03 equiv, 0.7 mmol), NaO^{t}Bu (4.56 g, 2.0 equiv, 47.4 mmol), 118 mL of toluene, Intermediate Compound IM-13 (11.37 g, 1.1 equiv, 26.1 mmol) and P^{t}Bu₃ (0.48 g, 0.1 equiv, 2.4 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound D8 (12.11 g, yield 69%) as a solid.

By measuring FAB-MS, mass number of m/z=739 was observed as a molecular ion peak, and Compound D8 was confirmed.

### (9) Synthesis of Compound D18

Amine Compound D18 according to an embodiment may be synthesized, for example, by the steps of Reaction 9.

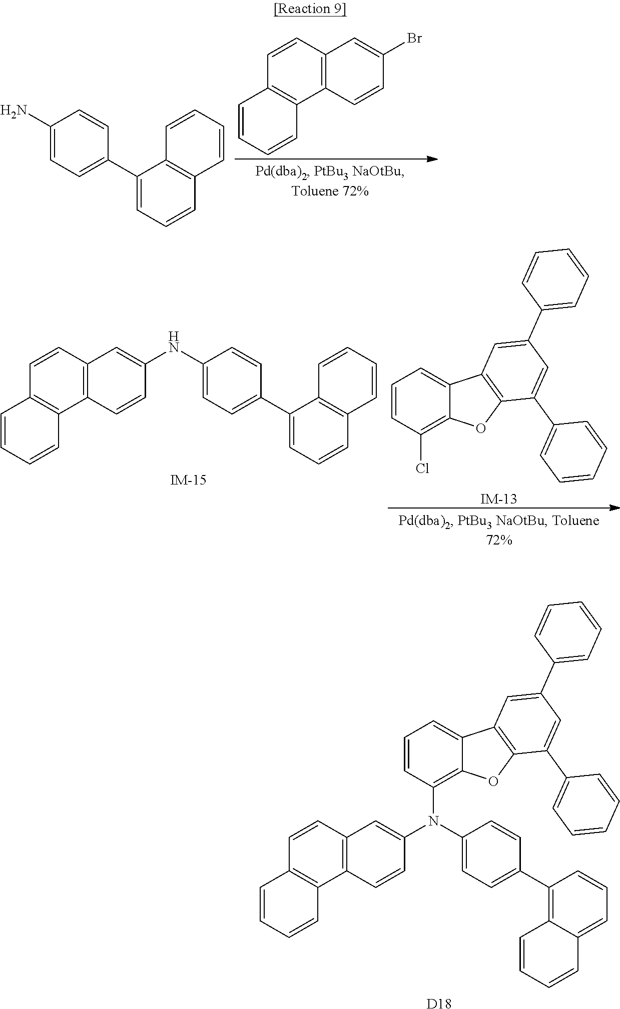

### 1) Synthesis of Intermediate Compound IM-15

Under an argon (Ar) atmosphere, to a 500 mL, three-neck flask, 4-(naphthalen-1-yl)aniline (15.00 g, 68.4 mmol), Pd(dba)₂ (1.18 g, 0.03 equiv, 2.1 mmol), NaO^{t}Bu (6.57 g, 1.0 equiv, 68.4 mmol), 342 mL of toluene, 2-bromophenanthrene (19.35 g, 1.1 equiv, 75.2 mmol) and P^{t}Bu₃ (1.38 g, 0.1 equiv, 6.8 mmol) were added in the stated order, followed by heating, refluxing and stirring. After cooling to room temperature, water was added to the reaction solution, and an organic layer was separately taken. Toluene was added to an aqueous layer, an organic layer was further extracted, and organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-15 (19.48 g, yield 72%).

By measuring FAB-MS, mass number of m/z=395 was observed as a molecular ion peak, and Intermediate Compound IM-15 was confirmed.

### 2) Synthesis of Compound D18

Under an argon (Ar) atmosphere, to a 300 mL, three-neck flask, Intermediate Compound IM-15 (10.00 g, 25.3 mmol), Pd(dba)₂ (0.44 g, 0.03 equiv, 0.8 mmol), NaO^{t}Bu (4.86 g, 2.0 equiv, 50.7 mmol), 126 mL of toluene, Intermediate Compound IM-13 (9.87 g, 1.1 equiv, 27.8 mmol) and P^{t}Bu₃ (0.51 g, 0.1 equiv, 2.5 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound D18 (13.00 g, yield 72%) as a solid.

By measuring FAB-MS, mass number of m/z=713 was observed as a molecular ion peak, and Compound D18 was confirmed.

### (10) Synthesis of Compound D67

Amine Compound D67 according to an embodiment may be synthesized, for example, by the steps of Reaction 10.

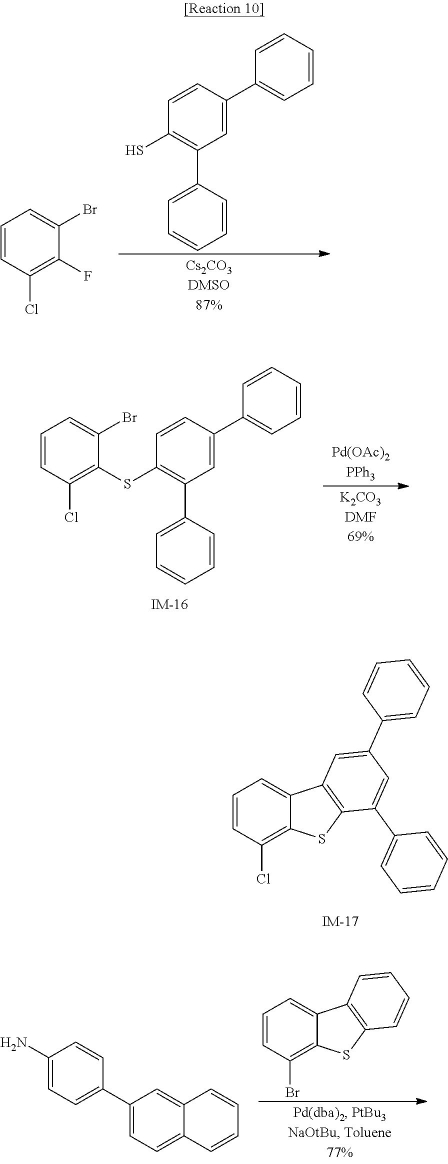

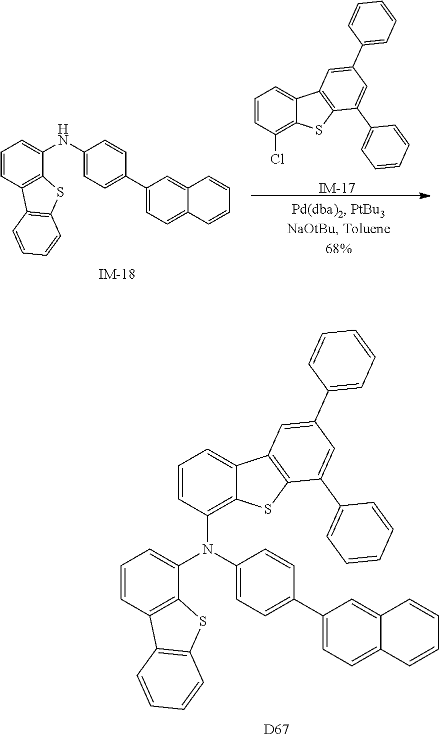

### 1) Synthesis of Intermediate Compound IM-16

Under an argon (Ar) atmosphere, to a 500 mL, three-neck flask, [1,1′:3′,1″-terphenyl]-4′-thiol (20.00 g, 76.2 mmol), 1-bromo-3-chloro-2-fluorobenzene (19.16 g, 1.5 equiv, 91.5 mmol), Cs₂CO₃ (49.67 g, 2.0 equiv, 152.5 mmol) and 152 mL of DMSO were added in the stated order, followed by heating to about 100° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-16 (29.96 g, yield 87%).

By measuring FAB-MS, mass number of m/z=451 was observed as a molecular ion peak, and Intermediate Compound IM-16 was confirmed.

### 2) Synthesis of Intermediate Compound IM-17

Under an argon (Ar) atmosphere, to a 500 mL, three-neck flask, Intermediate Compound IM-16 (25.00 g, 55.3 mmol), Pd(OAc)₂ (0.62 g, 0.05 equiv, 2.8 mmol), K₂CO₃ (11.47 g, 1.5 equiv, 83.0 mmol), PPh₃ (1.45 g, 0.10 equiv, 5.5 mmol) and 277 mL of DMF were added in the stated order, followed by heating to about 110° C. and stirring. After cooling to room temperature, water was added, and the reaction solution was extracted with toluene. An aqueous layer was removed, and an organic layer was washed with a saturated saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-17 (14.16 g, yield 69%).

By measuring FAB-MS, mass number of m/z=370 was observed as a molecular ion peak, and Intermediate Compound IM-17 was confirmed.

### 3) Synthesis of Intermediate Compound IM-18

Under an argon (Ar) atmosphere, to a 1000 mL, three-neck flask, 4-(naphthalen-2-yl)aniline (15.00 g, 68.4 mmol), Pd(dba)₂ (1.18 g, 0.03 equiv, 2.1 mmol), NaO^{t}Bu (6.57 g, 1.0 equiv, 68.4 mmol), 342 mL of toluene, 4-bromodibenzothiophene (19.80 g, 1.1 equiv, 75.2 mmol) and P^{t}Bu₃ (1.38 g, 0.1 equiv, 6.8 mmol) were added in the stated order, followed by heating, refluxing and stirring. After cooling to room temperature, water was added to the reaction solution, and an organic layer was separately taken. Toluene was added to an aqueous layer, an organic layer was further extracted, and organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Intermediate Compound IM-18 (21.15 g, yield 77%).

By measuring FAB-MS, mass number of m/z=401 was observed as a molecular ion peak, and Intermediate Compound IM-18 was confirmed.

### 4) Synthesis of Compound D67

Under an argon (Ar) atmosphere, to a 300 mL, three-neck flask, Intermediate Compound IM-18 (10.00 g, 24.9 mmol), Pd(dba)₂ (0.43 g, 0.03 equiv, 0.7 mmol), NaO^{t}Bu (4.79 g, 2.0 equiv, 49.8 mmol), 125 mL of toluene, Intermediate Compound IM-17 (10.16 g, 1.1 equiv, 27.4 mmol) and P^{t}Bu₃ (0.50 g, 0.1 equiv, 2.5 mmol) were added in the stated order, followed by heating, refluxing and stirring. After cooling to room temperature, water was added to the reaction solution, and an organic layer was separately taken. Toluene was added to an aqueous layer, an organic layer was further extracted, and the organic layers were collected, washed with a saline solution and dried over MgSO₄. MgSO₄ was filtered, an organic layer was concentrated, and the crude product thus obtained was separated by silica gel column chromatography (a mixture solvent of hexane and toluene was utilized as an eluent) to obtain Compound D67 (12.46 g, yield 68%) as a solid.

By measuring FAB-MS, mass number of m/z=735 was observed as a molecular ion peak, and Compound D67 was confirmed.

### 2. Manufacture and Evaluation of Light Emitting Elements

The evaluation on the light emitting elements including the Example Compounds and Comparative Compounds in hole transport layers were conducted by methods described below. The manufacturing method of the light emitting elements for evaluating the elements is described below.

### (1) Manufacture of Light Emitting Element 1

On a glass substrate, ITO with a thickness of about 1500 Å was patterned, washed with ultrapure water and treated with UV ozone for about 10 minutes to form a first electrode. Then, 2-TNATA was deposited on the first electrode to a thickness of about 600 Å to form a hole injection layer. After that, an Example Compound or a Comparative Compound was deposited on the hole injection layer to a thickness of about 300 Å to form a hole transport layer.

Then, an emission layer with a thickness of about 250 Å was formed on the hole transport layer utilizing ADN doped with 3% TBP. Alq₃ was deposited on the emission layer to a thickness of about 250 Å to form an electron transport layer, and LiF was deposited on the electron transport layer to a thickness of about 10 Å to form an electron injection layer.

After that, a second electrode was formed on the electron injection layer by providing aluminum (Al) to a thickness of about 1000 Å.

In the Examples, the hole injection layer, the hole transport layer, the emission layer, the electron transport layer, the electron injection layer and the second electrode were each formed utilizing a vacuum deposition apparatus.

### (2) Manufacture of Light Emitting Element 2

On a glass substrate, ITO with a thickness of about 1500 Å was patterned, washed with ultrapure water and treated with UV ozone for about 10 minutes to form a first electrode. Then, 2-TNATA was deposited on the first electrode to a thickness of about 600 Å to form a hole injection layer. After that, H-1-1 was deposited on the hole injection layer to a thickness of about 200 Å to form a hole transport layer, and an Example Compound or a Comparative Compound was deposited on the hole transport layer to a thickness of about 100 Å to form an electron blocking layer.

Then, an emission layer with a thickness of about 250 Å was formed on the electron blocking layer utilizing ADN doped with 3% TBP. Alq₃ was deposited on the emission layer to a thickness of about 250 Å to form an electron transport layer, and LiF was deposited on the electron transport layer to a thickness of about 10 Å to form an electron injection layer.

After that, a second electrode was formed on the electron injection layer by providing aluminum (Al) to a thickness of about 1000 Å.

In the Examples, the hole injection layer, the hole transport layer, the electron blocking layer, the emission layer, the electron transport layer, the electron injection layer and the second electrode were each formed utilizing a vacuum deposition apparatus.

Meanwhile, the molecular weights of the Example Compounds were measured by FAB-MS utilizing JMS-700V of JEOL Co. In addition, NMR of the Example Compounds was obtained by measuring 1H-NMR utilizing AVAVCE300M of Bruker Biospin K.K. In the evaluation results of the light emitting elements, the current density, voltage and emission efficiency of the elements were measured utilizing Source Meter of a 2400 Series product of Keithley Instruments Co., a luminance and color meter of CS-200 of Konica Minolta Co., Ltd., and a product of PC Program LabVIEW8.2 for measurement of Japanese National Instruments Co., in a dark room.

The Example Compounds and Comparative Compounds utilized for the manufacture of light emitting element 1 and light emitting element 2 are as follows.

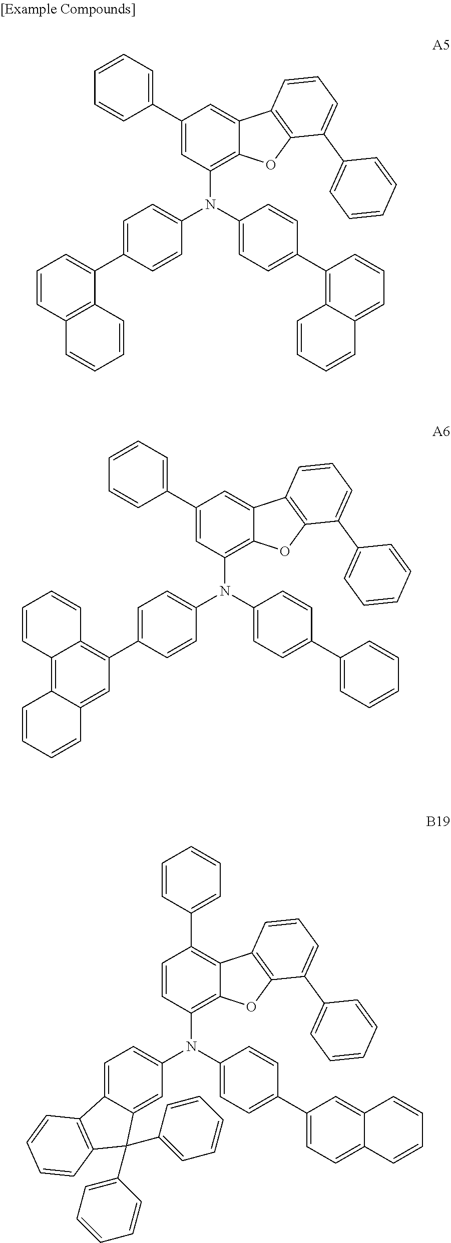 [SMILES: c1ccc(-c2cc(N(c3ccc(-c4cccc5ccccc45)cc3)c3ccc(-c4cccc5ccccc45)cc3)c3oc4c(-c5ccccc5)cccc4c3c2)cc1 ; c1ccc(-c2ccc(N(c3ccc(-c4cc5ccccc5c5ccccc45)cc3)c3cc(-c4ccccc4)cc4c3oc3c(-c5ccccc5)cccc34)cc2)cc1 ; c1ccc(-c2cccc3c2oc2c(N(c4ccc(-c5ccc6ccccc6c5)cc4)c4ccc5c(c4)C(c4ccccc4)(c4ccccc4)c4ccccc4-5)ccc(-c4ccccc4)c23)cc1]

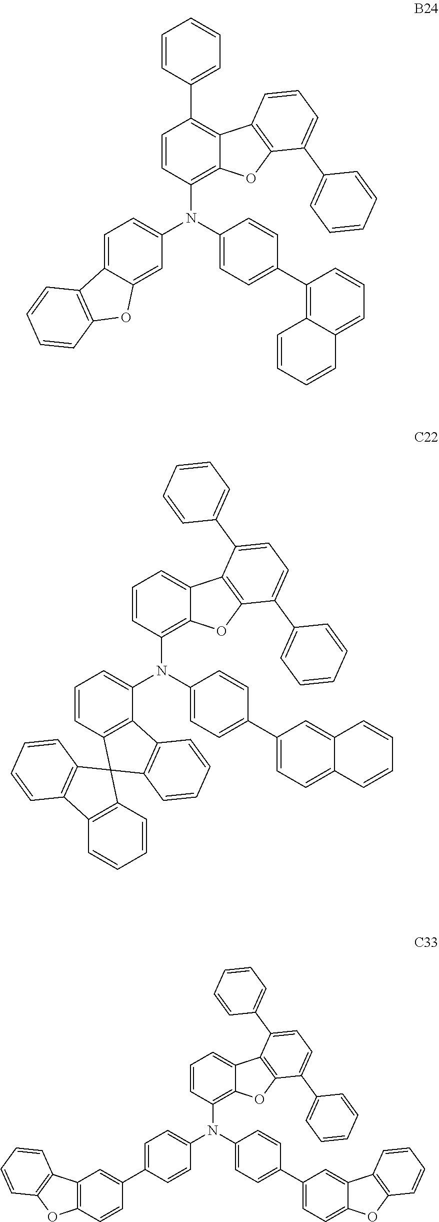 [SMILES: c1ccc(-c2cccc3c2oc2c(N(c4ccc(-c5cccc6ccccc56)cc4)c4ccc5c(c4)oc4ccccc45)ccc(-c4ccccc4)c23)cc1 ; c1ccc(-c2ccc(-c3ccccc3)c3c2oc2c(N(c4ccc(-c5ccc6ccccc6c5)cc4)c4cccc5c4-c4ccccc4C54c5ccccc5-c5ccccc54)cccc23)cc1 ; c1ccc(-c2ccc(-c3ccccc3)c3c2oc2c(N(c4ccc(-c5ccc6oc7ccccc7c6c5)cc4)c4ccc(-c5ccc6oc7ccccc7c6c5)cc4)cccc23)cc1]

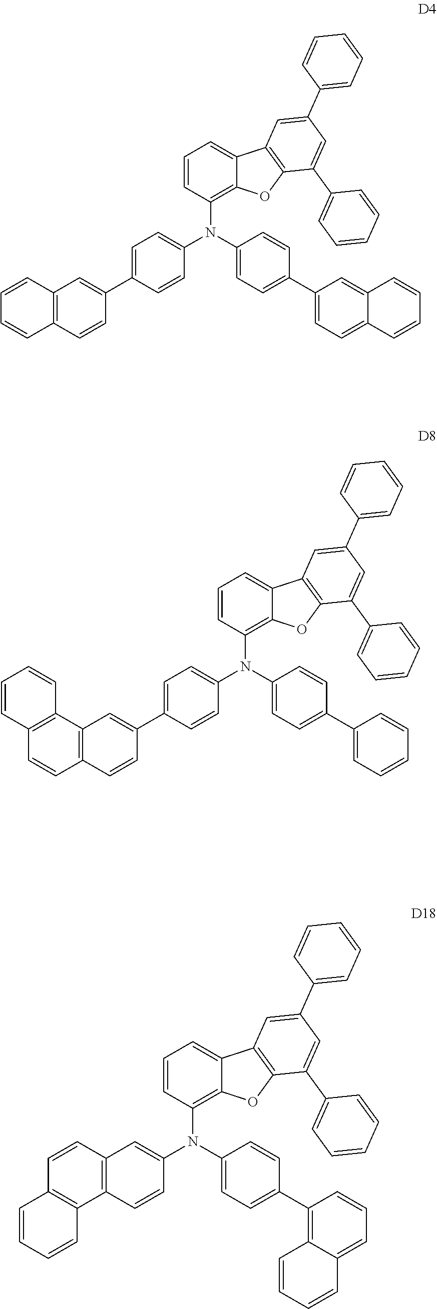 [SMILES: c1ccc(-c2cc(-c3ccccc3)c3oc4c(N(c5ccc(-c6ccc7ccccc7c6)cc5)c5ccc(-c6ccc7ccccc7c6)cc5)cccc4c3c2)cc1 ; c1ccc(-c2ccc(N(c3ccc(-c4ccc5ccc6ccccc6c5c4)cc3)c3cccc4c3oc3c(-c5ccccc5)cc(-c5ccccc5)cc34)cc2)cc1 ; c1ccc(-c2cc(-c3ccccc3)c3oc4c(N(c5ccc(-c6cccc7ccccc67)cc5)c5ccc6c(ccc7ccccc76)c5)cccc4c3c2)cc1]

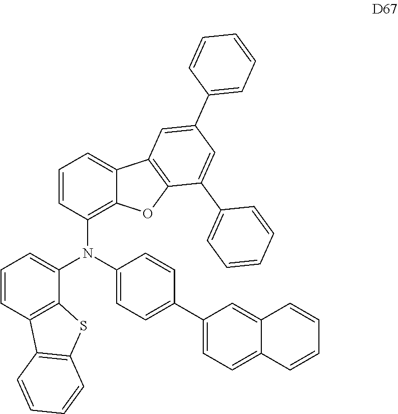 [SMILES: c1ccc(-c2cc(-c3ccccc3)c3oc4c(N(c5ccc(-c6ccc7ccccc7c6)cc5)c5cccc6c5sc5ccccc56)cccc4c3c2)cc1]

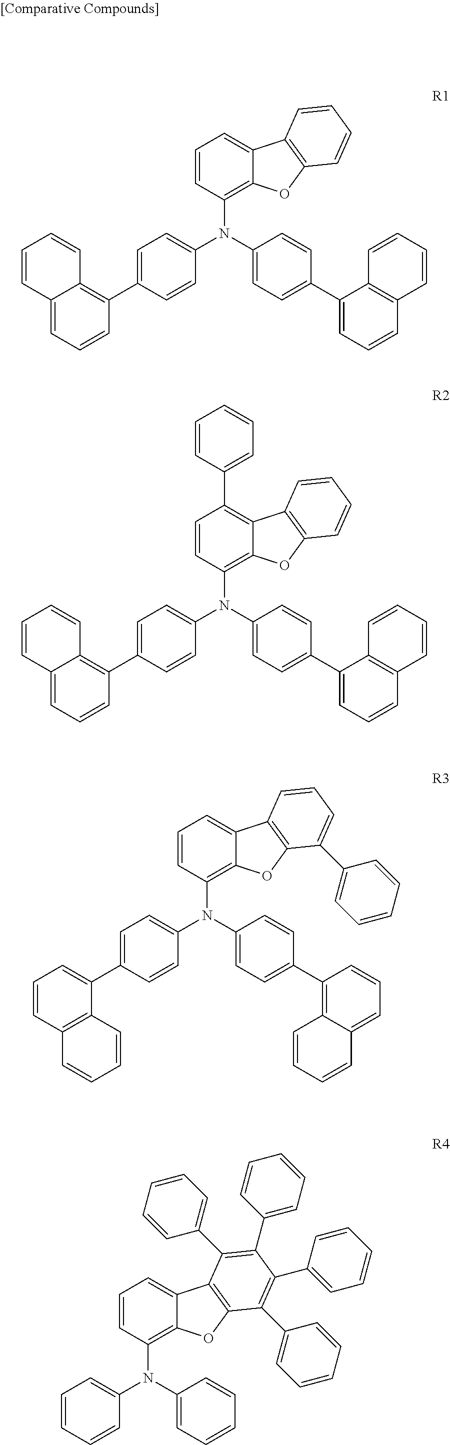 [SMILES: c1ccc2c(-c3ccc(N(c4ccc(-c5cccc6ccccc56)cc4)c4cccc5c4oc4ccccc45)cc3)cccc2c1 ; c1ccc(-c2ccc(N(c3ccc(-c4cccc5ccccc45)cc3)c3ccc(-c4cccc5ccccc45)cc3)c3oc4ccccc4c23)cc1 ; c1ccc(-c2cccc3c2oc2c(N(c4ccc(-c5cccc6ccccc56)cc4)c4ccc(-c5cccc6ccccc56)cc4)cccc23)cc1 ; c1ccc(-c2c(-c3ccccc3)c(-c3ccccc3)c3c(oc4c(N(c5ccccc5)c5ccccc5)cccc43)c2-c2ccccc2)cc1]

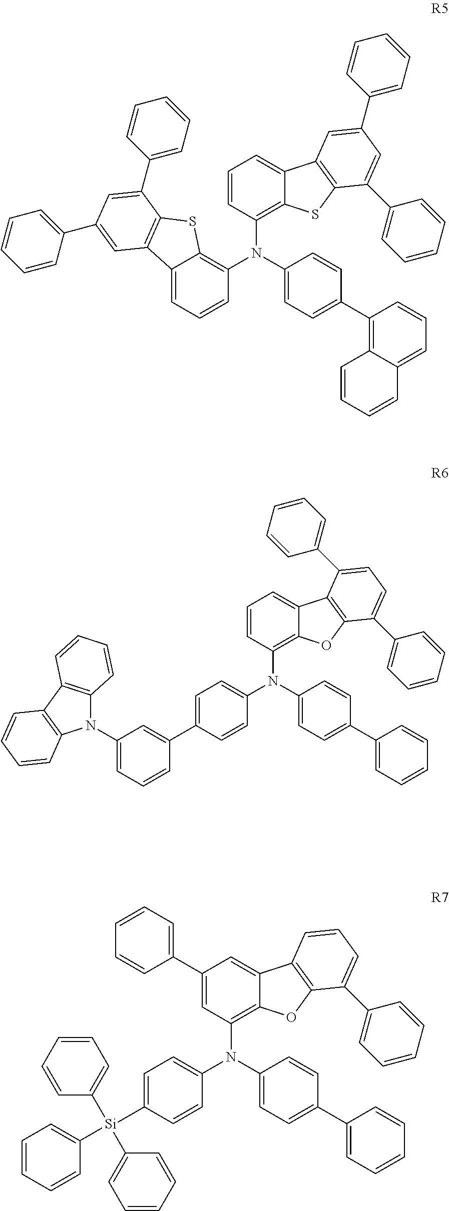 [SMILES: c1ccc(-c2cc(-c3ccccc3)c3sc4c(N(c5ccc(-c6cccc7ccccc67)cc5)c5cccc6c5sc5c(-c7ccccc7)cc(-c7ccccc7)cc56)cccc4c3c2)cc1 ; c1ccc(-c2ccc(N(c3ccc(-c4cccc(-n5c6ccccc6c6ccccc65)c4)cc3)c3cccc4c3oc3c(-c5ccccc5)ccc(-c5ccccc5)c34)cc2)cc1 ; c1ccc(-c2ccc(N(c3ccc([Si](c4ccccc4)(c4ccccc4)c4ccccc4)cc3)c3cc(-c4ccccc4)cc4c3oc3c(-c5ccccc5)cccc34)cc2)cc1]

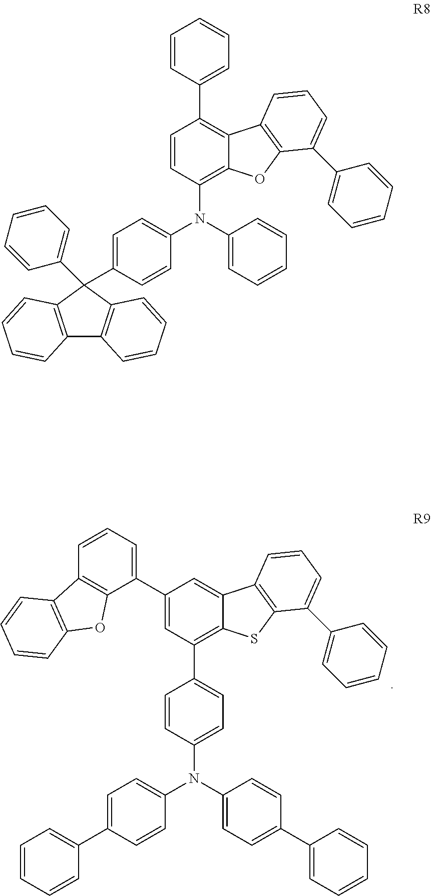 [SMILES: c1ccc(-c2cccc3c2oc2c(N(c4ccccc4)c4ccc(C5(c6ccccc6)c6ccccc6-c6ccccc65)cc4)ccc(-c4ccccc4)c23)cc1 ; c1ccc(-c2ccc(N(c3ccc(-c4ccccc4)cc3)c3ccc(-c4cc(-c5cccc6c5oc5ccccc56)cc5c4sc4c(-c6ccccc6)cccc45)cc3)cc2)cc1]

Besides, the compounds of the functional layers, utilized for the manufacture of light emitting elements 1 and 2 are as follows.

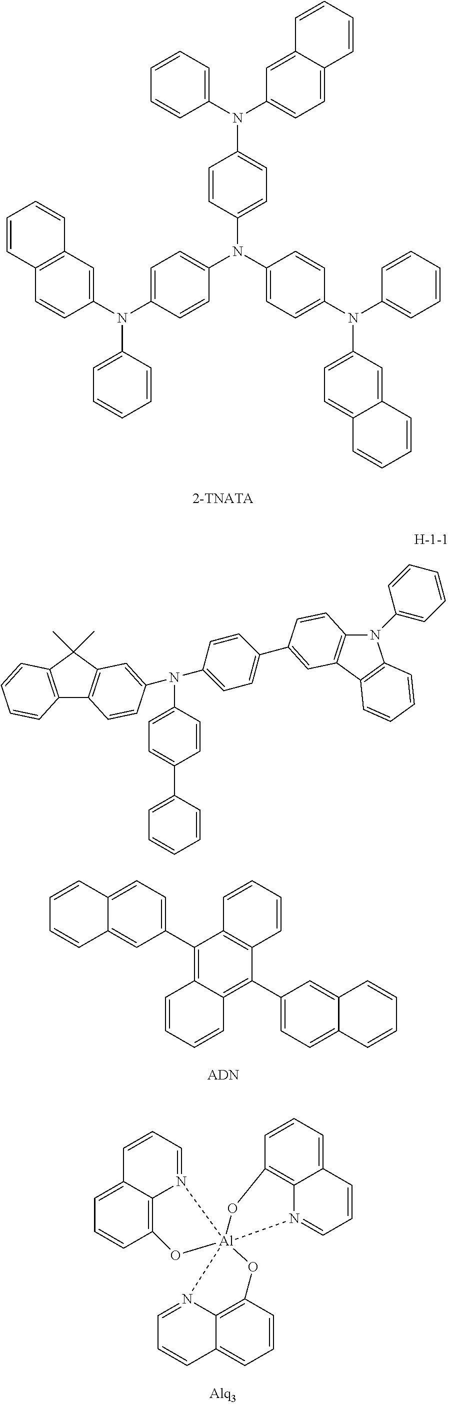

### (2) Evaluation of Light Emitting Elements 1 and Light Emitting Elements 2

### 1) Evaluation of Light Emitting Elements 1

Table 1 shows evaluation results on the light emitting elements 1 of Examples 1-1 to 1-10, and Comparative Examples 1-1 to 1-9, and Table 2 shows evaluation results on the light emitting elements 2 of Examples 2-1 to 2-10, and Comparative Examples 2-1 to 2-9. In Table 1 and Table 2, the maximum emission efficiency and half life of the light emitting elements 1 and light emitting elements 2 manufactured are compared and shown. In the evaluation results of the properties of the Examples and Comparative Examples, shown in Tables 1 and 2, the emission efficiency shows the efficiency values at a current density of about 10 mA/cm².

The element life shows the time when the luminance was 50% from initial luminance while continuously driving at about 1000 cd/m², as relative values compared to Comparative Example 1-1.

In Table 1 and Table 2, the emission efficiency and element life are relative values utilizing the emission efficiency and life of Comparative Example 1-1 and the emission efficiency and life of Comparative Example 2-1 as being 100%. That is, the emission efficiency and element life shown in Table 1 are relative values utilizing the emission efficiency and element life of Comparative Example 1-1 as being 100%, each, and the emission efficiency and element life shown in Table 2 are relative values utilizing the emission efficiency and the element life of Comparative Example 2-1 as being 100%, each.

TABLE 1

| Element | | Emission | Element |
|---|---|---|---|
| manufacturing | | efficiency | life |
| example | Hole transport layer | @10 mA/cm² | LT50 |
| Example 1-1 | Example Compound A5 | 147% | 175% |
| Example 1-2 | Example Compound A6 | 146% | 173% |
| Example 1-3 | Example Compound B19 | 143% | 185% |
| Example 1-4 | Example Compound B24 | 140% | 180% |
| Example 1-5 | Example Compound C22 | 150% | 170% |
| Example 1-6 | Example Compound C33 | 143% | 185% |
| Example 1-7 | Example Compound D4 | 142% | 190% |
| Example 1-8 | Example Compound D8 | 139% | 186% |
| Example 1-9 | Example Compound D18 | 142% | 182% |
| Example 1-10 | Example Compound D67 | 145% | 178% |
| Comparative | Comparative Compound | 100% | 100% |
| Example 1-1 | R1 | | |
| Comparative | Comparative Compound | 109% | 91% |
| Example 1-2 | R2 | | |
| Comparative | Comparative Compound | 97% | 115% |
| Example 1-3 | R3 | | |
| Comparative | Comparative Compound | 88% | 74% |
| Example 1-4 | R4 | | |
| Comparative | Comparative Compound | 95% | 92% |
| Example 1-5 | R5 | | |
| Comparative | Comparative Compound | 114% | 118% |
| Example 1-6 | R6 | | |
| Comparative | Comparative Compound | 103% | 115% |
| Example 1-7 | R7 | | |
| Comparative | Comparative Compound | 90% | 99% |
| Example 1-8 | R8 | | |
| Comparative | Comparative Compound | 97% | 103% |
| Example 1-9 | R9 | | |

Referring to the results of Table 1, it could be found that the Examples of the light emitting elements, utilizing the amine compounds of embodiments of the present disclosure as the materials of hole transport layers showed excellent or suitable emission efficiency and improved element-life characteristics. That is, the amine compounds of the Examples have a dibenzoheterole group substituted with two aryl groups, and showed relatively high efficiency and long-life characteristics at the same time when compared to the Comparative Examples.

Particularly, in the amine compound of an embodiment, an aryl group is substituted at position 6 of a dibenzoheterole group. In the amine compound of an embodiment, the aryl group substituted at position 6 of the dibenzoheterole group is oriented as if to cover (e.g., close to) the heteroatom of the dibenzoheterole ring to contribute to the stabilization of a radical or radical cation state. In addition to position 6 of the dibenzoheterole group, an aryl group oriented as if to spread out (e.g., to extend the size) of a molecule is further substituted in the amine compound of an embodiment. Accordingly, the amine compound of an embodiment increases intermolecular interaction, improves hole transport capacity and may contribute to the decrease of the driving voltage and increase of the efficiency of the light emitting element. Accordingly, it can be seen that light emitting elements of Examples 1-1 to 1-10, each utilizing the amine compounds of the Examples, showed high efficiency and long-life characteristics.

In comparison, the Comparative Compounds utilized in Comparative Examples 1-1 to 1-3 are materials with fewer (e.g., 0 or 1) aryl groups substituted on a dibenzoheterole ring, and the stabilization effects and improving effects of hole transport capacity by an aryl group, as shown in the amine compound of an embodiment, are reduced. Accordingly, both (e.g., simultaneously) the emission efficiency and element life of Comparative Examples 1-1 to 1-3 were degraded when compared to the Examples.

Comparative Compound R4 utilized in Comparative Example 1-4 is a material in which four phenyl groups are substituted at a dibenzoheterole ring, and Comparative Compound R5 utilized in Comparative Example 1-5 is an amine compound having two dibenzothiophene groups at which two aryl groups are substituted. Comparative Examples 1-4 and 1-5, utilizing Comparative Compound R4 and Comparative Compound R5, respectively, each showed degraded emission efficiency and element life when compared to the Examples. It is thought that when phenyl groups are excessively substituted, the deposition temperature of the materials was elevated, and the deterioration of materials occurred under high temperature conditions.

Comparative Compound R6 utilized in Comparative Example 1-6 is a material having a carbazole group in a molecule, and carrier balance was collapsed, and both emission efficiency and life were deteriorated.

Comparative Example 1-7 utilized Comparative Compound R7, having a silyl group in a molecule, and due to the influence of sterically large silyl group, intermolecular interaction was reduced, and emission efficiency was particularly reduced when compared to the Examples.

Comparative Compound R8 utilized in Comparative Example 1-8 is a material having a 9-fluorene group in a molecule. Comparative Example 1-8 utilizing Comparative Compound R8 showed degraded emission efficiency and element life when compared to the Examples. In the case where an amine moiety was extended at (e.g., connected through) position 9 of the fluorene as in Comparative Compound R9, the radical or radical cation state of the compound was destabilized, bond cleavage occurred around a sp3 carbon atom, and the material was deteriorated. In contrast, in the cases where an amine moiety was extended at (e.g., connected through) a fluorene ring skeleton side as in Example Compounds B19 and C22, the compounds were stabilized even in a radical or radical cation state. Accordingly, the light emitting element of an embodiment may show excellent or suitable element characteristics.

Comparative Compound R9 utilized in Comparative Example 1-9 is a material in which a dibenzothiophene group to which a dibenzofuranyl group is bonded is combined with a nitrogen atom via a phenylene linker, and carrier balance was collapsed and the deposition temperature of the material was also increased to deteriorate the material. Accordingly, Comparative Example 1-9 utilizing Comparative Compound R9 showed degraded emission efficiency and element life when compared to the Examples.

TABLE 2

| Element | | Emission | Element |
|---|---|---|---|
| manufacturing | | efficiency | life |
| example | Electron blocking layer | @ 10 mA/cm² | LT50 |
| Example 2-1 | Example Compound A5 | 145% | 170% |
| Example 2-2 | Example Compound A6 | 141% | 168% |
| Example 2-3 | Example Compound B19 | 143% | 180% |
| Example 2-4 | Example Compound B24 | 138% | 178% |
| Example 2-5 | Example Compound C22 | 145% | 165% |
| Example 2-6 | Example Compound C33 | 140% | 179% |
| Example 2-7 | Example Compound D4 | 144% | 195% |
| Example 2-8 | Example Compound D8 | 135% | 188% |
| Example 2-9 | Example Compound D18 | 139% | 174% |
| Example 2-10 | Example Compound D67 | 140% | 170% |
| Comparative | Comparative Compound | 100% | 100% |
| Example 2-1 | R1 | | |
| Comparative | Comparative Compound | 108% | 97% |
| Example 2-2 | R2 | | |
| Comparative | Comparative Compound | 97% | 108% |
| Example 2-3 | R3 | | |
| Comparative | Comparative Compound | 89% | 74% |
| Example 2-4 | R4 | | |
| Comparative | Comparative Compound | 93% | 92% |
| Example 2-5 | R5 | | |
| Comparative | Comparative Compound | 111% | 117% |
| Example 2-6 | R6 | | |
| Comparative | Comparative Compound | 102% | 109% |
| Example 2-7 | R7 | | |
| Comparative | Comparative Compound | 88% | 98% |
| Example 2-8 | R8 | | |
| Comparative | Comparative Compound | 93% | 105% |
| Example 2-9 | R9 | | |

Referring to the results of Table 2, it could be confirmed that Example 2-1 to Example 2-10 each showed long-life and high efficiency characteristics when compared to the light emitting elements of Comparative Example 2-1 to Comparative Example 2-9. For example, it could be found that excellent or suitable element characteristics could be shown even in the case of utilizing the amine compound of an embodiment in an electron blocking layer.

As described above, each of the compounds utilized in the Examples may improve emission efficiency and emission life at the same time when compared to the compounds utilized in the Comparative Examples. That is, by utilizing an amine compound including a dibenzoheterole group at which two aryl groups are substituted in the light emitting element of an embodiment, element efficiency and element life may be improved at the same time.

The light emitting element of an embodiment includes an amine compound of an embodiment in a hole transport region and may show high efficiency and long-life characteristics.

The amine compound of an embodiment may be utilized as a material for accomplishing improved properties of a light emitting element, with high efficiency and long life.

As used herein, expressions such as “at least one of,” “one of,” and “selected from,” when preceding a list of elements, modify the entire list of elements and do not modify the individual elements of the list.

As used herein, the term “and/or” includes any and all combinations of one or more of the associated listed items. Further, the use of “may” when describing embodiments of the present disclosure refers to “one or more embodiments of the present disclosure”.

As used herein, the terms “substantially,” “about,” and similar terms are used as terms of approximation and not as terms of degree, and are intended to account for the inherent deviations in measured or calculated values that would be recognized by those of ordinary skill in the art. Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The display device and/or any other relevant devices or components according to embodiments of the present invention described herein may be implemented utilizing any suitable hardware, firmware (e.g. an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments.

Although the embodiments of the present disclosure have been described, it is understood that the present disclosure should not be limited to these embodiments, but various suitable changes and modifications can be made by one ordinary skilled in the art within the spirit and scope of the present disclosure as hereinafter claimed, and equivalents thereof.

What is claimed is:

1. A light emitting element, comprising:
a first electrode;
a second electrode on the first electrode; and
at least one functional layer between the first electrode and the second electrode, and comprising an amine compound represented by Formula 1: 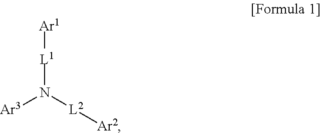
wherein in Formula 1,
L¹ and L² are each independently a direct linkage, a substituted or unsubstituted arylene group having 6 to 30 ring-forming carbon atoms, or a substituted or unsubstituted heteroarylene group having 5 to 30 ring-forming carbon atoms,
Ar¹ and Ar² are each independently a substituted or unsubstituted aryl group having 6 to 30 ring-forming carbon atoms, or a substituted or unsubstituted heteroaryl group having 5 to 30 ring-forming carbon atoms,
in case where Ar¹ or Ar² is a substituted or unsubstituted fluorenyl group, any one selected from among two benzene rings forming the 3-ring fluorenyl group is bonded with L¹ and L², or bonded with the nitrogen atom of Formula 1,
L¹-Ar¹ and L²-Ar² do not comprise a carbazole group or a silyl group, and
Ar³ is different from Ar¹ and Ar², and is represented by Formula 2: 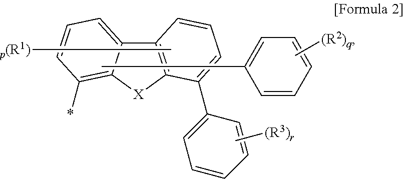
wherein in Formula 2,
X is O or S,
R¹ is a hydrogen atom, a deuterium atom, a halogen atom, or a substituted or unsubstituted alkyl group having 1 to 20 carbon atoms,
R² and R³ are each independently a hydrogen atom, a deuterium atom, a halogen atom, a substituted or unsubstituted alkyl group having 1 to 20 carbon atoms, or a substituted or unsubstituted aryl group having 6 to 30 ring-forming carbon atoms, or combined with an adjacent group to form a hydrocarbon ring,
“p”, “q” and “r” are each independently an integer of 0 to 5, and
*- is a position combined with the nitrogen atom of Formula 1.

2. The light emitting element of claim 1, wherein
the at least one functional layer comprises an emission layer, a hole transport region between the first electrode and the emission layer, and an electron transport region between the emission layer and the second electrode, and
the hole transport region comprises the amine compound.

3. The light emitting element of claim 2, wherein
the hole transport region comprises a hole injection layer, and a hole transport layer on the hole injection layer, and
the hole transport layer comprises the amine compound.

4. The light emitting element of claim 2, wherein
the hole transport region comprises a hole injection layer, a hole transport layer, and an electron blocking layer, stacked in the stated order, and
the electron blocking layer comprises the amine compound.

5. The light emitting element of claim 1, wherein Formula 2 is represented by any one selected from among Formula 2-1 to Formula 2-4: 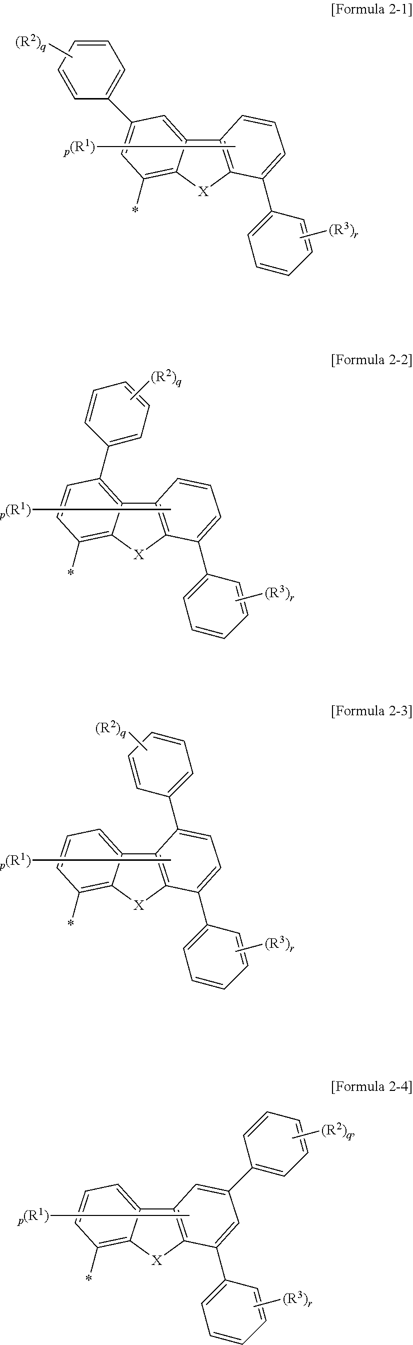
wherein in Formula 2-1 to Formula 2-4, X, R¹, R², R³, “p”, “q” and “r” are the same as respectively defined in connection with Formula 2.

6. The light emitting element of claim 1, wherein the amine compound is represented by Formula 3: 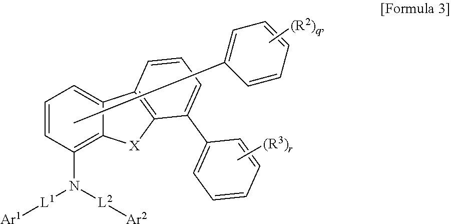
wherein in Formula 3, X, L¹, L², Ar¹, Ar², R², R³, “q” and “r” are the same as respectively defined in connection with Formula 1 and Formula 2.

7. The light emitting element of claim 6, wherein Formula 3 is represented by any one selected from among Formula 3-1 to Formula 3-4: 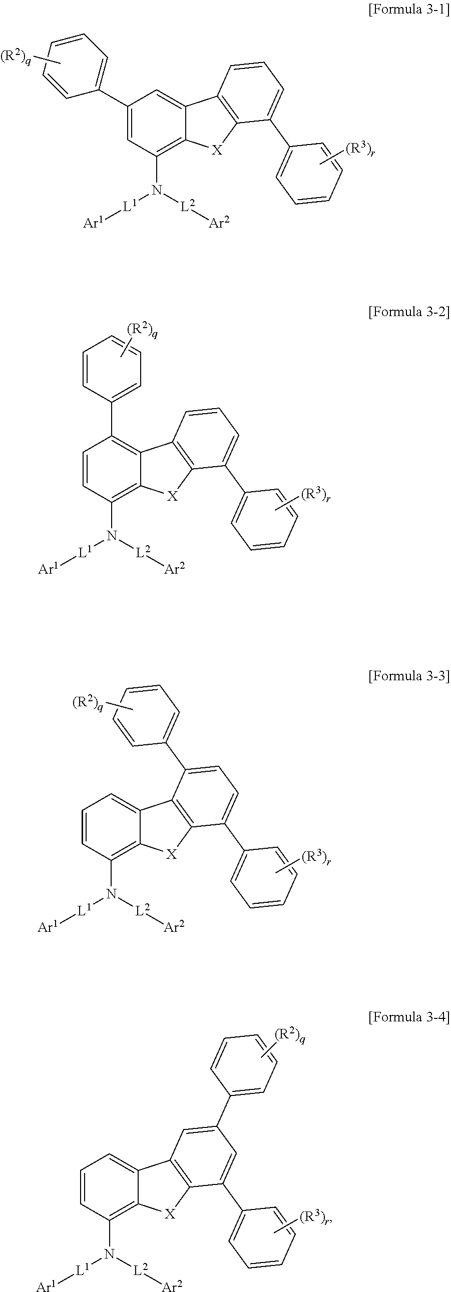
wherein in Formula 3-1 to Formula 3-4, X, L¹, L², Ar¹, Ar², R², R³, “q” and “r” are the same as respectively defined in connection with Formula 1 and Formula 2.

8. The light emitting element of claim 1, wherein the amine compound is represented by Formula 4: 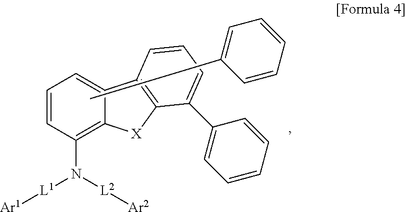
wherein in Formula 4, X, L¹, L², Ar¹, and Ar² are the same as respectively defined in Formula 1.

9. The light emitting element of claim 1, wherein L¹ and L² are each independently a direct linkage, a substituted or unsubstituted phenylene group, a substituted or unsubstituted biphenylene group, a substituted or unsubstituted terphenylene group, or a substituted or unsubstituted naphthylene group.

10. The light emitting element of claim 1, wherein R² and R³ are each independently a hydrogen atom, a deuterium atom, or a substituted or unsubstituted phenyl group, or at least one selected from among R² and R³ is combined with a phenyl group substituent to form a substituted or unsubstituted naphthyl group.

11. The light emitting element of claim 1, wherein Ar¹ and Ar² are each independently a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted fluorenyl group, a substituted or unsubstituted dibenzofuran group, a substituted or unsubstituted dibenzothiophene group, a substituted or unsubstituted benzonaphthofuran group, or a substituted or unsubstituted benzonaphthothiophene group.

12. The light emitting element of claim 2, wherein the emission layer comprises a compound represented by Formula E-1: 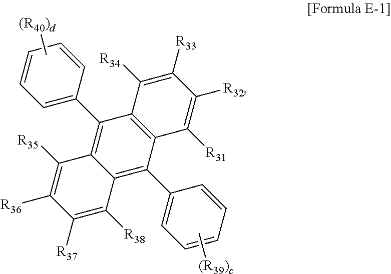
wherein in Formula E-1, R₃₁ to R₄₀ are each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, and/or combined with an adjacent group to form a ring, and
“c” and “d” are each independently an integer of 0 to 5.

13. The light emitting element of claim 1, wherein the amine compound is represented by any one selected from among compounds in Compound Group 1:

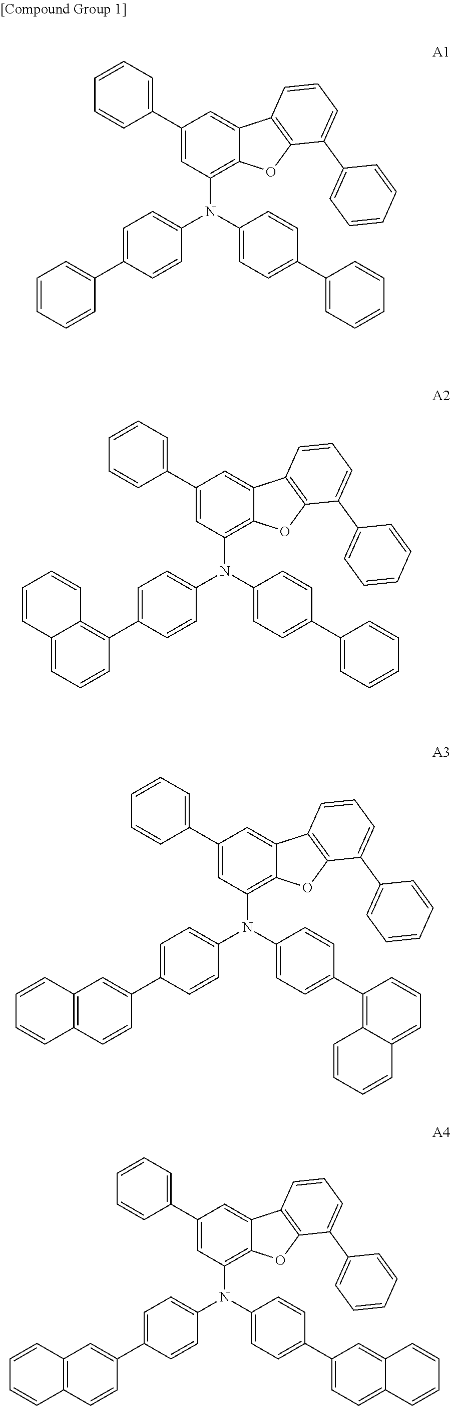 [SMILES: c1ccc(-c2ccc(N(c3ccc(-c4ccccc4)cc3)c3cc(-c4ccccc4)cc4c3oc3c(-c5ccccc5)cccc34)cc2)cc1 ; c1ccc(-c2ccc(N(c3ccc(-c4cccc5ccccc45)cc3)c3cc(-c4ccccc4)cc4c3oc3c(-c5ccccc5)cccc34)cc2)cc1 ; c1ccc(-c2cc(N(c3ccc(-c4ccc5ccccc5c4)cc3)c3ccc(-c4cccc5ccccc45)cc3)c3oc4c(-c5ccccc5)cccc4c3c2)cc1 ; c1ccc(-c2cc(N(c3ccc(-c4ccc5ccccc5c4)cc3)c3ccc(-c4ccc5ccccc5c4)cc3)c3oc4c(-c5ccccc5)cccc4c3c2)cc1]

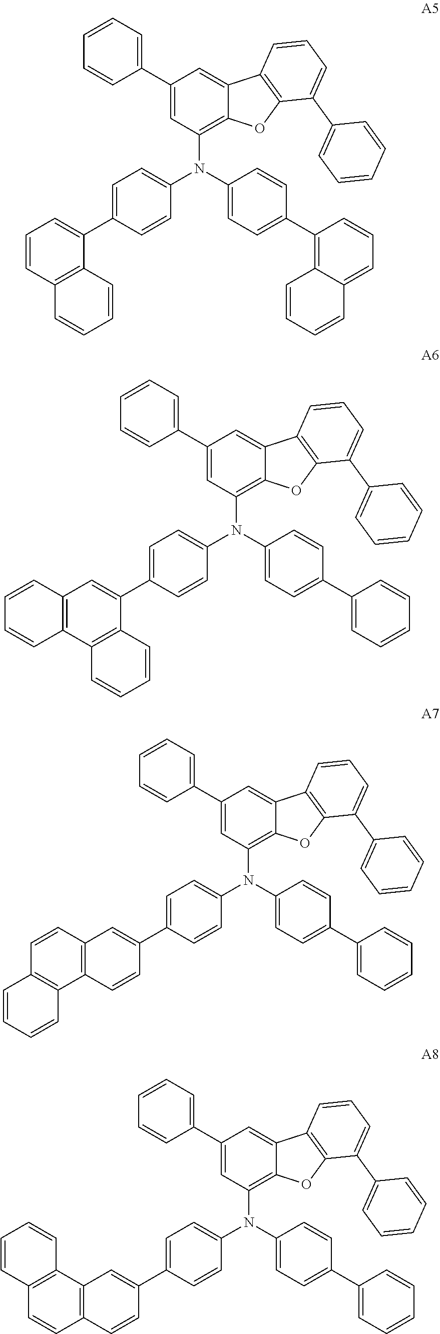 [SMILES: c1ccc(-c2cc(N(c3ccc(-c4cccc5ccccc45)cc3)c3ccc(-c4cccc5ccccc45)cc3)c3oc4c(-c5ccccc5)cccc4c3c2)cc1 ; c1ccc(-c2ccc(N(c3ccc(-c4cc5ccccc5c5ccccc45)cc3)c3cc(-c4ccccc4)cc4c3oc3c(-c5ccccc5)cccc34)cc2)cc1 ; c1ccc(-c2ccc(N(c3ccc(-c4ccc5c(ccc6ccccc65)c4)cc3)c3cc(-c4ccccc4)cc4c3oc3c(-c5ccccc5)cccc34)cc2)cc1 ; c1ccc(-c2ccc(N(c3ccc(-c4ccc5ccc6ccccc6c5c4)cc3)c3cc(-c4ccccc4)cc4c3oc3c(-c5ccccc5)cccc34)cc2)cc1]

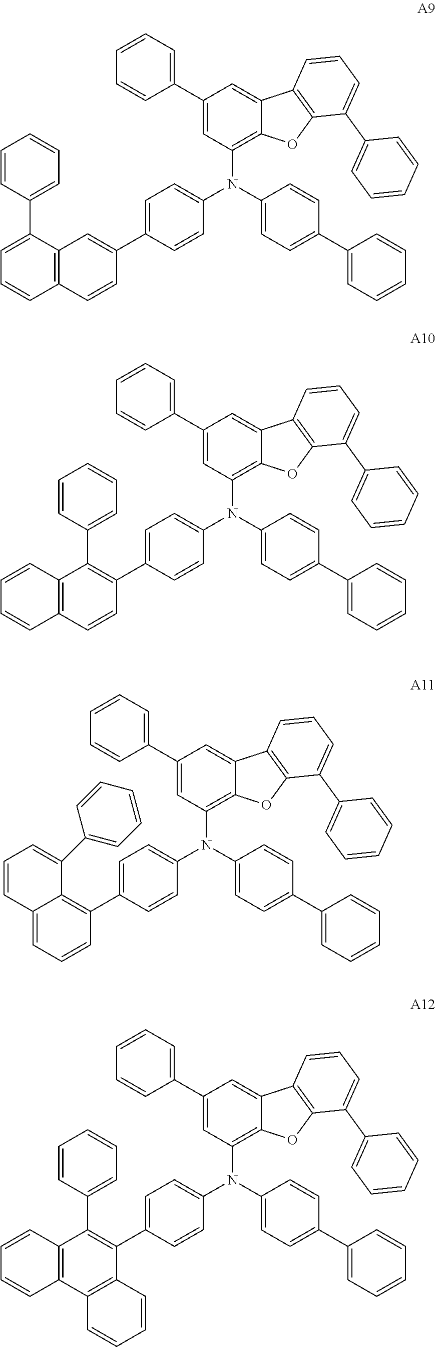 [SMILES: c1ccc(-c2ccc(N(c3ccc(-c4ccc5cccc(-c6ccccc6)c5c4)cc3)c3cc(-c4ccccc4)cc4c3oc3c(-c5ccccc5)cccc34)cc2)cc1 ; c1ccc(-c2ccc(N(c3ccc(-c4ccc5ccccc5c4-c4ccccc4)cc3)c3cc(-c4ccccc4)cc4c3oc3c(-c5ccccc5)cccc34)cc2)cc1 ; c1ccc(-c2ccc(N(c3ccc(-c4cccc5cccc(-c6ccccc6)c45)cc3)c3cc(-c4ccccc4)cc4c3oc3c(-c5ccccc5)cccc34)cc2)cc1 ; c1ccc(-c2ccc(N(c3ccc(-c4c(-c5ccccc5)c5ccccc5c5ccccc45)cc3)c3cc(-c4ccccc4)cc4c3oc3c(-c5ccccc5)cccc34)cc2)cc1]

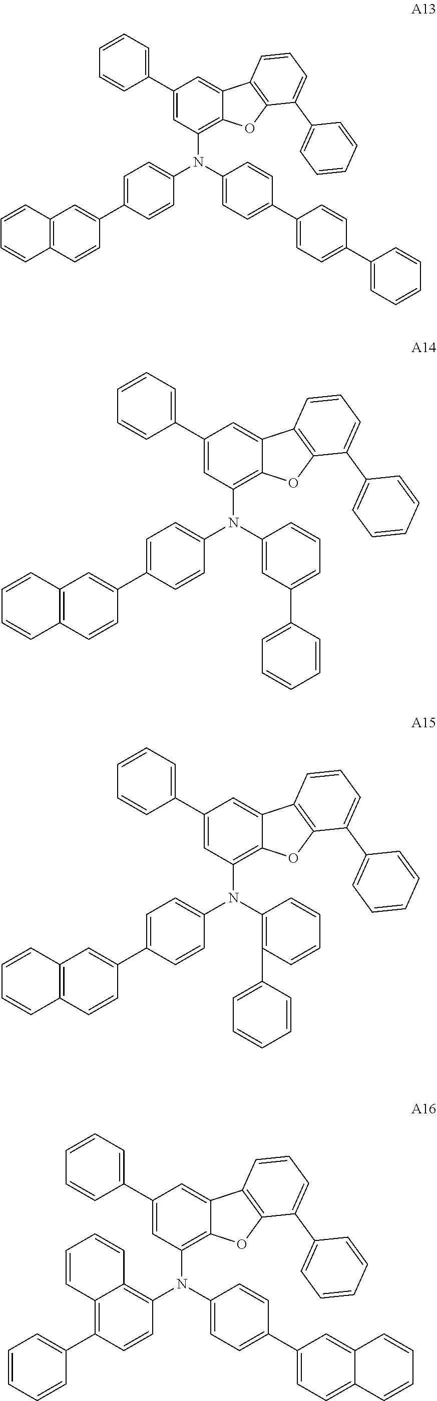 [SMILES: c1ccc(-c2ccc(-c3ccc(N(c4ccc(-c5ccc6ccccc6c5)cc4)c4cc(-c5ccccc5)cc5c4oc4c(-c6ccccc6)cccc45)cc3)cc2)cc1 ; c1ccc(-c2cccc(N(c3ccc(-c4ccc5ccccc5c4)cc3)c3cc(-c4ccccc4)cc4c3oc3c(-c5ccccc5)cccc34)c2)cc1 ; c1ccc(-c2cc(N(c3ccc(-c4ccc5ccccc5c4)cc3)c3ccccc3-c3ccccc3)c3oc4c(-c5ccccc5)cccc4c3c2)cc1 ; c1ccc(-c2cc(N(c3ccc(-c4ccc5ccccc5c4)cc3)c3ccc(-c4ccccc4)c4ccccc34)c3oc4c(-c5ccccc5)cccc4c3c2)cc1]

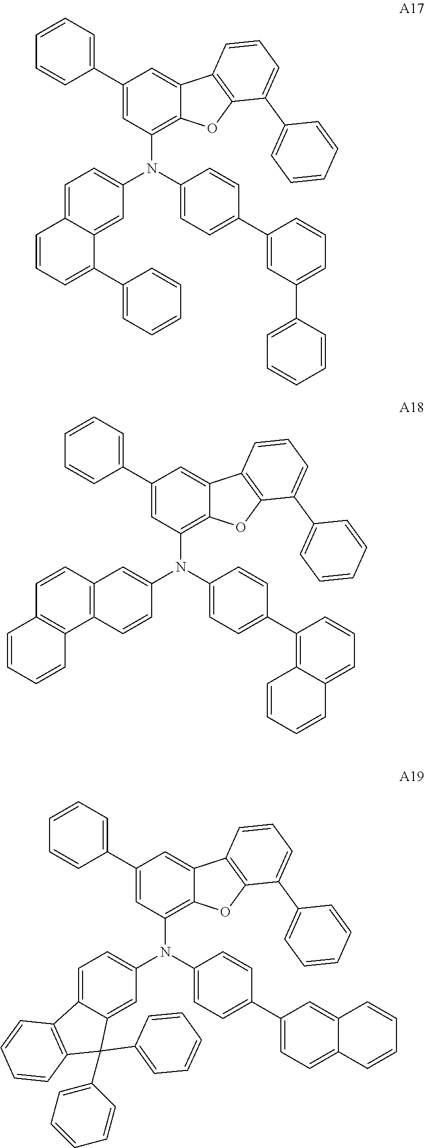 [SMILES: c1ccc(-c2cccc(-c3ccc(N(c4ccc5cccc(-c6ccccc6)c5c4)c4cc(-c5ccccc5)cc5c4oc4c(-c6ccccc6)cccc45)cc3)c2)cc1 ; c1ccc(-c2cc(N(c3ccc(-c4cccc5ccccc45)cc3)c3ccc4c(ccc5ccccc54)c3)c3oc4c(-c5ccccc5)cccc4c3c2)cc1 ; c1ccc(-c2cc(N(c3ccc(-c4ccc5ccccc5c4)cc3)c3ccc4c(c3)C(c3ccccc3)(c3ccccc3)c3ccccc3-4)c3oc4c(-c5ccccc5)cccc4c3c2)cc1]

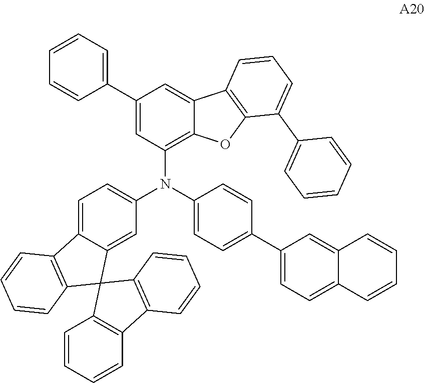 [SMILES: c1ccc(-c2cc(N(c3ccc(-c4ccc5ccccc5c4)cc3)c3ccc4c(c3)C3(c5ccccc5-c5ccccc53)c3ccccc3-4)c3oc4c(-c5ccccc5)cccc4c3c2)cc1]

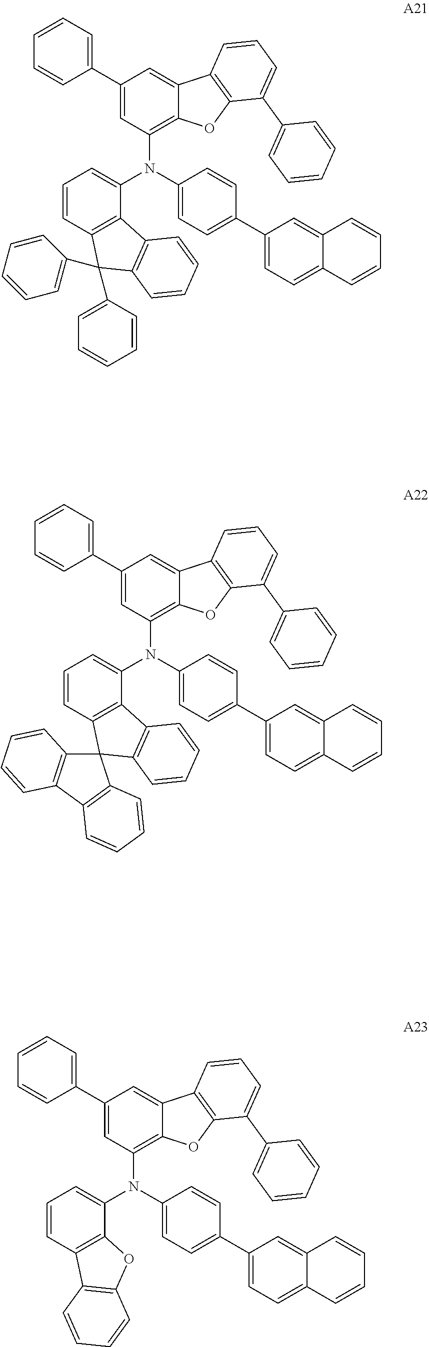 [SMILES: c1ccc(-c2cc(N(c3ccc(-c4ccc5ccccc5c4)cc3)c3cccc4c3-c3ccccc3C4(c3ccccc3)c3ccccc3)c3oc4c(-c5ccccc5)cccc4c3c2)cc1 ; c1ccc(-c2cc(N(c3ccc(-c4ccc5ccccc5c4)cc3)c3cccc4c3-c3ccccc3C43c4ccccc4-c4ccccc43)c3oc4c(-c5ccccc5)cccc4c3c2)cc1 ; c1ccc(-c2cc(N(c3ccc(-c4ccc5ccccc5c4)cc3)c3cccc4c3oc3ccccc34)c3oc4c(-c5ccccc5)cccc4c3c2)cc1]

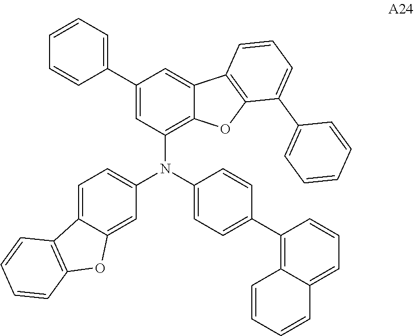 [SMILES: c1ccc(-c2cc(N(c3ccc(-c4cccc5ccccc45)cc3)c3ccc4c(c3)oc3ccccc34)c3oc4c(-c5ccccc5)cccc4c3c2)cc1]

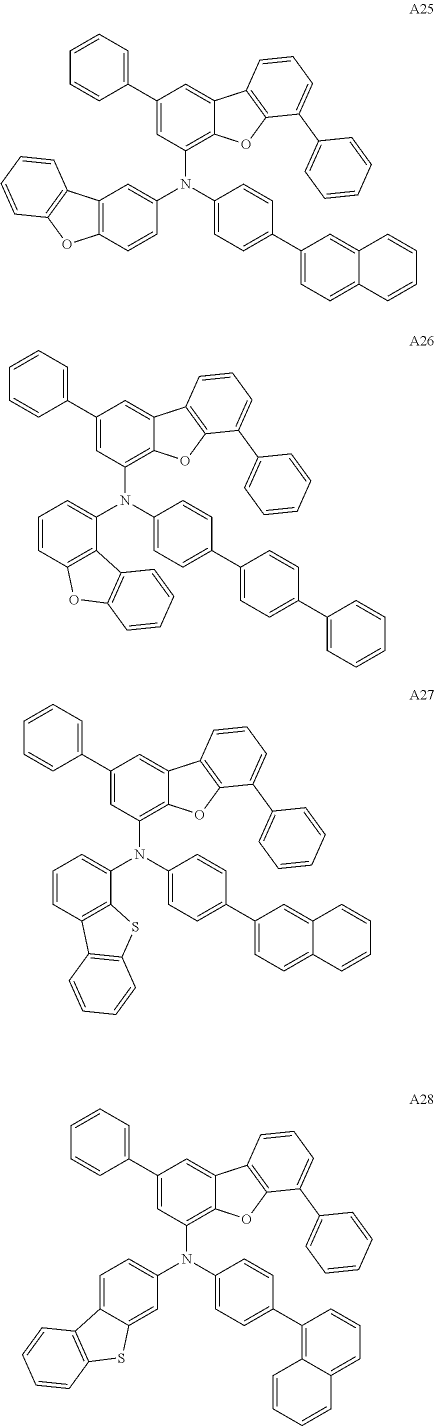 [SMILES: c1ccc(-c2cc(N(c3ccc(-c4ccc5ccccc5c4)cc3)c3ccc4oc5ccccc5c4c3)c3oc4c(-c5ccccc5)cccc4c3c2)cc1 ; c1ccc(-c2ccc(-c3ccc(N(c4cc(-c5ccccc5)cc5c4oc4c(-c6ccccc6)cccc45)c4cccc5oc6ccccc6c45)cc3)cc2)cc1 ; c1ccc(-c2cc(N(c3ccc(-c4ccc5ccccc5c4)cc3)c3cccc4c3sc3ccccc34)c3oc4c(-c5ccccc5)cccc4c3c2)cc1 ; c1ccc(-c2cc(N(c3ccc(-c4cccc5ccccc45)cc3)c3ccc4c(c3)sc3ccccc34)c3oc4c(-c5ccccc5)cccc4c3c2)cc1]

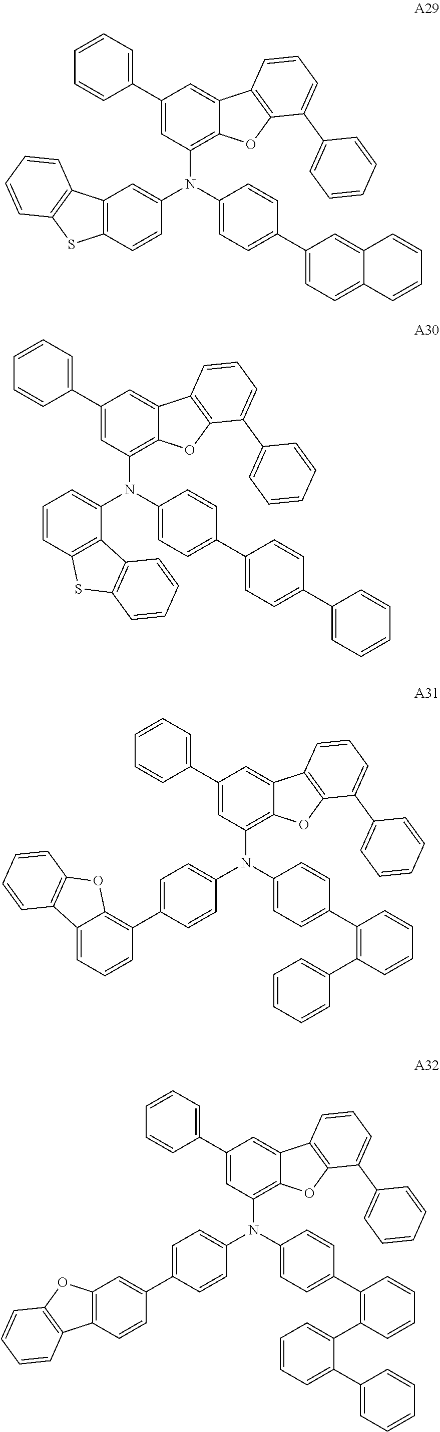 [SMILES: c1ccc(-c2cc(N(c3ccc(-c4ccc5ccccc5c4)cc3)c3ccc4sc5ccccc5c4c3)c3oc4c(-c5ccccc5)cccc4c3c2)cc1 ; c1ccc(-c2ccc(-c3ccc(N(c4cc(-c5ccccc5)cc5c4oc4c(-c6ccccc6)cccc45)c4cccc5sc6ccccc6c45)cc3)cc2)cc1 ; c1ccc(-c2cc(N(c3ccc(-c4ccccc4-c4ccccc4)cc3)c3ccc(-c4cccc5c4oc4ccccc45)cc3)c3oc4c(-c5ccccc5)cccc4c3c2)cc1 ; c1ccc(-c2cc(N(c3ccc(-c4ccc5c(c4)oc4ccccc45)cc3)c3ccc(-c4ccccc4-c4ccccc4-c4ccccc4)cc3)c3oc4c(-c5ccccc5)cccc4c3c2)cc1]

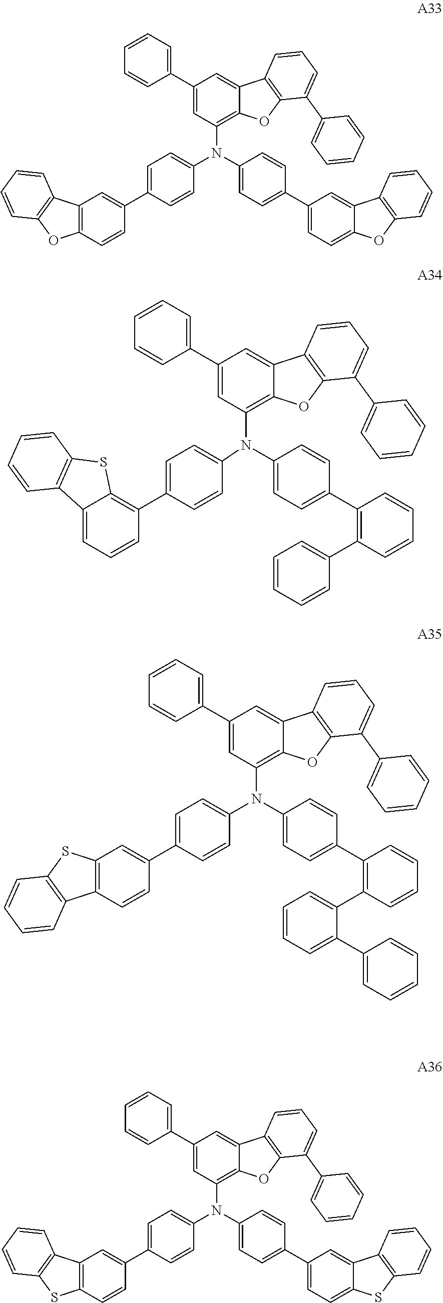 [SMILES: c1ccc(-c2cc(N(c3ccc(-c4ccc5oc6ccccc6c5c4)cc3)c3ccc(-c4ccc5oc6ccccc6c5c4)cc3)c3oc4c(-c5ccccc5)cccc4c3c2)cc1 ; c1ccc(-c2cc(N(c3ccc(-c4ccccc4-c4ccccc4)cc3)c3ccc(-c4cccc5c4sc4ccccc45)cc3)c3oc4c(-c5ccccc5)cccc4c3c2)cc1 ; c1ccc(-c2cc(N(c3ccc(-c4ccc5c(c4)sc4ccccc45)cc3)c3ccc(-c4ccccc4-c4ccccc4-c4ccccc4)cc3)c3oc4c(-c5ccccc5)cccc4c3c2)cc1 ; c1ccc(-c2cc(N(c3ccc(-c4ccc5sc6ccccc6c5c4)cc3)c3ccc(-c4ccc5sc6ccccc6c5c4)cc3)c3oc4c(-c5ccccc5)cccc4c3c2)cc1]

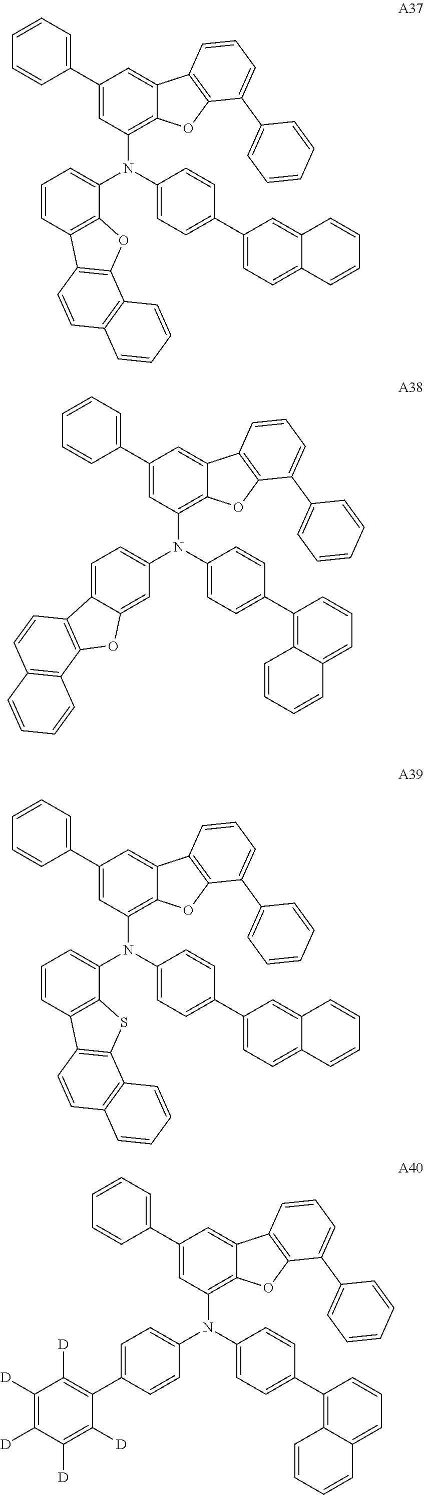

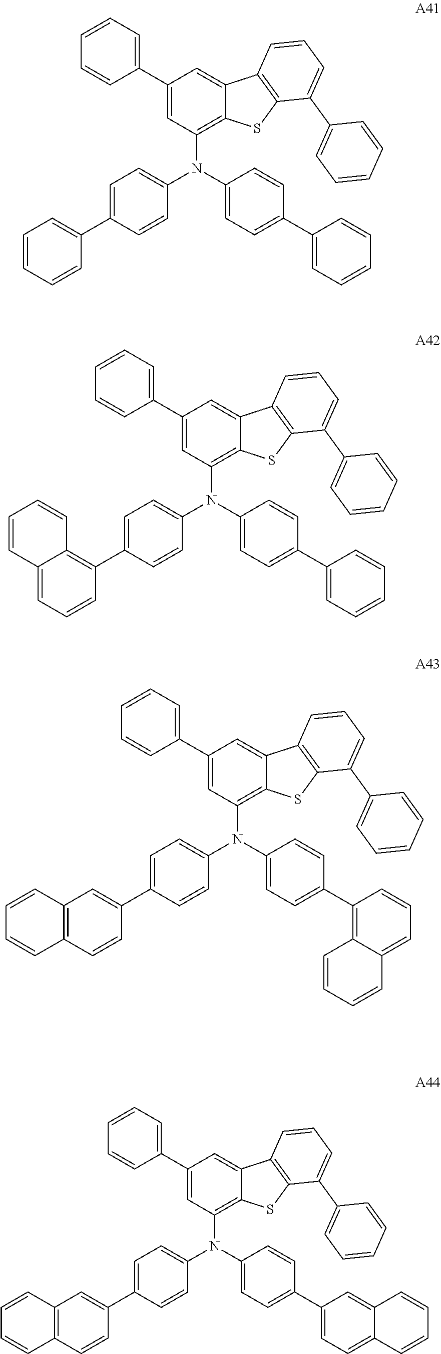 [SMILES: c1ccc(-c2ccc(N(c3ccc(-c4ccccc4)cc3)c3cc(-c4ccccc4)cc4c3sc3c(-c5ccccc5)cccc34)cc2)cc1 ; c1ccc(-c2ccc(N(c3ccc(-c4cccc5ccccc45)cc3)c3cc(-c4ccccc4)cc4c3sc3c(-c5ccccc5)cccc34)cc2)cc1 ; c1ccc(-c2cc(N(c3ccc(-c4ccc5ccccc5c4)cc3)c3ccc(-c4cccc5ccccc45)cc3)c3sc4c(-c5ccccc5)cccc4c3c2)cc1 ; c1ccc(-c2cc(N(c3ccc(-c4ccc5ccccc5c4)cc3)c3ccc(-c4ccc5ccccc5c4)cc3)c3sc4c(-c5ccccc5)cccc4c3c2)cc1]

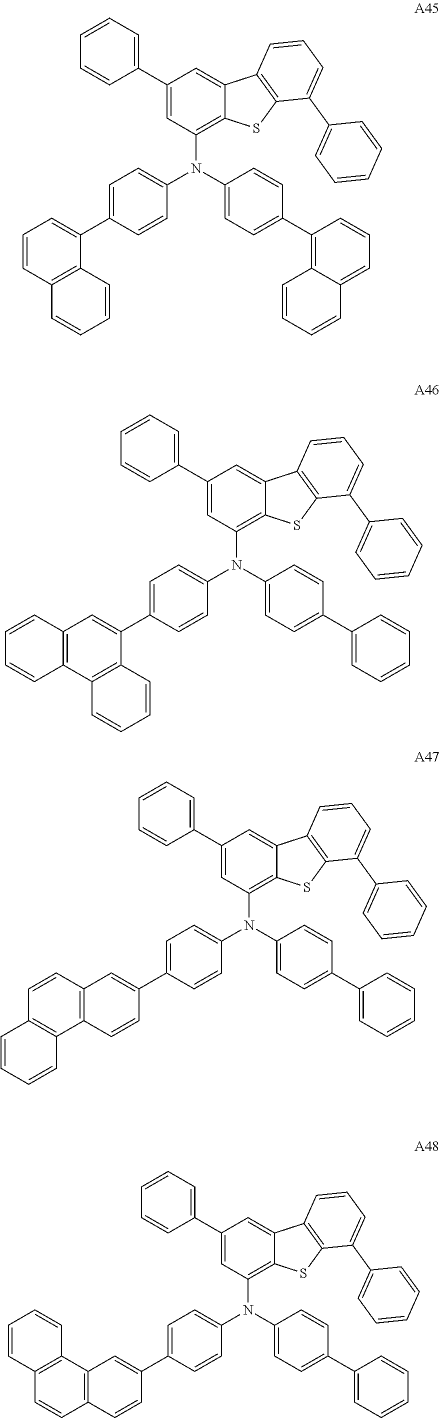 [SMILES: c1ccc(-c2cc(N(c3ccc(-c4cccc5ccccc45)cc3)c3ccc(-c4cccc5ccccc45)cc3)c3sc4c(-c5ccccc5)cccc4c3c2)cc1 ; c1ccc(-c2ccc(N(c3ccc(-c4cc5ccccc5c5ccccc45)cc3)c3cc(-c4ccccc4)cc4c3sc3c(-c5ccccc5)cccc34)cc2)cc1 ; c1ccc(-c2ccc(N(c3ccc(-c4ccc5c(ccc6ccccc65)c4)cc3)c3cc(-c4ccccc4)cc4c3sc3c(-c5ccccc5)cccc34)cc2)cc1 ; c1ccc(-c2ccc(N(c3ccc(-c4ccc5ccc6ccccc6c5c4)cc3)c3cc(-c4ccccc4)cc4c3sc3c(-c5ccccc5)cccc34)cc2)cc1]

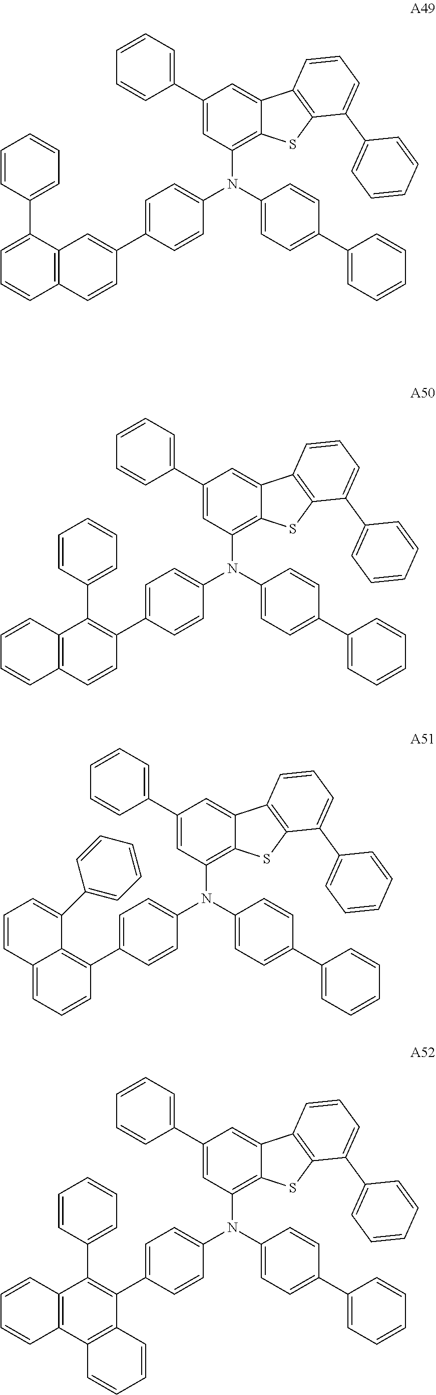 [SMILES: c1ccc(-c2ccc(N(c3ccc(-c4ccc5cccc(-c6ccccc6)c5c4)cc3)c3cc(-c4ccccc4)cc4c3sc3c(-c5ccccc5)cccc34)cc2)cc1 ; c1ccc(-c2ccc(N(c3ccc(-c4ccc5ccccc5c4-c4ccccc4)cc3)c3cc(-c4ccccc4)cc4c3sc3c(-c5ccccc5)cccc34)cc2)cc1 ; c1ccc(-c2ccc(N(c3ccc(-c4cccc5cccc(-c6ccccc6)c45)cc3)c3cc(-c4ccccc4)cc4c3sc3c(-c5ccccc5)cccc34)cc2)cc1 ; c1ccc(-c2ccc(N(c3ccc(-c4c(-c5ccccc5)c5ccccc5c5ccccc45)cc3)c3cc(-c4ccccc4)cc4c3sc3c(-c5ccccc5)cccc34)cc2)cc1]

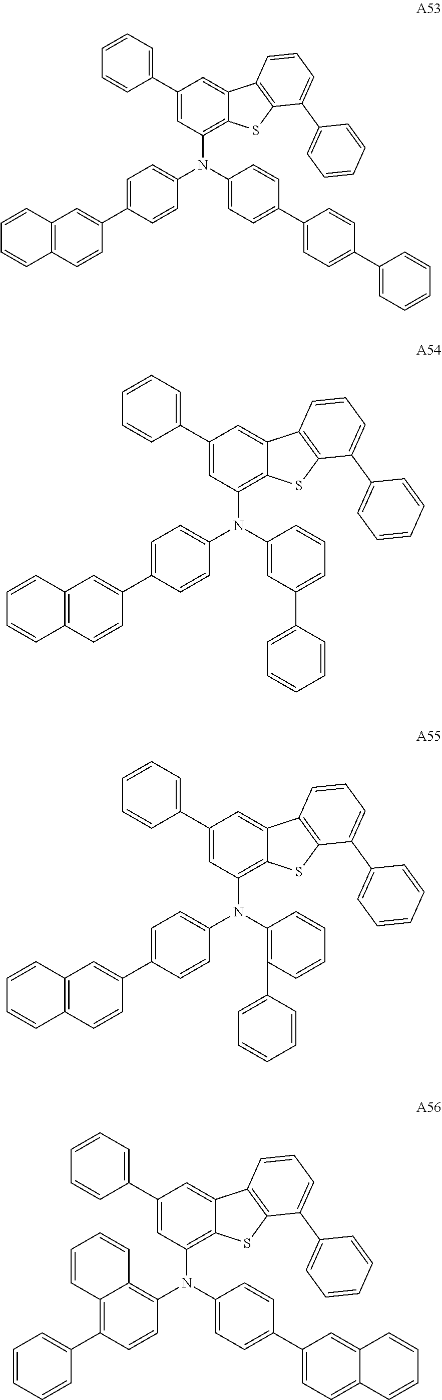 [SMILES: c1ccc(-c2ccc(-c3ccc(N(c4ccc(-c5ccc6ccccc6c5)cc4)c4cc(-c5ccccc5)cc5c4sc4c(-c6ccccc6)cccc45)cc3)cc2)cc1 ; c1ccc(-c2cccc(N(c3ccc(-c4ccc5ccccc5c4)cc3)c3cc(-c4ccccc4)cc4c3sc3c(-c5ccccc5)cccc34)c2)cc1 ; c1ccc(-c2cc(N(c3ccc(-c4ccc5ccccc5c4)cc3)c3ccccc3-c3ccccc3)c3sc4c(-c5ccccc5)cccc4c3c2)cc1 ; c1ccc(-c2cc(N(c3ccc(-c4ccc5ccccc5c4)cc3)c3ccc(-c4ccccc4)c4ccccc34)c3sc4c(-c5ccccc5)cccc4c3c2)cc1]

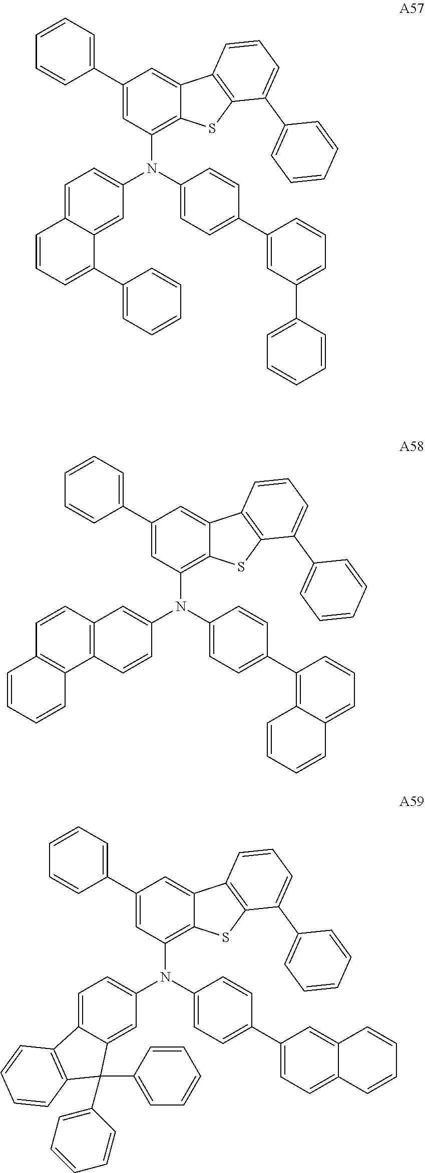 [SMILES: c1ccc(-c2cccc(-c3ccc(N(c4ccc5cccc(-c6ccccc6)c5c4)c4cc(-c5ccccc5)cc5c4sc4c(-c6ccccc6)cccc45)cc3)c2)cc1 ; c1ccc(-c2cc(N(c3ccc(-c4cccc5ccccc45)cc3)c3ccc4c(ccc5ccccc54)c3)c3sc4c(-c5ccccc5)cccc4c3c2)cc1 ; c1ccc(-c2cc(N(c3ccc(-c4ccc5ccccc5c4)cc3)c3ccc4c(c3)C(c3ccccc3)(c3ccccc3)c3ccccc3-4)c3sc4c(-c5ccccc5)cccc4c3c2)cc1]

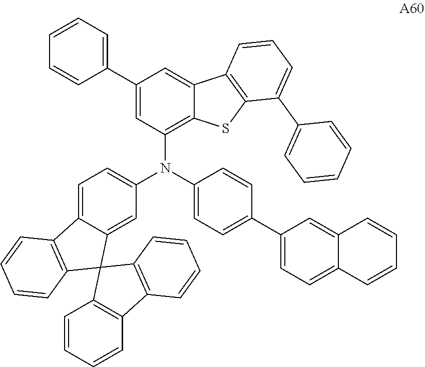 [SMILES: c1ccc(-c2cc(N(c3ccc(-c4ccc5ccccc5c4)cc3)c3ccc4c(c3)C3(c5ccccc5-c5ccccc53)c3ccccc3-4)c3sc4c(-c5ccccc5)cccc4c3c2)cc1]

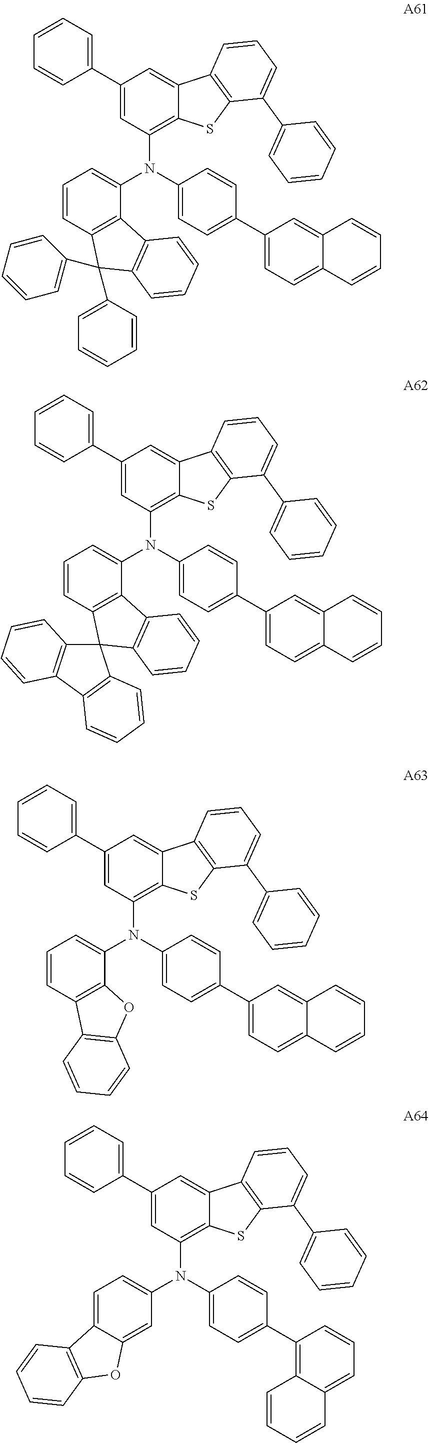

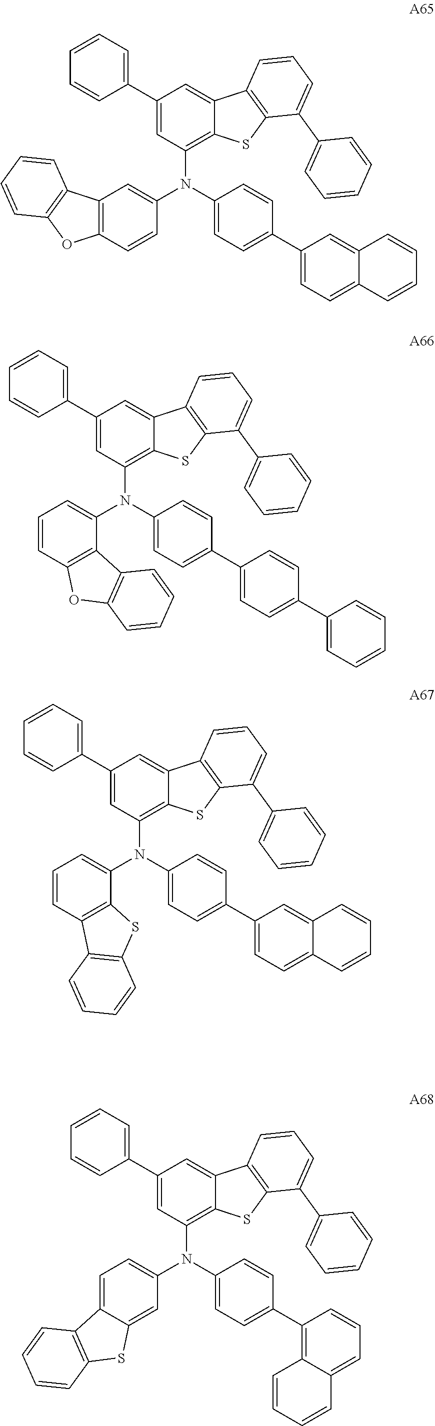 [SMILES: c1ccc(-c2cc(N(c3ccc(-c4ccc5ccccc5c4)cc3)c3ccc4oc5ccccc5c4c3)c3sc4c(-c5ccccc5)cccc4c3c2)cc1 ; c1ccc(-c2ccc(-c3ccc(N(c4cc(-c5ccccc5)cc5c4sc4c(-c6ccccc6)cccc45)c4cccc5oc6ccccc6c45)cc3)cc2)cc1 ; c1ccc(-c2cc(N(c3ccc(-c4ccc5ccccc5c4)cc3)c3cccc4c3sc3ccccc34)c3sc4c(-c5ccccc5)cccc4c3c2)cc1 ; c1ccc(-c2cc(N(c3ccc(-c4cccc5ccccc45)cc3)c3ccc4c(c3)sc3ccccc34)c3sc4c(-c5ccccc5)cccc4c3c2)cc1]

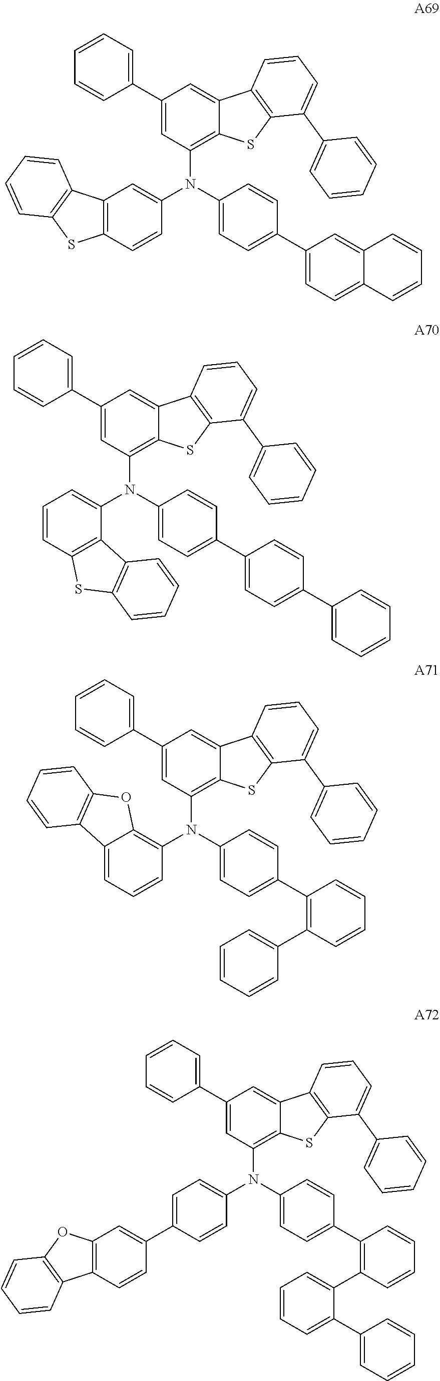 [SMILES: c1ccc(-c2cc(N(c3ccc(-c4ccc5ccccc5c4)cc3)c3ccc4sc5ccccc5c4c3)c3sc4c(-c5ccccc5)cccc4c3c2)cc1 ; c1ccc(-c2ccc(-c3ccc(N(c4cc(-c5ccccc5)cc5c4sc4c(-c6ccccc6)cccc45)c4cccc5sc6ccccc6c45)cc3)cc2)cc1 ; c1ccc(-c2cc(N(c3ccc(-c4ccccc4-c4ccccc4)cc3)c3cccc4c3oc3ccccc34)c3sc4c(-c5ccccc5)cccc4c3c2)cc1 ; c1ccc(-c2cc(N(c3ccc(-c4ccc5c(c4)oc4ccccc45)cc3)c3ccc(-c4ccccc4-c4ccccc4-c4ccccc4)cc3)c3sc4c(-c5ccccc5)cccc4c3c2)cc1]

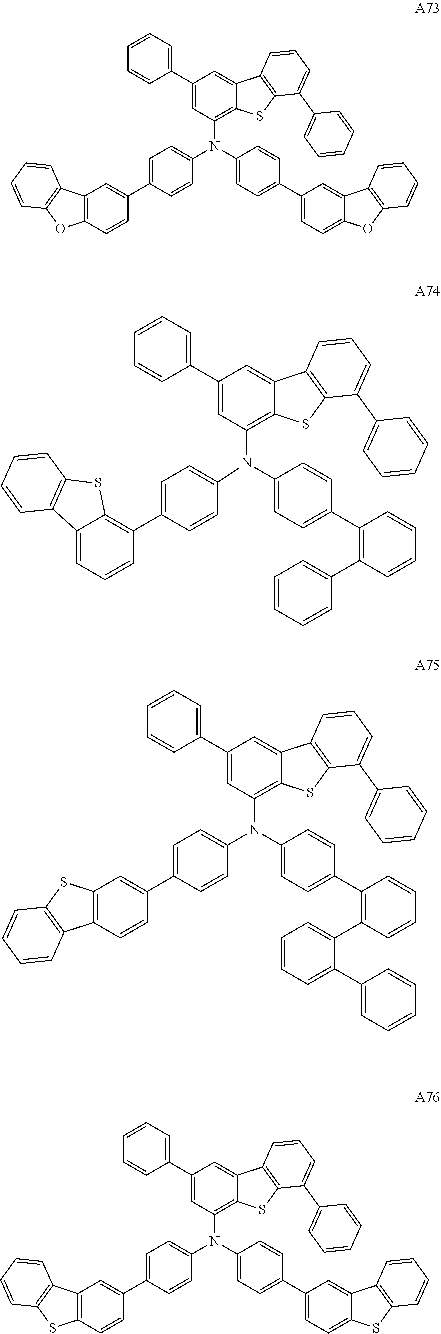 [SMILES: c1ccc(-c2cc(N(c3ccc(-c4ccc5oc6ccccc6c5c4)cc3)c3ccc(-c4ccc5oc6ccccc6c5c4)cc3)c3sc4c(-c5ccccc5)cccc4c3c2)cc1 ; c1ccc(-c2cc(N(c3ccc(-c4ccccc4-c4ccccc4)cc3)c3ccc(-c4cccc5c4sc4ccccc45)cc3)c3sc4c(-c5ccccc5)cccc4c3c2)cc1 ; c1ccc(-c2cc(N(c3ccc(-c4ccc5c(c4)sc4ccccc45)cc3)c3ccc(-c4ccccc4-c4ccccc4-c4ccccc4)cc3)c3sc4c(-c5ccccc5)cccc4c3c2)cc1 ; c1ccc(-c2cc(N(c3ccc(-c4ccc5sc6ccccc6c5c4)cc3)c3ccc(-c4ccc5sc6ccccc6c5c4)cc3)c3sc4c(-c5ccccc5)cccc4c3c2)cc1]

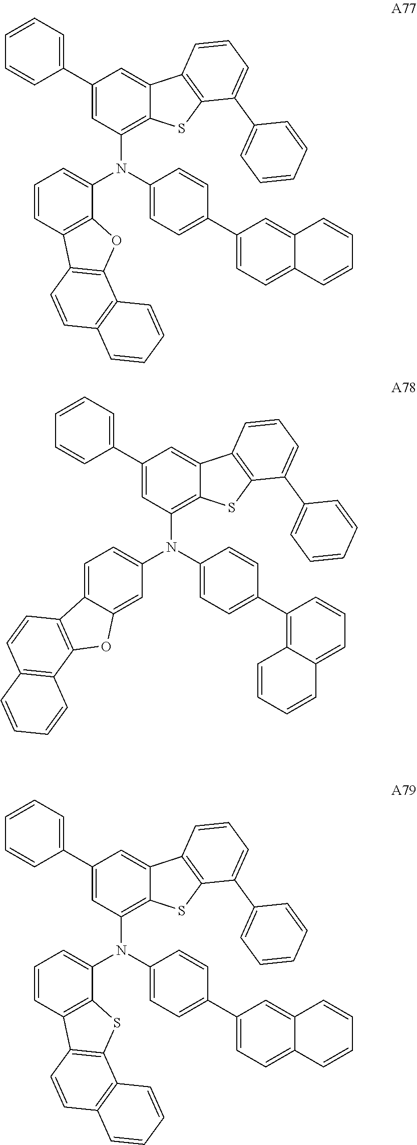

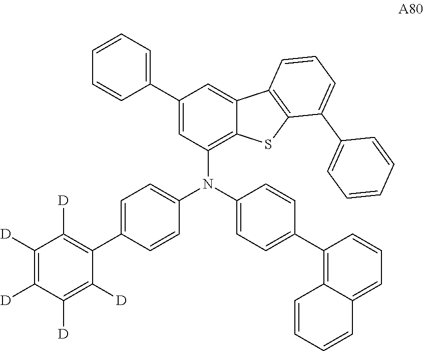 [SMILES: [2H]c1c([2H])c([2H])c(-c2ccc(N(c3ccc(-c4cccc5ccccc45)cc3)c3cc(-c4ccccc4)cc4c3sc3c(-c5ccccc5)cccc34)cc2)c([2H])c1[2H]]

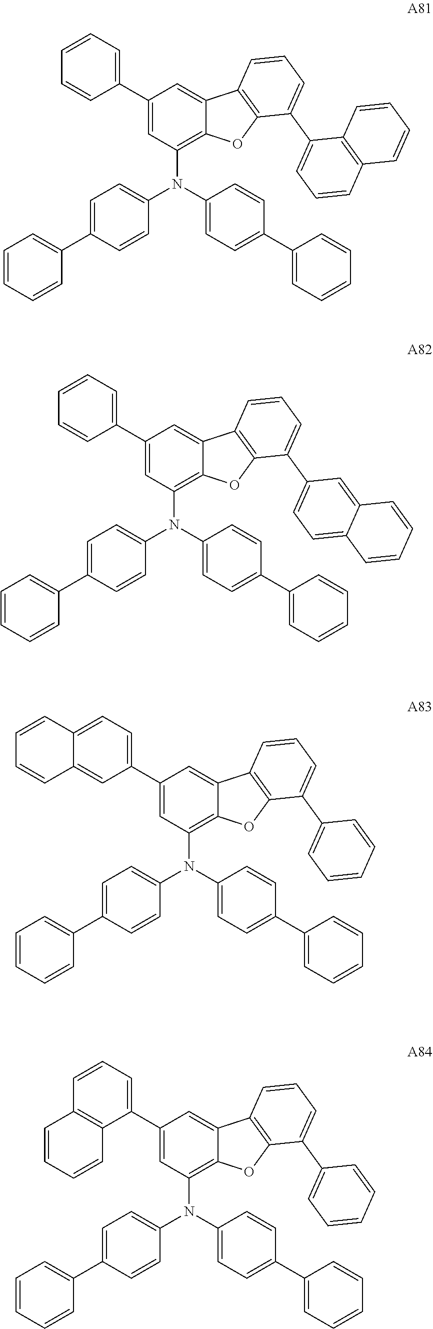 [SMILES: c1ccc(-c2ccc(N(c3ccc(-c4ccccc4)cc3)c3cc(-c4ccccc4)cc4c3oc3c(-c5cccc6ccccc56)cccc34)cc2)cc1 ; c1ccc(-c2ccc(N(c3ccc(-c4ccccc4)cc3)c3cc(-c4ccccc4)cc4c3oc3c(-c5ccc6ccccc6c5)cccc34)cc2)cc1 ; c1ccc(-c2ccc(N(c3ccc(-c4ccccc4)cc3)c3cc(-c4ccc5ccccc5c4)cc4c3oc3c(-c5ccccc5)cccc34)cc2)cc1 ; c1ccc(-c2ccc(N(c3ccc(-c4ccccc4)cc3)c3cc(-c4cccc5ccccc45)cc4c3oc3c(-c5ccccc5)cccc34)cc2)cc1]

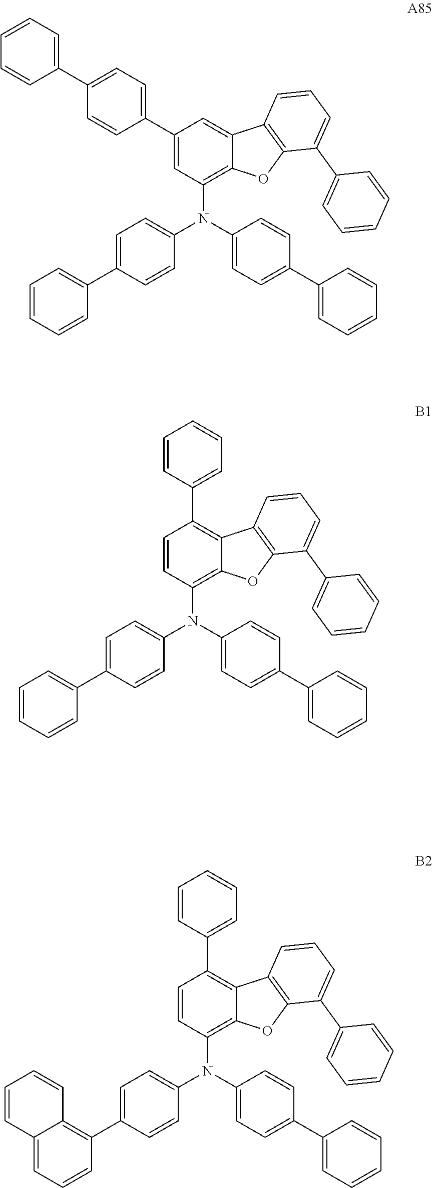 [SMILES: c1ccc(-c2ccc(-c3cc(N(c4ccc(-c5ccccc5)cc4)c4ccc(-c5ccccc5)cc4)c4oc5c(-c6ccccc6)cccc5c4c3)cc2)cc1 ; c1ccc(-c2ccc(N(c3ccc(-c4ccccc4)cc3)c3ccc(-c4ccccc4)c4c3oc3c(-c5ccccc5)cccc34)cc2)cc1 ; c1ccc(-c2ccc(N(c3ccc(-c4cccc5ccccc45)cc3)c3ccc(-c4ccccc4)c4c3oc3c(-c5ccccc5)cccc34)cc2)cc1]

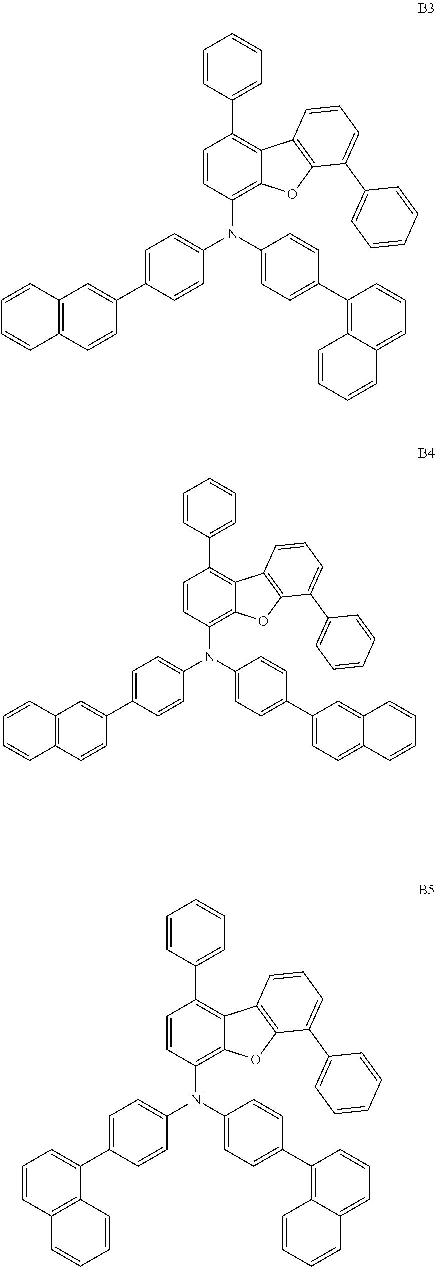 [SMILES: c1ccc(-c2cccc3c2oc2c(N(c4ccc(-c5ccc6ccccc6c5)cc4)c4ccc(-c5cccc6ccccc56)cc4)ccc(-c4ccccc4)c23)cc1 ; c1ccc(-c2cccc3c2oc2c(N(c4ccc(-c5ccc6ccccc6c5)cc4)c4ccc(-c5ccc6ccccc6c5)cc4)ccc(-c4ccccc4)c23)cc1 ; c1ccc(-c2cccc3c2oc2c(N(c4ccc(-c5cccc6ccccc56)cc4)c4ccc(-c5cccc6ccccc56)cc4)ccc(-c4ccccc4)c23)cc1]

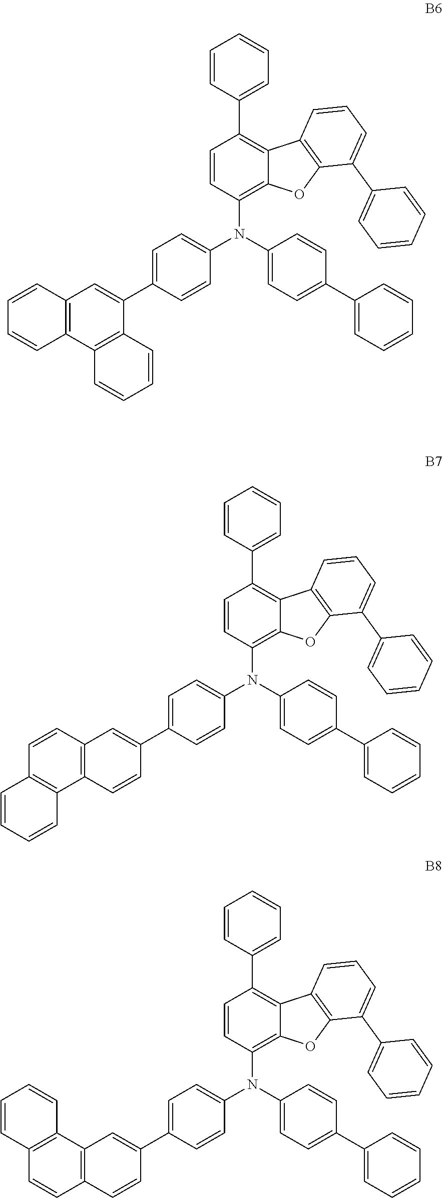 [SMILES: c1ccc(-c2ccc(N(c3ccc(-c4cc5ccccc5c5ccccc45)cc3)c3ccc(-c4ccccc4)c4c3oc3c(-c5ccccc5)cccc34)cc2)cc1 ; c1ccc(-c2ccc(N(c3ccc(-c4ccc5c(ccc6ccccc65)c4)cc3)c3ccc(-c4ccccc4)c4c3oc3c(-c5ccccc5)cccc34)cc2)cc1 ; c1ccc(-c2ccc(N(c3ccc(-c4ccc5ccc6ccccc6c5c4)cc3)c3ccc(-c4ccccc4)c4c3oc3c(-c5ccccc5)cccc34)cc2)cc1]

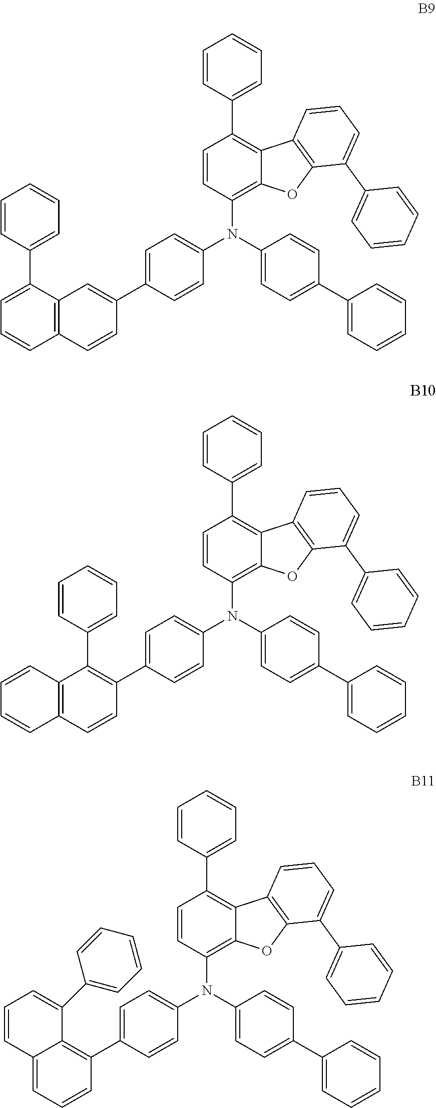 [SMILES: c1ccc(-c2ccc(N(c3ccc(-c4ccc5cccc(-c6ccccc6)c5c4)cc3)c3ccc(-c4ccccc4)c4c3oc3c(-c5ccccc5)cccc34)cc2)cc1 ; c1ccc(-c2ccc(N(c3ccc(-c4ccc5ccccc5c4-c4ccccc4)cc3)c3ccc(-c4ccccc4)c4c3oc3c(-c5ccccc5)cccc34)cc2)cc1 ; c1ccc(-c2ccc(N(c3ccc(-c4cccc5cccc(-c6ccccc6)c45)cc3)c3ccc(-c4ccccc4)c4c3oc3c(-c5ccccc5)cccc34)cc2)cc1]

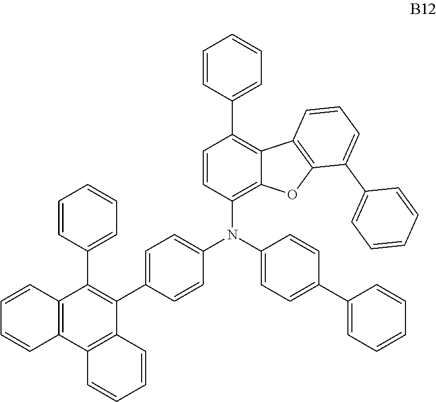 [SMILES: c1ccc(-c2ccc(N(c3ccc(-c4c(-c5ccccc5)c5ccccc5c5ccccc45)cc3)c3ccc(-c4ccccc4)c4c3oc3c(-c5ccccc5)cccc34)cc2)cc1]

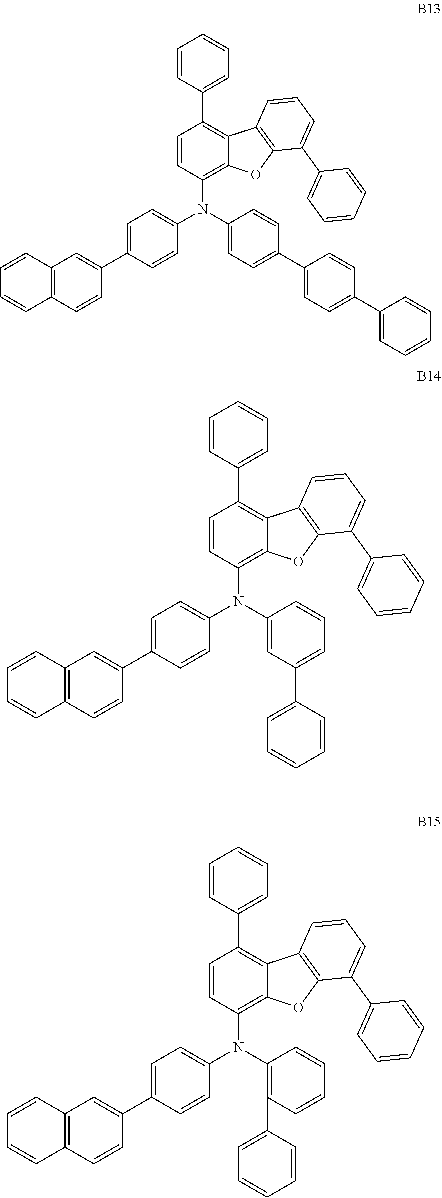 [SMILES: c1ccc(-c2ccc(-c3ccc(N(c4ccc(-c5ccc6ccccc6c5)cc4)c4ccc(-c5ccccc5)c5c4oc4c(-c6ccccc6)cccc45)cc3)cc2)cc1 ; c1ccc(-c2cccc(N(c3ccc(-c4ccc5ccccc5c4)cc3)c3ccc(-c4ccccc4)c4c3oc3c(-c5ccccc5)cccc34)c2)cc1 ; c1ccc(-c2ccccc2N(c2ccc(-c3ccc4ccccc4c3)cc2)c2ccc(-c3ccccc3)c3c2oc2c(-c4ccccc4)cccc23)cc1]

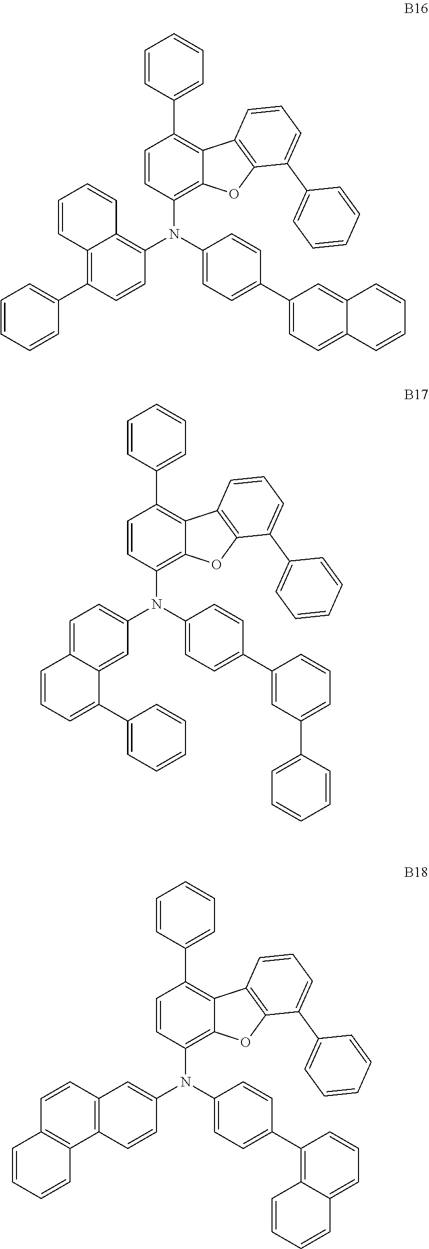 [SMILES: c1ccc(-c2ccc(N(c3ccc(-c4ccc5ccccc5c4)cc3)c3ccc(-c4ccccc4)c4c3oc3c(-c5ccccc5)cccc34)c3ccccc23)cc1 ; c1ccc(-c2cccc(-c3ccc(N(c4ccc5cccc(-c6ccccc6)c5c4)c4ccc(-c5ccccc5)c5c4oc4c(-c6ccccc6)cccc45)cc3)c2)cc1 ; c1ccc(-c2cccc3c2oc2c(N(c4ccc(-c5cccc6ccccc56)cc4)c4ccc5c(ccc6ccccc65)c4)ccc(-c4ccccc4)c23)cc1]

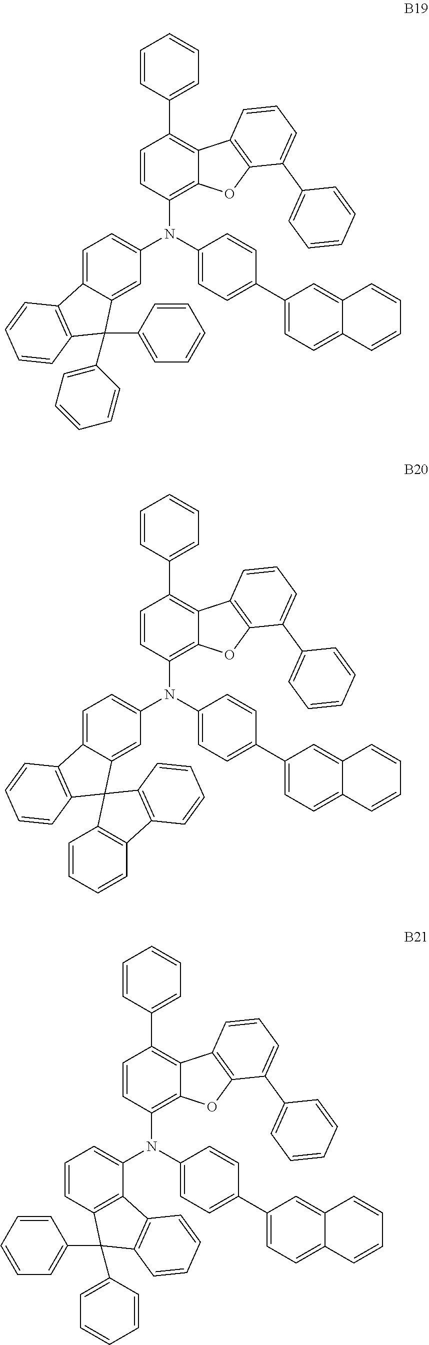 [SMILES: c1ccc(-c2cccc3c2oc2c(N(c4ccc(-c5ccc6ccccc6c5)cc4)c4ccc5c(c4)C(c4ccccc4)(c4ccccc4)c4ccccc4-5)ccc(-c4ccccc4)c23)cc1 ; c1ccc(-c2cccc3c2oc2c(N(c4ccc(-c5ccc6ccccc6c5)cc4)c4ccc5c(c4)C4(c6ccccc6-c6ccccc64)c4ccccc4-5)ccc(-c4ccccc4)c23)cc1 ; c1ccc(-c2cccc3c2oc2c(N(c4ccc(-c5ccc6ccccc6c5)cc4)c4cccc5c4-c4ccccc4C5(c4ccccc4)c4ccccc4)ccc(-c4ccccc4)c23)cc1]

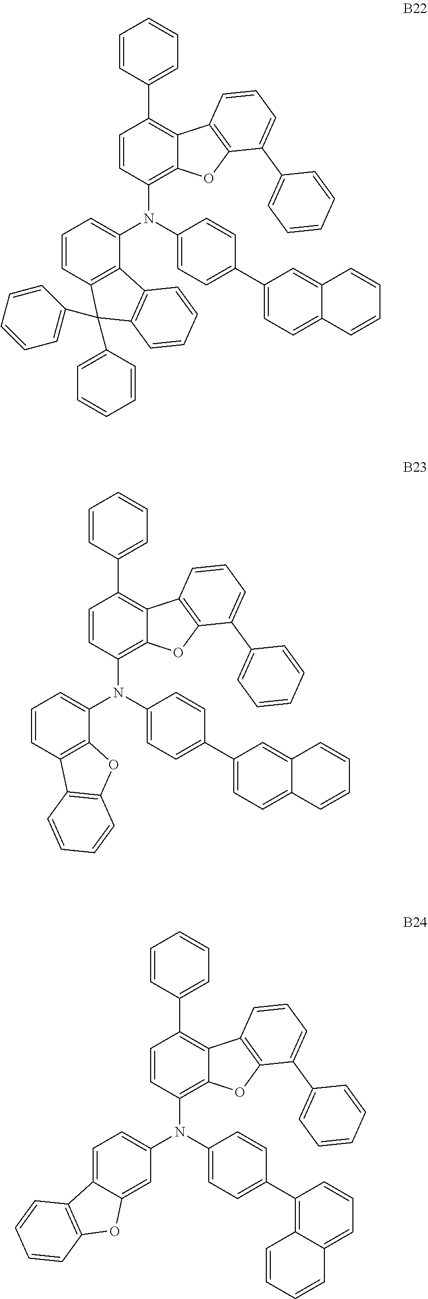 [SMILES: c1ccc(-c2cccc3c2oc2c(N(c4ccc(-c5ccc6ccccc6c5)cc4)c4cccc5c4-c4ccccc4C5(c4ccccc4)c4ccccc4)ccc(-c4ccccc4)c23)cc1 ; c1ccc(-c2cccc3c2oc2c(N(c4ccc(-c5ccc6ccccc6c5)cc4)c4cccc5c4oc4ccccc45)ccc(-c4ccccc4)c23)cc1 ; c1ccc(-c2cccc3c2oc2c(N(c4ccc(-c5cccc6ccccc56)cc4)c4ccc5c(c4)oc4ccccc45)ccc(-c4ccccc4)c23)cc1]

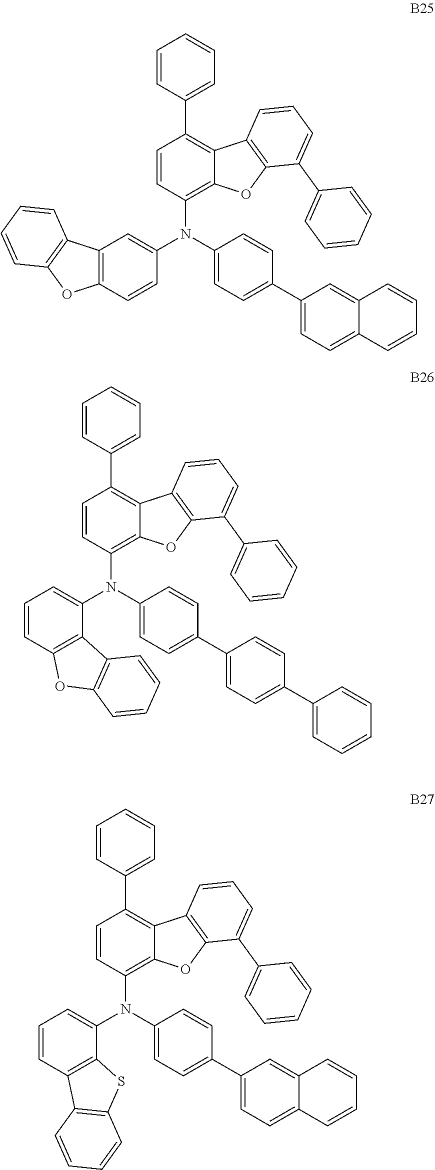 [SMILES: c1ccc(-c2cccc3c2oc2c(N(c4ccc(-c5ccc6ccccc6c5)cc4)c4ccc5oc6ccccc6c5c4)ccc(-c4ccccc4)c23)cc1 ; c1ccc(-c2ccc(-c3ccc(N(c4ccc(-c5ccccc5)c5c4oc4c(-c6ccccc6)cccc45)c4cccc5oc6ccccc6c45)cc3)cc2)cc1 ; c1ccc(-c2cccc3c2oc2c(N(c4ccc(-c5ccc6ccccc6c5)cc4)c4cccc5c4sc4ccccc45)ccc(-c4ccccc4)c23)cc1]

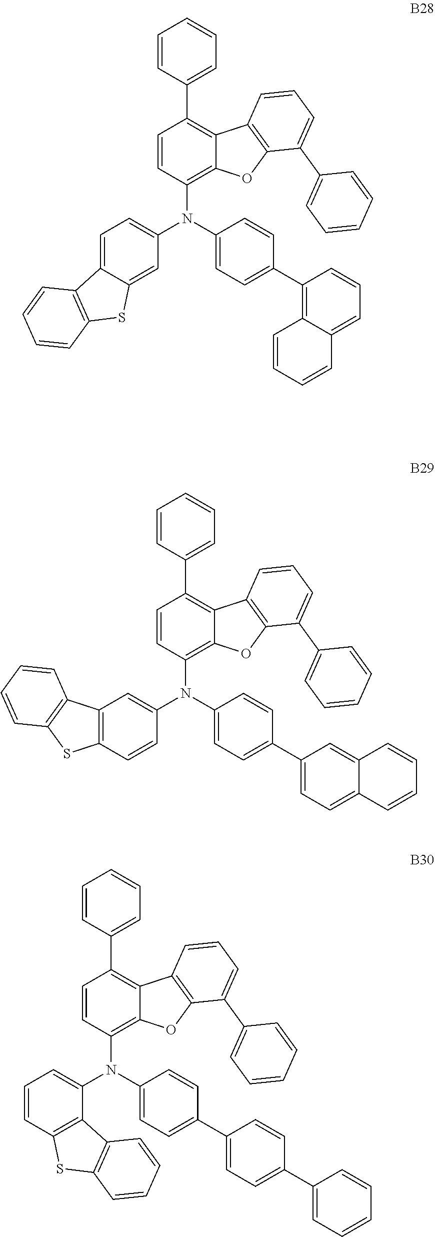 [SMILES: c1ccc(-c2cccc3c2oc2c(N(c4ccc(-c5cccc6ccccc56)cc4)c4ccc5c(c4)sc4ccccc45)ccc(-c4ccccc4)c23)cc1 ; c1ccc(-c2cccc3c2oc2c(N(c4ccc(-c5ccc6ccccc6c5)cc4)c4ccc5sc6ccccc6c5c4)ccc(-c4ccccc4)c23)cc1 ; c1ccc(-c2ccc(-c3ccc(N(c4ccc(-c5ccccc5)c5c4oc4c(-c6ccccc6)cccc45)c4cccc5sc6ccccc6c45)cc3)cc2)cc1]

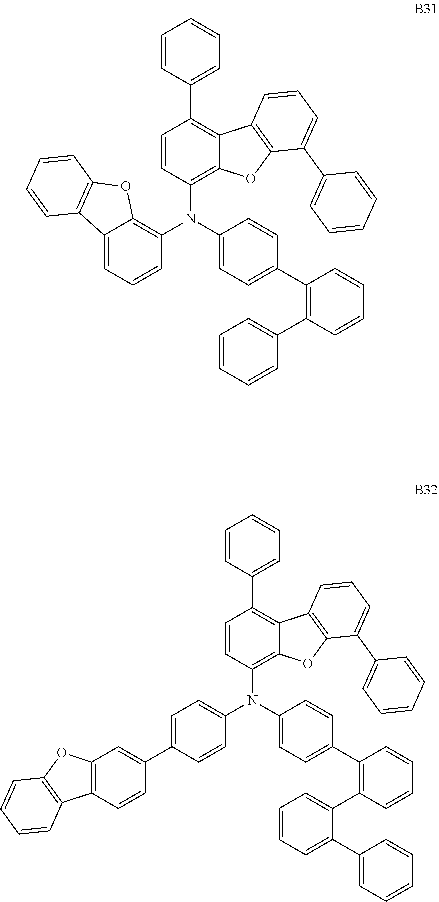 [SMILES: c1ccc(-c2ccccc2-c2ccc(N(c3cccc4c3oc3ccccc34)c3ccc(-c4ccccc4)c4c3oc3c(-c5ccccc5)cccc34)cc2)cc1 ; c1ccc(-c2ccccc2-c2ccccc2-c2ccc(N(c3ccc(-c4ccc5c(c4)oc4ccccc45)cc3)c3ccc(-c4ccccc4)c4c3oc3c(-c5ccccc5)cccc34)cc2)cc1]

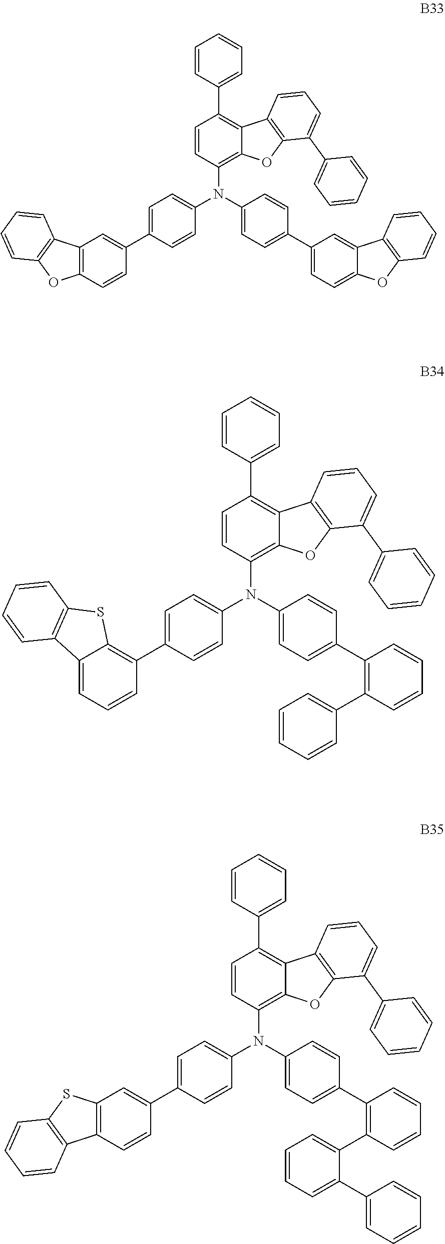 [SMILES: c1ccc(-c2cccc3c2oc2c(N(c4ccc(-c5ccc6oc7ccccc7c6c5)cc4)c4ccc(-c5ccc6oc7ccccc7c6c5)cc4)ccc(-c4ccccc4)c23)cc1 ; c1ccc(-c2ccccc2-c2ccc(N(c3ccc(-c4cccc5c4sc4ccccc45)cc3)c3ccc(-c4ccccc4)c4c3oc3c(-c5ccccc5)cccc34)cc2)cc1 ; c1ccc(-c2ccccc2-c2ccccc2-c2ccc(N(c3ccc(-c4ccc5c(c4)sc4ccccc45)cc3)c3ccc(-c4ccccc4)c4c3oc3c(-c5ccccc5)cccc34)cc2)cc1]

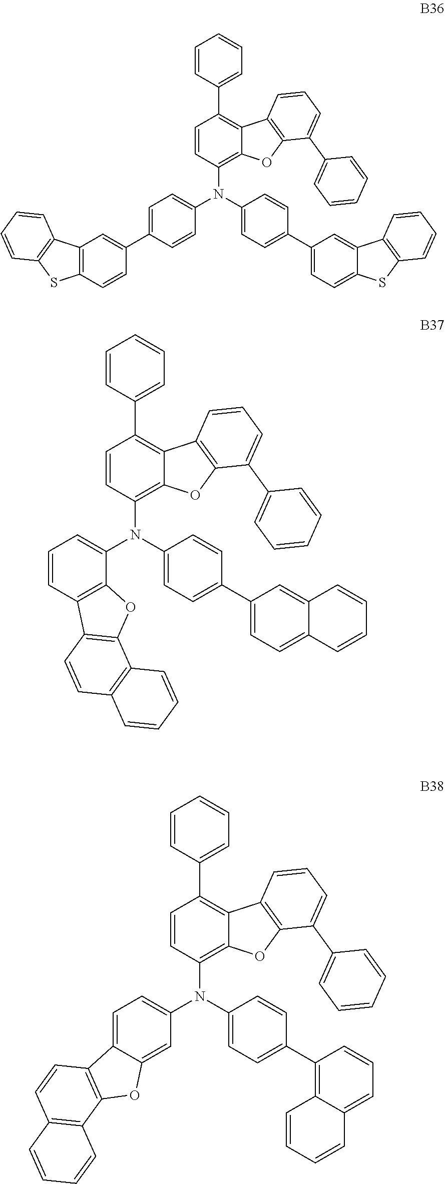 [SMILES: c1ccc(-c2cccc3c2oc2c(N(c4ccc(-c5ccc6sc7ccccc7c6c5)cc4)c4ccc(-c5ccc6sc7ccccc7c6c5)cc4)ccc(-c4ccccc4)c23)cc1 ; c1ccc(-c2cccc3c2oc2c(N(c4ccc(-c5ccc6ccccc6c5)cc4)c4cccc5c4oc4c6ccccc6ccc54)ccc(-c4ccccc4)c23)cc1 ; c1ccc(-c2cccc3c2oc2c(N(c4ccc(-c5cccc6ccccc56)cc4)c4ccc5c(c4)oc4c6ccccc6ccc54)ccc(-c4ccccc4)c23)cc1]

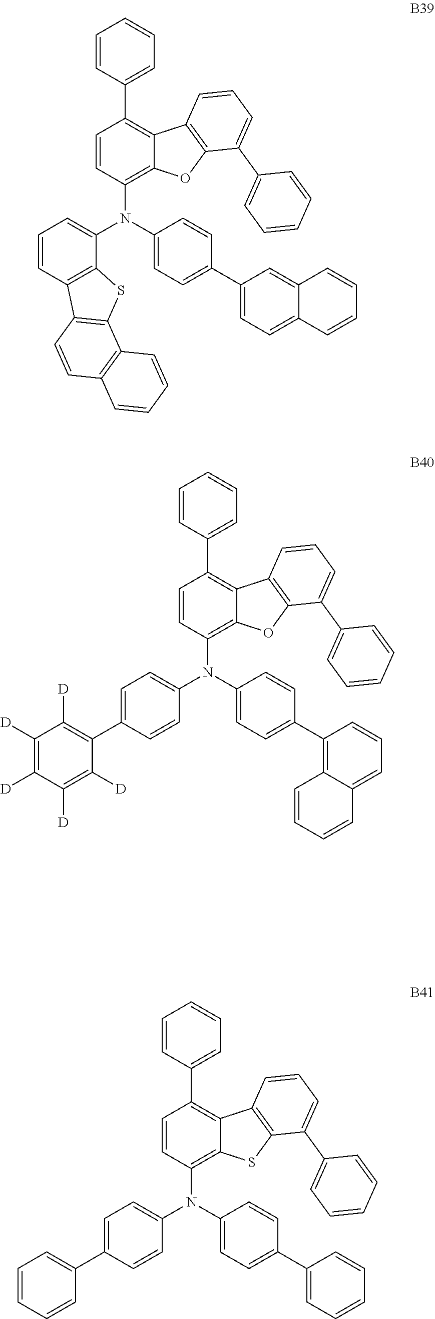 [SMILES: c1ccc(-c2cccc3c2oc2c(N(c4ccc(-c5ccc6ccccc6c5)cc4)c4cccc5c4sc4c6ccccc6ccc54)ccc(-c4ccccc4)c23)cc1 ; [2H]c1c([2H])c([2H])c(-c2ccc(N(c3ccc(-c4cccc5ccccc45)cc3)c3ccc(-c4ccccc4)c4c3oc3c(-c5ccccc5)cccc34)cc2)c([2H])c1[2H] ; c1ccc(-c2ccc(N(c3ccc(-c4ccccc4)cc3)c3ccc(-c4ccccc4)c4c3sc3c(-c5ccccc5)cccc34)cc2)cc1]

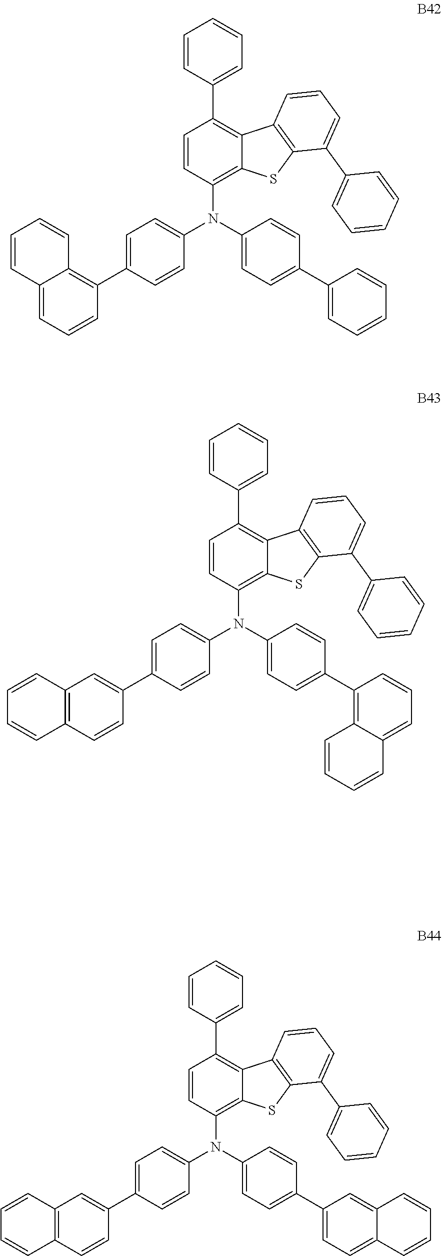 [SMILES: c1ccc(-c2ccc(N(c3ccc(-c4cccc5ccccc45)cc3)c3ccc(-c4ccccc4)c4c3sc3c(-c5ccccc5)cccc34)cc2)cc1 ; c1ccc(-c2cccc3c2sc2c(N(c4ccc(-c5ccc6ccccc6c5)cc4)c4ccc(-c5cccc6ccccc56)cc4)ccc(-c4ccccc4)c23)cc1 ; c1ccc(-c2cccc3c2sc2c(N(c4ccc(-c5ccc6ccccc6c5)cc4)c4ccc(-c5ccc6ccccc6c5)cc4)ccc(-c4ccccc4)c23)cc1]

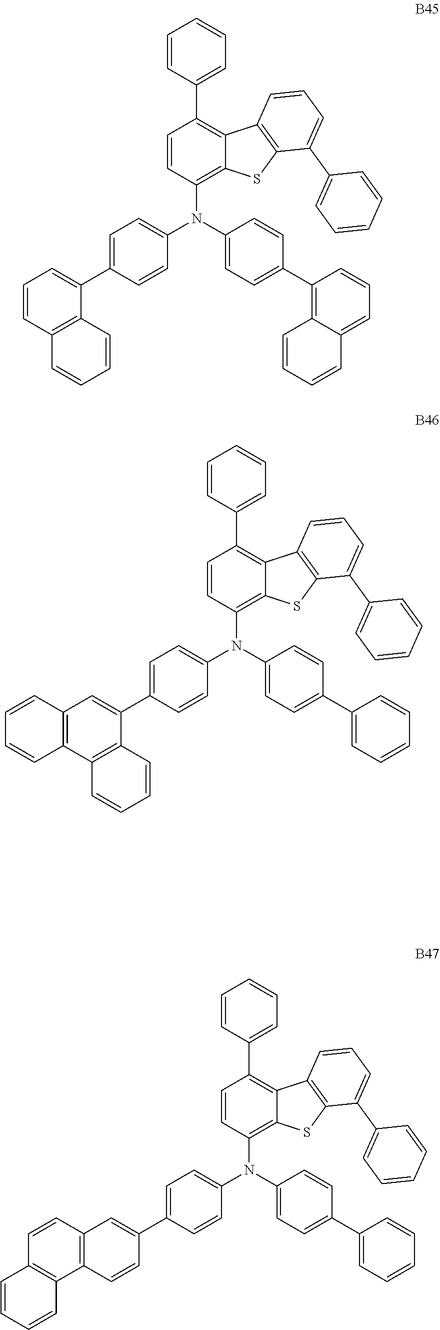 [SMILES: c1ccc(-c2cccc3c2sc2c(N(c4ccc(-c5cccc6ccccc56)cc4)c4ccc(-c5cccc6ccccc56)cc4)ccc(-c4ccccc4)c23)cc1 ; c1ccc(-c2ccc(N(c3ccc(-c4cc5ccccc5c5ccccc45)cc3)c3ccc(-c4ccccc4)c4c3sc3c(-c5ccccc5)cccc34)cc2)cc1 ; c1ccc(-c2ccc(N(c3ccc(-c4ccc5c(ccc6ccccc65)c4)cc3)c3ccc(-c4ccccc4)c4c3sc3c(-c5ccccc5)cccc34)cc2)cc1]

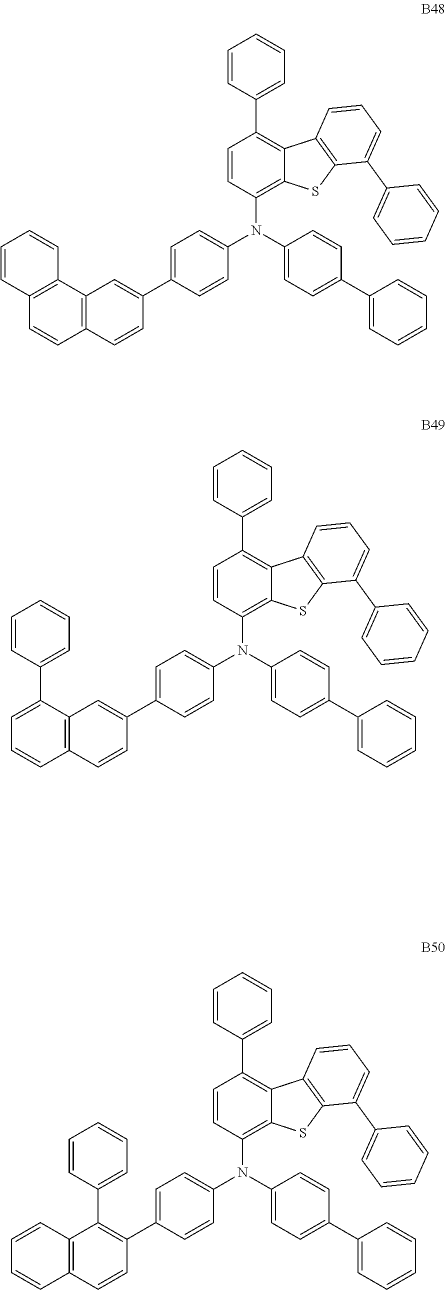 [SMILES: c1ccc(-c2ccc(N(c3ccc(-c4ccc5ccc6ccccc6c5c4)cc3)c3ccc(-c4ccccc4)c4c3sc3c(-c5ccccc5)cccc34)cc2)cc1 ; c1ccc(-c2ccc(N(c3ccc(-c4ccc5cccc(-c6ccccc6)c5c4)cc3)c3ccc(-c4ccccc4)c4c3sc3c(-c5ccccc5)cccc34)cc2)cc1 ; c1ccc(-c2ccc(N(c3ccc(-c4ccc5ccccc5c4-c4ccccc4)cc3)c3ccc(-c4ccccc4)c4c3sc3c(-c5ccccc5)cccc34)cc2)cc1]

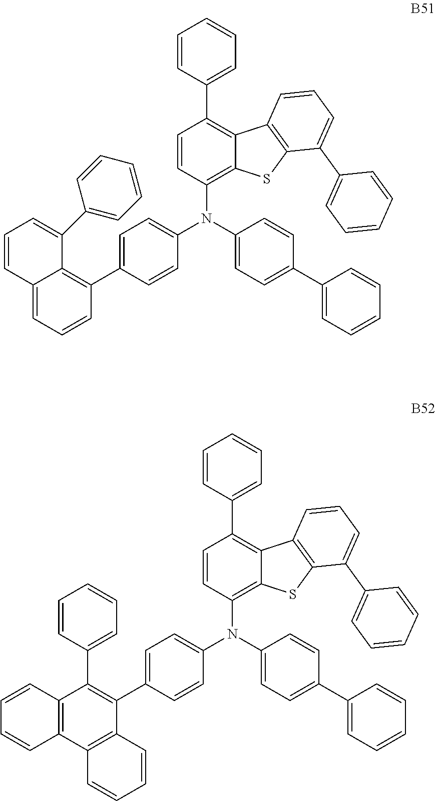 [SMILES: c1ccc(-c2ccc(N(c3ccc(-c4cccc5cccc(-c6ccccc6)c45)cc3)c3ccc(-c4ccccc4)c4c3sc3c(-c5ccccc5)cccc34)cc2)cc1 ; c1ccc(-c2ccc(N(c3ccc(-c4c(-c5ccccc5)c5ccccc5c5ccccc45)cc3)c3ccc(-c4ccccc4)c4c3sc3c(-c5ccccc5)cccc34)cc2)cc1]

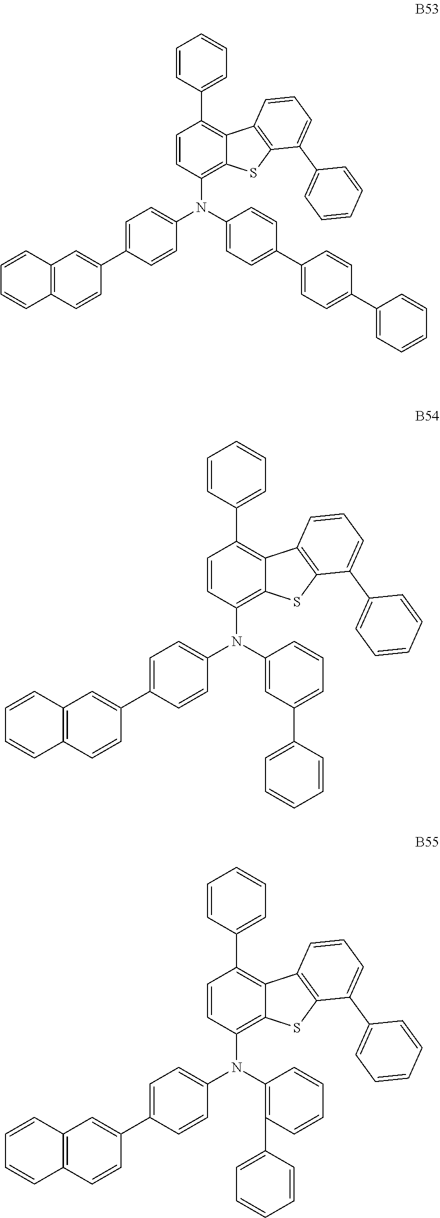 [SMILES: c1ccc(-c2ccc(-c3ccc(N(c4ccc(-c5ccc6ccccc6c5)cc4)c4ccc(-c5ccccc5)c5c4sc4c(-c6ccccc6)cccc45)cc3)cc2)cc1 ; c1ccc(-c2cccc(N(c3ccc(-c4ccc5ccccc5c4)cc3)c3ccc(-c4ccccc4)c4c3sc3c(-c5ccccc5)cccc34)c2)cc1 ; c1ccc(-c2ccccc2N(c2ccc(-c3ccc4ccccc4c3)cc2)c2ccc(-c3ccccc3)c3c2sc2c(-c4ccccc4)cccc23)cc1]

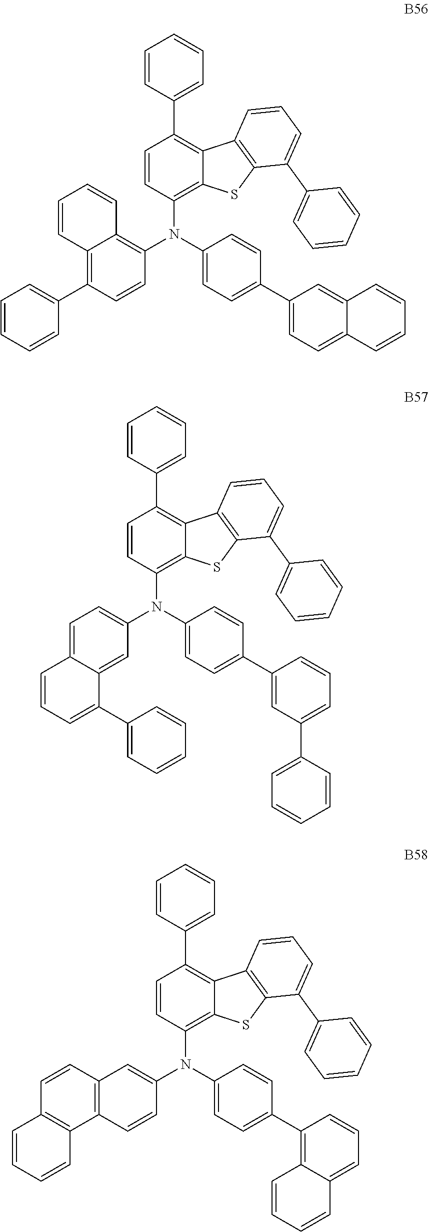 [SMILES: c1ccc(-c2ccc(N(c3ccc(-c4ccc5ccccc5c4)cc3)c3ccc(-c4ccccc4)c4c3sc3c(-c5ccccc5)cccc34)c3ccccc23)cc1 ; c1ccc(-c2cccc(-c3ccc(N(c4ccc5cccc(-c6ccccc6)c5c4)c4ccc(-c5ccccc5)c5c4sc4c(-c6ccccc6)cccc45)cc3)c2)cc1 ; c1ccc(-c2cccc3c2sc2c(N(c4ccc(-c5cccc6ccccc56)cc4)c4ccc5c(ccc6ccccc65)c4)ccc(-c4ccccc4)c23)cc1]

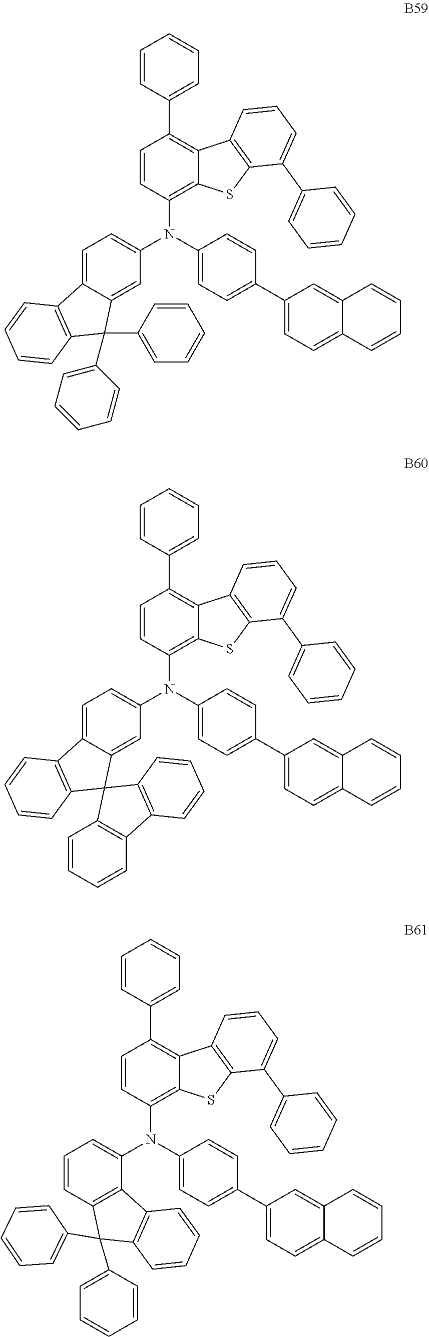 [SMILES: c1ccc(-c2cccc3c2sc2c(N(c4ccc(-c5ccc6ccccc6c5)cc4)c4ccc5c(c4)C(c4ccccc4)(c4ccccc4)c4ccccc4-5)ccc(-c4ccccc4)c23)cc1 ; c1ccc(-c2cccc3c2sc2c(N(c4ccc(-c5ccc6ccccc6c5)cc4)c4ccc5c(c4)C4(c6ccccc6-c6ccccc64)c4ccccc4-5)ccc(-c4ccccc4)c23)cc1 ; c1ccc(-c2cccc3c2sc2c(N(c4ccc(-c5ccc6ccccc6c5)cc4)c4cccc5c4-c4ccccc4C5(c4ccccc4)c4ccccc4)ccc(-c4ccccc4)c23)cc1]

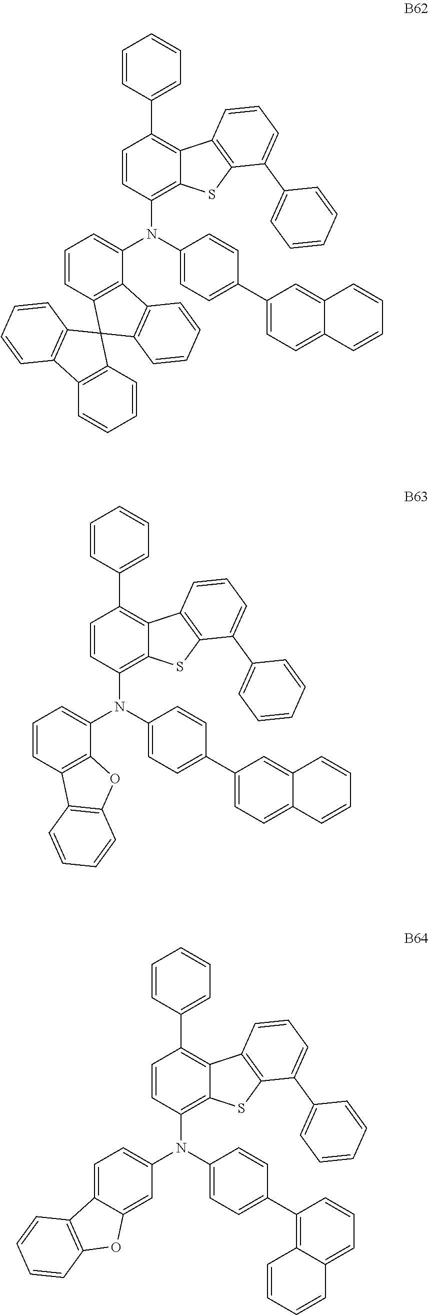 [SMILES: c1ccc(-c2cccc3c2sc2c(N(c4ccc(-c5ccc6ccccc6c5)cc4)c4cccc5c4-c4ccccc4C54c5ccccc5-c5ccccc54)ccc(-c4ccccc4)c23)cc1 ; c1ccc(-c2cccc3c2sc2c(N(c4ccc(-c5ccc6ccccc6c5)cc4)c4cccc5c4oc4ccccc45)ccc(-c4ccccc4)c23)cc1 ; c1ccc(-c2cccc3c2sc2c(N(c4ccc(-c5cccc6ccccc56)cc4)c4ccc5c(c4)oc4ccccc45)ccc(-c4ccccc4)c23)cc1]

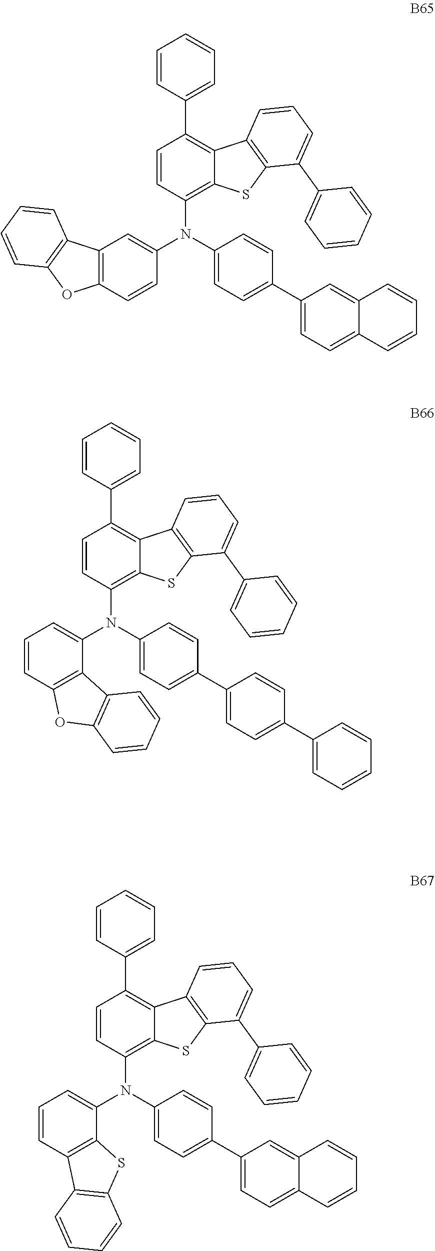 [SMILES: c1ccc(-c2cccc3c2sc2c(N(c4ccc(-c5ccc6ccccc6c5)cc4)c4ccc5oc6ccccc6c5c4)ccc(-c4ccccc4)c23)cc1 ; c1ccc(-c2ccc(-c3ccc(N(c4ccc(-c5ccccc5)c5c4sc4c(-c6ccccc6)cccc45)c4cccc5oc6ccccc6c45)cc3)cc2)cc1 ; c1ccc(-c2cccc3c2sc2c(N(c4ccc(-c5ccc6ccccc6c5)cc4)c4cccc5c4sc4ccccc45)ccc(-c4ccccc4)c23)cc1]

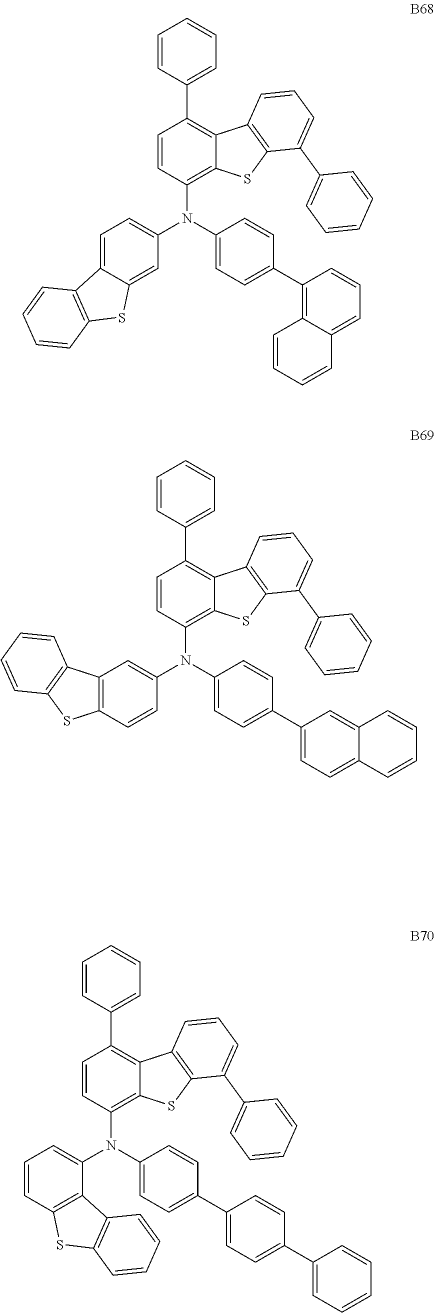 [SMILES: c1ccc(-c2cccc3c2sc2c(N(c4ccc(-c5cccc6ccccc56)cc4)c4ccc5c(c4)sc4ccccc45)ccc(-c4ccccc4)c23)cc1 ; c1ccc(-c2cccc3c2sc2c(N(c4ccc(-c5ccc6ccccc6c5)cc4)c4ccc5sc6ccccc6c5c4)ccc(-c4ccccc4)c23)cc1 ; c1ccc(-c2ccc(-c3ccc(N(c4ccc(-c5ccccc5)c5c4sc4c(-c6ccccc6)cccc45)c4cccc5sc6ccccc6c45)cc3)cc2)cc1]

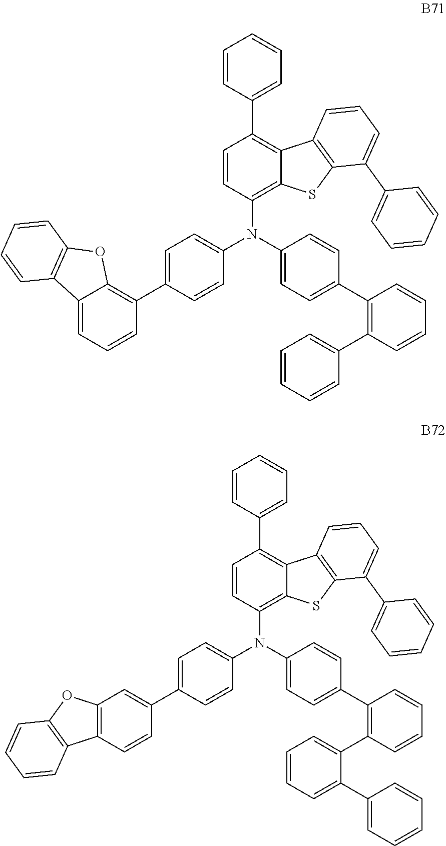 [SMILES: c1ccc(-c2ccccc2-c2ccc(N(c3ccc(-c4cccc5c4oc4ccccc45)cc3)c3ccc(-c4ccccc4)c4c3sc3c(-c5ccccc5)cccc34)cc2)cc1 ; c1ccc(-c2ccccc2-c2ccccc2-c2ccc(N(c3ccc(-c4ccc5c(c4)oc4ccccc45)cc3)c3ccc(-c4ccccc4)c4c3sc3c(-c5ccccc5)cccc34)cc2)cc1]

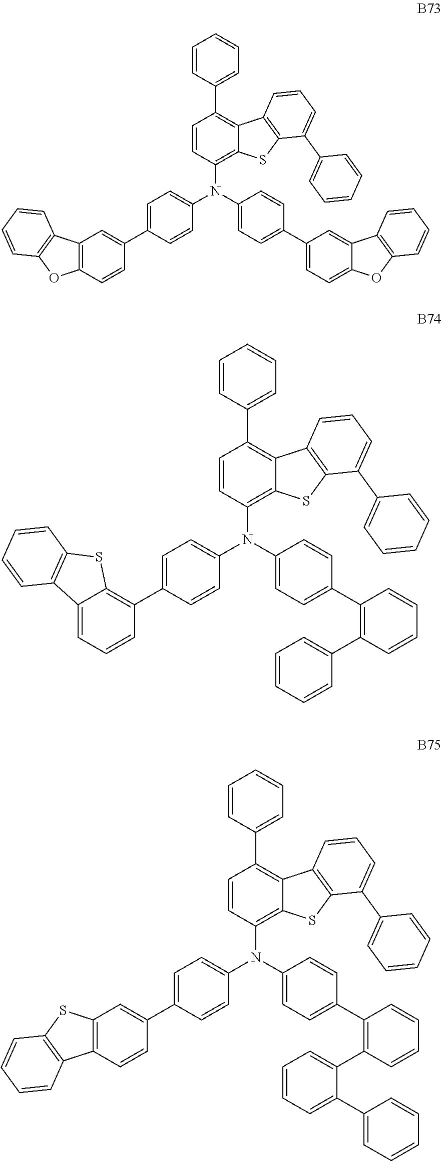 [SMILES: c1ccc(-c2cccc3c2sc2c(N(c4ccc(-c5ccc6oc7ccccc7c6c5)cc4)c4ccc(-c5ccc6oc7ccccc7c6c5)cc4)ccc(-c4ccccc4)c23)cc1 ; c1ccc(-c2ccccc2-c2ccc(N(c3ccc(-c4cccc5c4sc4ccccc45)cc3)c3ccc(-c4ccccc4)c4c3sc3c(-c5ccccc5)cccc34)cc2)cc1 ; c1ccc(-c2ccccc2-c2ccccc2-c2ccc(N(c3ccc(-c4ccc5c(c4)sc4ccccc45)cc3)c3ccc(-c4ccccc4)c4c3sc3c(-c5ccccc5)cccc34)cc2)cc1]

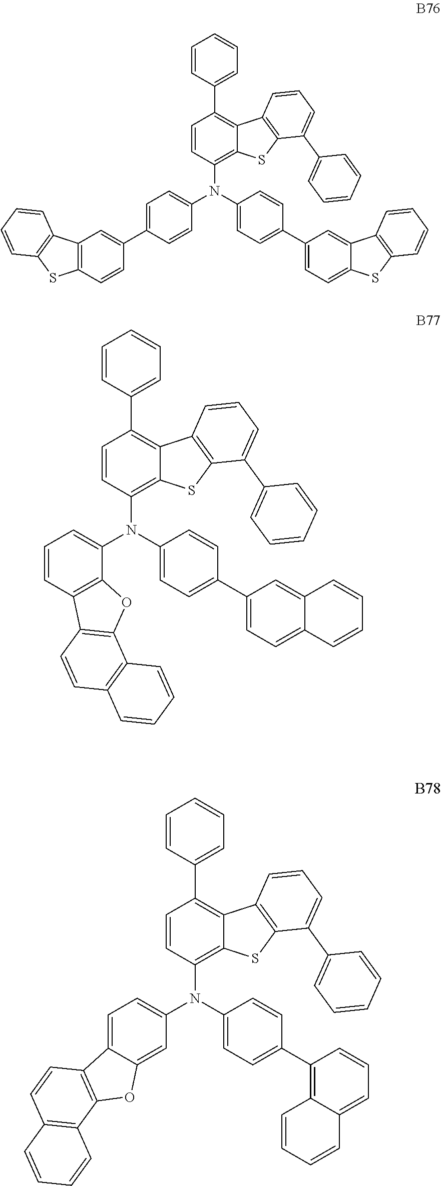 [SMILES: c1ccc(-c2cccc3c2sc2c(N(c4ccc(-c5ccc6sc7ccccc7c6c5)cc4)c4ccc(-c5ccc6sc7ccccc7c6c5)cc4)ccc(-c4ccccc4)c23)cc1 ; c1ccc(-c2cccc3c2sc2c(N(c4ccc(-c5ccc6ccccc6c5)cc4)c4cccc5c4oc4c6ccccc6ccc54)ccc(-c4ccccc4)c23)cc1 ; c1ccc(-c2cccc3c2sc2c(N(c4ccc(-c5cccc6ccccc56)cc4)c4ccc5c(c4)oc4c6ccccc6ccc54)ccc(-c4ccccc4)c23)cc1]

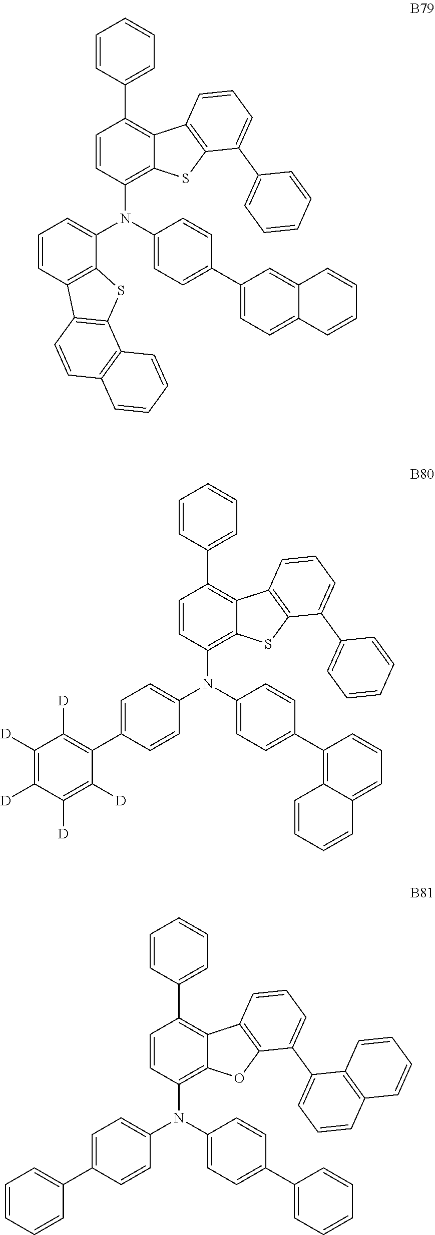 [SMILES: c1ccc(-c2cccc3c2sc2c(N(c4ccc(-c5ccc6ccccc6c5)cc4)c4cccc5c4sc4c6ccccc6ccc54)ccc(-c4ccccc4)c23)cc1 ; [2H]c1c([2H])c([2H])c(-c2ccc(N(c3ccc(-c4cccc5ccccc45)cc3)c3ccc(-c4ccccc4)c4c3sc3c(-c5ccccc5)cccc34)cc2)c([2H])c1[2H] ; c1ccc(-c2ccc(N(c3ccc(-c4ccccc4)cc3)c3ccc(-c4ccccc4)c4c3oc3c(-c5cccc6ccccc56)cccc34)cc2)cc1]

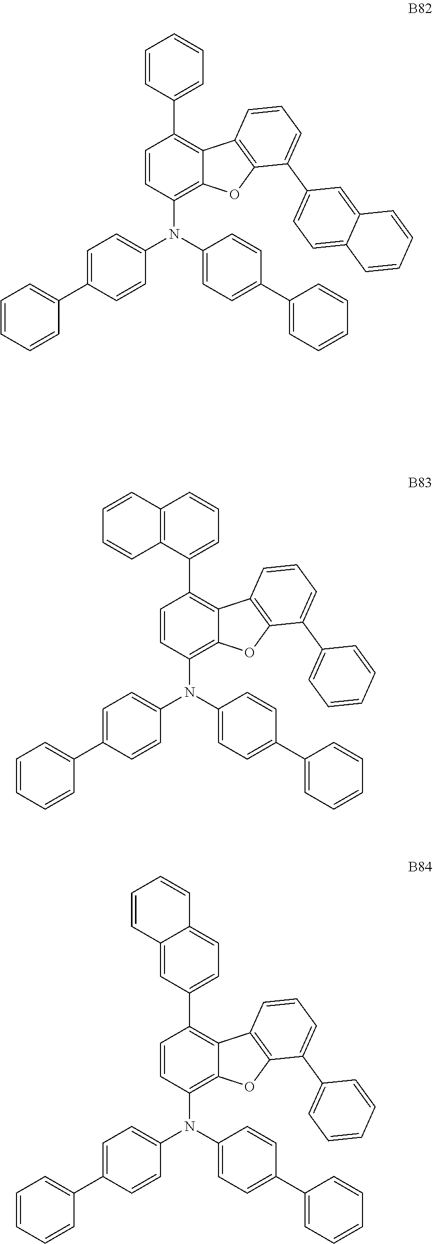 [SMILES: c1ccc(-c2ccc(N(c3ccc(-c4ccccc4)cc3)c3ccc(-c4ccccc4)c4c3oc3c(-c5ccc6ccccc6c5)cccc34)cc2)cc1 ; c1ccc(-c2ccc(N(c3ccc(-c4ccccc4)cc3)c3ccc(-c4cccc5ccccc45)c4c3oc3c(-c5ccccc5)cccc34)cc2)cc1 ; c1ccc(-c2ccc(N(c3ccc(-c4ccccc4)cc3)c3ccc(-c4ccc5ccccc5c4)c4c3oc3c(-c5ccccc5)cccc34)cc2)cc1]

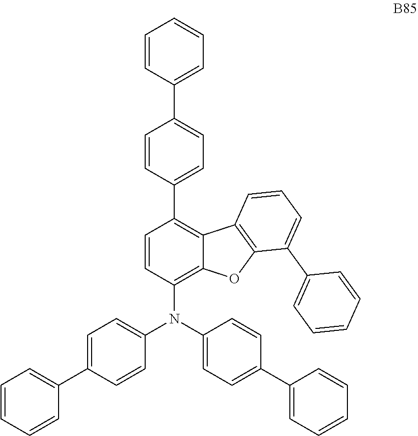 [SMILES: c1ccc(-c2ccc(-c3ccc(N(c4ccc(-c5ccccc5)cc4)c4ccc(-c5ccccc5)cc4)c4oc5c(-c6ccccc6)cccc5c34)cc2)cc1]

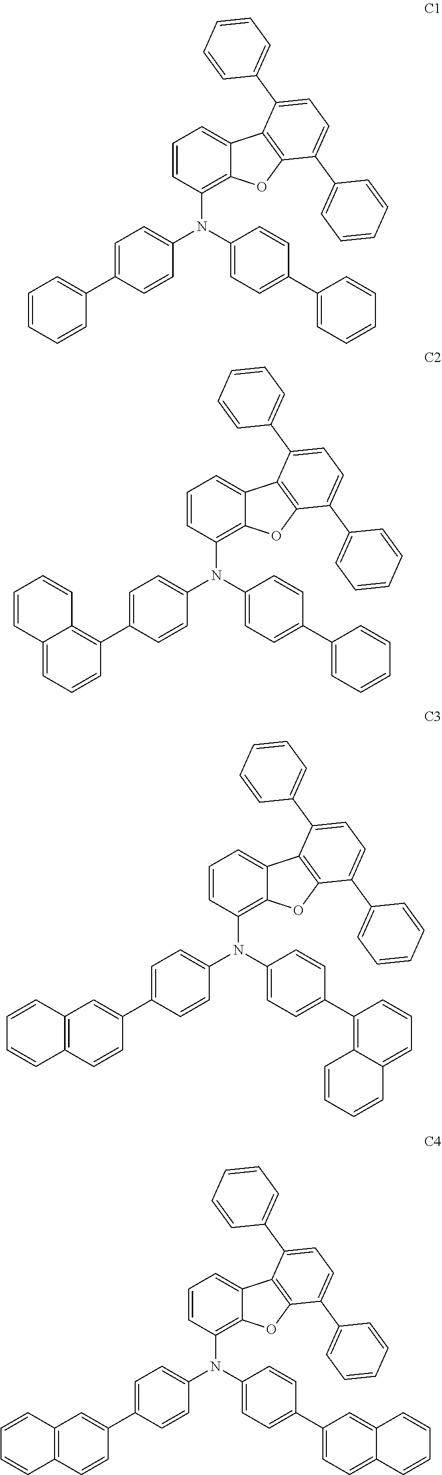 [SMILES: c1ccc(-c2ccc(N(c3ccc(-c4ccccc4)cc3)c3cccc4c3oc3c(-c5ccccc5)ccc(-c5ccccc5)c34)cc2)cc1 ; c1ccc(-c2ccc(N(c3ccc(-c4cccc5ccccc45)cc3)c3cccc4c3oc3c(-c5ccccc5)ccc(-c5ccccc5)c34)cc2)cc1 ; c1ccc(-c2ccc(-c3ccccc3)c3c2oc2c(N(c4ccc(-c5ccc6ccccc6c5)cc4)c4ccc(-c5cccc6ccccc56)cc4)cccc23)cc1 ; c1ccc(-c2ccc(-c3ccccc3)c3c2oc2c(N(c4ccc(-c5ccc6ccccc6c5)cc4)c4ccc(-c5ccc6ccccc6c5)cc4)cccc23)cc1]

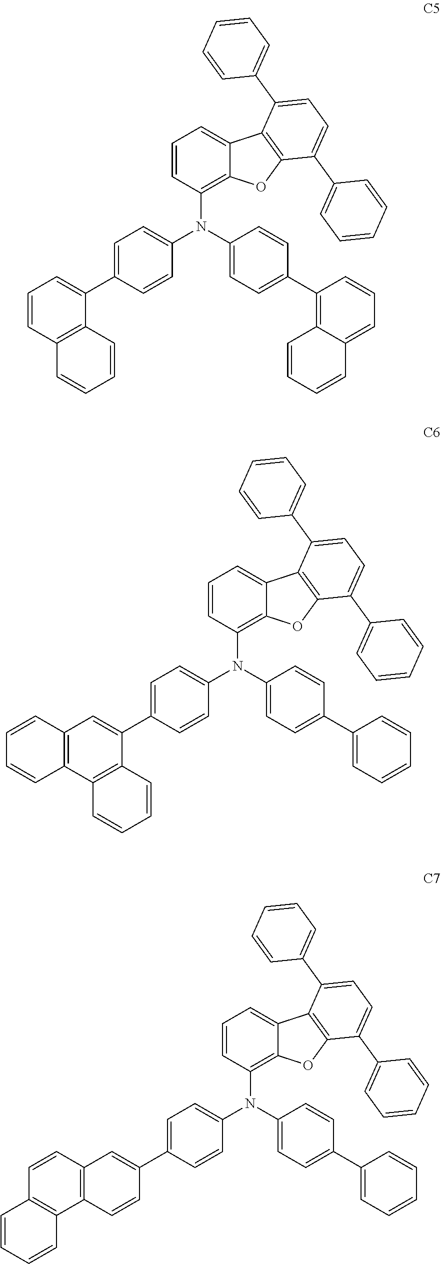 [SMILES: c1ccc(-c2ccc(-c3ccccc3)c3c2oc2c(N(c4ccc(-c5cccc6ccccc56)cc4)c4ccc(-c5cccc6ccccc56)cc4)cccc23)cc1 ; c1ccc(-c2ccc(N(c3ccc(-c4cc5ccccc5c5ccccc45)cc3)c3cccc4c3oc3c(-c5ccccc5)ccc(-c5ccccc5)c34)cc2)cc1 ; c1ccc(-c2ccc(N(c3ccc(-c4ccc5c(ccc6ccccc65)c4)cc3)c3cccc4c3oc3c(-c5ccccc5)ccc(-c5ccccc5)c34)cc2)cc1]

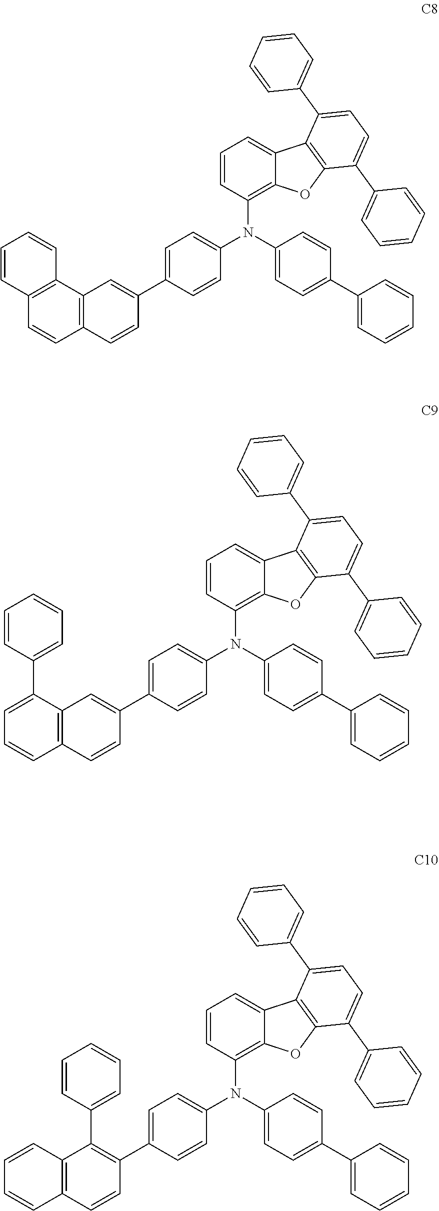 [SMILES: c1ccc(-c2ccc(N(c3ccc(-c4ccc5ccc6ccccc6c5c4)cc3)c3cccc4c3oc3c(-c5ccccc5)ccc(-c5ccccc5)c34)cc2)cc1 ; c1ccc(-c2ccc(N(c3ccc(-c4ccc5cccc(-c6ccccc6)c5c4)cc3)c3cccc4c3oc3c(-c5ccccc5)ccc(-c5ccccc5)c34)cc2)cc1 ; c1ccc(-c2ccc(N(c3ccc(-c4ccc5ccccc5c4-c4ccccc4)cc3)c3cccc4c3oc3c(-c5ccccc5)ccc(-c5ccccc5)c34)cc2)cc1]

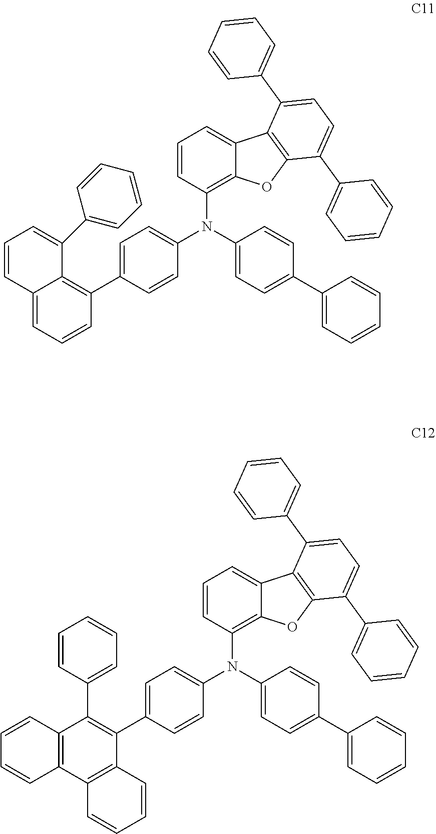 [SMILES: c1ccc(-c2ccc(N(c3ccc(-c4cccc5cccc(-c6ccccc6)c45)cc3)c3cccc4c3oc3c(-c5ccccc5)ccc(-c5ccccc5)c34)cc2)cc1 ; c1ccc(-c2ccc(N(c3ccc(-c4c(-c5ccccc5)c5ccccc5c5ccccc45)cc3)c3cccc4c3oc3c(-c5ccccc5)ccc(-c5ccccc5)c34)cc2)cc1]

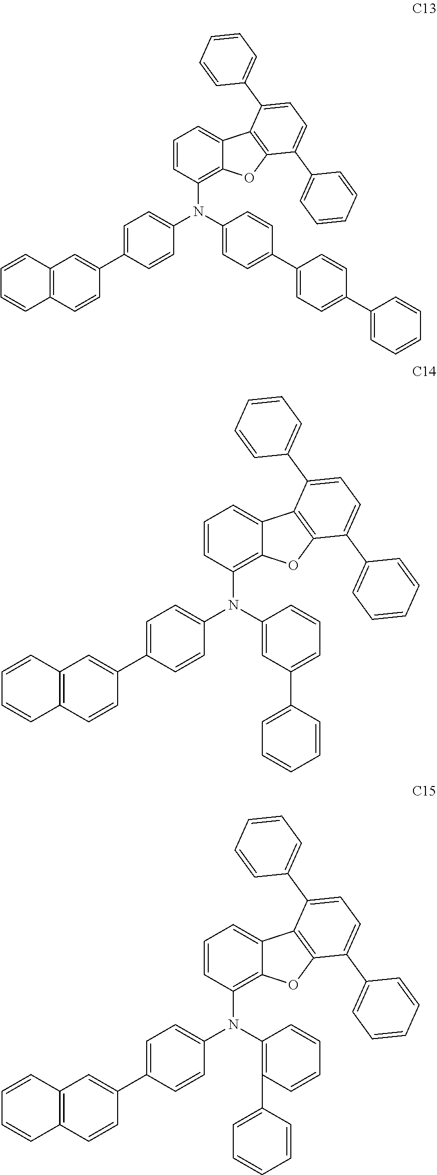

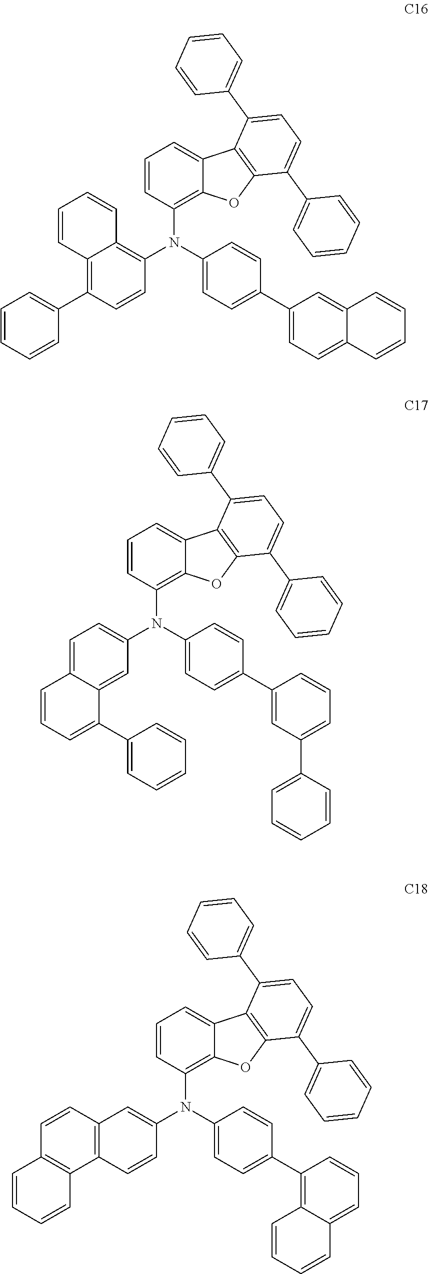 [SMILES: c1ccc(-c2ccc(N(c3ccc(-c4ccc5ccccc5c4)cc3)c3cccc4c3oc3c(-c5ccccc5)ccc(-c5ccccc5)c34)c3ccccc23)cc1 ; c1ccc(-c2cccc(-c3ccc(N(c4ccc5cccc(-c6ccccc6)c5c4)c4cccc5c4oc4c(-c6ccccc6)ccc(-c6ccccc6)c45)cc3)c2)cc1 ; c1ccc(-c2ccc(-c3ccccc3)c3c2oc2c(N(c4ccc(-c5cccc6ccccc56)cc4)c4ccc5c(ccc6ccccc65)c4)cccc23)cc1]

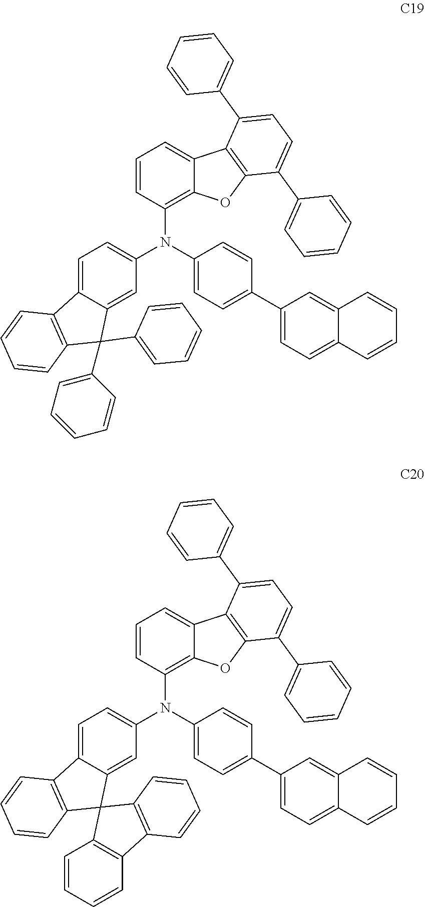 [SMILES: c1ccc(-c2ccc(-c3ccccc3)c3c2oc2c(N(c4ccc(-c5ccc6ccccc6c5)cc4)c4ccc5c(c4)C(c4ccccc4)(c4ccccc4)c4ccccc4-5)cccc23)cc1 ; c1ccc(-c2ccc(-c3ccccc3)c3c2oc2c(N(c4ccc(-c5ccc6ccccc6c5)cc4)c4ccc5c(c4)C4(c6ccccc6-c6ccccc64)c4ccccc4-5)cccc23)cc1]

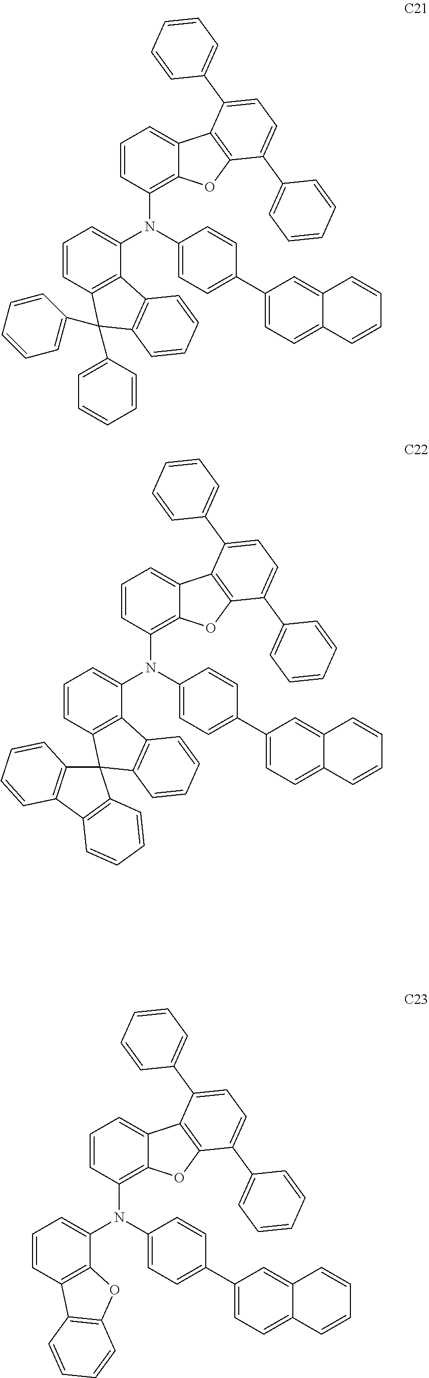 [SMILES: c1ccc(-c2ccc(-c3ccccc3)c3c2oc2c(N(c4ccc(-c5ccc6ccccc6c5)cc4)c4cccc5c4-c4ccccc4C5(c4ccccc4)c4ccccc4)cccc23)cc1 ; c1ccc(-c2ccc(-c3ccccc3)c3c2oc2c(N(c4ccc(-c5ccc6ccccc6c5)cc4)c4cccc5c4-c4ccccc4C54c5ccccc5-c5ccccc54)cccc23)cc1 ; c1ccc(-c2ccc(-c3ccccc3)c3c2oc2c(N(c4ccc(-c5ccc6ccccc6c5)cc4)c4cccc5c4oc4ccccc45)cccc23)cc1]

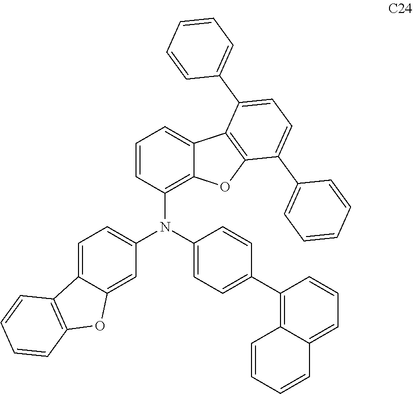 [SMILES: c1ccc(-c2ccc(-c3ccccc3)c3c2oc2c(N(c4ccc(-c5cccc6ccccc56)cc4)c4ccc5c(c4)oc4ccccc45)cccc23)cc1]

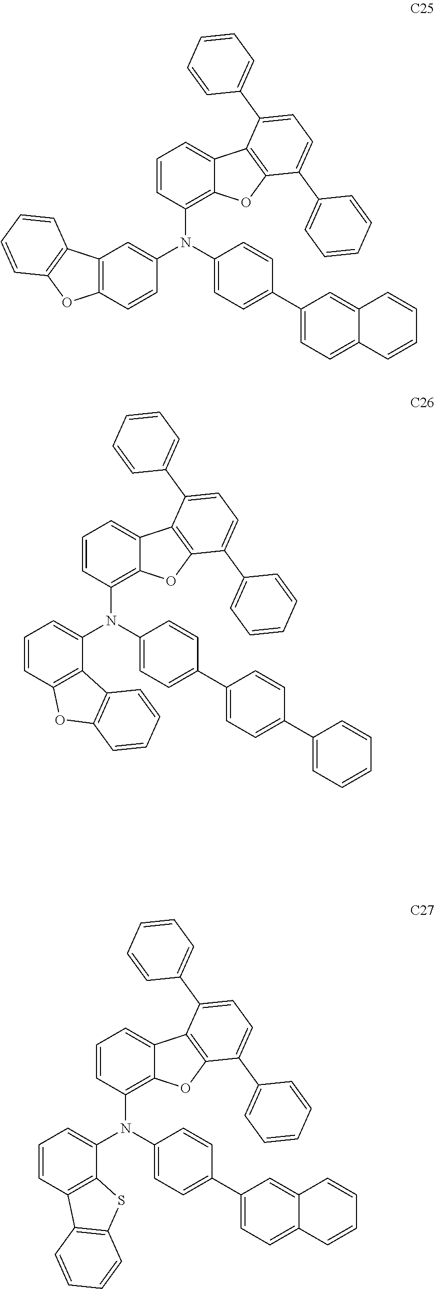 [SMILES: c1ccc(-c2ccc(-c3ccccc3)c3c2oc2c(N(c4ccc(-c5ccc6ccccc6c5)cc4)c4ccc5oc6ccccc6c5c4)cccc23)cc1 ; c1ccc(-c2ccc(-c3ccc(N(c4cccc5c4oc4c(-c6ccccc6)ccc(-c6ccccc6)c45)c4cccc5oc6ccccc6c45)cc3)cc2)cc1 ; c1ccc(-c2ccc(-c3ccccc3)c3c2oc2c(N(c4ccc(-c5ccc6ccccc6c5)cc4)c4cccc5c4sc4ccccc45)cccc23)cc1]

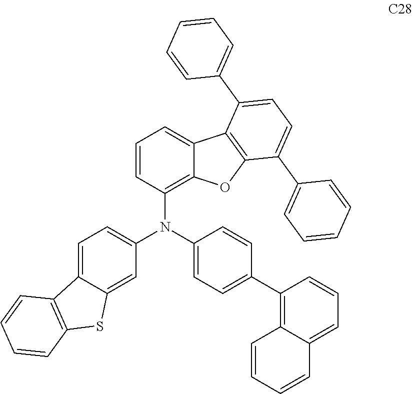 [SMILES: c1ccc(-c2ccc(-c3ccccc3)c3c2oc2c(N(c4ccc(-c5cccc6ccccc56)cc4)c4ccc5c(c4)sc4ccccc45)cccc23)cc1]

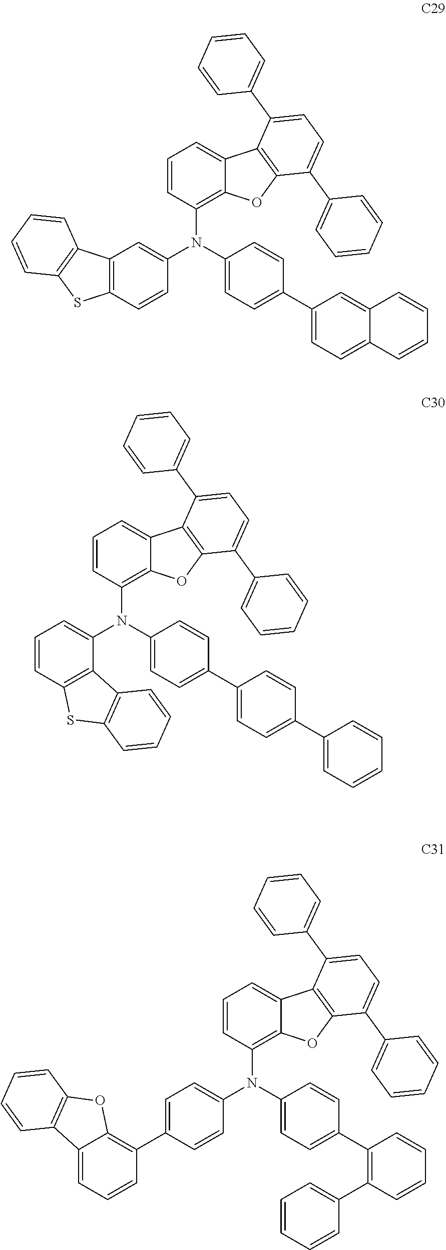 [SMILES: c1ccc(-c2ccc(-c3ccccc3)c3c2oc2c(N(c4ccc(-c5ccc6ccccc6c5)cc4)c4ccc5sc6ccccc6c5c4)cccc23)cc1 ; c1ccc(-c2ccc(-c3ccc(N(c4cccc5c4oc4c(-c6ccccc6)ccc(-c6ccccc6)c45)c4cccc5sc6ccccc6c45)cc3)cc2)cc1 ; c1ccc(-c2ccccc2-c2ccc(N(c3ccc(-c4cccc5c4oc4ccccc45)cc3)c3cccc4c3oc3c(-c5ccccc5)ccc(-c5ccccc5)c34)cc2)cc1]

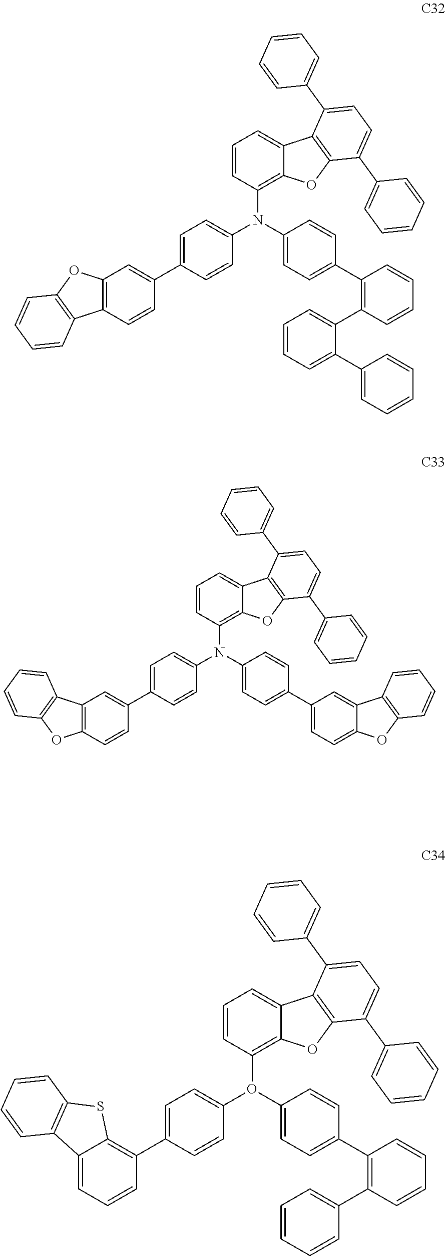

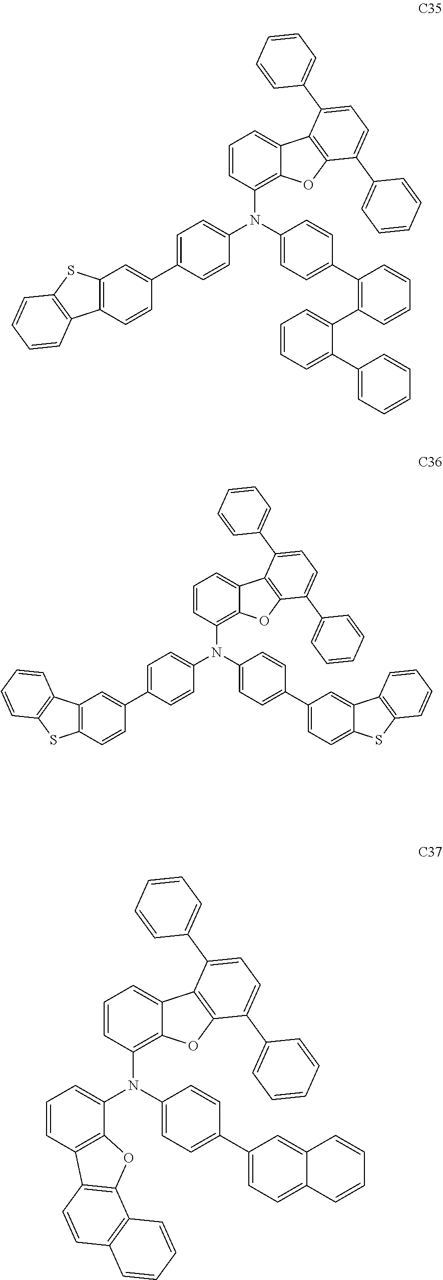 [SMILES: c1ccc(-c2ccccc2-c2ccccc2-c2ccc(N(c3ccc(-c4ccc5c(c4)sc4ccccc45)cc3)c3cccc4c3oc3c(-c5ccccc5)ccc(-c5ccccc5)c34)cc2)cc1 ; c1ccc(-c2ccc(-c3ccccc3)c3c2oc2c(N(c4ccc(-c5ccc6sc7ccccc7c6c5)cc4)c4ccc(-c5ccc6sc7ccccc7c6c5)cc4)cccc23)cc1 ; c1ccc(-c2ccc(-c3ccccc3)c3c2oc2c(N(c4ccc(-c5ccc6ccccc6c5)cc4)c4cccc5c4oc4c6ccccc6ccc54)cccc23)cc1]

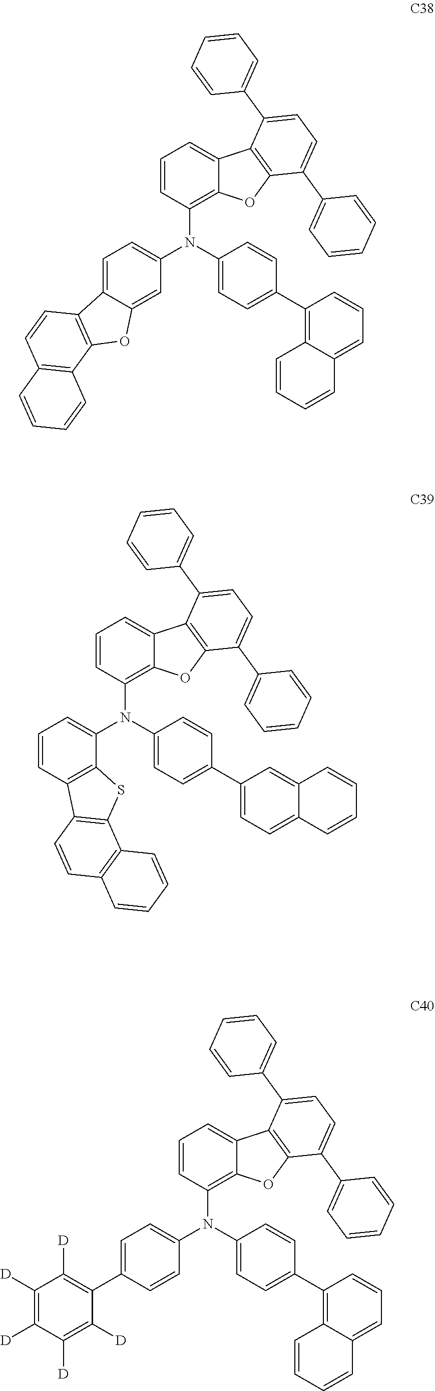 [SMILES: c1ccc(-c2ccc(-c3ccccc3)c3c2oc2c(N(c4ccc(-c5cccc6ccccc56)cc4)c4ccc5c(c4)oc4c6ccccc6ccc54)cccc23)cc1 ; c1ccc(-c2ccc(-c3ccccc3)c3c2oc2c(N(c4ccc(-c5ccc6ccccc6c5)cc4)c4cccc5c4sc4c6ccccc6ccc54)cccc23)cc1 ; [2H]c1c([2H])c([2H])c(-c2ccc(N(c3ccc(-c4cccc5ccccc45)cc3)c3cccc4c3oc3c(-c5ccccc5)ccc(-c5ccccc5)c34)cc2)c([2H])c1[2H]]

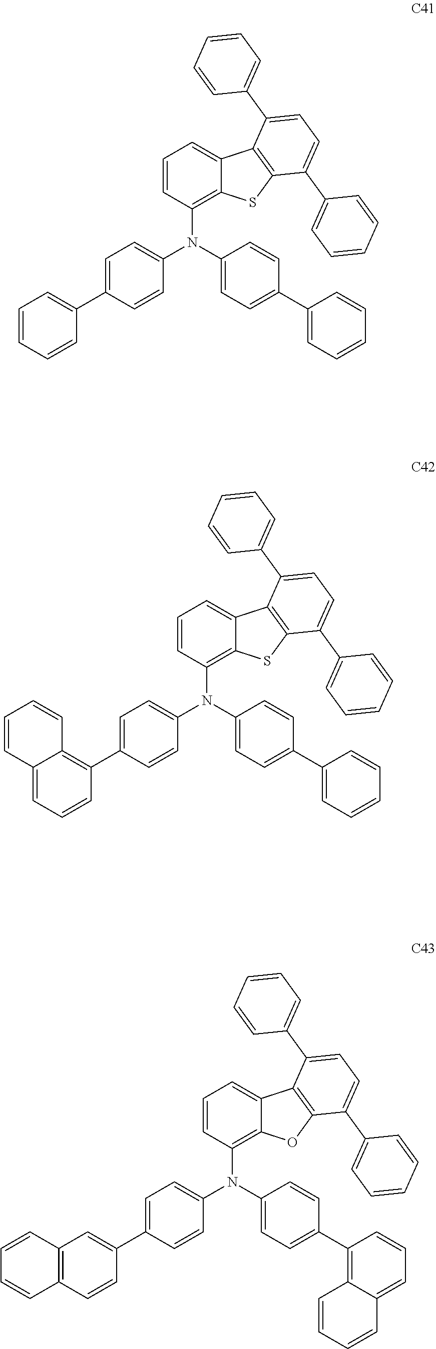 [SMILES: c1ccc(-c2ccc(N(c3ccc(-c4ccccc4)cc3)c3cccc4c3sc3c(-c5ccccc5)ccc(-c5ccccc5)c34)cc2)cc1 ; c1ccc(-c2ccc(N(c3ccc(-c4cccc5ccccc45)cc3)c3cccc4c3sc3c(-c5ccccc5)ccc(-c5ccccc5)c34)cc2)cc1 ; c1ccc(-c2ccc(-c3ccccc3)c3c2oc2c(N(c4ccc(-c5ccc6ccccc6c5)cc4)c4ccc(-c5cccc6ccccc56)cc4)cccc23)cc1]

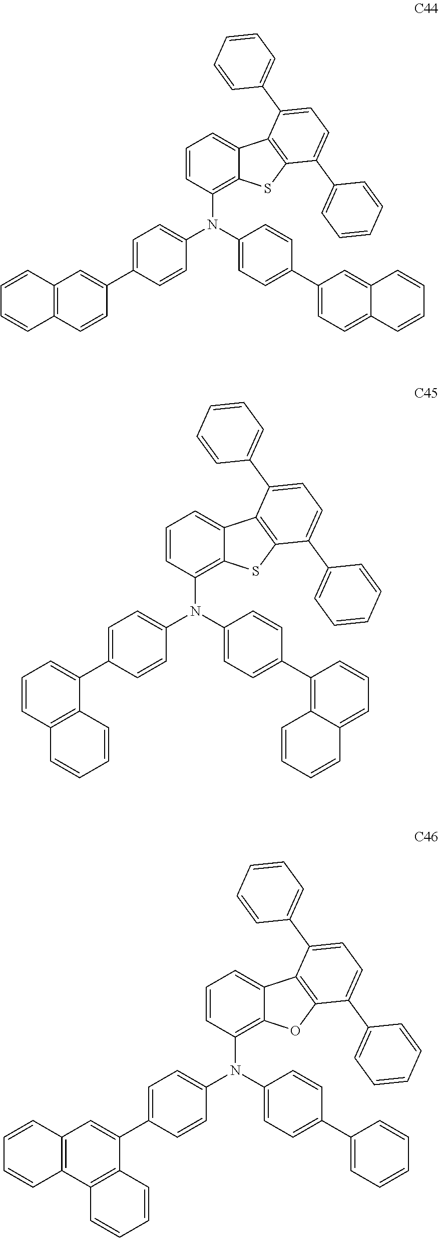 [SMILES: c1ccc(-c2ccc(-c3ccccc3)c3c2sc2c(N(c4ccc(-c5ccc6ccccc6c5)cc4)c4ccc(-c5ccc6ccccc6c5)cc4)cccc23)cc1 ; c1ccc(-c2ccc(-c3ccccc3)c3c2sc2c(N(c4ccc(-c5cccc6ccccc56)cc4)c4ccc(-c5cccc6ccccc56)cc4)cccc23)cc1 ; c1ccc(-c2ccc(N(c3ccc(-c4cc5ccccc5c5ccccc45)cc3)c3cccc4c3oc3c(-c5ccccc5)ccc(-c5ccccc5)c34)cc2)cc1]

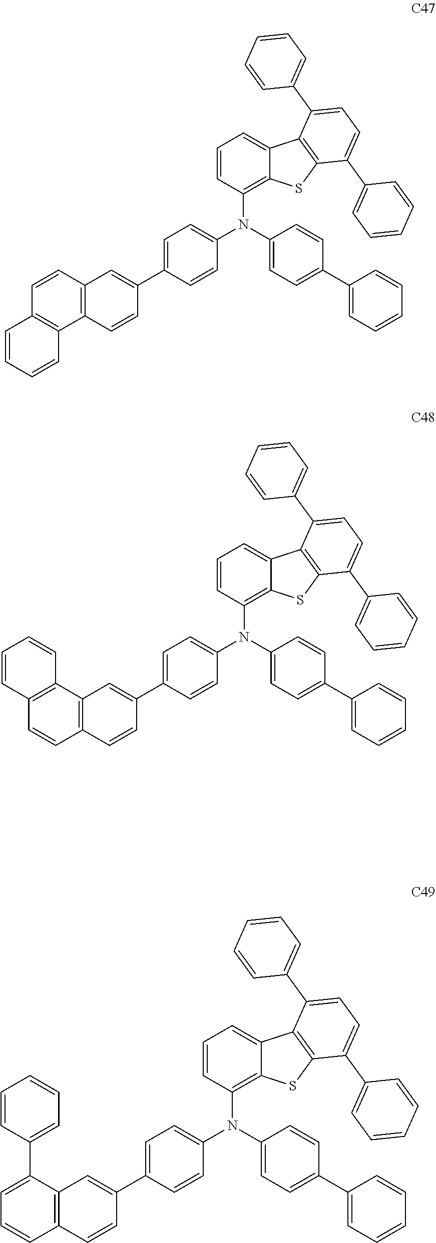 [SMILES: c1ccc(-c2ccc(N(c3ccc(-c4ccc5c(ccc6ccccc65)c4)cc3)c3cccc4c3sc3c(-c5ccccc5)ccc(-c5ccccc5)c34)cc2)cc1 ; c1ccc(-c2ccc(N(c3ccc(-c4ccc5ccc6ccccc6c5c4)cc3)c3cccc4c3sc3c(-c5ccccc5)ccc(-c5ccccc5)c34)cc2)cc1 ; c1ccc(-c2ccc(N(c3ccc(-c4ccc5cccc(-c6ccccc6)c5c4)cc3)c3cccc4c3sc3c(-c5ccccc5)ccc(-c5ccccc5)c34)cc2)cc1]

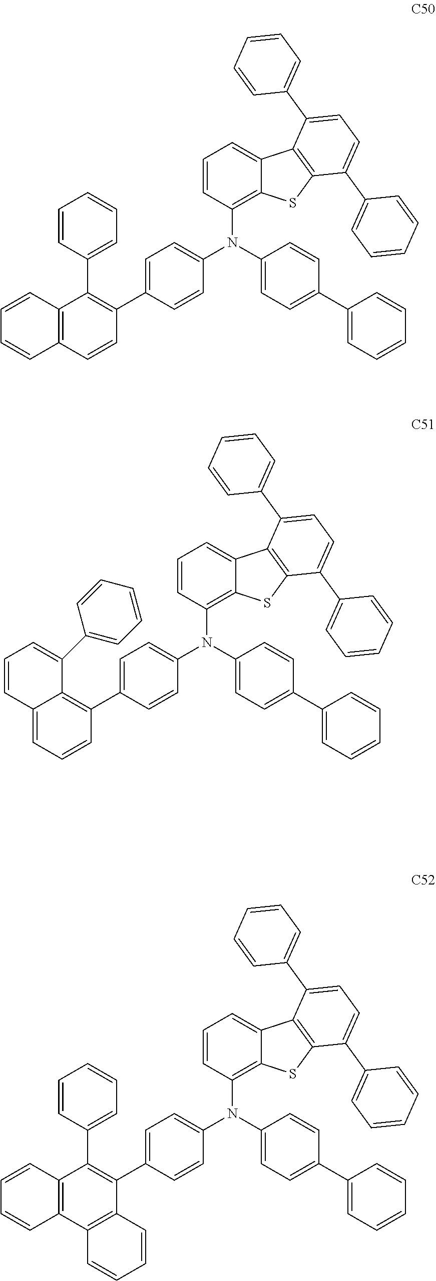 [SMILES: c1ccc(-c2ccc(N(c3ccc(-c4ccc5ccccc5c4-c4ccccc4)cc3)c3cccc4c3sc3c(-c5ccccc5)ccc(-c5ccccc5)c34)cc2)cc1 ; c1ccc(-c2ccc(N(c3ccc(-c4cccc5cccc(-c6ccccc6)c45)cc3)c3cccc4c3sc3c(-c5ccccc5)ccc(-c5ccccc5)c34)cc2)cc1 ; c1ccc(-c2ccc(N(c3ccc(-c4c(-c5ccccc5)c5ccccc5c5ccccc45)cc3)c3cccc4c3sc3c(-c5ccccc5)ccc(-c5ccccc5)c34)cc2)cc1]

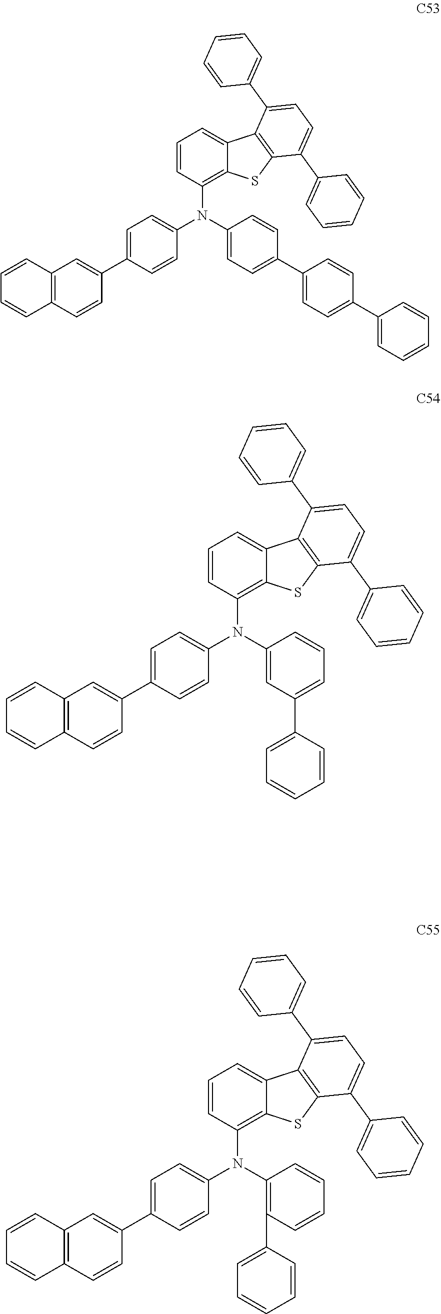 [SMILES: c1ccc(-c2ccc(-c3ccc(N(c4ccc(-c5ccc6ccccc6c5)cc4)c4cccc5c4sc4c(-c6ccccc6)ccc(-c6ccccc6)c45)cc3)cc2)cc1 ; c1ccc(-c2cccc(N(c3ccc(-c4ccc5ccccc5c4)cc3)c3cccc4c3sc3c(-c5ccccc5)ccc(-c5ccccc5)c34)c2)cc1 ; c1ccc(-c2ccccc2N(c2ccc(-c3ccc4ccccc4c3)cc2)c2cccc3c2sc2c(-c4ccccc4)ccc(-c4ccccc4)c23)cc1]

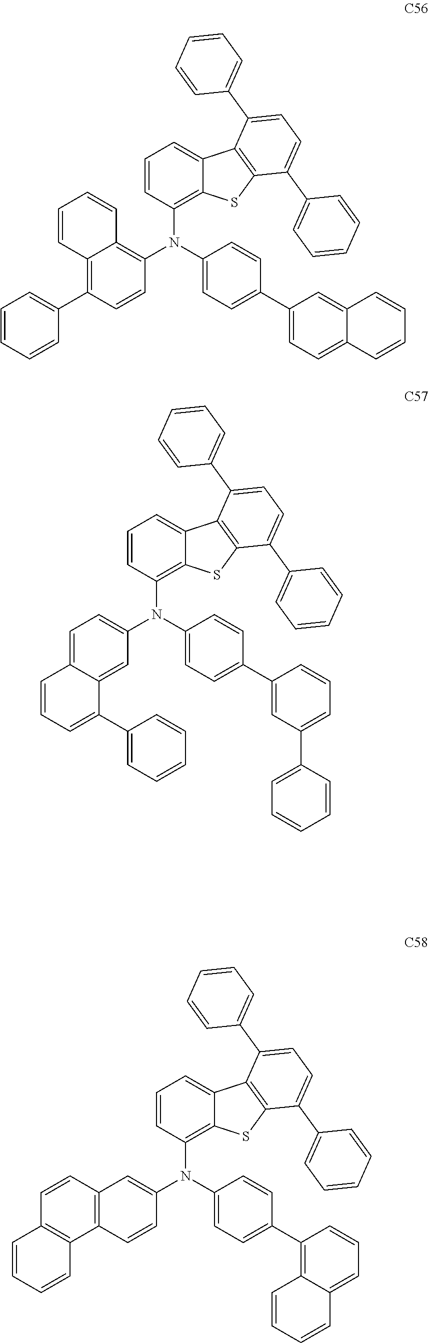 [SMILES: c1ccc(-c2ccc(N(c3ccc(-c4ccc5ccccc5c4)cc3)c3cccc4c3sc3c(-c5ccccc5)ccc(-c5ccccc5)c34)c3ccccc23)cc1 ; c1ccc(-c2cccc(-c3ccc(N(c4ccc5cccc(-c6ccccc6)c5c4)c4cccc5c4sc4c(-c6ccccc6)ccc(-c6ccccc6)c45)cc3)c2)cc1 ; c1ccc(-c2ccc(-c3ccccc3)c3c2sc2c(N(c4ccc(-c5cccc6ccccc56)cc4)c4ccc5c(ccc6ccccc65)c4)cccc23)cc1]

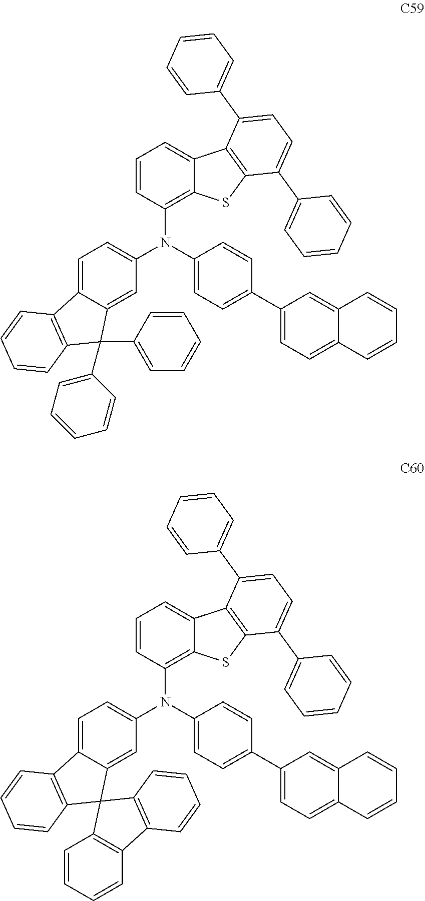 [SMILES: c1ccc(-c2ccc(-c3ccccc3)c3c2sc2c(N(c4ccc(-c5ccc6ccccc6c5)cc4)c4ccc5c(c4)C(c4ccccc4)(c4ccccc4)c4ccccc4-5)cccc23)cc1 ; c1ccc(-c2ccc(-c3ccccc3)c3c2sc2c(N(c4ccc(-c5ccc6ccccc6c5)cc4)c4ccc5c(c4)C4(c6ccccc6-c6ccccc64)c4ccccc4-5)cccc23)cc1]

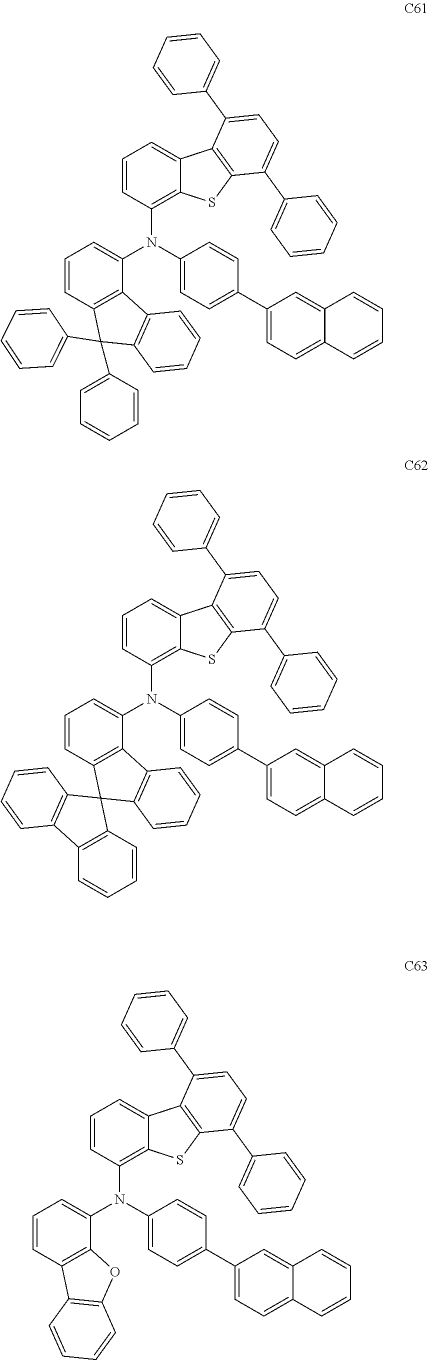 [SMILES: c1ccc(-c2ccc(-c3ccccc3)c3c2sc2c(N(c4ccc(-c5ccc6ccccc6c5)cc4)c4cccc5c4-c4ccccc4C5(c4ccccc4)c4ccccc4)cccc23)cc1 ; c1ccc(-c2ccc(-c3ccccc3)c3c2sc2c(N(c4ccc(-c5ccc6ccccc6c5)cc4)c4cccc5c4-c4ccccc4C54c5ccccc5-c5ccccc54)cccc23)cc1 ; c1ccc(-c2ccc(-c3ccccc3)c3c2sc2c(N(c4ccc(-c5ccc6ccccc6c5)cc4)c4cccc5c4oc4ccccc45)cccc23)cc1]

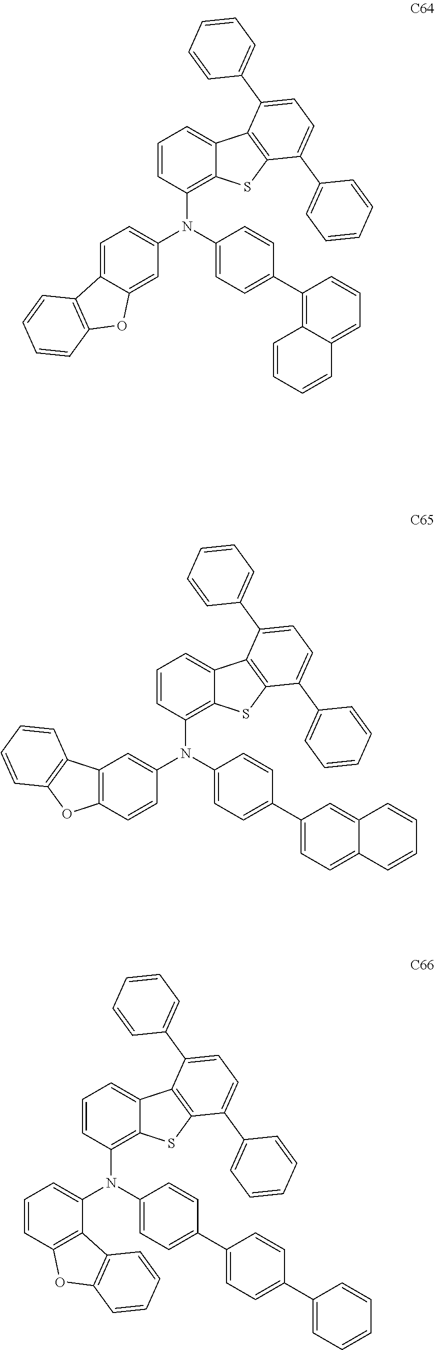 [SMILES: c1ccc(-c2ccc(-c3ccccc3)c3c2sc2c(N(c4ccc(-c5cccc6ccccc56)cc4)c4ccc5c(c4)oc4ccccc45)cccc23)cc1 ; c1ccc(-c2ccc(-c3ccccc3)c3c2sc2c(N(c4ccc(-c5ccc6ccccc6c5)cc4)c4ccc5oc6ccccc6c5c4)cccc23)cc1 ; c1ccc(-c2ccc(-c3ccc(N(c4cccc5c4sc4c(-c6ccccc6)ccc(-c6ccccc6)c45)c4cccc5oc6ccccc6c45)cc3)cc2)cc1]

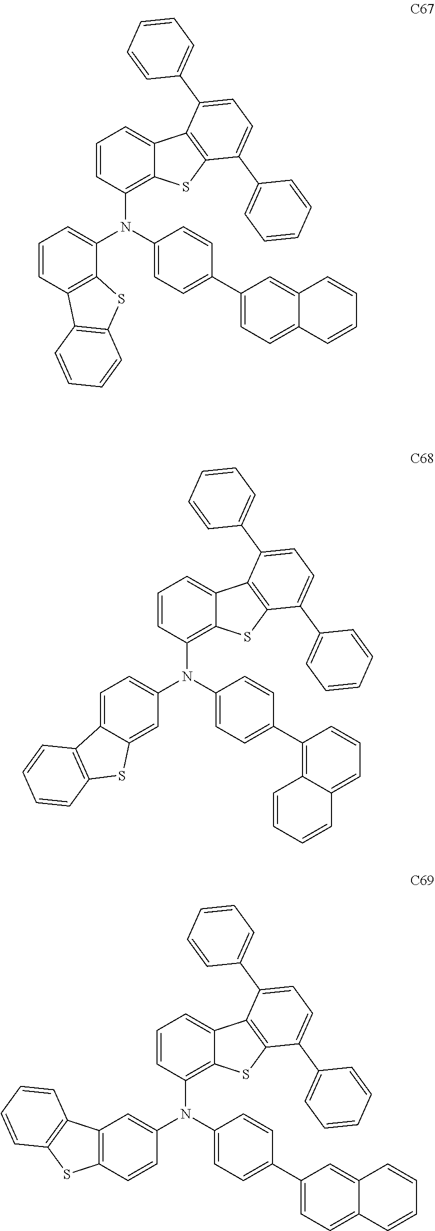 [SMILES: c1ccc(-c2ccc(-c3ccccc3)c3c2sc2c(N(c4ccc(-c5ccc6ccccc6c5)cc4)c4cccc5c4sc4ccccc45)cccc23)cc1 ; c1ccc(-c2ccc(-c3ccccc3)c3c2sc2c(N(c4ccc(-c5cccc6ccccc56)cc4)c4ccc5c(c4)sc4ccccc45)cccc23)cc1 ; c1ccc(-c2ccc(-c3ccccc3)c3c2sc2c(N(c4ccc(-c5ccc6ccccc6c5)cc4)c4ccc5sc6ccccc6c5c4)cccc23)cc1]

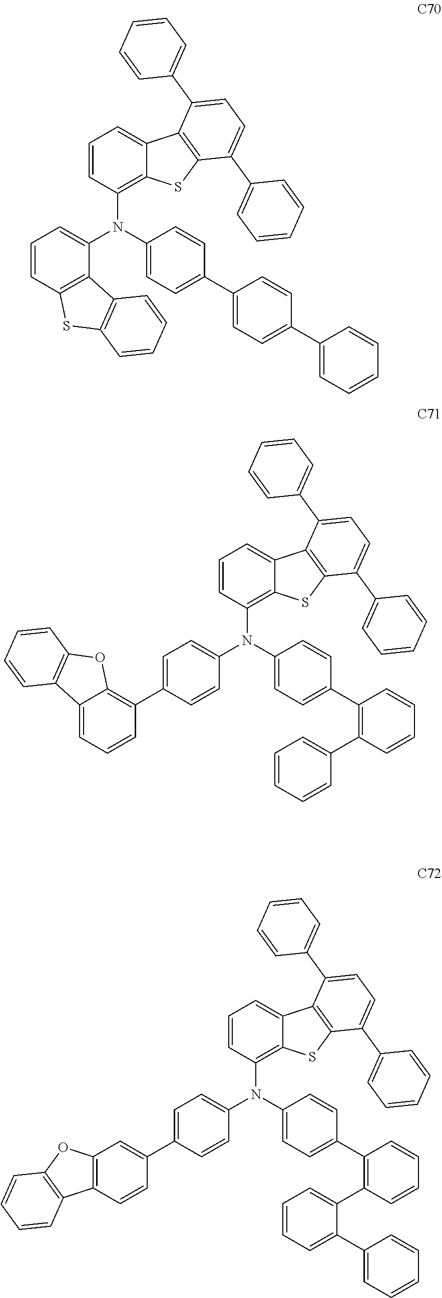 [SMILES: c1ccc(-c2ccc(-c3ccc(N(c4cccc5c4sc4c(-c6ccccc6)ccc(-c6ccccc6)c45)c4cccc5sc6ccccc6c45)cc3)cc2)cc1 ; c1ccc(-c2ccccc2-c2ccc(N(c3ccc(-c4cccc5c4oc4ccccc45)cc3)c3cccc4c3sc3c(-c5ccccc5)ccc(-c5ccccc5)c34)cc2)cc1 ; c1ccc(-c2ccccc2-c2ccccc2-c2ccc(N(c3ccc(-c4ccc5c(c4)oc4ccccc45)cc3)c3cccc4c3sc3c(-c5ccccc5)ccc(-c5ccccc5)c34)cc2)cc1]

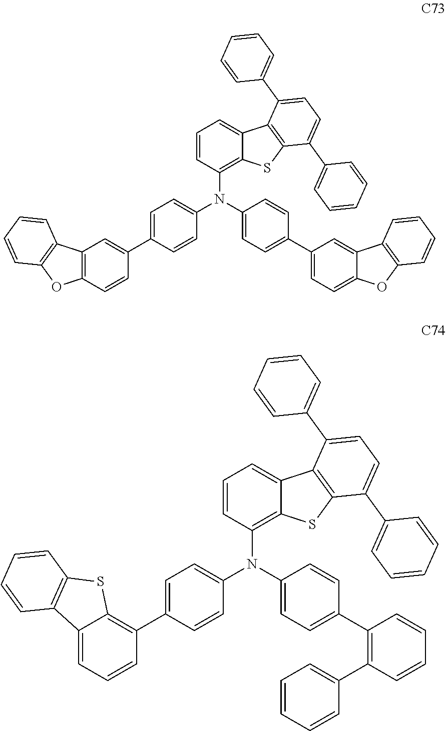 [SMILES: c1ccc(-c2ccc(-c3ccccc3)c3c2sc2c(N(c4ccc(-c5ccc6oc7ccccc7c6c5)cc4)c4ccc(-c5ccc6oc7ccccc7c6c5)cc4)cccc23)cc1 ; c1ccc(-c2ccccc2-c2ccc(N(c3ccc(-c4cccc5c4sc4ccccc45)cc3)c3cccc4c3sc3c(-c5ccccc5)ccc(-c5ccccc5)c34)cc2)cc1]

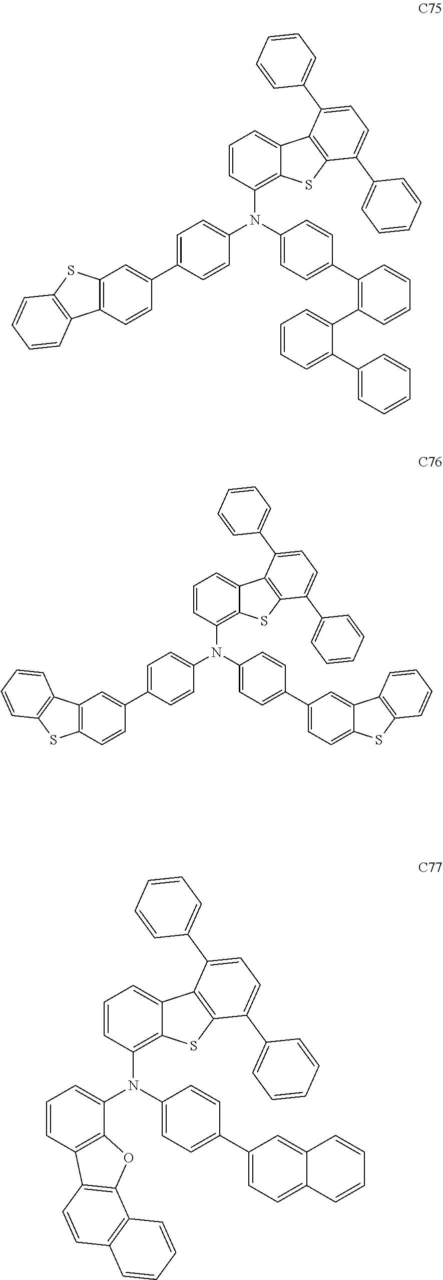 [SMILES: c1ccc(-c2ccccc2-c2ccccc2-c2ccc(N(c3ccc(-c4ccc5c(c4)sc4ccccc45)cc3)c3cccc4c3sc3c(-c5ccccc5)ccc(-c5ccccc5)c34)cc2)cc1 ; c1ccc(-c2ccc(-c3ccccc3)c3c2sc2c(N(c4ccc(-c5ccc6sc7ccccc7c6c5)cc4)c4ccc(-c5ccc6sc7ccccc7c6c5)cc4)cccc23)cc1 ; c1ccc(-c2ccc(-c3ccccc3)c3c2sc2c(N(c4ccc(-c5ccc6ccccc6c5)cc4)c4cccc5c4oc4c6ccccc6ccc54)cccc23)cc1]

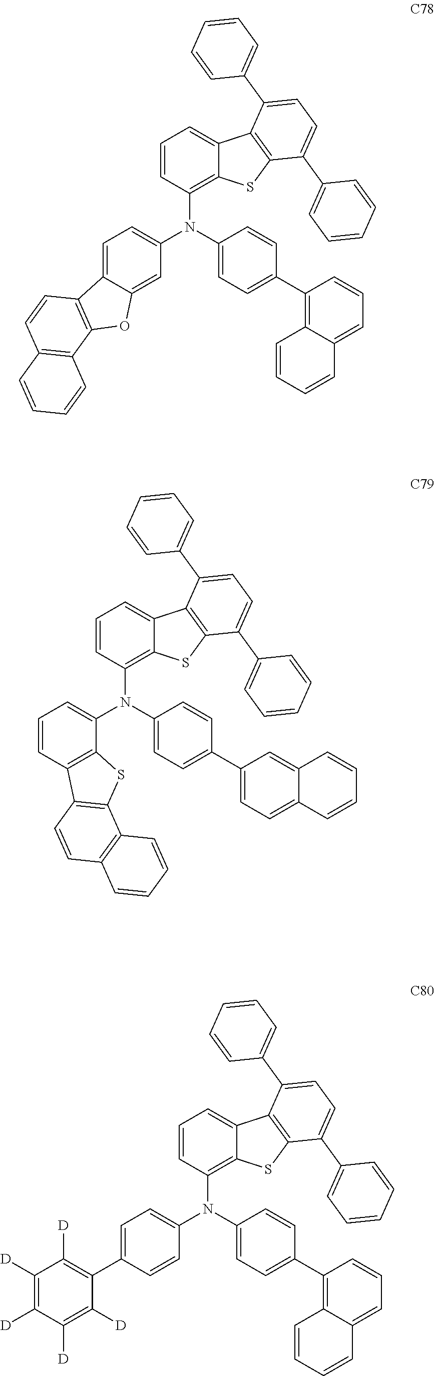 [SMILES: c1ccc(-c2ccc(-c3ccccc3)c3c2sc2c(N(c4ccc(-c5cccc6ccccc56)cc4)c4ccc5c(c4)oc4c6ccccc6ccc54)cccc23)cc1 ; c1ccc(-c2ccc(-c3ccccc3)c3c2sc2c(N(c4ccc(-c5ccc6ccccc6c5)cc4)c4cccc5c4sc4c6ccccc6ccc54)cccc23)cc1 ; [2H]c1c([2H])c([2H])c(-c2ccc(N(c3ccc(-c4cccc5ccccc45)cc3)c3cccc4c3sc3c(-c5ccccc5)ccc(-c5ccccc5)c34)cc2)c([2H])c1[2H]]

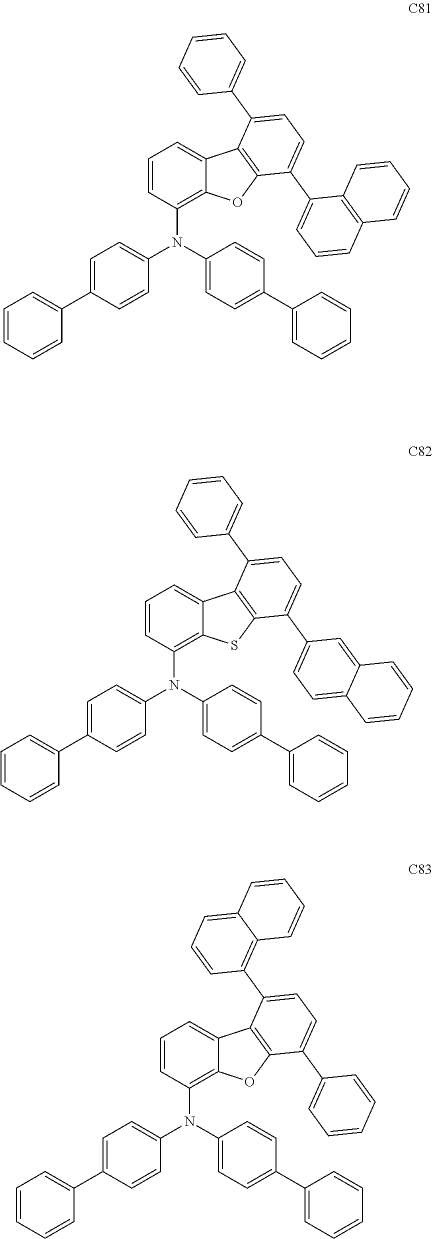 [SMILES: c1ccc(-c2ccc(N(c3ccc(-c4ccccc4)cc3)c3cccc4c3oc3c(-c5cccc6ccccc56)ccc(-c5ccccc5)c34)cc2)cc1 ; c1ccc(-c2ccc(N(c3ccc(-c4ccccc4)cc3)c3cccc4c3sc3c(-c5ccc6ccccc6c5)ccc(-c5ccccc5)c34)cc2)cc1 ; c1ccc(-c2ccc(N(c3ccc(-c4ccccc4)cc3)c3cccc4c3oc3c(-c5ccccc5)ccc(-c5cccc6ccccc56)c34)cc2)cc1]

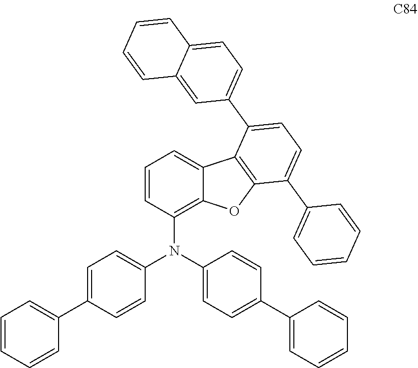 [SMILES: c1ccc(-c2ccc(N(c3ccc(-c4ccccc4)cc3)c3cccc4c3oc3c(-c5ccccc5)ccc(-c5ccc6ccccc6c5)c34)cc2)cc1]

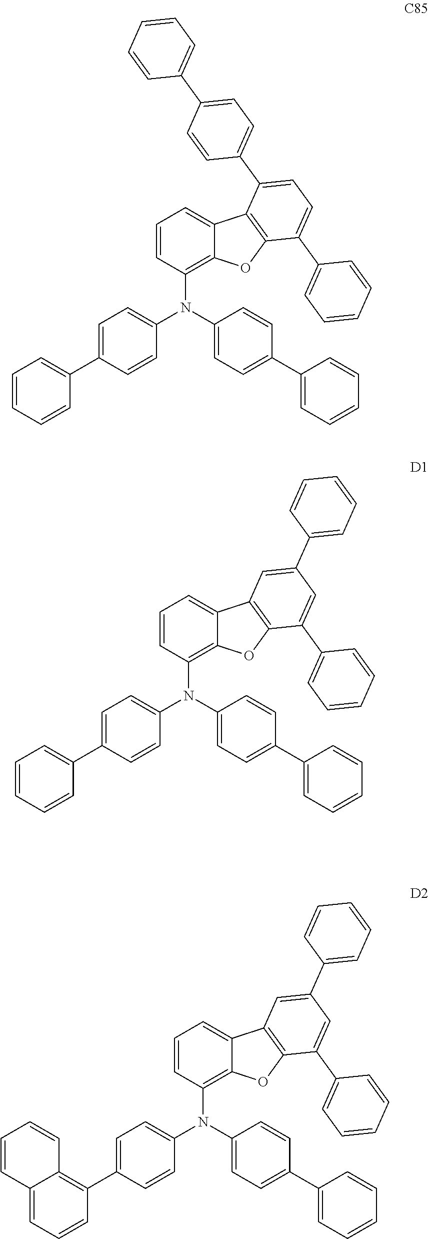 [SMILES: c1ccc(-c2ccc(-c3ccc(-c4ccccc4)c4oc5c(N(c6ccc(-c7ccccc7)cc6)c6ccc(-c7ccccc7)cc6)cccc5c34)cc2)cc1 ; c1ccc(-c2ccc(N(c3ccc(-c4ccccc4)cc3)c3cccc4c3oc3c(-c5ccccc5)cc(-c5ccccc5)cc34)cc2)cc1 ; c1ccc(-c2ccc(N(c3ccc(-c4cccc5ccccc45)cc3)c3cccc4c3oc3c(-c5ccccc5)cc(-c5ccccc5)cc34)cc2)cc1]

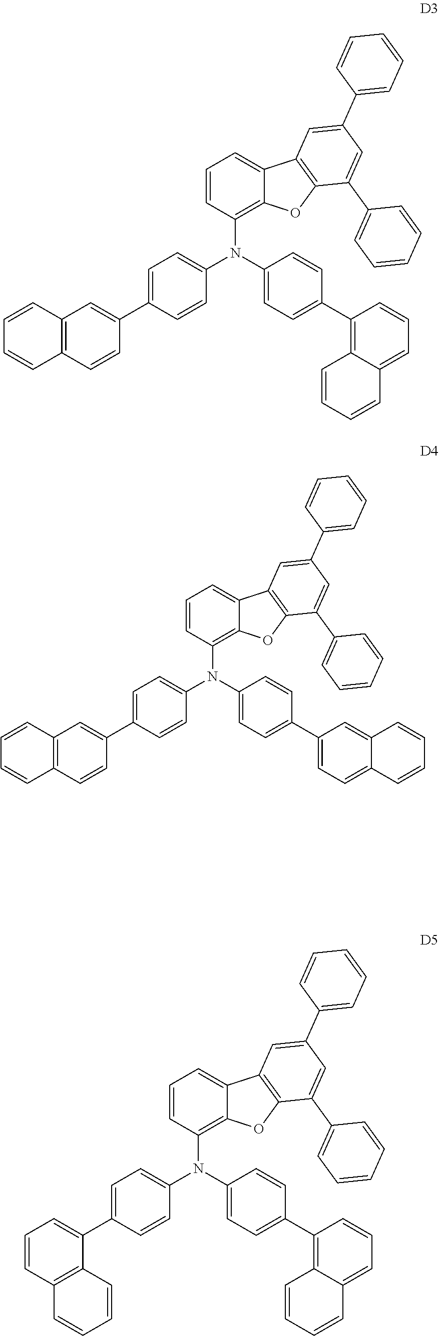 [SMILES: c1ccc(-c2cc(-c3ccccc3)c3oc4c(N(c5ccc(-c6ccc7ccccc7c6)cc5)c5ccc(-c6cccc7ccccc67)cc5)cccc4c3c2)cc1 ; c1ccc(-c2cc(-c3ccccc3)c3oc4c(N(c5ccc(-c6ccc7ccccc7c6)cc5)c5ccc(-c6ccc7ccccc7c6)cc5)cccc4c3c2)cc1 ; c1ccc(-c2cc(-c3ccccc3)c3oc4c(N(c5ccc(-c6cccc7ccccc67)cc5)c5ccc(-c6cccc7ccccc67)cc5)cccc4c3c2)cc1]

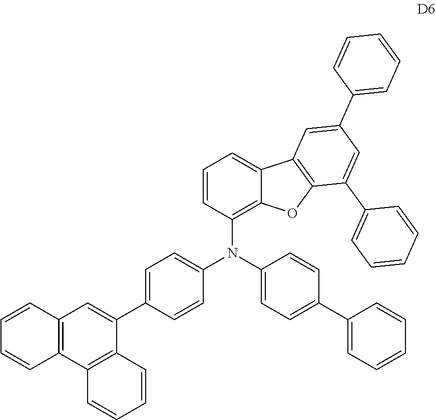 [SMILES: c1ccc(-c2ccc(N(c3ccc(-c4cc5ccccc5c5ccccc45)cc3)c3cccc4c3oc3c(-c5ccccc5)cc(-c5ccccc5)cc34)cc2)cc1]

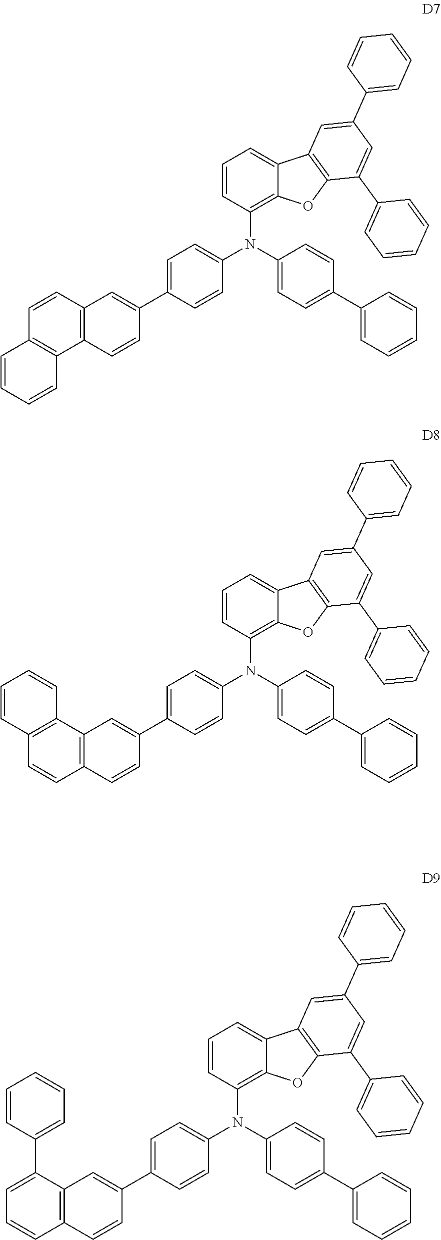 [SMILES: c1ccc(-c2ccc(N(c3ccc(-c4ccc5c(ccc6ccccc65)c4)cc3)c3cccc4c3oc3c(-c5ccccc5)cc(-c5ccccc5)cc34)cc2)cc1 ; c1ccc(-c2ccc(N(c3ccc(-c4ccc5ccc6ccccc6c5c4)cc3)c3cccc4c3oc3c(-c5ccccc5)cc(-c5ccccc5)cc34)cc2)cc1 ; c1ccc(-c2ccc(N(c3ccc(-c4ccc5cccc(-c6ccccc6)c5c4)cc3)c3cccc4c3oc3c(-c5ccccc5)cc(-c5ccccc5)cc34)cc2)cc1]

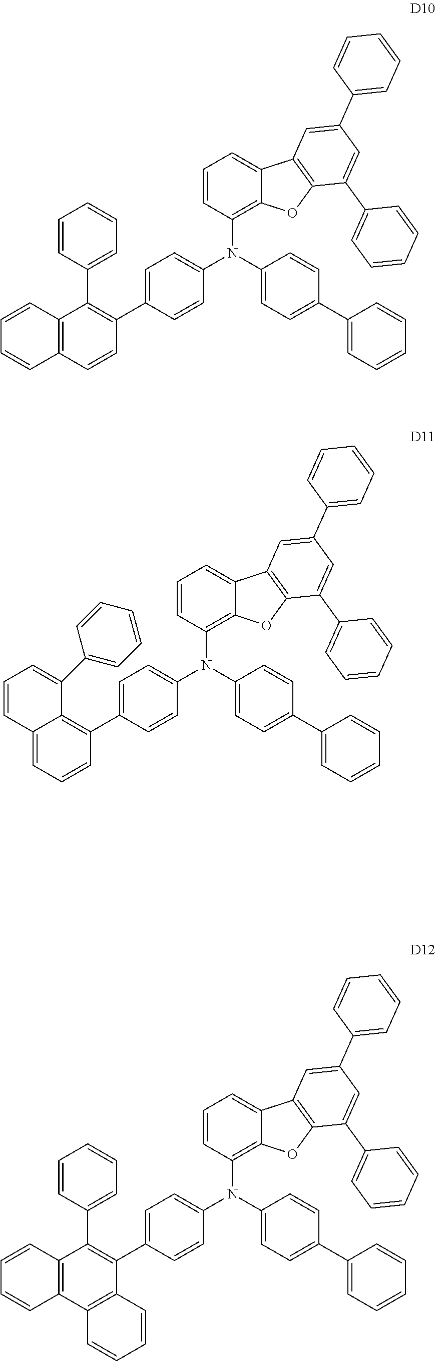 [SMILES: c1ccc(-c2ccc(N(c3ccc(-c4ccc5ccccc5c4-c4ccccc4)cc3)c3cccc4c3oc3c(-c5ccccc5)cc(-c5ccccc5)cc34)cc2)cc1 ; c1ccc(-c2ccc(N(c3ccc(-c4cccc5cccc(-c6ccccc6)c45)cc3)c3cccc4c3oc3c(-c5ccccc5)cc(-c5ccccc5)cc34)cc2)cc1 ; c1ccc(-c2ccc(N(c3ccc(-c4c(-c5ccccc5)c5ccccc5c5ccccc45)cc3)c3cccc4c3oc3c(-c5ccccc5)cc(-c5ccccc5)cc34)cc2)cc1]

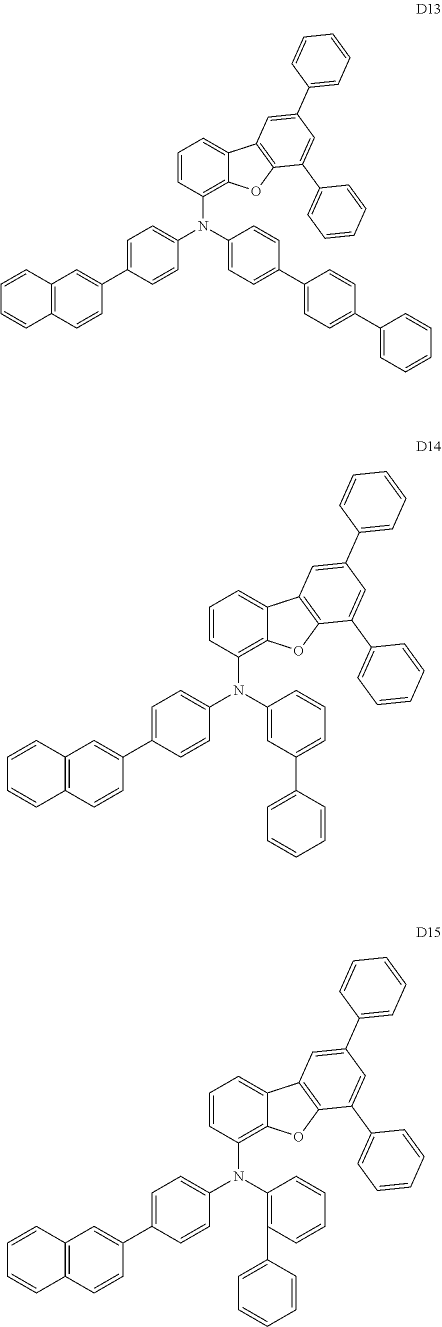 [SMILES: c1ccc(-c2ccc(-c3ccc(N(c4ccc(-c5ccc6ccccc6c5)cc4)c4cccc5c4oc4c(-c6ccccc6)cc(-c6ccccc6)cc45)cc3)cc2)cc1 ; c1ccc(-c2cccc(N(c3ccc(-c4ccc5ccccc5c4)cc3)c3cccc4c3oc3c(-c5ccccc5)cc(-c5ccccc5)cc34)c2)cc1 ; c1ccc(-c2cc(-c3ccccc3)c3oc4c(N(c5ccc(-c6ccc7ccccc7c6)cc5)c5ccccc5-c5ccccc5)cccc4c3c2)cc1]

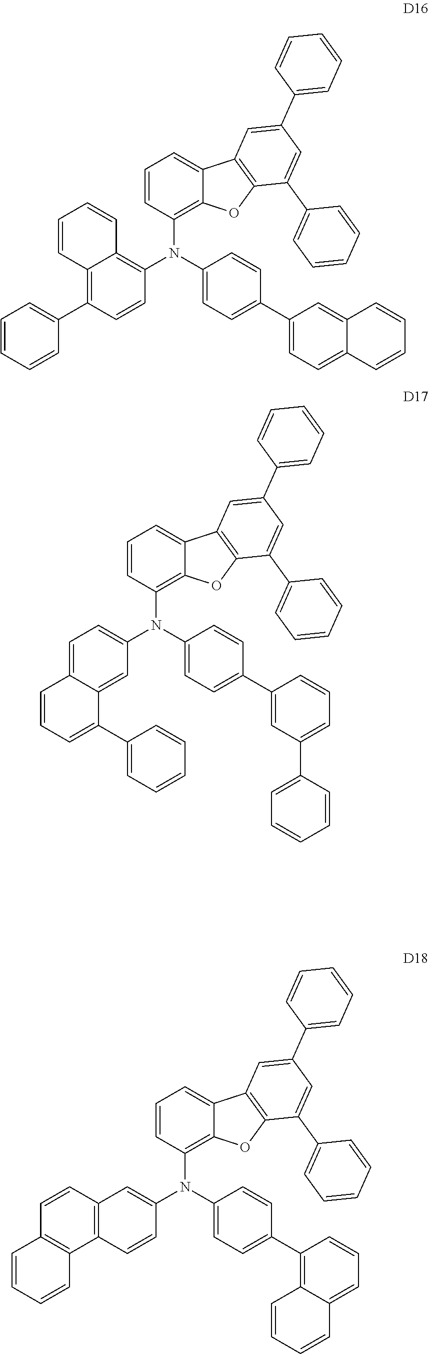

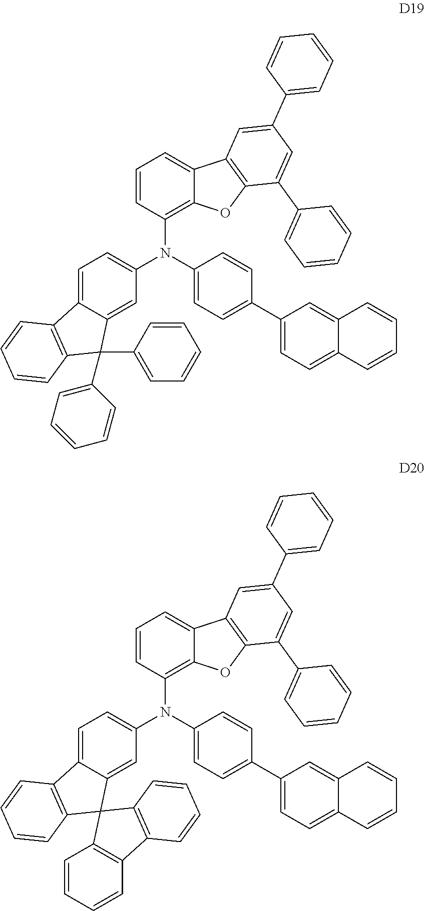 [SMILES: c1ccc(-c2cc(-c3ccccc3)c3oc4c(N(c5ccc(-c6ccc7ccccc7c6)cc5)c5ccc6c(c5)C(c5ccccc5)(c5ccccc5)c5ccccc5-6)cccc4c3c2)cc1 ; c1ccc(-c2cc(-c3ccccc3)c3oc4c(N(c5ccc(-c6ccc7ccccc7c6)cc5)c5ccc6c(c5)C5(c7ccccc7-c7ccccc75)c5ccccc5-6)cccc4c3c2)cc1]

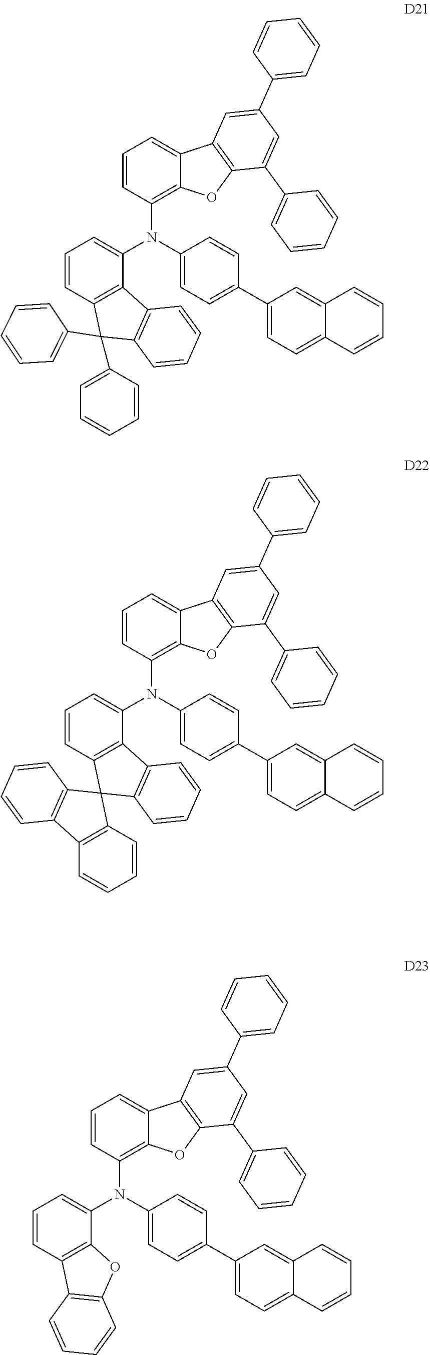 [SMILES: c1ccc(-c2cc(-c3ccccc3)c3oc4c(N(c5ccc(-c6ccc7ccccc7c6)cc5)c5cccc6c5-c5ccccc5C6(c5ccccc5)c5ccccc5)cccc4c3c2)cc1 ; c1ccc(-c2cc(-c3ccccc3)c3oc4c(N(c5ccc(-c6ccc7ccccc7c6)cc5)c5cccc6c5-c5ccccc5C65c6ccccc6-c6ccccc65)cccc4c3c2)cc1 ; c1ccc(-c2cc(-c3ccccc3)c3oc4c(N(c5ccc(-c6ccc7ccccc7c6)cc5)c5cccc6c5oc5ccccc56)cccc4c3c2)cc1]

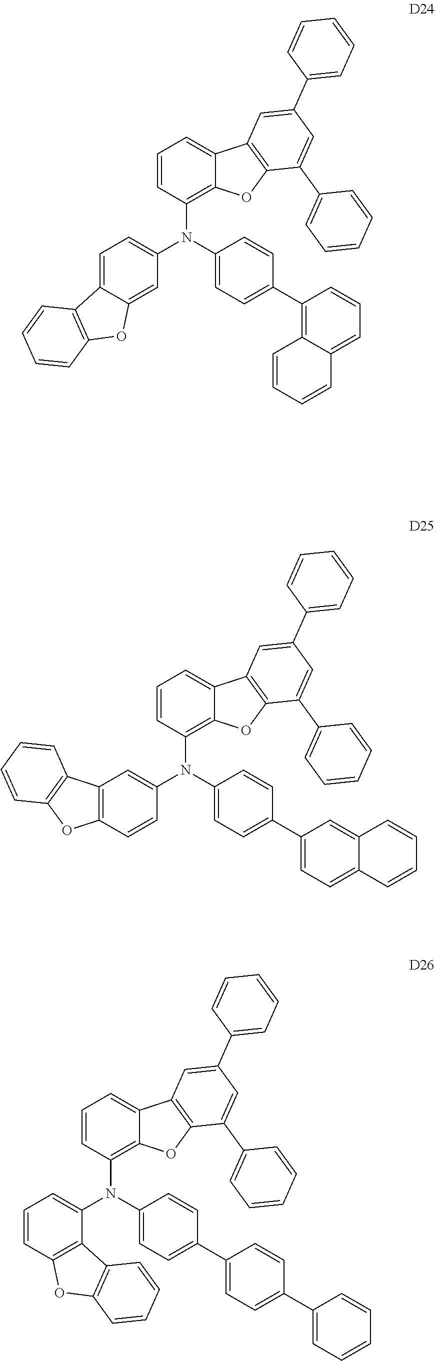 [SMILES: c1ccc(-c2cc(-c3ccccc3)c3oc4c(N(c5ccc(-c6cccc7ccccc67)cc5)c5ccc6c(c5)oc5ccccc56)cccc4c3c2)cc1 ; c1ccc(-c2cc(-c3ccccc3)c3oc4c(N(c5ccc(-c6ccc7ccccc7c6)cc5)c5ccc6oc7ccccc7c6c5)cccc4c3c2)cc1 ; c1ccc(-c2ccc(-c3ccc(N(c4cccc5c4oc4c(-c6ccccc6)cc(-c6ccccc6)cc45)c4cccc5oc6ccccc6c45)cc3)cc2)cc1]

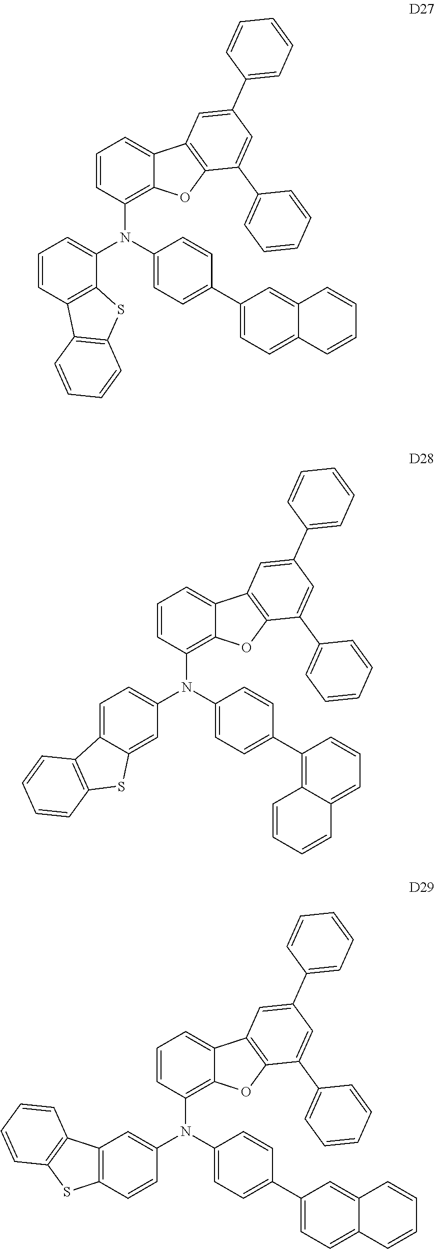 [SMILES: c1ccc(-c2cc(-c3ccccc3)c3oc4c(N(c5ccc(-c6ccc7ccccc7c6)cc5)c5cccc6c5sc5ccccc56)cccc4c3c2)cc1 ; c1ccc(-c2cc(-c3ccccc3)c3oc4c(N(c5ccc(-c6cccc7ccccc67)cc5)c5ccc6c(c5)sc5ccccc56)cccc4c3c2)cc1 ; c1ccc(-c2cc(-c3ccccc3)c3oc4c(N(c5ccc(-c6ccc7ccccc7c6)cc5)c5ccc6sc7ccccc7c6c5)cccc4c3c2)cc1]

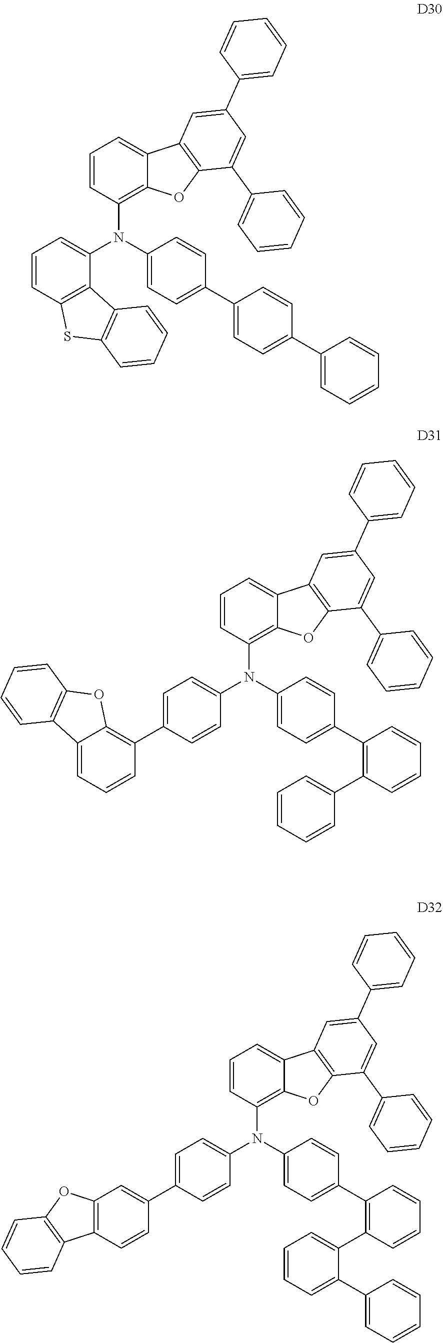 [SMILES: c1ccc(-c2ccc(-c3ccc(N(c4cccc5c4oc4c(-c6ccccc6)cc(-c6ccccc6)cc45)c4cccc5sc6ccccc6c45)cc3)cc2)cc1 ; c1ccc(-c2cc(-c3ccccc3)c3oc4c(N(c5ccc(-c6ccccc6-c6ccccc6)cc5)c5ccc(-c6cccc7c6oc6ccccc67)cc5)cccc4c3c2)cc1 ; c1ccc(-c2cc(-c3ccccc3)c3oc4c(N(c5ccc(-c6ccc7c(c6)oc6ccccc67)cc5)c5ccc(-c6ccccc6-c6ccccc6-c6ccccc6)cc5)cccc4c3c2)cc1]

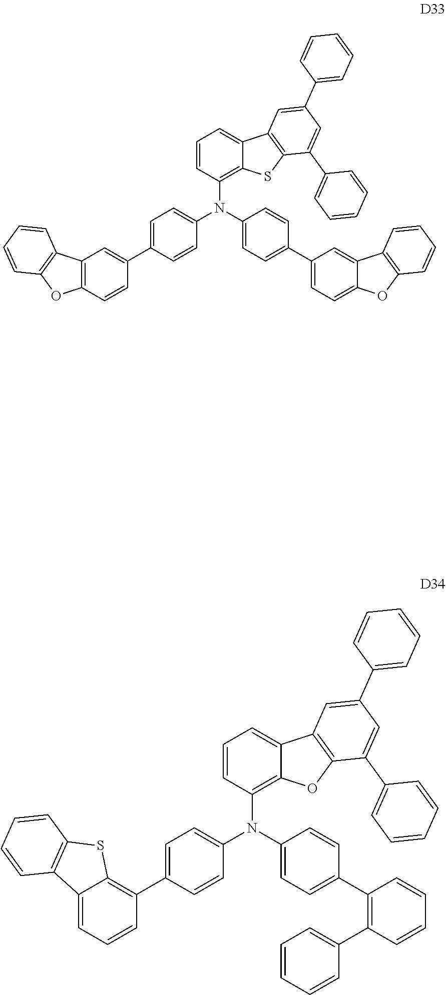 [SMILES: c1ccc(-c2cc(-c3ccccc3)c3sc4c(N(c5ccc(-c6ccc7oc8ccccc8c7c6)cc5)c5ccc(-c6ccc7oc8ccccc8c7c6)cc5)cccc4c3c2)cc1 ; c1ccc(-c2cc(-c3ccccc3)c3oc4c(N(c5ccc(-c6ccccc6-c6ccccc6)cc5)c5ccc(-c6cccc7c6sc6ccccc67)cc5)cccc4c3c2)cc1]

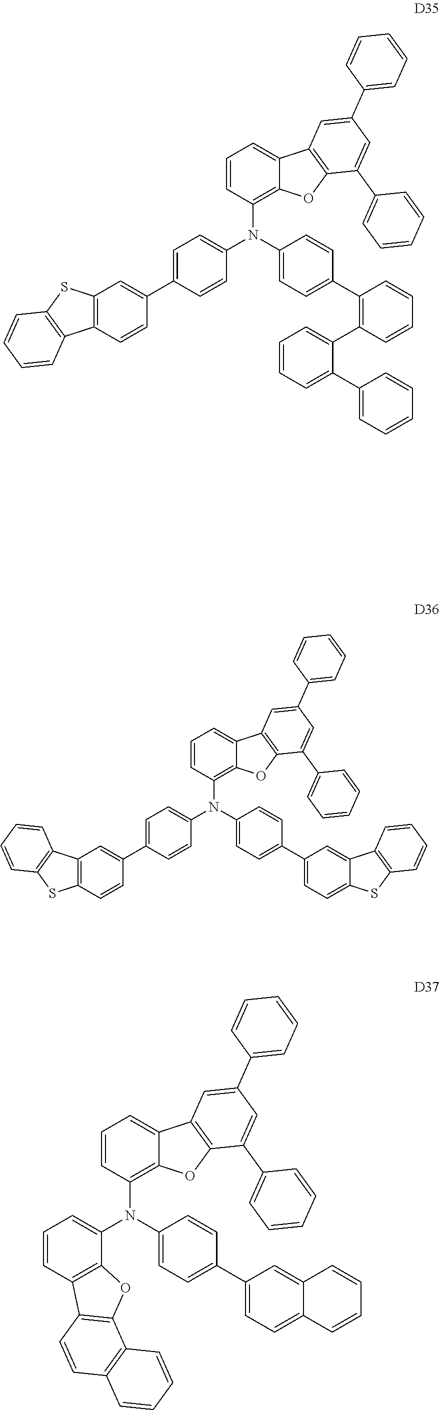 [SMILES: c1ccc(-c2cc(-c3ccccc3)c3oc4c(N(c5ccc(-c6ccc7c(c6)sc6ccccc67)cc5)c5ccc(-c6ccccc6-c6ccccc6-c6ccccc6)cc5)cccc4c3c2)cc1 ; c1ccc(-c2cc(-c3ccccc3)c3oc4c(N(c5ccc(-c6ccc7sc8ccccc8c7c6)cc5)c5ccc(-c6ccc7sc8ccccc8c7c6)cc5)cccc4c3c2)cc1 ; c1ccc(-c2cc(-c3ccccc3)c3oc4c(N(c5ccc(-c6ccc7ccccc7c6)cc5)c5cccc6c5oc5c7ccccc7ccc65)cccc4c3c2)cc1]

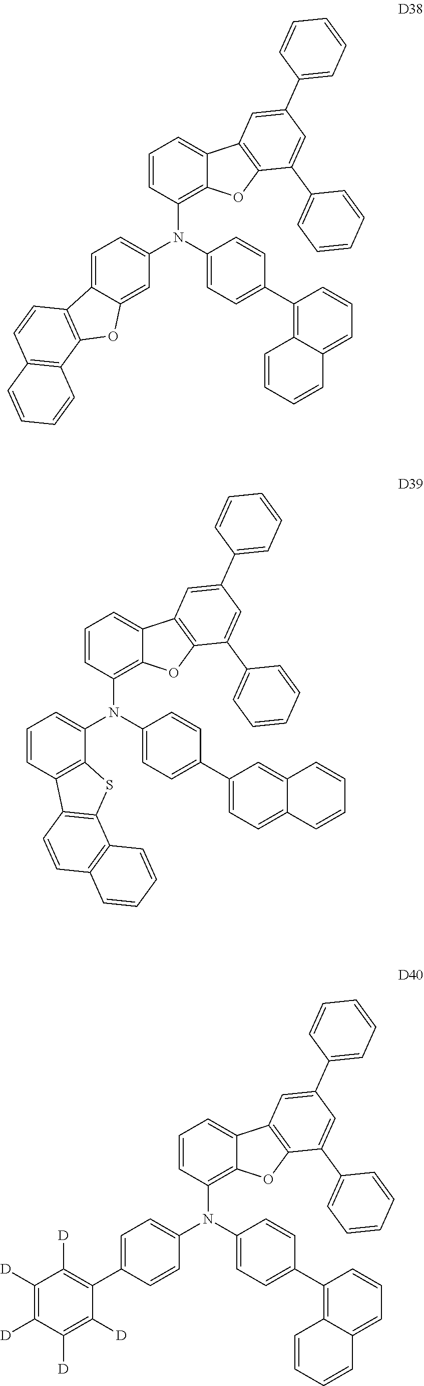 [SMILES: c1ccc(-c2cc(-c3ccccc3)c3oc4c(N(c5ccc(-c6cccc7ccccc67)cc5)c5ccc6c(c5)oc5c7ccccc7ccc65)cccc4c3c2)cc1 ; c1ccc(-c2cc(-c3ccccc3)c3oc4c(N(c5ccc(-c6ccc7ccccc7c6)cc5)c5cccc6c5sc5c7ccccc7ccc65)cccc4c3c2)cc1 ; [2H]c1c([2H])c([2H])c(-c2ccc(N(c3ccc(-c4cccc5ccccc45)cc3)c3cccc4c3oc3c(-c5ccccc5)cc(-c5ccccc5)cc34)cc2)c([2H])c1[2H]]

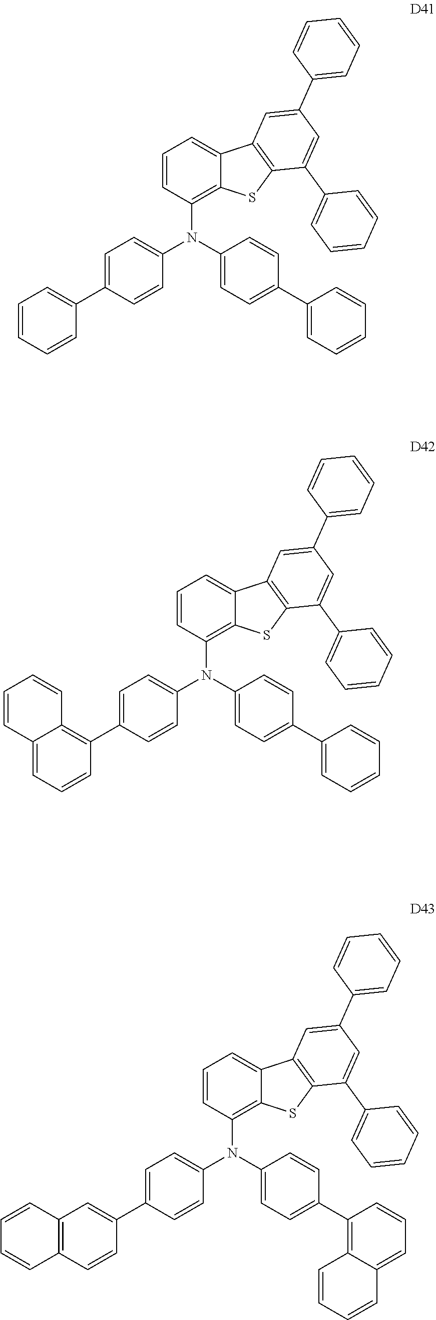 [SMILES: c1ccc(-c2ccc(N(c3ccc(-c4ccccc4)cc3)c3cccc4c3sc3c(-c5ccccc5)cc(-c5ccccc5)cc34)cc2)cc1 ; c1ccc(-c2ccc(N(c3ccc(-c4cccc5ccccc45)cc3)c3cccc4c3sc3c(-c5ccccc5)cc(-c5ccccc5)cc34)cc2)cc1 ; c1ccc(-c2cc(-c3ccccc3)c3sc4c(N(c5ccc(-c6ccc7ccccc7c6)cc5)c5ccc(-c6cccc7ccccc67)cc5)cccc4c3c2)cc1]

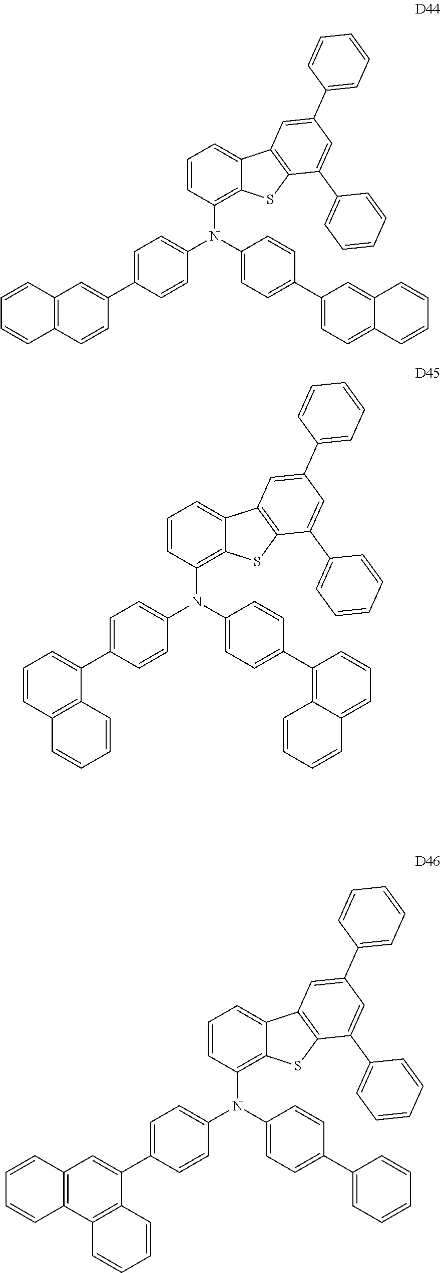

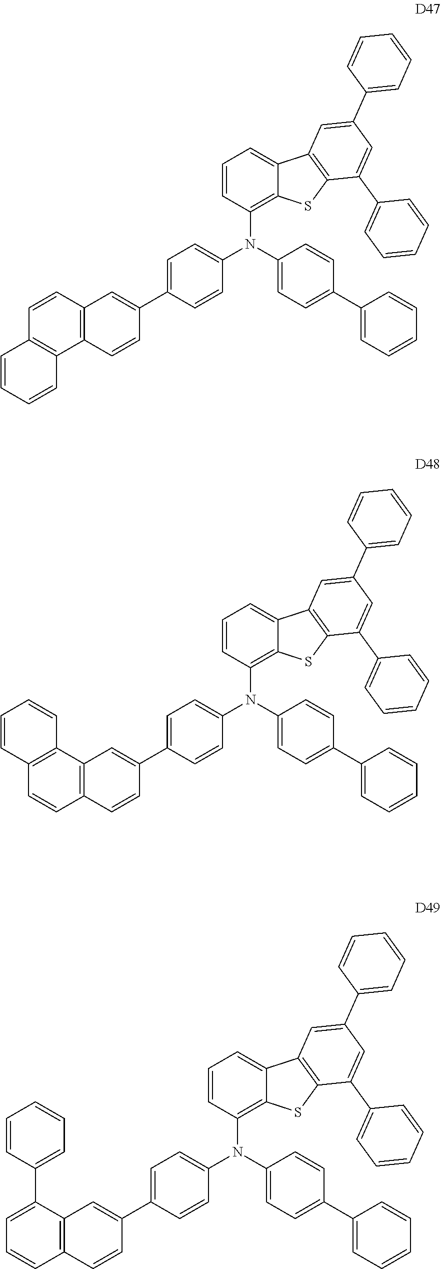 [SMILES: c1ccc(-c2ccc(N(c3ccc(-c4ccc5c(ccc6ccccc65)c4)cc3)c3cccc4c3sc3c(-c5ccccc5)cc(-c5ccccc5)cc34)cc2)cc1 ; c1ccc(-c2ccc(N(c3ccc(-c4ccc5ccc6ccccc6c5c4)cc3)c3cccc4c3sc3c(-c5ccccc5)cc(-c5ccccc5)cc34)cc2)cc1 ; c1ccc(-c2ccc(N(c3ccc(-c4ccc5cccc(-c6ccccc6)c5c4)cc3)c3cccc4c3sc3c(-c5ccccc5)cc(-c5ccccc5)cc34)cc2)cc1]

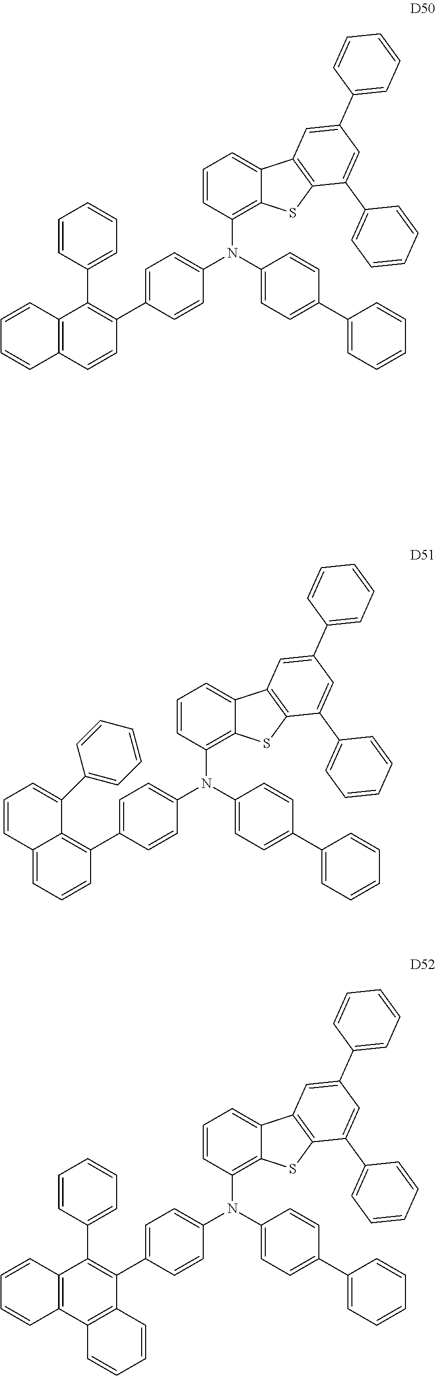 [SMILES: c1ccc(-c2ccc(N(c3ccc(-c4ccc5ccccc5c4-c4ccccc4)cc3)c3cccc4c3sc3c(-c5ccccc5)cc(-c5ccccc5)cc34)cc2)cc1 ; c1ccc(-c2ccc(N(c3ccc(-c4cccc5cccc(-c6ccccc6)c45)cc3)c3cccc4c3sc3c(-c5ccccc5)cc(-c5ccccc5)cc34)cc2)cc1 ; c1ccc(-c2ccc(N(c3ccc(-c4c(-c5ccccc5)c5ccccc5c5ccccc45)cc3)c3cccc4c3sc3c(-c5ccccc5)cc(-c5ccccc5)cc34)cc2)cc1]

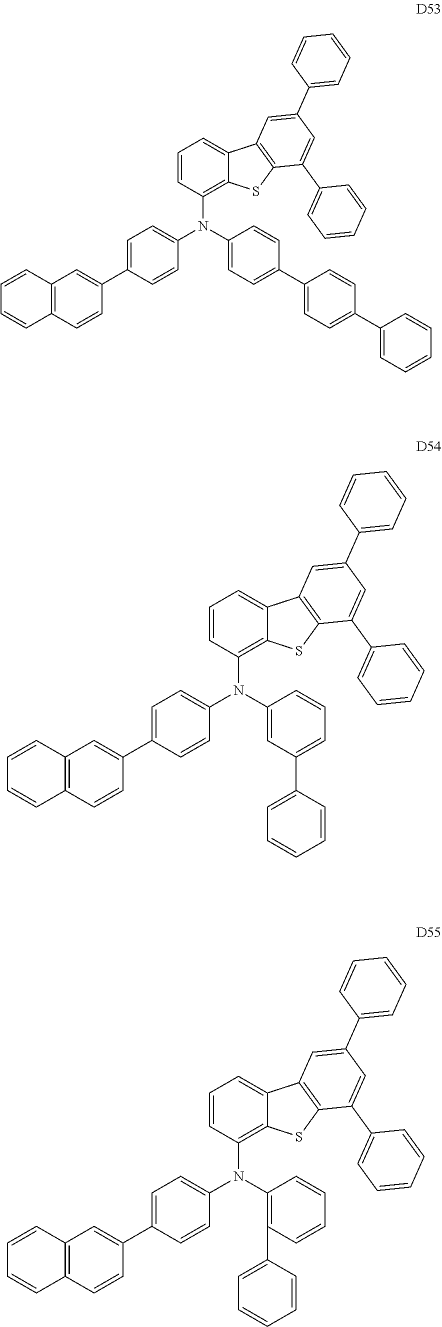 [SMILES: c1ccc(-c2ccc(-c3ccc(N(c4ccc(-c5ccc6ccccc6c5)cc4)c4cccc5c4sc4c(-c6ccccc6)cc(-c6ccccc6)cc45)cc3)cc2)cc1 ; c1ccc(-c2cccc(N(c3ccc(-c4ccc5ccccc5c4)cc3)c3cccc4c3sc3c(-c5ccccc5)cc(-c5ccccc5)cc34)c2)cc1 ; c1ccc(-c2cc(-c3ccccc3)c3sc4c(N(c5ccc(-c6ccc7ccccc7c6)cc5)c5ccccc5-c5ccccc5)cccc4c3c2)cc1]

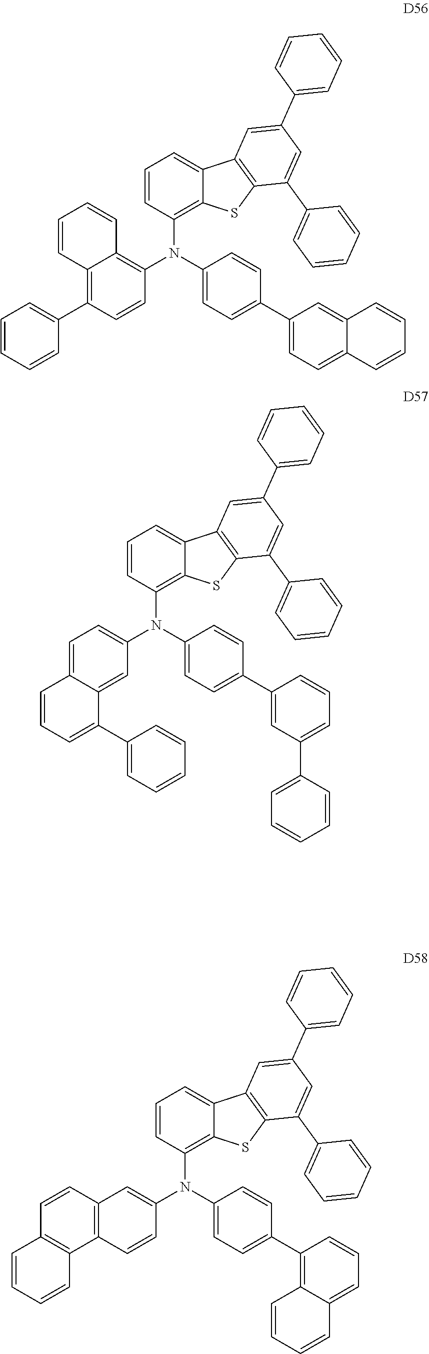

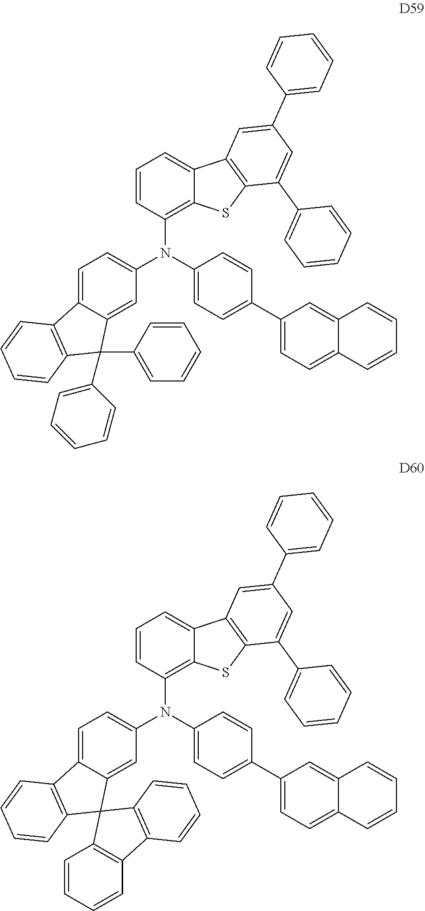 [SMILES: c1ccc(-c2cc(-c3ccccc3)c3sc4c(N(c5ccc(-c6ccc7ccccc7c6)cc5)c5ccc6c(c5)C(c5ccccc5)(c5ccccc5)c5ccccc5-6)cccc4c3c2)cc1 ; c1ccc(-c2cc(-c3ccccc3)c3sc4c(N(c5ccc(-c6ccc7ccccc7c6)cc5)c5ccc6c(c5)C5(c7ccccc7-c7ccccc75)c5ccccc5-6)cccc4c3c2)cc1]

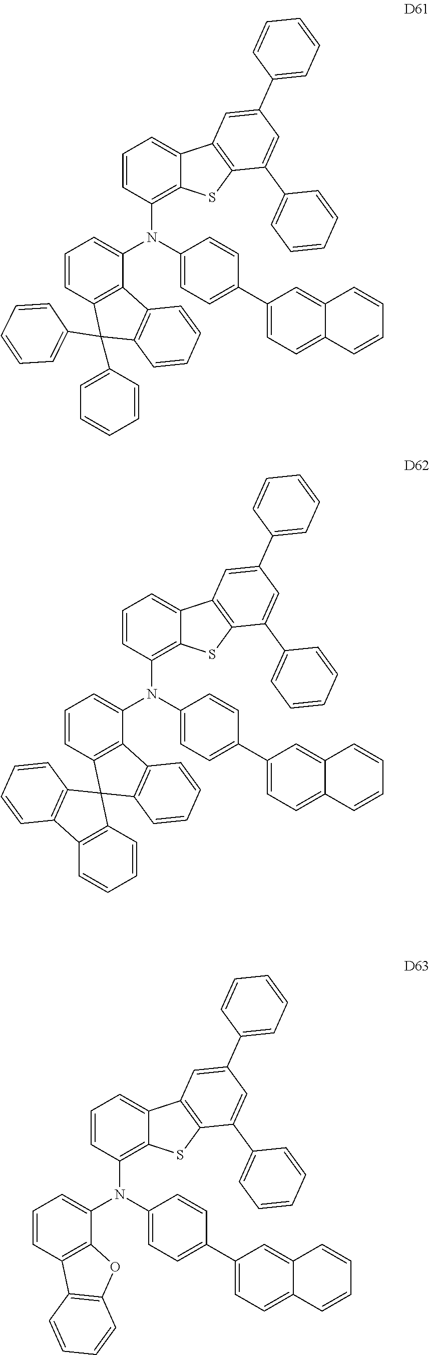 [SMILES: c1ccc(-c2cc(-c3ccccc3)c3sc4c(N(c5ccc(-c6ccc7ccccc7c6)cc5)c5cccc6c5-c5ccccc5C6(c5ccccc5)c5ccccc5)cccc4c3c2)cc1 ; c1ccc(-c2cc(-c3ccccc3)c3sc4c(N(c5ccc(-c6ccc7ccccc7c6)cc5)c5cccc6c5-c5ccccc5C65c6ccccc6-c6ccccc65)cccc4c3c2)cc1 ; c1ccc(-c2cc(-c3ccccc3)c3sc4c(N(c5ccc(-c6ccc7ccccc7c6)cc5)c5cccc6c5oc5ccccc56)cccc4c3c2)cc1]

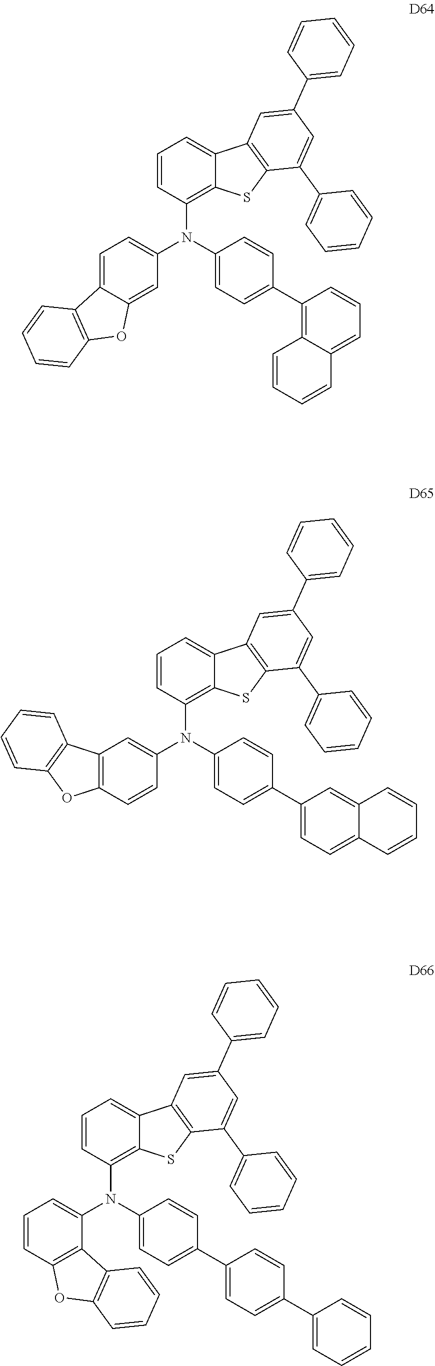 [SMILES: c1ccc(-c2cc(-c3ccccc3)c3sc4c(N(c5ccc(-c6cccc7ccccc67)cc5)c5ccc6c(c5)oc5ccccc56)cccc4c3c2)cc1 ; c1ccc(-c2cc(-c3ccccc3)c3sc4c(N(c5ccc(-c6ccc7ccccc7c6)cc5)c5ccc6oc7ccccc7c6c5)cccc4c3c2)cc1 ; c1ccc(-c2ccc(-c3ccc(N(c4cccc5c4sc4c(-c6ccccc6)cc(-c6ccccc6)cc45)c4cccc5oc6ccccc6c45)cc3)cc2)cc1]

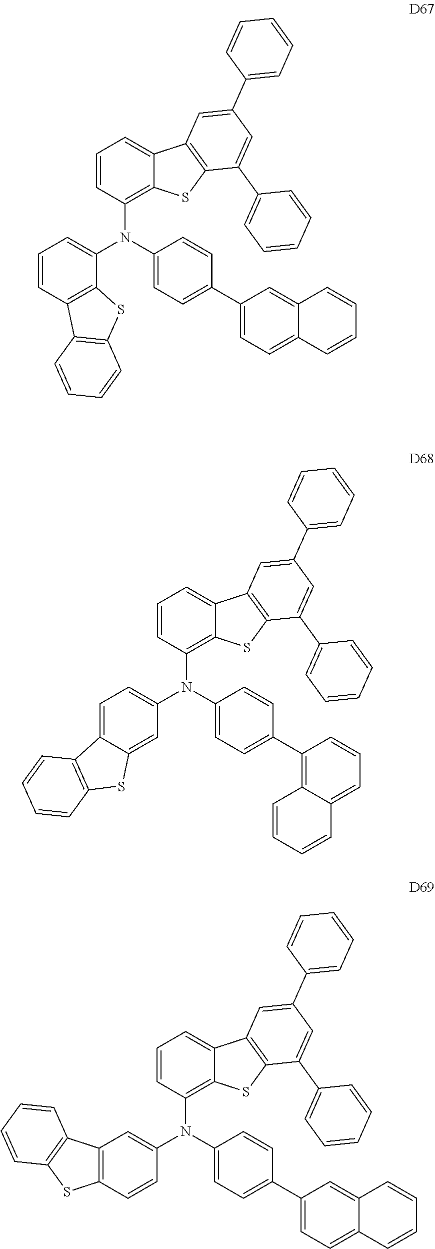 [SMILES: c1ccc(-c2cc(-c3ccccc3)c3sc4c(N(c5ccc(-c6ccc7ccccc7c6)cc5)c5cccc6c5sc5ccccc56)cccc4c3c2)cc1 ; c1ccc(-c2cc(-c3ccccc3)c3sc4c(N(c5ccc(-c6cccc7ccccc67)cc5)c5ccc6c(c5)sc5ccccc56)cccc4c3c2)cc1 ; c1ccc(-c2cc(-c3ccccc3)c3sc4c(N(c5ccc(-c6ccc7ccccc7c6)cc5)c5ccc6sc7ccccc7c6c5)cccc4c3c2)cc1]

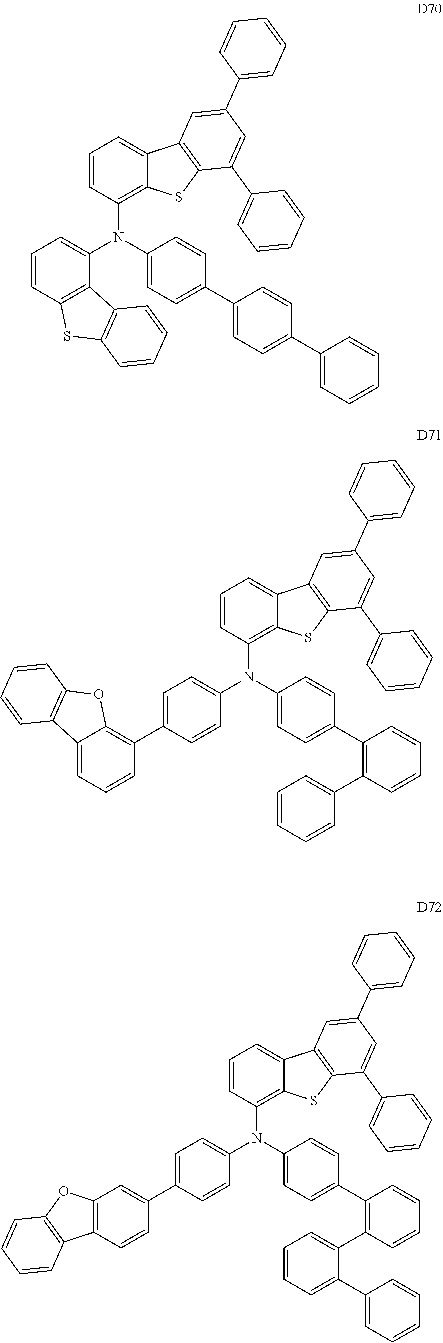 [SMILES: c1ccc(-c2ccc(-c3ccc(N(c4cccc5c4sc4c(-c6ccccc6)cc(-c6ccccc6)cc45)c4cccc5sc6ccccc6c45)cc3)cc2)cc1 ; c1ccc(-c2cc(-c3ccccc3)c3sc4c(N(c5ccc(-c6ccccc6-c6ccccc6)cc5)c5ccc(-c6cccc7c6oc6ccccc67)cc5)cccc4c3c2)cc1 ; c1ccc(-c2cc(-c3ccccc3)c3sc4c(N(c5ccc(-c6ccc7c(c6)oc6ccccc67)cc5)c5ccc(-c6ccccc6-c6ccccc6-c6ccccc6)cc5)cccc4c3c2)cc1]

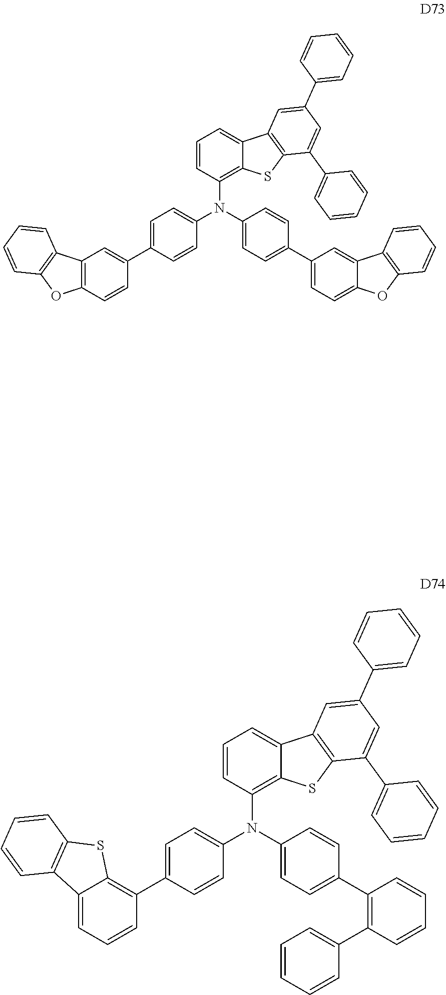 [SMILES: c1ccc(-c2cc(-c3ccccc3)c3sc4c(N(c5ccc(-c6ccc7oc8ccccc8c7c6)cc5)c5ccc(-c6ccc7oc8ccccc8c7c6)cc5)cccc4c3c2)cc1 ; c1ccc(-c2cc(-c3ccccc3)c3sc4c(N(c5ccc(-c6ccccc6-c6ccccc6)cc5)c5ccc(-c6cccc7c6sc6ccccc67)cc5)cccc4c3c2)cc1]

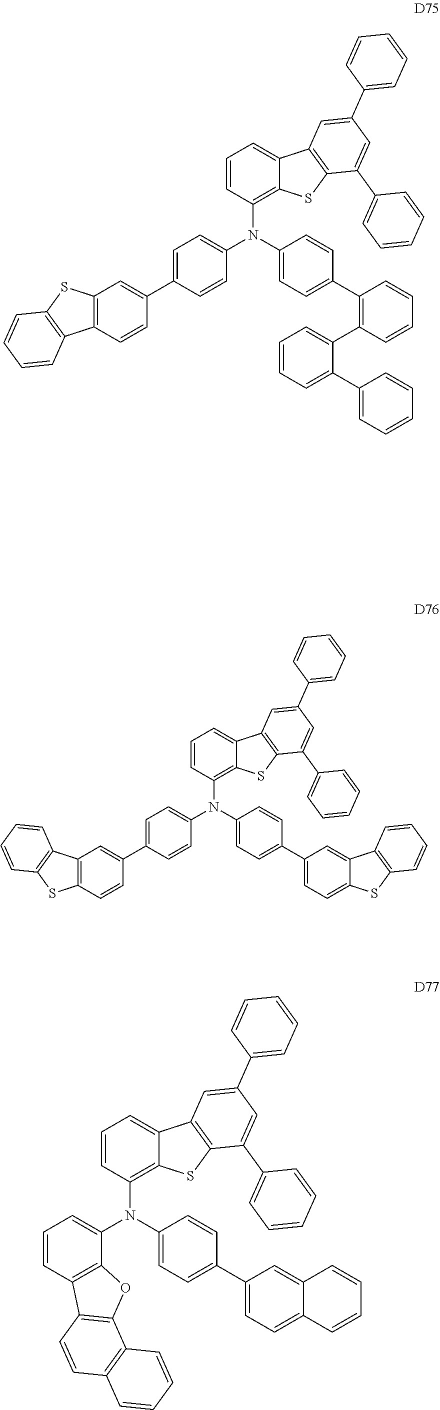 [SMILES: c1ccc(-c2cc(-c3ccccc3)c3sc4c(N(c5ccc(-c6ccc7c(c6)sc6ccccc67)cc5)c5ccc(-c6ccccc6-c6ccccc6-c6ccccc6)cc5)cccc4c3c2)cc1 ; c1ccc(-c2cc(-c3ccccc3)c3sc4c(N(c5ccc(-c6ccc7sc8ccccc8c7c6)cc5)c5ccc(-c6ccc7sc8ccccc8c7c6)cc5)cccc4c3c2)cc1 ; c1ccc(-c2cc(-c3ccccc3)c3sc4c(N(c5ccc(-c6ccc7ccccc7c6)cc5)c5cccc6c5oc5c7ccccc7ccc65)cccc4c3c2)cc1]

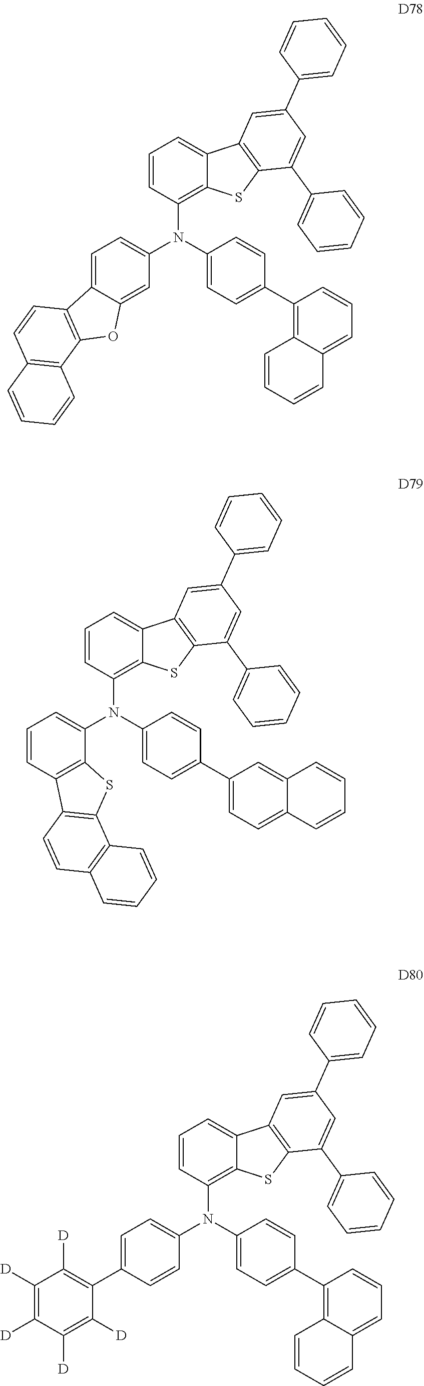 [SMILES: c1ccc(-c2cc(-c3ccccc3)c3sc4c(N(c5ccc(-c6cccc7ccccc67)cc5)c5ccc6c(c5)oc5c7ccccc7ccc65)cccc4c3c2)cc1 ; c1ccc(-c2cc(-c3ccccc3)c3sc4c(N(c5ccc(-c6ccc7ccccc7c6)cc5)c5cccc6c5sc5c7ccccc7ccc65)cccc4c3c2)cc1 ; [2H]c1c([2H])c([2H])c(-c2ccc(N(c3ccc(-c4cccc5ccccc45)cc3)c3cccc4c3sc3c(-c5ccccc5)cc(-c5ccccc5)cc34)cc2)c([2H])c1[2H]]

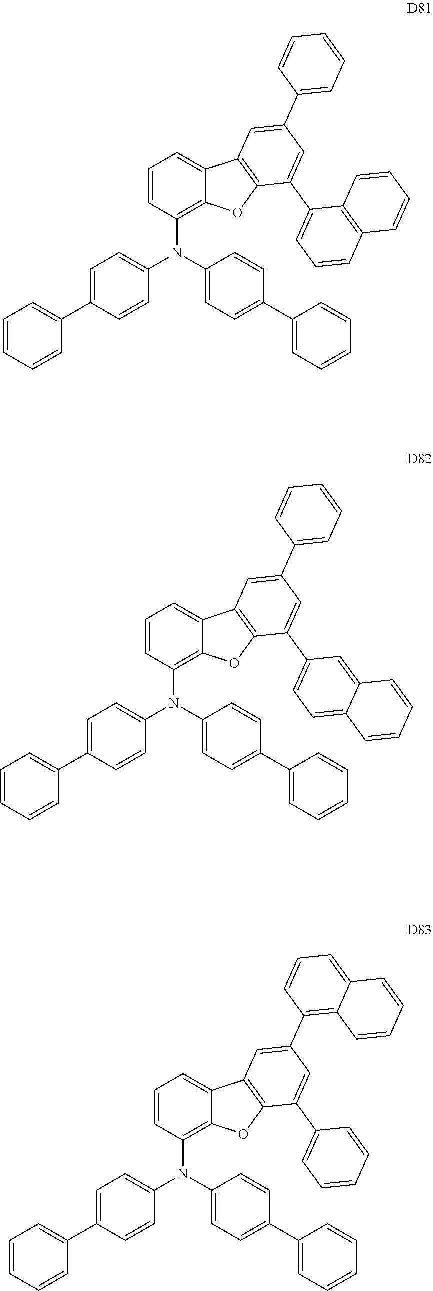 [SMILES: c1ccc(-c2ccc(N(c3ccc(-c4ccccc4)cc3)c3cccc4c3oc3c(-c5cccc6ccccc56)cc(-c5ccccc5)cc34)cc2)cc1 ; c1ccc(-c2ccc(N(c3ccc(-c4ccccc4)cc3)c3cccc4c3oc3c(-c5ccc6ccccc6c5)cc(-c5ccccc5)cc34)cc2)cc1 ; c1ccc(-c2ccc(N(c3ccc(-c4ccccc4)cc3)c3cccc4c3oc3c(-c5ccccc5)cc(-c5cccc6ccccc56)cc34)cc2)cc1]

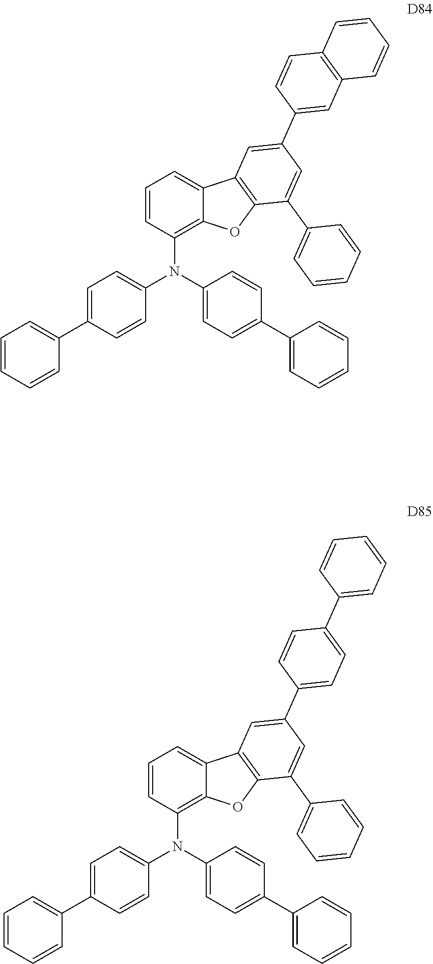 [SMILES: c1ccc(-c2ccc(N(c3ccc(-c4ccccc4)cc3)c3cccc4c3oc3c(-c5ccccc5)cc(-c5ccc6ccccc6c5)cc34)cc2)cc1 ; c1ccc(-c2ccc(-c3cc(-c4ccccc4)c4oc5c(N(c6ccc(-c7ccccc7)cc6)c6ccc(-c7ccccc7)cc6)cccc5c4c3)cc2)cc1]

14. An amine compound represented by Formula 1: 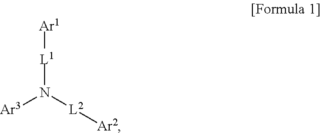
wherein in Formula 1,
L¹ and L² are each independently a direct linkage, a substituted or unsubstituted arylene group having 6 to 30 ring-forming carbon atoms, or a substituted or unsubstituted heteroarylene group having 5 to 30 ring-forming carbon atoms,
Ar¹ and Ar² are each independently a substituted or unsubstituted aryl group having 6 to 30 ring-forming carbon atoms, or a substituted or unsubstituted heteroaryl group having 5 to 30 ring-forming carbon atoms,
in case where Ar¹ or Ar² is a substituted or unsubstituted fluorenyl group, any one selected from among two benzene rings forming the 3-ring fluorenyl group is bonded with L¹ and L², or bonded with the nitrogen atom of Formula 1,
L¹-Ar¹ and L²-Ar² do not comprise a carbazole group or a silyl group, and
Ar³ is different from Ar¹ and Ar², and is represented by Formula 2: 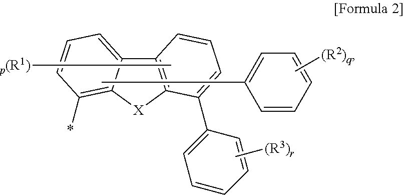
wherein in Formula 2,
X is O or S,
R¹ is a hydrogen atom, a deuterium atom, a halogen atom, or a substituted or unsubstituted alkyl group having 1 to 20 carbon atoms,
R² and R³ are each independently a hydrogen atom, a deuterium atom, a halogen atom, a substituted or unsubstituted alkyl group having 1 to 20 carbon atoms, or a substituted or unsubstituted aryl group having 6 to 30 ring-forming carbon atoms, or combined with an adjacent group to form a hydrocarbon ring,
“p” is an integer of 0 to 6, and “q” and “r” are each independently an integer of 0 to 5, and
*- is a position combined with the nitrogen atom of Formula 1.

15. The amine compound of claim 14, wherein Formula 2 is represented by any one selected from among Formula 2-1 to Formula 2-4: 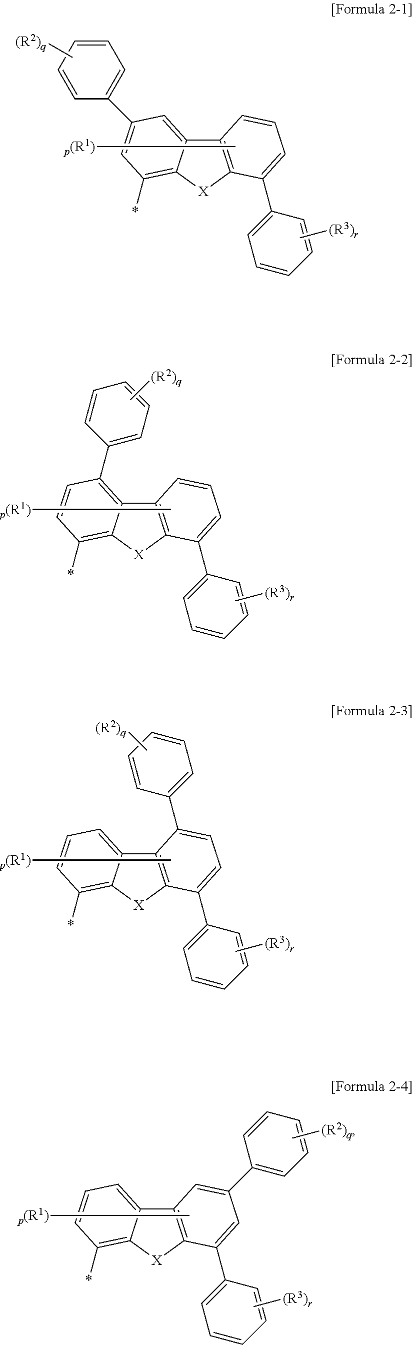
wherein in Formula 2-1 to Formula 2-4, X, R¹, R², R³, “p”, “q” and “r” are the same as respectively defined in Formula 2.

16. The amine compound of claim 14, wherein Formula 1 is represented by Formula 3: 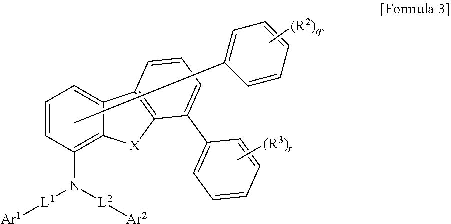
wherein in Formula 3, X, L¹, L², Ar¹, Ar², R², R³, “q” and “r” are the same as respectively defined in Formula 1 and Formula 2.

17. The amine compound of claim 16, wherein Formula 3 is represented by any one selected from among Formula 3-1 to Formula 3-4: 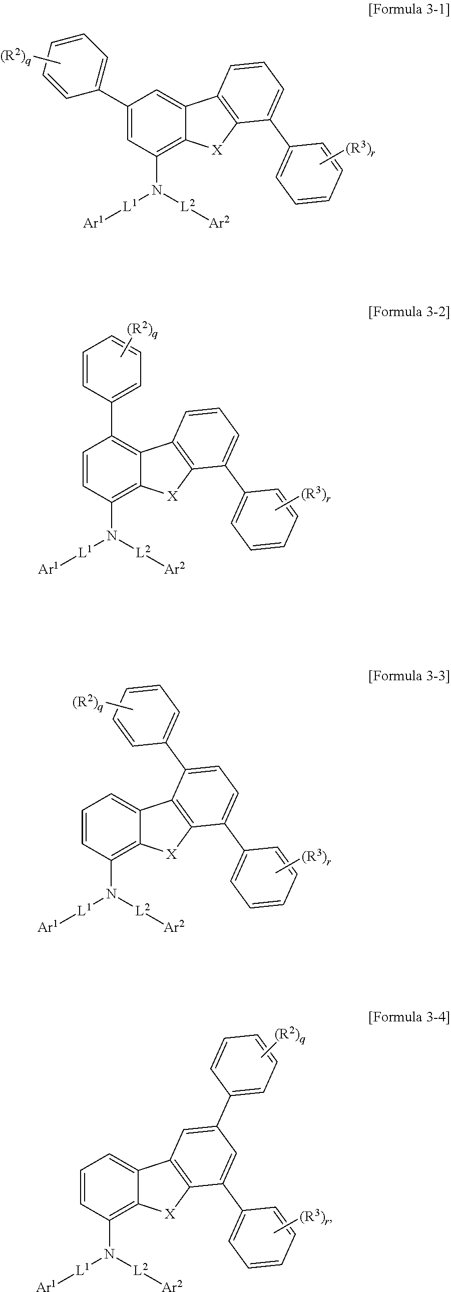
wherein in Formula 3-1 to Formula 3-4, X, L¹, L², Ar¹, Ar², R², R³, “q” and “r” are the same as respectively defined in Formula 1 and Formula 2.

18. The amine compound of claim 14, wherein R² and R³ are each independently a hydrogen atom, a deuterium atom, or a substituted or unsubstituted phenyl group, or at least one selected from among R² and R³ is combined with a phenyl group substituent to form a substituted or unsubstituted naphthyl group.

19. The amine compound of claim 14, wherein Ar¹ and Ar² are each independently a substituted or unsubstituted phenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted phenanthrenyl group, a substituted or unsubstituted fluorenyl group, a substituted or unsubstituted dibenzofuran group, a substituted or unsubstituted dibenzothiophene group, a substituted or unsubstituted benzonaphthofuran group, or a substituted or unsubstituted benzonaphthothiophene group.

20. The amine compound of claim 14, wherein the amine compound represented by Formula 1 is represented by any one selected from among compounds in Compound Group 1: 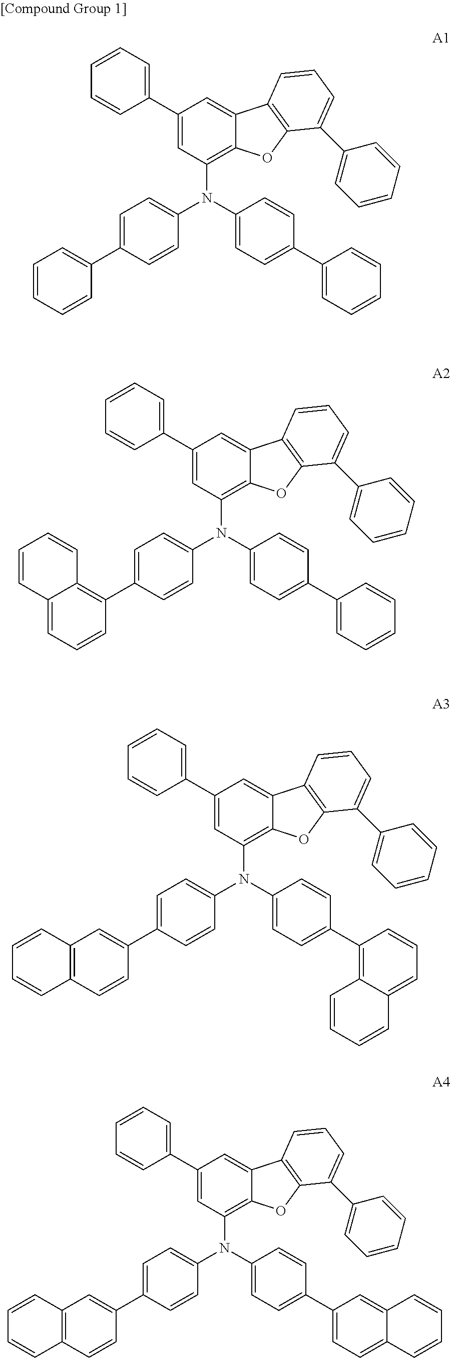 [SMILES: c1ccc(-c2ccc(N(c3ccc(-c4ccccc4)cc3)c3cc(-c4ccccc4)cc4c3oc3c(-c5ccccc5)cccc34)cc2)cc1 ; c1ccc(-c2ccc(N(c3ccc(-c4cccc5ccccc45)cc3)c3cc(-c4ccccc4)cc4c3oc3c(-c5ccccc5)cccc34)cc2)cc1 ; c1ccc(-c2cc(N(c3ccc(-c4ccc5ccccc5c4)cc3)c3ccc(-c4cccc5ccccc45)cc3)c3oc4c(-c5ccccc5)cccc4c3c2)cc1 ; c1ccc(-c2cc(N(c3ccc(-c4ccc5ccccc5c4)cc3)c3ccc(-c4ccc5ccccc5c4)cc3)c3oc4c(-c5ccccc5)cccc4c3c2)cc1] 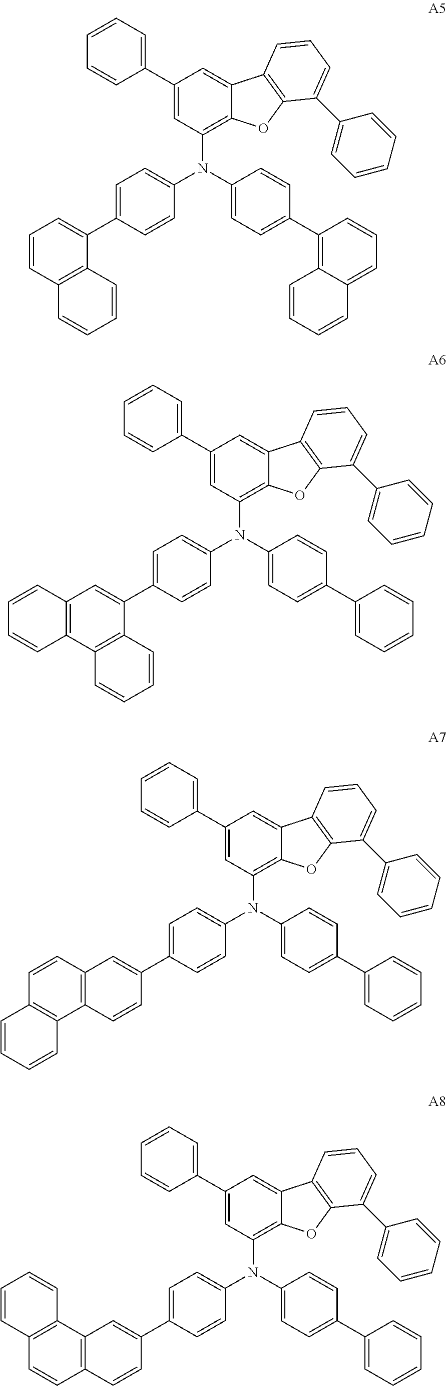 [SMILES: c1ccc(-c2cc(N(c3ccc(-c4cccc5ccccc45)cc3)c3ccc(-c4cccc5ccccc45)cc3)c3oc4c(-c5ccccc5)cccc4c3c2)cc1 ; c1ccc(-c2ccc(N(c3ccc(-c4cc5ccccc5c5ccccc45)cc3)c3cc(-c4ccccc4)cc4c3oc3c(-c5ccccc5)cccc34)cc2)cc1 ; c1ccc(-c2ccc(N(c3ccc(-c4ccc5c(ccc6ccccc65)c4)cc3)c3cc(-c4ccccc4)cc4c3oc3c(-c5ccccc5)cccc34)cc2)cc1 ; c1ccc(-c2ccc(N(c3ccc(-c4ccc5ccc6ccccc6c5c4)cc3)c3cc(-c4ccccc4)cc4c3oc3c(-c5ccccc5)cccc34)cc2)cc1] 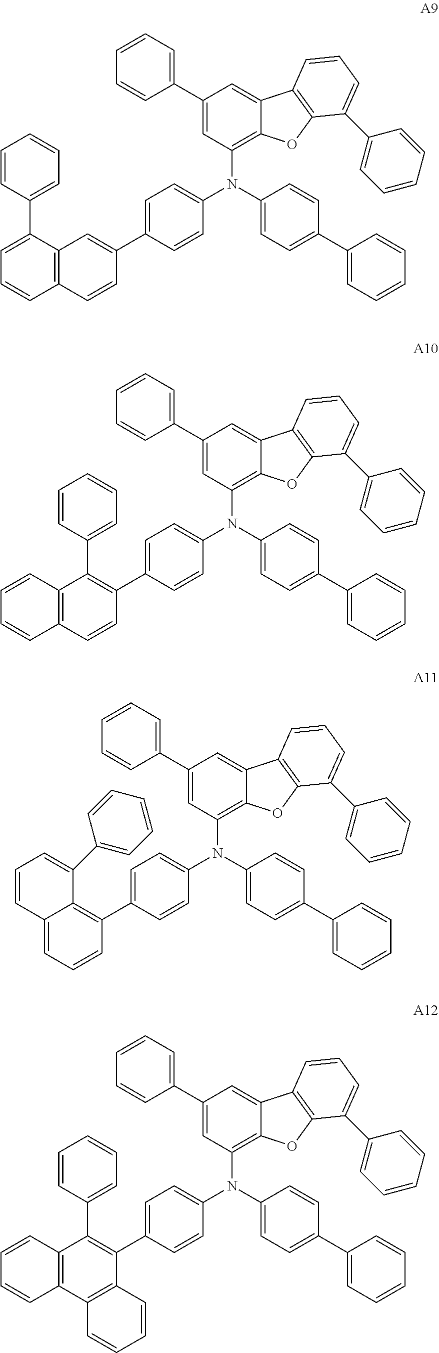 [SMILES: c1ccc(-c2ccc(N(c3ccc(-c4ccc5cccc(-c6ccccc6)c5c4)cc3)c3cc(-c4ccccc4)cc4c3oc3c(-c5ccccc5)cccc34)cc2)cc1 ; c1ccc(-c2ccc(N(c3ccc(-c4ccc5ccccc5c4-c4ccccc4)cc3)c3cc(-c4ccccc4)cc4c3oc3c(-c5ccccc5)cccc34)cc2)cc1 ; c1ccc(-c2ccc(N(c3ccc(-c4cccc5cccc(-c6ccccc6)c45)cc3)c3cc(-c4ccccc4)cc4c3oc3c(-c5ccccc5)cccc34)cc2)cc1 ; c1ccc(-c2ccc(N(c3ccc(-c4c(-c5ccccc5)c5ccccc5c5ccccc45)cc3)c3cc(-c4ccccc4)cc4c3oc3c(-c5ccccc5)cccc34)cc2)cc1] 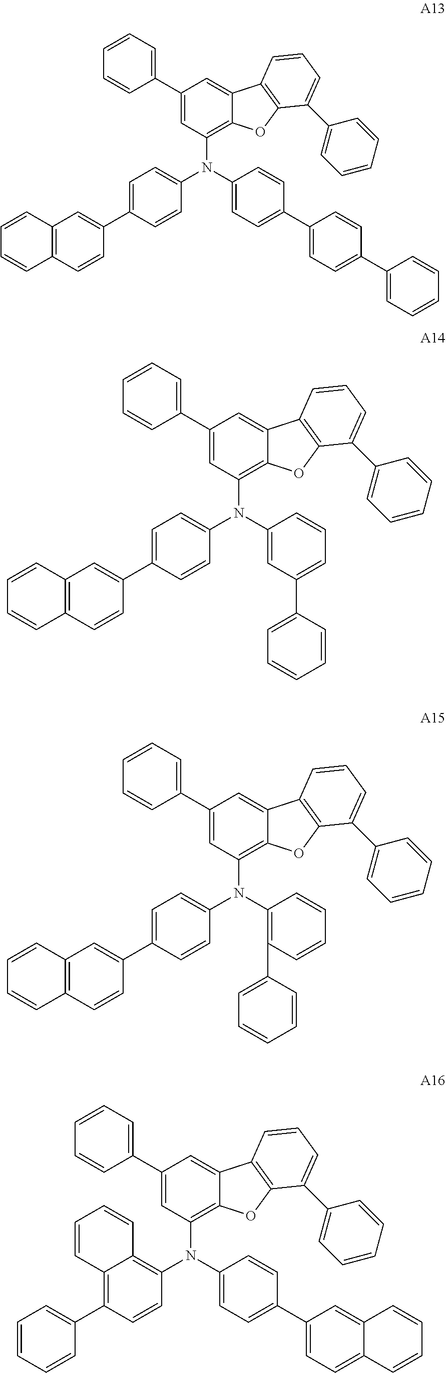 [SMILES: c1ccc(-c2ccc(-c3ccc(N(c4ccc(-c5ccc6ccccc6c5)cc4)c4cc(-c5ccccc5)cc5c4oc4c(-c6ccccc6)cccc45)cc3)cc2)cc1 ; c1ccc(-c2cccc(N(c3ccc(-c4ccc5ccccc5c4)cc3)c3cc(-c4ccccc4)cc4c3oc3c(-c5ccccc5)cccc34)c2)cc1 ; c1ccc(-c2cc(N(c3ccc(-c4ccc5ccccc5c4)cc3)c3ccccc3-c3ccccc3)c3oc4c(-c5ccccc5)cccc4c3c2)cc1 ; c1ccc(-c2cc(N(c3ccc(-c4ccc5ccccc5c4)cc3)c3ccc(-c4ccccc4)c4ccccc34)c3oc4c(-c5ccccc5)cccc4c3c2)cc1] 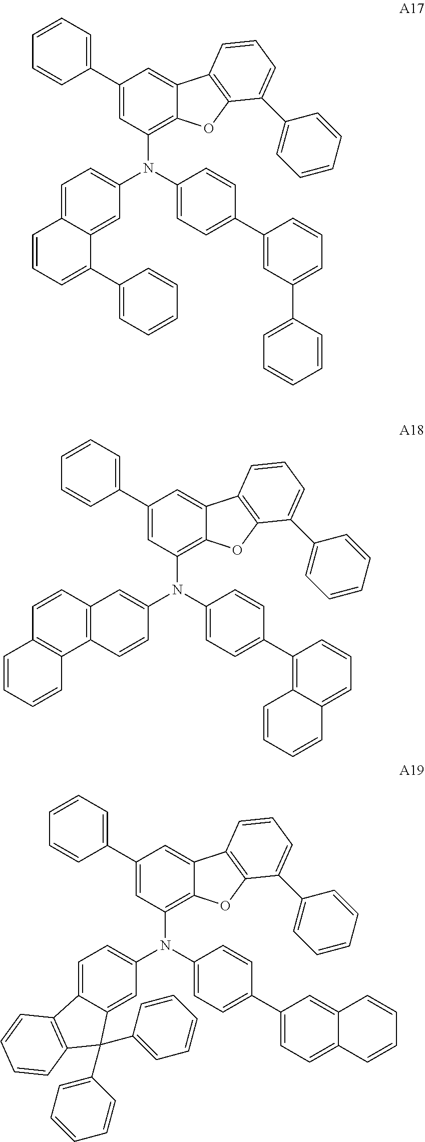 [SMILES: c1ccc(-c2cccc(-c3ccc(N(c4ccc5cccc(-c6ccccc6)c5c4)c4cc(-c5ccccc5)cc5c4oc4c(-c6ccccc6)cccc45)cc3)c2)cc1 ; c1ccc(-c2cc(N(c3ccc(-c4cccc5ccccc45)cc3)c3ccc4c(ccc5ccccc54)c3)c3oc4c(-c5ccccc5)cccc4c3c2)cc1 ; c1ccc(-c2cc(N(c3ccc(-c4ccc5ccccc5c4)cc3)c3ccc4c(c3)C(c3ccccc3)(c3ccccc3)c3ccccc3-4)c3oc4c(-c5ccccc5)cccc4c3c2)cc1] 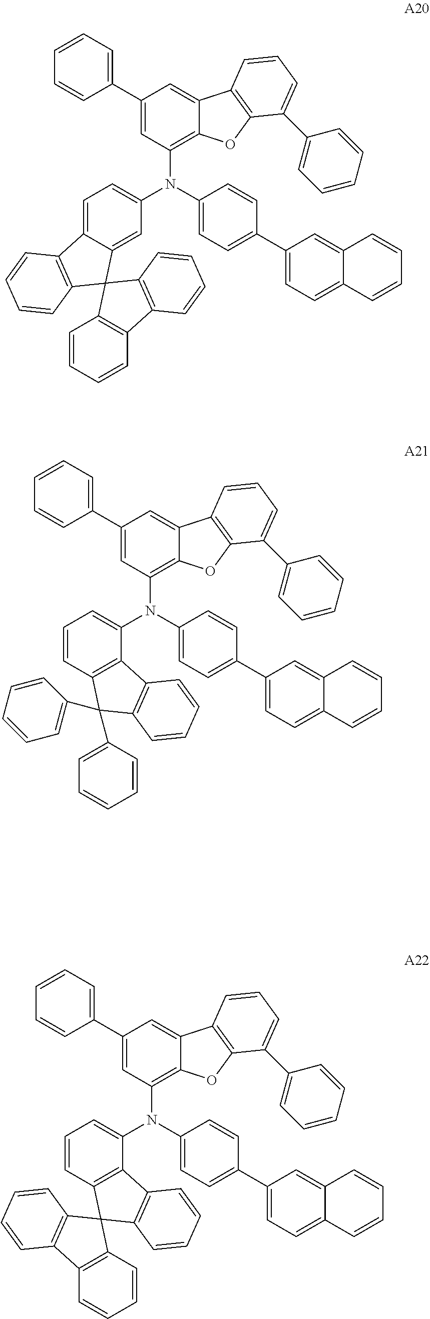 [SMILES: c1ccc(-c2cc(N(c3ccc(-c4ccc5ccccc5c4)cc3)c3ccc4c(c3)C3(c5ccccc5-c5ccccc53)c3ccccc3-4)c3oc4c(-c5ccccc5)cccc4c3c2)cc1 ; c1ccc(-c2cc(N(c3ccc(-c4ccc5ccccc5c4)cc3)c3cccc4c3-c3ccccc3C4(c3ccccc3)c3ccccc3)c3oc4c(-c5ccccc5)cccc4c3c2)cc1 ; c1ccc(-c2cc(N(c3ccc(-c4ccc5ccccc5c4)cc3)c3cccc4c3-c3ccccc3C43c4ccccc4-c4ccccc43)c3oc4c(-c5ccccc5)cccc4c3c2)cc1] 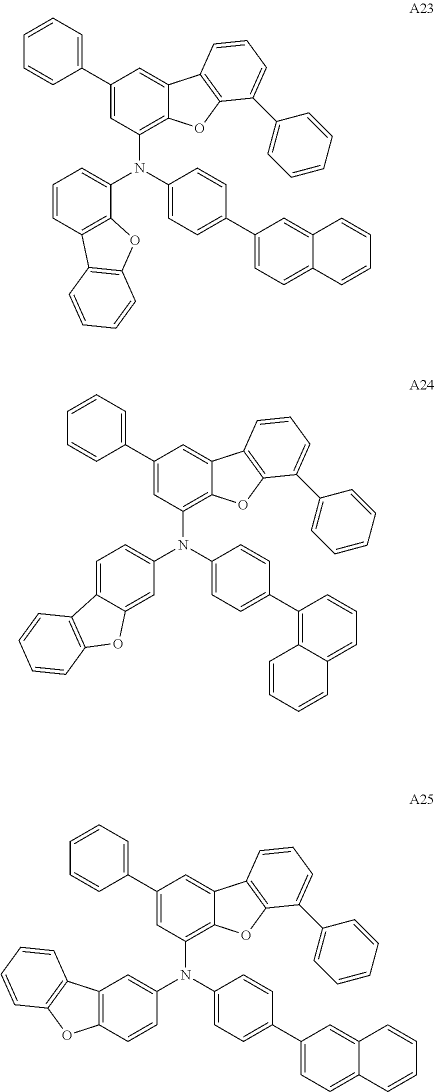 [SMILES: c1ccc(-c2cc(N(c3ccc(-c4ccc5ccccc5c4)cc3)c3cccc4c3oc3ccccc34)c3oc4c(-c5ccccc5)cccc4c3c2)cc1 ; c1ccc(-c2cc(N(c3ccc(-c4cccc5ccccc45)cc3)c3ccc4c(c3)oc3ccccc34)c3oc4c(-c5ccccc5)cccc4c3c2)cc1 ; c1ccc(-c2cc(N(c3ccc(-c4ccc5ccccc5c4)cc3)c3ccc4oc5ccccc5c4c3)c3oc4c(-c5ccccc5)cccc4c3c2)cc1] 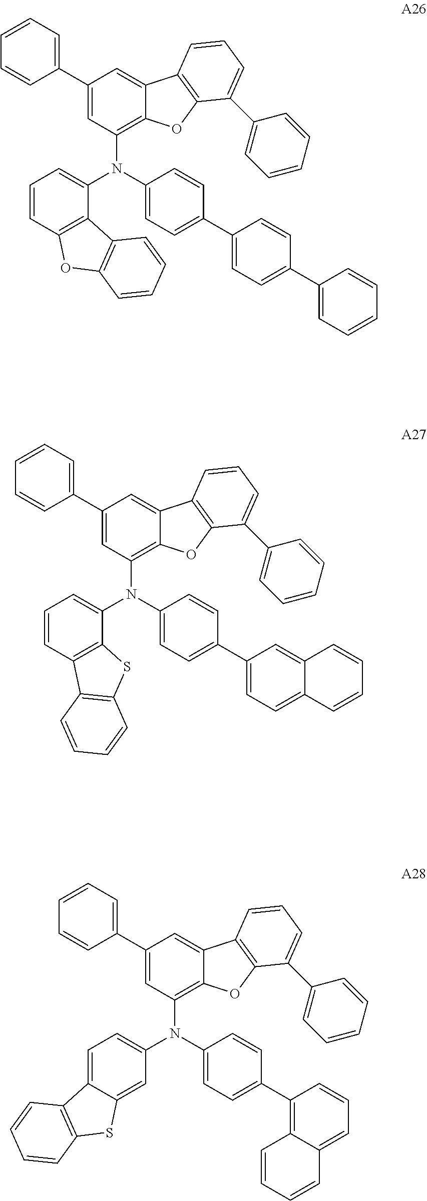 [SMILES: c1ccc(-c2ccc(-c3ccc(N(c4cc(-c5ccccc5)cc5c4oc4c(-c6ccccc6)cccc45)c4cccc5oc6ccccc6c45)cc3)cc2)cc1 ; c1ccc(-c2cc(N(c3ccc(-c4ccc5ccccc5c4)cc3)c3cccc4c3sc3ccccc34)c3oc4c(-c5ccccc5)cccc4c3c2)cc1 ; c1ccc(-c2cc(N(c3ccc(-c4cccc5ccccc45)cc3)c3ccc4c(c3)sc3ccccc34)c3oc4c(-c5ccccc5)cccc4c3c2)cc1] 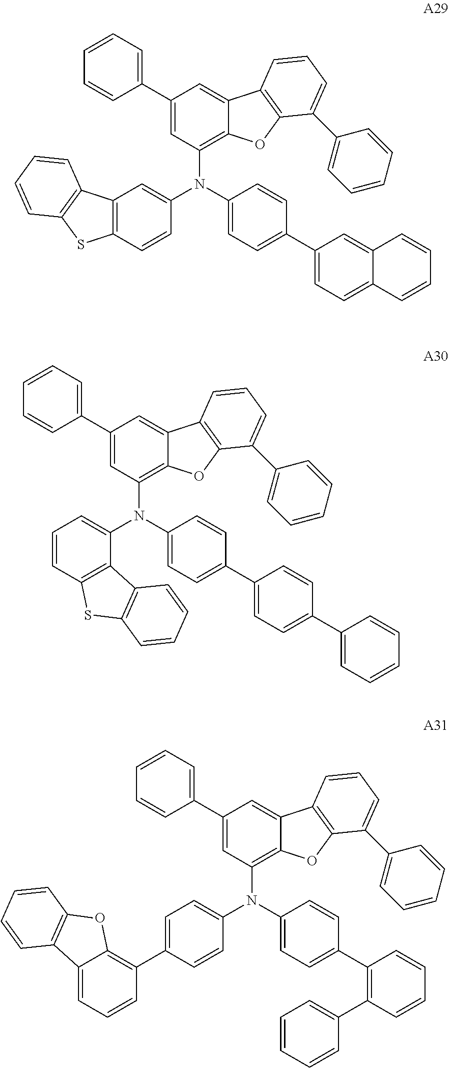 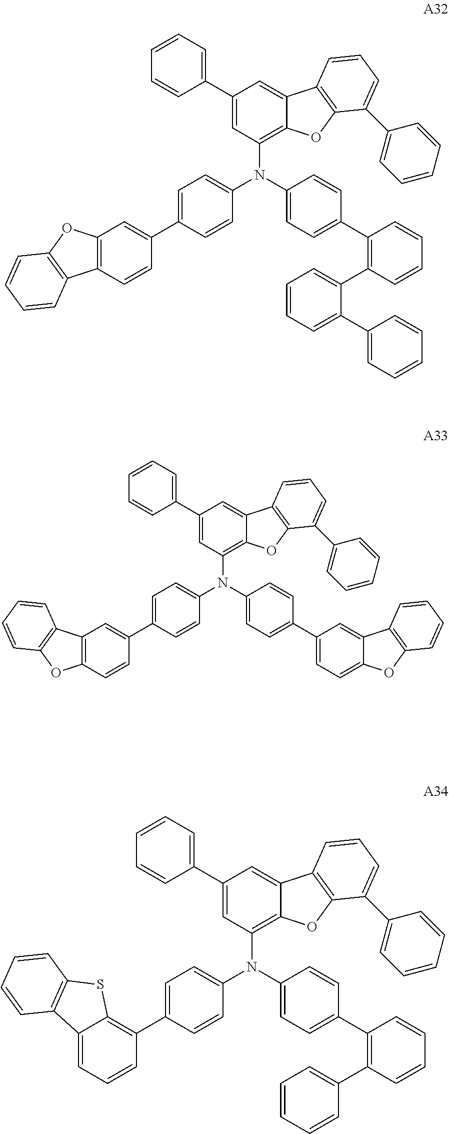 [SMILES: c1ccc(-c2cc(N(c3ccc(-c4ccc5c(c4)oc4ccccc45)cc3)c3ccc(-c4ccccc4-c4ccccc4-c4ccccc4)cc3)c3oc4c(-c5ccccc5)cccc4c3c2)cc1 ; c1ccc(-c2cc(N(c3ccc(-c4ccc5oc6ccccc6c5c4)cc3)c3ccc(-c4ccc5oc6ccccc6c5c4)cc3)c3oc4c(-c5ccccc5)cccc4c3c2)cc1 ; c1ccc(-c2cc(N(c3ccc(-c4ccccc4-c4ccccc4)cc3)c3ccc(-c4cccc5c4sc4ccccc45)cc3)c3oc4c(-c5ccccc5)cccc4c3c2)cc1] 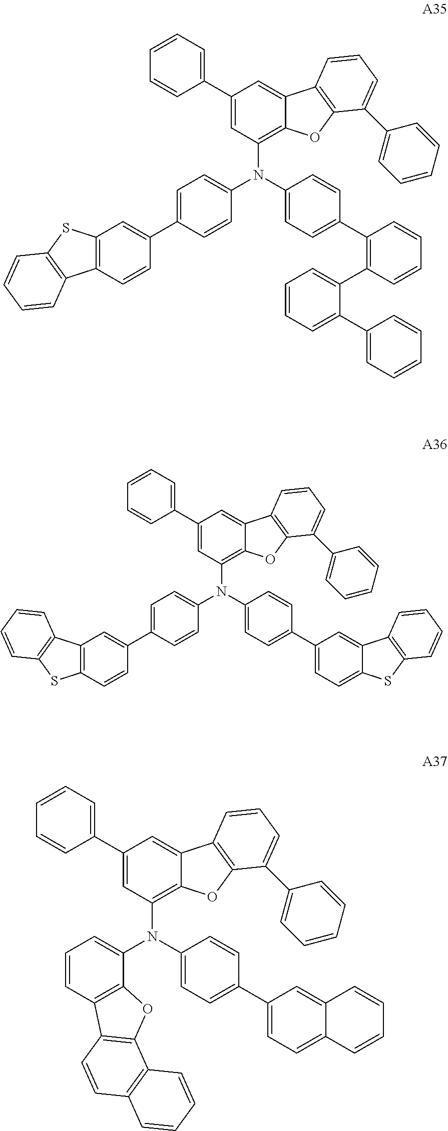 [SMILES: c1ccc(-c2cc(N(c3ccc(-c4ccc5c(c4)sc4ccccc45)cc3)c3ccc(-c4ccccc4-c4ccccc4-c4ccccc4)cc3)c3oc4c(-c5ccccc5)cccc4c3c2)cc1 ; c1ccc(-c2cc(N(c3ccc(-c4ccc5sc6ccccc6c5c4)cc3)c3ccc(-c4ccc5sc6ccccc6c5c4)cc3)c3oc4c(-c5ccccc5)cccc4c3c2)cc1 ; c1ccc(-c2cc(N(c3ccc(-c4ccc5ccccc5c4)cc3)c3cccc4c3oc3c5ccccc5ccc43)c3oc4c(-c5ccccc5)cccc4c3c2)cc1] 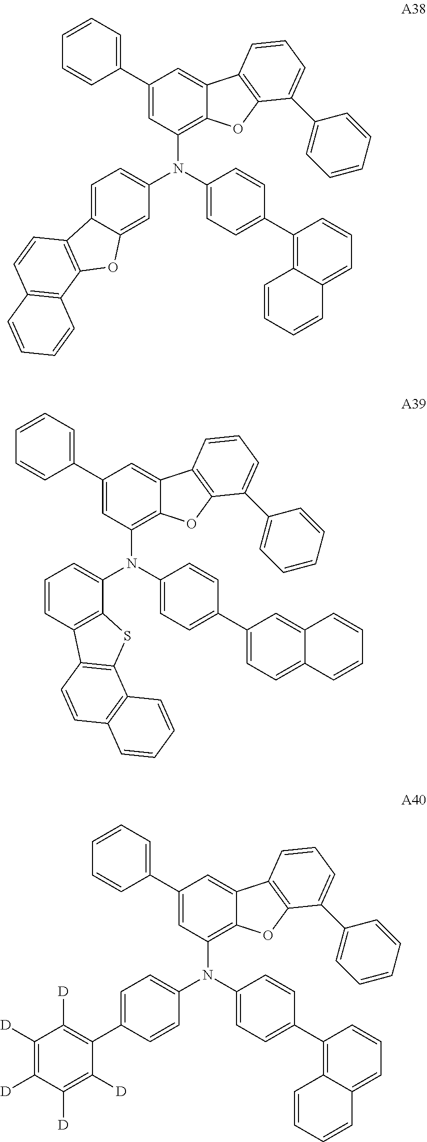 [SMILES: c1ccc(-c2cc(N(c3ccc(-c4cccc5ccccc45)cc3)c3ccc4c(c3)oc3c5ccccc5ccc43)c3oc4c(-c5ccccc5)cccc4c3c2)cc1 ; c1ccc(-c2cc(N(c3ccc(-c4ccc5ccccc5c4)cc3)c3cccc4c3sc3c5ccccc5ccc43)c3oc4c(-c5ccccc5)cccc4c3c2)cc1 ; [2H]c1c([2H])c([2H])c(-c2ccc(N(c3ccc(-c4cccc5ccccc45)cc3)c3cc(-c4ccccc4)cc4c3oc3c(-c5ccccc5)cccc34)cc2)c([2H])c1[2H]] 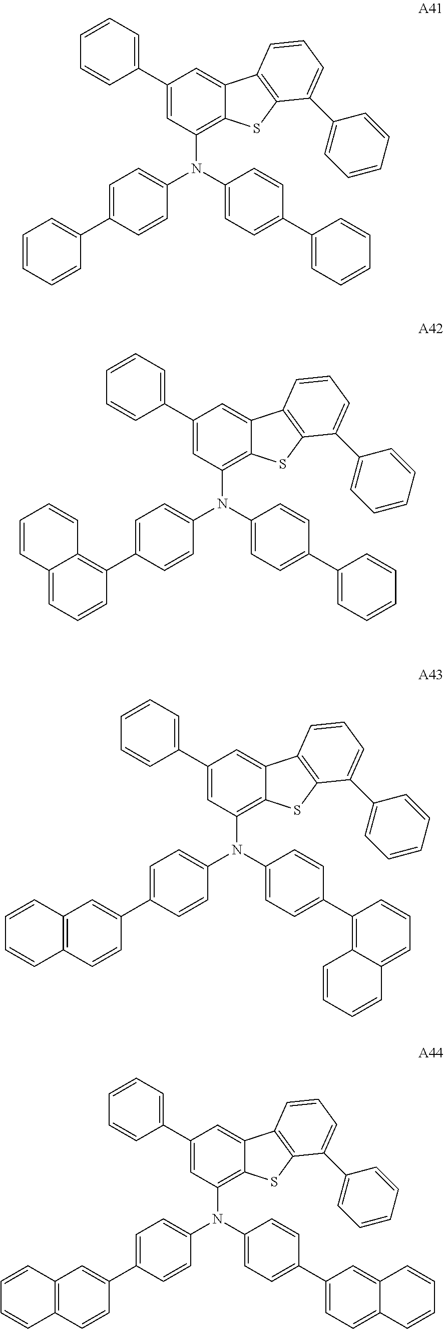 [SMILES: c1ccc(-c2ccc(N(c3ccc(-c4ccccc4)cc3)c3cc(-c4ccccc4)cc4c3sc3c(-c5ccccc5)cccc34)cc2)cc1 ; c1ccc(-c2ccc(N(c3ccc(-c4cccc5ccccc45)cc3)c3cc(-c4ccccc4)cc4c3sc3c(-c5ccccc5)cccc34)cc2)cc1 ; c1ccc(-c2cc(N(c3ccc(-c4ccc5ccccc5c4)cc3)c3ccc(-c4cccc5ccccc45)cc3)c3sc4c(-c5ccccc5)cccc4c3c2)cc1 ; c1ccc(-c2cc(N(c3ccc(-c4ccc5ccccc5c4)cc3)c3ccc(-c4ccc5ccccc5c4)cc3)c3sc4c(-c5ccccc5)cccc4c3c2)cc1] 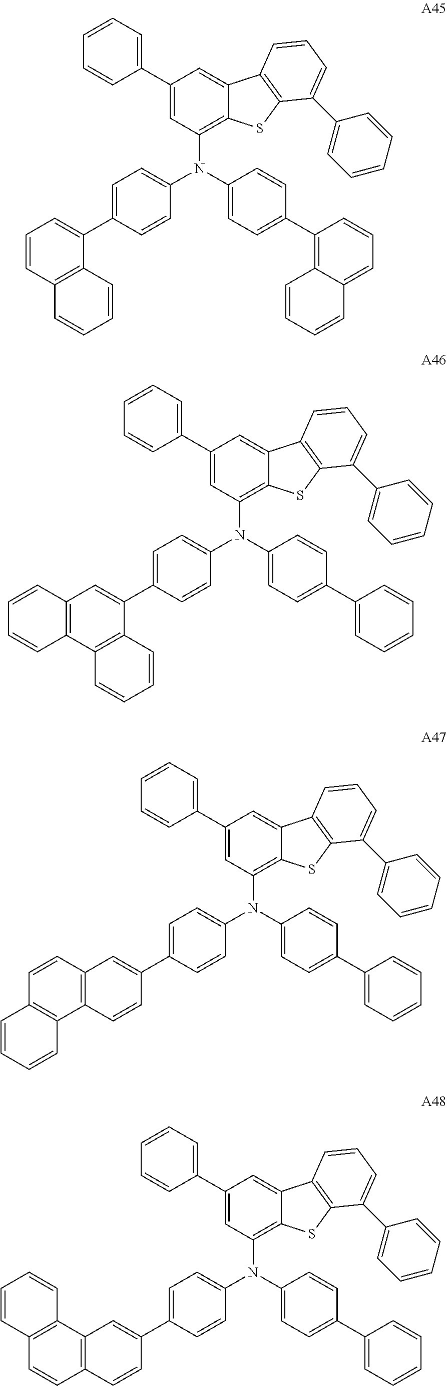 [SMILES: c1ccc(-c2cc(N(c3ccc(-c4cccc5ccccc45)cc3)c3ccc(-c4cccc5ccccc45)cc3)c3sc4c(-c5ccccc5)cccc4c3c2)cc1 ; c1ccc(-c2ccc(N(c3ccc(-c4cc5ccccc5c5ccccc45)cc3)c3cc(-c4ccccc4)cc4c3sc3c(-c5ccccc5)cccc34)cc2)cc1 ; c1ccc(-c2ccc(N(c3ccc(-c4ccc5c(ccc6ccccc65)c4)cc3)c3cc(-c4ccccc4)cc4c3sc3c(-c5ccccc5)cccc34)cc2)cc1 ; c1ccc(-c2ccc(N(c3ccc(-c4ccc5ccc6ccccc6c5c4)cc3)c3cc(-c4ccccc4)cc4c3sc3c(-c5ccccc5)cccc34)cc2)cc1] 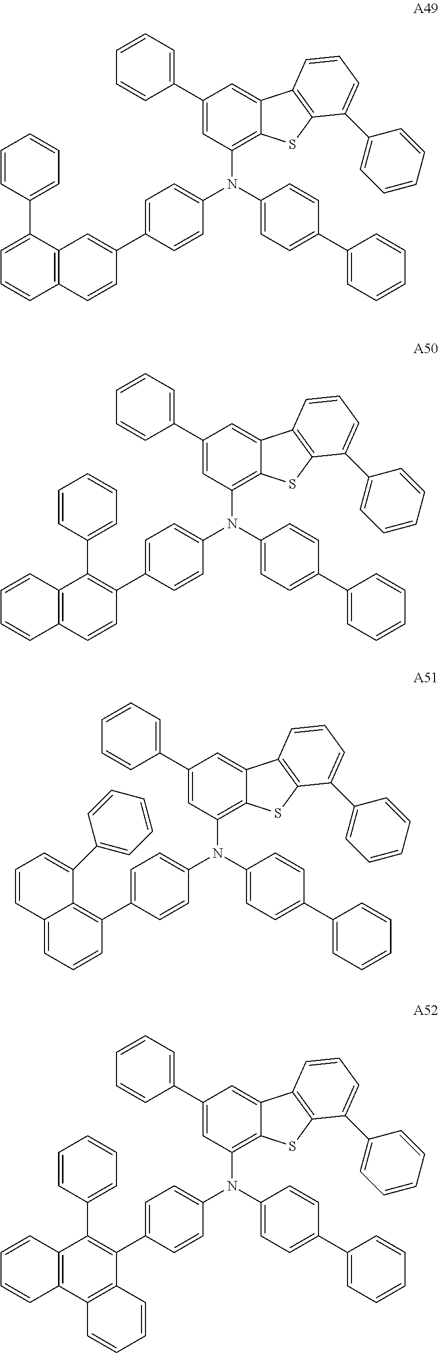 [SMILES: c1ccc(-c2ccc(N(c3ccc(-c4ccc5cccc(-c6ccccc6)c5c4)cc3)c3cc(-c4ccccc4)cc4c3sc3c(-c5ccccc5)cccc34)cc2)cc1 ; c1ccc(-c2ccc(N(c3ccc(-c4ccc5ccccc5c4-c4ccccc4)cc3)c3cc(-c4ccccc4)cc4c3sc3c(-c5ccccc5)cccc34)cc2)cc1 ; c1ccc(-c2ccc(N(c3ccc(-c4cccc5cccc(-c6ccccc6)c45)cc3)c3cc(-c4ccccc4)cc4c3sc3c(-c5ccccc5)cccc34)cc2)cc1 ; c1ccc(-c2ccc(N(c3ccc(-c4c(-c5ccccc5)c5ccccc5c5ccccc45)cc3)c3cc(-c4ccccc4)cc4c3sc3c(-c5ccccc5)cccc34)cc2)cc1] 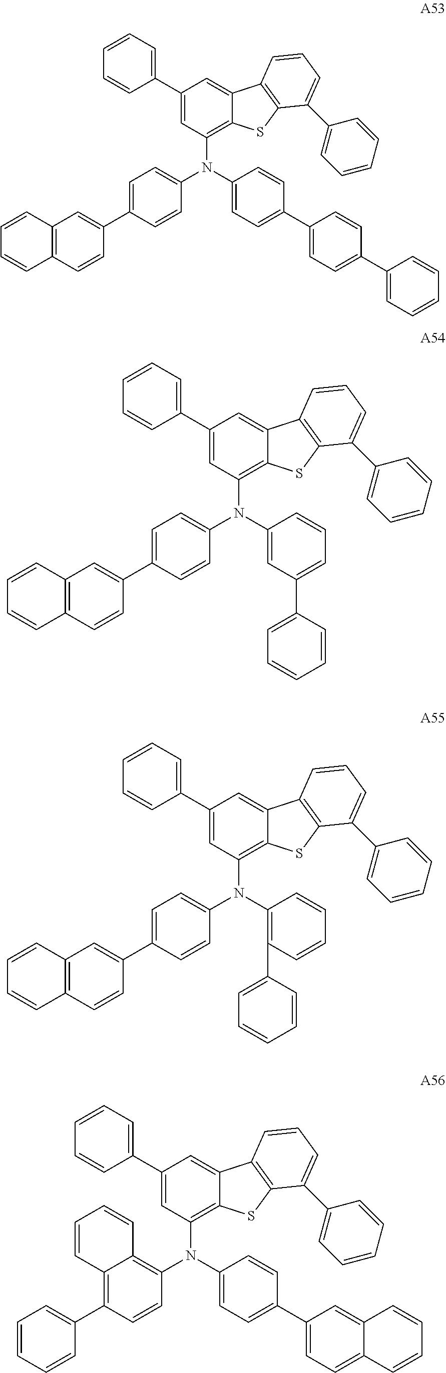 [SMILES: c1ccc(-c2ccc(-c3ccc(N(c4ccc(-c5ccc6ccccc6c5)cc4)c4cc(-c5ccccc5)cc5c4sc4c(-c6ccccc6)cccc45)cc3)cc2)cc1 ; c1ccc(-c2cccc(N(c3ccc(-c4ccc5ccccc5c4)cc3)c3cc(-c4ccccc4)cc4c3sc3c(-c5ccccc5)cccc34)c2)cc1 ; c1ccc(-c2cc(N(c3ccc(-c4ccc5ccccc5c4)cc3)c3ccccc3-c3ccccc3)c3sc4c(-c5ccccc5)cccc4c3c2)cc1 ; c1ccc(-c2cc(N(c3ccc(-c4ccc5ccccc5c4)cc3)c3ccc(-c4ccccc4)c4ccccc34)c3sc4c(-c5ccccc5)cccc4c3c2)cc1] 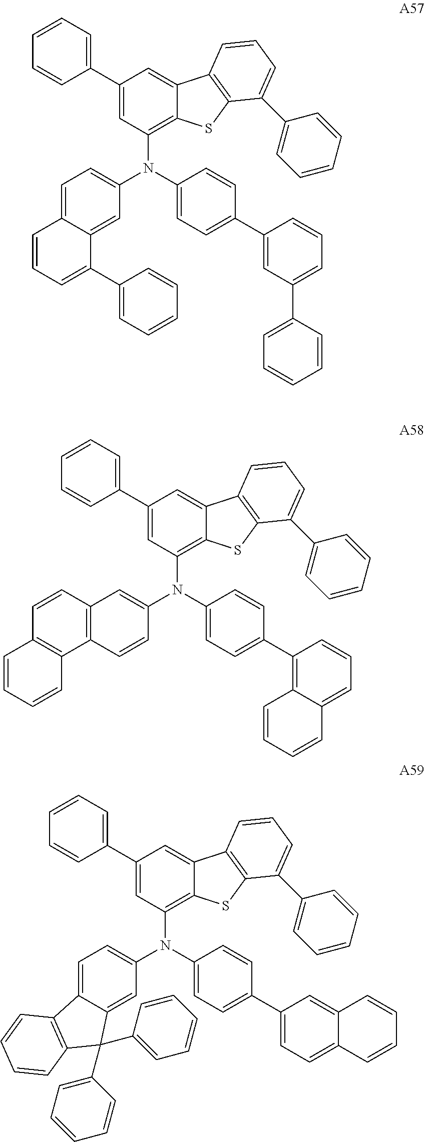 [SMILES: c1ccc(-c2cccc(-c3ccc(N(c4ccc5cccc(-c6ccccc6)c5c4)c4cc(-c5ccccc5)cc5c4sc4c(-c6ccccc6)cccc45)cc3)c2)cc1 ; c1ccc(-c2cc(N(c3ccc(-c4cccc5ccccc45)cc3)c3ccc4c(ccc5ccccc54)c3)c3sc4c(-c5ccccc5)cccc4c3c2)cc1 ; c1ccc(-c2cc(N(c3ccc(-c4ccc5ccccc5c4)cc3)c3ccc4c(c3)C(c3ccccc3)(c3ccccc3)c3ccccc3-4)c3sc4c(-c5ccccc5)cccc4c3c2)cc1] 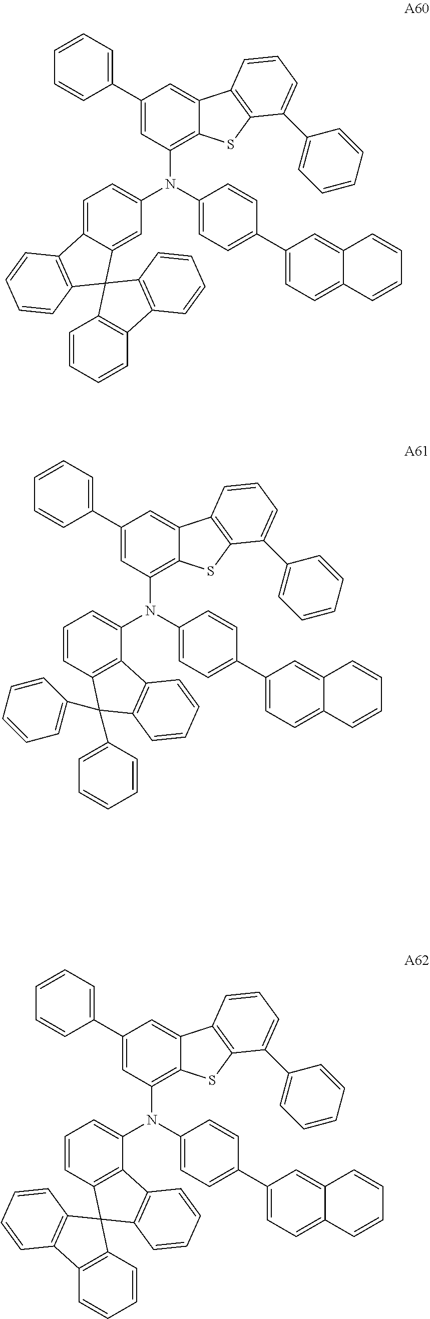 [SMILES: c1ccc(-c2cc(N(c3ccc(-c4ccc5ccccc5c4)cc3)c3ccc4c(c3)C3(c5ccccc5-c5ccccc53)c3ccccc3-4)c3sc4c(-c5ccccc5)cccc4c3c2)cc1 ; c1ccc(-c2cc(N(c3ccc(-c4ccc5ccccc5c4)cc3)c3cccc4c3-c3ccccc3C4(c3ccccc3)c3ccccc3)c3sc4c(-c5ccccc5)cccc4c3c2)cc1 ; c1ccc(-c2cc(N(c3ccc(-c4ccc5ccccc5c4)cc3)c3cccc4c3-c3ccccc3C43c4ccccc4-c4ccccc43)c3sc4c(-c5ccccc5)cccc4c3c2)cc1] 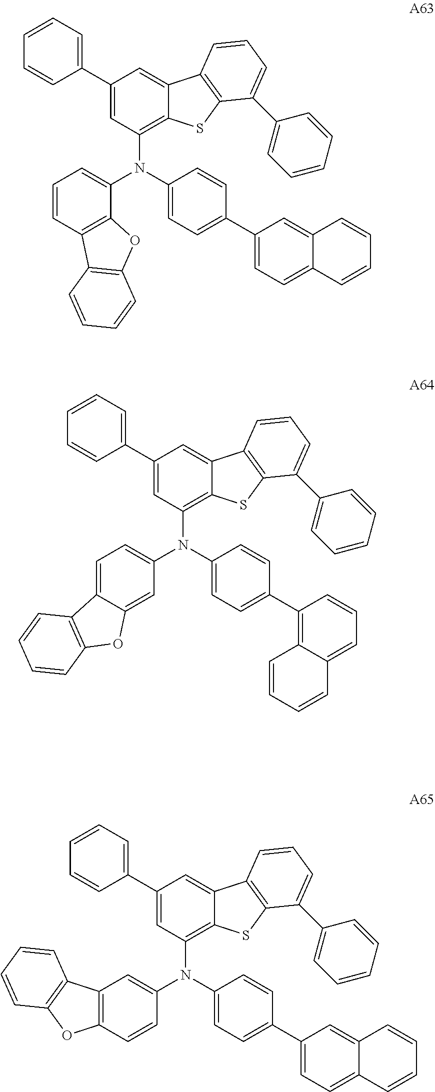 [SMILES: c1ccc(-c2cc(N(c3ccc(-c4ccc5ccccc5c4)cc3)c3cccc4c3oc3ccccc34)c3sc4c(-c5ccccc5)cccc4c3c2)cc1 ; c1ccc(-c2cc(N(c3ccc(-c4cccc5ccccc45)cc3)c3ccc4c(c3)oc3ccccc34)c3sc4c(-c5ccccc5)cccc4c3c2)cc1 ; c1ccc(-c2cc(N(c3ccc(-c4ccc5ccccc5c4)cc3)c3ccc4oc5ccccc5c4c3)c3sc4c(-c5ccccc5)cccc4c3c2)cc1] 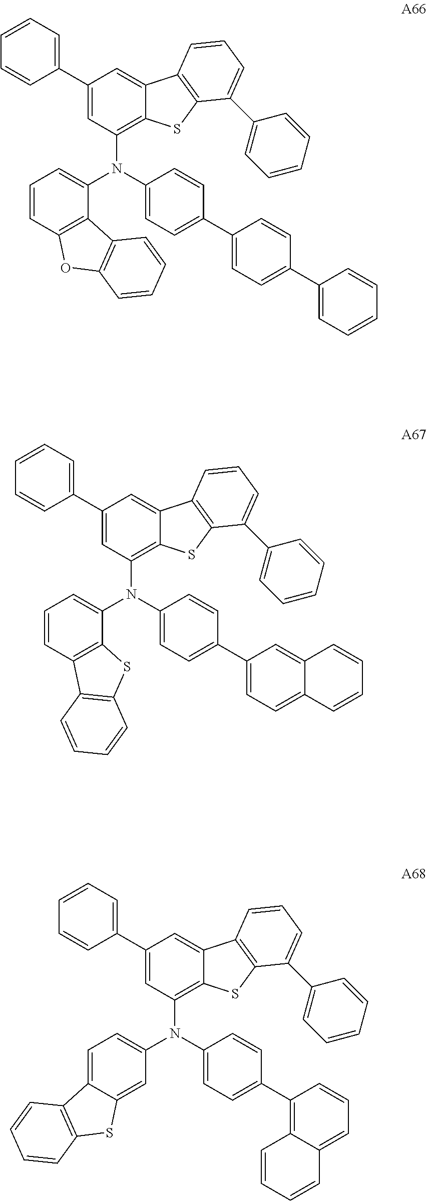 [SMILES: c1ccc(-c2ccc(-c3ccc(N(c4cc(-c5ccccc5)cc5c4sc4c(-c6ccccc6)cccc45)c4cccc5oc6ccccc6c45)cc3)cc2)cc1 ; c1ccc(-c2cc(N(c3ccc(-c4ccc5ccccc5c4)cc3)c3cccc4c3sc3ccccc34)c3sc4c(-c5ccccc5)cccc4c3c2)cc1 ; c1ccc(-c2cc(N(c3ccc(-c4cccc5ccccc45)cc3)c3ccc4c(c3)sc3ccccc34)c3sc4c(-c5ccccc5)cccc4c3c2)cc1] 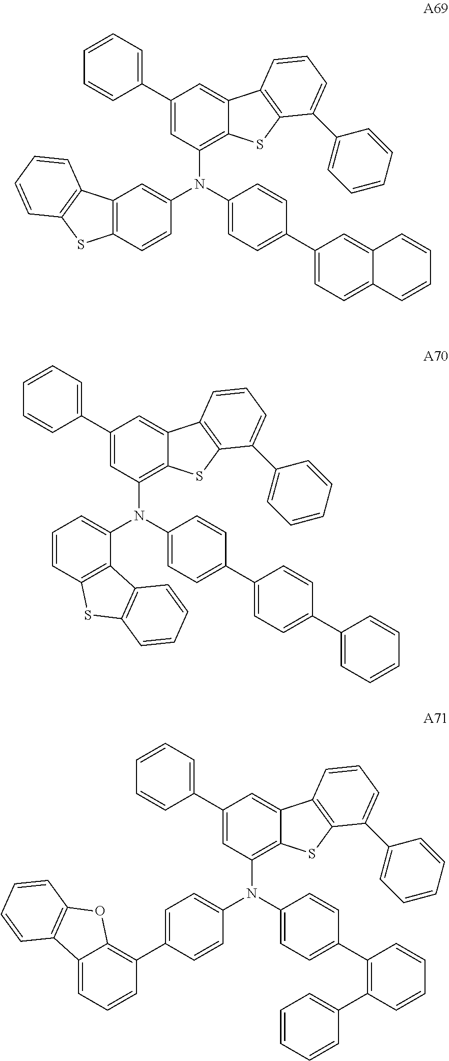 [SMILES: c1ccc(-c2cc(N(c3ccc(-c4ccc5ccccc5c4)cc3)c3ccc4sc5ccccc5c4c3)c3sc4c(-c5ccccc5)cccc4c3c2)cc1 ; c1ccc(-c2ccc(-c3ccc(N(c4cc(-c5ccccc5)cc5c4sc4c(-c6ccccc6)cccc45)c4cccc5sc6ccccc6c45)cc3)cc2)cc1 ; c1ccc(-c2cc(N(c3ccc(-c4ccccc4-c4ccccc4)cc3)c3ccc(-c4cccc5c4oc4ccccc45)cc3)c3sc4c(-c5ccccc5)cccc4c3c2)cc1] 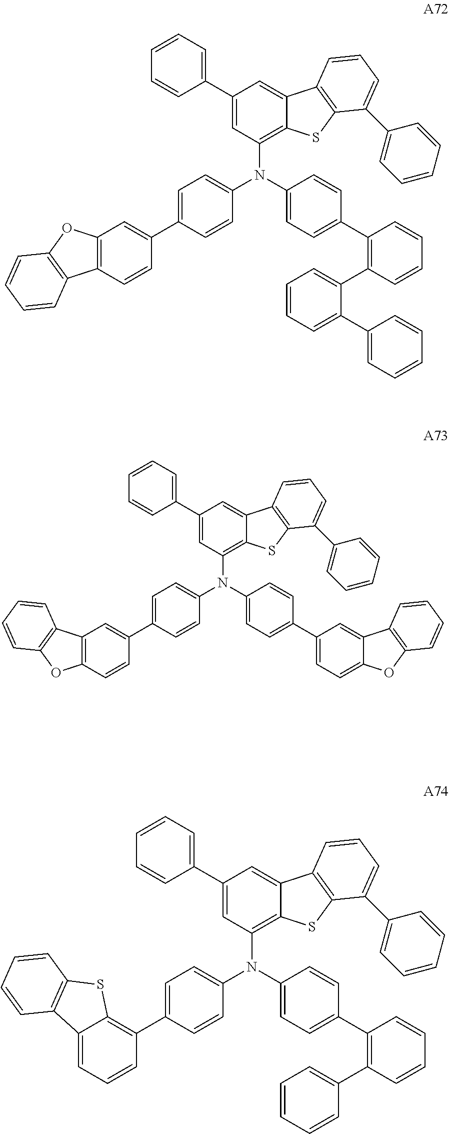 [SMILES: c1ccc(-c2cc(N(c3ccc(-c4ccc5c(c4)oc4ccccc45)cc3)c3ccc(-c4ccccc4-c4ccccc4-c4ccccc4)cc3)c3sc4c(-c5ccccc5)cccc4c3c2)cc1 ; c1ccc(-c2cc(N(c3ccc(-c4ccc5oc6ccccc6c5c4)cc3)c3ccc(-c4ccc5oc6ccccc6c5c4)cc3)c3sc4c(-c5ccccc5)cccc4c3c2)cc1 ; c1ccc(-c2cc(N(c3ccc(-c4ccccc4-c4ccccc4)cc3)c3ccc(-c4cccc5c4sc4ccccc45)cc3)c3sc4c(-c5ccccc5)cccc4c3c2)cc1] 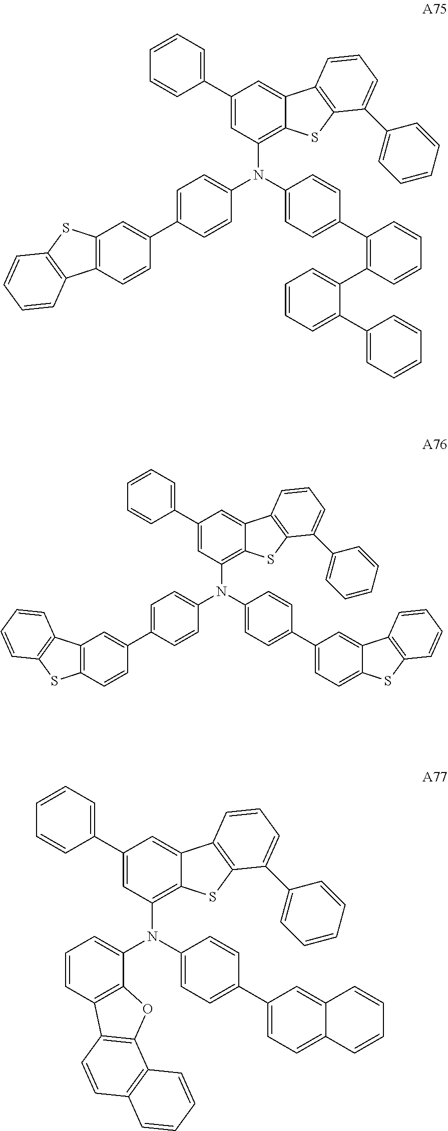 [SMILES: c1ccc(-c2cc(N(c3ccc(-c4ccc5c(c4)sc4ccccc45)cc3)c3ccc(-c4ccccc4-c4ccccc4-c4ccccc4)cc3)c3sc4c(-c5ccccc5)cccc4c3c2)cc1 ; c1ccc(-c2cc(N(c3ccc(-c4ccc5sc6ccccc6c5c4)cc3)c3ccc(-c4ccc5sc6ccccc6c5c4)cc3)c3sc4c(-c5ccccc5)cccc4c3c2)cc1 ; c1ccc(-c2cc(N(c3ccc(-c4ccc5ccccc5c4)cc3)c3cccc4c3oc3c5ccccc5ccc43)c3sc4c(-c5ccccc5)cccc4c3c2)cc1] 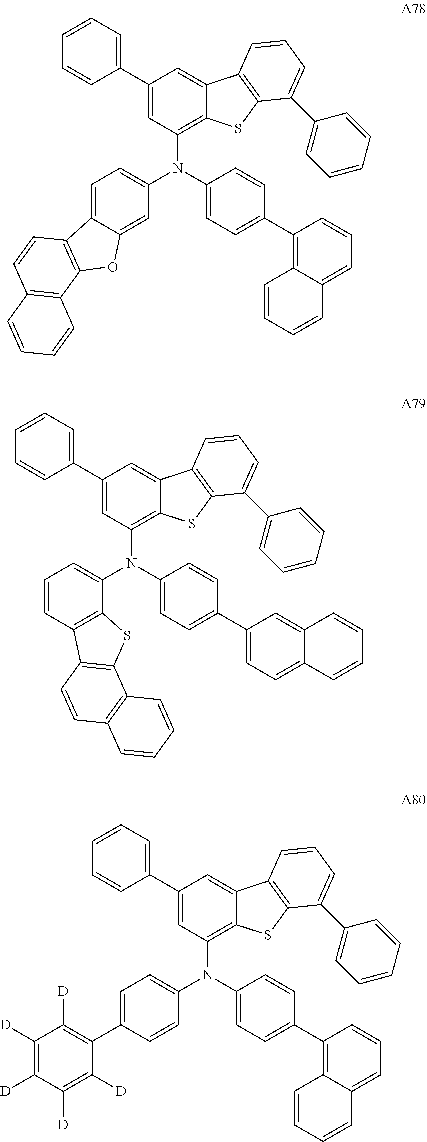 [SMILES: c1ccc(-c2cc(N(c3ccc(-c4cccc5ccccc45)cc3)c3ccc4c(c3)oc3c5ccccc5ccc43)c3sc4c(-c5ccccc5)cccc4c3c2)cc1 ; c1ccc(-c2cc(N(c3ccc(-c4ccc5ccccc5c4)cc3)c3cccc4c3sc3c5ccccc5ccc43)c3sc4c(-c5ccccc5)cccc4c3c2)cc1 ; [2H]c1c([2H])c([2H])c(-c2ccc(N(c3ccc(-c4cccc5ccccc45)cc3)c3cc(-c4ccccc4)cc4c3sc3c(-c5ccccc5)cccc34)cc2)c([2H])c1[2H]] 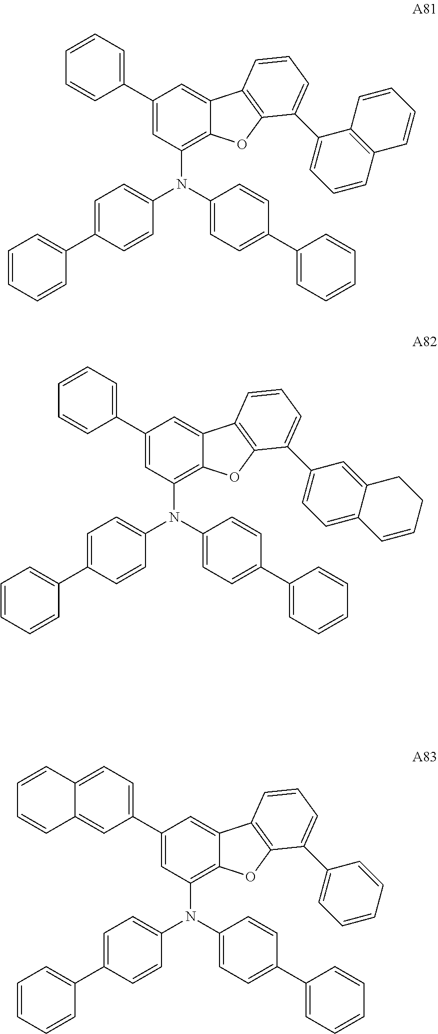 [SMILES: c1ccc(-c2ccc(N(c3ccc(-c4ccccc4)cc3)c3cc(-c4ccccc4)cc4c3oc3c(-c5cccc6ccccc56)cccc34)cc2)cc1 ; C1=Cc2ccc(-c3cccc4c3oc3c(N(c5ccc(-c6ccccc6)cc5)c5ccc(-c6ccccc6)cc5)cc(-c5ccccc5)cc34)cc2CC1 ; c1ccc(-c2ccc(N(c3ccc(-c4ccccc4)cc3)c3cc(-c4ccc5ccccc5c4)cc4c3oc3c(-c5ccccc5)cccc34)cc2)cc1] 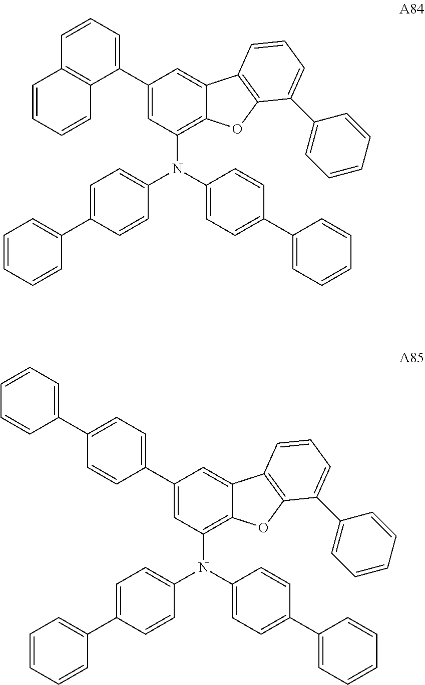 [SMILES: c1ccc(-c2ccc(N(c3ccc(-c4ccccc4)cc3)c3cc(-c4cccc5ccccc45)cc4c3oc3c(-c5ccccc5)cccc34)cc2)cc1 ; c1ccc(-c2ccc(-c3cc(N(c4ccc(-c5ccccc5)cc4)c4ccc(-c5ccccc5)cc4)c4oc5c(-c6ccccc6)cccc5c4c3)cc2)cc1] 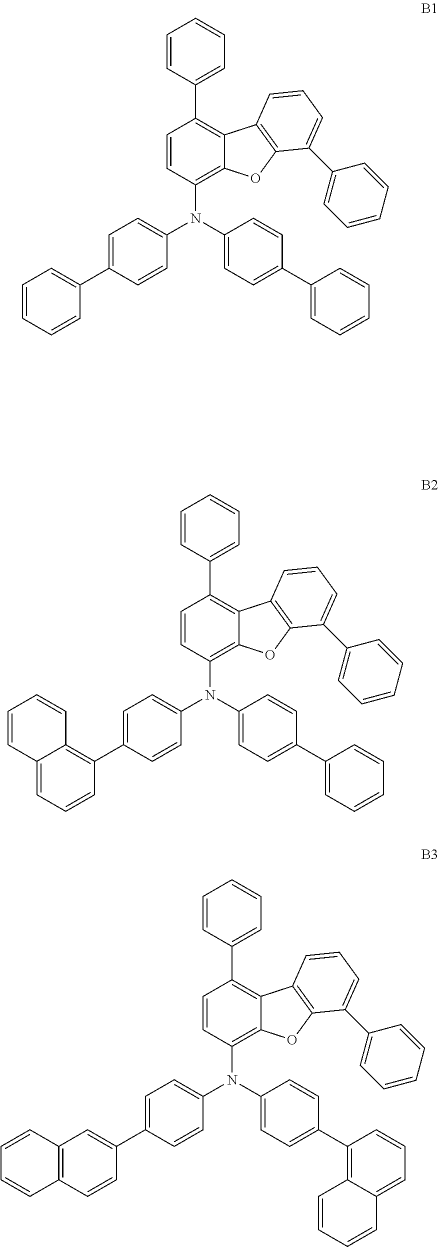 [SMILES: c1ccc(-c2ccc(N(c3ccc(-c4ccccc4)cc3)c3ccc(-c4ccccc4)c4c3oc3c(-c5ccccc5)cccc34)cc2)cc1 ; c1ccc(-c2ccc(N(c3ccc(-c4cccc5ccccc45)cc3)c3ccc(-c4ccccc4)c4c3oc3c(-c5ccccc5)cccc34)cc2)cc1 ; c1ccc(-c2cccc3c2oc2c(N(c4ccc(-c5ccc6ccccc6c5)cc4)c4ccc(-c5cccc6ccccc56)cc4)ccc(-c4ccccc4)c23)cc1] 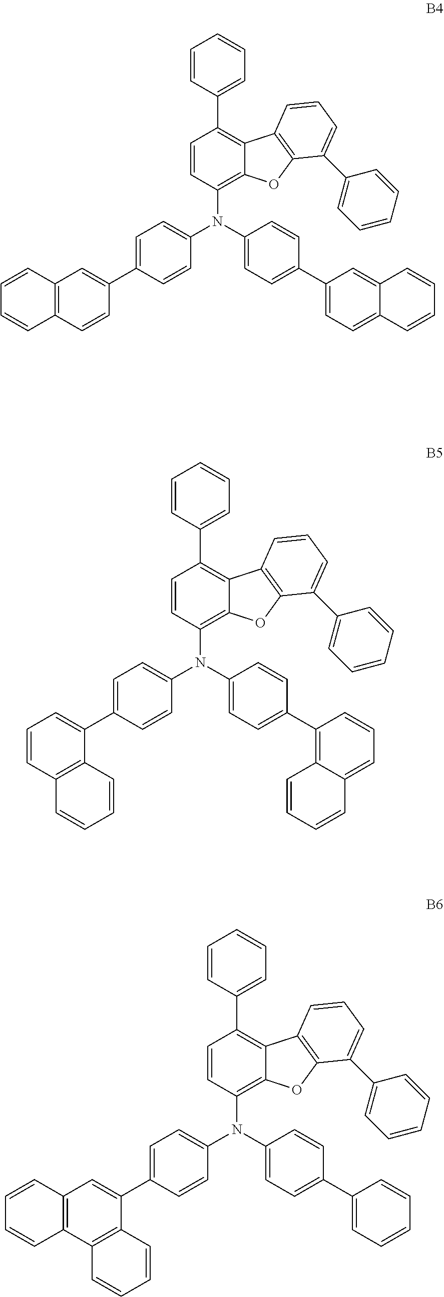 [SMILES: c1ccc(-c2cccc3c2oc2c(N(c4ccc(-c5ccc6ccccc6c5)cc4)c4ccc(-c5ccc6ccccc6c5)cc4)ccc(-c4ccccc4)c23)cc1 ; c1ccc(-c2cccc3c2oc2c(N(c4ccc(-c5cccc6ccccc56)cc4)c4ccc(-c5cccc6ccccc56)cc4)ccc(-c4ccccc4)c23)cc1 ; c1ccc(-c2ccc(N(c3ccc(-c4cc5ccccc5c5ccccc45)cc3)c3ccc(-c4ccccc4)c4c3oc3c(-c5ccccc5)cccc34)cc2)cc1] 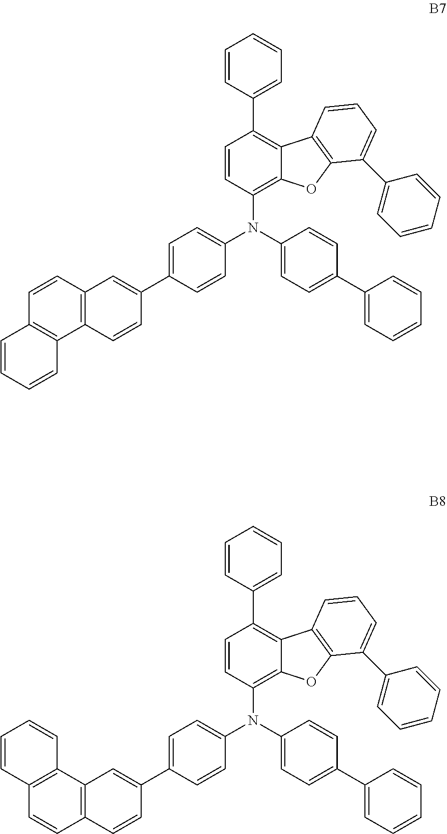 [SMILES: c1ccc(-c2ccc(N(c3ccc(-c4ccc5c(ccc6ccccc65)c4)cc3)c3ccc(-c4ccccc4)c4c3oc3c(-c5ccccc5)cccc34)cc2)cc1 ; c1ccc(-c2ccc(N(c3ccc(-c4ccc5ccc6ccccc6c5c4)cc3)c3ccc(-c4ccccc4)c4c3oc3c(-c5ccccc5)cccc34)cc2)cc1] 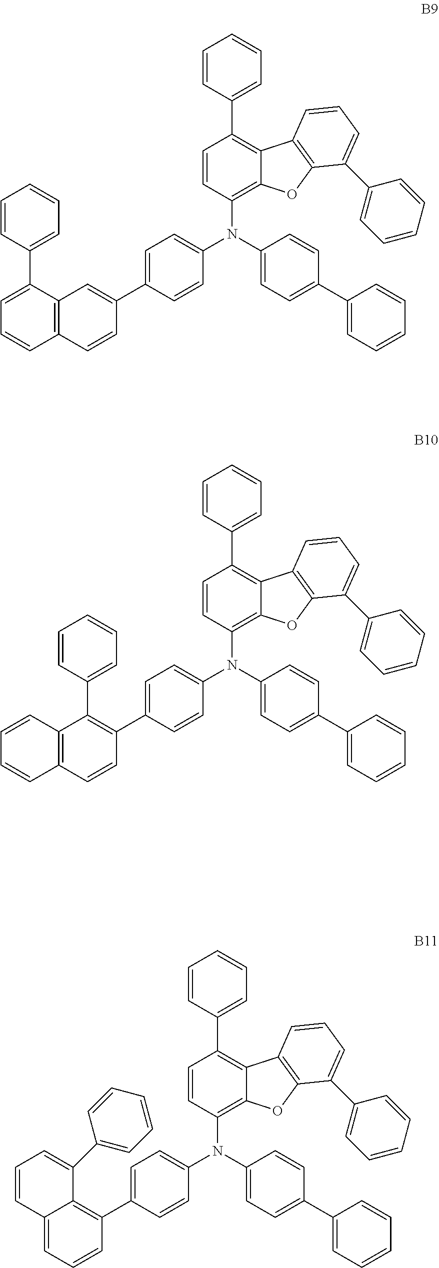 [SMILES: c1ccc(-c2ccc(N(c3ccc(-c4ccc5cccc(-c6ccccc6)c5c4)cc3)c3ccc(-c4ccccc4)c4c3oc3c(-c5ccccc5)cccc34)cc2)cc1 ; c1ccc(-c2ccc(N(c3ccc(-c4ccc5ccccc5c4-c4ccccc4)cc3)c3ccc(-c4ccccc4)c4c3oc3c(-c5ccccc5)cccc34)cc2)cc1 ; c1ccc(-c2ccc(N(c3ccc(-c4cccc5cccc(-c6ccccc6)c45)cc3)c3ccc(-c4ccccc4)c4c3oc3c(-c5ccccc5)cccc34)cc2)cc1] 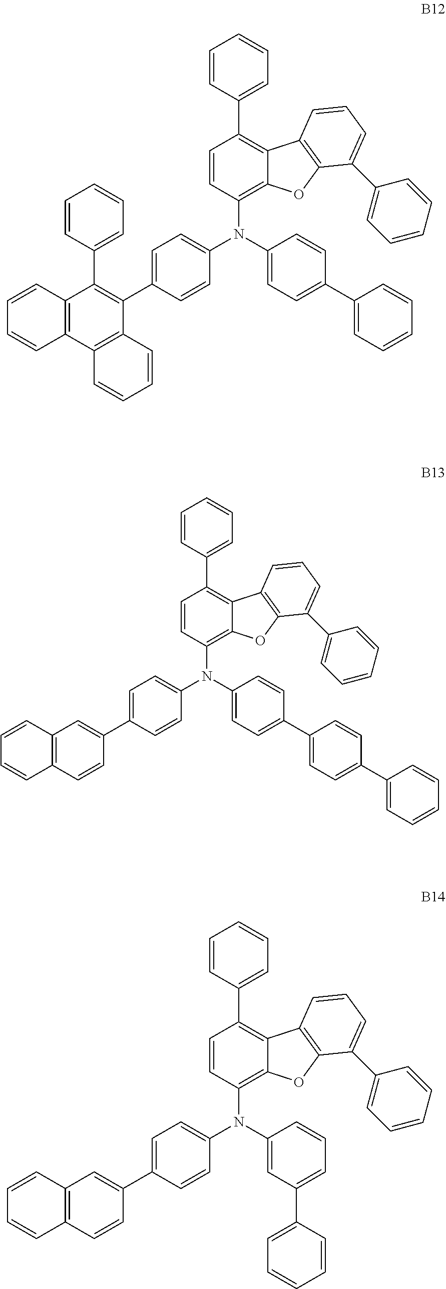 [SMILES: c1ccc(-c2ccc(N(c3ccc(-c4c(-c5ccccc5)c5ccccc5c5ccccc45)cc3)c3ccc(-c4ccccc4)c4c3oc3c(-c5ccccc5)cccc34)cc2)cc1 ; c1ccc(-c2ccc(-c3ccc(N(c4ccc(-c5ccc6ccccc6c5)cc4)c4ccc(-c5ccccc5)c5c4oc4c(-c6ccccc6)cccc45)cc3)cc2)cc1 ; c1ccc(-c2cccc(N(c3ccc(-c4ccc5ccccc5c4)cc3)c3ccc(-c4ccccc4)c4c3oc3c(-c5ccccc5)cccc34)c2)cc1] 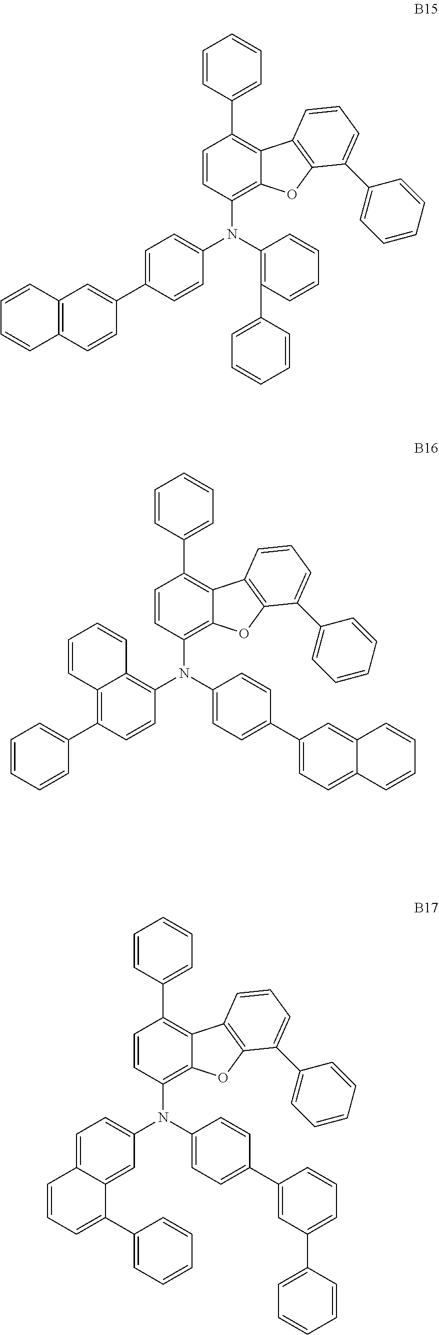 [SMILES: c1ccc(-c2ccccc2N(c2ccc(-c3ccc4ccccc4c3)cc2)c2ccc(-c3ccccc3)c3c2oc2c(-c4ccccc4)cccc23)cc1 ; c1ccc(-c2ccc(N(c3ccc(-c4ccc5ccccc5c4)cc3)c3ccc(-c4ccccc4)c4c3oc3c(-c5ccccc5)cccc34)c3ccccc23)cc1 ; c1ccc(-c2cccc(-c3ccc(N(c4ccc5cccc(-c6ccccc6)c5c4)c4ccc(-c5ccccc5)c5c4oc4c(-c6ccccc6)cccc45)cc3)c2)cc1] 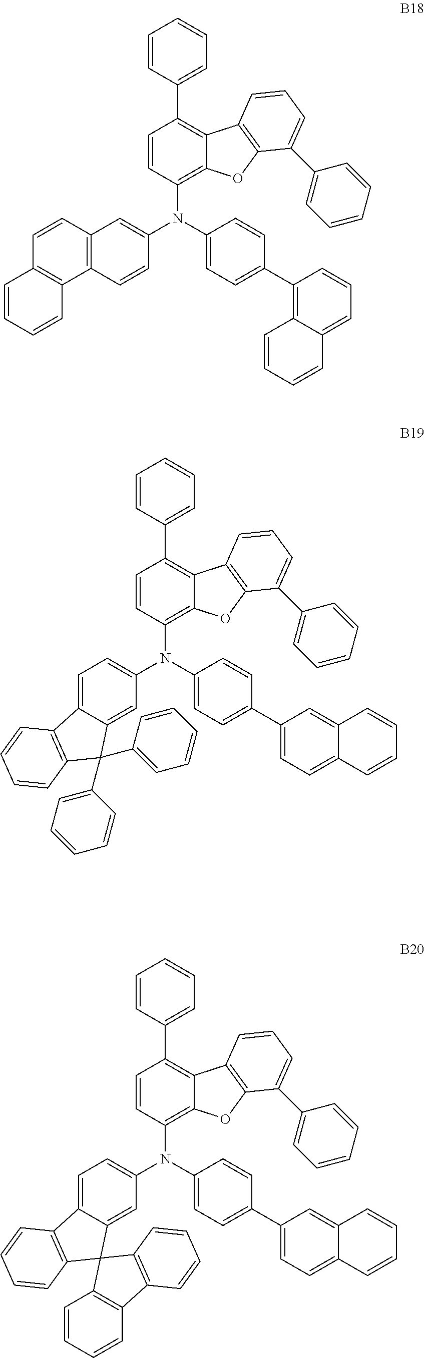 [SMILES: c1ccc(-c2cccc3c2oc2c(N(c4ccc(-c5cccc6ccccc56)cc4)c4ccc5c(ccc6ccccc65)c4)ccc(-c4ccccc4)c23)cc1 ; c1ccc(-c2cccc3c2oc2c(N(c4ccc(-c5ccc6ccccc6c5)cc4)c4ccc5c(c4)C(c4ccccc4)(c4ccccc4)c4ccccc4-5)ccc(-c4ccccc4)c23)cc1 ; c1ccc(-c2cccc3c2oc2c(N(c4ccc(-c5ccc6ccccc6c5)cc4)c4ccc5c(c4)C4(c6ccccc6-c6ccccc64)c4ccccc4-5)ccc(-c4ccccc4)c23)cc1] 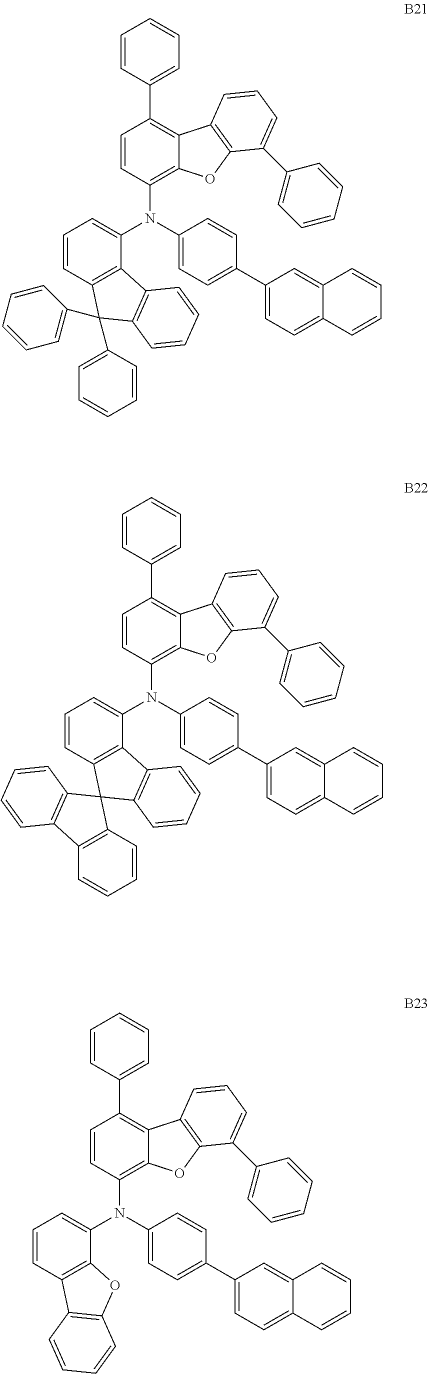 [SMILES: c1ccc(-c2cccc3c2oc2c(N(c4ccc(-c5ccc6ccccc6c5)cc4)c4cccc5c4-c4ccccc4C5(c4ccccc4)c4ccccc4)ccc(-c4ccccc4)c23)cc1 ; c1ccc(-c2cccc3c2oc2c(N(c4ccc(-c5ccc6ccccc6c5)cc4)c4cccc5c4-c4ccccc4C54c5ccccc5-c5ccccc54)ccc(-c4ccccc4)c23)cc1 ; c1ccc(-c2cccc3c2oc2c(N(c4ccc(-c5ccc6ccccc6c5)cc4)c4cccc5c4oc4ccccc45)ccc(-c4ccccc4)c23)cc1] 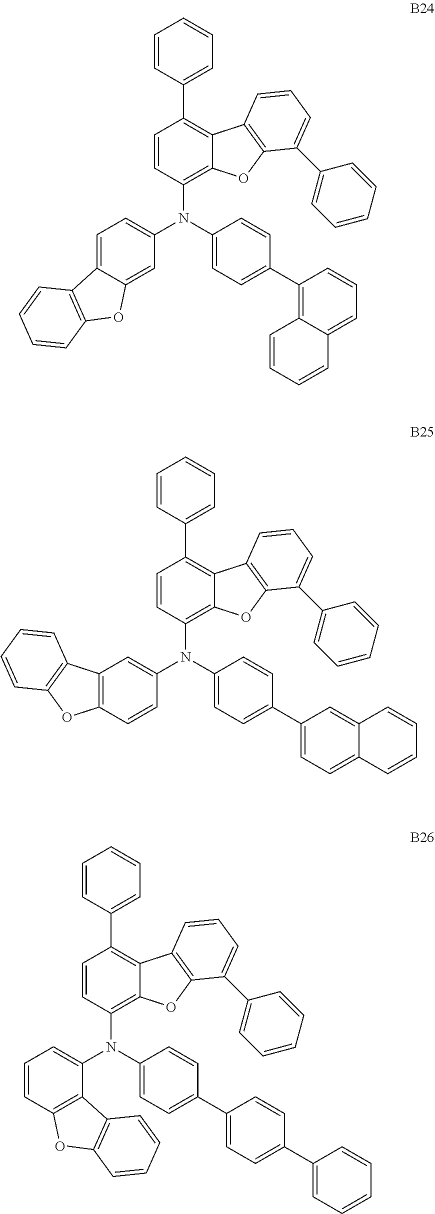 [SMILES: c1ccc(-c2cccc3c2oc2c(N(c4ccc(-c5cccc6ccccc56)cc4)c4ccc5c(c4)oc4ccccc45)ccc(-c4ccccc4)c23)cc1 ; c1ccc(-c2cccc3c2oc2c(N(c4ccc(-c5ccc6ccccc6c5)cc4)c4ccc5oc6ccccc6c5c4)ccc(-c4ccccc4)c23)cc1 ; c1ccc(-c2ccc(-c3ccc(N(c4ccc(-c5ccccc5)c5c4oc4c(-c6ccccc6)cccc45)c4cccc5oc6ccccc6c45)cc3)cc2)cc1] 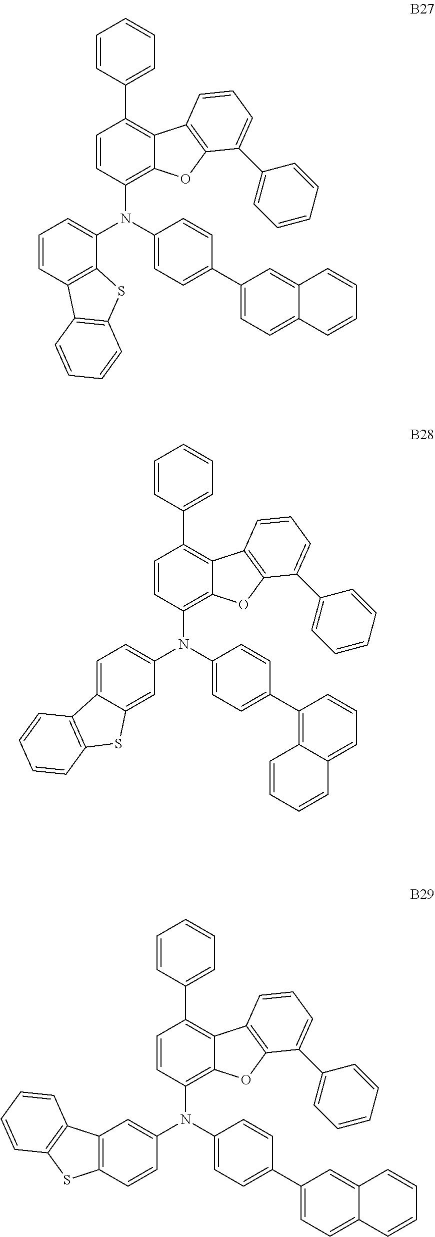 [SMILES: c1ccc(-c2cccc3c2oc2c(N(c4ccc(-c5ccc6ccccc6c5)cc4)c4cccc5c4sc4ccccc45)ccc(-c4ccccc4)c23)cc1 ; c1ccc(-c2cccc3c2oc2c(N(c4ccc(-c5cccc6ccccc56)cc4)c4ccc5c(c4)sc4ccccc45)ccc(-c4ccccc4)c23)cc1 ; c1ccc(-c2cccc3c2oc2c(N(c4ccc(-c5ccc6ccccc6c5)cc4)c4ccc5sc6ccccc6c5c4)ccc(-c4ccccc4)c23)cc1] 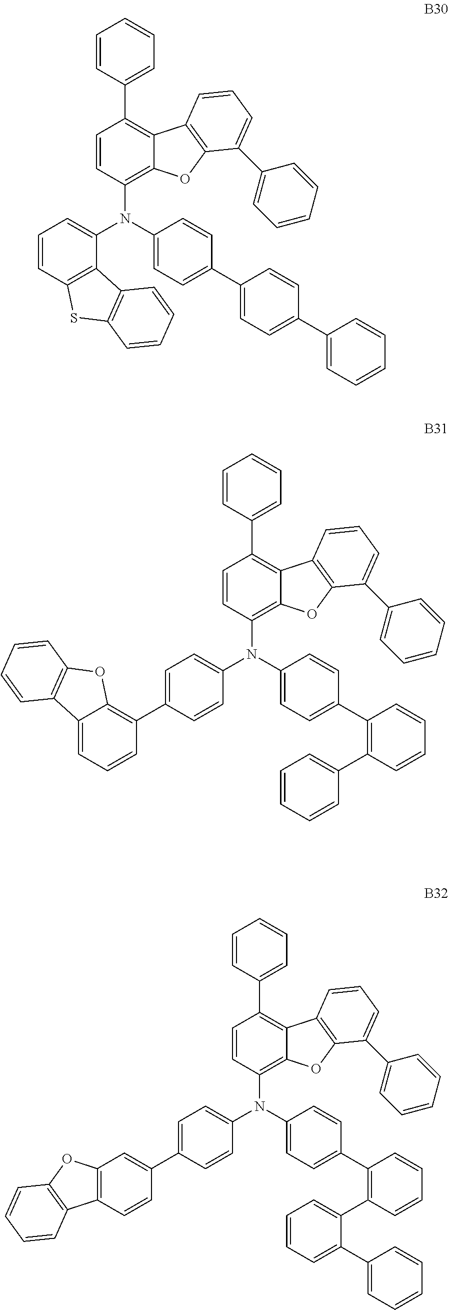 [SMILES: c1ccc(-c2ccc(-c3ccc(N(c4ccc(-c5ccccc5)c5c4oc4c(-c6ccccc6)cccc45)c4cccc5sc6ccccc6c45)cc3)cc2)cc1 ; c1ccc(-c2ccccc2-c2ccc(N(c3ccc(-c4cccc5c4oc4ccccc45)cc3)c3ccc(-c4ccccc4)c4c3oc3c(-c5ccccc5)cccc34)cc2)cc1 ; c1ccc(-c2ccccc2-c2ccccc2-c2ccc(N(c3ccc(-c4ccc5c(c4)oc4ccccc45)cc3)c3ccc(-c4ccccc4)c4c3oc3c(-c5ccccc5)cccc34)cc2)cc1] 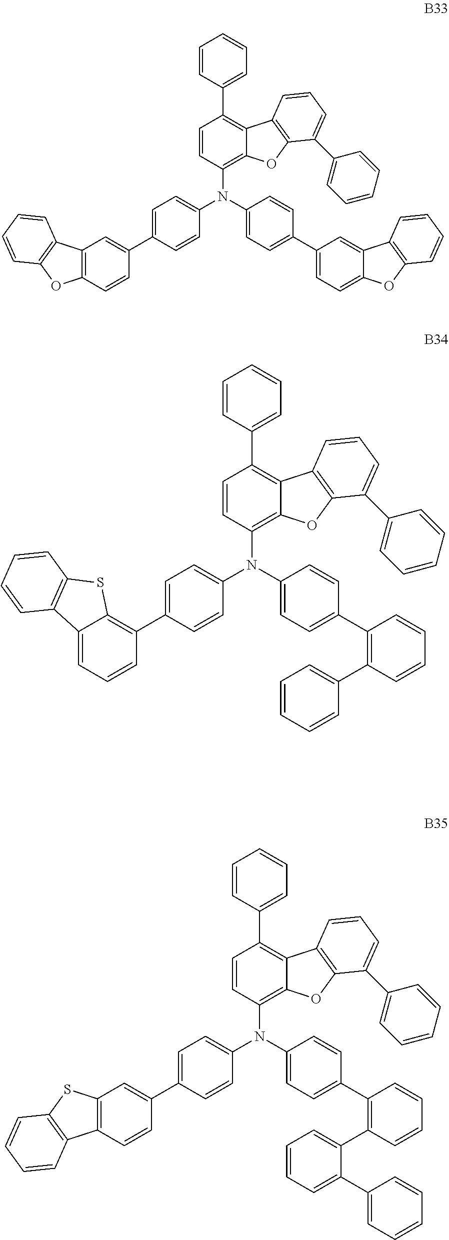 [SMILES: c1ccc(-c2cccc3c2oc2c(N(c4ccc(-c5ccc6oc7ccccc7c6c5)cc4)c4ccc(-c5ccc6oc7ccccc7c6c5)cc4)ccc(-c4ccccc4)c23)cc1 ; c1ccc(-c2ccccc2-c2ccc(N(c3ccc(-c4cccc5c4sc4ccccc45)cc3)c3ccc(-c4ccccc4)c4c3oc3c(-c5ccccc5)cccc34)cc2)cc1 ; c1ccc(-c2ccccc2-c2ccccc2-c2ccc(N(c3ccc(-c4ccc5c(c4)sc4ccccc45)cc3)c3ccc(-c4ccccc4)c4c3oc3c(-c5ccccc5)cccc34)cc2)cc1] 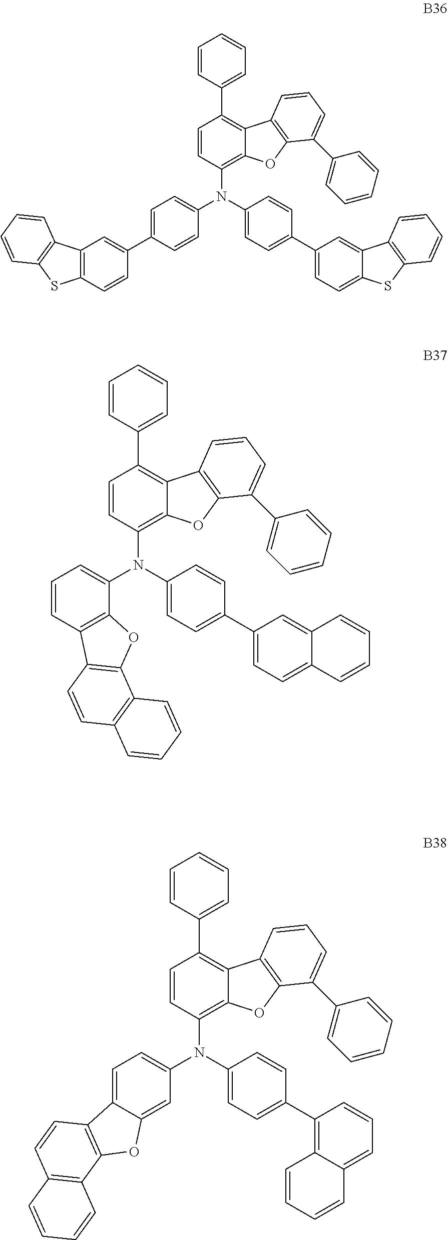 [SMILES: c1ccc(-c2cccc3c2oc2c(N(c4ccc(-c5ccc6sc7ccccc7c6c5)cc4)c4ccc(-c5ccc6sc7ccccc7c6c5)cc4)ccc(-c4ccccc4)c23)cc1 ; c1ccc(-c2cccc3c2oc2c(N(c4ccc(-c5ccc6ccccc6c5)cc4)c4cccc5c4oc4c6ccccc6ccc54)ccc(-c4ccccc4)c23)cc1 ; c1ccc(-c2cccc3c2oc2c(N(c4ccc(-c5cccc6ccccc56)cc4)c4ccc5c(c4)oc4c6ccccc6ccc54)ccc(-c4ccccc4)c23)cc1] 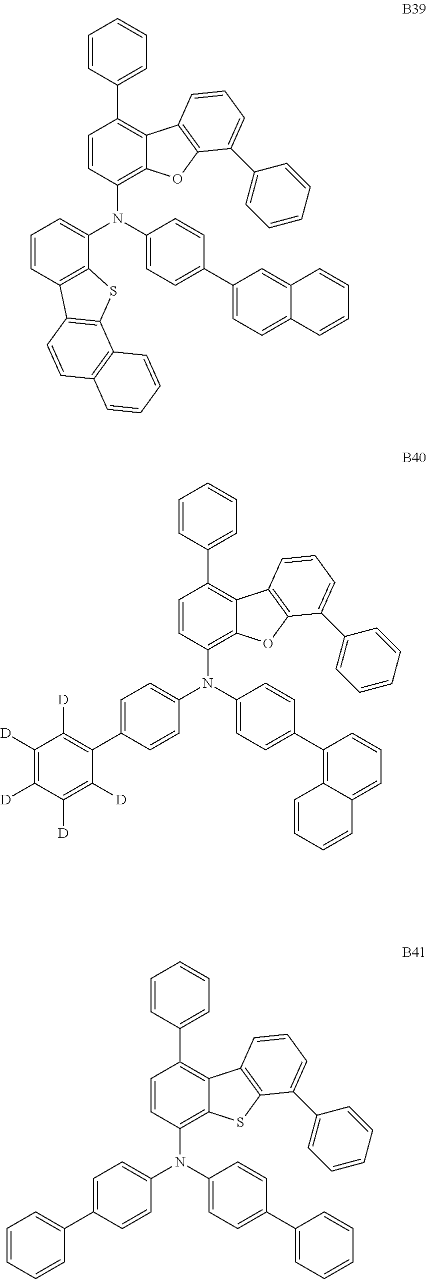 [SMILES: c1ccc(-c2cccc3c2oc2c(N(c4ccc(-c5ccc6ccccc6c5)cc4)c4cccc5c4sc4c6ccccc6ccc54)ccc(-c4ccccc4)c23)cc1 ; [2H]c1c([2H])c([2H])c(-c2ccc(N(c3ccc(-c4cccc5ccccc45)cc3)c3ccc(-c4ccccc4)c4c3oc3c(-c5ccccc5)cccc34)cc2)c([2H])c1[2H] ; c1ccc(-c2ccc(N(c3ccc(-c4ccccc4)cc3)c3ccc(-c4ccccc4)c4c3sc3c(-c5ccccc5)cccc34)cc2)cc1] 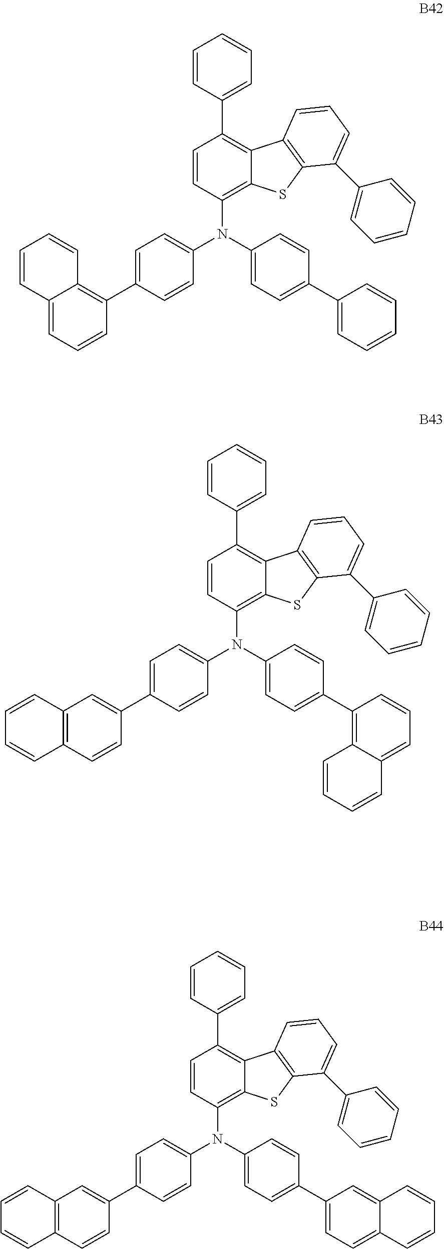 [SMILES: c1ccc(-c2ccc(N(c3ccc(-c4cccc5ccccc45)cc3)c3ccc(-c4ccccc4)c4c3sc3c(-c5ccccc5)cccc34)cc2)cc1 ; c1ccc(-c2cccc3c2sc2c(N(c4ccc(-c5ccc6ccccc6c5)cc4)c4ccc(-c5cccc6ccccc56)cc4)ccc(-c4ccccc4)c23)cc1 ; c1ccc(-c2cccc3c2sc2c(N(c4ccc(-c5ccc6ccccc6c5)cc4)c4ccc(-c5ccc6ccccc6c5)cc4)ccc(-c4ccccc4)c23)cc1] 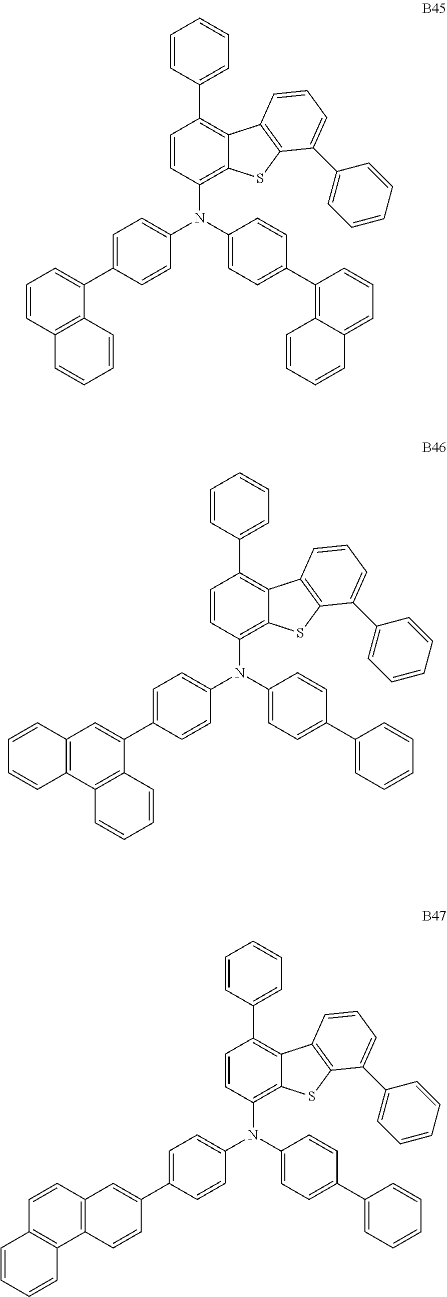 [SMILES: c1ccc(-c2cccc3c2sc2c(N(c4ccc(-c5cccc6ccccc56)cc4)c4ccc(-c5cccc6ccccc56)cc4)ccc(-c4ccccc4)c23)cc1 ; c1ccc(-c2ccc(N(c3ccc(-c4cc5ccccc5c5ccccc45)cc3)c3ccc(-c4ccccc4)c4c3sc3c(-c5ccccc5)cccc34)cc2)cc1 ; c1ccc(-c2ccc(N(c3ccc(-c4ccc5c(ccc6ccccc65)c4)cc3)c3ccc(-c4ccccc4)c4c3sc3c(-c5ccccc5)cccc34)cc2)cc1] 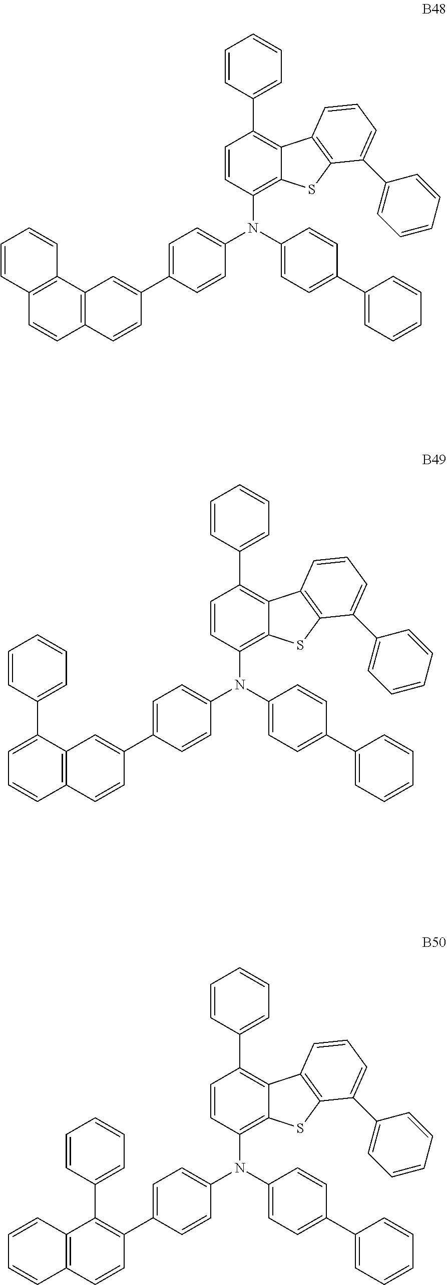 [SMILES: c1ccc(-c2ccc(N(c3ccc(-c4ccc5ccc6ccccc6c5c4)cc3)c3ccc(-c4ccccc4)c4c3sc3c(-c5ccccc5)cccc34)cc2)cc1 ; c1ccc(-c2ccc(N(c3ccc(-c4ccc5cccc(-c6ccccc6)c5c4)cc3)c3ccc(-c4ccccc4)c4c3sc3c(-c5ccccc5)cccc34)cc2)cc1 ; c1ccc(-c2ccc(N(c3ccc(-c4ccc5ccccc5c4-c4ccccc4)cc3)c3ccc(-c4ccccc4)c4c3sc3c(-c5ccccc5)cccc34)cc2)cc1] 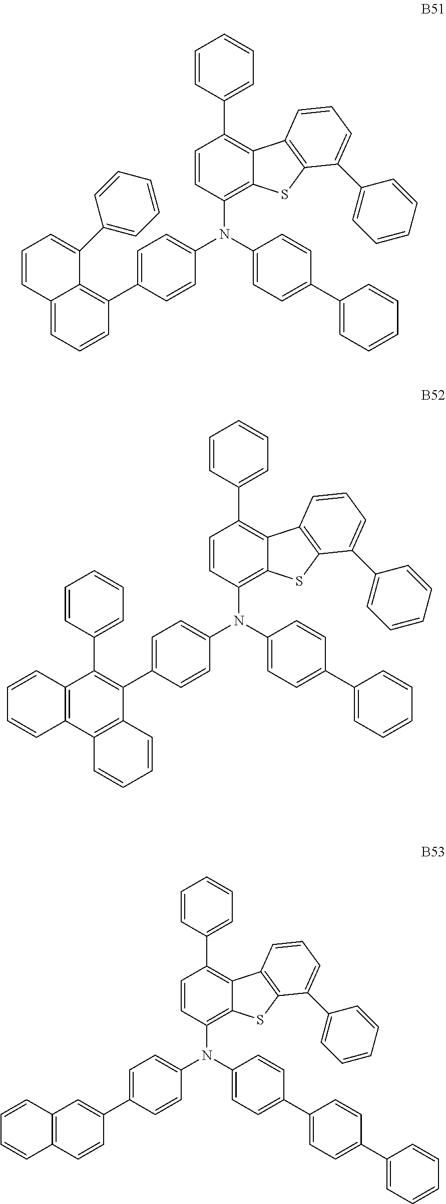 [SMILES: c1ccc(-c2ccc(N(c3ccc(-c4cccc5cccc(-c6ccccc6)c45)cc3)c3ccc(-c4ccccc4)c4c3sc3c(-c5ccccc5)cccc34)cc2)cc1 ; c1ccc(-c2ccc(N(c3ccc(-c4c(-c5ccccc5)c5ccccc5c5ccccc45)cc3)c3ccc(-c4ccccc4)c4c3sc3c(-c5ccccc5)cccc34)cc2)cc1 ; c1ccc(-c2ccc(-c3ccc(N(c4ccc(-c5ccc6ccccc6c5)cc4)c4ccc(-c5ccccc5)c5c4sc4c(-c6ccccc6)cccc45)cc3)cc2)cc1] 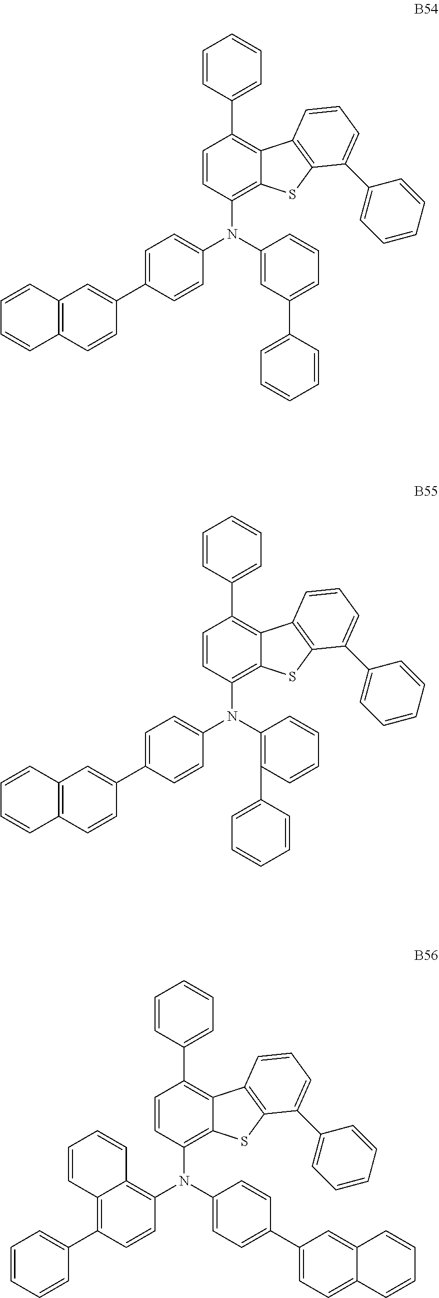 [SMILES: c1ccc(-c2cccc(N(c3ccc(-c4ccc5ccccc5c4)cc3)c3ccc(-c4ccccc4)c4c3sc3c(-c5ccccc5)cccc34)c2)cc1 ; c1ccc(-c2ccccc2N(c2ccc(-c3ccc4ccccc4c3)cc2)c2ccc(-c3ccccc3)c3c2sc2c(-c4ccccc4)cccc23)cc1 ; c1ccc(-c2ccc(N(c3ccc(-c4ccc5ccccc5c4)cc3)c3ccc(-c4ccccc4)c4c3sc3c(-c5ccccc5)cccc34)c3ccccc23)cc1] 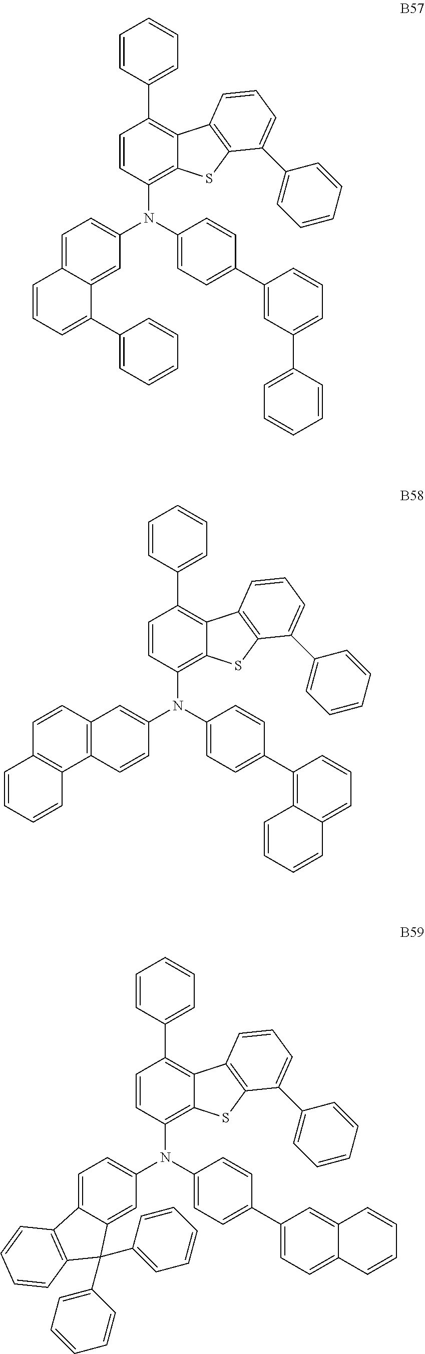 [SMILES: c1ccc(-c2cccc(-c3ccc(N(c4ccc5cccc(-c6ccccc6)c5c4)c4ccc(-c5ccccc5)c5c4sc4c(-c6ccccc6)cccc45)cc3)c2)cc1 ; c1ccc(-c2cccc3c2sc2c(N(c4ccc(-c5cccc6ccccc56)cc4)c4ccc5c(ccc6ccccc65)c4)ccc(-c4ccccc4)c23)cc1 ; c1ccc(-c2cccc3c2sc2c(N(c4ccc(-c5ccc6ccccc6c5)cc4)c4ccc5c(c4)C(c4ccccc4)(c4ccccc4)c4ccccc4-5)ccc(-c4ccccc4)c23)cc1] 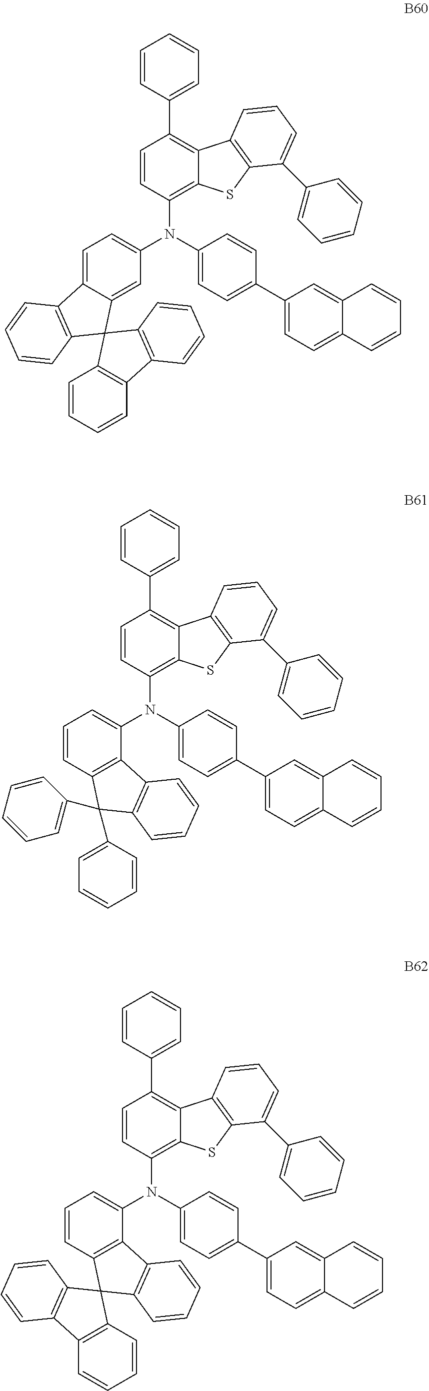 [SMILES: c1ccc(-c2cccc3c2sc2c(N(c4ccc(-c5ccc6ccccc6c5)cc4)c4ccc5c(c4)C4(c6ccccc6-c6ccccc64)c4ccccc4-5)ccc(-c4ccccc4)c23)cc1 ; c1ccc(-c2cccc3c2sc2c(N(c4ccc(-c5ccc6ccccc6c5)cc4)c4cccc5c4-c4ccccc4C5(c4ccccc4)c4ccccc4)ccc(-c4ccccc4)c23)cc1 ; c1ccc(-c2cccc3c2sc2c(N(c4ccc(-c5ccc6ccccc6c5)cc4)c4cccc5c4-c4ccccc4C54c5ccccc5-c5ccccc54)ccc(-c4ccccc4)c23)cc1] 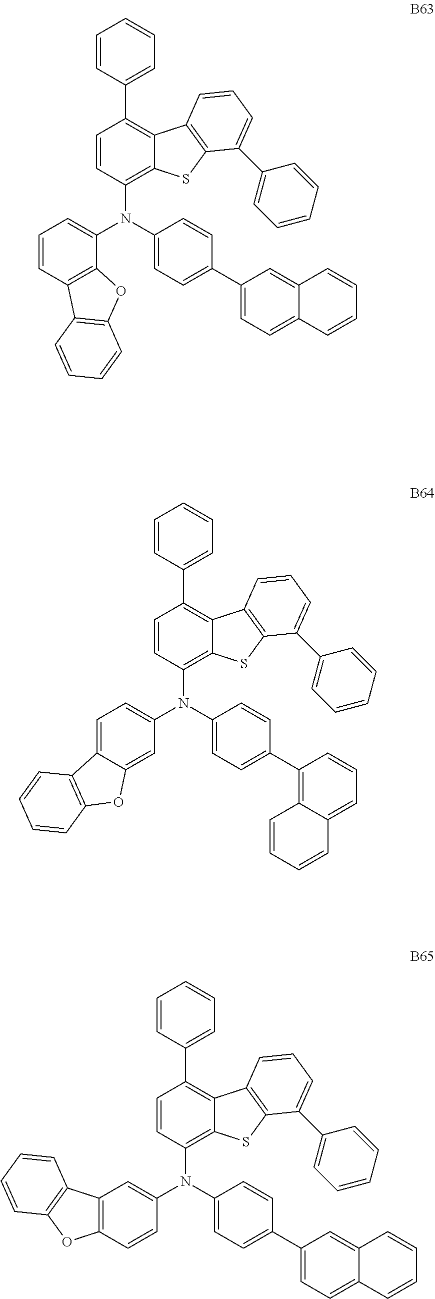 [SMILES: c1ccc(-c2cccc3c2sc2c(N(c4ccc(-c5ccc6ccccc6c5)cc4)c4cccc5c4oc4ccccc45)ccc(-c4ccccc4)c23)cc1 ; c1ccc(-c2cccc3c2sc2c(N(c4ccc(-c5cccc6ccccc56)cc4)c4ccc5c(c4)oc4ccccc45)ccc(-c4ccccc4)c23)cc1 ; c1ccc(-c2cccc3c2sc2c(N(c4ccc(-c5ccc6ccccc6c5)cc4)c4ccc5oc6ccccc6c5c4)ccc(-c4ccccc4)c23)cc1] 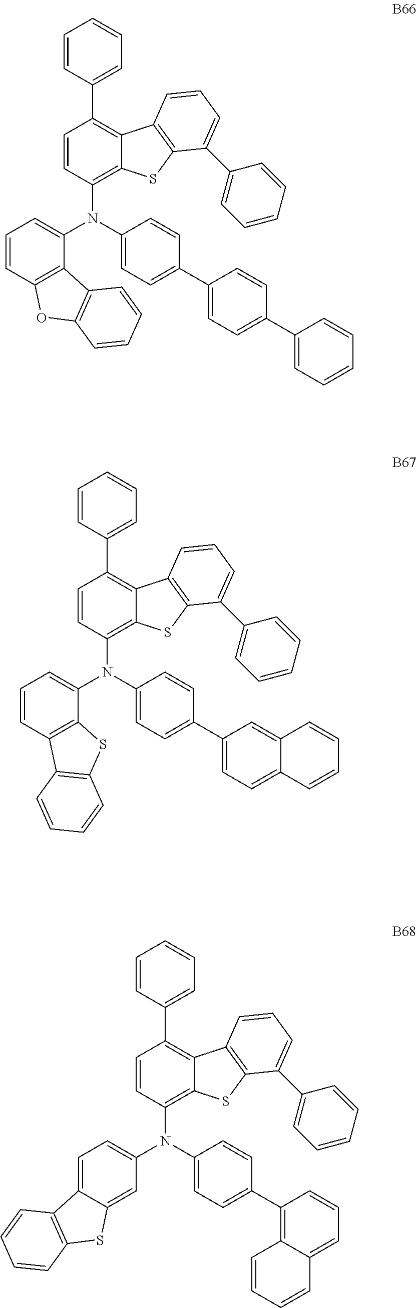 [SMILES: c1ccc(-c2ccc(-c3ccc(N(c4ccc(-c5ccccc5)c5c4sc4c(-c6ccccc6)cccc45)c4cccc5oc6ccccc6c45)cc3)cc2)cc1 ; c1ccc(-c2cccc3c2sc2c(N(c4ccc(-c5ccc6ccccc6c5)cc4)c4cccc5c4sc4ccccc45)ccc(-c4ccccc4)c23)cc1 ; c1ccc(-c2cccc3c2sc2c(N(c4ccc(-c5cccc6ccccc56)cc4)c4ccc5c(c4)sc4ccccc45)ccc(-c4ccccc4)c23)cc1] 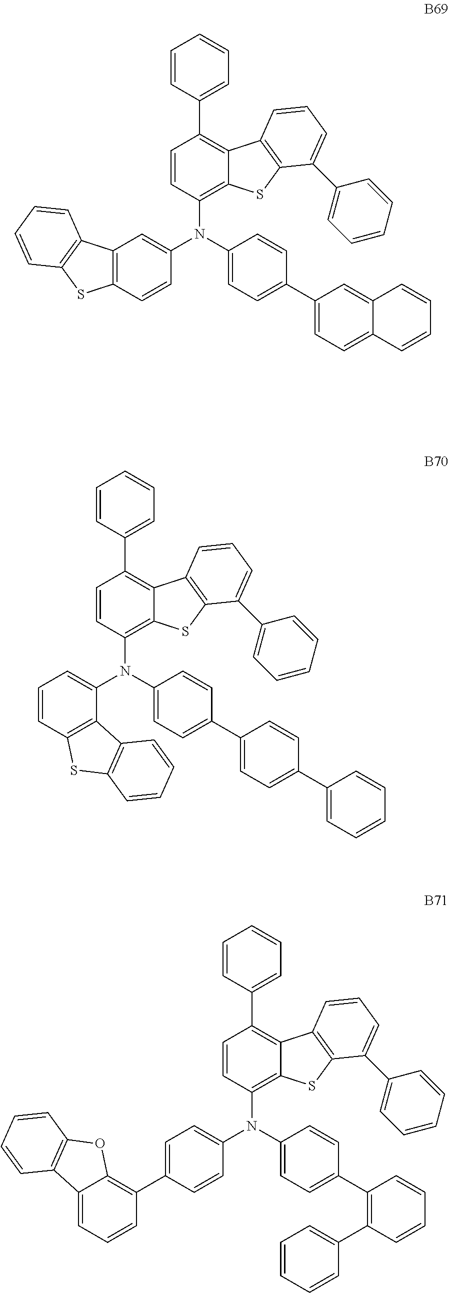 [SMILES: c1ccc(-c2cccc3c2sc2c(N(c4ccc(-c5ccc6ccccc6c5)cc4)c4ccc5sc6ccccc6c5c4)ccc(-c4ccccc4)c23)cc1 ; c1ccc(-c2ccc(-c3ccc(N(c4ccc(-c5ccccc5)c5c4sc4c(-c6ccccc6)cccc45)c4cccc5sc6ccccc6c45)cc3)cc2)cc1 ; c1ccc(-c2ccccc2-c2ccc(N(c3ccc(-c4cccc5c4oc4ccccc45)cc3)c3ccc(-c4ccccc4)c4c3sc3c(-c5ccccc5)cccc34)cc2)cc1] 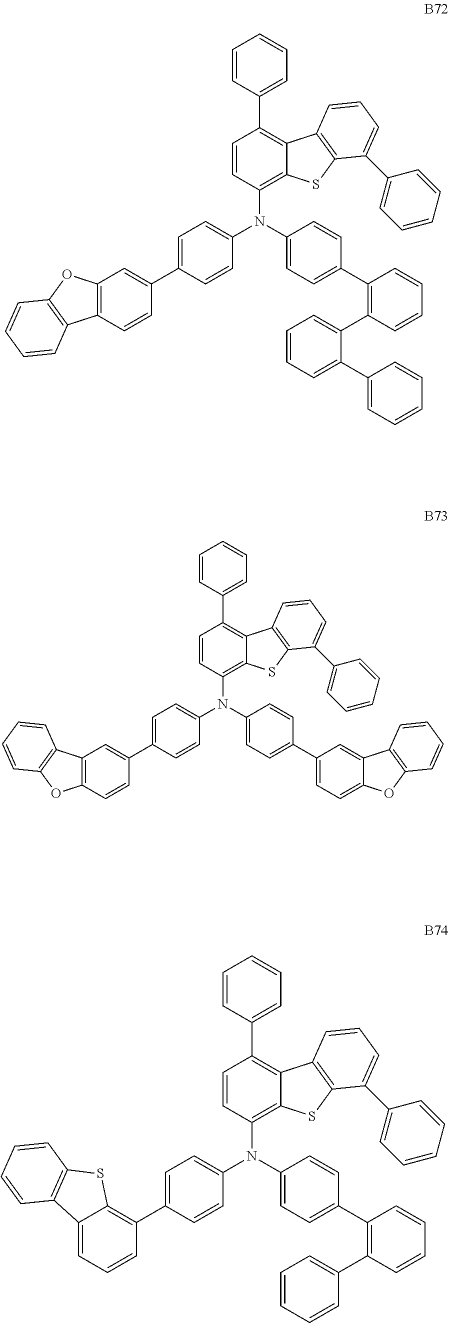 [SMILES: c1ccc(-c2ccccc2-c2ccccc2-c2ccc(N(c3ccc(-c4ccc5c(c4)oc4ccccc45)cc3)c3ccc(-c4ccccc4)c4c3sc3c(-c5ccccc5)cccc34)cc2)cc1 ; c1ccc(-c2cccc3c2sc2c(N(c4ccc(-c5ccc6oc7ccccc7c6c5)cc4)c4ccc(-c5ccc6oc7ccccc7c6c5)cc4)ccc(-c4ccccc4)c23)cc1 ; c1ccc(-c2ccccc2-c2ccc(N(c3ccc(-c4cccc5c4sc4ccccc45)cc3)c3ccc(-c4ccccc4)c4c3sc3c(-c5ccccc5)cccc34)cc2)cc1] 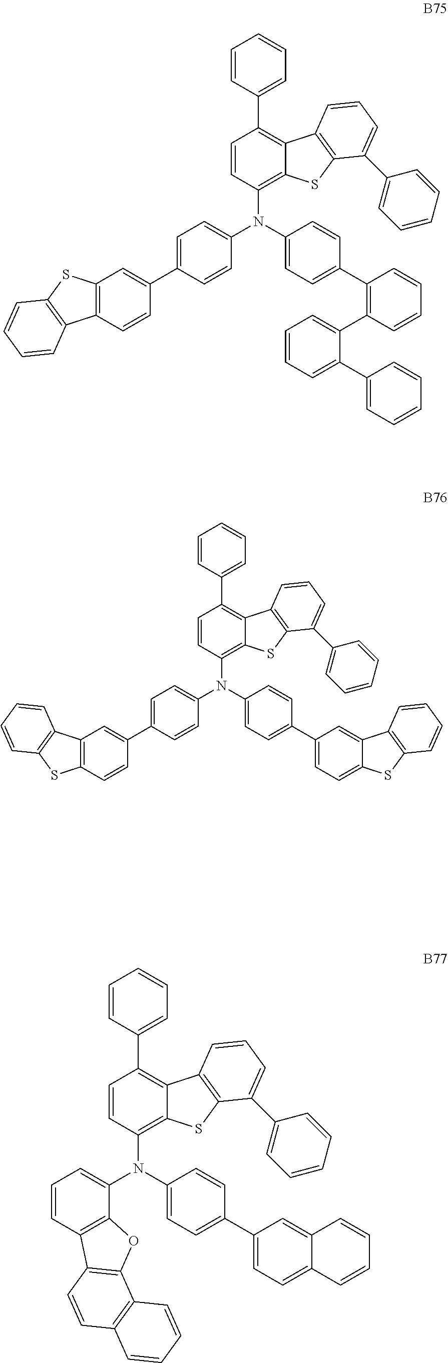 [SMILES: c1ccc(-c2ccccc2-c2ccccc2-c2ccc(N(c3ccc(-c4ccc5c(c4)sc4ccccc45)cc3)c3ccc(-c4ccccc4)c4c3sc3c(-c5ccccc5)cccc34)cc2)cc1 ; c1ccc(-c2cccc3c2sc2c(N(c4ccc(-c5ccc6sc7ccccc7c6c5)cc4)c4ccc(-c5ccc6sc7ccccc7c6c5)cc4)ccc(-c4ccccc4)c23)cc1 ; c1ccc(-c2cccc3c2sc2c(N(c4ccc(-c5ccc6ccccc6c5)cc4)c4cccc5c4oc4c6ccccc6ccc54)ccc(-c4ccccc4)c23)cc1] 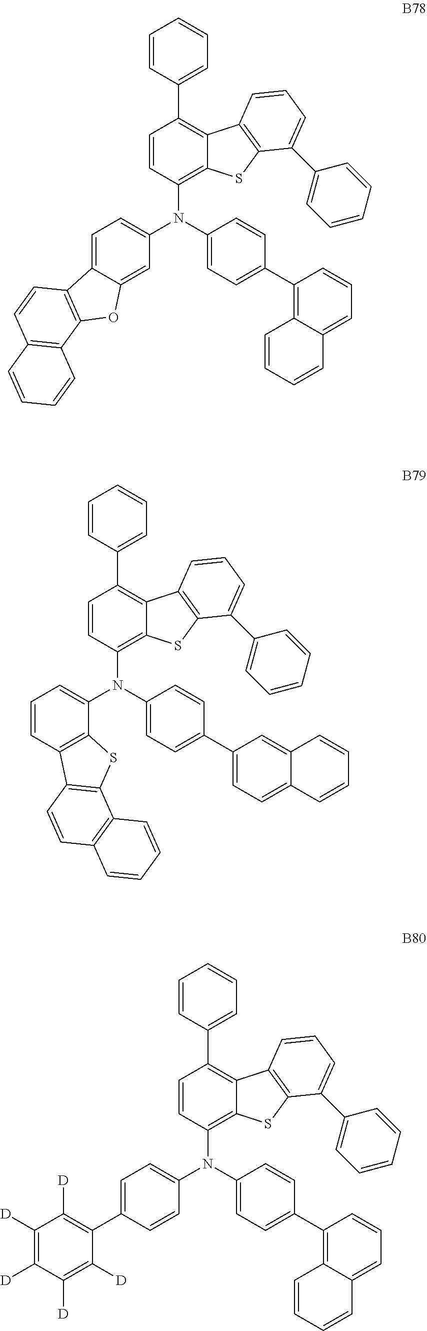 [SMILES: c1ccc(-c2cccc3c2sc2c(N(c4ccc(-c5cccc6ccccc56)cc4)c4ccc5c(c4)oc4c6ccccc6ccc54)ccc(-c4ccccc4)c23)cc1 ; c1ccc(-c2cccc3c2sc2c(N(c4ccc(-c5ccc6ccccc6c5)cc4)c4cccc5c4sc4c6ccccc6ccc54)ccc(-c4ccccc4)c23)cc1 ; [2H]c1c([2H])c([2H])c(-c2ccc(N(c3ccc(-c4cccc5ccccc45)cc3)c3ccc(-c4ccccc4)c4c3sc3c(-c5ccccc5)cccc34)cc2)c([2H])c1[2H]] 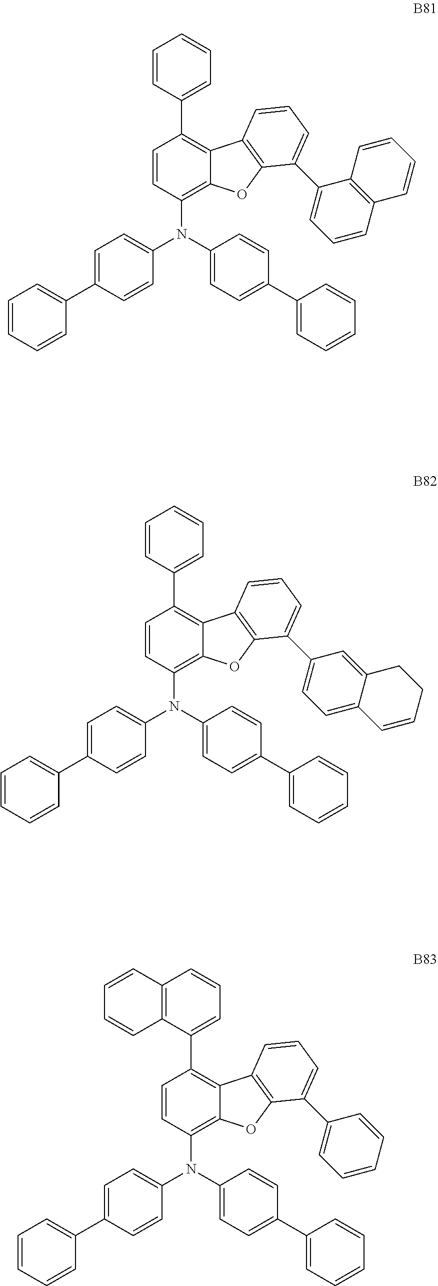 [SMILES: c1ccc(-c2ccc(N(c3ccc(-c4ccccc4)cc3)c3ccc(-c4ccccc4)c4c3oc3c(-c5cccc6ccccc56)cccc34)cc2)cc1 ; C1=Cc2ccc(-c3cccc4c3oc3c(N(c5ccc(-c6ccccc6)cc5)c5ccc(-c6ccccc6)cc5)ccc(-c5ccccc5)c34)cc2CC1 ; c1ccc(-c2ccc(N(c3ccc(-c4ccccc4)cc3)c3ccc(-c4cccc5ccccc45)c4c3oc3c(-c5ccccc5)cccc34)cc2)cc1] 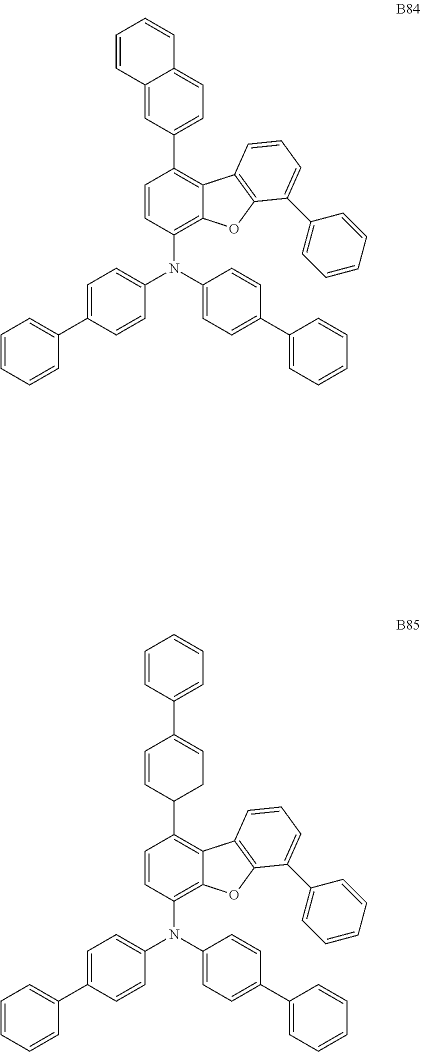 [SMILES: c1ccc(-c2ccc(N(c3ccc(-c4ccccc4)cc3)c3ccc(-c4ccc5ccccc5c4)c4c3oc3c(-c5ccccc5)cccc34)cc2)cc1 ; C1=CC(c2ccc(N(c3ccc(-c4ccccc4)cc3)c3ccc(-c4ccccc4)cc3)c3oc4c(-c5ccccc5)cccc4c23)CC=C1c1ccccc1] 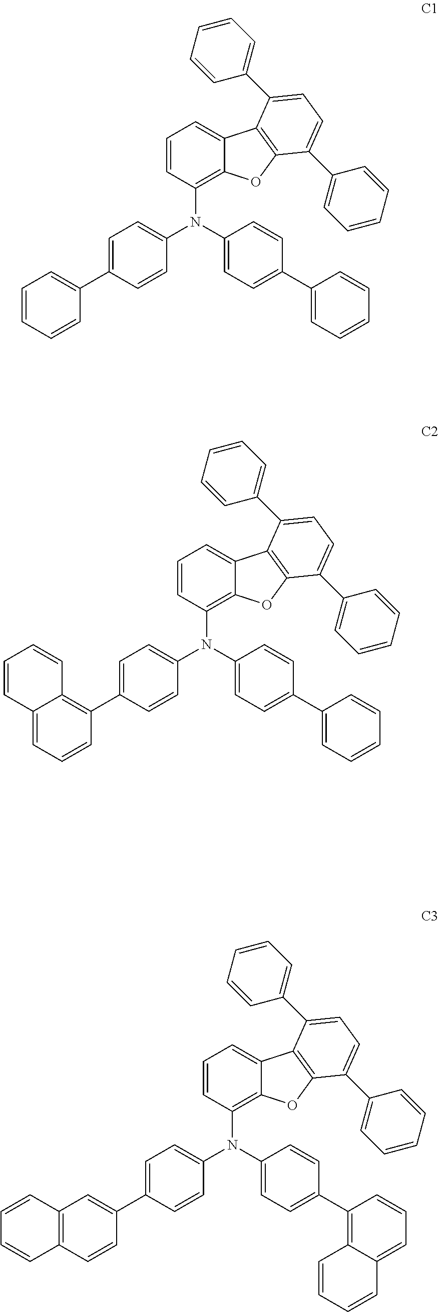 [SMILES: c1ccc(-c2ccc(N(c3ccc(-c4ccccc4)cc3)c3cccc4c3oc3c(-c5ccccc5)ccc(-c5ccccc5)c34)cc2)cc1 ; c1ccc(-c2ccc(N(c3ccc(-c4cccc5ccccc45)cc3)c3cccc4c3oc3c(-c5ccccc5)ccc(-c5ccccc5)c34)cc2)cc1 ; c1ccc(-c2ccc(-c3ccccc3)c3c2oc2c(N(c4ccc(-c5ccc6ccccc6c5)cc4)c4ccc(-c5cccc6ccccc56)cc4)cccc23)cc1] 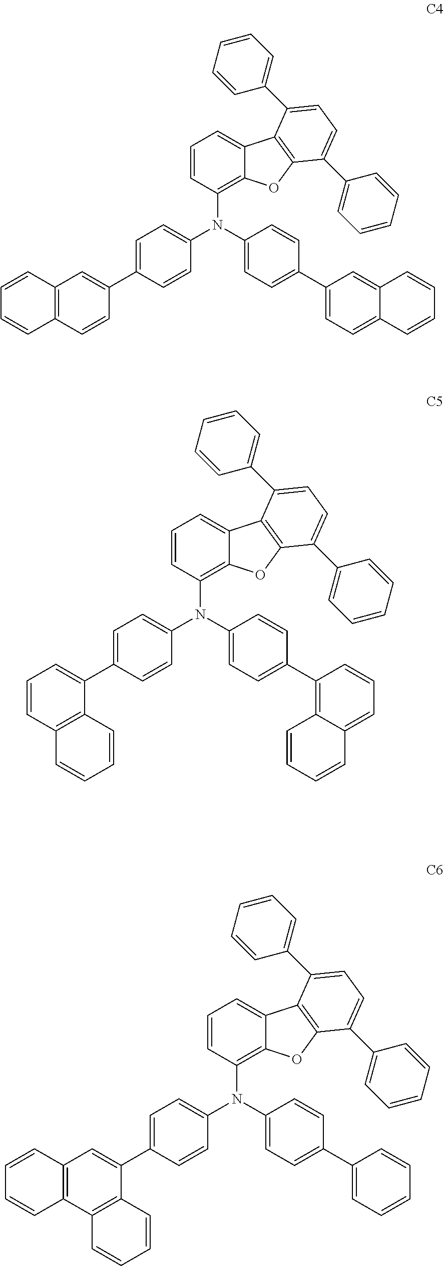 [SMILES: c1ccc(-c2ccc(-c3ccccc3)c3c2oc2c(N(c4ccc(-c5ccc6ccccc6c5)cc4)c4ccc(-c5ccc6ccccc6c5)cc4)cccc23)cc1 ; c1ccc(-c2ccc(-c3ccccc3)c3c2oc2c(N(c4ccc(-c5cccc6ccccc56)cc4)c4ccc(-c5cccc6ccccc56)cc4)cccc23)cc1 ; c1ccc(-c2ccc(N(c3ccc(-c4cc5ccccc5c5ccccc45)cc3)c3cccc4c3oc3c(-c5ccccc5)ccc(-c5ccccc5)c34)cc2)cc1] 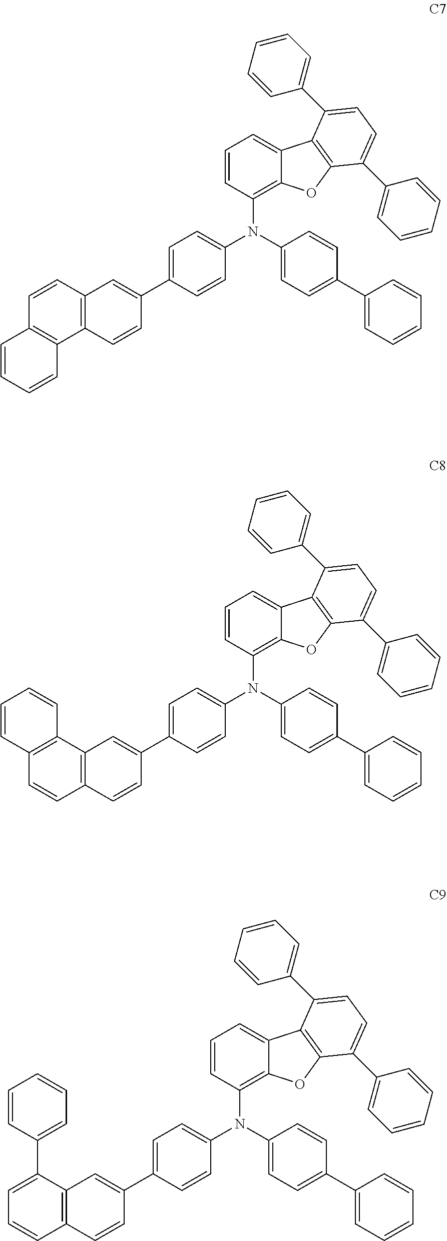 [SMILES: c1ccc(-c2ccc(N(c3ccc(-c4ccc5c(ccc6ccccc65)c4)cc3)c3cccc4c3oc3c(-c5ccccc5)ccc(-c5ccccc5)c34)cc2)cc1 ; c1ccc(-c2ccc(N(c3ccc(-c4ccc5ccc6ccccc6c5c4)cc3)c3cccc4c3oc3c(-c5ccccc5)ccc(-c5ccccc5)c34)cc2)cc1 ; c1ccc(-c2ccc(N(c3ccc(-c4ccc5cccc(-c6ccccc6)c5c4)cc3)c3cccc4c3oc3c(-c5ccccc5)ccc(-c5ccccc5)c34)cc2)cc1] 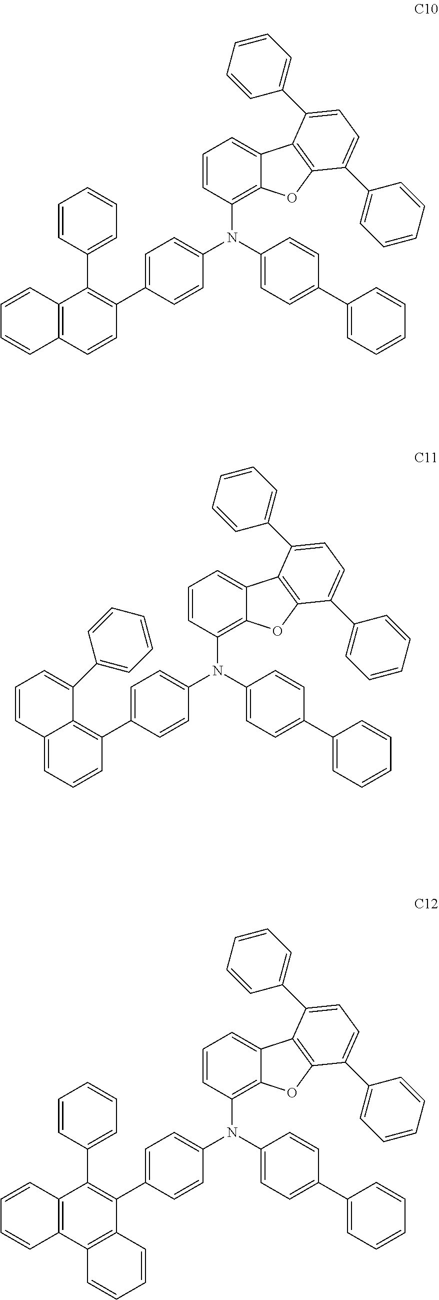 [SMILES: c1ccc(-c2ccc(N(c3ccc(-c4ccc5ccccc5c4-c4ccccc4)cc3)c3cccc4c3oc3c(-c5ccccc5)ccc(-c5ccccc5)c34)cc2)cc1 ; c1ccc(-c2ccc(N(c3ccc(-c4cccc5cccc(-c6ccccc6)c45)cc3)c3cccc4c3oc3c(-c5ccccc5)ccc(-c5ccccc5)c34)cc2)cc1 ; c1ccc(-c2ccc(N(c3ccc(-c4c(-c5ccccc5)c5ccccc5c5ccccc45)cc3)c3cccc4c3oc3c(-c5ccccc5)ccc(-c5ccccc5)c34)cc2)cc1] 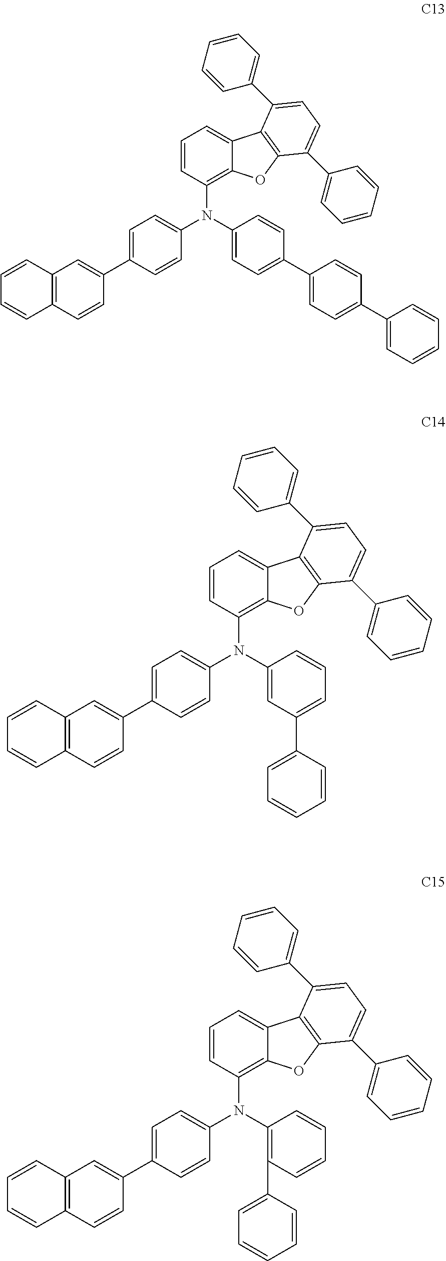 [SMILES: c1ccc(-c2ccc(-c3ccc(N(c4ccc(-c5ccc6ccccc6c5)cc4)c4cccc5c4oc4c(-c6ccccc6)ccc(-c6ccccc6)c45)cc3)cc2)cc1 ; c1ccc(-c2cccc(N(c3ccc(-c4ccc5ccccc5c4)cc3)c3cccc4c3oc3c(-c5ccccc5)ccc(-c5ccccc5)c34)c2)cc1 ; c1ccc(-c2ccccc2N(c2ccc(-c3ccc4ccccc4c3)cc2)c2cccc3c2oc2c(-c4ccccc4)ccc(-c4ccccc4)c23)cc1] 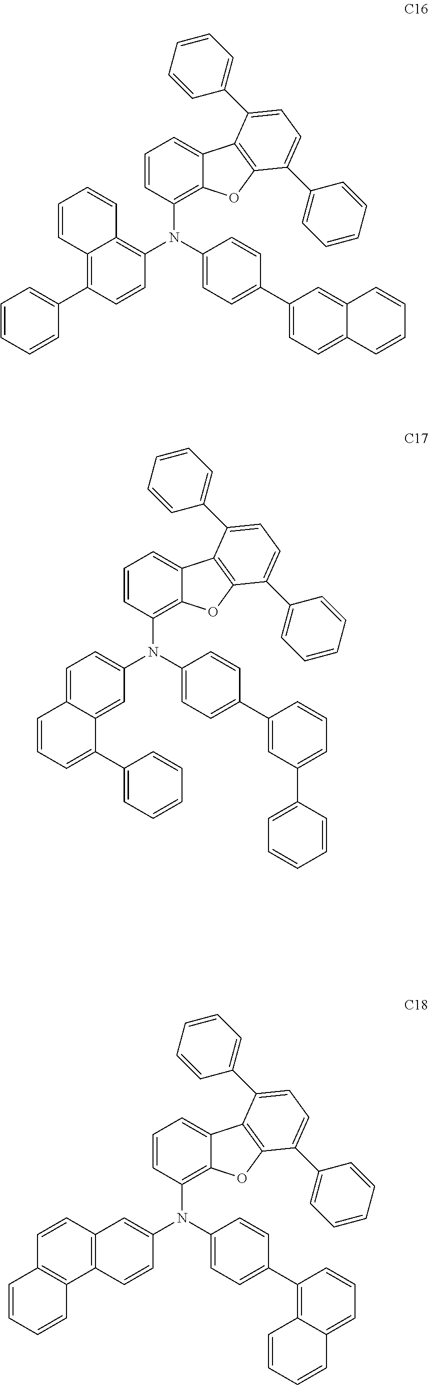 [SMILES: c1ccc(-c2ccc(N(c3ccc(-c4ccc5ccccc5c4)cc3)c3cccc4c3oc3c(-c5ccccc5)ccc(-c5ccccc5)c34)c3ccccc23)cc1 ; c1ccc(-c2cccc(-c3ccc(N(c4ccc5cccc(-c6ccccc6)c5c4)c4cccc5c4oc4c(-c6ccccc6)ccc(-c6ccccc6)c45)cc3)c2)cc1 ; c1ccc(-c2ccc(-c3ccccc3)c3c2oc2c(N(c4ccc(-c5cccc6ccccc56)cc4)c4ccc5c(ccc6ccccc65)c4)cccc23)cc1] 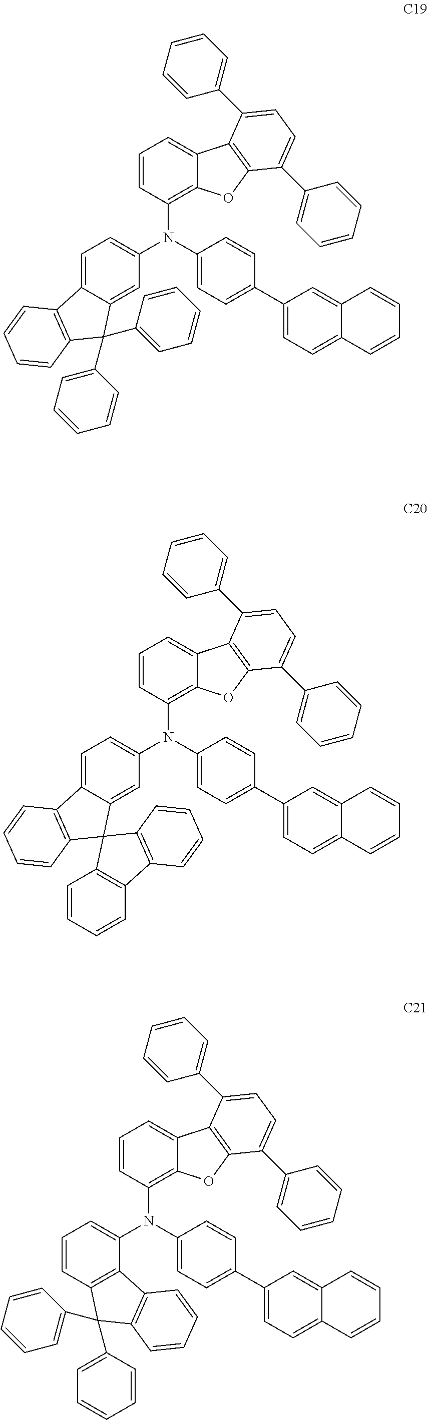 [SMILES: c1ccc(-c2ccc(-c3ccccc3)c3c2oc2c(N(c4ccc(-c5ccc6ccccc6c5)cc4)c4ccc5c(c4)C(c4ccccc4)(c4ccccc4)c4ccccc4-5)cccc23)cc1 ; c1ccc(-c2ccc(-c3ccccc3)c3c2oc2c(N(c4ccc(-c5ccc6ccccc6c5)cc4)c4ccc5c(c4)C4(c6ccccc6-c6ccccc64)c4ccccc4-5)cccc23)cc1 ; c1ccc(-c2ccc(-c3ccccc3)c3c2oc2c(N(c4ccc(-c5ccc6ccccc6c5)cc4)c4cccc5c4-c4ccccc4C5(c4ccccc4)c4ccccc4)cccc23)cc1] 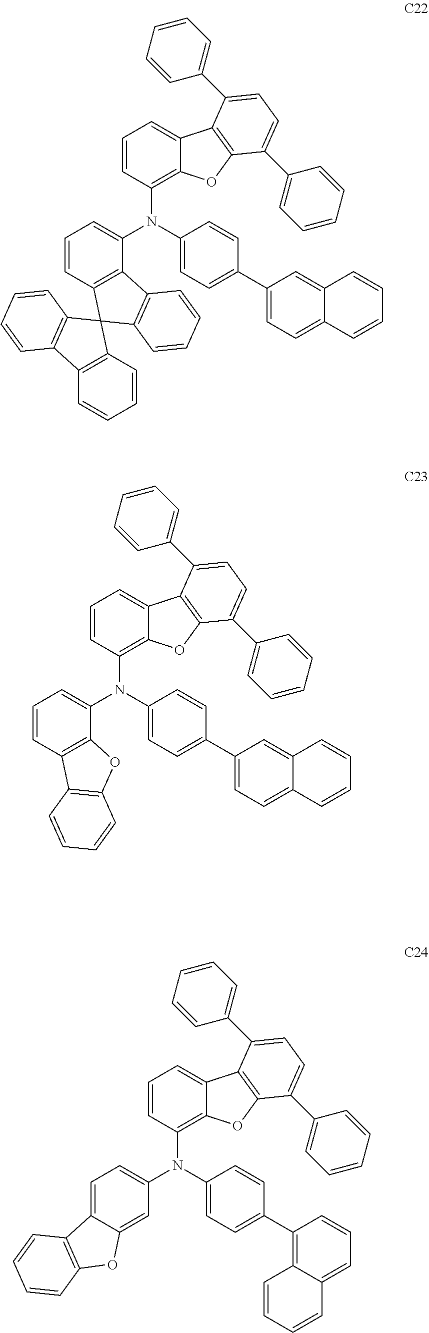 [SMILES: c1ccc(-c2ccc(-c3ccccc3)c3c2oc2c(N(c4ccc(-c5ccc6ccccc6c5)cc4)c4cccc5c4-c4ccccc4C54c5ccccc5-c5ccccc54)cccc23)cc1 ; c1ccc(-c2ccc(-c3ccccc3)c3c2oc2c(N(c4ccc(-c5ccc6ccccc6c5)cc4)c4cccc5c4oc4ccccc45)cccc23)cc1 ; c1ccc(-c2ccc(-c3ccccc3)c3c2oc2c(N(c4ccc(-c5cccc6ccccc56)cc4)c4ccc5c(c4)oc4ccccc45)cccc23)cc1] 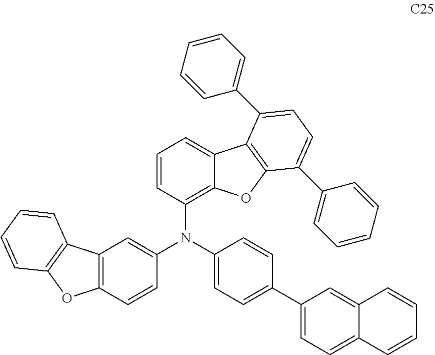 [SMILES: c1ccc(-c2ccc(-c3ccccc3)c3c2oc2c(N(c4ccc(-c5ccc6ccccc6c5)cc4)c4ccc5oc6ccccc6c5c4)cccc23)cc1] 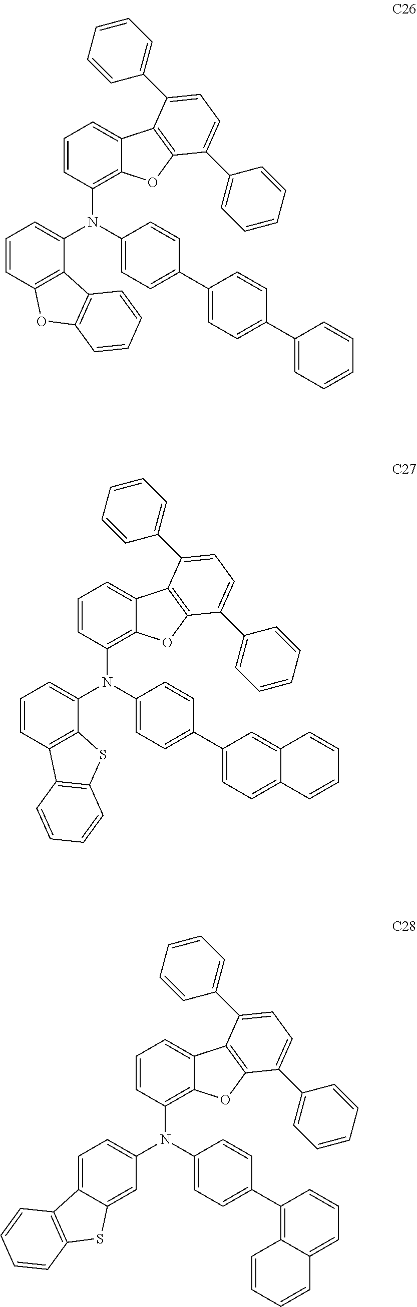 [SMILES: c1ccc(-c2ccc(-c3ccc(N(c4cccc5c4oc4c(-c6ccccc6)ccc(-c6ccccc6)c45)c4cccc5oc6ccccc6c45)cc3)cc2)cc1 ; c1ccc(-c2ccc(-c3ccccc3)c3c2oc2c(N(c4ccc(-c5ccc6ccccc6c5)cc4)c4cccc5c4sc4ccccc45)cccc23)cc1 ; c1ccc(-c2ccc(-c3ccccc3)c3c2oc2c(N(c4ccc(-c5cccc6ccccc56)cc4)c4ccc5c(c4)sc4ccccc45)cccc23)cc1] 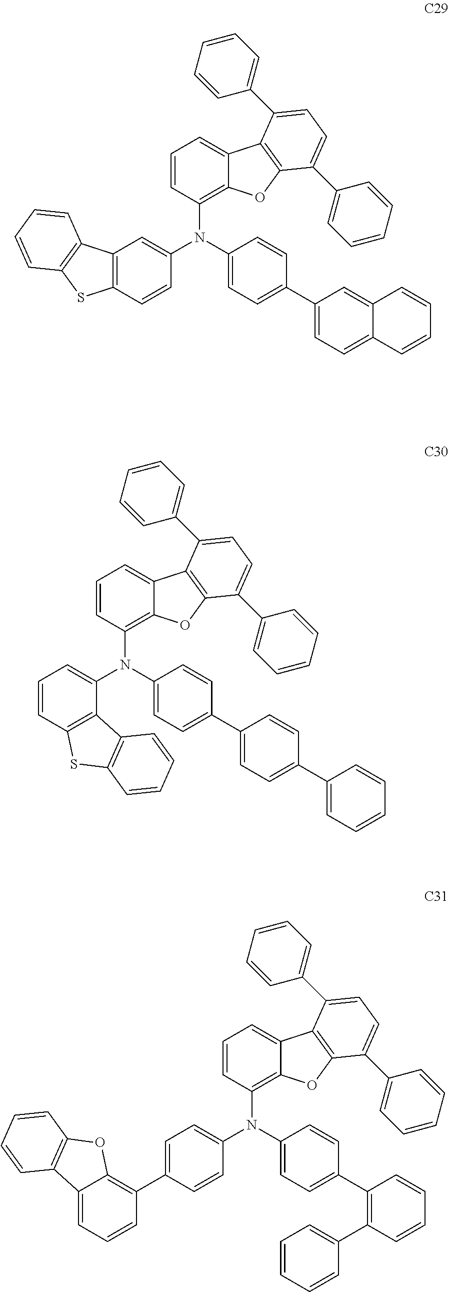 [SMILES: c1ccc(-c2ccc(-c3ccccc3)c3c2oc2c(N(c4ccc(-c5ccc6ccccc6c5)cc4)c4ccc5sc6ccccc6c5c4)cccc23)cc1 ; c1ccc(-c2ccc(-c3ccc(N(c4cccc5c4oc4c(-c6ccccc6)ccc(-c6ccccc6)c45)c4cccc5sc6ccccc6c45)cc3)cc2)cc1 ; c1ccc(-c2ccccc2-c2ccc(N(c3ccc(-c4cccc5c4oc4ccccc45)cc3)c3cccc4c3oc3c(-c5ccccc5)ccc(-c5ccccc5)c34)cc2)cc1] 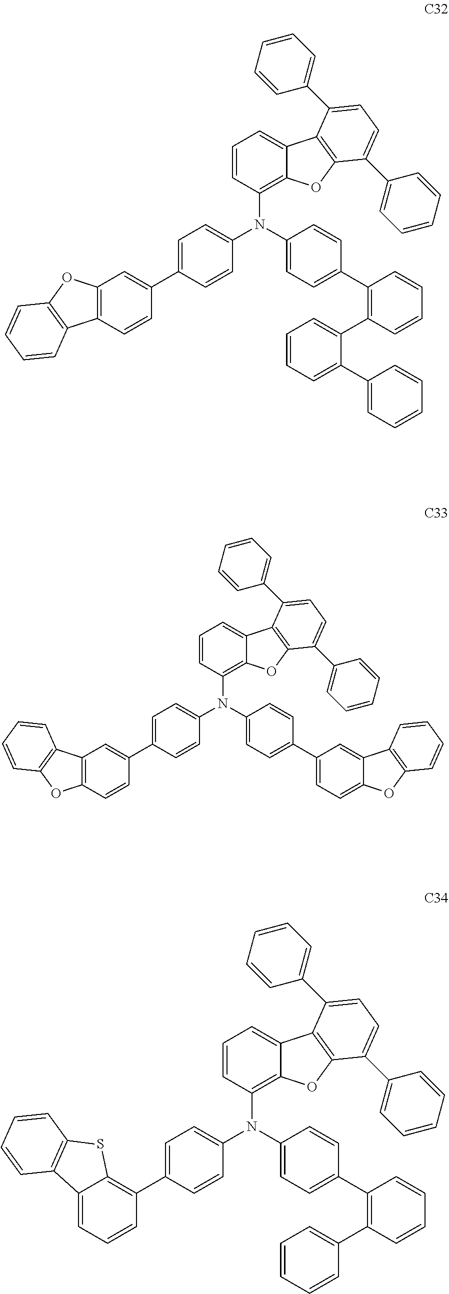 [SMILES: c1ccc(-c2ccccc2-c2ccccc2-c2ccc(N(c3ccc(-c4ccc5c(c4)oc4ccccc45)cc3)c3cccc4c3oc3c(-c5ccccc5)ccc(-c5ccccc5)c34)cc2)cc1 ; c1ccc(-c2ccc(-c3ccccc3)c3c2oc2c(N(c4ccc(-c5ccc6oc7ccccc7c6c5)cc4)c4ccc(-c5ccc6oc7ccccc7c6c5)cc4)cccc23)cc1 ; c1ccc(-c2ccccc2-c2ccc(N(c3ccc(-c4cccc5c4sc4ccccc45)cc3)c3cccc4c3oc3c(-c5ccccc5)ccc(-c5ccccc5)c34)cc2)cc1] 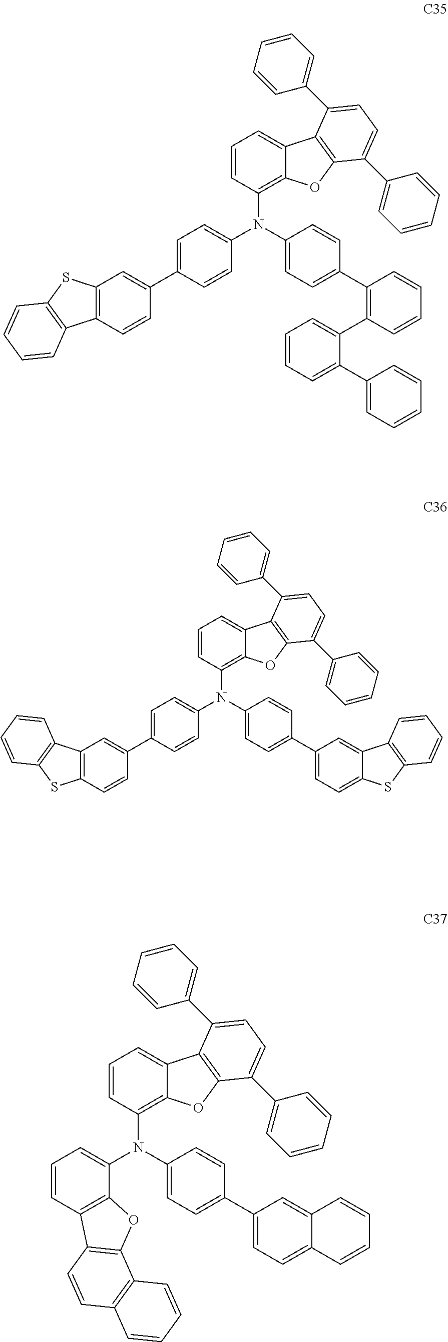 [SMILES: c1ccc(-c2ccccc2-c2ccccc2-c2ccc(N(c3ccc(-c4ccc5c(c4)sc4ccccc45)cc3)c3cccc4c3oc3c(-c5ccccc5)ccc(-c5ccccc5)c34)cc2)cc1 ; c1ccc(-c2ccc(-c3ccccc3)c3c2oc2c(N(c4ccc(-c5ccc6sc7ccccc7c6c5)cc4)c4ccc(-c5ccc6sc7ccccc7c6c5)cc4)cccc23)cc1 ; c1ccc(-c2ccc(-c3ccccc3)c3c2oc2c(N(c4ccc(-c5ccc6ccccc6c5)cc4)c4cccc5c4oc4c6ccccc6ccc54)cccc23)cc1] 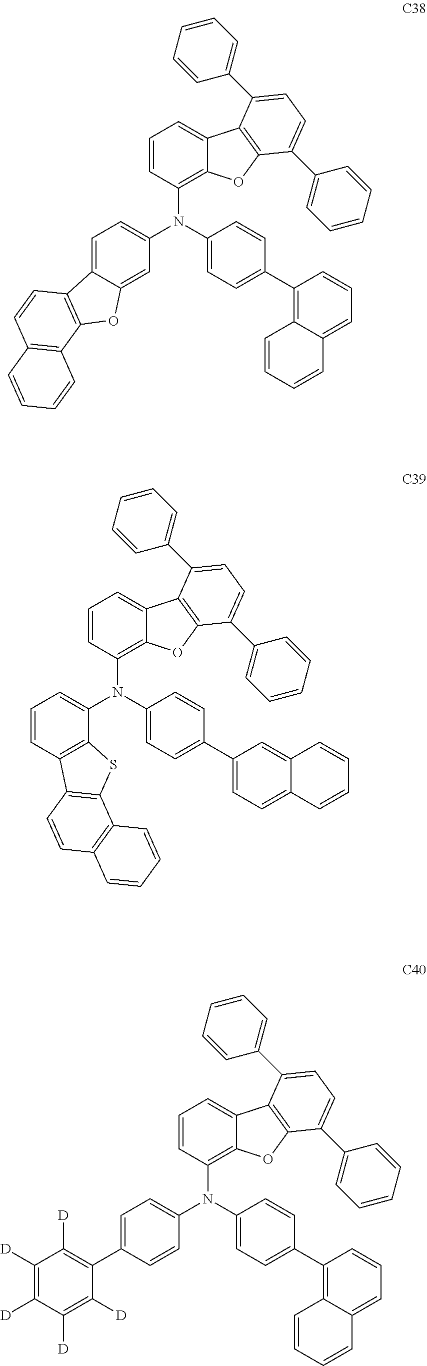 [SMILES: c1ccc(-c2ccc(-c3ccccc3)c3c2oc2c(N(c4ccc(-c5cccc6ccccc56)cc4)c4ccc5c(c4)oc4c6ccccc6ccc54)cccc23)cc1 ; c1ccc(-c2ccc(-c3ccccc3)c3c2oc2c(N(c4ccc(-c5ccc6ccccc6c5)cc4)c4cccc5c4sc4c6ccccc6ccc54)cccc23)cc1 ; [2H]c1c([2H])c([2H])c(-c2ccc(N(c3ccc(-c4cccc5ccccc45)cc3)c3cccc4c3oc3c(-c5ccccc5)ccc(-c5ccccc5)c34)cc2)c([2H])c1[2H]] 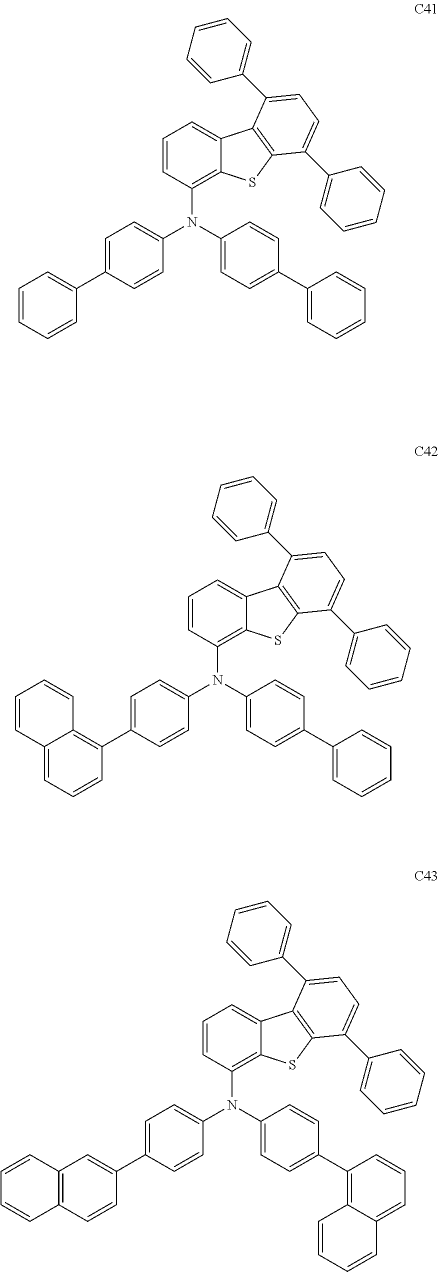 [SMILES: c1ccc(-c2ccc(N(c3ccc(-c4ccccc4)cc3)c3cccc4c3sc3c(-c5ccccc5)ccc(-c5ccccc5)c34)cc2)cc1 ; c1ccc(-c2ccc(N(c3ccc(-c4cccc5ccccc45)cc3)c3cccc4c3sc3c(-c5ccccc5)ccc(-c5ccccc5)c34)cc2)cc1 ; c1ccc(-c2ccc(-c3ccccc3)c3c2sc2c(N(c4ccc(-c5ccc6ccccc6c5)cc4)c4ccc(-c5cccc6ccccc56)cc4)cccc23)cc1] 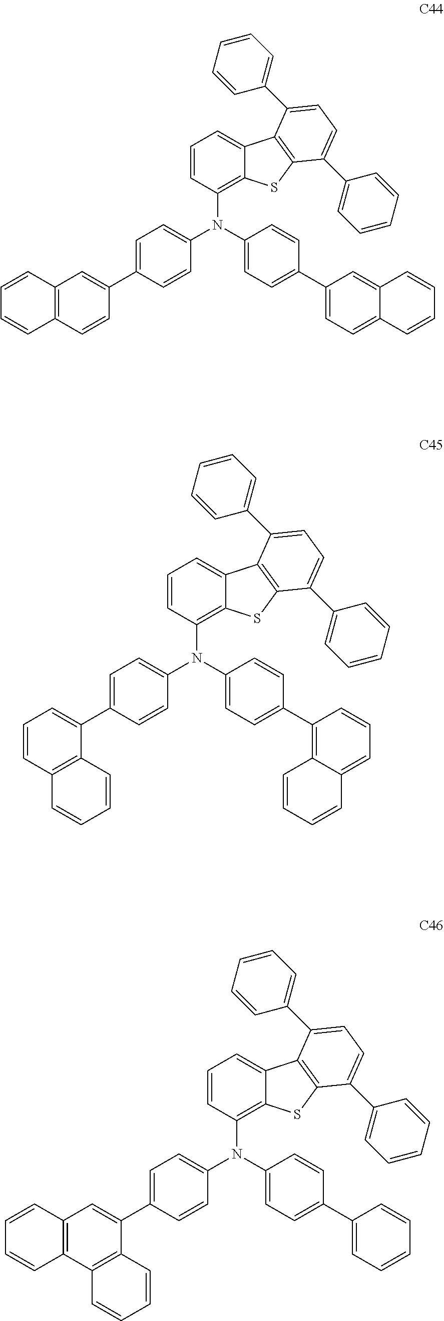 [SMILES: c1ccc(-c2ccc(-c3ccccc3)c3c2sc2c(N(c4ccc(-c5ccc6ccccc6c5)cc4)c4ccc(-c5ccc6ccccc6c5)cc4)cccc23)cc1 ; c1ccc(-c2ccc(-c3ccccc3)c3c2sc2c(N(c4ccc(-c5cccc6ccccc56)cc4)c4ccc(-c5cccc6ccccc56)cc4)cccc23)cc1 ; c1ccc(-c2ccc(N(c3ccc(-c4cc5ccccc5c5ccccc45)cc3)c3cccc4c3sc3c(-c5ccccc5)ccc(-c5ccccc5)c34)cc2)cc1] 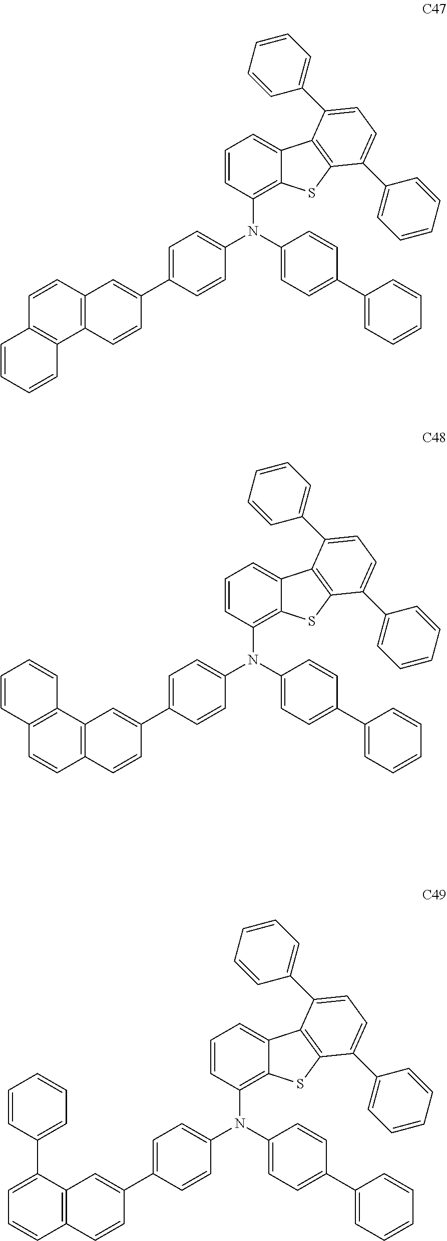 [SMILES: c1ccc(-c2ccc(N(c3ccc(-c4ccc5c(ccc6ccccc65)c4)cc3)c3cccc4c3sc3c(-c5ccccc5)ccc(-c5ccccc5)c34)cc2)cc1 ; c1ccc(-c2ccc(N(c3ccc(-c4ccc5ccc6ccccc6c5c4)cc3)c3cccc4c3sc3c(-c5ccccc5)ccc(-c5ccccc5)c34)cc2)cc1 ; c1ccc(-c2ccc(N(c3ccc(-c4ccc5cccc(-c6ccccc6)c5c4)cc3)c3cccc4c3sc3c(-c5ccccc5)ccc(-c5ccccc5)c34)cc2)cc1] 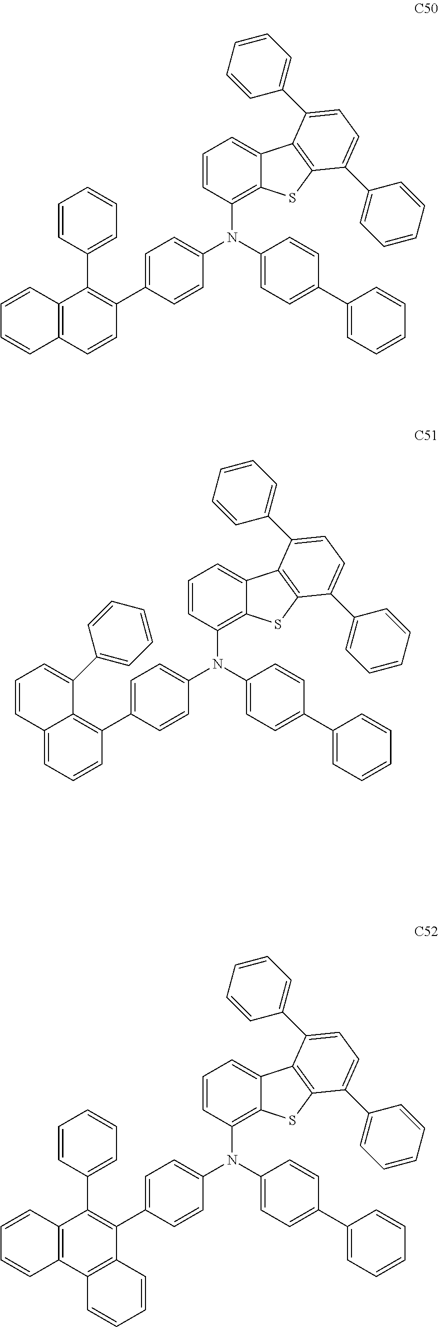 [SMILES: c1ccc(-c2ccc(N(c3ccc(-c4ccc5ccccc5c4-c4ccccc4)cc3)c3cccc4c3sc3c(-c5ccccc5)ccc(-c5ccccc5)c34)cc2)cc1 ; c1ccc(-c2ccc(N(c3ccc(-c4cccc5cccc(-c6ccccc6)c45)cc3)c3cccc4c3sc3c(-c5ccccc5)ccc(-c5ccccc5)c34)cc2)cc1 ; c1ccc(-c2ccc(N(c3ccc(-c4c(-c5ccccc5)c5ccccc5c5ccccc45)cc3)c3cccc4c3sc3c(-c5ccccc5)ccc(-c5ccccc5)c34)cc2)cc1] 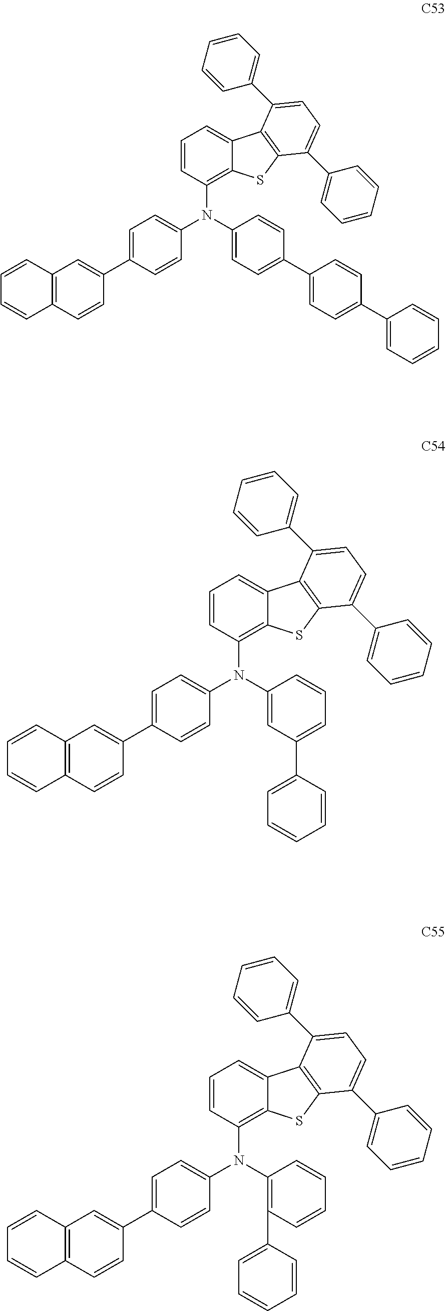 [SMILES: c1ccc(-c2ccc(-c3ccc(N(c4ccc(-c5ccc6ccccc6c5)cc4)c4cccc5c4sc4c(-c6ccccc6)ccc(-c6ccccc6)c45)cc3)cc2)cc1 ; c1ccc(-c2cccc(N(c3ccc(-c4ccc5ccccc5c4)cc3)c3cccc4c3sc3c(-c5ccccc5)ccc(-c5ccccc5)c34)c2)cc1 ; c1ccc(-c2ccccc2N(c2ccc(-c3ccc4ccccc4c3)cc2)c2cccc3c2sc2c(-c4ccccc4)ccc(-c4ccccc4)c23)cc1] 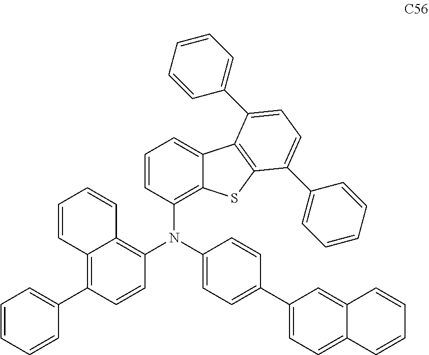 [SMILES: c1ccc(-c2ccc(N(c3ccc(-c4ccc5ccccc5c4)cc3)c3cccc4c3sc3c(-c5ccccc5)ccc(-c5ccccc5)c34)c3ccccc23)cc1] 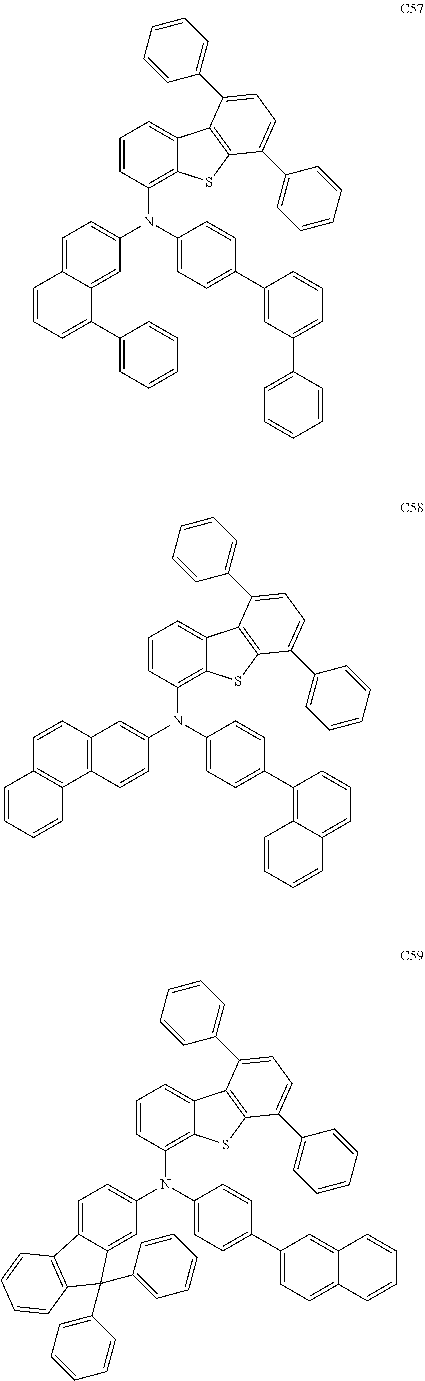 [SMILES: c1ccc(-c2cccc(-c3ccc(N(c4ccc5cccc(-c6ccccc6)c5c4)c4cccc5c4sc4c(-c6ccccc6)ccc(-c6ccccc6)c45)cc3)c2)cc1 ; c1ccc(-c2ccc(-c3ccccc3)c3c2sc2c(N(c4ccc(-c5cccc6ccccc56)cc4)c4ccc5c(ccc6ccccc65)c4)cccc23)cc1 ; c1ccc(-c2ccc(-c3ccccc3)c3c2sc2c(N(c4ccc(-c5ccc6ccccc6c5)cc4)c4ccc5c(c4)C(c4ccccc4)(c4ccccc4)c4ccccc4-5)cccc23)cc1] 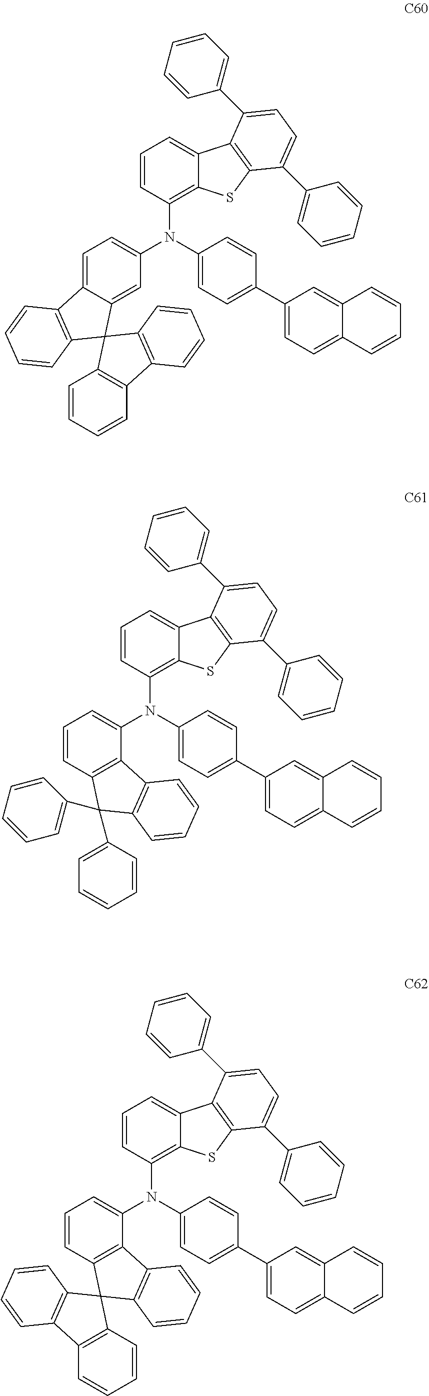 [SMILES: c1ccc(-c2ccc(-c3ccccc3)c3c2sc2c(N(c4ccc(-c5ccc6ccccc6c5)cc4)c4ccc5c(c4)C4(c6ccccc6-c6ccccc64)c4ccccc4-5)cccc23)cc1 ; c1ccc(-c2ccc(-c3ccccc3)c3c2sc2c(N(c4ccc(-c5ccc6ccccc6c5)cc4)c4cccc5c4-c4ccccc4C5(c4ccccc4)c4ccccc4)cccc23)cc1 ; c1ccc(-c2ccc(-c3ccccc3)c3c2sc2c(N(c4ccc(-c5ccc6ccccc6c5)cc4)c4cccc5c4-c4ccccc4C54c5ccccc5-c5ccccc54)cccc23)cc1] 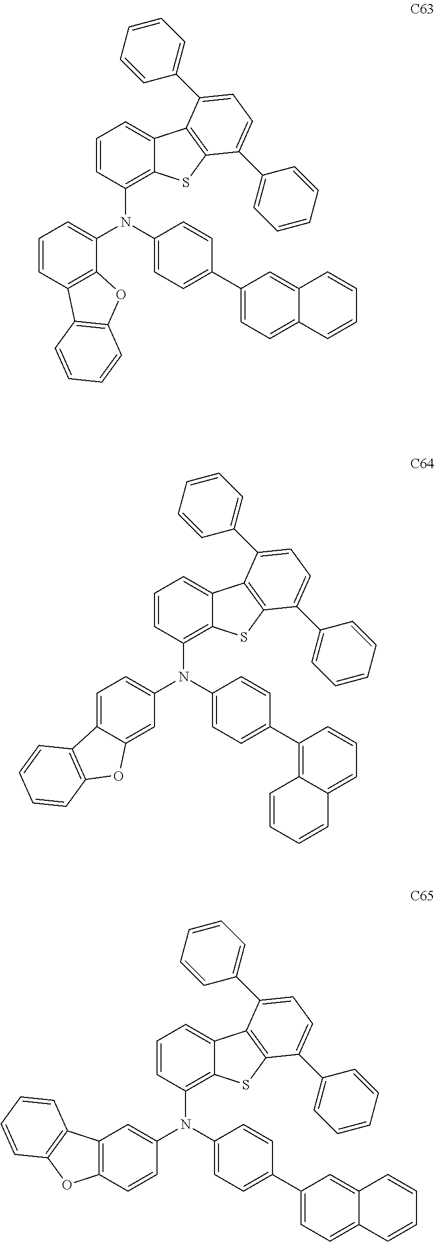 [SMILES: c1ccc(-c2ccc(-c3ccccc3)c3c2sc2c(N(c4ccc(-c5ccc6ccccc6c5)cc4)c4cccc5c4oc4ccccc45)cccc23)cc1 ; c1ccc(-c2ccc(-c3ccccc3)c3c2sc2c(N(c4ccc(-c5cccc6ccccc56)cc4)c4ccc5c(c4)oc4ccccc45)cccc23)cc1 ; c1ccc(-c2ccc(-c3ccccc3)c3c2sc2c(N(c4ccc(-c5ccc6ccccc6c5)cc4)c4ccc5oc6ccccc6c5c4)cccc23)cc1] 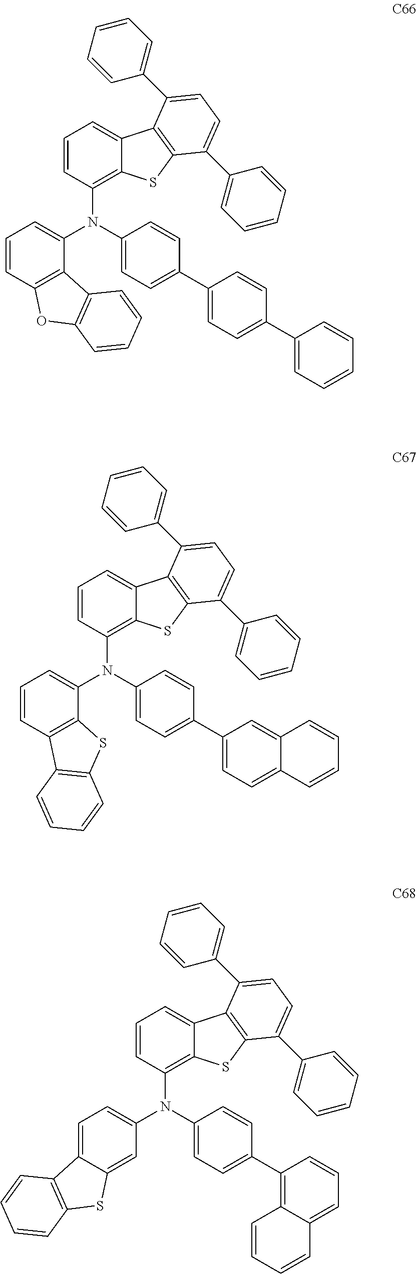 [SMILES: c1ccc(-c2ccc(-c3ccc(N(c4cccc5c4sc4c(-c6ccccc6)ccc(-c6ccccc6)c45)c4cccc5oc6ccccc6c45)cc3)cc2)cc1 ; c1ccc(-c2ccc(-c3ccccc3)c3c2sc2c(N(c4ccc(-c5ccc6ccccc6c5)cc4)c4cccc5c4sc4ccccc45)cccc23)cc1 ; c1ccc(-c2ccc(-c3ccccc3)c3c2sc2c(N(c4ccc(-c5cccc6ccccc56)cc4)c4ccc5c(c4)sc4ccccc45)cccc23)cc1] 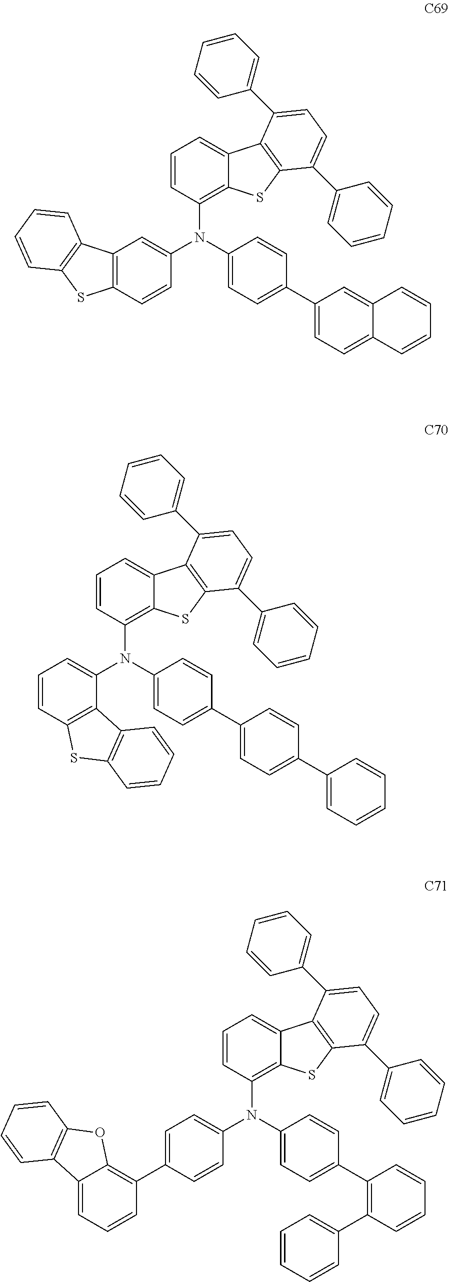 [SMILES: c1ccc(-c2ccc(-c3ccccc3)c3c2sc2c(N(c4ccc(-c5ccc6ccccc6c5)cc4)c4ccc5sc6ccccc6c5c4)cccc23)cc1 ; c1ccc(-c2ccc(-c3ccc(N(c4cccc5c4sc4c(-c6ccccc6)ccc(-c6ccccc6)c45)c4cccc5sc6ccccc6c45)cc3)cc2)cc1 ; c1ccc(-c2ccccc2-c2ccc(N(c3ccc(-c4cccc5c4oc4ccccc45)cc3)c3cccc4c3sc3c(-c5ccccc5)ccc(-c5ccccc5)c34)cc2)cc1] 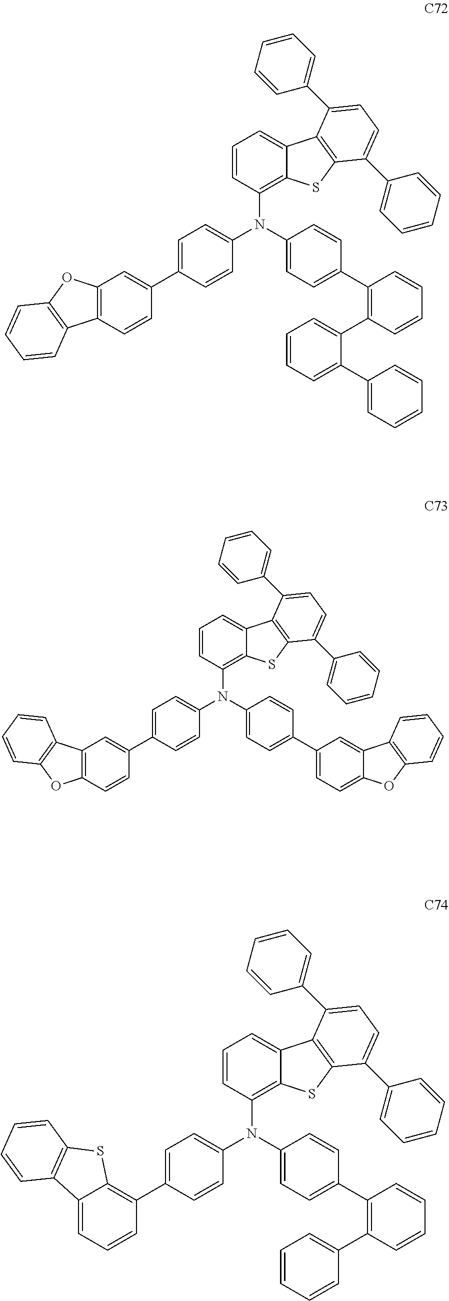 [SMILES: c1ccc(-c2ccccc2-c2ccccc2-c2ccc(N(c3ccc(-c4ccc5c(c4)oc4ccccc45)cc3)c3cccc4c3sc3c(-c5ccccc5)ccc(-c5ccccc5)c34)cc2)cc1 ; c1ccc(-c2ccc(-c3ccccc3)c3c2sc2c(N(c4ccc(-c5ccc6oc7ccccc7c6c5)cc4)c4ccc(-c5ccc6oc7ccccc7c6c5)cc4)cccc23)cc1 ; c1ccc(-c2ccccc2-c2ccc(N(c3ccc(-c4cccc5c4sc4ccccc45)cc3)c3cccc4c3sc3c(-c5ccccc5)ccc(-c5ccccc5)c34)cc2)cc1] 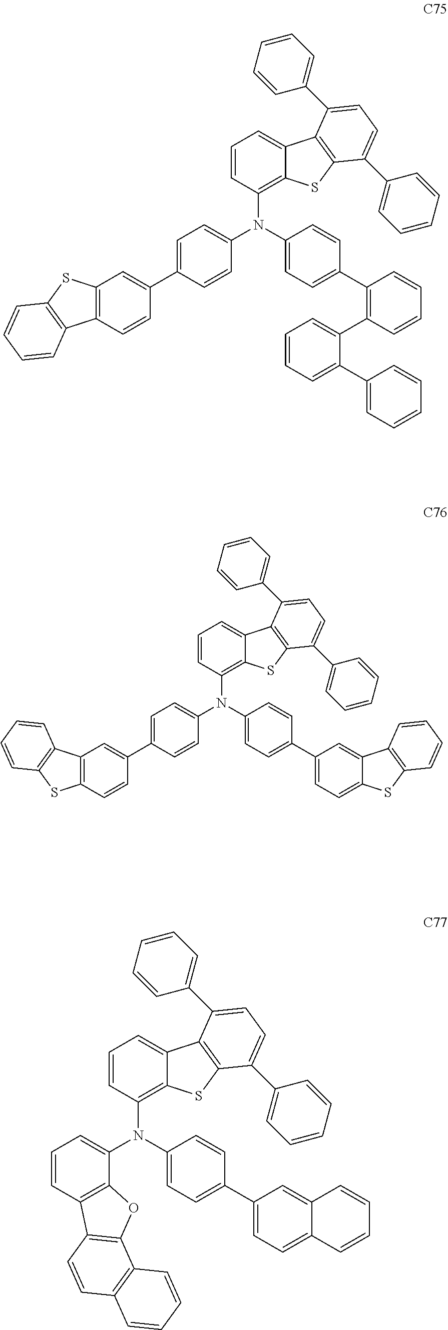 [SMILES: c1ccc(-c2ccccc2-c2ccccc2-c2ccc(N(c3ccc(-c4ccc5c(c4)sc4ccccc45)cc3)c3cccc4c3sc3c(-c5ccccc5)ccc(-c5ccccc5)c34)cc2)cc1 ; c1ccc(-c2ccc(-c3ccccc3)c3c2sc2c(N(c4ccc(-c5ccc6sc7ccccc7c6c5)cc4)c4ccc(-c5ccc6sc7ccccc7c6c5)cc4)cccc23)cc1 ; c1ccc(-c2ccc(-c3ccccc3)c3c2sc2c(N(c4ccc(-c5ccc6ccccc6c5)cc4)c4cccc5c4oc4c6ccccc6ccc54)cccc23)cc1] 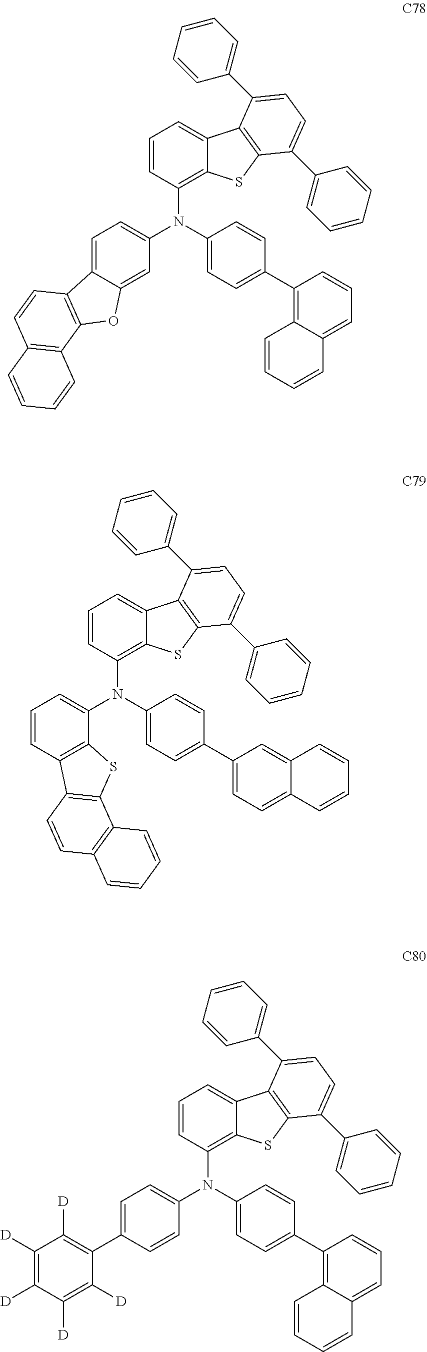 [SMILES: c1ccc(-c2ccc(-c3ccccc3)c3c2sc2c(N(c4ccc(-c5cccc6ccccc56)cc4)c4ccc5c(c4)oc4c6ccccc6ccc54)cccc23)cc1 ; c1ccc(-c2ccc(-c3ccccc3)c3c2sc2c(N(c4ccc(-c5ccc6ccccc6c5)cc4)c4cccc5c4sc4c6ccccc6ccc54)cccc23)cc1 ; [2H]c1c([2H])c([2H])c(-c2ccc(N(c3ccc(-c4cccc5ccccc45)cc3)c3cccc4c3sc3c(-c5ccccc5)ccc(-c5ccccc5)c34)cc2)c([2H])c1[2H]] 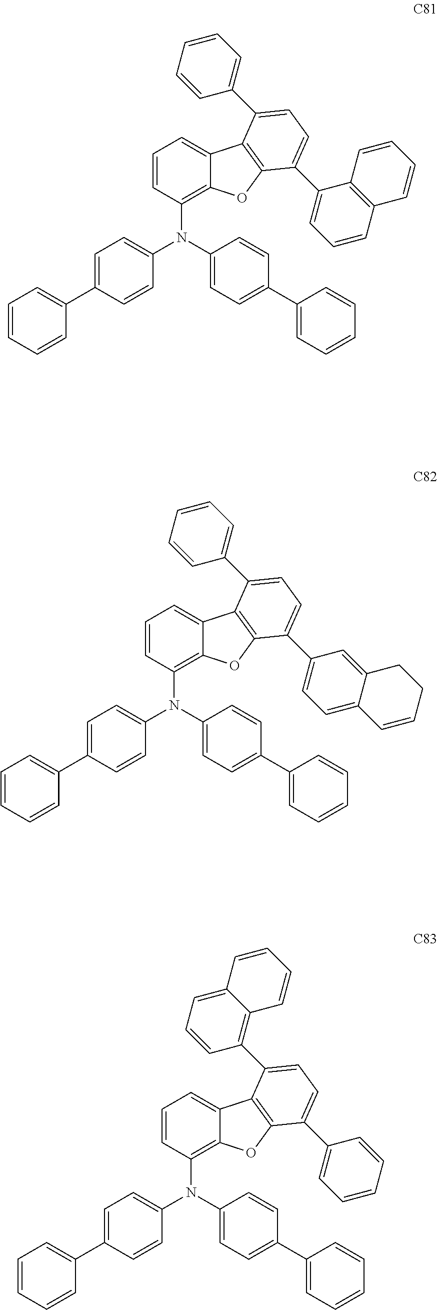 [SMILES: c1ccc(-c2ccc(N(c3ccc(-c4ccccc4)cc3)c3cccc4c3oc3c(-c5cccc6ccccc56)ccc(-c5ccccc5)c34)cc2)cc1 ; C1=Cc2ccc(-c3ccc(-c4ccccc4)c4c3oc3c(N(c5ccc(-c6ccccc6)cc5)c5ccc(-c6ccccc6)cc5)cccc34)cc2CC1 ; c1ccc(-c2ccc(N(c3ccc(-c4ccccc4)cc3)c3cccc4c3oc3c(-c5ccccc5)ccc(-c5cccc6ccccc56)c34)cc2)cc1] 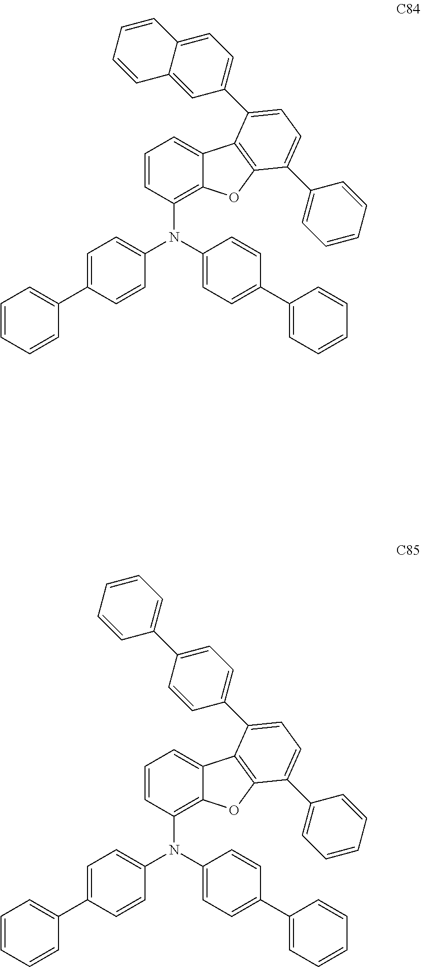 [SMILES: c1ccc(-c2ccc(N(c3ccc(-c4ccccc4)cc3)c3cccc4c3oc3c(-c5ccccc5)ccc(-c5ccc6ccccc6c5)c34)cc2)cc1 ; c1ccc(-c2ccc(-c3ccc(-c4ccccc4)c4oc5c(N(c6ccc(-c7ccccc7)cc6)c6ccc(-c7ccccc7)cc6)cccc5c34)cc2)cc1] 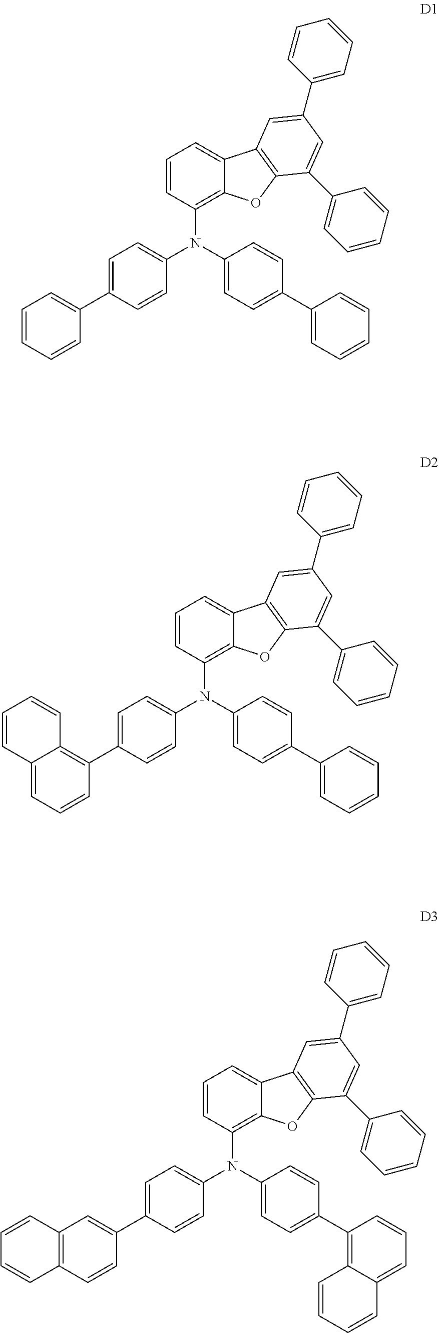 [SMILES: c1ccc(-c2ccc(N(c3ccc(-c4ccccc4)cc3)c3cccc4c3oc3c(-c5ccccc5)cc(-c5ccccc5)cc34)cc2)cc1 ; c1ccc(-c2ccc(N(c3ccc(-c4cccc5ccccc45)cc3)c3cccc4c3oc3c(-c5ccccc5)cc(-c5ccccc5)cc34)cc2)cc1 ; c1ccc(-c2cc(-c3ccccc3)c3oc4c(N(c5ccc(-c6ccc7ccccc7c6)cc5)c5ccc(-c6cccc7ccccc67)cc5)cccc4c3c2)cc1] 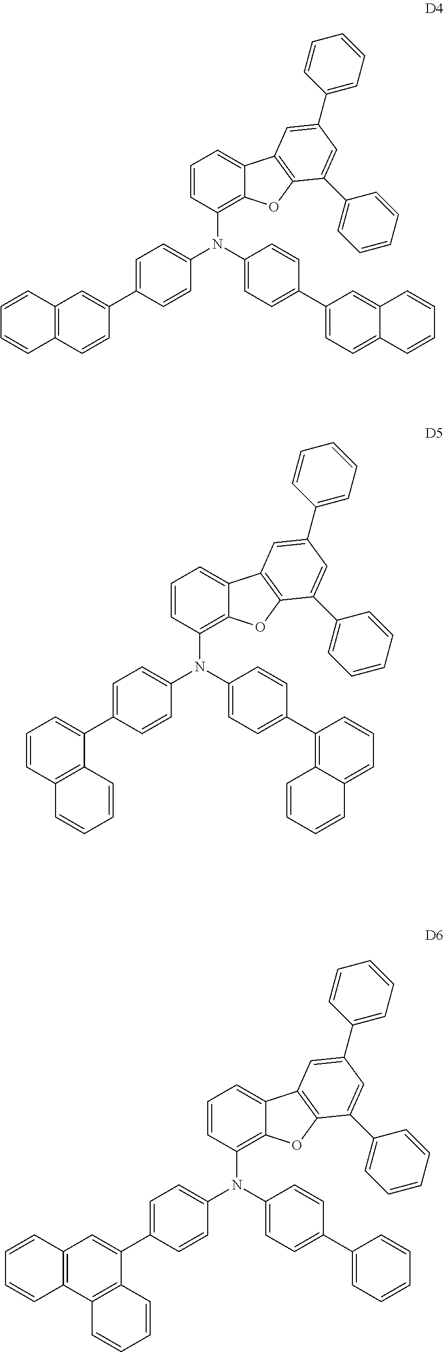 [SMILES: c1ccc(-c2cc(-c3ccccc3)c3oc4c(N(c5ccc(-c6ccc7ccccc7c6)cc5)c5ccc(-c6ccc7ccccc7c6)cc5)cccc4c3c2)cc1 ; c1ccc(-c2cc(-c3ccccc3)c3oc4c(N(c5ccc(-c6cccc7ccccc67)cc5)c5ccc(-c6cccc7ccccc67)cc5)cccc4c3c2)cc1 ; c1ccc(-c2ccc(N(c3ccc(-c4cc5ccccc5c5ccccc45)cc3)c3cccc4c3oc3c(-c5ccccc5)cc(-c5ccccc5)cc34)cc2)cc1] 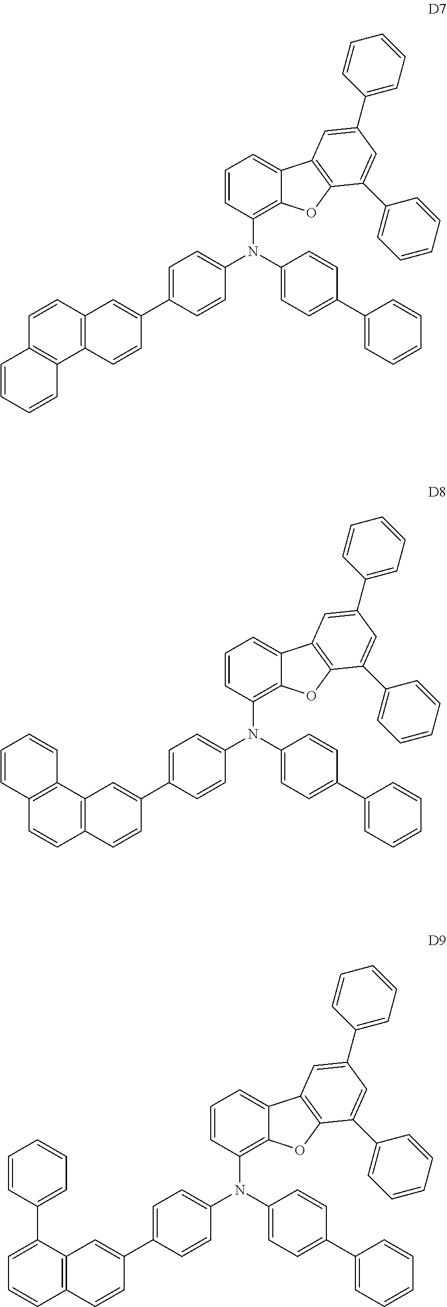 [SMILES: c1ccc(-c2ccc(N(c3ccc(-c4ccc5c(ccc6ccccc65)c4)cc3)c3cccc4c3oc3c(-c5ccccc5)cc(-c5ccccc5)cc34)cc2)cc1 ; c1ccc(-c2ccc(N(c3ccc(-c4ccc5ccc6ccccc6c5c4)cc3)c3cccc4c3oc3c(-c5ccccc5)cc(-c5ccccc5)cc34)cc2)cc1 ; c1ccc(-c2ccc(N(c3ccc(-c4ccc5cccc(-c6ccccc6)c5c4)cc3)c3cccc4c3oc3c(-c5ccccc5)cc(-c5ccccc5)cc34)cc2)cc1] 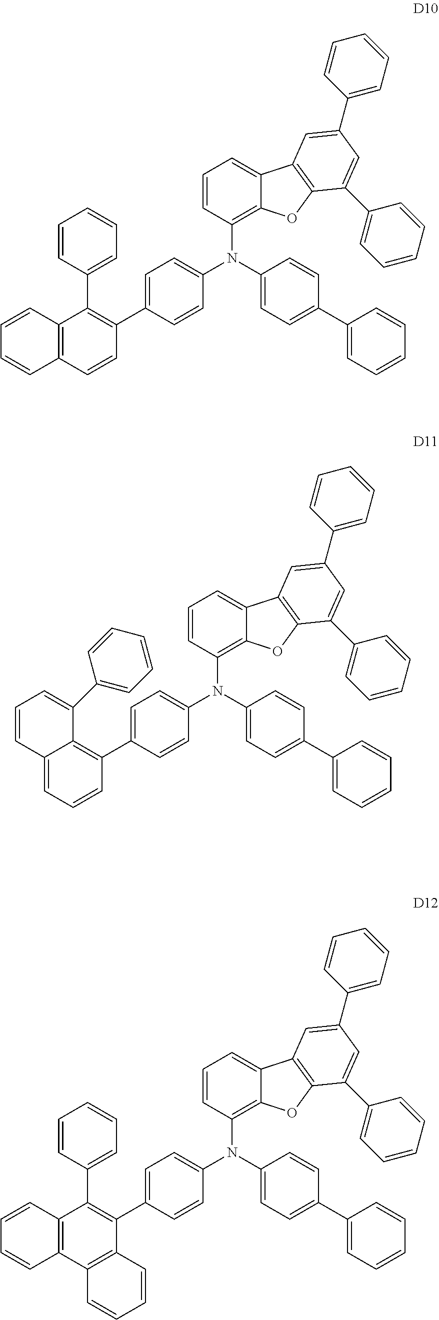 [SMILES: c1ccc(-c2ccc(N(c3ccc(-c4ccc5ccccc5c4-c4ccccc4)cc3)c3cccc4c3oc3c(-c5ccccc5)cc(-c5ccccc5)cc34)cc2)cc1 ; c1ccc(-c2ccc(N(c3ccc(-c4cccc5cccc(-c6ccccc6)c45)cc3)c3cccc4c3oc3c(-c5ccccc5)cc(-c5ccccc5)cc34)cc2)cc1 ; c1ccc(-c2ccc(N(c3ccc(-c4c(-c5ccccc5)c5ccccc5c5ccccc45)cc3)c3cccc4c3oc3c(-c5ccccc5)cc(-c5ccccc5)cc34)cc2)cc1] 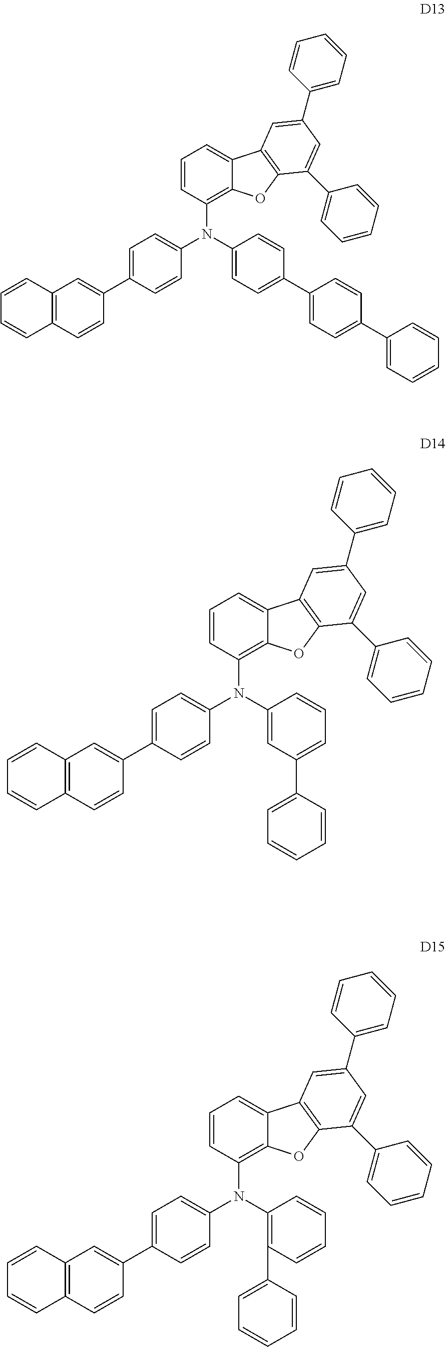 [SMILES: c1ccc(-c2ccc(-c3ccc(N(c4ccc(-c5ccc6ccccc6c5)cc4)c4cccc5c4oc4c(-c6ccccc6)cc(-c6ccccc6)cc45)cc3)cc2)cc1 ; c1ccc(-c2cccc(N(c3ccc(-c4ccc5ccccc5c4)cc3)c3cccc4c3oc3c(-c5ccccc5)cc(-c5ccccc5)cc34)c2)cc1 ; c1ccc(-c2cc(-c3ccccc3)c3oc4c(N(c5ccc(-c6ccc7ccccc7c6)cc5)c5ccccc5-c5ccccc5)cccc4c3c2)cc1] 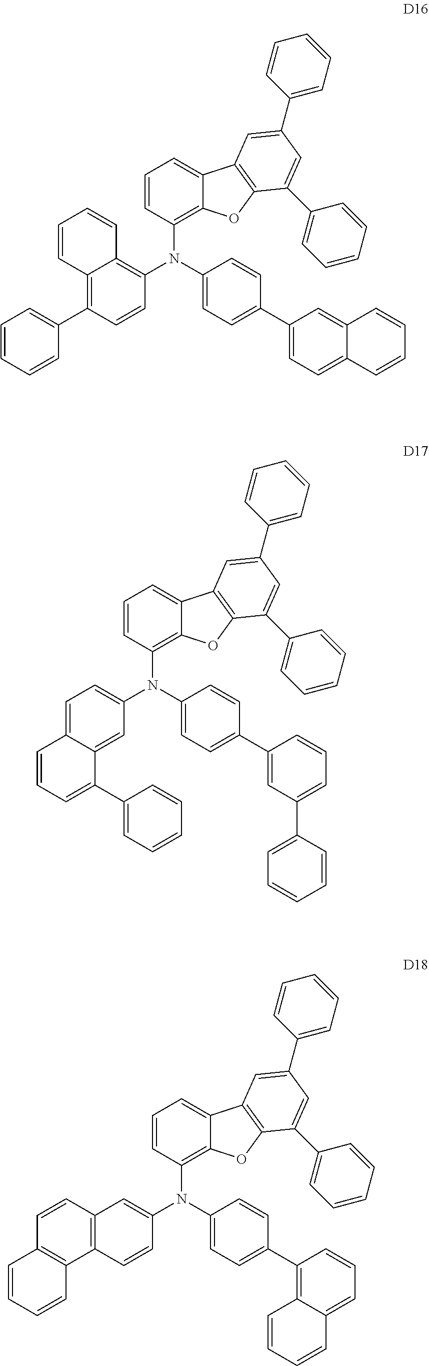 [SMILES: c1ccc(-c2cc(-c3ccccc3)c3oc4c(N(c5ccc(-c6ccc7ccccc7c6)cc5)c5ccc(-c6ccccc6)c6ccccc56)cccc4c3c2)cc1 ; c1ccc(-c2cccc(-c3ccc(N(c4ccc5cccc(-c6ccccc6)c5c4)c4cccc5c4oc4c(-c6ccccc6)cc(-c6ccccc6)cc45)cc3)c2)cc1 ; c1ccc(-c2cc(-c3ccccc3)c3oc4c(N(c5ccc(-c6cccc7ccccc67)cc5)c5ccc6c(ccc7ccccc76)c5)cccc4c3c2)cc1] 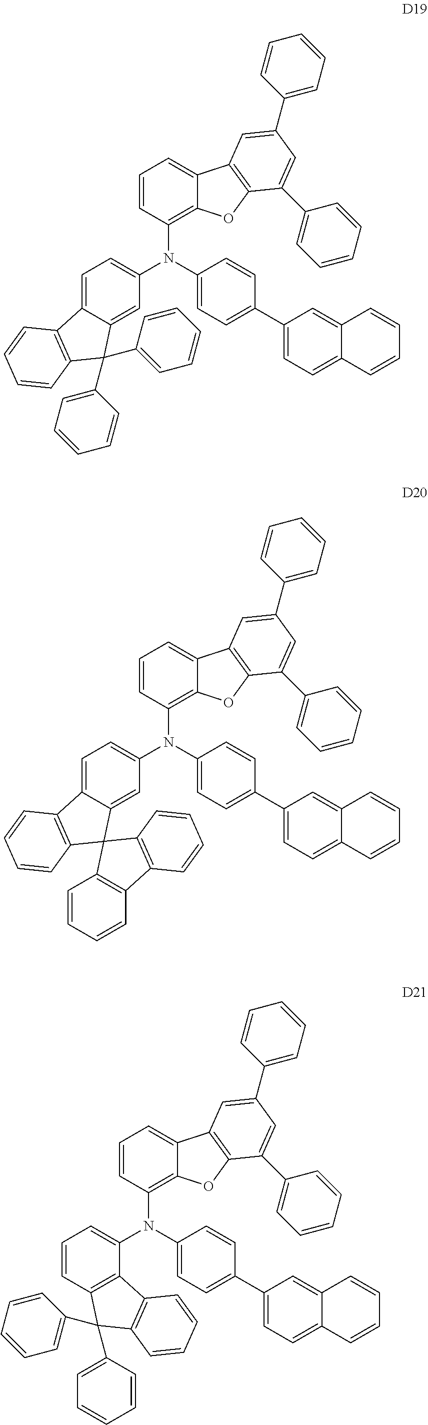 [SMILES: c1ccc(-c2cc(-c3ccccc3)c3oc4c(N(c5ccc(-c6ccc7ccccc7c6)cc5)c5ccc6c(c5)C(c5ccccc5)(c5ccccc5)c5ccccc5-6)cccc4c3c2)cc1 ; c1ccc(-c2cc(-c3ccccc3)c3oc4c(N(c5ccc(-c6ccc7ccccc7c6)cc5)c5ccc6c(c5)C5(c7ccccc7-c7ccccc75)c5ccccc5-6)cccc4c3c2)cc1 ; c1ccc(-c2cc(-c3ccccc3)c3oc4c(N(c5ccc(-c6ccc7ccccc7c6)cc5)c5cccc6c5-c5ccccc5C6(c5ccccc5)c5ccccc5)cccc4c3c2)cc1] 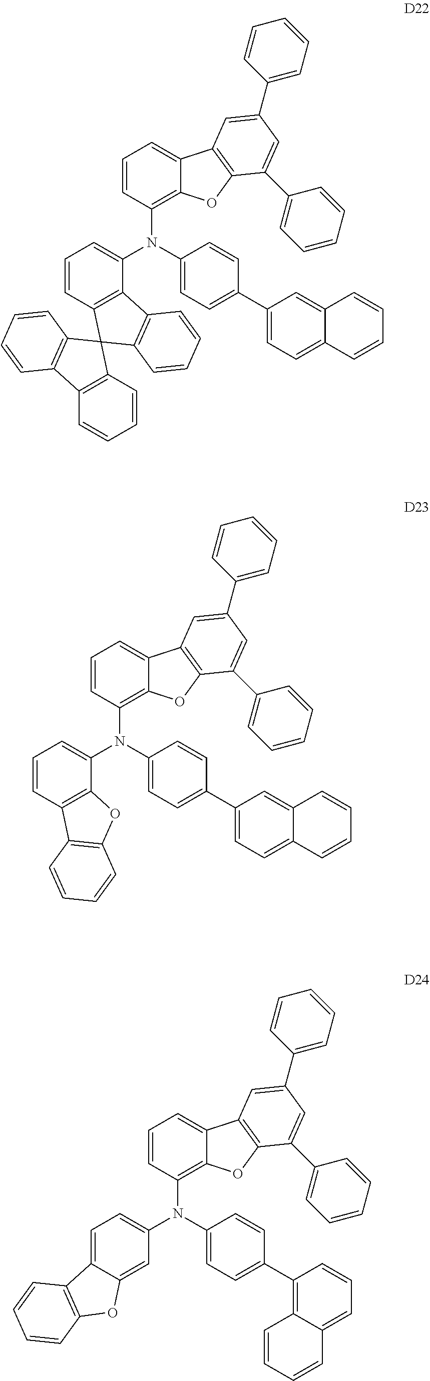 [SMILES: c1ccc(-c2cc(-c3ccccc3)c3oc4c(N(c5ccc(-c6ccc7ccccc7c6)cc5)c5cccc6c5-c5ccccc5C65c6ccccc6-c6ccccc65)cccc4c3c2)cc1 ; c1ccc(-c2cc(-c3ccccc3)c3oc4c(N(c5ccc(-c6ccc7ccccc7c6)cc5)c5cccc6c5oc5ccccc56)cccc4c3c2)cc1 ; c1ccc(-c2cc(-c3ccccc3)c3oc4c(N(c5ccc(-c6cccc7ccccc67)cc5)c5ccc6c(c5)oc5ccccc56)cccc4c3c2)cc1] 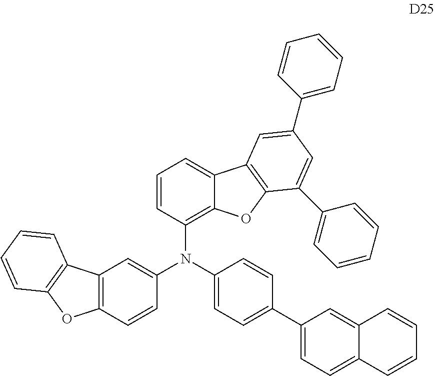 [SMILES: c1ccc(-c2cc(-c3ccccc3)c3oc4c(N(c5ccc(-c6ccc7ccccc7c6)cc5)c5ccc6oc7ccccc7c6c5)cccc4c3c2)cc1] 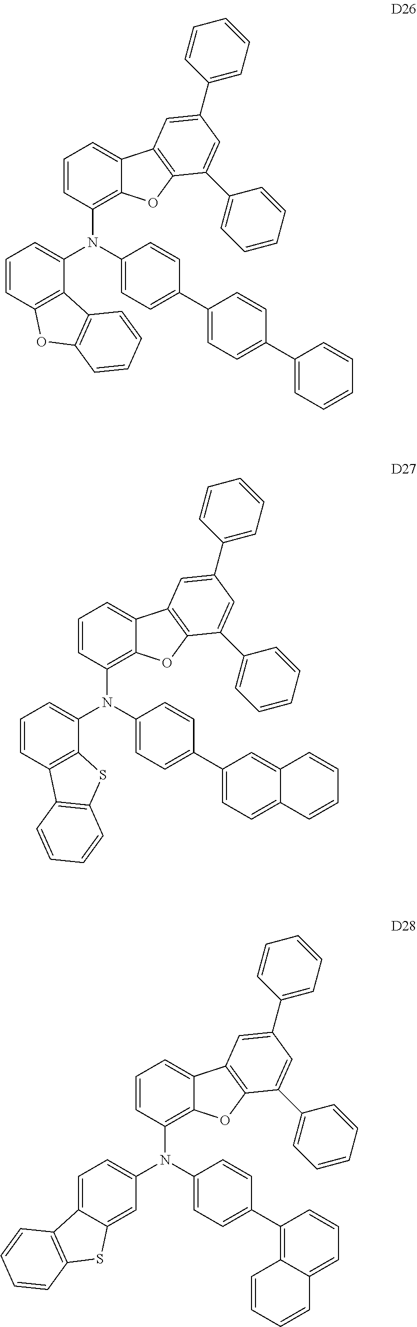 [SMILES: c1ccc(-c2ccc(-c3ccc(N(c4cccc5c4oc4c(-c6ccccc6)cc(-c6ccccc6)cc45)c4cccc5oc6ccccc6c45)cc3)cc2)cc1 ; c1ccc(-c2cc(-c3ccccc3)c3oc4c(N(c5ccc(-c6ccc7ccccc7c6)cc5)c5cccc6c5sc5ccccc56)cccc4c3c2)cc1 ; c1ccc(-c2cc(-c3ccccc3)c3oc4c(N(c5ccc(-c6cccc7ccccc67)cc5)c5ccc6c(c5)sc5ccccc56)cccc4c3c2)cc1] 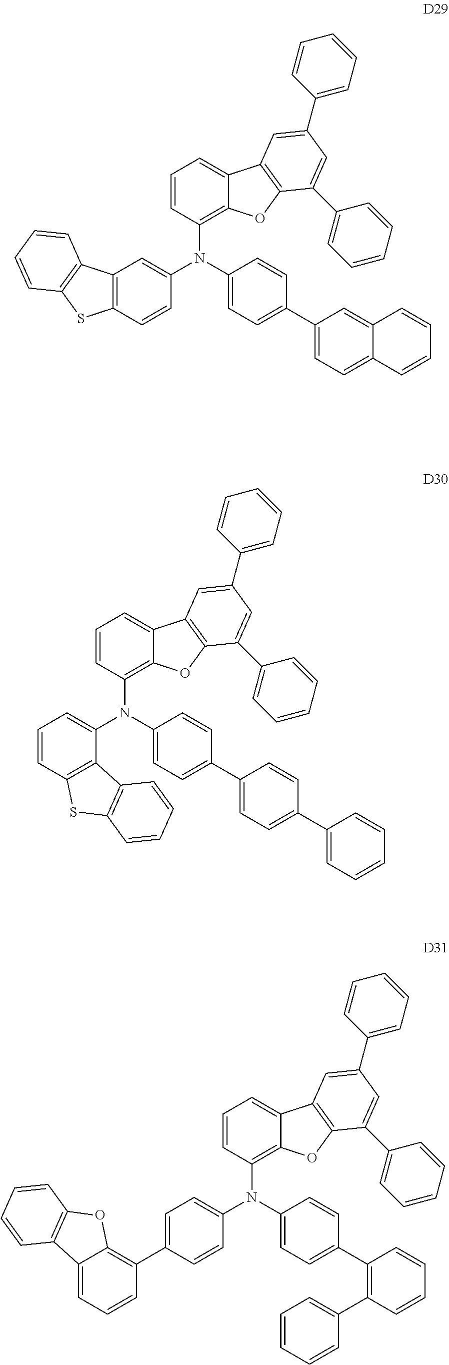 [SMILES: c1ccc(-c2cc(-c3ccccc3)c3oc4c(N(c5ccc(-c6ccc7ccccc7c6)cc5)c5ccc6sc7ccccc7c6c5)cccc4c3c2)cc1 ; c1ccc(-c2ccc(-c3ccc(N(c4cccc5c4oc4c(-c6ccccc6)cc(-c6ccccc6)cc45)c4cccc5sc6ccccc6c45)cc3)cc2)cc1 ; c1ccc(-c2cc(-c3ccccc3)c3oc4c(N(c5ccc(-c6ccccc6-c6ccccc6)cc5)c5ccc(-c6cccc7c6oc6ccccc67)cc5)cccc4c3c2)cc1] 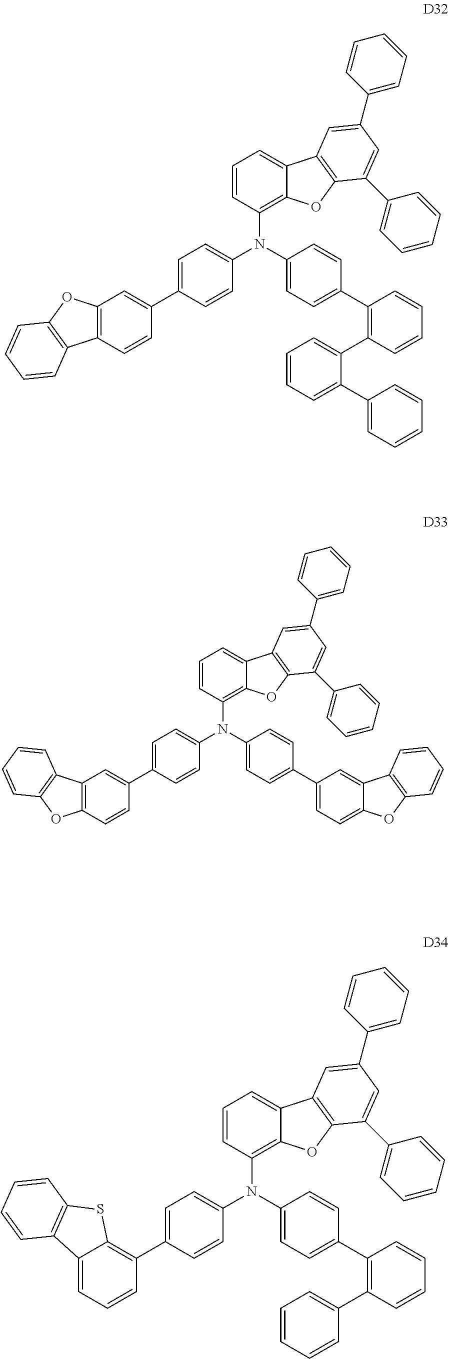 [SMILES: c1ccc(-c2cc(-c3ccccc3)c3oc4c(N(c5ccc(-c6ccc7c(c6)oc6ccccc67)cc5)c5ccc(-c6ccccc6-c6ccccc6-c6ccccc6)cc5)cccc4c3c2)cc1 ; c1ccc(-c2cc(-c3ccccc3)c3oc4c(N(c5ccc(-c6ccc7oc8ccccc8c7c6)cc5)c5ccc(-c6ccc7oc8ccccc8c7c6)cc5)cccc4c3c2)cc1 ; c1ccc(-c2cc(-c3ccccc3)c3oc4c(N(c5ccc(-c6ccccc6-c6ccccc6)cc5)c5ccc(-c6cccc7c6sc6ccccc67)cc5)cccc4c3c2)cc1] 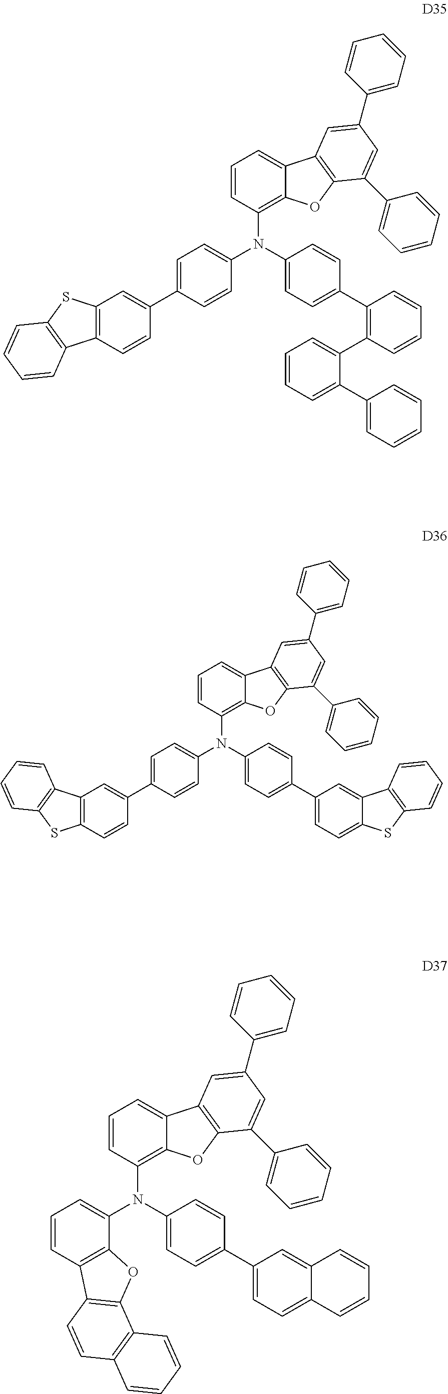 [SMILES: c1ccc(-c2cc(-c3ccccc3)c3oc4c(N(c5ccc(-c6ccc7c(c6)sc6ccccc67)cc5)c5ccc(-c6ccccc6-c6ccccc6-c6ccccc6)cc5)cccc4c3c2)cc1 ; c1ccc(-c2cc(-c3ccccc3)c3oc4c(N(c5ccc(-c6ccc7sc8ccccc8c7c6)cc5)c5ccc(-c6ccc7sc8ccccc8c7c6)cc5)cccc4c3c2)cc1 ; c1ccc(-c2cc(-c3ccccc3)c3oc4c(N(c5ccc(-c6ccc7ccccc7c6)cc5)c5cccc6c5oc5c7ccccc7ccc65)cccc4c3c2)cc1] 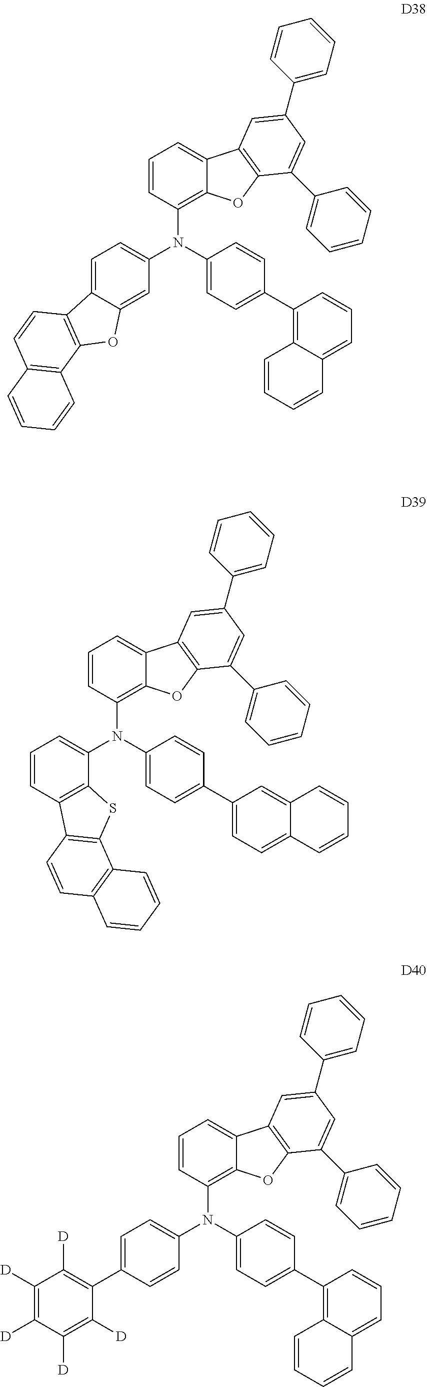 [SMILES: c1ccc(-c2cc(-c3ccccc3)c3oc4c(N(c5ccc(-c6cccc7ccccc67)cc5)c5ccc6c(c5)oc5c7ccccc7ccc65)cccc4c3c2)cc1 ; c1ccc(-c2cc(-c3ccccc3)c3oc4c(N(c5ccc(-c6ccc7ccccc7c6)cc5)c5cccc6c5sc5c7ccccc7ccc65)cccc4c3c2)cc1 ; [2H]c1c([2H])c([2H])c(-c2ccc(N(c3ccc(-c4cccc5ccccc45)cc3)c3cccc4c3oc3c(-c5ccccc5)cc(-c5ccccc5)cc34)cc2)c([2H])c1[2H]] 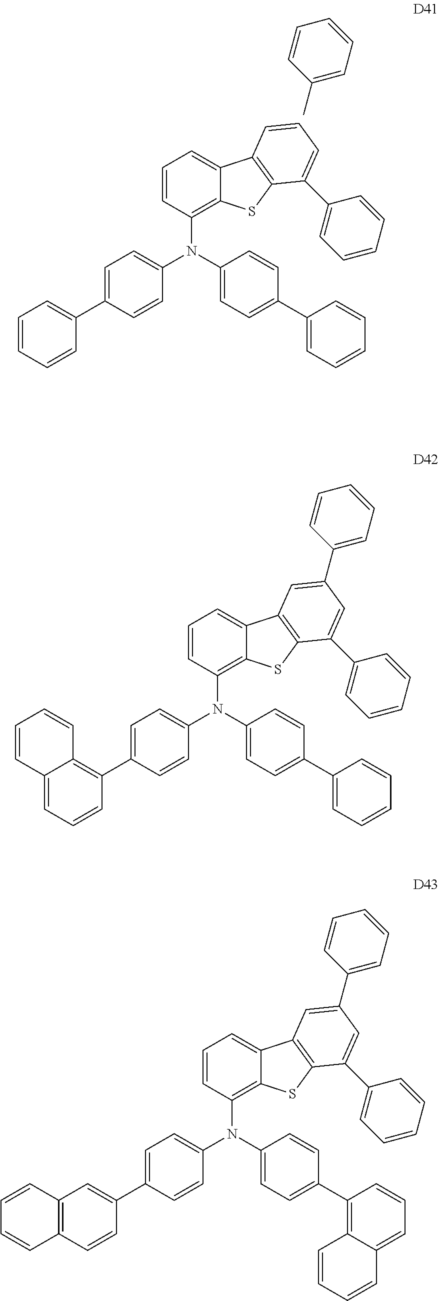 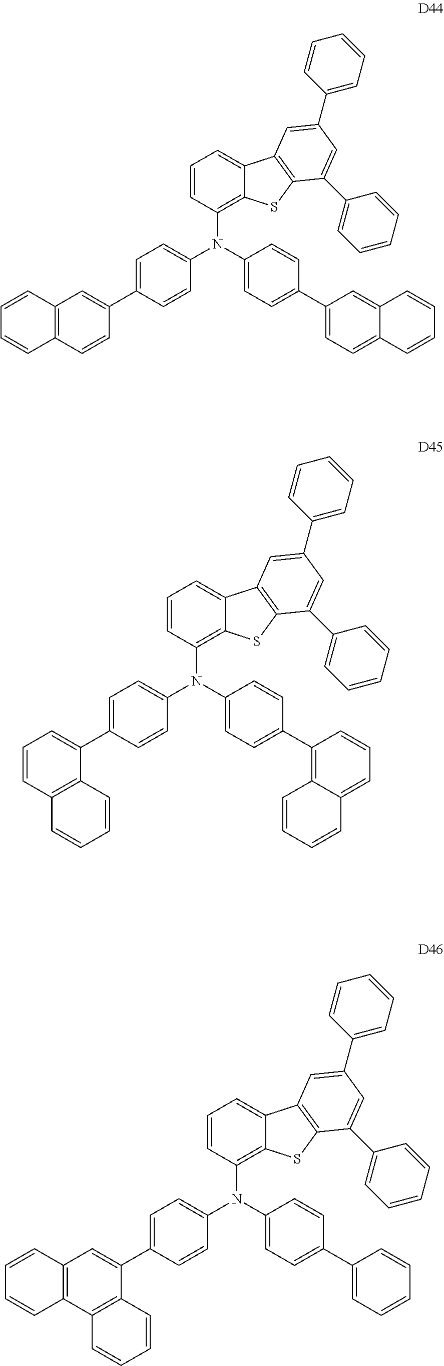 [SMILES: c1ccc(-c2cc(-c3ccccc3)c3sc4c(N(c5ccc(-c6ccc7ccccc7c6)cc5)c5ccc(-c6ccc7ccccc7c6)cc5)cccc4c3c2)cc1 ; c1ccc(-c2cc(-c3ccccc3)c3sc4c(N(c5ccc(-c6cccc7ccccc67)cc5)c5ccc(-c6cccc7ccccc67)cc5)cccc4c3c2)cc1 ; c1ccc(-c2ccc(N(c3ccc(-c4cc5ccccc5c5ccccc45)cc3)c3cccc4c3sc3c(-c5ccccc5)cc(-c5ccccc5)cc34)cc2)cc1] 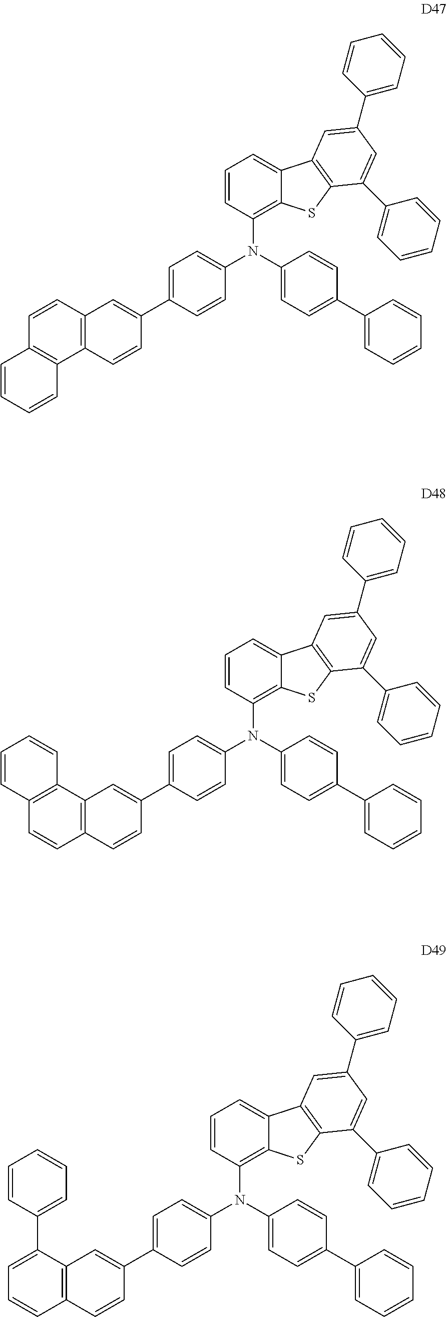 [SMILES: c1ccc(-c2ccc(N(c3ccc(-c4ccc5c(ccc6ccccc65)c4)cc3)c3cccc4c3sc3c(-c5ccccc5)cc(-c5ccccc5)cc34)cc2)cc1 ; c1ccc(-c2ccc(N(c3ccc(-c4ccc5ccc6ccccc6c5c4)cc3)c3cccc4c3sc3c(-c5ccccc5)cc(-c5ccccc5)cc34)cc2)cc1 ; c1ccc(-c2ccc(N(c3ccc(-c4ccc5cccc(-c6ccccc6)c5c4)cc3)c3cccc4c3sc3c(-c5ccccc5)cc(-c5ccccc5)cc34)cc2)cc1] 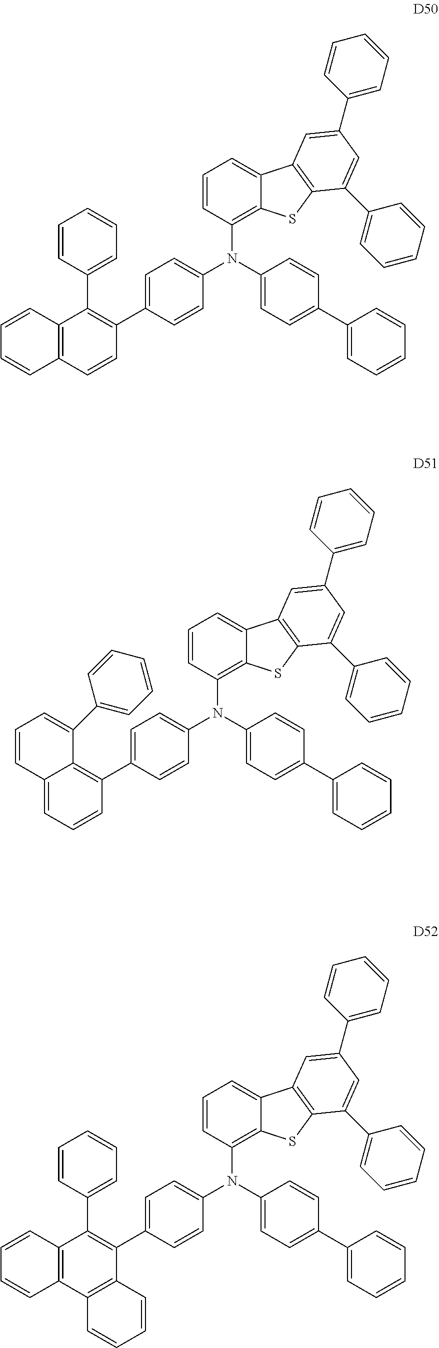 [SMILES: c1ccc(-c2ccc(N(c3ccc(-c4ccc5ccccc5c4-c4ccccc4)cc3)c3cccc4c3sc3c(-c5ccccc5)cc(-c5ccccc5)cc34)cc2)cc1 ; c1ccc(-c2ccc(N(c3ccc(-c4cccc5cccc(-c6ccccc6)c45)cc3)c3cccc4c3sc3c(-c5ccccc5)cc(-c5ccccc5)cc34)cc2)cc1 ; c1ccc(-c2ccc(N(c3ccc(-c4c(-c5ccccc5)c5ccccc5c5ccccc45)cc3)c3cccc4c3sc3c(-c5ccccc5)cc(-c5ccccc5)cc34)cc2)cc1] 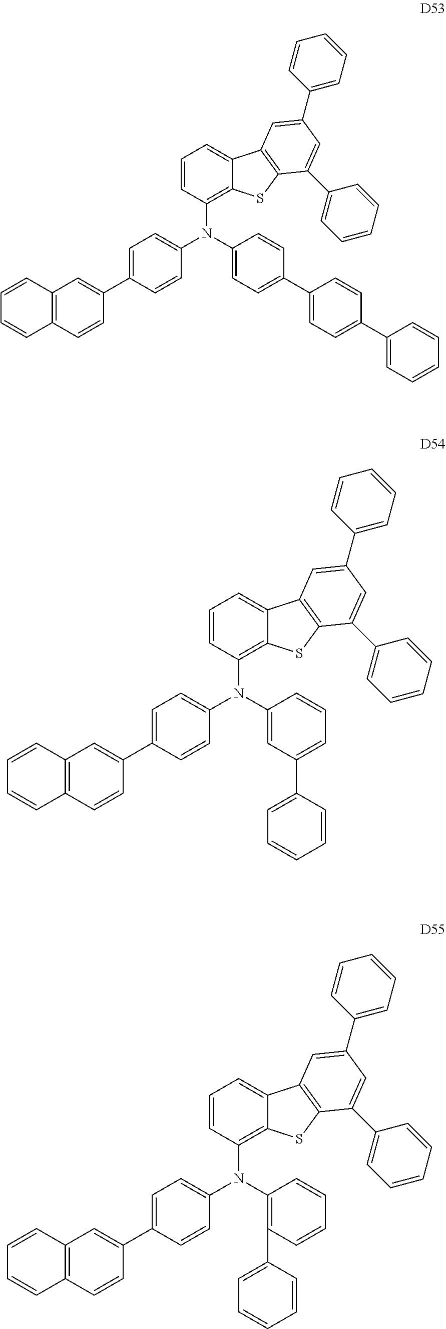 [SMILES: c1ccc(-c2ccc(-c3ccc(N(c4ccc(-c5ccc6ccccc6c5)cc4)c4cccc5c4sc4c(-c6ccccc6)cc(-c6ccccc6)cc45)cc3)cc2)cc1 ; c1ccc(-c2cccc(N(c3ccc(-c4ccc5ccccc5c4)cc3)c3cccc4c3sc3c(-c5ccccc5)cc(-c5ccccc5)cc34)c2)cc1 ; c1ccc(-c2cc(-c3ccccc3)c3sc4c(N(c5ccc(-c6ccc7ccccc7c6)cc5)c5ccccc5-c5ccccc5)cccc4c3c2)cc1] 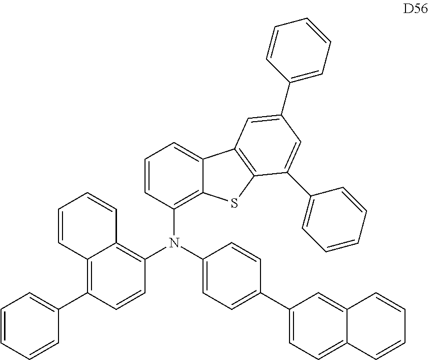 [SMILES: c1ccc(-c2cc(-c3ccccc3)c3sc4c(N(c5ccc(-c6ccc7ccccc7c6)cc5)c5ccc(-c6ccccc6)c6ccccc56)cccc4c3c2)cc1] 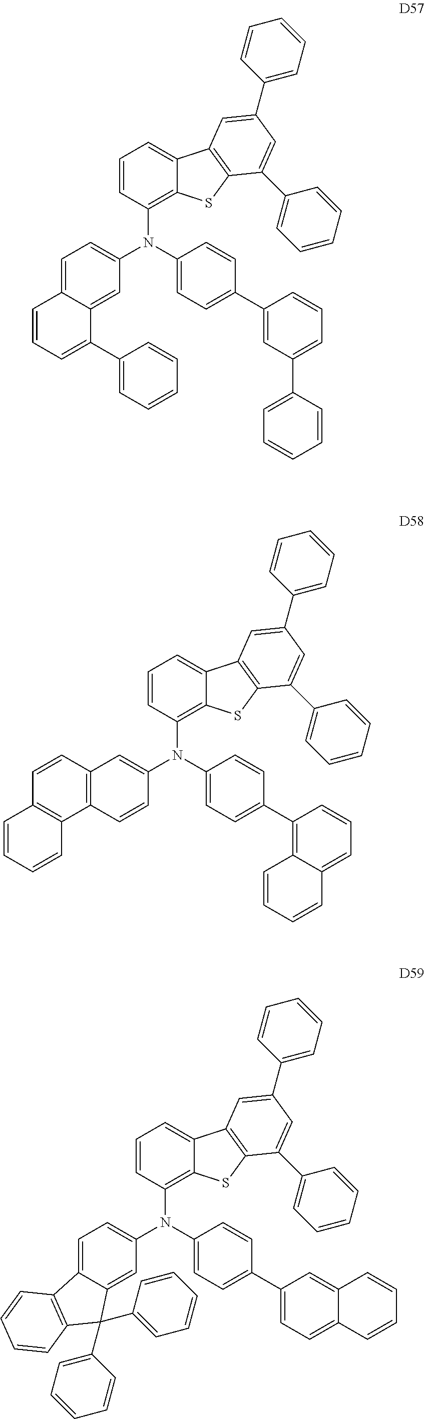 [SMILES: c1ccc(-c2cccc(-c3ccc(N(c4ccc5cccc(-c6ccccc6)c5c4)c4cccc5c4sc4c(-c6ccccc6)cc(-c6ccccc6)cc45)cc3)c2)cc1 ; c1ccc(-c2cc(-c3ccccc3)c3sc4c(N(c5ccc(-c6cccc7ccccc67)cc5)c5ccc6c(ccc7ccccc76)c5)cccc4c3c2)cc1 ; c1ccc(-c2cc(-c3ccccc3)c3sc4c(N(c5ccc(-c6ccc7ccccc7c6)cc5)c5ccc6c(c5)C(c5ccccc5)(c5ccccc5)c5ccccc5-6)cccc4c3c2)cc1] 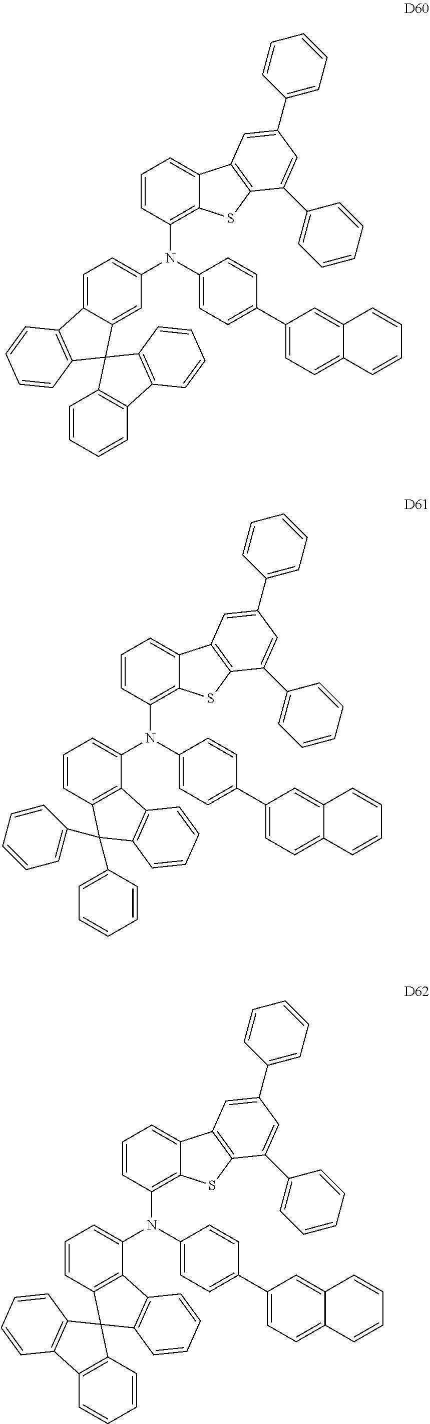 [SMILES: c1ccc(-c2cc(-c3ccccc3)c3sc4c(N(c5ccc(-c6ccc7ccccc7c6)cc5)c5ccc6c(c5)C5(c7ccccc7-c7ccccc75)c5ccccc5-6)cccc4c3c2)cc1 ; c1ccc(-c2cc(-c3ccccc3)c3sc4c(N(c5ccc(-c6ccc7ccccc7c6)cc5)c5cccc6c5-c5ccccc5C6(c5ccccc5)c5ccccc5)cccc4c3c2)cc1 ; c1ccc(-c2cc(-c3ccccc3)c3sc4c(N(c5ccc(-c6ccc7ccccc7c6)cc5)c5cccc6c5-c5ccccc5C65c6ccccc6-c6ccccc65)cccc4c3c2)cc1] 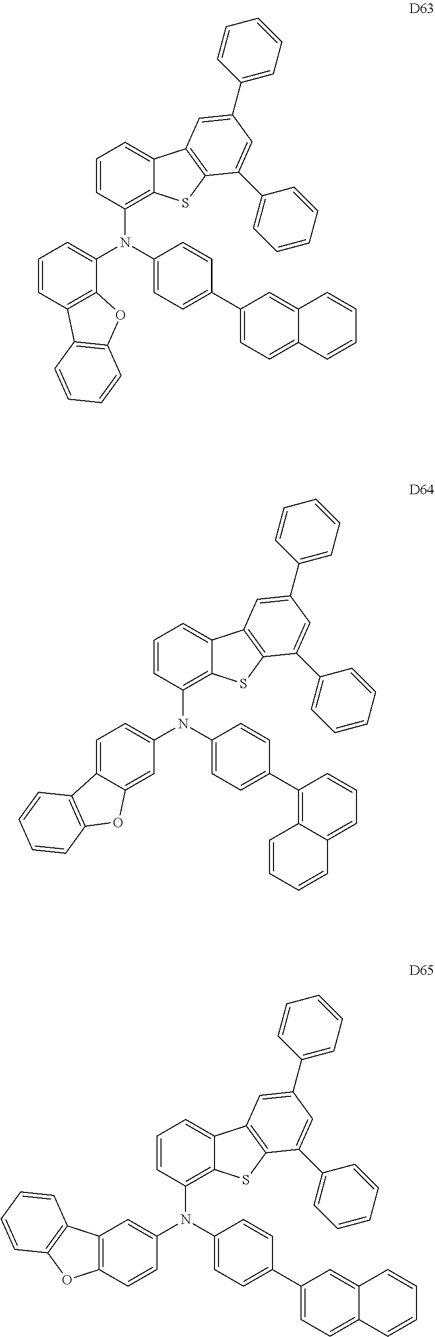 [SMILES: c1ccc(-c2cc(-c3ccccc3)c3sc4c(N(c5ccc(-c6ccc7ccccc7c6)cc5)c5cccc6c5oc5ccccc56)cccc4c3c2)cc1 ; c1ccc(-c2cc(-c3ccccc3)c3sc4c(N(c5ccc(-c6cccc7ccccc67)cc5)c5ccc6c(c5)oc5ccccc56)cccc4c3c2)cc1 ; c1ccc(-c2cc(-c3ccccc3)c3sc4c(N(c5ccc(-c6ccc7ccccc7c6)cc5)c5ccc6oc7ccccc7c6c5)cccc4c3c2)cc1] 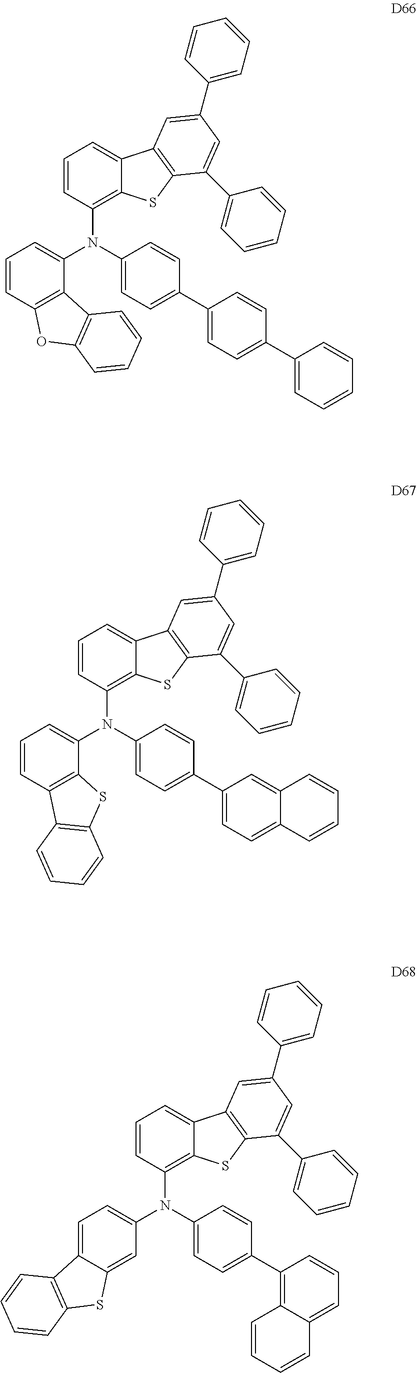 [SMILES: c1ccc(-c2ccc(-c3ccc(N(c4cccc5c4sc4c(-c6ccccc6)cc(-c6ccccc6)cc45)c4cccc5oc6ccccc6c45)cc3)cc2)cc1 ; c1ccc(-c2cc(-c3ccccc3)c3sc4c(N(c5ccc(-c6ccc7ccccc7c6)cc5)c5cccc6c5sc5ccccc56)cccc4c3c2)cc1 ; c1ccc(-c2cc(-c3ccccc3)c3sc4c(N(c5ccc(-c6cccc7ccccc67)cc5)c5ccc6c(c5)sc5ccccc56)cccc4c3c2)cc1] 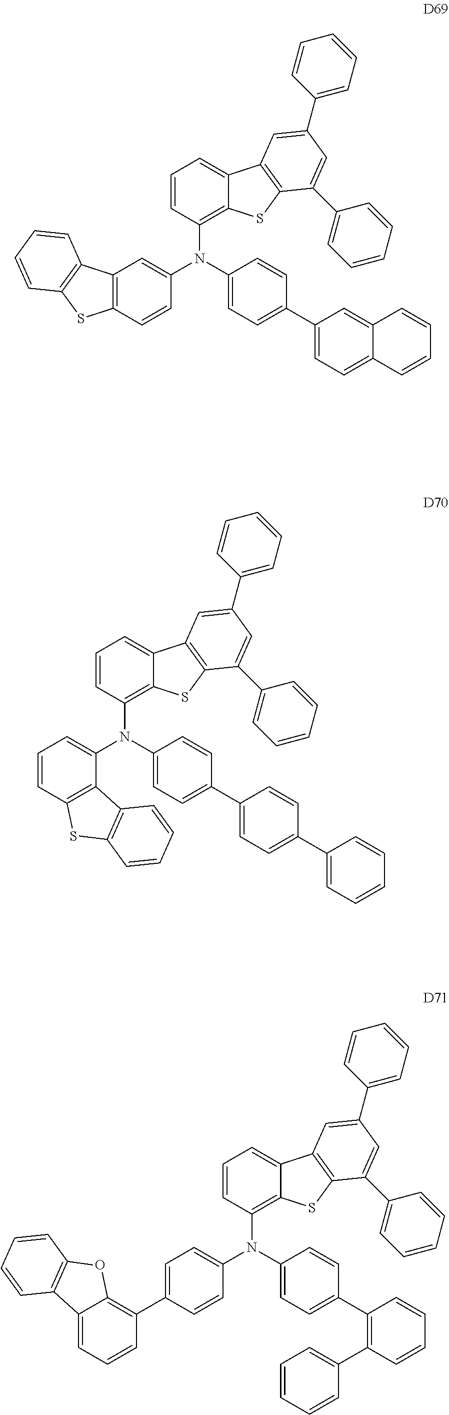 [SMILES: c1ccc(-c2cc(-c3ccccc3)c3sc4c(N(c5ccc(-c6ccc7ccccc7c6)cc5)c5ccc6sc7ccccc7c6c5)cccc4c3c2)cc1 ; c1ccc(-c2ccc(-c3ccc(N(c4cccc5c4sc4c(-c6ccccc6)cc(-c6ccccc6)cc45)c4cccc5sc6ccccc6c45)cc3)cc2)cc1 ; c1ccc(-c2cc(-c3ccccc3)c3sc4c(N(c5ccc(-c6ccccc6-c6ccccc6)cc5)c5ccc(-c6cccc7c6oc6ccccc67)cc5)cccc4c3c2)cc1] 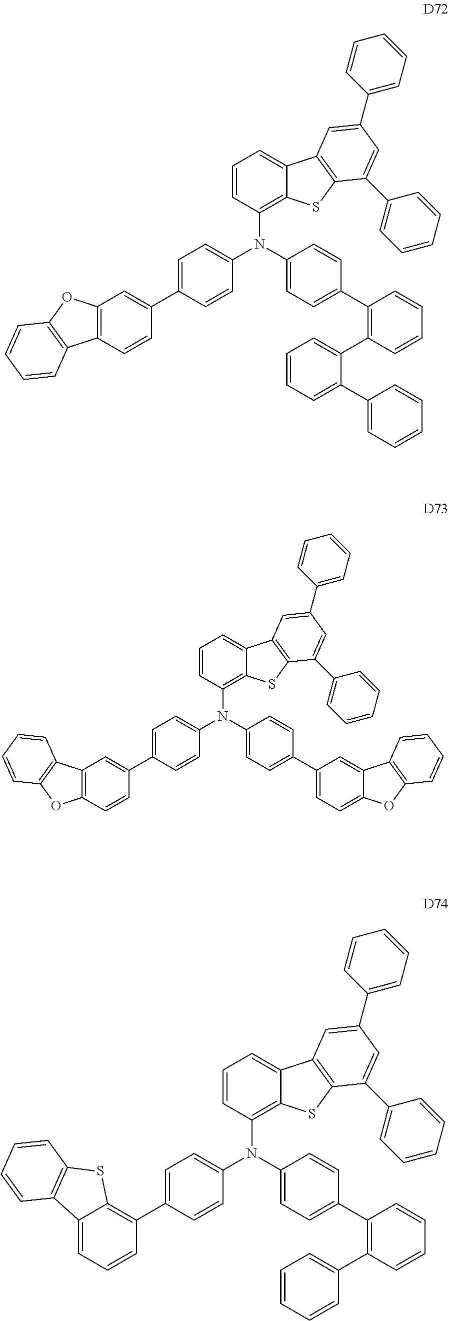 [SMILES: c1ccc(-c2cc(-c3ccccc3)c3sc4c(N(c5ccc(-c6ccc7c(c6)oc6ccccc67)cc5)c5ccc(-c6ccccc6-c6ccccc6-c6ccccc6)cc5)cccc4c3c2)cc1 ; c1ccc(-c2cc(-c3ccccc3)c3sc4c(N(c5ccc(-c6ccc7oc8ccccc8c7c6)cc5)c5ccc(-c6ccc7oc8ccccc8c7c6)cc5)cccc4c3c2)cc1 ; c1ccc(-c2cc(-c3ccccc3)c3sc4c(N(c5ccc(-c6ccccc6-c6ccccc6)cc5)c5ccc(-c6cccc7c6sc6ccccc67)cc5)cccc4c3c2)cc1] 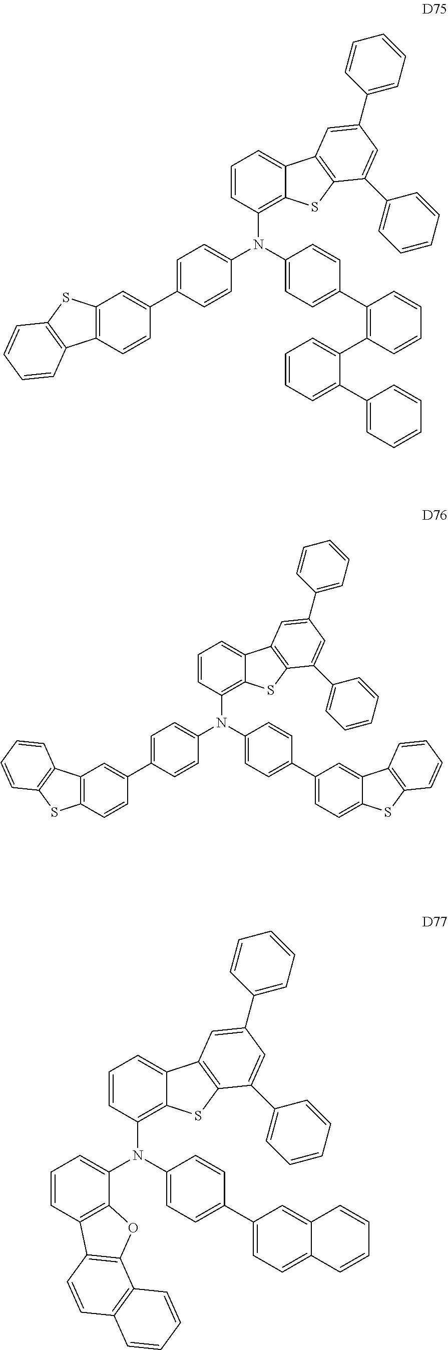 [SMILES: c1ccc(-c2cc(-c3ccccc3)c3sc4c(N(c5ccc(-c6ccc7c(c6)sc6ccccc67)cc5)c5ccc(-c6ccccc6-c6ccccc6-c6ccccc6)cc5)cccc4c3c2)cc1 ; c1ccc(-c2cc(-c3ccccc3)c3sc4c(N(c5ccc(-c6ccc7sc8ccccc8c7c6)cc5)c5ccc(-c6ccc7sc8ccccc8c7c6)cc5)cccc4c3c2)cc1 ; c1ccc(-c2cc(-c3ccccc3)c3sc4c(N(c5ccc(-c6ccc7ccccc7c6)cc5)c5cccc6c5oc5c7ccccc7ccc65)cccc4c3c2)cc1] 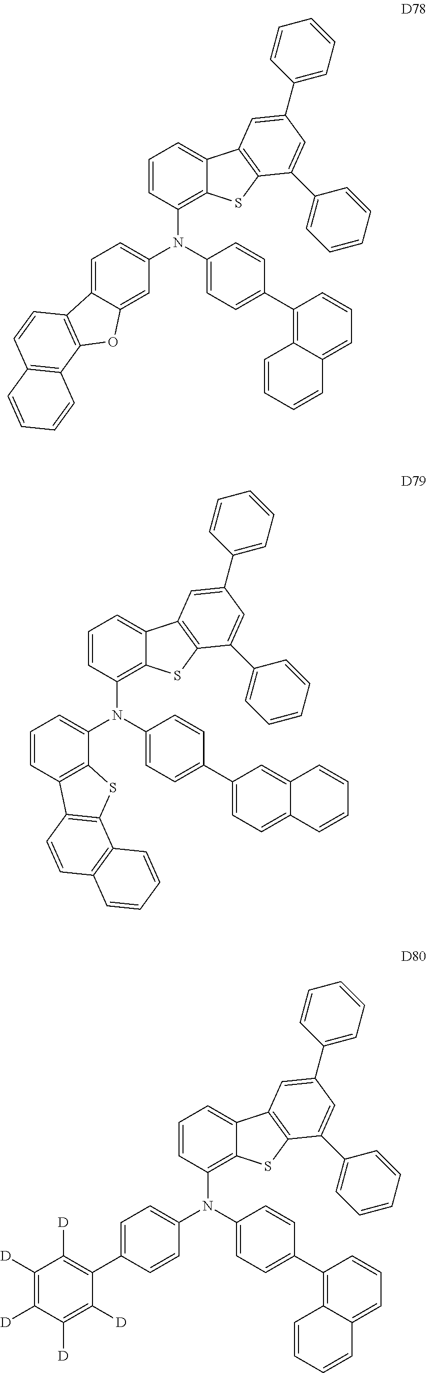 [SMILES: c1ccc(-c2cc(-c3ccccc3)c3sc4c(N(c5ccc(-c6cccc7ccccc67)cc5)c5ccc6c(c5)oc5c7ccccc7ccc65)cccc4c3c2)cc1 ; c1ccc(-c2cc(-c3ccccc3)c3sc4c(N(c5ccc(-c6ccc7ccccc7c6)cc5)c5cccc6c5sc5c7ccccc7ccc65)cccc4c3c2)cc1 ; [2H]c1c([2H])c([2H])c(-c2ccc(N(c3ccc(-c4cccc5ccccc45)cc3)c3cccc4c3sc3c(-c5ccccc5)cc(-c5ccccc5)cc34)cc2)c([2H])c1[2H]] 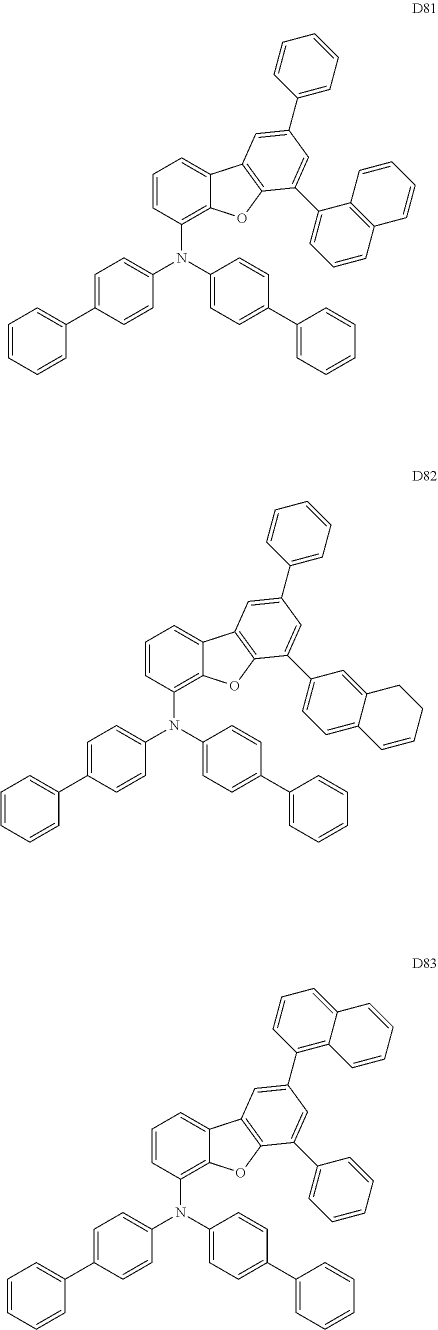 [SMILES: c1ccc(-c2ccc(N(c3ccc(-c4ccccc4)cc3)c3cccc4c3oc3c(-c5cccc6ccccc56)cc(-c5ccccc5)cc34)cc2)cc1 ; C1=Cc2ccc(-c3cc(-c4ccccc4)cc4c3oc3c(N(c5ccc(-c6ccccc6)cc5)c5ccc(-c6ccccc6)cc5)cccc34)cc2CC1 ; c1ccc(-c2ccc(N(c3ccc(-c4ccccc4)cc3)c3cccc4c3oc3c(-c5ccccc5)cc(-c5cccc6ccccc56)cc34)cc2)cc1] 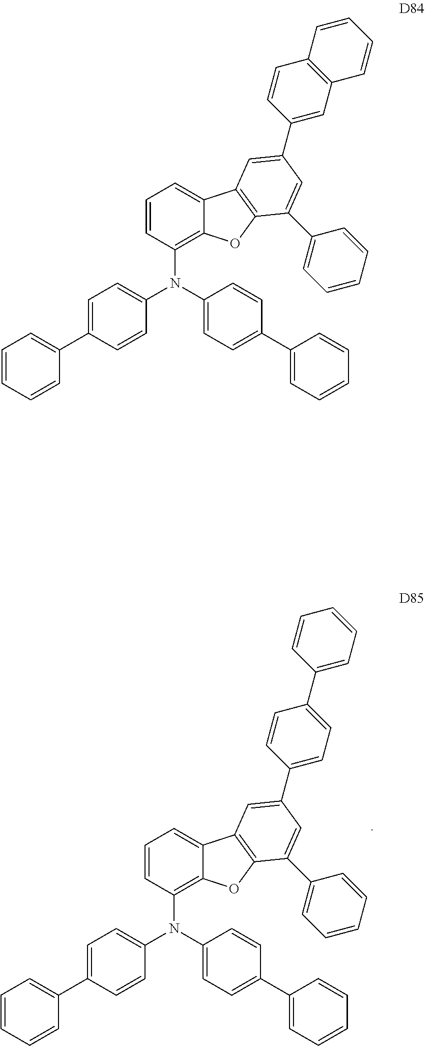 [SMILES: c1ccc(-c2ccc(N(c3ccc(-c4ccccc4)cc3)c3cccc4c3oc3c(-c5ccccc5)cc(-c5ccc6ccccc6c5)cc34)cc2)cc1 ; c1ccc(-c2ccc(-c3cc(-c4ccccc4)c4oc5c(N(c6ccc(-c7ccccc7)cc6)c6ccc(-c7ccccc7)cc6)cccc5c4c3)cc2)cc1]
